(19) United States
(12) Patent Application Publication
(10) Pub. No.: US 2026/0315487 A1
(43) Pub. Date: Oct. 15, 2026

(54) IKK-ALPHA INHIBITORS

(71) Applicant: Cancer Research Technology Limited, London (GB)

(72) Inventors: Simon P. MacKay, Glasgow (GB); Aisha A. Alsfouk, Riyadh (SA); Nahoum G. Anthony, Newcastle upon Tyne (GB); Jessica Baiget, Glasgow (GB); Giacomo Berretta, Glasgow (GB); David Breen, Glasgow (GB); Judith Huggan, Dalry (GB); Christopher Lawson, Glasgow (GB); Sabin Llona-Minguez, Glasgow (GB); Colin J. Suckling, Glasgow (GB); Christopher West, Glasgow (GB)

(21) Appl. No.: 19/481,363

(22) PCT Filed: May 3, 2024
(86) PCT No.: PCT/GB2024/051180

§ 371 Date: Nov. 4, 2025

(30) Foreign Application Priority Data
Apr. 5, 2023 (GB) 2306601.2

(51) Int. Cl. A61K 31/541; A61K 31/437; A61K 31/4375; A61K 31/4439; A61K 31/444; A61K 31/4545; A61K 31/496; A61K 31/506; A61K 31/5377; C07D 401/04; C07D 401/14; C07D 405/14; C07D 417/14; C07D 471/04; C07D 471/14

(52) U.S. Cl. CPC A61K 31/541; A61K 31/437; A61K 31/4375; A61K 31/4439; A61K 31/444; A61K 31/4545; A61K 31/496; A61K 31/506; A61K 31/5377; C07D 401/04; C07D 401/14; C07D 405/14; C07D 417/14; C07D 471/04; C07D 471/14

(57) ABSTRACT

The present invention relates to IKK-alpha inhibitory compounds, or a pharmaceutically acceptable salts, hydrates or solvates thereof, having the structural Formula (I), shown below:

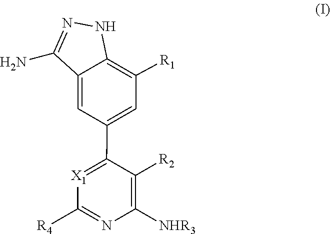

wherein R₁, R₂, R₃ R₄ and X₁ are each as defined herein. The present invention also relates to processes for the preparation of these compounds, to pharmaceutical compositions comprising them, and to their use in the treatment of proliferative disorders, such as cancer, as well as other diseases or conditions in which IKK-alpha activity is implicated.

### INTRODUCTION

The present invention relates to certain compounds that function as inhibitors of inhibitory-κB kinase (IKK) activity, and especially the alpha subunit of IKK (IKKα). The compounds of the present invention may therefore be used to treat disease or conditions mediated, at least in part, by aberrant or inappropriate IKK (and especially IKKα) activity. Cancer is an example of condition associated with aberrant or inappropriate IKK (and especially IKKα) activity. The invention furthermore relates to the use of the compounds as for treating diseases or conditions in which IKK (and especially IKKα) activity is implicated, to processes for making these compounds and to pharmaceutical compositions comprising them.

### BACKGROUND OF THE INVENTION

Cancer is caused by altered cellular proliferation. Precisely what causes a cell to become malignant and proliferate in an uncontrolled and unregulated manner has been the focus of intense research over recent decades. This research has led to the identification of molecular targets associated with key pathways that enable such malignancies.

Nuclear Factor kappa-B (NF-κB), from Nuclear factor kappa-light-chain enhancer of B-cells, represents a family of five transcription factors involved in diverse biological responses that underpin phenotypic outcomes of inflammation, modulation of immune responses, cell growth, proliferation, apoptosis and aspects of differentiation and development [1-5 ]. NF-κB signalling is now appreciated as either canonical (classical) or non-canonical (alternative) pathways via the mobilisation of both homo- and hetero-dimer complexes of these family members. Collectively the NF-κB proteins are five distinct isoforms; RelA (p65 ), ReB, c-Rel, NF-κB1 (p105 /p50 ) and NF-κB2 (p100 /p52 ) [1-5 ]. In an inactive state these proteins are typically associated with inhibitory-κB (IκB) proteins, including isoforms of IκBa, IκBβ, and IκBε and in the case of p105 and p100 proteins it is their intrinsic protein structure that maintains them in a self-bound inhibitory form by virtue of their C-terminal IκB-like structures (IκBδ and IκBγ respectively) composed of ankyrin repeats [1-5 ]. Activation and liberation of NF-κB proteins occurs typically in response to a number of extracellular ligands, as well as agents that generate a DNA Damage response (DDR), resulting in the nuclear localisation of DNA-binding protein dimers following dissociation from IκB molecules [1-5].

The canonical pathway can be activated in response to cytokines such as TNFα and IL-1 β, and pathogen-associated molecular profiles (PAMPs) such as the bacterial endotoxin lipopolysaccharide (LPS) [6, 7 ]. This response is typically rapid and transient, mediated by the classical inhibitory-κB kinase (IKK) complex (IKKα/β/γ) with a requirement for IKKβ-mediated phosphorylation of selected IκB proteins [6, 7 ]. In contrast, activation of the non-canonical NF-κB pathway is relatively slower and over a period of hours results in an IKKα-mediated liberation of predominately p52 -RelB dimers to drive gene transcription [1-7 ]. This slower response reflects reliance upon protein expression/stabilisation within the upstream components of the pathway. Whilst TNFα and IL-1 β have the ability to activate the non-canonical NF-κB pathway it is typically alternative members of the greater TNF superfamily that drive activation [3, 4 ]. This includes lymphotoxin-3 (LT-3 ), the related tumor necrosis factor superfamily member 14 (TNFSF14 ) known as LIGHT, TNF-like weak inducer of apoptosis (TWEAK), CD40 ligand (CD40 L), Receptor-activator of NF-κB ligand (RANKL) and B-cell activating factor (BAFF) [1, 3, 4].

A combination of molecular and genetic studies has shown that receptor mediated-non-canonical NF-κB activation is built around the paradigm of a TNF super family ligand activating its cognate receptor via recruitment of a sequence of identifiable adaptor molecules of the TNF-Receptor associated factor (TRAF) family, notably TRAF2 and TRAF3, modulators of ubiquitination and associated protein degradation in the form of the cellular inhibitors of apoptosis (clAPs). These proteins enable engagement and activation of the cellular kinases NF-κB-inducing kinase (NIK), the 14 ^{th} member of the MAP kinase kinase kinase (MAP3 K) family, and IKKα to determine the liberation of p52 -RelB protein complexes.

In a cellular setting, under resting non-stimulated conditions, NIK is maintained at a low expression level based upon NIK-focused proteasomal degradation. However, upon receptor activation NIK is stabilised, protein expression is increased to enable pathway activation [16 ]. It is TRAF3 that acts as the crucial regulator of NIK expression by controlling the extent of its proteasome-mediated degradation [16 ]. Upon receptor activation the focus of proteasome-mediated protein degradation switches from NIK to that of TRAF2 and TRAF3 which stabilises NIK expression to initiate the sequence of signalling events toward p100 processing [17-20 ]. The clAP proteins that function as ubiquitin ligases to ubiquitinate NIK then target TRAF3 for degradation to increase NIK protein levels.

Upon NIK protein stabilisation, as the first component of the non-canonical NF-κB pathway it catalyses is the phosphorylation of IKKα and supports IKKα recruitment to and phosphorylation of p100 to drive subsequent p100 ubiquitination and proteasome-mediated degradation to liberate p52 [16 ]. Under basal conditions p100 exists typically in dimer complexes with RelB and upon stimulated degradation generates p52 -RelB dimers able to translocate to the nucleus to initiate the transcription of distinct genes.

Both NIK and IKKα play critical roles in the phosphorylation of p100 to liberate mature p52 -RelB protein dimers. However, whilst IKKα is now viewed as the key modulator of p100 phosphorylation there is a co-dependence on NIK to deliver coupled phosphorylation and processing of p100 to generate mature p52 that is transcriptionally active [22 ]. In transfected cells, NIK can stimulate the phosphorylation, ubiquitination and processing of p100 [23, 24 ], however recombinant NIK itself does not display any phosphorylation of p100 in vitro [24, 25 ]. In the cell-based setting, NIK mediates downstream signalling by engaging and activating IKKα resulting in the phosphorylation of the C-terminal region of p100 [25 ], and this is independent of the other IKK isoforms, β and γ associated with canonical NF-κB activation [26, 27 ]. Whilst IKKα phosphorylates p100 and regulated non-canonical NF-κB activation alone, it is not as effective at inducing p100 processing as NIK [23 ]. With these observations, further studies then identified NIK to have a critical role in regulating p100 processing via the recruitment of IKKα to and binding with p100 as a protein substrate [22 ]. Collectively, NIK-IKKα interaction with p100 results in the phosphorylation of p100 at specific serine residues, primarily Ser868/870 [24 ]. These sites are components of the phospho-degron within the p100 C-terminal NIK-responsive domain (NRD) and when phosphorylated lead to βTrCP binding as part of the SCF^{βTrCP} ubiquitin ligase complex that drives the eventual processing of p100 to generate p52.

Independent of the non-canonical NF-κB pathway, a number of studies have identified that at the NIK-IKKα kinase level there are also examples of signal bifurcation. These can be dependent on differing extracellular conditions [29 ] and demonstrate that p100 is not the only substrate for IKKα-mediated phosphorylation. IKKα via catalysed phosphorylation, regulates directly a number of cellular proteins that then either directly or indirectly regulate cellular transcription [6, 7 ]. This includes transcription factors distinct from the NF-κB family, for example E2 F1 [30, 31 ], β-catenin [32 ], CBP [33 ], as well as the suppressors of transcription such as the silencing mediator for retinoic acid and thyroid hormone receptor (SMRT) [34 ] and cell cycle regulator cyclin D1 [35 ]. Additional substrates also include the Protein inhibitor of activated STAT1 (PIAS1 ) as a modulator of transcription/inflammation [36 ], the oestrogen (ER) [37 ] and androgen receptors (AR) [38 ] of the steroid hormone family receptor along with their associated steroid receptor co-factor (SRC)-3 [37, 39, 40 ] and Aurora kinase A [41, 42 ] that contributes to the mitotic process. Direct modulation of the status of these proteins by IKKα has bearing on the transcription of additional regulatory proteins such as p53 [43, 44 ] and EZH2 [44 ] and additional mitotic kinase Polo-like kinase (PLK) 4 [45 ]. IKKα therefore serves as a key switch in the coordinated regulation of both NF-κB-dependent and NF-κB-independent gene transcription and this underpins the outcomes associated with events that initiate and/or perpetuate the development of acquired characteristics, or phenotypes, we now recognise as cancer ‘Hallmarks’ as identified and defined by Hanahan & Weinberg [46, 47 ]. The transcriptional modulation driven by IKKα-mediated signalling, divulged using a number of experimental approaches such as genetic deletion and reconstitution [48, 49 ], siRNA ‘run-down’ [35 ] and over-expression strategies [50 ], may be in excess of 200 genes and these gene/protein induction/repression events support the acquisition of characteristics of specific ‘Hallmarks’, particularly the ability of tumours to ‘sustain proliferative signalling’, ‘resist cell death’, ‘evade growth suppressors’ and encourage ‘genomic instability and mutation’. More striking is the role of IKKα in regulating genes/protein that help to underpin the phenotypes associated with longer term tumour development: ‘inducing angiogenesis’ and ‘activating invasion and metastasis’ by way of regulating cytokine (e.g. IL-1 p, IL-6 [48, 49 ]) and chemokine (e.g. CCL19, CCL21, CXCL12, CXCL13 and BAFF [27, 51, 52 ]) induction and modulation of adhesion molecule (e.g. VCAM; [48-50 ]), maspin [50; 53 ] and MMPs [50 ] expression in different cellular/tissue situations. It is also evident that in particular sub-types of cancer the acquisition of a specific mutation, C250 T in the hTERT promoter [54 ] that supports tumour reactivation has identified the potential for tumours to become ‘addicted’ to IKKα-mediated non-canonical NF-κB signalling thus ‘enabling replicative potential’. Collectively, perturbation of this enzyme could have wide-ranging effects on the multiple hallmarks of tumour cells described above. Moreover, given the impact of IKKα in regulating major cytokine, chemokine and matrix metalloproteinase isoforms, intervention against this enzyme may have significant effects on tumour-stromal communication and matrix composition within the tumour microenvironment and define a better understanding of ‘tumour-promoting inflammation’.

Additional complexities to the regulation of IKKα-dependent, NF-κB-dependent and -independent gene transcription are also now apparent in the cancer setting, as we now appreciate that this transcriptional process is not wholly driven by receptor-mediated activation. For both solid tumour (e.g. pancreatic adenocarcinomas) and haematological settings (e.g. multiple myeloma) constitutive activation of IKKα-mediated signalling has been reported as a result of modulation of expression of upstream TRAF and clAP components in the pathways or mutation in these very same components that ultimately results in constitutive activation of the pathway in the absence of agonist. Furthermore, a truncated p45 form of IKKα has been identified in a number of colorectal cancers [55, 56 ], particularly those with a recognised B-Raf^{V600E} mutant background. This drives p45 IKKα-mediated nuclear signalling in a TNF superfamily member-independent manner and so brings additional mechanistic and transcriptional diversity to tumour development, which has implications for potential intervention therapeutically.

In recent years the role of the non-canonical NF-κB pathway and IKKα within it have increasingly been implicated in the development and progression of multiple solid tumours and haematological cancers. As a consequence, there is a need and a desire to identify potentially useful IKKα inhibitors

The present invention was devised with the foregoing in mind.

### REFERENCES

[1 ] Sun S C. Non-canonical NF-κB signaling pathway. Cell Res. 2011 January;21 (I):71-85.
[2 ] Razani B, Reichardt A D, Cheng G. Non-canonical NF-κB signaling activation and regulation: principles and perspectives. Immunol Rev. 2011 November; 244 (I):44-54.
[3 ] Cildir G, Low K C, Tergaonkar V. Noncanonical NF-κB Signaling in Health and Disease. Trends Mol Med. 2016 May; 22 (5 ):414-429.
[4 ] Sun S C. The non-canonical NF-κB pathway in immunity and inflammation. Nat Rev Immunol. 2017 September;17 (9 ):545-558.
[5 ] Xia L, Tan S, Zhou Y, Lin J, Wang H, Oyang L, Tian Y, Liu L, Su M, Wang H, Cao D, Liao Q. Role of the NFκB-signaling pathway in cancer. Onco Targets Ther. 2018 Apr. 11; 11:2063-2073.
[6 ] Perkins N D. Integrating cell-signalling pathways with N F-kappaB and IKK function. Nat Rev Mol Cell Biol. 2007 January;8 (I):49-62.
[7 ] Gamble C, McIntosh K, Scott R, Ho K H, Plevin R, Paul A. Inhibitory kappa B Kinases as targets for pharmacological regulation. Br J Pharmacol. 2012 February;165 (4 ):802-19.
[16 ] Liao G, Zhang M, Harhaj E W, Sun S C. Regulation of the N F-kappaB-inducing kinase by tumor necrosis factor receptor-associated factor 3 -induced degradation. J Biol Chem. 2004 Jun. 18; 279 (25 ):26243-50.
[17 ] Vallabhapurapu S, Matsuzawa A, Zhang W, Tseng P H, Keats J J, Wang H, Vignali D A, Bergsagel P L, Karin M. Nonredundant and complementary functions of TRAF2 and TRAF3 in a ubiquitination cascade that activates NIK-dependent alternative N F-kappaB signaling. Nat Immunol. 2008 December; 9 (12 ):1364-70.
[18 ] Vince J E, Wong W W, Khan N, Feltham R, Chau D, Ahmed A U, Benetatos C A, Chunduru S K, Condon S M, McKinlay M, Brink R, Leverkus M, Tergaonkar V, Schneider P, Callus B A, Koentgen F, Vaux D L, Silke J. IAP antagonists target cIAP1 to induce TNFalpha-dependent apoptosis. Cell. 2007 Nov. 16; 131 (4 ):682-93.
[19 ] Varfolomeev E, Blankenship J W, Wayson S M, Fedorova A V, Kayagaki N, Garg P, Zobel K, Dynek J N, Elliott L O, Wallweber H J, Flygare J A, Fairbrother W J, Deshayes K, Dixit V M, Vucic D. IAP antagonists induce autoubiquitination of c-IAPs, N F-kappaB activation, and TNFalpha-dependent apoptosis. Cell. 2007 Nov. 16; 131 (4 ):669-81
[20 ] Zarnegar B J, Wang Y, Mahoney D J, Dempsey P W, Cheung H H, He J, Shiba T, Yang X, Yeh W C, Mak T W, Korneluk R G, Cheng G. Noncanonical N F-kappaB activation requires coordinated assembly of a regulatory complex of the adaptors cIAP1, cIAP2, TRAF2 and TRAF3 and the kinase NIK. Nat Immunol. 2008 December; 9 (12 ):1371-8
[22 ] Xiao G, Fong A, Sun S C. Induction of p100 processing by N F-kappaB-inducing kinase involves docking IkappaB kinase alpha (IKKα lpha) to p100 and IKKα lpha-mediated phosphorylation. J Biol Chem. 2004 Jul. 16; 279 (29 ):30099-105
[23 ] Xiao G, Harhaj E W, Sun S C. N F-kappaB-inducing kinase regulates the processing of N F-kappaB2 p100 . Mol Cell. 2001 February;7 (2 ):401-9.
[24 ] Liang C, Zhang M, Sun S C. beta-TrCP binding and processing of N F-kappaB2 /p100 involve its phosphorylation at serines 866 and 870 . Cell Signal. 2006 August; 18 (8 ):1309-17
[25 ] Senftleben U, Cao Y, Xiao G, Greten F R, Krahn G, Bonizzi G, Chen Y, Hu Y, Fong A, Sun S C, Karin M. Activation by IKKα lpha of a second, evolutionary conserved, N F-kappa B signaling pathway. Science. 2001 Aug. 24; 293 (5534 ):1495-9.
[26 ] Claudio E, Brown K, Park S, Wang H, Siebenlist U. BAFF-induced NEMO-independent processing of N F-kappa B2 in maturing B cells. Nat Immunol. 2002October;3 (10 ):958-65
[27 ] Dejardin E, Droin N M, Delhase M, Haas E, Cao Y, Makris C, Li Z W, Karin M, Ware C F, Green D R. The lymphotoxin-beta receptor induces different patterns of gene expression via two N F-kappaB pathways. Immunity. 2002October;17 (4 ):525-35.
[29 ] Wang R P, Zhang M, Li Y, Diao F C, Chen D, Zhai Z, Shu H B. Differential regulation of IKK alpha-mediated activation of IRF3/7 by NIK. Mol Immunol. 2008 April; 45 (7 ):1926-34.
[30 ] Tu Z, Prajapati S, Park K J, Kelly N J, Yamamoto Y, Gaynor R B. IKK alpha regulates estrogen-induced cell cycle progression by modulating E2 F1 expression. J Biol Chem. 2006 Mar. 10; 281 (10 ):6699-706.
[31 ] Ammirante M, Kuraishy A I, Shalapour S, Strasner A, Ramirez-Sanchez C, Zhang W, Shabaik A, Karin M. An IKKα-E2 F1 -BM11 cascade activated by infiltrating B cells controls prostate regeneration and tumor recurrence. Genes Dev. 2013 Jul. 1; 27 (13 ):1435-40.
[32 ] Lamberti C, Lin K M, Yamamoto Y, Verma U, Verma I M, Byers S, Gaynor R B. Regulation of beta-catenin function by the IkappaB kinases. J Biol Chem. 2001 Nov. 9; 276 (45 ):42276-86
[33 ] Huang W C, Ju T K, Hung M C, Chen C C. Phosphorylation of CBP by IKKα lpha promotes cell growth by switching the binding preference of CBP from p53 to N F-kappaB. Mol Cell. 2007 Apr. 13; 26 (I):75-87
[34 ] Hoberg J E, Popko A E, Ramsey C S, Mayo M W. IkappaB kinase alpha-mediated derepression of SMRT potentiates acetylation of RelAlp65 by p300 . Mol Cell Biol. 2006 January;26 (2 ):457-71.
[35 ] Kwak Y T, Li R, Becerra C R, Tripathy D, Frenkel E P, Verma U N. IkappaB kinase alpha regulates subcellular distribution and turnover of cyclin D1 by phosphorylation. J Biol Chem. 2005Oct. 7; 280 (40 ):33945-52.
[36 ] Liu B, Yang Y, Chernishof V, Loo R R, Jang H, Tahk S, Yang R, Mink S, Shultz D, Bellone C J, Loo J A, Shuai K. Proinflammatory stimuli induce IKKα lpha-mediated phosphorylation of PIAS1 to restrict inflammation and immunity. Cell. 2007 Jun. 1; 129 (5 ):903-14
[37 ] Park K J, Krishnan V, O'Malley B W, Yamamoto Y, Gaynor R B. Formation of an IKKα lpha-dependent transcription complex is required for estrogen receptor-mediated gene activation. Mol Cell. 2005 Apr. 1; 18 (I):71-82
[38 ] Jain G, Voogdt C, Tobias A, Spindler K D, Möller P, Cronauer M V, Marienfeld R B. IκB kinases modulate the activity of the androgen receptor in prostate carcinoma cell lines. Neoplasia. 2012 March;14 (3 ):178-89.
[39 ] Wu R C, Qin J, Hashimoto Y, Wong J, Xu J, Tsai S Y, Tsai M J, O'Malley B W. Regulation of SRC-3 (pCIP/ACTR/AIB-1 /RAC-3 /TRAM-1 ) Coactivator activity by I kappa B kinase. Mol Cell Biol. 2002 May; 22 (10 ):3549-61.
[40 ] Wu R C, Qin J, Yi P, Wong J, Tsai S Y, Tsai M J, O'Malley B W. Selective phosphorylations of the SRC-3 /AIB1 coactivator integrate genomic reponses to multiple cellular signaling pathways. Mol Cell. 2004 Sep. 24; 15 (6 ):937-49.
[41 ] Prajapati S, Tu Z, Yamamoto Y, Gaynor R B. IKKα lpha regulates the mitotic phase of the cell cycle by modulating Aurora A phosphorylation. Cell Cycle. 2006October;5 (20 ):2371-80.
[42 ] Irelan J T, Murphy T J, DeJesus P D, Teo H, Xu D, Gomez-Ferreria M A, Zhou Y, Miraglia L J, Rines D R, Verma I M, Sharp D J, Tergaonkar V, Chanda S K. A role for IkappaB kinase 2 in bipolar spindle assembly. Proc Natl Acad Sci USA. 2007Oct. 23; 104 (43 ):16940-5.
[43 ] Schumm K, Rocha S, Caamano J, Perkins N D. Regulation of p53 tumour suppressor target gene expression by the p52 N F-kappaB subunit. EMBO J. 2006Oct. 18; 25 (20 ):4820-32.
[44 ] lannetti A, Ledoux A C, Tudhope S J, Sellier H, Zhao B, Mowla S, Moore A, Hummerich H, Gewurz B E, Cockell S J, Jat P S, Willmore E, Perkins N D. Regulation of p53 and Rb links the alternative NF-κB pathway to EZH2 expression and cell senescence. PLoS Genet. 2014 Sep. 25; 10 (9 ):e1004642
[45 ] Ledoux A C, Sellier H, Gillies K, lannetti A, James J, Perkins N D. NFκB regulates expression of Polo-like kinase 4 . Cell Cycle. 2013 Sep. 15; 12 (18 ):3052-62
[46 ] Hanahan D, Weinberg R A. The hallmarks of cancer. Cell. 2000 Jan. 7; 100 (I):57-70.
[47 ] Hanahan D, Weinberg R A. Hallmarks of cancer: the next generation. Cell. 2011 Mar. 4; 144 (5 ):646-74.
[48 ] Li X, Massa P E, Hanidu A, Peet G W, Aro P, Savitt A, Mische S, Li J, Marcu K B. IKKα lpha, IKKbeta, and NEMO/lKKgamma are each required for the N F-kappa B-mediated inflammatory response program. J Biol Chem. 2002 Nov. 22; 277 (47 ):45129-40.
[49 ] Massa P E, Li X, Hanidu A, Siamas J, Pariali M, Pareja J, Savitt A G, Catron K M, Li J, Marcu K B. Gene expression profiling in conjunction with physiological rescues of IKKα lpha-null cells with wild type or mutant IKKα lpha reveals distinct classes of IKKα lpha/N F-kappaB-dependent genes. J Biol Chem. 2005 Apr. 8; 280 (14 ):14057-69
[50 ] Nadiminty N, Dutt S, Tepper C, Gao A C. Microarray analysis reveals potential target genes of N F-kappaB2 /p52 in LNCaP prostate cancer cells. Prostate. 2010 Feb. 15; 70 (3 ):276-87.
[51 ] Wharry C E, Haines K M, Carroll R G, May M J. Constitutive non-canonical NFkappaB signaling in pancreatic cancer cells. Cancer Biol Ther. 2009 August; 8 (16 ):1567-76.
[52 ] Ammirante M, Shalapour S, Kang Y, Jamieson C A, Karin M. Tissue injury and hypoxia promote malignant progression of prostate cancer by inducing CXCL13 expression in tumor myofibroblasts. Proc Natl Acad Sci USA. 2014Oct. 14; 111 (41 ):14776-81.
[53 ] Ammirante M, Luo J L, Grivennikov S, Nedospasov S, Karin M. B-cell-derived lymphotoxin promotes castration-resistant prostate cancer. Nature. 2010 Mar. 11; 464 (7286 ):302-5.
[54 ] Li Y, Zhou O L, Sun W, Chandrasekharan P, Cheng H S, Ying Z, Lakshmanan M, Raju A, Tenen D G, Cheng S Y, Chuang K H, Li J, Prabhakar S, Li M, Tergaonkar V. Non-canonical N F-κB signalling and ETS1/2 cooperatively drive C250 T mutant TERT promoter activation. Nat Cell Biol. 2015October;17 (10 ):1327-38.
[55 ] Margalef P, Fernández-Majada V, Villanueva A, Garcia-Carbonell R, Iglesias M, Lòpez L, Martinez-Iniesta M, Villà-Freixa J, Mulero M C, Andreu M, Torres F, Mayo M W, Bigas A, Espinosa L. A truncated form of IKKα is responsible for specific nuclear IKK activity in colorectal cancer. Cell Rep. 2012Oct. 25; 2 (4 ):840-54.
[56 ] Margalef P, Colomer C, Villanueva A, Montagut C, Iglesias M, Bellosillo B, Salazar R, Martínez-Iniesta M, Bigas A, Espinosa L. BRAF-induced tumorigenesis is IKKα-dependent but NF-κB-independent. Sci Signal. 2015 Apr. 21; 8 (373 ):ra38.

### SUMMARY OF THE INVENTION

According to a first aspect of the present invention there is provided a compound, or a pharmaceutically acceptable salt, hydrate or solvate thereof, as defined herein.

According to a further aspect of the present invention, there is provided a pharmaceutical composition comprising a compound as defined herein, or a pharmaceutically acceptable salt, hydrate or solvate thereof, in admixture with a pharmaceutically acceptable diluent or carrier.

According to a further aspect of the present invention, there is provided a method of inhibiting IKKα activity, in vitro or in vivo, said method comprising contacting a cell with an effective amount of a compound or a pharmaceutically acceptable salt, hydrate or solvate thereof as defined herein.

According to a further aspect of the present invention, there is provided a method of treating a disease or disorder in which IKKα activity is implicated in a patient in need of such treatment, said method comprising administering to said patient a therapeutically effective amount of a compound or a pharmaceutically acceptable salt, hydrate or solvate thereof as defined herein, or a pharmaceutical composition as defined herein.

According to a further aspect of the present invention, there is provided a method of treating a proliferative disorder in a patient in need of such treatment, said method comprising administering to said patient a therapeutically effective amount of a compound or a pharmaceutically acceptable salt, hydrate or solvate thereof as defined herein, or a pharmaceutical composition as defined herein.

According to a further aspect of the present invention, there is provided a method of treating cancer in a patient in need of such treatment, said method comprising administering to said patient a therapeutically effective amount of a compound or a pharmaceutically acceptable salt, hydrate or solvate thereof as defined herein, or a pharmaceutical composition as defined herein.

According to a further aspect of the present invention, there is provided a compound, or a pharmaceutically acceptable salt, hydrate or solvate thereof, or a pharmaceutical composition as defined herein, for use in therapy.

According to a further aspect of the present invention, there is provided a compound, or a pharmaceutically acceptable salt, hydrate or solvate thereof, or a pharmaceutical composition as defined herein, for use as a medicament.

According to a further aspect of the present invention, there is provided a compound or a pharmaceutically acceptable salt, hydrate or solvate thereof as defined herein, or a pharmaceutical composition as defined herein, for use in the treatment of a proliferative disorder.

According to a further aspect of the present invention, there is provided a compound, or a pharmaceutically acceptable salt, hydrate or solvate thereof, or a pharmaceutical composition as defined herein for use in the treatment of cancer. In a particular embodiment, the cancer is human cancer.

According to a further aspect of the present invention, there is provided a compound, or a pharmaceutically acceptable salt, hydrate or solvate thereof, as defined herein for use in the inhibition of IKKα activity.

According to a further aspect of the present invention, there is provided a compound, or a pharmaceutically acceptable salt, hydrate or solvate thereof, as defined herein for use in the treatment of a disease or disorder in which IKKα activity is implicated.

According to a further aspect of the present invention, there is provided the use of a compound, or a pharmaceutically acceptable salt, hydrate or solvate thereof, as defined herein in the manufacture of a medicament for the treatment of a proliferative disorder.

According to a further aspect of the present invention, there is provided the use of a compound, or a pharmaceutically acceptable salt, hydrate or solvate thereof, as defined herein in the manufacture of a medicament for the treatment of cancer.

According to a further aspect of the present invention, there is provided a use of a compound, or a pharmaceutically acceptable salt, hydrate or solvate thereof, as defined herein in the manufacture of a medicament for the inhibition of IKKα activity.

According to a further aspect of the present invention, there is provided a use of a compound, or a pharmaceutically acceptable salt, hydrate or solvate thereof, as defined herein in the manufacture of a medicament for the treatment of a disease or disorder in which IKKα activity is implicated.

According to a further aspect of the present invention, there is provided a process for preparing a compound, or a pharmaceutically acceptable salt, hydrate or solvate thereof, as defined herein.

According to a further aspect of the present invention, there is provided a compound, or a pharmaceutically acceptable salt, hydrate or solvate thereof, obtainable by, or obtained by, or directly obtained by a process of preparing a compound as defined herein.

According to a further aspect of the present invention, there are provided novel intermediates as defined herein which are suitable for use in any one of the synthetic methods set out herein.

Features, including optional, suitable, and preferred features in relation to one aspect of the invention may also be features, including optional, suitable and preferred features in relation to any other aspect of the invention.

### DETAILED DESCRIPTION OF THE INVENTION

### Definitions

Unless otherwise stated, the following terms used in the specification and claims have the following meanings set out below.

It is to be appreciated that references to “treating” or “treatment” include prophylaxis as well as the alleviation of established symptoms of a condition. “Treating” or “treatment” of a state, disorder or condition therefore includes: (I) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2 ) inhibiting the state, disorder or condition, i.e., arresting, reducing or delaying the development of the disease or a relapse thereof (in case of maintenance treatment) or at least one clinical or subclinical symptom thereof, or (3 ) relieving or attenuating the disease, i.e., causing regression of the state, disorder or condition or at least one of its clinical or subclinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated. It should be understood that in, for example, a human or other mammal, a therapeutically effective amount can be determined experimentally in a laboratory or clinical setting, or a therapeutically effective amount may be the amount required by the guidelines of the United States Food and Drug Administration (FDA) or equivalent foreign regulatory body, for the particular disease and subject being treated. It should be appreciated that determination of proper dosage forms, dosage amounts, and routes of administration is within the level of ordinary skill in the pharmaceutical and medical arts.

As used herein by themselves or in conjunction with another term or terms, “subject(s)” and “patient(s)”, refer to animals (e.g. mammals), particularly humans. Suitably, the “subject(s)” and “patient(s)” may be a non-human animal (e.g. livestock and domestic pets) or a human.

As used herein by itself or in conjunction with another term or terms, “pharmaceutically acceptable” refers to materials that are generally chemically and/or physically compatible with other ingredients (such as, for example, with reference to a formulation), and/or is generally physiologically compatible with the recipient (such as, for example, a subject) thereof.

In this specification the term “alkyl” includes both straight and branched chain alkyl groups. References to individual alkyl groups such as “propyl” are specific for the straight chain version only and references to individual branched chain alkyl groups such as “isopropyl” are specific for the branched chain version only. For example, “(1-6 C)alkyl” includes (1-4 C)alkyl, (1 -3 C)alkyl, propyl, isopropyl and t-butyl.

The term “(m-nC)” or “(m-nC) group” used alone or as a prefix, refers to any group having m to n carbon atoms.

An “alkylene” group is an alkyl group that is positioned between and serves to connect two other chemical groups. Thus, “(1-6 C)alkylene” means a linear saturated divalent hydrocarbon radical of one to six carbon atoms or a branched saturated divalent hydrocarbon radical of three to six carbon atoms, for example, methylene (—CH₂—), the ethylene isomers (—CH(CH₃ )- and —CH₂ CH₂—), the propylene isomers (—CH(CH₃ )CH₂—, —CH(CH₂ CH₃ )—, —C(CH₃ )₂—, and —CH₂ CH₂ CH₂—), pentylene (—CH₂ CH₂ CH₂ CH₂ CH₂—), and the like.

The term “alkyenyl” refers to straight and branched chain alkyl groups comprising 2 or more carbon atoms, wherein at least one carbon-carbon double bond is present within the group. Examples of alkenyl groups include ethenyl, propenyl and but-2,3 -enyl and includes all possible geometric (E/Z) isomers.

The term “alkynyl” refers to straight and branched chain alkyl groups comprising 2 or more carbon atoms, wherein at least one carbon-carbon triple bond is present within the group. Examples of alkynyl groups include acetylenyl and propynyl.

“(m-nC)cycloalkyl” means a saturated hydrocarbon ring system containing from m to n number of carbon atoms. Exemplary cycloalkyl groups include, for example, cyclopropyl, cyclobutyl, cyclopentyl, cyclohexyl, cycloheptyl and bicyclo[2.2.1 ]heptyl.

The term “alkoxy” refers to O-linked straight and branched chain alkyl groups. Examples of alkoxy groups include methoxy, ethoxy and t-butoxy.

The term “haloalkyl” is used herein to refer to an alkyl group in which one or more hydrogen atoms have been replaced by halogen (e.g. fluorine) atoms. Examples of haloalkyl groups include —CH₂ F, —CHF₂ and —CF₃ .

The term “halo” or “halogeno” refers to fluoro, chloro, bromo and iodo, suitably fluoro, chloro and bromo, more suitably, fluoro and chloro.

The term “carbocyclyl”, “carbocyclic” or “carbocycle” means a non-aromatic saturated or partially saturated monocyclic, fused, bridged, or spiro bicyclic carbon-containing ring system(s). Monocyclic carbocyclic rings contain from about 3 to 12 (suitably from 3 to 7 ) ring atoms. Bicyclic carbocycles contain from 6 to 17 member atoms, suitably 7 to 12 member atoms, in the ring. Bicyclic carbocyclic(s) rings may be fused, spiro, or bridged ring systems. Examples of carbocyclic groups include cyclopropyl, cyclobutyl, cyclohexyl, cyclohexenyl and spiro[3.3 ]heptanyl.

The term “heterocyclyl”, “heterocyclic” or “heterocycle” means a non-aromatic saturated or partially saturated monocyclic, fused, bridged, or spiro bicyclic heterocyclic ring system(s). Monocyclic heterocyclic rings contain from about 3 to 12 (suitably from 3 to 7 ) ring atoms, with from 1 to 5 (suitably 1, 2 or 3 ) heteroatoms selected from nitrogen, oxygen or sulfur in the ring. Bicyclic heterocycles contain from 7 to 17 member atoms, suitably 7 to 12 member atoms, in the ring. Bicyclic heterocyclic(s) rings may be fused, spiro, or bridged ring systems. Examples of heterocyclic groups include cyclic ethers such as oxiranyl, oxetanyl, tetrahydrofuranyl, dioxanyl, and substituted cyclic ethers. Heterocycles containing nitrogen include, for example, azetidinyl, pyrrolidinyl, piperidinyl, piperazinyl, tetrahydrotriazinyl, tetrahydropyrazolyl, and the like. Typical sulfur containing heterocycles include tetrahydrothienyl, dihydro-1,3 -dithiol, tetrahydro-2 H-thiopyran, and hexahydrothiepine. Other heterocycles include dihydro-oxathiolyl, tetrahydro-oxazolyl, tetrahydro-oxadiazolyl, tetrahydrodioxazolyl, tetrahydro-oxathiazolyl, hexahydrotriazinyl, tetrahydro-oxazinyl, morpholinyl, thiomorpholinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothienyl and thiomorpholinyl such as tetrahydrothiene 1,1 -dioxide and thiomorpholinyl 1,1 -dioxide.

Heterocycles may comprise 1 or 2 oxo (═O) or thioxo (═S) substituents. A suitable value for a heterocyclyl group which bears 1 or 2 oxo (═O) or thioxo (═S) substituents is, for example, 2 -oxopyrrolidinyl, 2 -thioxopyrrolidinyl, 2 -oxoimidazolidinyl, 2 -thioxoimidazolidinyl, 2 -oxopiperidinyl, 2,5 -dioxopyrrolidinyl, 2,5 -dioxoimidazolidinyl or 2,6 -dioxopiperidinyl. Particular heterocyclyl groups are saturated monocyclic 3 to 7 membered heterocyclyls containing 1, 2 or 3 heteroatoms selected from nitrogen, oxygen or sulfur, for example azetidinyl, tetrahydrofuranyl, tetrahydropyranyl, pyrrolidinyl, morpholinyl, tetrahydrothienyl, tetrahydrothienyl 1,1 -dioxide, thiomorpholinyl, thiomorpholinyl 1,1 -dioxide, piperidinyl, homopiperidinyl, piperazinyl or homopiperazinyl. As the skilled person would appreciate, any heterocycle may be linked to another group via any suitable atom, such as via a carbon or nitrogen atom. However, reference herein to piperidino or morpholino refers to a piperidin-1 -yl or morpholin-4 -yl ring that is linked via the ring nitrogen.

By “bridged ring systems” is meant ring systems in which two rings share more than two atoms, see for example Advanced Organic Chemistry, by Jerry March, 4^{th} Edition, Wiley Interscience, pages 131-133, 1992 . Examples of bridged heterocyclyl ring systems include, aza-bicyclo[2.2.1 ]heptane, 2 -oxa-5 -azabicyclo[2.2.1 ]heptane, aza-bicyclo[2.2.2 ]octane, aza-bicyclo[3.2.1 ]octane and quinuclidine.

By “spiro bi-cyclic ring systems” we mean that the two ring systems share one common spiro carbon atom, i.e. the heterocyclic ring is linked to a further carbocyclic or heterocyclic ring through a single common spiro carbon atom. Examples of spiro ring systems include 6 -azaspiro[3.4 ]octane, 2 -oxa-6 -azaspiro[3.4 ]octane, 2 -azaspiro[3.3 ]heptanes, 2 -oxa-6 -azaspiro[3.3 ]heptanes, 7 -oxa-2 -azaspiro[3.5 ]nonane, 6 -oxa-2 -azaspiro[3.4 ]octane, 2 -oxa-7 -azaspiro[3.5 ]nonane and 2 -oxa-6 -azaspiro[3.5 ]nonane.

As used herein by itself or in conjunction with another term or terms, “aromatic” refers to monocyclic and polycyclic ring systems containing 4 n+2 pi electrons, where n is an integer. Aromatic should be understood as referring to and including ring systems that contain only carbon atoms (i.e. “aryl”) as well as ring systems that contain at least one heteroatom selected from N, O or S (i.e. “heteroaromatic” or “heteroaryl”). An aromatic ring system can be substituted or unsubstituted.

As used herein by itself or in conjunction with another term or terms, “non-aromatic” refers to a monocyclic or polycyclic ring system having at least one double bond that is not part of an extended conjugated pi system. As used herein, non-aromatic refers to and includes ring systems that contain only carbon atoms as well as ring systems that contain at least one heteroatom selected from N, O or S. A non-aromatic ring system can be substituted or unsubstituted.

The term “heteroaryl” or “heteroaromatic” means an aromatic mono-, bi-, or polycyclic ring incorporating one or more (for example 1-4, particularly 1, 2 or 3 ) heteroatoms selected from nitrogen, oxygen or sulfur. The term heteroaryl includes both monovalent species and divalent species. Examples of heteroaryl groups are monocyclic and bicyclic groups containing from five to twelve ring members, and more usually from five to ten ring members. The heteroaryl group can be, for example, a 5 - or 6 -membered monocyclic ring or a 9 - or 10 -membered bicyclic ring, for example a bicyclic structure formed from fused five and six membered rings or two fused six membered rings. Each ring may contain up to about four heteroatoms typically selected from nitrogen, sulfur and oxygen. Typically the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five.

Examples of heteroaryl include furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5 -triazenyl, benzofuranyl, indolyl, isoindolyl, benzothienyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl, pteridinyl, naphthyridinyl, carbazolyl, phenazinyl, benzisoquinolinyl, pyridopyrazinyl, thieno[2,3 -b]furanyl, 2 H-furo[3,2 -b]-pyranyl, 5 H-pyrido[2,3 -b]-o-oxazinyl, 1 H-pyrazolo[4,3 -d]-oxazolyl, 4 H-imidazo[4,5 -d]thiazolyl, pyrazino[2,3 -b]pyridazinyl, imidazo[2,1 -b]thiazolyl, imidazo[1,2 -b][1,2,4 ]triazinyl. “Heteroaryl” also covers partially aromatic bi- or polycyclic ring systems wherein at least one ring is an aromatic ring and one or more of the other ring(s) is a non-aromatic, saturated or partially saturated ring, provided at least one ring contains one or more heteroatoms selected from nitrogen, oxygen or sulfur. Examples of partially aromatic heteroaryl groups include for example, tetrahydroisoquinolinyl, tetrahydroquinolinyl, 2 -oxo-1,2,3,4 -tetrahydroquinolinyl, dihydrobenzthienyl, dihydrobenzfuranyl, 2,3 -dihydro-benzo[1,4 ]dioxinyl, benzo[1,3 ]dioxolyl, 2,2 -dioxo-1,3 -dihydro-2 -benzothienyl, 4,5,6,7 -tetrahydrobenzofuranyl, indolinyl, 1,2,3,4 -tetrahydro-1,8 -naphthyridinyl, 1,2,3,4 -tetrahydropyrido[2,3 -b]pyrazinyl and 3,4 -dihydro-2 H-pyrido[3,2 -b][1,4 ]oxazinyl.

Examples of five membered heteroaryl groups include but are not limited to pyrrolyl, furanyl, thienyl, imidazolyl, furazanyl, oxazolyl, oxadiazolyl, oxatriazolyl, isoxazolyl, thiazolyl, isothiazolyl, pyrazolyl, triazolyl and tetrazolyl groups.

Examples of six membered heteroaryl groups include but are not limited to pyridyl, pyrazinyl, pyridazinyl, pyrimidinyl and triazinyl.

A bicyclic heteroaryl group may be, for example, a group selected from:
a benzene ring fused to a 5 - or 6 -membered ring containing 1, 2 or 3 ring heteroatoms;
a pyridine ring fused to a 5 - or 6 -membered ring containing 1, 2 or 3 ring heteroatoms;
a pyrimidine ring fused to a 5 - or 6 -membered ring containing 1 or 2 ring heteroatoms;
a pyrrole ring fused to a 5 - or 6 -membered ring containing 1, 2 or 3 ring heteroatoms;
a pyrazole ring fused to a 5 - or 6 -membered ring containing 1 or 2 ring heteroatoms;
a pyrazine ring fused to a 5 - or 6 -membered ring containing 1 or 2 ring heteroatoms;
an imidazole ring fused to a 5 - or 6 -membered ring containing 1 or 2 ring heteroatoms;
an oxazole ring fused to a 5 - or 6 -membered ring containing 1 or 2 ring heteroatoms;
an isoxazole ring fused to a 5 - or 6 -membered ring containing 1 or 2 ring heteroatoms;
a thiazole ring fused to a 5 - or 6 -membered ring containing 1 or 2 ring heteroatoms;
an isothiazole ring fused to a 5 - or 6 -membered ring containing 1 or 2 ring heteroatoms;
a thiophene ring fused to a 5 - or 6 -membered ring containing 1, 2 or 3 ring heteroatoms;
a furan ring fused to a 5 - or 6 -membered ring containing 1, 2 or 3 ring heteroatoms;
a cyclohexyl ring fused to a 5 - or 6 -membered heteroaromatic ring containing 1, 2 or 3 ring heteroatoms; and
a cyclopentyl ring fused to a 5 - or 6 -membered heteroaromatic ring containing 1, 2 or 3 ring heteroatoms.

Particular examples of bicyclic heteroaryl groups containing a six membered ring fused to a five membered ring include but are not limited to benzfuranyl, benzthiophenyl, benzimidazolyl, benzoxazolyl, benzisoxazolyl, benzthiazolyl, benzisothiazolyl, isobenzofuranyl, indolyl, isoindolyl, indolizinyl, indolinyl, isoindolinyl, purinyl (e.g., adeninyl, guaninyl), indazolyl, benzodioxolyl and pyrazolopyridinyl groups.

Particular examples of bicyclic heteroaryl groups containing two fused six membered rings include but are not limited to quinolinyl, isoquinolinyl, chromanyl, thiochromanyl, chromenyl, isochromenyl, chromanyl, isochromanyl, benzodioxanyl, quinolizinyl, benzoxazinyl, benzodiazinyl, pyridopyridinyl, quinoxalinyl, quinazolinyl, cinnolinyl, phthalazinyl, naphthyridinyl and pteridinyl groups.

The term “aryl” means a cyclic or polycyclic aromatic ring having from 5 to 12 carbon atoms. The term aryl includes both monovalent species and divalent species. Examples of aryl groups include, but are not limited to, phenyl, biphenyl, naphthyl and the like. In a particular embodiment, an aryl is phenyl.

This specification also makes use of several composite terms to describe groups comprising more than one functionality. Such terms will be understood by a person skilled in the art. For example (3-6 C)cycloalkyl(m-nC)alkyl comprises (m-nC)alkyl substituted by (3 -6 C)cycloalkyl.

The term “optionally substituted” refers to either groups, structures, or molecules that are substituted and those that are not substituted. The term “wherein a/any CH, CH₂, CH₃ group or heteroatom (i.e. NH) within a R¹ group is optionally substituted” suitably means that (any) one of the hydrogen radicals of the R¹ group is substituted by a relevant stipulated group.

Where optional substituents are chosen from “one or more” groups it is to be understood that this definition includes all substituents being chosen from one of the specified groups or the substituents being chosen from two or more of the specified groups. In some embodiments, one or more refers to one, two or three. In another embodiment, one or more refers to one or two. In a particular embodiment, one or more refers to one.

The phrase “compound of the invention” means those compounds which are disclosed herein, both generically and specifically.

“About” when used herein in conjunction with a measurable value such as, for example, an amount or a period of time and the like, is meant to encompass reasonable variations of the value, for instance, to allow for experimental error in the measurement of said value.

### Compounds

In one aspect, the present invention relates to compounds, or pharmaceutically acceptable salts, hydrates or solvates thereof, having the structural Formula (I), shown below:

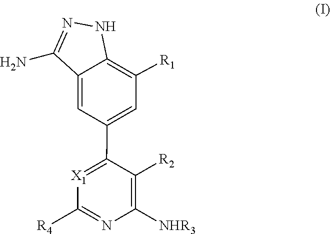

wherein:
R₁ is selected from hydrogen, halogen, (1-6 C)alkyl, (2-6 C)alkenyl, (2-6 C)alkynyl, (3 -7 C)cycloalkyl, aryl, heteroaryl and heterocyclyl,
   wherein said (1-6 C)alkyl, (2-6 C)alkenyl, (2-6 C)alkynyl, (3-7 C)cycloalkyl, aryl, heteroaryl and heterocyclyl are optionally substituted by one or more R₁₀₀ substituents;
   wherein each R₁₀₀ is independently selected from halo, (1-2 C)haloalkyl (e.g. trifluoromethyl), (1-2 C)haloalkoxy e.g. (trifluoromethoxy), cyano, hydroxyl, (1-4 C)alkyl, (1 -4 C)hydroxyalkyl, (CH₂ )ₓOR_{f}, (CH₂ )ₓ C(O)R_{f}, (CH₂ )ₓ C(O)OR_{f}, (CH₂ )ₓOC(O)R_{f}, (CH₂ )ₓ C(O)N(Rⱼ )Rₕ, (CH₂ )ₓ N(R_{g} )C(O)R_{f}, (CH₂ )ₓ S(O)_{y1} R_{f}, (CH₂ )ₓ SO₂ N(R;)Rₕ, (CH₂ )ₓ N(R_{g} )SO₂ R_{f}, (CH₂ )ₓ NRⱼ Rₕ, (CH₂ )ₓ (3-7 C)cycloalkyl, (CH₂ )ₓ heterocyclyl, (CH₂ )ₓ heteroaryl, (CH₂ )ₓ aryl;
   and wherein:
   (i) R_{f} and R_{g} are each independently selected from hydrogen, (1-6 C)alkyl or phenyl; and wherein Rₕ and Rⱼ are each independently selected from hydrogen, (1 -6 C)alkyl or phenyl or Rₕ and R together with the nitrogen atom to which they are attached form a 3-7 membered ring which may optionally include further heteroatoms and is optionally further substituted by one or more substituents selected from halo, trifluoromethyl, trifluoromethoxy, cyano, hydroxyl, carboxyl, carbamoyl, sulphamoyl, and (1-2 C)alkyl; and
   (ii) any (1-4 C)alkyl, (3-7 C)cycloalkyl, heterocyclyl, heteroaryl or aryl in a R₁₀₀ substituent group is optionally further substituted by one or more substituents selected from halo, trifluoromethyl, trifluoromethoxy, cyano, hydroxyl, (1 -2 C)alkyl, (1-2 C)haloalkyl, (1-2 C)hydroxyalkyl, ORₖ, C(O)Rₖ, C(O)ORₖ, OC(O)Rₖ, C(O)N(Rₗ )Rₖ, N(Rₗ )C(O)Rₖ, S(O)_{y2} Rₖ, SO₂ N(Rₗ )Rₖ, N(Rⱼ )SO₂ Rₖ, or NRₗ Rₖ, wherein Rₖ and Rₗ are selected from hydrogen or (1-2 C)alkyl; R₂ is hydrogen; R₃ is selected from hydrogen, (1-8 C)alkyl, (2-8 C)alkenyl, (2-8 C)alkynyl, (3-7 C)cycloalkyl, aryl, heteroaryl, heterocyclyl, (CH₂ )₀₋₃ (3-7 C)cycloalkyl, (CH₂ )₀₋₃ heterocyclyl, (CH₂ )₀₋₃ heteroaryl, (CH₂ )₀₋₃ aryl, —C(O)—(CH₂ )₀₋₃ (3-7 C)cycloalkyl, —C(O)—(CH₂ )₀₋₃ heterocyclyl, —C(O)—(CH₂ )₀₋₃ heteroaryl, —C(O)—(CH₂ )₀₋₃ aryl or —C(O)O(1-8 C)alkyl, —C(O)NR₃ₐ-(1-8 C)alkyl, —C(O)NR₃ₐ—(CH₂ )₀₋₃ (3-7 C)cycloalkyl, —C(O)NR₃ₐ—(CH₂ )₀₋₃ heterocyclyl, —C(O)NR₃ₐ—(CH₂ )₀₋₃ heteroaryl, —C(O)NR₃ₐ—(CH₂ )₀₋₃ aryl;
   wherein R₃ₐ is hydrogen or (1-2 C)alkyl;
   wherein any (1-8 C)alkyl, (2-8 C)alkenyl, (2-8 C)alkynyl, (3-7 C)cycloalkyl, aryl, heteroaryl and heterocyclyl moiety is optionally substituted by one or more R₂₀₀ substituents;
   wherein R₂₀₀ is selected from halo, (1-2 C)haloalkyl (e.g. trifluoromethyl), (1 -2 C)haloalkoxy (e.g. trifluoromethoxy), cyano, hydroxyl, nitro, (1-4 C)alkyl, (1-4 C)hydroxyalkyl, (CH₂ )_{z}ORₘ, (CH₂ )_{z} C(O)Rₘ, (CH₂ )_{z} C(O)ORₘ, (CH₂ )_{z}OC(O)Rₘ, (CH₂ )_{z} C(O)N(Rₒ )Rₚ, (CH₂ )_{z} N(Rn)C(O)Rₘ, (CH₂ )_{z} N(Rn)C(O)ORₘ, (CH₂ )_{z} S(O)_{y3} Rₘ, (CH₂ )_{z} SO₂ N(Rₒ )Rₚ, (CH₂ )_{z}N(Rₙ )SO₂ Rₘ, (CH₂ )_{z} NRₒ Rₚ, (CH₂ )_{z} (3-7 C)cycloalkyl, (CH₂ )_{z} heterocyclyl, (CH₂ )_{z}heteroaryl, (CH₂ )_{z} aryl;
   and wherein:
   (i) Rₘ and Rₙ are each independently selected from hydrogen, (1-6 C)alkyl or (CH₂ )₀₋₃ phenyl; Rₒ and Rₚ are each independently selected from hydrogen, (1-6 C)alkyl or phenyl or Rₒ and Rₚ together with the nitrogen atom to which they are attached form a 3-7 membered ring which may optionally include further heteroatoms, and wherein any 3-7 membered ring formed Rₒ and Rₚ, and any alkyl or phenyl group present for Rₘ, Rₙ, Rₒ and Rₚ is optionally further substituted by one or more substituents selected from halo, trifluoromethyl, trifluoromethoxy, cyano, hydroxyl, carboxyl, carbamoyl, sulphamoyl, and (1 -2 C)alkyl; and
   (ii) any (3-7 C)cycloalkyl, heterocyclyl, heteroaryl or aryl moiety in a R₂₀₀ substituent group is optionally further substituted by one or more substituents selected from halo, trifluoromethyl, trifluoromethoxy, cyano, hydroxyl, (1-2 C)alkyl, (1 -2 C)haloalkyl, (1-2 C)hydroxyalkyl, OR_{q}, C(O)R_{q}, C(O)OR_{q}, OC(O)R_{q}, C(O)N(R_{q} )Rᵣ, N(Rᵣ )C(O)R_{q}, S(O)_{y4} R_{q}, SO₂ N(Rᵣ )R_{q}, N(Rᵣ )SO₂ R_{q}, or NRᵣ R_{q}, wherein R_{q} is hydrogen, (1-2 C)alkyl or phenyl, and Rᵣ are selected from hydrogen or (1-2 C)alkyl;
or R₂ and R₃ are linked such that together they form a —X₂ ═CQ- group;
X₂ is selected from N and CRₐ ; wherein Rₐ is selected from hydrogen, fluoro, chloro, methyl, cyano, difluoromethyl, and trifluoromethyl; and
Q is hydrogen, halo, cyano or a group of the formula:

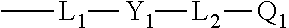

wherein:
   L₁ is absent or (1-4 C)alkylene;
   Y₁ is absent or O, S, SO, SO₂, N(R_{y1} ), C(O), C(O)O, OC(O), C(O)N(R_{y1} ), or N(R_{y1} )C(O), wherein R_{y1} is selected from hydrogen or (1-6 C)alkyl;
   L₂ is absent or (1-3 C)alkylene; and
   Q₁ is hydrogen, (1-6 C)alkyl, (2-6 C)alkenyl, (2-6 C)alkynyl, aryl, (3-8 C)cycloalkyl, heteroaryl or heterocyclyl;
   wherein Q is optionally further substituted by one or more substituent groups independently selected from oxo, (1-6 C)alkyl, halo, (1-4 C)haloalkyl, (1 -4 C)haloalkoxy, (1-4 C)aminoalkyl, (1-4 C)hydroxyalkyl, cyano, or by one or more group(s) of the formula:

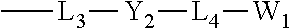

wherein:
   L₃ is absent or (1-4 C)alkylene;
   Y₂ is absent or selected from or O, S, SO, SO₂, N(R_{y2} ), C(O), C(O)O, OC(O), C(O)N(R_{y2} ), N(R_{y2} )C(O) or S(O)₂ N(R_{y2} ), N(R_{y2} )SO₂ wherein R_{y2} is selected from hydrogen or (1-4 C)alkyl;
   L₄ is absent or (1-3 C)alkylene; and
   W₁ is hydrogen, (1-6 C)alkyl, aryl, (3-8 C)cycloalkyl, heteroaryl or heterocyclyl;
   wherein W₁ is optionally substituted by one or more substituents selected from oxo, (1-4 C)alkyl, halo, (1-4 C)haloalkyl, (1-4 C)haloalkoxy, (1 -4 C)alkoxy, amino, (1-4 C)alkylamino, di[(1-4 C)alkyl]amino C(O)OH, C(O)O(1-4 C)alkyl, (CH₂ )₀₋3 -heterocyclyl or cyano;
R₄ is selected from hydrogen or halo;
X₁ is N or CR₅, wherein R₅ is selected from hydrogen, halo, cyano, or amino;
x is independently selected from 0, 1, 2 or 3;
y1, y2, y3 and y4 are each independently selected from 0, 1 or 2;
z is independently selected from 0, 1, 2 or 3;
   with the proviso that:
   X₁ and X₂ are only N when Q is hydrogen;
   when X₁ and X₂ are CR₅ or CRₐ, Q is not hydrogen;
   Q₁ is not hydrogen when L₁, Y₁, and L₂ are all absent;
   and at least one of R₁, Q, Rₐ, R₄ or R₅ is a substituent other than hydrogen.

Particular compounds of the invention include, for example, compounds of the Formula (I), or pharmaceutically acceptable salts, hydrates and/or solvates thereof, defined herein having one of the structural formulae (Ia), (Ib) or (Ic) shown below:

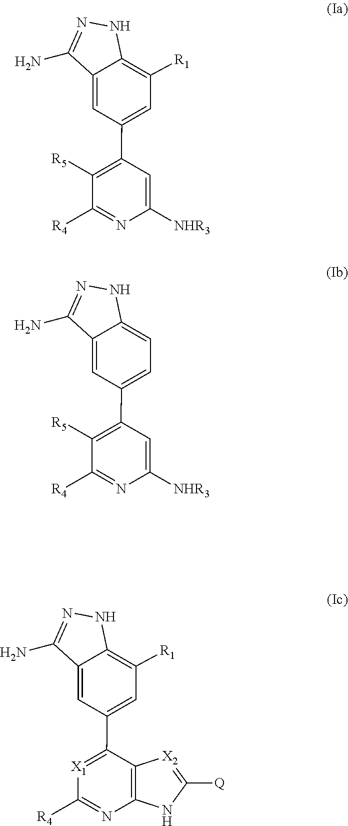

wherein R₁, R₃, R₄, R₅, Q, X₁ and X₂ are each as defined herein.

Particular compounds of the invention include, for example, compounds of the Formula (I) [including sub-formulae (Ia), (Ib) or (Ic), or pharmaceutically acceptable salts, hydrates and/or solvates thereof, wherein, unless otherwise stated, each of R₁, R₂, R₃, R₄, X₁ and any associated substituent groups has any of the meanings defined hereinbefore or in any of paragraphs (1 ) to (49 ) hereinafter:
(1 ) R₁ is selected from hydrogen, halogen, (2-6 C)alkynyl, (3-7 C)cycloalkyl, phenyl, a 5 or 6 -membered heteroaryl or a 4 to 7 -membered heterocyclyl,
   wherein said (2-6 C)alkynyl, (3-7 C)cycloalkyl, phenyl, heteroaryl and heterocyclyl are optionally substituted by one or more R₁₀₀ substituents;
   and wherein each R₁₀₀ is independently selected from halo, (1-2 C)haloalkyl (e.g. trifluoromethyl) (1-2 C)haloalkoxy e.g. trifluoromethoxy, cyano, hydroxyl, (1-4 C)alkyl, (1 -4 C)hydroxyalkyl, (CH₂ )_{z}OR_{f}, (CH₂ )_{z} C(O)R_{f}, (CH₂ )_{z} C(O)OR_{f}, (CH₂ )_{z}OC(O)R_{f}, (CH₂ )_{z} C(O)N(R;)Rₙ, (CH₂ )_{z} N(R_{g} )C(O)R_{f}, (CH₂ )_{z} S(O)_{y1} R_{f}, (CH₂ )_{z} SO₂ N(Rⱼ )Rₙ, (CH₂ )_{z} N(R_{g} )SO₂ R_{f}, (CH₂ )_{z} NRⱼ Rₕ, (CH₂ )_{z} (3-7 C)cycloalkyl, (CH₂ )_{z} heterocyclyl, (CH₂ )_{z} heteroaryl (CH₂ )_{z} aryl;
   and wherein:
   (i) R_{f} and R_{g} are each independently selected from hydrogen or (1-2 C)alkyl; and wherein Rₕ and Rⱼ are each independently selected from hydrogen or (1 -2 C)alkyl or Rₕ and Rⱼ together with the nitrogen atom to which they are attached form a 3-7 membered ring which may optionally include further heteroatoms; and
   any (1-4 C)alkyl, (3-7 C)cycloalkyl, heterocyclyl, heteroaryl or aryl moiety in a R₁₀₀ substituent group is optionally further substituted by one or more substituents selected from halo, trifluoromethyl, trifluoromethoxy, cyano, hydroxyl, (1-2 C)alkyl, (1 -2 C)haloalkyl, (1-2 C)hydroxyalkyl, ORₖ, C(O)Rₖ, C(O)ORₖ, OC(O)Rₖ, C(O)N(Rₗ)Rₖ, N(Rₗ )C(O)Rₖ, S(O)_{y2} Rₖ, SO₂ N(Rₗ )Rₖ, N(Rₗ )SO₂ Rₖ, or NRₗ Rₖ, wherein Rₖ and Rₗ are selected from hydrogen or (1-2 C)alkyl.
(2 )Rₗ is selected from hydrogen, halogen, (2-6 C)alkynyl, phenyl or a 5 or 6 -membered heteroaryl,
   wherein said (2-6 C)alkynyl, phenyl or heteroaryl are optionally substituted by one or more R₁₀₀ substituents;
   and wherein each R₁₀₀ is independently selected from halo, (1-2 C)haloalkyl (e.g. trifluoromethyl), (1-2 C)haloalkoxy e.g. (trifluoromethoxy), cyano, hydroxyl, (1-4 C)alkyl, (1-4 C)hydroxyalkyl, (CH₂ )ₓOR_{f}, (CH₂ )ₓ C(O)R_{f}, (CH₂ )ₓ C(O)OR_{f}, (CH₂ )ₓOC(O)R_{f}, (CH₂ )ₓ C(O)N(R,)Rₕ, (CH₂ )ₓ N(R_{g} )C(O)Rᵣ, (CH₂ )ₓ S(O)_{y1} R_{f}, (CH₂ )ₓ SO₂ N(Rⱼ )Rₕ, (CH₂ )ₓ N(R_{g} )SO₂ Rᵣ, (CH₂ )ₓ NRₗ Rₕ, (CH₂ )ₓ (3-7 C)cycloalkyl, (CH₂ )ₓ -[4-6 membered heterocyclyl], (CH₂ )ₓ -[5 or 6 membered heteroaryl] or (CH₂ )xphenyl;
   and wherein:
   (i) R_{f} and R_{g} are each independently selected from hydrogen or (1-2 C)alkyl; and wherein Rₕ and Rⱼ are each independently selected from hydrogen or (1 -2 C)alkyl or Rₕ and Rⱼ together with the nitrogen atom to which they are attached form a 3-7 membered ring which may optionally include further heteroatoms; and
   any (1-4 C)alkyl, (3-7 C)cycloalkyl, heterocyclyl, heteroaryl or aryl moiety in a R₁₀₀ substituent group is optionally further substituted by one or more substituents selected from halo, trifluoromethyl, trifluoromethoxy, cyano, hydroxyl, (1-2 C)alkyl, (1 -2 C)haloalkyl, (1-2 C)hydroxyalkyl ORₖ, C(O)Rₖ, C(O)ORₖ, OC(O)Rₖ, C(O)N(R,)Rₖ, N(Rₗ )C(O)Rₖ, S(O)_{y2} Rₖ, SO₂ N(Rₗ )Rₖ, N(Rₗ )SO₂ Rₖ, or NRₗ Rₖ, wherein Rₖ and R, are selected from hydrogen or (1-2 C)alkyl.
(3 )Rₗ is selected from hydrogen, halogen, (2-6 C)alkynyl, phenyl or a 5 or 6 -membered heteroaryl,
   wherein said (2-6 C)alkynyl, phenyl or heteroaryl are optionally substituted by one or more R₁₀₀ substituents;
   and wherein each R₁₀₀ is independently selected from halo, trifluoromethyl, trifluoromethoxy, cyano, hydroxyl, (1-4 C)alkyl, (1-4 C)hydroxyalkyl, (CH₂ )ₓOR_{f}, (CH₂ )ₓ C(O)R_{f}, (CH₂ )ₓ C(O)OR_{f}, (CH₂ )ₓOC(O)R_{f}, (CH₂ )ₓ C(O)N(Rⱼ )Rₙ, (CH₂ )ₓ N(R_{g} )C(O)R_{f}, (CH₂ )ₓ S(O)_{y1} R_{f}, (CH₂ )ₓ SO₂ N(Rⱼ )Rᵣ, (CH₂ )ₓ N(R_{g} )SO₂ R_{f}, (CH₂ )ₓ NRⱼ Rₕ, (CH₂ )ₓ (3 -7 C)cycloalkyl, (CH₂ )ₓ -[4-6 membered heterocyclyl], (CH₂ )ₓ -[5 or 6 membered heteroaryl] or (CH₂ )xphenyl;
   and wherein:
   (i) R_{f} and R_{g} are each independently selected from hydrogen or (1-2 C)alkyl; and wherein Rₕ and Rⱼ are each independently selected from hydrogen or (1 -2 C)alkyl; and
   any (1-4 C)alkyl, (3-7 C)cycloalkyl, heterocyclyl, heteroaryl or aryl moiety in a R₁₀₀ substituent group is optionally further substituted by one or more substituents selected from halo, trifluoromethyl, trifluoromethoxy, cyano, hydroxyl, (1-2 C)alkyl, (1 -2 C)haloalkyl, (1-2 C)hydroxyalkyl or ORₖ, wherein Rₖ is selected from hydrogen or (1 -2 C)alkyl.
(4 )Rₗ is selected from hydrogen, halogen, (2-6 C)alkynyl, phenyl or a 5 or 6 -membered heteroaryl,
   wherein said (2-6 C)alkynyl, phenyl or heteroaryl are optionally substituted by one or more R₁₀₀ substituents;
   and wherein each R₁₀₀ is independently selected from halo, trifluoromethyl, trifluoromethoxy, cyano, hydroxyl, (1-4 C)alkyl, (1-4 C)hydroxyalkyl, (CH₂ )ₓOR_{f}, C(O)Rᵣ, C(O)OR_{f}, OC(O)R_{f}, C(O)N(R)Rₕ, N(R_{g} )C(O)R_{f}, S(O)_{y1} R_{f}, SO₂ N(R)Rₙ, N(R_{g} )SO₂ R_{f}, NR₁ Rₕ, (CH₂ )ₓ -[4-6 membered heterocyclyl], or (CH₂ )ₓ phenyl;
   and wherein:
   (i) R_{f} and R_{g} are each independently selected from hydrogen or (1-2 C)alkyl; and
wherein Rₕ and Rⱼ are each independently selected from hydrogen or (1 -2 C)alkyl; and
any (1-4 C)alkyl, (3-7 C)cycloalkyl, heterocyclyl, heteroaryl or aryl moiety in a R₁₀₀ substituent group is optionally further substituted by one or more substituents selected from halo, trifluoromethyl, trifluoromethoxy, cyano, hydroxyl, (1-2 C)alkyl, (1-2 C)haloalkyl, (1 -2 C)hydroxyalkyl or ORₖ, wherein Rₖ is selected from hydrogen or (1-2 C)alkyl.
(5 )Rₗ is selected from:
   (i) hydrogen or halogen;
   (ii) ethynyl, i.e.

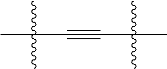 [SMILES: *C#C*]

which is optionally substituted by R₁₀₀;
(ii) phenyl, which is optionally substituted by R₁₀₀ ;
(iii) a 5 or 6 -membered heteroaryl, which is optionally substituted by R₁₀₀ ;
   and wherein each R₁₀₀ is independently selected from halo, trifluoromethyl, trifluoromethoxy, cyano, hydroxyl, (1-4 C)alkyl, (1-4 C)hydroxyalkyl, (CH₂ )ₓOR_{f}, C(O)R_{f}, C(O)OR_{f}, OC(O)R_{f}, C(O)N(Rⱼ )Rₕ, N(R_{g} )C(O)R_{f}, S(O)_{y1} R_{f}, SO₂ N(Rⱼ )Rₕ, N(R_{g} )SO₂ R_{f}, NRⱼ Rₕ, (CH₂ )ₓ -[4-6 membered heterocyclyl], or (CH₂ )ₓ phenyl;
   and wherein:
   (i) R_{f} and R_{g} are each independently selected from hydrogen or (1-2 C)alkyl; and
   wherein Rₕ and Rⱼ are each independently selected from hydrogen or (1 -2 C)alkyl; and
(ii) any (1-4 C)alkyl, (3-7 C)cycloalkyl, heterocyclyl, heteroaryl or aryl moiety in a R₁₀₀ substituent group is optionally further substituted by one or more substituents selected from halo, trifluoromethyl, trifluoromethoxy, cyano, hydroxyl, (1 -2 C)alkyl, (1-2 C)haloalkyl, (1-2 C)hydroxyalkyl or ORₖ, wherein Rₖ is selected from hydrogen or (1-2 C)alkyl.
(6 ) R₁ is selected from:
   (i) hydrogen or halogen;
   (ii) ethynyl, i.e.

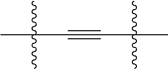 [SMILES: *C#C*]

which is optionally substituted by R₁₀₀ ;
(iii) phenyl, which is optionally substituted by R₁₀₀ ;
and wherein each R₁₀₀ is independently selected from halo, trifluoromethyl, trifluoromethoxy, cyano, hydroxyl, (1-4 C)alkyl, (1-4 C)hydroxyalkyl, (CH₂ )ₓOR_{f}, C(O)R_{f}, C(O)OR_{f}, OC(O)R_{f}, C(O)N(Rⱼ )Rₕ, N(R_{g} )C(O)R_{f}, S(O)_{y1} R_{f}, SO₂ N(Rⱼ )Rₕ, N(R_{g} )SO₂ R_{f}, NRⱼ Rₕ, (CH₂ )ₓ -[4-6 membered heterocyclyl], or (CH₂ )ₓ phenyl;
and wherein:
(i) R_{f} and R_{g} are each independently selected from hydrogen or (1-2 C)alkyl; and wherein Rₕ and Rⱼ are each independently selected from hydrogen or (1 -2 C)alkyl; and
any (1-4 C)alkyl, (3-7 C)cycloalkyl, heterocyclyl, heteroaryl or aryl moiety in a R₁₀₀ substituent group is optionally further substituted by one or more substituents selected from halo, trifluoromethyl, trifluoromethoxy, cyano, hydroxyl, (1-2 C)alkyl, (1-2 C)haloalkyl, (1 -2 C)hydroxyalkyl or ORₖ, wherein Rₖ is selected from hydrogen or (1-2 C)alkyl.
(7 ) Rₐ is selected from:
   (i) hydrogen or halogen;
   (ii) ethynyl, i.e.

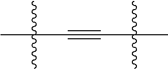 [SMILES: *C#C*]

which is optionally substituted by R₁₀₀ ;
(iii) phenyl, which is optionally substituted by R₁₀₀ ;
and wherein each R₁₀₀ is independently selected from halo, trifluoromethyl, trifluoromethoxy, cyano, hydroxyl, (1-4 C)alkyl, (1-4 C)hydroxyalkyl, (CH₂ )ₓOR_{f}, C(O)R_{f}, C(O)N(Rⱼ )Rₕ, S(O)_{y1} R_{f}, SO₂ N(Rⱼ )Rₕ, N(R_{g} )SO₂ R_{f}, NRⱼ Rₕ, (CH₂ )ₓ -[4-6 membered heterocyclyl], or (CH₂ )ₓ phenyl;
and wherein:
(ii) Rᵣ and R_{g} are each independently selected from hydrogen or (1-2 C)alkyl; and wherein Rₕ and Rⱼ are each independently selected from hydrogen or (1 -2 C)alkyl.
(8 ) R₁ is selected from:
   (i) hydrogen or halogen;
   (ii) ethynyl, i.e.

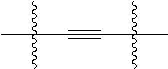 [SMILES: *C#C*]

which is optionally substituted by R₁₀₀ ;
(iii) phenyl, which is optionally substituted by R₁₀₀ ;
and wherein each R₁₀₀ is independently selected from halo, trifluoromethyl, trifluoromethoxy, cyano, hydroxyl or (1-4 C)alkyl.
(9 ) R₃ is selected from hydrogen, (1-8 C)alkyl, (2-8 C)alkenyl, (2-8 C)alkynyl, (3 -7 C)cycloalkyl, phenyl, a 5 or 6 -membered heteroaryl, a 4 to 7 -membered heterocyclyl, (CH₂ )₀₋₃ (3-7 C)cycloalkyl, (CH₂ )₀₋₃[4 to 7 -membered heterocyclyl], (CH₂ )₀₋₃[5 or 6 -membered heteroaryl], (CH₂ )₀₋₃phenyl, —C(O)—(CH₂ )₀₋₃ (3-7 C)cycloalkyl, —C(O)—(CH₂ )₀₋₃[4 to 7 -membered heterocyclyl], —C(O)—(CH₂ )₀ -₃[5 or 6 -membered heteroaryl], —C(O)—(CH₂ )₀₋₃phenyl, —C(O)O(1-8 C)alkyl, —C(O)NR₃ₐ-(1-8 C)alkyl, —C(O)NR₃ₐ—(CH₂ )₀₋₃ (3 -7 C)cycloalkyl, —C(O)NR₃ₐ—(CH₂ )₀₋₃[5 to 7 -membered heterocyclyl], —C(O)NR₃ₐ—(CH₂ )₀₋₃[5 or 6 -membered heteroaryl], or —C(O)NR₃ₐ—(CH₂ )₀₋3 phenyl;
   wherein R₃ₐ is hydrogen or (1-2 C)alkyl;
   wherein any (1-8 C)alkyl, (2-8 C)alkenyl, (2-8 C)alkynyl, (3-7 C)cycloalkyl, phenyl, 5 or 6 -membered heteroaryl or 4 to 7 -membered heterocyclyl moiety is optionally substituted by one or more R₂₀₀ substituents;
   wherein R₂₀₀ is selected from halo, (1-2 C)haloalkyl (e.g. trifluoromethyl), (1 -2 C)haloalkoxy (e.g. trifluoromethoxy), cyano, hydroxyl, nitro, (1-4 C)alkyl, (1 -4 C)hydroxyalkyl, (CH₂ )_{z}ORₘ, (CH₂ )_{z} C(O)Rₘ, (CH₂ )_{z} C(O)ORₘ, (CH₂ )_{z} C(O)N(Rₒ )Rₚ, (CH₂ )_{z} N(Rₙ )C(O)Rₘ, (CH₂ )_{z} N(Rₙ )C(O)ORₘ, (CH₂ )_{z} S(O)_{y3} Rₘ, (CH₂ )_{z} SO₂ N(Rₒ )Rₚ, (CH₂ )_{z} N(Rₙ )SO₂ Rₘ, (CH₂ )_{z} NRₒ Rₚ, (CH₂ )_{z} (3-7 C)cycloalkyl, (CH₂ )_{z} [4 to 7 -membered heterocyclyl], (CH₂ )_{z}[5 or 6 -membered heteroaryl, (CH₂ )_{z} phenyl;
   and wherein:
   (i) Rₘ and Rₙ are each independently selected from hydrogen, (1-6 C)alkyl or (CH₂ )₀₋₃ phenyl; Rₗ and Rₚ are each independently selected from hydrogen, (1-6 C)alkyl or phenyl or Rₒ and Rₚ together with the nitrogen atom to which they are attached form a 3-7 membered ring which may optionally include further heteroatoms, and wherein any 3-7 membered ring formed Rₒ and Rₚ, and any alkyl or phenyl group present for Rₘ, Rₙ, Rₒ and Rₚ is optionally further substituted by one or more substituents selected from halo, trifluoromethyl, trifluoromethoxy, cyano, hydroxyl, carboxyl, carbamoyl, sulphamoyl, and (1 -2 C)alkyl; and
   any (3-7 C)cycloalkyl, heterocyclyl, heteroaryl or phenyl moiety in a R₂₀₀ substituent group is optionally further substituted by one or more substituents selected from halo, trifluoromethyl, trifluoromethoxy, cyano, hydroxyl, (1-2 C)alkyl, (1-2 C)haloalkyl, (1 -2 C)hydroxyalkyl, OR_{q}, C(O)R_{q}, C(O)OR_{q}, OC(O)R_{q}, C(O)N(R_{q} )Rᵣ, N(R,)C(O)R_{q}, S(O)_{y4} R_{q}, SO₂ N(Rᵣ )R_{q}, N(R,)SO₂ R_{q}, or NRᵣ R_{q}, wherein R_{q} is hydrogen, (1-2 C)alkyl or phenyl, and Rᵣ are selected from hydrogen or (1-2 C)alkyl.
(10 ) R₃ is selected from hydrogen, (1-8 C)alkyl, (2-8 C)alkenyl, (2-8 C)alkynyl, (3 -7 C)cycloalkyl, phenyl, a 5 or 6 -membered heteroaryl, a 4 to 7 -membered heterocyclyl, (CH₂ )₀₋₃ (3-7 C)cycloalkyl, (CH₂ )₀₋₃[4 to 7 -membered heterocyclyl], (CH₂ )₀₋₃ [5 or 6 -membered heteroaryl], (CH₂ )₀₋₃phenyl, —C(O)—(CH₂ )₀₋₃ (3-7 C)cycloalkyl, —C(O)—(CH₂ )₀₋₃[4 to 7 -membered heterocyclyl], —C(O)—(CH₂ )₀ -₃[5 or 6 -membered heteroaryl], —C(O)—(CH₂ )₀₋₃phenyl, —C(O)O(1-8 C)alkyl, —C(O)NR₃ₐ-(1-8 C)alkyl, —C(O)NR₃ₐ—(CH₂ )₀₋₃ (3 -7 C)cycloalkyl, —C(O)NR₃ₐ—(CH₂ )₀₋₃[5 to 7 -membered heterocyclyl], —C(O)NR₃ₐ—(CH₂ )₀₋₃[5 or 6 -membered heteroaryl], or —C(O)NH—(CH₂ )₀₋₃phenyl;
   wherein R₃ₐ is hydrogen or (1-2 C)alkyl;
   wherein any (1-8 C)alkyl, (2-8 C)alkenyl, (2-8 C)alkynyl, (3-7 C)cycloalkyl, phenyl, 5 or 6 -membered heteroaryl or 4 to 7 -membered heterocyclyl moiety is optionally substituted by one or more R₂₀₀ substituents;
   wherein R₂₀₀ is selected from halo, trifluoromethyl, trifluoromethoxy, cyano, hydroxyl, nitro, (1-4 C)alkyl, (1-4 C)hydroxyalkyl, (CH₂ )_{z}ORₘ, (CH₂ )_{z} C(O)Rₘ, (CH₂ )_{z} C(O)ORₘ, (CH₂ )_{z} C(O)N(Rₒ )Rₚ, (CH₂ )_{z} N(Rₙ )C(O)Rₘ, (CH₂ )_{z} N(R,)C(O)ORₘ, (CH₂ )_{z} S(O)_{y3} Rₘ, (CH₂ )_{z} SO₂ N(Rₒ )Rₚ, (CH₂ )_{z} N(Rₙ )SO₂ Rₘ, (CH₂ )_{z} NRₒ Rₚ, (CH₂ )_{z} (3-7 C)cycloalkyl, (CH₂ )_{z} [4 to 7 -membered heterocyclyl], (CH₂ )_{z} [5 or 6 -membered heteroaryl, (CH₂ )_{z} phenyl;
   and wherein:
   (i) Rₘ and Rₙ are each independently selected from hydrogen, (1-6 C)alkyl or (CH₂ )₀₋₃ phenyl; Rₒ and Rₚ are each independently selected from hydrogen, (1-6 C)alkyl or phenyl or Rₒ and Rₚ together with the nitrogen atom to which they are attached form a 3-7 membered ring which may optionally include further heteroatoms, and wherein any 3-7 membered ring formed Rₒ and Rₚ, and any alkyl or phenyl group present for Rₘ, Rₙ, Rₒ and Rₚ is optionally further substituted by one or more substituents selected from halo, trifluoromethyl, trifluoromethoxy, cyano, hydroxyl, carboxyl, carbamoyl, sulphamoyl, and (1 -2 C)alkyl.
(11 ) R₃ is selected from hydrogen, (1-8 C)alkyl, (2-8 C)alkenyl, (2-8 C)alkynyl, (3 -7 C)cycloalkyl, phenyl, a 5 or 6 -membered heteroaryl, a 4 to 7 -membered heterocyclyl, (CH₂ )₀₋₃ (3-7 C)cycloalkyl, (CH₂ )₀₋₃[4 to 7 -membered heterocyclyl], (CH₂ )₀₋₃[5 or 6 -membered heteroaryl], (CH₂ )₀₋₃phenyl, —C(O)—(CH₂ )₀₋₃ (3-7 C)cycloalkyl, —C(O)—(CH₂ )₀ -₃[4 to 7 -membered heterocyclyl], —C(O)—(CH₂ )₀₋₃[5 or 6 -membered heteroaryl], —C(O)—(CH₂ )₀₋₃ phenyl, —C(O)O(1-8 C)alkyl, —C(O)NR₃ₐ-(1-8 C)alkyl, —C(O)NR₃ₐ—(CH₂ )₀₋₃ (3-7 C)cycloalkyl, —C(O)NR₃ₐ—(CH₂ )₀₋₃[5 to 7 -membered heterocyclyl], —C(O)NR₃ₐ—(CH₂ )₀₋₃[5 or 6 -membered heteroaryl], or —C(O)NR₃ₐ—(CH₂ )₀₋₃phenyl;
   wherein R₃ₐ is hydrogen or (1-2 C)alkyl;
   wherein any (1-8 C)alkyl, (2-8 C)alkenyl, (2-8 C)alkynyl, (3-7 C)cycloalkyl, phenyl, 5 or 6 -membered heteroaryl or 4 to 7 -membered heterocyclyl moiety is optionally substituted by one or more R₂₀₀ substituents;
   wherein R₂₀₀ is selected from halo, trifluoromethyl, trifluoromethoxy, cyano, hydroxyl, nitro, (1-4 C)alkyl, (1-4 C)hydroxyalkyl, (CH₂ )_{z}ORₘ, (CH₂ )_{z} C(O)Rₘ, (CH₂ )_{z} C(O)ORₘ, (CH₂ )_{z} C(O)N(Rₒ )Rₚ, (CH₂ )_{z} N(Rₙ )C(O)Rₘ, (CH₂ )_{z} N(Rₙ )C(O)ORₘ, (CH₂ )_{z} S(O)_{y3} Rₘ, (CH₂ )_{z} SO₂ N(Rₒ )Rₚ, (CH₂ )_{z} N(R,)SO₂ Rₘ, or (CH₂ )_{z}NRoRₚ ;
   and wherein:
   Rₘ and Rₙ are each independently selected from hydrogen, (1-6 C)alkyl or phenyl; Rₒ and Rₚ are each independently selected from hydrogen, (1-6 C)alkyl or (CH₂ )₀₋₂ phenyl or Rₒ and Rₚ together with the nitrogen atom to which they are attached form a 3-7 membered ring which may optionally include further heteroatoms, and wherein any 3-7 membered ring formed Rₒ and Rₚ, and any alkyl or phenyl group present for Rₘ, Rₙ, Rₒ and Rₚ is optionally further substituted by one or more substituents selected from halo, trifluoromethyl, trifluoromethoxy, cyano, hydroxyl, carboxyl, carbamoyl, sulphamoyl, and (1-2 C)alkyl.
(12 ) R₃ is selected from hydrogen, (1-8 C)alkyl, (2-8 C)alkenyl, (2-8 C)alkynyl, (3 -7 C)cycloalkyl, phenyl, a 5 or 6 -membered heteroaryl, a 4 to 7 -membered heterocyclyl, (CH₂ )₀₋₃ (3-7 C)cycloalkyl, (CH₂ )₀₋₃[4 to 7 -membered heterocyclyl], (CH₂ )₀₋₃[5 or 6 -membered heteroaryl], (CH₂ )₀₋3 phenyl, —C(O)—(CH₂ )₀₋₃ (3-7 C)cycloalkyl, —C(O)—(CH₂ )₀₋₃[5 or 6 -membered heteroaryl], —C(O)—(CH₂ )₀₋₃phenyl, —C(O)O(1-8 C)alkyl, —C(O)NR₃ₐ-(1 -8 C)alkyl, —C(O)NR₃ₐ—(CH₂ )₀₋₃ (3-7 C)cycloalkyl, —C(O)NR₃ₐ—(CH₂ )₀₋₃[5 or 6 -membered heteroaryl], or —C(O)NR₃ₐ—(CH₂ )₀₋₃phenyl;
   wherein R₃ₐ is hydrogen or (1-2 C)alkyl;
   wherein any (1-8 C)alkyl, (2-8 C)alkenyl, (2-8 C)alkynyl, (3-7 C)cycloalkyl, phenyl, 5 or 6 -membered heteroaryl or 4 to 7 -membered heterocyclyl moiety is optionally substituted by one or more R₂₀₀ substituents;
   wherein R₂₀₀ is selected from halo, trifluoromethyl, trifluoromethoxy, cyano, hydroxyl, nitro, (1-4 C)alkyl, (1-4 C)hydroxyalkyl, (CH₂ )_{z}ORₘ, (CH₂ )_{z} C(O)Rₘ, (CH₂ )_{z} C(O)ORₘ, (CH₂ )_{z} C(O)N(Rₒ )Rₚ, (CH₂ )_{z} N(Rₙ )C(O)Rₘ, (CH₂ )_{z} N(Rₙ )C(O)ORₘ, (CH₂ )_{z} S(O)_{y3} Rₘ, (CH₂ )_{z} SO₂ N(Rₒ )Rₚ, (CH₂ )_{z} N(Rₙ )SO₂ Rₘ, or (CH₂ )_{z} NRₒ Rₚ ;
   and wherein:
   Rₘ and Rₙ are each independently selected from hydrogen, (1-6 C)alkyl or phenyl; R_{c} and Rₚ are each independently selected from hydrogen, (1-6 C)alkyl or (CH₂ )₀₋₂ phenyl or Rₒ and Rₚ together with the nitrogen atom to which they are attached form a 3-7 membered ring which may optionally include further heteroatoms, and wherein any 3-7 membered ring formed Rₒ and Rₚ, and any alkyl or phenyl group present for Rₘ, Rₙ, Rₒ and Rₚ is optionally further substituted by one or more substituents selected from halo, trifluoromethyl, trifluoromethoxy, cyano, hydroxyl, carboxyl, carbamoyl, sulphamoyl, and (1-2 C)alkyl.
(13 ) R₃ is selected from hydrogen or a group of the formula:

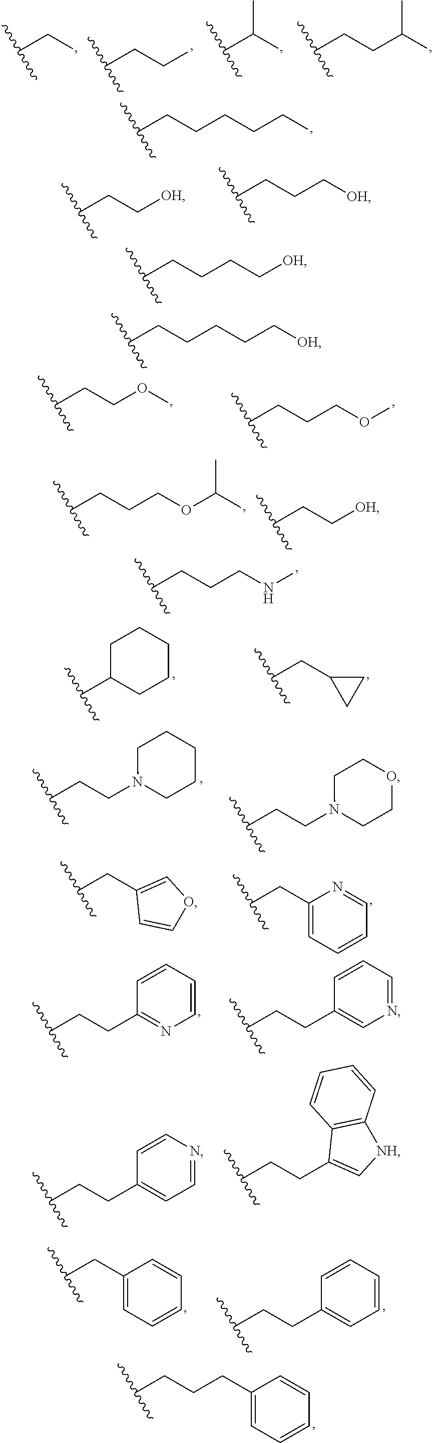

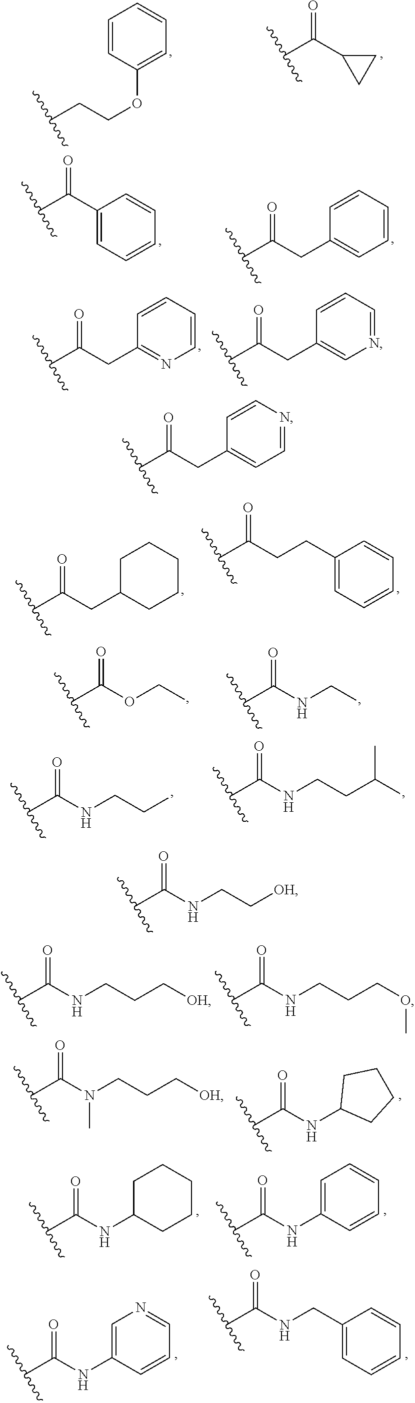

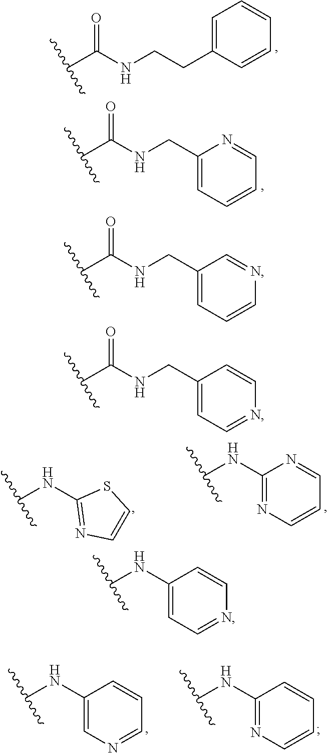

wherein any cycloalkyl, heterocyclyl, aryl or heteroaryl group above is optionally substituted by one or more R₂₀₀ substituents, wherein R₂₀₀ is as defined herein.
(14 ) R₂ and R₃ are linked such that together they form a —X₂ ═CQ- group;
(15 ) X₂ is selected from N and CRₐ ; wherein Rₐ is selected from hydrogen, fluoro, chloro, methyl, or cyano.
(16 ) X₂ is selected from N and CRₐ ; wherein Rₐ is selected from hydrogen, fluoro, chloro or methyl.
(17 ) X₂ is selected from N and CRₐ ; wherein Rₐ is selected from hydrogen, fluoro or chloro.
(18 ) Q is hydrogen, halo, cyano or a group of the formula:

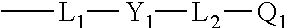

wherein:
   L₁ is absent or (1-4 C)alkylene;
   Y₁ is absent or O, S, SO, SO₂, N(R_{y1} ), C(O), C(O)O, OC(O), C(O)N(R_{y1} ), or N(R_{y1} )C(O), wherein R_{y1} is selected from hydrogen or (1-6 C)alkyl;
   L₂ is absent or (1-3 C)alkylene; and
   Q₁ is hydrogen, (1-6 C)alkyl, phenyl, (3-8 C)cycloalkyl, 5 or 6 -membered heteroaryl or 4 to 7 -membered heterocyclyl;
   wherein Q is optionally further substituted by one or more substituent groups independently selected from oxo, (1-6 C)alkyl, halo, (1-4 C)haloalkyl, (1 -4 C)haloalkoxy, (1-4 C)aminoalkyl, (1-4 C)hydroxyalkyl, cyano, or by one or more group(s) of the formula:

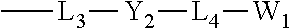

wherein:
   L₃ is absent or (1-4 C)alkylene;
   Y₂ is absent or selected from or O, S, SO, SO₂, N(R_{y2} ), C(O), C(O)O, OC(O), C(O)N(R_{y2} ), N(R_{y2} )C(O) or S(O)₂ N(R_{y2} ), N(R_{y2} )SO₂ wherein R_{y2} is selected from hydrogen or (1-4 C)alkyl;
   L₄ is absent or (1-3 C)alkylene; and
   W₁ is hydrogen, (1-6 C)alkyl, aryl, (3-8 C)cycloalkyl, 5 or 6 -membered heteroaryl or 4 to 7 -membered heterocyclyl;
   wherein W₁ is optionally substituted by one or more substituents selected from oxo, (1-4 C)alkyl, halo, (1-4 C)haloalkyl, (1-4 C)haloalkoxy, (1 -4 C)alkoxy, amino, (1-4 C)alkylamino, di[(1-4 C)alkyl]amino C(O)OH, C(O)O(1-4 C)alkyl, (CH₂ )₀₋3 -[4 to 7 -membered heterocyclyl] or cyano.
(19 ) Q is hydrogen, halo, cyano or a group of the formula:

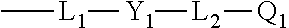

wherein:
   L₁ is absent or (1-4 C)alkylene;
   Y₁ is absent or O, S, SO, SO₂, N(R_{y1} ), C(O), C(O)O, OC(O), C(O)N(R_{y1} ), or N(R_{y1} )C(O), wherein R_{y1} is selected from hydrogen or (1-4 C)alkyl;
   L₂ is absent or (1-3 C)alkylene; and
   Q₁ is hydrogen, (1-6 C)alkyl, phenyl, (3-8 C)cycloalkyl, 5 or 6 -membered heteroaryl or 4 to 7 -membered heterocyclyl;
   wherein Q is optionally further substituted by one or more substituent groups independently selected from oxo, (1-6 C)alkyl, halo, (1-4 C)haloalkyl, (1 -4 C)haloalkoxy, (1-4 C)aminoalkyl, (1-4 C)hydroxyalkyl, cyano, or by one or more group(s) of the formula:

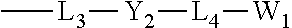

wherein:
   L₃ is absent or (1-3 C)alkylene;
   Y₂ is absent or selected from or O, S, SO, SO₂, N(R_{y2} ), C(O), C(O)O, OC(O), C(O)N(R_{y2} ), N(R_{y2} )C(O), S(O)₂ N(R_{y2} ) or N(R_{y2} )SO₂ wherein R_{y2} is selected from hydrogen or (1-2 C)alkyl;
   L₄ is absent or (1-3 C)alkylene; and
   W₁ is hydrogen, (1-6 C)alkyl, aryl, (3-8 C)cycloalkyl, 5 or 6 -membered heteroaryl or 4 to 7 -membered heterocyclyl;
   wherein W₁ is optionally substituted by one or more substituents selected from oxo, (1-4 C)alkyl, halo, (1-4 C)haloalkyl, (1-4 C)haloalkoxy, (1 -4 C)alkoxy, amino, (1-4 C)alkylamino, di[(1-4 C)alkyl]amino C(O)OH, C(O)O(1-4 C)alkyl, (CH₂ )₀₋₃ -[4 to 7 -membered heterocyclyl] or cyano;
(20 ) Q is a group of the formula:

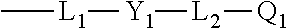

wherein:
L₁ is absent or (1-3 C)alkylene;
Y₁ is absent or O, S, N(R_{y1} ), C(O), C(O)O, C(O)N(R_{y1} ), or N(R_{y1} )C(O), wherein
R_{y1} is selected from hydrogen or (1-4 C)alkyl;
L₂ is absent or methylene; and
Q₁ is hydrogen, (1-6 C)alkyl, phenyl, (3-8 C)cycloalkyl, 5 or 6 -membered heteroaryl or 4 to 7 -membered heterocyclyl;
wherein Q is optionally further substituted by one or more substituent groups independently selected from oxo, (1-6 C)alkyl, halo, (1-2 C)haloalkyl, (1 -2 C)haloalkoxy, cyano, or by one or more group(s) of the formula:

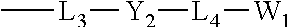

wherein:
   L₃ is absent or (1-4 C)alkylene;
   Y₂ is absent or selected from or O, SO₂, N(R_{y2} ), C(O), C(O)O, C(O)N(R_{y2} ) or N(R_{y2} )SO₂ wherein R_{y2} is selected from hydrogen or (1-2 C)alkyl;
   L₄ is absent or (1-3 C)alkylene; and
   W₁ is hydrogen, (1-6 C)alkyl, aryl, (3-8 C)cycloalkyl, 5 or 6 -membered heteroaryl or 4 to 7 -membered heterocyclyl;
wherein W₁ is optionally substituted by one or more substituents selected from oxo, (1 -4 C)alkyl, halo, (1-2 C)haloalkyl, (1-2 C)haloalkoxy, (1-4 C)alkoxy, C(O)OH, C(O)O(1 -4 C)alkyl, (CH₂ )₀₋₃ -[4 to 7 -membered heterocyclyl] or cyano.
(21 ) Q is a group selected from hydrogen, halo, cyano, (1-6 C)alkyl, (1-6 C)alkoxy, (1 -6 C)haloalkyl, (1-6 C)haloalkoxy, or a group of with a formula selected from:

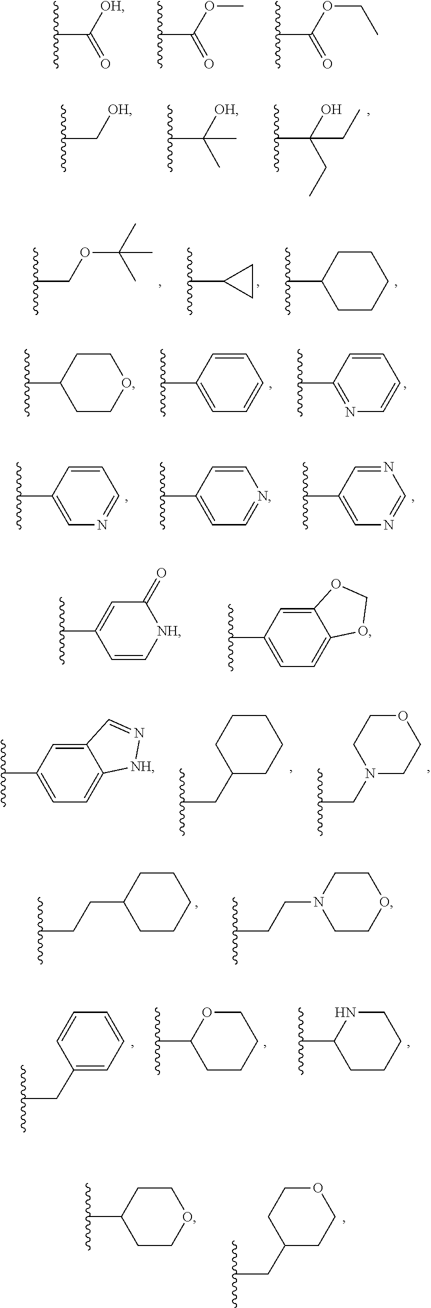

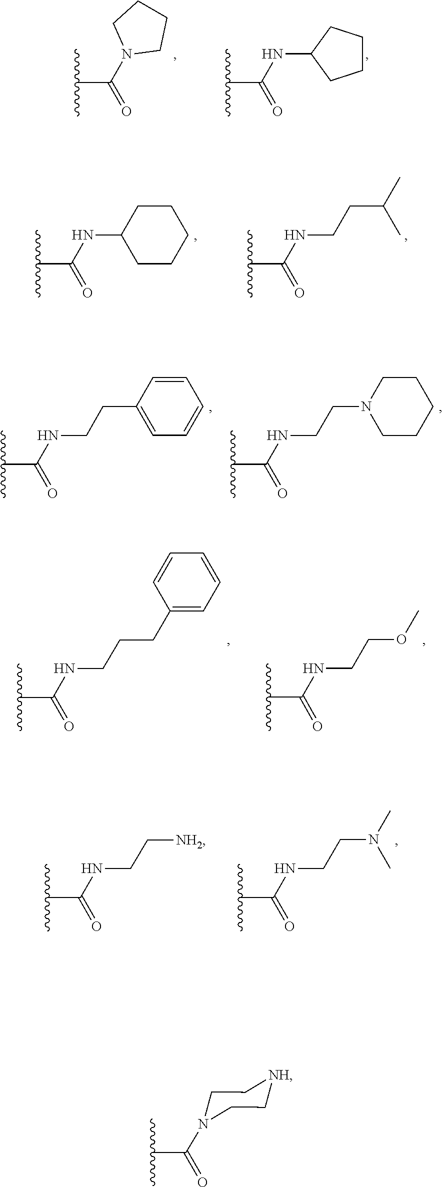

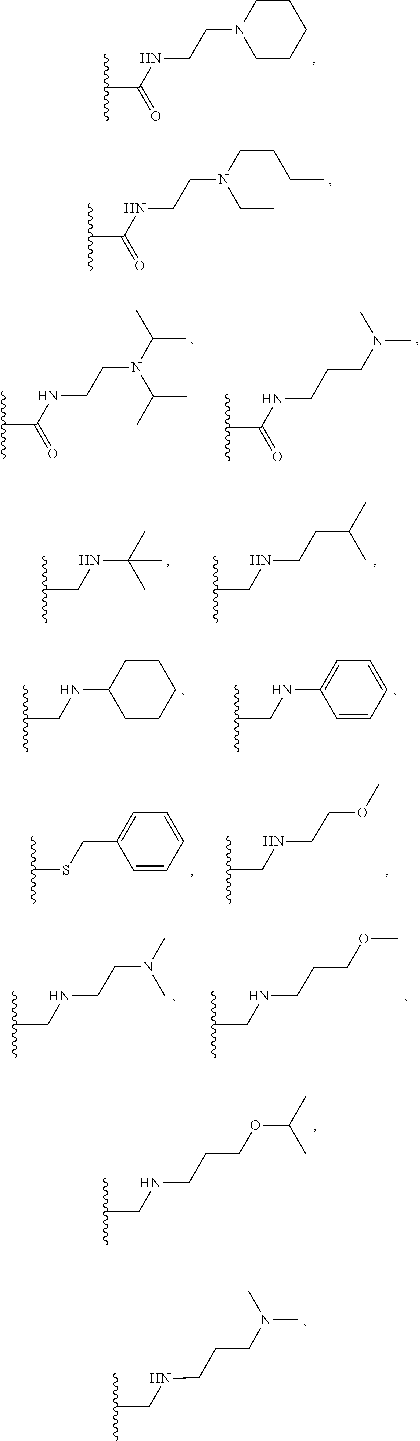

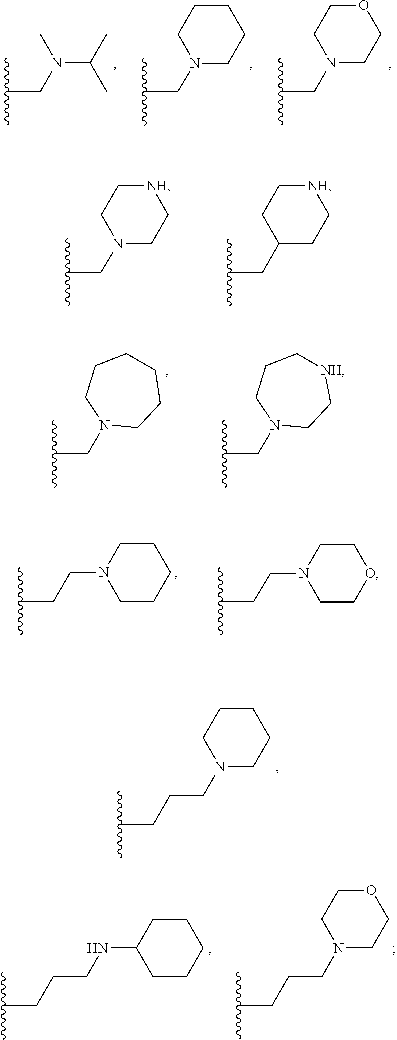

wherein any cycloalkyl, heterocyclyl, aryl or heteroaryl ring above may be optionally substituted by one or more substituents selected from oxo, (1-6 C)alkyl, halo, (1-2 C)haloalkyl, (1 -2 C)haloalkoxy, cyano, or by one or more group(s) of the formula:

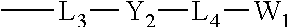

wherein L₃, Y₂, L₄ and W₁ are as defined herein.
(22 ) R₄ is hydrogen or fluoro.
(23 ) R₄ is hydrogen.
(24 ) R₄ is fluoro.
(25 ) X₁ is N or CR₅, wherein R₅ is selected from hydrogen, halo, or cyano.
(26 ) x is independently selected from 0, 1 or 2 ;
(27 ) x is 0 ;
(28 ) x is 1 ;
(29 ) x is 2 ;
(30 ) y1 is independently selected from 0, 1 or 2 ;
(31 ) y1 is 0 ;
(32 ) y1 is 1 ;
(33 ) y1 is 2 ;
(34 ) y2 is independently selected from 0, 1 or 2 ;
(35 ) y2 is 0 ;
(36 ) y2 is 1 ;
(37 ) y2 is 2 ;
(38 ) y3 is independently selected from 0, 1 or 2 ;
(39 ) y3 is 0 ;
(40 ) y3 is 1 ;
(41 ) y3 is 2 ;
(42 ) y4 is independently selected from 0, 1 or 2 ;
(43 ) y4 is 0 ;
(44 ) y4 is 1 ;
(45 ) y4 is 2 ;
(46 ) z is independently selected from 0, 1 or 2 ;
(47 ) z is 0 ;
(48 ) z is 1 ;
(49 ) z is 2;

Suitably, R₁ is as defined in any one of numbered paragraphs (1 ) to (8 ) above. More suitably, R₁ is as defined in any one of numbered paragraphs (5 ) to (8 ) above. Most suitably, R₁ is as defined in numbered paragraph (7 ) or (8 ) above.

Suitably, either:
a) R₂ hydrogen and R₃ is as defined in any one of numbered paragraphs (9 ) to (13 ) above, or
b) R₂ and R₃ are linked such that together they form a —X₂ ═CQ- group, X₂ is as defined in any one of paragraphs (15 ) to (17 ) above and Q is as defined in any one of numbered paragraphs (18 ) to (21 ) above.

More suitably, either:
a) R₂ hydrogen and R₃ is as defined in any one of numbered paragraphs (11 ) to (13 ) above, or
b) R₂ and R₃ are linked such that together they form a —X₂ ═CQ- group, X₂ is as defined paragraph (16 ) or (17 ) above and Q is as defined in numbered paragraph (19 ), (20 ) or (21 ) above.

Most suitably, either:
a) R₂ hydrogen and R₃ is as defined in numbered paragraph (13 ) above, or
b) R₂ and R₃ are linked such that together they form a —X₂ ═CQ- group, X₂ is as defined in numbered paragraph (17 ) above and Q is as defined in paragraph (20 ) or (21 ) above.

Suitably, R₄ is as defined in any one of numbered paragraphs (22 ) to (24 ) above. More suitably, R₄ is as defined in numbered paragraph (23 ) or (24 ) above.

Suitably, Q is as defined in any one of numbered paragraphs (18 ) to (21 ) above. More suitably, Q is as defined in numbered paragraph (19 ), (20 ) or (21 ) above. Most suitably, Q is as defined in numbered paragraph (20 ) or (21 ) above.

Suitably, X₁ is as defined in paragraph (24 ).

Suitably, X₂ is as defined in any one of numbered paragraphs (15 ) to (17 ) above. More suitably, X₂ is as defined in numbered paragraph (16 ) or (17 ) above. Most suitably, X₂ is as defined in numbered paragraph (17 ) above.

Suitably, x is as defined in any one of numbered paragraphs (26 ) to (29 ) above. More suitably, x is as defined in numbered paragraph (26 ) above.

Suitably, y1 is as defined in any one of numbered paragraphs (30 ) to (33 ) above. More suitably, y1 is as defined in numbered paragraph (30 ) above.

Suitably, y2 is as defined in any one of numbered paragraphs (34 ) to (37 ) above. More suitably, y2 is as defined in numbered paragraph (34 ) above.

Suitably, y3 is as defined in any one of numbered paragraphs (38 ) to (41 ) above. More suitably, y3 is as defined in numbered paragraph (38 ) above.

Suitably, y4 is as defined in any one of numbered paragraphs (42 ) to (45 ) above. More suitably, y4 is as defined in numbered paragraph (42 ) above.

Suitably, z is as defined in any one of numbered paragraphs (46 ) to (49 ) above. More suitably, z is as defined in numbered paragraph (46 ) above.

As indicated above, particular compounds of the invention include, for example, compounds of the Formula (I), or pharmaceutically acceptable salts, hydrates and/or solvates thereof, defined herein having one of the structural formulae (Ia), (Ib) or (Ic) shown below:

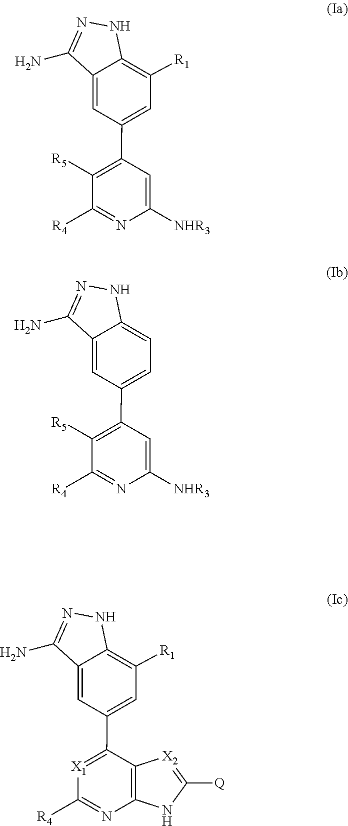

wherein R₁, X₁, X₂, R₃, R₄, R₅ and Q are each as defined herein.

In a particular group of compounds of the invention, compounds have a structure according to formula Ia (which is a sub-definition of formula I), or a pharmaceutically acceptable salt, hydrate and/or solvate thereof, wherein R₁, X₁, X₂, R₃, R₄, R₅ and Q each have any one of the definitions set out herein.

In an embodiment of the compounds of formula Ia, or a pharmaceutically acceptable salt, hydrate and/or solvate thereof:
R₁ is as defined in any one of numbered paragraphs (1 ) to (8 ) above;
R₃ is as defined in any one of numbered paragraphs (9 ) to (13 ) above;
R₄ is as defined in any one of numbered paragraphs (22 ) to (24 ) above; and
R₅ is as defined in numbered paragraph (25 ) above.

In an embodiment of the compounds of formula Ia, or a pharmaceutically acceptable salt, hydrate and/or solvate thereof:
Rₗ is as defined in any one of numbered paragraphs (5 ) to (8 ) above;
R₃ is as defined in any one of numbered paragraphs (11 ) to (13 ) above;
R₄ is as defined numbered paragraph (23 ) or (24 ) above; and
R₅ is as defined in numbered paragraph (25 ) above.

In an embodiment of the compounds of formula Ia, or a pharmaceutically acceptable salt, hydrate and/or solvate thereof:
Rₗ is as defined in numbered paragraph (7 ) or (8 ) above;
R₃ is as defined in paragraph (13 ) above;
R₄ is as defined in numbered paragraph (23 ) or (24 ) above; and
R₅ is as defined in numbered paragraph (25 ) above.

In a particular group of compounds of the invention, compounds have a structure according to formula Ib (which is a sub-definition of formula I), or a pharmaceutically acceptable salt, hydrate and/or solvate thereof, wherein R₃, R₄, and R₅ each have any one of the definitions set out herein.

In an embodiment of the compounds of formula Ib, or a pharmaceutically acceptable salt, hydrate and/or solvate thereof:
R₃ is as defined in any one of numbered paragraphs (9 ) to (13 ) above;
R₄ is as defined in any one of numbered paragraphs (22 ) to (24 ) above; and
R₅ is as defined in numbered paragraph (25 ) above.

In an embodiment of the compounds of formula Ib, or a pharmaceutically acceptable salt, hydrate and/or solvate thereof:
R₃ is as defined in any one of numbered paragraphs (11 ) to (13 ) above;
R₄ is as defined numbered paragraph (23 ) or (24 ) above; and
R₅ is as defined in numbered paragraph (25 ) above.

In an embodiment of the compounds of formula Ib, or a pharmaceutically acceptable salt, hydrate and/or solvate thereof:
R₃ is as defined in paragraph (13 ) above;
R₄ is as defined in numbered paragraph (23 ) or (24 ) above; and
R₅ is as defined in numbered paragraph (25 ) above.

In a particular group of compounds of the invention, compounds have a structure according to formula Ic (which is a sub-definition of formula I), or a pharmaceutically acceptable salt, hydrate and/or solvate thereof, wherein R₁, R₄, X₁, X₂ and Q each have any one of the definitions set out herein.

In an embodiment of the compounds of formula Ic, or a pharmaceutically acceptable salt, hydrate and/or solvate thereof:
R₁ is as defined in any one of numbered paragraphs (1 ) to (8 ) above;
R₄ is as defined in any one of numbered paragraphs (22 ) to (24 ) above;
X₁ is as defined in numbered paragraph (25 ) above;
X₂ is as defined in any one of numbered paragraphs (15 ) to (17 ) above; and
Q is as defined in any one of numbered paragraphs (18 ) to (21 ) above.

In an embodiment of the compounds of formula Ic, or a pharmaceutically acceptable salt, hydrate and/or solvate thereof:
Rₗ is as defined in any one of numbered paragraphs (5 ) to (8 ) above;
R₄ is as defined numbered paragraph (23 ) or (24 ) above;
X₁ is as defined in numbered paragraph (25 ) above;
X₂ is as defined in numbered paragraph (16 ) or (17 ) above; and
Q is as defined in numbered paragraph (19 ), (20 ) or (21 ) above.

In an embodiment of the compounds of formula Ic, or a pharmaceutically acceptable salt, hydrate and/or solvate thereof:
Rₗ is as defined in numbered paragraph (7 ) or (8 ) above;
R₄ is as defined in numbered paragraph (23 ) or (24 ) above;
X₁ is as defined in numbered paragraph (25 ) above;
X₂ is as defined in numbered paragraph (17 ) above; and
Q is as defined in numbered paragraph (20 ) or (21 ) above.

Particular compounds of the present invention include any of the compounds exemplified in the present application, or a pharmaceutically acceptable salt or solvate thereof, and, in particular, any of the following:
5-(2 -aminopyridin-4 -yl)-7 -chloro-1 H-indazol-3 -amine
5-(2 -aminopyridin-4 -yl)-7 -methyl-1 H-indazol-3 -amine
5-(2 -aminopyridin-4 -yl)-7-(trifluoromethyl)-1 H-indazol-3 -amine
5-(2-(ethylamino)pyridin-4 -yl)-1 H-indazol-3 -amine
5-(2-(propylamino)pyridin-4 -yl)-1 H-indazol-3 -amine
5-(2-(isopropylamino)pyridin-4 -yl)-1 H-indazol-3 -amine
5-(2-((cyclopropylmethyl)amino)pyridin-4 -yl)-1 H-indazol-3 -amine
5-(2-(isopentylamino)pyridin-4 -yl)-1 H-indazol-3 -amine
5-(2-(hexylamino)pyridin-4 -yl)-1 H-indazol-3 -amine
5-(2-(cyclohexylamino)pyridin-4 -yl)-1 H-indazol-3 -amine
5 -{2 -[(Trans-4 -methylcyclohexyl)amino]pyridin-4 -y1 }-1 H-indazol-3 -amine
2-((4-(3 -amino-1 H-indazol-5 -yl)pyridin-2 -yl)amino)ethan-1 -ol
3-((4-(3 -amino-1 H-indazol-5 -yl)pyridin-2 -yl)amino)propan-1 -ol
4-((4-(3 -amino-1 H-indazol-5 -yl)pyridin-2 -yl)amino)butan-1 -ol
5-((4-(3 -amino-1 H-indazol-5 -yl)pyridin-2 -yl)amino)pentan-1 -ol
5 -{2 -[(trans-4 -hydroxycyclohexyl)amino]pyridin-4 -y1 }-1 H-indazol-3 -amine I
5-(2-((2 -methoxyethyl)amino)pyridin-4 -yl)-1 H-indazol-3 -amine
5-(2-((3 -methoxypropyl)amino)pyridin-4 -yl)-1 H-indazol-3 -amine
5-(2-((3 -isopropoxypropyl)amino)pyridin-4 -yl)-1 H-indazol-3 -amine
3-((4-(3 -amino-1 H-indazol-5 -yl)pyrimidin-2 -yl)amino)propan-1 -ol
3-((4-(3 -amino-1 H-indazol-5 -yl)pyridin-2 -yl)(methyl)amino)propan-1 -ol
5-(2-((2 -morpholinoethyl)amino)pyridin-4 -yl)-1 H-indazol-3 -amine
5-(2-((2-(piperidin-1 -yl)ethyl)amino)pyridin-4 -yl)-1 H-indazol-3 -amine
N¹-(4-(3 -amino-1 H-indazol-5 -yl)pyridin-2 -yl)-N3 -methylpropane-1,3 -diamine
5-(2-(benzylamino)pyridin-4 -yl)-1 H-indazol-3 -amine
3-(((4-(3 -amino-1 H-indazol-5 -yl)pyridin-2 -yl)amino)methyl)benzonitrile
5-(2-((3 -methoxybenzyl)amino)pyridin-4 -yl)-1 H-indazol-3 -amine
2-(3-(((4-(3 -amino-1 H-indazol-5 -yl)pyridin-2 -yl)amino)methyl)phenyl)propan-2 -ol
5-(2-((4-(trifluoromethyl)benzyl)amino)pyridin-4 -yl)-1 H-indazol-3 -amine
4-(((4-(3 -amino-1 H-indazol-5 -yl)pyridin-2 -yl)amino)methyl)benzonitrile
5-(2-((4-(tert-butyl)benzyl)amino)pyridin-4 -yl)-1 H-indazol-3 -amine
2-(4-(((4-(3 -amino-1 H-indazol-5 -yl)pyridin-2 -yl)amino)methyl)phenyl)propan-2 -ol
5-(2-((4-(methylsulfonyl)benzyl)amino)pyridin-4 -yl)-1 H-indazol-3 -amine
5-(2-((furan-3 -ylmethyl)amino)pyridin-4 -yl)-1 H-indazol-3 -amine
5-(2-((pyridin-2 -ylmethyl)amino)pyridin-4 -yl)-1 H-indazol-3 -amine
5-(2-(phenethylamino)pyridin-4 -yl)-1 H-indazol-3 -amine
5-(2-((2-(pyridin-2 -yl)ethyl)amino)pyridin-4 -yl)-1 H-indazol-3 -amine
5-(2-((2-(pyridin-3 -yl)ethyl)amino)pyridin-4 -yl)-1 H-indazol-3 -amine
5-(2-((2-(pyridin-4 -yl)ethyl)amino)pyridin-4 -yl)-1 H-indazol-3 -amine
5-(2-((2-(1 H-indol-3 -yl)ethyl)amino)pyridin-4 -yl)-1 H-indazol-3 -amine
5-(2-((4 -fluorophenethyl)amino)pyridin-4 -yl)-1 H-indazol-3 -amine
5-(2-((4 -chlorophenethyl)amino)pyridin-4 -yl)-1 H-indazol-3 -amine
4-(2-((4-(3 -amino-1 H-indazol-5 -yl)pyridin-2 -yl)amino)ethyl)phenol
5-(2-((4 -methoxyphenethyl)amino)pyridin-4 -yl)-1 H-indazol-3 -amine
5-(2-((4-(tert-butyl)phenethyl)amino)pyridin-4 -yl)-1 H-indazol-3 -amine
4-(2-((4-(3 -amino-1 H-indazol-5 -yl)pyridin-2 -yl)amino)ethyl)benzenesulfonamide
5-(2-((3 -chlorophenethyl)amino)pyridin-4 -yl)-1 H-indazol-3 -amine
5-(2-((2-(trifluoromethyl)phenethyl)amino)pyridin-4 -yl)-1 H-indazol-3 -amine
5-(2-((3 -phenylpropyl)amino)pyridin-4 -yl)-1 H-indazol-3 -amine
5-(2-((2 -phenoxyethyl)amino)pyridin-4 -yl)-1 H-indazol-3 -amine
N-(4-(3 -amino-1 H-indazol-5 -yl)pyridin-2 -yl)cyclopropanecarboxamide
N-(4-(3 -amino-1 H-indazol-5 -yl)pyridin-2 -yl)benzamide
N-(4-(3 -amino-1 H-indazol-5 -yl)pyridin-2 -yl)-2 -phenylacetamide
N-(4-(3 -amino-1 H-indazol-5 -yl)pyridin-2 -yl)-2-(3 -fluorophenyl)acetamide
N-(4-(3 -amino-1 H-indazol-5 -yl)pyridin-2 -yl)-2-(3 -tolyl)acetamide
N-(4-(3 -amino-1 H-indazol-5 -yl)pyridin-2 -yl)-2-(3-(trifluoromethyl)phenyl)acetamide
N-(4-(3 -amino-1 H-indazol-5 -yl)pyridin-2 -yl)-2-(3 -chlorophenyl)acetamide
N-(4-(3 -amino-1 H-indazol-5 -yl)pyridin-2 -yl)-2-(3 -aminophenyl)acetamide
N-(4-(3 -amino-1 H-indazol-5 -yl)pyridin-2 -yl)-2-(3 -nitrophenyl)acetamide
N-(4-(3 -amino-1 H-indazol-5 -yl)pyridin-2 -yl)-2-(3-(methylsulfonyl)phenyl)acetamide
N-(4-(3 -amino-1 H-indazol-5 -yl)pyridin-2 -yl)-2-(3 -methoxyphenyl)acetamide
N-(4-(3 -amino-1 H-indazol-5 -yl)pyridin-2 -yl)-2-(3-(benzyloxy)phenyl)acetamide tert-butyl (3-(2-((4-(3 -amino-1 H-indazol-5 -yl)pyridin-2 -yl)amino)-2 -oxoethyl)phenyl)carbamate
N-(4-(3 -amino-1 H-indazol-5 -yl)pyridin-2 -yl)-2-(4 -fluorophenyl)acetamide
N-(4-(3 -amino-1 H-indazol-5 -yl)pyridin-2 -yl)-2-(p-tolyl)acetamide
N-(4-(3 -amino-1 H-indazol-5 -yl)pyridin-2 -yl)-2-(4-(trifluoromethyl)phenyl)acetamide
N-(4-(3 -amino-1 H-indazol-5 -yl)pyridin-2 -yl)-2-(4 -chlorophenyl)acetamide
N-(4-(3 -amino-1 H-indazol-5 -yl)pyridin-2 -yl)-2-(4-(methylthio)phenyl)acetamide
N-(4-(3 -amino-1 H-indazol-5 -yl)pyridin-2 -yl)-2-(4-(methylsulfonyl)phenyl)acetamide
N-(4-(3 -amino-1 H-indazol-5 -yl)pyridin-2 -yl)-2-(4 -methoxyphenyl)acetamide
N-(4-(3 -amino-1 H-indazol-5 -yl)pyridin-2 -yl)-2-(4 -aminophenyl)acetamide tert-butyl (4-(2-((4-(3 -amino-1 H-indazol-5 -yl)pyridin-2 -yl)amino)-2 -oxoethyl)phenyl)carbamate
N-(4-(3 -amino-1 H-indazol-5 -yl)pyridin-2 -yl)-2-(2 -fluorophenyl)acetamide
N-(4-(3 -amino-1 H-indazol-5 -yl)pyridin-2 -yl)-2-(o-tolyl)acetamide
N-(4-(3 -amino-1 H-indazol-5 -yl)pyridin-2 -yl)-2-(2-(trifluoromethyl)phenyl)acetamide
N-(4-(3 -amino-1 H-indazol-5 -yl)pyridin-2 -yl)-2-(2 -chlorophenyl)acetamide
N-(4-(3 -amino-1 H-indazol-5 -yl)pyridin-2 -yl)-2-(2 -methoxyphenyl)acetamide
N-(4-(3 -amino-1 H-indazol-5 -yl)pyridin-2 -yl)-2-(pyridin-2 -yl)acetamide
N-(4-(3 -amino-1 H-indazol-5 -yl)pyridin-2 -yl)-2-(pyridin-3 -yl)acetamide
N-(4-(3 -amino-1 H-indazol-5 -yl)pyridine-2 -yl)-2-(pyridine-4 -yl)acetamide
N-(4-(3 -amino-1 H-indazol-5 -yl)pyridine-2 -yl)-3 -phenylpropanamide
ethyl (4-(3 -amino-1 H-indazol-5 -yl)pyridine-2 -yl)carbamate
1-(4-(3 -amino-1 H-indazol-5 -yl)pyridine-2 -yl)-3 -ethylurea
1-(4-(3 -amino-1 H-indazol-5 -yl)pyridine-2 -yl)-3 -propylurea
1-(4-(3 -amino-1 H-indazol-5 -yl)pyridine-2 -yl)-3 -isopentylurea
1-(4-(3 -amino-1 H-indazol-5 -yl)pyridine-2 -yl)-3 -cyclopentylurea
1-(4-(3 -amino-1 H-indazol-5 -yl)pyridine-2 -yl)-3 -cyclohexylurea
1-(4-(3 -amino-1 H-indazol-5 -yl)pyridine-2 -yl)-3-(2 -hydroxyethyl)urea
1-(4-(3 -amino-1 H-indazol-5 -yl)pyridine-2 -yl)-3-(3 -hydroxypropyl)urea
1-(4-(3 -amino-1 H-indazol-5 -yl)pyridine-2 -yl)-3-(2 -methoxyethyl)urea
3-(4-(3 -amino-1 H-indazol-5 -yl)pyridine-2 -yl)-1-(2 -hydroxyethyl)-1 -methylurea
1-(4-(3 -amino-1 H-indazol-5 -yl)pyridine-2 -yl)-3 -benzylurea
1-(4-(3 -amino-1 H-indazol-5 -yl)pyridine-2 -yl)-3 -phenethylurea
1-(4-(3 -amino-1 H-indazol-5 -yl)pyridine-2 -yl)-3-(pyridine-2 -ylmethyl)urea
1-(4-(3 -amino-1 H-indazol-5 -yl)pyridine-2 -yl)-3-(pyridine-3 -ylmethyl)urea
1-(4-(3 -amino-1 H-indazol-5 -yl)pyridine-2 -yl)-3-(pyridine-4 -ylmethyl)urea
1-(4-(3 -amino-1 H-indazol-5 -yl)pyridine-2 -yl)-3 -phenylurea
1-(4-(3 -amino-1 H-indazol-5 -yl)pyridine-2 -yl)-3-(3 -fluorophenyl)urea
1-(4-(3 -amino-1 H-indazol-5 -yl)pyridine-2 -yl)-3-(3 -chlorophenyl)urea
1-(4-(3 -amino-1 H-indazol-5 -yl)pyridine-2 -yl)-3-(3 -isopropylphenyl)urea
1-(4-(3 -amino-1 H-indazol-5 -yl)pyridine-2 -yl)-3-(3-(hydroxymethyl)phenyl)urea
3-(3-(4-(3 -amino-1 H-indazol-5 -yl)pyridine-2 -yl)ureido)benzamide
1-(4-(3 -amino-1 H-indazol-5 -yl)pyridine-2 -yl)-3-(3 -phenoxyphenyl)urea
1-(4-(3 -amino-1 H-indazol-5 -yl)pyridine-2 -yl)-3-(3-(benzyloxy)phenyl)urea
1-(4-(3 -amino-1 H-indazol-5 -yl)pyridine-2 -yl)-3-(3-((4 -fluorobenzyl)oxy)phenyl)urea
1-(4-(3 -amino-1 H-indazol-5 -yl)pyridine-2 -yl)-3-(3-((3 -fluorobenzyl)oxy)phenyl)urea
1-(4-(3 -amino-1 H-indazol-5 -yl)pyridine-2 -yl)-3-(3-((2 -fluorobenzyl)oxy)phenyl)urea
3-(3-(4-(3 -amino-1 H-indazol-5 -yl)pyridine-2 -yl)ureido)-N-phenylbenzamide
1-(4-(3 -amino-1 H-indazol-5 -yl)pyridine-2 -yl)-3-(4 -fluorophenyl)urea
1-(4-(3 -amino-1 H-indazol-5 -yl)pyridine-2 -yl)-3 -(4 -chlorophenyl)urea
1 -(4 -(3 -amino-1 H-indazol-5 -yl)pyridine-2 -yl)-3 -(4 -(tert-butyl)phenyl)urea
1 -(4 -(3 -amino-1 H-indazol-5 -yl)pyridine-2 -yl)-3 -(4 -(methylsulfonyl)phenyl)urea
1 -(4 -(3 -amino-1 H-indazol-5 -yl)pyridin-2 -yl)-3 -(o-tolyl)urea
1 -(4 -(3 -amino-1 H-indazol-5 -yl)pyridine-2 -yl)-3 -(2 -ethylphenyl)urea
1 -(4 -(3 -amino-1 H-indazol-5 -yl)pyridine-2 -yl)-3 -(2 -isopropylphenyl)urea
1 -(4 -(3 -amino-1 H-indazol-5 -yl)pyridine-2 -yl)-3 -(pyridine-3 -yl)urea
5 -(2 -(phenylamino)pyridine-4 -yl)-1 H-indazol-3 -amine
5 -(2 -((3 -isopropylphenyl)amino)pyridine-4 -yl)-1 H-indazol-3 -amine
3 -((4 -(3 -amino-1 H-indazol-5 -yl)pyridine-2 -yl)amino)phenol
(3 -((4 -(3 -amino-1 H-indazol-5 -yl)pyridine-2 -yl)amino)phenyl)methanol
3 -((4 -(3 -amino-1 H-indazol-5 -yl)pyridine-2 -yl)amino)benzoic acid
ethyl 3 -((4 -(3 -amino-1 H-indazol-5 -yl)pyridine-2 -yl)amino)benzoate
3 -((4 -(3 -amino-1 H-indazol-5 -yl)pyridine-2 -yl)amino)benzamide
3 -((4 -(3 -amino-1 H-indazol-5 -yl)pyridine-2 -yl)amino)-N-(2 -hydroxyethyl)benzamide
N1 -(4 -(3 -amino-1 H-indazol-5 -yl)pyridine-2 -yl)benzene-1,3 -diamine
N-(3 -((4 -(3 -amino-1 H-indazol-5 -yl)pyridin-2 -yl)amino)phenyl)acetamide
N-(3 -((4 -(3 -amino-1 H-indazol-5 -yl)pyridine-2 -yl)amino)phenyl)benzamide
5 -(2 -((3 -phenoxyphenyl)amino)pyridine-4 -yl)-1 H-indazol-3 -amine
5 -(2 -((3 -(benzyloxy)phenyl)amino)pyridine-4 -yl)-1 H-indazol-3 -amine
5 -(2 -((4 -fluorophenyl)amino)pyridine-4 -yl)-1 H-indazol-3 -amine
5 -(2 -((4 -chlorophenyl)amino)pyridine-4 -yl)-1 H-indazol-3 -amine
4 -((4 -(3 -amino-1 H-indazol-5 -yl)pyridine-2 -yl)amino)phenol
5 -(2 -((4 -methoxyphenyl)amino)pyridine-4 -yl)-1 H-indazol-3 -amine
5 -(2 -((4 -(trifluoromethoxy)phenyl)amino)pyridine-4 -yl)-1 H-indazol-3 -amine
5 -(2 -((4 -propoxyphenyl)amino)pyridine-4 -yl)-1 H-indazol-3 -amine
2 -((4 -(3 -amino-1 H-indazol-5 -yl)pyridine-2 -yl)amino)phenol
4 -((4 -(3 -amino-1 H-indazol-5 -yl)pyridine-2 -yl)amino)-2 -methylphenol
5 -((4 -(3 -amino-1 H-indazol-5 -yl)pyridine-2 -yl)amino)-2 -methylphenol
4 -((4 -(3 -amino-1 H-indazol-5 -yl)pyridine-2 -yl)amino)-3 -methylphenol
5 -(2 -((3,4 -dichlorophenyl)amino)pyridine-4 -yl)-1 H-indazol-3 -amine
N-(4 -(3 -amino-1 H-indazol-5 -yl)pyridin-2 -yl)thiazol-2 -amine
5 -(2 -(pyrimidin-2 -ylamino)pyridine-4 -yl)-1 H-indazol-3 -amine
5 -(2 -(pyridine-4 -ylamino)pyridine-4 -yl)-1 H-indazol-3 -amine
5 -(2 -(pyridine-3 -ylamino)pyridine-4 -yl)-1 H-indazol-3 -amine
5 -(2 -(pyridine-2 -ylamino)pyridine-4 -yl)-1 H-indazol-3 -amine
N2 -(4 -(3 -amino-1 H-indazol-5 -yl)pyridine-2 -yl)pyridine-2,6 -diamine
N2 -(4 -(3 -amino-1 H-indazol-5 -yl)pyridine-2 -yl)-N6 -benzylpyridine-2,6 -diamine
5 -(7 H-pyrrolo[2,3 -b]pyrimidin-4 -yl)-1 H-indazol-3 -amine
2 -amino-4 -(3 -amino-1 H-indazol-5 -yl)-7 H-pyrrolo[2,3 -b]pyrimidine-5 -carbonitrile
5 -(3 H-imidazo[4,5 -b]pyridin-7 -yl)-1 H-indazol-3 -amine
5 -(3 -methyl-1 H-pyrrolo[2,3 -b]pyridin-4 -yl)-1 H-indazol-3 -amine
5 -(3 -chloro-1 H-pyrrolo[2,3 -b]pyridin-4 -yl)-1 H-indazol-3 -amine
4 -(3 -amino-1 H-indazol-5 -yl)-1 H-pyrrolo[2,3 -b]pyridine-3 -carbonitrile
5 -(2 -methyl-1 H-pyrrolo[2,3 -b]pyridin-4 -yl)-1 H-indazol-3 -amine
5 -(2 -(tert-butyl)-1 H-pyrrolo[2,3 -b]pyridin-4 -yl)-1 H-indazol-3 -amine
5 -(2 -phenyl-1 H-pyrrolo[2,3 -b]pyridin-4 -yl)-1 H-indazol-3 -amine
4 -(3 -amino-1 H-indazol-5 -yl)-1 H-pyrrolo[2,3 -b]pyridine-2 -carboxylic acid
5 -(5 -fluoro-1 H-pyrrolo[2,3 -b]pyridin-4 -yl)-1 H-indazol-3 -amine
5 -(5 -chloro-1 H-pyrrolo[2,3 -b]pyridin-4 -yl)-1 H-indazol-3 -amine
4 -(3 -amino-1 H-indazol-5 -yl)-1 H-pyrrolo[2,3 -b]pyridine-5 -carbonitrile
5 -(7 H-pyrrolo[2,3 -b]pyrimidin-4 -yl)-1 H-indazol-3 -amine
7 -bromo-5 -(7 H-pyrrolo[2,3 -b][2,3 -b]pyrimidin-4 -yl)-1 H-indazol-3 -amine
5 -(2 -methyl-1 H-pyrrolo[2,3 -b]pyridin-4 -yl)-1 H-indazol-3 -amine
5 -(2 -(tert-butyl)-1 H-pyrrolo[2,3 -b]pyridin-4 -yl)-1 H-indazol-3 -amine
5 -(2 -cyclopropyl-1 H-pyrrolo[2,3 -b]pyridin-4 -yl)-1 H-indazol-3 -amine
5 -(2 -cyclohexyl-1 H-pyrrolo[2,3 -b]pyridin-4 -yl)-1 H-indazol-3 -amine
5 -(2 -neopentyl-1 H-pyrrolo[2,3 -b]pyridin-4 -yl)-1 H-indazol-3 -amine
5 -(2 -(cyclohexylmethyl)-1 H-pyrrolo[2,3 -b]pyridin-4 -yl)-1 H-indazol-3 -amine
5 -(2 -(2 -cyclohexylethyl)-1 H-pyrrolo[2,3 -b]pyridin-4 -yl)-1 H-indazol-3 -amine
5 -(2 -benzyl-1 H-pyrrolo[2,3 -b]pyridin-4 -yl)-1 H-indazol-3 -amine
(4 -(3 -amino-1 H-indazol-5 -yl)-1 H-pyrrolo[2,3 -b]pyridin-2 -yl)methanol
2 -(4 -(3 -amino-1 H-indazol-5 -yl)-1 H-pyrrolo[2,3 -b]pyridin-2 -yl)propan-2 -ol
3 -(4 -(3 -amino-1 H-indazol-5 -yl)-1 H-pyrrolo[2,3 -b]pyridin-2 -yl)pentan-3 -ol
5 -(2 -(tert-butoxymethyl)-1 H-pyrrolo[2,3 -b]pyridin-4 -yl)-1 H-indazol-3 -amine
5 -(2 -(tetrahydro-2 H-pyran-4 -yl)-1 H-pyrrolo[2,3 -b]pyridin-4 -yl)-1 H-indazol-3 -amine
5 -(2 -(tetrahydro-2 H-pyran-2 -yl)-1 H-pyrrolo[2,3 -b]pyridin-4 -yl)-1 H-indazol-3 -amine
5 -(2 -((tetrahydro-2 H-pyran-4 -yl)methyl)-1 H-pyrrolo[2,3 -b]pyridin-4 -yl)-1 H-indazol-3 -amine
4 -(3 -amino-1 H-indazol-5 -yl)-1 H-pyrrolo[2,3 -b]pyridine-2 -carboxylic acid
methyl 4 -(3 -amino-1 H-indazol-5 -yl)-1 H-pyrrolo[2,3 -b]pyridine-2 -carboxylate
ethyl 4 -(3 -amino-1 H-indazol-5 -yl)-1 H-pyrrolo[2,3 -b]pyridine-2 -carboxylate
(4 -(3 -amino-1 H-indazol-5 -yl)-1 H-pyrrolo[2,3 -b]pyridin-2 -yl)(pyrrolidin-1 -yl)methanone
4 -(3 -amino-1 H-indazol-5 -yl)-N-cyclopentyl-1 H-pyrrolo[2,3 -b]pyridine-2 -carboxamide
4 -(3 -amino-1 H-indazol-5 -yl)-N-cyclohexyl-1 H-pyrrolo[2,3 -b]pyridine-2 -carboxamide
4 -(3 -amino-1 H-indazol-5 -yl)-N-isopentyl-1 H-pyrrolo[2,3 -b]pyridine-2 -carboxamide
4 -(3 -amino-1 H-indazol-5 -yl)-N-phenethyl-1 H-pyrrolo[2,3 -b]pyridine-2 -carboxamide
4 -(3 -amino-1 H-indazol-5 -yl)-N-(3 -phenylpropyl)-1 H-pyrrolo[2,3 -b]pyridine-2 -carboxamide
4 -(3 -amino-1 H-indazol-5 -yl)-N-(2 -methoxyethyl)-1 H-pyrrolo[2,3 -b]pyridine-2 -carboxamide
4 -(3 -amino-1 H-indazol-5 -yl)-N-(2 -aminoethyl)-1 H-pyrrolo[2,3 -b]pyridine-2 -carboxamide
4 -(3 -amino-1 H-indazol-5 -yl)-N-(2 -(dimethylamino)ethyl)-1 H-pyrrolo[2,3 -b]pyridine-2 -carboxamide
(4 -(3 -amino-1 H-indazol-5 -yl)-1 H-pyrrolo[2,3 -b]pyridin-2 -yl)(4 -methylpiperazin-1 -yl)methanone
4 -(3 -amino-1 H-indazol-5 -yl)-N-(2 -(piperidin-1 -yl)ethyl)-1 H-pyrrolo[2,3 -b]pyridine-2 -carboxamide
4 -(3 -amino-1 H-indazol-5 -yl)-N-(2 -(butyl(ethyl)amino)ethyl)-1 H-pyrrolo[2,3 -b]pyridine-2 -carboxamide
4 -(3 -amino-1 H-indazol-5 -yl)-N-(2 -(diisopropylamino)ethyl)-1 H-pyrrolo[2,3 -b]pyridine-2 -carboxamide
4 -(3 -amino-1 H-indazol-5 -yl)-N-(3 -(dimethylamino)propyl)-1 H-pyrrolo[2,3 -b]pyridine-2 -carboxamide
5 -(2 -((tert-butylamino)methyl)-1 H-pyrrolo[2,3 -b]pyridine-4 -yl)-1 H-indazol-3 -amine
5 -(2 -((isopentylamino)methyl)-1 H-pyrrolo[2,3 -b]pyridine-4 -yl)-1 H-indazol-3 -amine
5 -(2 -(piperidin-2 -yl)-1 H-pyrrolo[2,3 -b]pyridine-4 -yl)-1 H-indazol-3 -amine
5 -(2 -((cyclohexylamino)methyl)-1 H-pyrrolo[2,3 -b]pyridine-4 -yl)-1 H-indazol-3 -amine
5 -(2 -((phenylamino)methyl)-1 H-pyrrolo[2,3 -b]pyridine-4 -yl)-1 H-indazol-3 -amine
5 -(2 -(((2 -(benzyloxy)phenyl)amino)methyl)-1 H-pyrrolo[2,3 -b]pyridine-4 -yl)-1 H-indazol-3 -amine
5 -(2 -(((2 -methoxyethyl)amino)methyl)-1 H-pyrrolo[2,3 -b]pyridine-4 -yl)-1 H-indazol-3 -amine
N1 -((4 -(3 -amino-1 H-indazol-5 -yl)-1 H-pyrrolo[2,3 -b]pyridin-2 -yl)methyl)-N2,N2 -dimethylethane-1,2 -diamine
5 -(2 -(((3 -methoxypropyl)amino)methyl)-1 H-pyrrolo[2,3 -b]pyridine-4 -yl)-1 H-indazol-3 -amine
5 -(2 -(((3 -isopropoxypropyl)amino)methyl)-1 H-pyrrolo[2,3 -b]pyridine-4 -yl)-1 H-indazol-3 -amine
N1 -((4 -(3 -amino-1 H-indazol-5 -yl)-1 H-pyrrolo[2,3 -b]pyridin-2 -yl)methyl)-N3,N3 -dimethylpropane-1,3 -diamine
5 -(2 -((isopropyl(methyl)amino)methyl)-1 H-pyrrolo[2,3 -b]pyridine-4 -yl)-1 H-indazol-3 -amine
5 -(2 -(piperidin-1 -ylmethyl)-1 H-pyrrolo[2,3 -b]pyridine-4 -yl)-1 H-indazol-3 -amine
5 -(2 -((4,4 -difluoropiperidin-1 -yl)methyl)-1 H-pyrrolo[2,3 -b]pyridine-4 -yl)-1 H-indazol-3 -amine
5 -(2 -(morpholinomethyl)-1 H-pyrrolo[2,3 -b]pyridine-4 -yl)-1 H-indazol-3 -amine
5 -(2 -((4 -methylpiperazin-1 -yl)methyl)-1 H-pyrrolo[2,3 -b]pyridine-4 -yl)-1 H-indazol-3 -amine
5 -(2 -((4 -(tert-butyl)piperazin-1 -yl)methyl)-1 H-pyrrolo[2,3 -b]pyridin-4 -yl)-1 H-indazol-3 -amine
5 -(2 -(azepan-1 -ylmethyl)-1 H-pyrrolo[2,3 -b]pyridine-4 -yl)-1 H-indazol-3 -amine
5 -(2 -((4 -methyl-1,4 -diazepan-1 -yl)methyl)-1 H-pyrrolo[2,3 -b]pyridin-4 -yl)-1 H-indazol-3 -amine
5 -(2 -(2 -(piperidin-1 -yl)ethyl)-1 H-pyrrolo[2,3 -b]pyridine-4 -yl)-1 H-indazol-3 -amine
5 -(2 -(2 -morpholinoethyl)-1 H-pyrrolo[2,3 -b]pyridin-4 -yl)-1 H-indazol-3 -amine
5 -(2 -(3 -(piperidin-1 -yl)propyl)-1 H-pyrrolo[2,3 -b]pyridin-4 -yl)-1 H-indazol-3 -amine
5 -(2 -(3 -(cyclohexylamino)propyl)-1 H-pyrrolo[2,3 -b]pyridin-4 -yl)-1 H-indazol-3 -amine
5 -(2 -(3 -morpholinopropyl)-1 H-pyrrolo[2,3 -b]pyridin-4 -yl)-1 H-indazol-3 -amine
5 -(2 -(piperidin-4 -ylmethyl)-1 H-pyrrolo[2,3 -b]pyridin-4 -yl)-1 H-indazol-3 -amine
5 -(2 -((1 -benzylpiperidin-4 -yl)methyl)-1 H-pyrrolo[2,3 -b]pyridin-4 -yl)-1 H-indazol-3 -amine
5 -(2 -phenyl-1 H-pyrrolo[2,3 -b]pyridin-4 -yl)-1 H-indazol-3 -amine
5 -(2 -(3 -(trifluoromethyl)phenyl)-1 H-pyrrolo[2,3 -b]pyridin-4 -yl)-1 H-indazol-3 -amine
3 -(4 -(3 -amino-1 H-indazol-5 -yl)-1 H-pyrrolo[2,3 -b]pyridin-2 -yl)benzonitrile
3 -(4 -(3 -amino-1 H-indazol-5 -yl)-1 H-pyrrolo[2,3 -b]pyridin-2 -yl)phenol
5 -(2 -(3 -methoxyphenyl)-1 H-pyrrolo[2,3 -b]pyridin-4 -yl)-1 H-indazol-3 -amine
3 -(4 -(3 -amino-1 H-indazol-5 -yl)-1 H-pyrrolo[2,3 -b]pyridin-2 -yl)benzoic acid
3 -(4 -(3 -amino-1 H-indazol-5 -yl)-1 H-pyrrolo[2,3 -b]pyridin-2 -yl)benzamide
3 -(4 -(3 -amino-1 H-indazol-5 -yl)-1 H-pyrrolo[2,3 -b]pyridin-2 -yl)-N-(2 -methoxyethyl)benzamide
3 -(4 -(3 -amino-1 H-indazol-5 -yl)-1 H-pyrrolo[2,3 -b]pyridin-2 -yl)-N-(2 -(piperidin-1 -yl)ethyl)benzamide
(3 -(4 -(3 -amino-1 H-indazol-5 -yl)-1 H-pyrrolo[2,3 -b]pyridin-2 -yl)phenyl)(4 -methylpiperazin-1 -yl)methanone
5 -(2 -(3 -(morpholinomethyl)phenyl)-1 H-pyrrolo[2,3 -b]pyridin-4 -yl)-1 H-indazol-3 -amine
5 -(2 -(3 -(methylsulfonyl)phenyl)-1 H-pyrrolo[2,3 -b]pyridin-4 -yl)-1 H-indazol-3 -amine
5 -(2 -(3 -(morpholinosulfonyl)phenyl)-1 H-pyrrolo[2,3 -b]pyridin-4 -yl)-1 H-indazol-3 -amine
5 -(2 -(3 -aminophenyl)-1 H-pyrrolo[2,3 -b]pyridin-4 -yl)-1 H-indazol-3 -amine
N-(3 -(4 -(3 -amino-1 H-indazol-5 -yl)-1 H-pyrrolo[2,3 -b]pyridin-2 -yl)phenyl)-3 -methoxypropanamide
N-(3 -(4 -(3 -amino-1 H-indazol-5 -yl)-1 H-pyrrolo[2,3 -b]pyridin-2 -yl)phenyl)-3 -(piperidin-1 -yl)propanamide
4 -((3 -(4 -(3 -amino-1 H-indazol-5 -yl)-1 H-pyrrolo[2,3 -b]pyridin-2 -yl)phenyl)amino)-4 -oxobutanoic acid
N-(3 -(4 -(3 -amino-1 H-indazol-5 -yl)-1 H-pyrrolo[2,3 -b]pyridin-2 -yl)phenyl)methanesulfonamide
N-(3 -(4 -(3 -amino-1 H-indazol-5 -yl)-1 H-pyrrolo[2,3 -b]pyridin-2 -yl)phenyl)-4 -methylbenzenesulfonamide
5 -(2 -(4 -(morpholinomethyl)phenyl)-1 H-pyrrolo[2,3 -b]pyridin-4 -yl)-1 H-indazol-3 -amine
N-(4 -(4 -(3 -amino-1 H-indazol-5 -yl)-1 H-pyrrolo[2,3 -b]pyridin-2 -yl)phenyl)-4 -methylbenzenesulfonamide
2 -(4 -(3 -amino-1 H-indazol-5 -yl)-1 H-pyrrolo[2,3 -b]pyridin-2 -yl)phenol
5 -(2 -(2 -ethoxyphenyl)-1 H-pyrrolo[2,3 -b]pyridin-4 -yl)-1 H-indazol-3 -amine
5 -(2 -(2 -ethylphenyl)-1 H-pyrrolo[2,3 -b]pyridin-4 -yl)-1 H-indazol-3 -amine
5 -(2 -(benzo[d][1.3 ]dioxol-5 -yl)-1 H-pyrrolo[2,3 -b]pyridin-4 -yl)-1 H-indazol-3 -amine
4 -(4 -(3 -amino-1 H-indazol-5 -yl)-1 H-pyrrolo[2,3 -b]pyridin-2 -yl)-2 -fluorobenzonitrile
5 -(4 -(3 -amino-1 H-indazol-5 -yl)-1 H-pyrrolo[2,3 -b]pyridin-2 -yl)-2 -(pyrrolidin-1 -yl)benzonitrile
5 -(4 -(3 -amino-1 H-indazol-5 -yl)-1 H-pyrrolo[2,3 -b]pyridin-2 -yl)-2 -(4 -methylpiperazin-1 -yl)benzonitrile
5,5 ′-(1 H-pyrrolo[2,3 -b]pyridine-2,4 -diyl)bis(1 H-indazol-3 -amine)
5 -(2 -(3,5 -difluorophenyl)-1 H-pyrrolo[2,3 -b]pyridin-4 -yl)-1 H-indazol-3 -amine
5 -(2 -(2,3,5 -trifluorophenyl)-1 H-pyrrolo[2,3 -b]pyridin-4 -yl)-1 H-indazol-3 -amine
5 -(2 -(pyridin-2 -yl)-1 H-pyrrolo[2,3 -b]pyridin-4 -yl)-1 H-indazol-3 -amine
5 -(2 -(pyridin-3 -yl)-1 H-pyrrolo[2,3 -b]pyridin-4 -yl)-1 H-indazol-3 -amine
5 -(2 -(pyridin-4 -yl)-1 H-pyrrolo[2,3 -b]pyridin-4 -yl)-1 H-indazol-3 -amine
5 -(2 -(pyrimidin-5 -yl)-1 H-pyrrolo[2,3 -b]pyridin-4 -yl)-1 H-indazol-3 -amine
4 -(4 -(3 -amino-1 H-indazol-5 -yl)-1 H-pyrrolo[2,3 -b]pyridin-2 -yl)pyridin-2 (1 H)-one
5 -(2 -(2 -fluoropyridin-4 -yl)-1 H-pyrrolo[2,3 -b]pyridin-4 -yl)-1 H-indazol-3 -amine
5 -(2 -(2 -fluoro-6 -methylpyridin-4 -yl)-1 H-pyrrolo[2,3 -b]pyridin-4 -yl)-1 H-indazol-3 -amine
5 -(2 -(2,6 -difluoropyridin-4 -yl)-1 H-pyrrolo[2,3 -b]pyridin-4 -yl)-1 H-indazol-3 -amine
5 -(2 -(2 -(piperidin-1 -yl)pyridin-4 -yl)-1 H-pyrrolo[2,3 -b]pyridin-4 -yl)-1 H-indazol-3 -amine
5 -(2 -(2 -(piperazin-1 -yl)pyridin-4 -yl)-1 H-pyrrolo[2,3 -b]pyridin-4 -yl)-1 H-indazol-3 -amine
5 -(2 -(2 -morpholinopyridin-4 -yl)-1 H-pyrrolo[2,3 -b]pyridin-4 -yl)-1 H-indazol-3 -amine
5 -(2 -(2 -(4 -(tert-butyl)piperazin-1 -yl)pyridin-4 -yl)-1 H-pyrrolo[2,3 -b]pyridin-4 -yl)-1 H-indazol-3 -amine
4 -(4 -(4 -(3 -amino-1 H-indazol-5 -yl)-1 H-pyrrolo[2,3 -b]pyridin-2 -yl)pyridin-2 -yl)thiomorpholine 1,1 -dioxide
5 -(2 -(2,6 -dimorpholinopyridin-4 -yl)-1 H-pyrrolo[2,3 -b]pyridin-4 -yl)-1 H-indazol-3 -amine
4 -(4 -(3 -amino-1 H-indazol-5 -yl)-1 H-pyrrolo[2,3 -b]pyridin-2 -yl)-1 -(4 -(piperidin-1 -ylmethyl)benzyl)pyridin-2 (1 H)-one
4 -(4 -(3 -amino-1 H-indazol-5 -yl)-1 H-pyrrolo[2,3 -b]pyridin-2 -yl)-1 -(3 -(piperidin-1 -ylmethyl)benzyl)pyridin-2 (1 H)-one
4 -(4 -(3 -amino-1 H-indazol-5 -yl)-1 H-pyrrolo[2,3 -b]pyridin-2 -yl)-1 -(2 -(piperidin-1 -ylmethyl)benzyl)pyridin-2 (1 H)-one
5 -(2 -(5 -methoxypyridin-3 -yl)-1 H-pyrrolo[2,3 -b]pyridin-4 -yl)-1 H-indazol-3 -amine
5 -(2 -(6 -morpholinopyridin-3 -yl)-1 H-pyrrolo[2,3 -b]pyridin-4 -yl)-1 H-indazol-3 -amine
5 -(2 -(6 -(4 -methylpiperazin-1 -yl)pyridin-3 -yl)-1 H-pyrrolo[2,3 -b]pyridin-4 -yl)-1 H-indazol-3 -amine
5 -(2 -(6 -((2 -morpholinoethyl)amino)pyridin-3 -yl)-1 H-pyrrolo[2,3 -b]pyridin-4 -yl)-1 H-indazol-3 -amine
5 -(2 -(2 -fluoropyridin-3 -yl)-1 H-pyrrolo[2,3 -b]pyridin-4 -yl)-1 H-indazol-3 -amine
5 -(2 -(2 -morpholinopyridin-3 -yl)-1 H-pyrrolo[2,3 -b]pyridin-4 -yl)-1 H-indazol-3 -amine
5 -(2 -(3 -methoxyphenyl)-1 H-pyrrolo[2,3 -b]pyridin-4 -yl)-1 H-indazol-3 -amine
5 -(2 -(3 -isobutoxyphenyl)-1 H-pyrrolo[2,3 -b]pyridin-4 -yl)-1 H-indazol-3 -amine
5 -(2 -(3 -((tetrahydro-2 H-pyran-4 -yl)methoxy)phenyl)-1 H-pyrrolo[2,3 -b]pyridin-4 -yl)-1 H-indazol-3 -amine
5 -(2 -(3 -(2 -morpholinoethoxy)phenyl)-1 H-pyrrolo[2,3 -b]pyridin-4 -yl)-1 H-indazol-3 -amine
5 -(2 -(3 -(benzyloxy)phenyl)-1 H-pyrrolo[2,3 -b]pyridin-4 -yl)-1 H-indazol-3 -amine
5 -(2 -(3 -((4 -methoxybenzyl)oxy)phenyl)-1 H-pyrrolo[2,3 -b]pyridin-4 -yl)-1 H-indazol-3 -amine
5 -(2 -(3 -((3,5 -dimethoxybenzyl)oxy)phenyl)-1 H-pyrrolo[2,3 -b]pyridin-4 -yl)-1 H-indazol-3 -amine
5 -(2 -(3 -((4 -fluorobenzyl)oxy)phenyl)-1 H-pyrrolo[2,3 -b]pyridin-4 -yl)-1 H-indazol-3 -amine
5 -(2 -(3 -((2 -fluorobenzyl)oxy)phenyl)-1 H-pyrrolo[2,3 -b]pyridin-4 -yl)-1 H-indazol-3 -amine
5 -(2 -(3 -(pyridin-2 -ylmethoxy)phenyl)-1 H-pyrrolo[2,3 -b]pyridin-4 -yl)-1 H-indazol-3 -amine
3 -(4 -(3 -amino-1 H-indazol-5 -yl)-1 H-pyrrolo[2,3 -b]pyridin-2 -yl)-5 -fluorophenol
5 -(2 -(3 -fluoro-5 -methoxyphenyl)-1 H-pyrrolo[2,3 -b]pyridin-4 -yl)-1 H-indazol-3 -amine
5 -(2 -(3 -fluoro-5 -(2 -methoxyethoxy)phenyl)-1 H-pyrrolo[2,3 -b]pyridine-4 -yl)-1 H-indazol-3 -amine
5 -(2 -(3 -fluoro-5 -((tetrahydro-2 H-pyran-4 -yl)methoxy)phenyl)-1 H-pyrrolo[2,3 -b]pyridine-4 -yl)-1 H-indazol-3 -amine
5 -(2 -(3 -(benzyloxy)-5 -fluorophenyl)-1 H-pyrrolo[2,3 -b]pyridin-4 -yl)-1 H-indazol-3 -amine
5 -(2 -(3 -(benzyloxy)-5 -(trifluoromethoxy)phenyl)-1 H-pyrrolo[2,3 -b]pyridin-4 -yl)-1 H-indazol-3 -amine
methyl 3 -(4 -(3 -amino-1 H-indazol-5 -yl)-1 H-pyrrolo[2,3 -b]pyridin-2 -yl)-5 -(benzyloxy)benzoate
5 -(2 -(3 -(benzyloxy)-5 -((2 -methoxyethoxy)methyl)phenyl)-1 H-pyrrolo[2,3 -b]pyridin-4 -yl)-1 H-indazol-3 -amine
5 -(2 -(3 -(benzyloxy)-5 -((2 -methoxyethyl)amino)phenyl)-1 H-pyrrolo[2,3 -b]pyridin-4 -yl)-1 H-indazol-3 -amine
5 -(2 -(3 -(benzyloxy)-5 -((2 -morpholinoethyl)amino)phenyl)-1 H-pyrrolo[2,3 -b]pyridin-4 -yl)-1 H-indazol-3 -amine
5 -(2 -(2 -(benzyloxy)phenyl)-1 H-pyrrolo[2,3 -b]pyridin-4 -yl)-1 H-indazol-3 -amine
5 -(2 -(4 -(benzyloxy)phenyl)-1 H-pyrrolo[2,3 -b]pyridin-4 -yl)-1 H-indazol-3 -amine
5 -(2 -(5 -(benzyloxy)pyridin-3 -yl)-1 H-pyrrolo[2,3 -b]pyridin-4 -yl)-1 H-indazol-3 -amine
5 -(2 -(2 -(benzyloxy)pyridin-4 -yl)-1 H-pyrrolo[2,3 -b]pyridin-4 -yl)-1 H-indazol-3 -amine
5 -(2 -(6 -(benzyloxy)pyridin-3 -yl)-1 H-pyrrolo[2,3 -b]pyridin-4 -yl)-1 H-indazol-3 -amine
5 -(2 -(4 -(benzyloxy)pyridin-2 -yl)-1 H-pyrrolo[2,3 -b]pyridin-4 -yl)-1 H-indazol-3 -amine
5 -(2 -(2 -(pyrimidin-5 -ylmethoxy)pyridin-4 -yl)-1 H-pyrrolo[2,3 -b]pyridin-4 -yl)-1 H-indazol-3 -amine
5 -(2 -(2 -(pyridin-4 -ylmethoxy)pyridin-4 -yl)-1 H-pyrrolo[2,3 -b]pyridin-4 -yl)-1 H-indazol-3 -amine
5 -(2 -(2 -(pyridin-3 -ylmethoxy)pyridin-4 -yl)-1 H-pyrrolo[2,3 -b]pyridin-4 -yl)-1 H-indazol-3 -amine
5 -(2 -(2 -(pyridin-2 -ylmethoxy)pyridin-4 -yl)-1 H-pyrrolo[2,3 -b]pyridin-4 -yl)-1 H-indazol-3 -amine
5 -(2 -(6 -(benzylamino)pyridin-3 -yl)-1 H-pyrrolo[2,3 -b]pyridin-4 -yl)-1 H-indazol-3 -amine
5 -(2 -(2 -(benzylamino)pyridin-4 -yl)-1 H-pyrrolo[2,3 -b]pyridin-4 -yl)-1 H-indazol-3 -amine
5 -(2 -(2 -(benzyl(methyl)amino)pyridin-4 -yl)-1 H-pyrrolo[2,3 -b]pyridin-4 -yl)-1 H-indazol-3 -amine
5 -(2 -(2 -(benzylthio)pyridin-4 -yl)-1 H-pyrrolo[2,3 -b]pyridin-4 -yl)-1 H-indazol-3 -amine
5 -(2 -(2 -(benzylthio)-6 -morpholinopyridin-4 -yl)-1 H-pyrrolo[2,3 -b]pyridin-4 -yl)-1 H-indazol-3 -amine
5 -(2 -(3 -phenethylphenyl)-1 H-pyrrolo[2,3 -b]pyridin-4 -yl)-1 H-indazol-3 -amine
5 -(2 -(2 -phenethoxypyridin-4 -yl)-1 H-pyrrolo[2,3 -b]pyridin-4 -yl)-1 H-indazol-3 -amine
5 -(2 -(3 -((phenylamino)methyl)phenyl)-1 H-pyrrolo[2,3 -b]pyridin-4 -yl)-1 H-indazol-3 -amine
5 -(2 -(3 -((tert-butylamino)methyl)phenyl)-1 H-pyrrolo[2,3 -b]pyridin-4 -yl)-1 H-indazol-3 -amine
5 -(2 -(3 -((cyclopentylamino)methyl)phenyl)-1 H-pyrrolo[2,3 -b]pyridin-4 -yl)-1 H-indazol-3 -amine
5 -(2 -(3 -((cyclohexylamino)methyl)phenyl)-1 H-pyrrolo[2,3 -b]pyridin-4 -yl)-1 H-indazol-3 -amine
5 -(2 -(3 -((butylamino)methyl)phenyl)-1 H-pyrrolo[2,3 -b]pyridin-4 -yl)-1 H-indazol-3 -amine
5 -(2 -(3 -((isopentylamino)methyl)phenyl)-1 H-pyrrolo[2,3 -b]pyridin-4 -yl)-1 H-indazol-3 -amine
5 -(2 -(3 -((butyl(ethyl)amino)methyl)phenyl)-1 H-pyrrolo[2,3 -b]pyridin-4 -yl)-1 H-indazol-3 -amine
5 -(2 -(3 -((dibutylamino)methyl)phenyl)-1 H-pyrrolo[2,3 -b]pyridin-4 -yl)-1 H-indazol-3 -amine
5 -(2 -(2 -fluoro-6 -(piperidin-1 -ylmethyl)pyridin-4 -yl)-1 H-pyrrolo[2,3 -b]pyridin-4 -yl)-1 H-indazol-3 -amine
5 -(2 -(2 -fluoro-6 -(piperazin-1 -ylmethyl)pyridin-4 -yl)-1 H-pyrrolo[2,3 -b]pyridin-4 -yl)-1 H-indazol-3 -amine
5 -(2 -(2 -fluoro-6 -((4 -methylpiperazin-1 -yl)methyl)pyridin-4 -yl)-1 H-pyrrolo[2,3 -b]pyridin-4 -yl)-1 H-indazol-3 -amine
tert-butyl 4 -((4 -(4 -(3 -amino-1 H-indazol-5 -yl)-1 H-pyrrolo[2,3 -b]pyridin-2 -yl)-6 -fluoropyridin-2 -yl)methyl)piperazine-1 -carboxylate
5 -(2 -(2 -((tert-butylamino)methyl)-6 -fluoropyridin-4 -yl)-1 H-pyrrolo[2,3 -b]pyridin-4 -yl)-1 H-indazol-3 -amine
5 -(2 -(2 -((cyclohexylamino)methyl)-6 -fluoropyridin-4 -yl)-1 H-pyrrolo[2,3 -b]pyridin-4 -yl)-1 H-indazol-3 -amine
5 -(2 -(2 -fluoro-6 -((phenylamino)methyl)pyridin-4 -yl)-1 H-pyrrolo[2,3 -b]pyridin-4 -yl)-1 H-indazol-3 -amine
4 -(4 -(3 -amino-1 H-indazol-5 -yl)-1 H-pyrrolo[2,3 -b]pyridin-2 -yl)-2,6 -difluorobenzamide
5 -(2 -(4 -((dimethylamino)methyl)-3,5 -difluorophenyl)-1 H-pyrrolo[2,3 -b]pyridin-4 -yl)-1 H-indazol-3 -amine
5 -(2 -(3,5 -difluoro-4 -(piperidin-1 -ylmethyl)phenyl)-1 H-pyrrolo[2,3 -b]pyridin-4 -yl)-1 H-indazol-3 -amine
5 -(2 -(3,5 -difluoro-4 -((isopropyl(methyl)amino)methyl)phenyl)-1 H-pyrrolo[2,3 -b]pyridin-4 -yl)-1 H-indazol-3 -amine
5 -(2 -(4 -((butyl(ethyl)amino)methyl)-3,5 -difluorophenyl)-1 H-pyrrolo[2,3 -b]pyridin-4 -yl)-1 H-indazol-3 -amine
5 -(2 -(4 -((dibutylamino)methyl)-3,5 -difluorophenyl)-1 H-pyrrolo[2,3 -b]pyridin-4 -yl)-1 H-indazol-3 -amine
5 -(2 -(3,5 -difluoro-4 -(3 -morpholinopropyl)phenyl)-1 H-pyrrolo[2,3 -b]pyridin-4 -yl)-1 H-indazol-3 -amine
5 -(2 -(3,5 -difluoro-4 -(3 -(piperidin-1 -yl)propyl)phenyl)-1 H-pyrrolo[2,3 -b]pyridin-4 -yl)-1 H-indazol-3 -amine
5 -(2 -(4 -(3 -(diethylamino)propyl)-3,5 -difluorophenyl)-1 H-pyrrolo[2,3 -b]pyridin-4 -yl)-1 H-indazol-3 -amine
5 -(2 -(4 -(3 -(dibutylamino)propyl)-3,5 -difluorophenyl)-1 H-pyrrolo[2,3 -b]pyridin-4 -yl)-1 H-indazol-3 -amine
7 -chloro-5 -(2 -phenyl-1 H-pyrrolo[2,3 -b]pyridin-4 -yl)-1 H-indazol-3 -amine
7 -chloro-5 -(2 -(3 -(morpholinomethyl)phenyl)-1 H-pyrrolo[2,3 -b]pyridin-4 -yl)-1 H-indazol-3 -amine
7 -chloro-5 -(2 -(4 -(morpholinomethyl)phenyl)-1 H-pyrrolo[2,3 -b]pyridin-4 -yl)-1 H-indazol-3 -amine
7 -chloro-5 -(2 -(2 -fluoro-6 -(piperazin-1 -ylmethyl)pyridin-4 -yl)-1 H-pyrrolo[2,3 -b]pyridin-4 -yl)-1 H-indazol-3 -amine
4 -(3 -amino-7 -chloro-1 H-indazol-5 -yl)-N-(2 -(piperidin-1 -yl)ethyl)-1 H-pyrrolo[2,3 -b]pyridine-2 -carboxamide
7 -chloro-5 -(2 -(cyclohexylmethyl)-1 H-pyrrolo[2,3 -b]pyridin-4 -yl)-1 H-indazol-3 -amine
7 -chloro-5 -(2 -(morpholinomethyl)-1 H-pyrrolo[2,3 -b]pyridin-4 -yl)-1 H-indazol-3 -amine
7 -chloro-5 -(2 -(2 -morpholinoethyl)-1 H-pyrrolo[2,3 -b]pyridin-4 -yl)-1 H-indazol-3 -amine
7 -phenyl-5 -(2 -phenyl-1 H-pyrrolo[2,3 -b]pyridin-4 -yl)-1 H-indazol-3 -amine
5 -(2 -(3 -(morpholinomethyl)phenyl)-1 H-pyrrolo[2,3 -b]pyridin-4 -yl)-7 -phenyl-1 H-indazol-3 -amine
5 -(2 -(4 -(morpholinomethyl)phenyl)-1 H-pyrrolo[2,3 -b]pyridin-4 -yl)-7 -phenyl-1 H-indazol-3 -amine
5 -(2 -(cyclohexylmethyl)-1 H-pyrrolo[2,3 -b]pyridin-4 -yl)-7 -(3,3 -dimethylbut-1 -yn-1 -yl)-1 H-indazol-3 -amine
5 -(2 -(2 -(Benzylthio)-6 -fluoropyridin-4 -yl)-1 H-pyrrolo[2,3 -b]pyridin-4 -yl)-1 H-indazol-3 -amine.

The various functional groups and substituents making up the compounds of the Formula (I), or sub-formulae (Ia) to (Ic), are typically chosen such that the molecular weight of the compound of the formula (I) does not exceed 1000 . More usually, the molecular weight of the compound will be less than 900, for example less than 800, or less than 750, or less than 700, or less than 650 . More preferably, the molecular weight is less than 600 and, for example, is 550 or less.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulfuric, phosphoric, trifluoroacetic, formic, citric methane sulfonate or maleic acid. In addition, a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a pharmaceutically acceptable cation, for example a salt with methylamine, dimethylamine, trimethylamine, piperidine, morpholine or tris-(2 -hydroxyethyl)amine.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this invention may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4 th edition J. March, John Wiley and Sons, New York, 2001 ), for example by synthesis from optically active starting materials or by resolution of a racemic form. Some of the compounds of the invention may have geometric isomeric centres (E- and Z-isomers).

It is to be understood that the present invention encompasses all optical, diastereoisomers and geometric isomers and mixtures thereof that possess activity.

The present invention also encompasses compounds of the invention as defined herein which comprise one or more isotopic substitutions. For example, H may be in any isotopic form, including 1 H, 2 H(D), and 3 H (T); C may be in any isotopic form, including 12 C, 13 C, and 14 C; and O may be in any isotopic form, including 160 and180 ; and the like.

It is also to be understood that certain compounds of the Formula (I), or sub-formulae (Ia) to (Ic), may exist in solvated as well as unsolvated forms such as, for example, hydrated forms. It is to be understood that the invention encompasses all such solvated forms that possess activity.

It is also to be understood that certain compounds of the Formula (I), or sub-formulae (Ia) to (Ic), may exhibit polymorphism, and that the invention encompasses all such forms that possess activity.

Compounds of the Formula (I), or sub-formulae (Ia) to (Ic), may exist in a number of different tautomeric forms and references to compounds of the Formula (I), or sub-formulae (Ia) to (Ic), include all such forms. For the avoidance of doubt, where a compound can exist in one of several tautomeric forms, and only one is specifically described or shown, all others are nevertheless embraced by Formula (I), or sub-formulae (Ia) to (Ic). Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

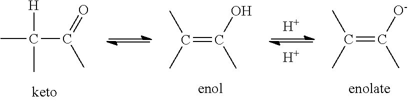

Compounds of the Formula (I), or sub-formulae (Ia) to (Ic), containing an amine function may also form N-oxides. A reference herein to a compound of the Formula (I), or sub-formulae (Ia) to (Ic), that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4 th Edition, Wiley Interscience, pages. More particularly, N-oxides can be made by the procedure of L. W. Deady (Syn. Comm. 1977, 7, 509-514 ) in which the amine compound is reacted with m-chloroperoxybenzoic acid (mCPBA), for example, in an inert solvent such as dichloromethane.

The compounds of Formula (I), or sub-formulae (Ia) to (Ic), may be administered in the form of a pro-drug which is broken down in the human or animal body to release a compound of the invention. A pro-drug may be used to alter the physical properties and/or the pharmacokinetic properties of a compound of the invention. A pro-drug can be formed when the compound of the invention contains a suitable group or substituent to which a property-modifying group can be attached. Examples of pro-drugs include in vivo cleavable ester derivatives that may be formed at a carboxy group or a hydroxy group in a compound of the Formula (I), or sub-formulae (Ia) to (Ic), and in-vivo cleavable amide derivatives that may be formed at a carboxy group or an amino group in a compound of the Formula (I), or sub-formulae (Ia) to (Ic).

Accordingly, the present invention includes those compounds of the Formula (I), or sub-formulae (Ia) to (Ic), as defined hereinbefore, when made available by organic synthesis and when made available within the human or animal body by way of cleavage of a pro-drug thereof. Accordingly, the present invention includes those compounds of the Formula (I), or sub-formulae (Ia) to (Ic), that are produced by organic synthetic means and also such compounds that are produced in the human or animal body by way of metabolism of a precursor compound, that is a compound of the Formula (I), or sub-formulae (Ia) to (Ic), may be a synthetically-produced compound or a metabolically-produced compound.

A suitable pharmaceutically acceptable pro-drug of a compound of the Formula (I), or sub-formulae (Ia) to (Ic), is one that is based on reasonable medical judgement as being suitable for administration to the human or animal body without undesirable pharmacological activities and without undue toxicity.

Various forms of pro-drug have been described, for example in the following documents:—
a) Methods in Enzymology, Vol. 42, p. 309-396, edited by K. Widder, et al. (Academic Press, 1985 );
b) Design of Pro-drugs, edited by H. Bundgaard, (Elsevier, 1985 );
c) A Textbook of Drug Design and Development, edited by Krogsgaard-Larsen and H. Bundgaard, Chapter 5 “Design and Application of Pro-drugs”, by H. Bundgaard p. 113-191
(1991 );
d) H. Bundgaard, Advanced Drug Delivery Reviews, 8, 1-38 (1992 );
e) H. Bundgaard, et al., Journal of Pharmaceutical Sciences, 77, 285 (1988 );
f) N. Kakeya, et al., Chem. Pharm. Bull., 32, 692 (1984 );
g) T. Higuchi and V. Stella, “Pro-Drugs as Novel Delivery Systems”, A.C.S. Symposium Series, Volume 14 ; and
h) E. Roche (editor), “Bioreversible Carriers in Drug Design”, Pergamon Press, 1987.

A suitable pharmaceutically acceptable pro-drug of a compound of the Formula (I), or sub-formulae (Ia) to (Ic), that possesses a carboxy group is, for example, an in vivo cleavable ester thereof. An in vivo cleavable ester of a compound of the Formula I, or sub-formulae (Ia) to (Ic), containing a carboxy group is, for example, a pharmaceutically acceptable ester which is cleaved in the human or animal body to produce the parent acid or parent alcohol. Suitable pharmaceutically acceptable esters for carboxy include (1-6 C)alkyl esters such as methyl, ethyl and tert-butyl, (1-6 C)alkoxymethyl esters such as methoxymethyl esters, (1 -6 C)alkanoyloxymethyl esters such as pivaloyloxymethyl esters, 3 -phthalidyl esters, (3 -8 C)cycloalkylcarbonyloxy-(1-6 C)alkyl esters such as cyclopentylcarbonyloxymethyl and 1 -cyclohexylcarbonyloxyethyl esters, 2 -oxo-1,3 -dioxolenylmethyl esters such as 5 -methyl-2 -oxo-1,3 -dioxolen-4 -ylmethyl esters and (1-6 C)alkoxycarbonyloxy-(1-6 C)alkyl esters such as methoxycarbonyloxymethyl and 1 -methoxycarbonyloxyethyl esters.

A suitable pharmaceutically acceptable pro-drug of a compound of the Formula (I), or sub-formulae (Ia) to (Ic), that possesses a hydroxy group is, for example, an in vivo cleavable ester or ether thereof. An in vivo cleavable ester or ether of a compound of the Formula (I), or sub-formulae (Ia) to (Ic), containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include (1-10 C)alkanoyl groups such as acetyl, benzoyl, phenylacetyl and substituted benzoyl and phenylacetyl groups, (1-10 C)alkoxycarbonyl groups such as ethoxycarbonyl, N,N-(1 -6 C)₂ carbamoyl, 2 -dialkylaminoacetyl and 2 -carboxyacetyl groups. Examples of ring substituents on the phenylacetyl and benzoyl groups include aminomethyl, N-alkylaminomethyl, N,N-dialkylaminomethyl, morpholinomethyl, piperazin-1 -ylmethyl and 4 -(1-4 C)alkylpiperazin-1 -ylmethyl. Suitable pharmaceutically acceptable ether forming groups for a hydroxy group include a-acyloxyalkyl groups such as acetoxymethyl and pivaloyloxymethyl groups.

A suitable pharmaceutically acceptable pro-drug of a compound of the Formula (I), or sub-formulae (Ia) to (Ic), that possesses a carboxy group is, for example, an in vivo cleavable amide thereof, for example an amide formed with an amine such as ammonia, a (1 -4 C)alkylamine such as methylamine, a [(1-4 C)alkyl]₂ amine such as dimethylamine, N-ethyl-N-methylamine or diethylamine, a (1-4 C)alkoxy-(2-4 C)alkylamine such as 2 -methoxyethylamine, a phenyl-(1-4 C)alkylamine such as benzylamine and amino acids such as glycine or an ester thereof.

A suitable pharmaceutically acceptable pro-drug of a compound of the Formula (I), or sub-formulae (Ia) to (Ic), that possesses an amino group is, for example, an in vivo cleavable amide derivative thereof. Suitable pharmaceutically acceptable amides from an amino group include, for example an amide formed with (1-10 C)alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1 -ylmethyl and 4 -(1-4 C)alkyl)piperazin-1 -ylmethyl.

The in vivo effects of a compound of the Formula (I), or sub-formulae (Ia) to (Ic), may be exerted in part by one or more metabolites that are formed within the human or animal body after administration of a compound of the Formula (I), or sub-formulae (Ia) to (Ic). As stated hereinbefore, the in vivo effects of a compound of the Formula (I), or sub-formulae (Ia) to (Ic), may also be exerted by way of metabolism of a precursor compound (a pro-drug).

Though the present invention may relate to any compound or particular group of compounds defined herein by way of optional, preferred or suitable features or otherwise in terms of particular embodiments, the present invention may also relate to any compound or particular group of compounds that specifically excludes said optional, preferred or suitable features or particular embodiments.

Suitably, the present invention excludes any individual compounds not possessing the biological activity defined herein.

### Synthesis

The compounds of the present invention can be prepared by any suitable technique known in the art. Particular processes for the preparation of these compounds are described further in the accompanying examples.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilised.

It will be appreciated that during the synthesis of the compounds of the invention in the processes defined herein, or during the synthesis of certain starting materials, it may be desirable to protect certain substituent groups to prevent their undesired reaction. The skilled chemist will appreciate when such protection is required, and how such protecting groups may be put in place, and later removed.

For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule.

Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide.

Alternatively an acyl group such as a tert-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

Resins may also be used as a protecting group.

The methodology employed to synthesise a compound of Formula (I), or sub-formulae (Ia) to (Ic), will vary depending on the nature of R¹, R², R³, R⁴, X₁ and any substituent groups or subgroups associated therewith. Suitable processes for their preparation are described further in the accompanying Examples.

Once a compound of Formula (I), or sub-formulae (Ia) to (Ic), has been synthesised by any one of the processes defined herein, the processes may then further comprise the additional steps of:
(i) removing any protecting groups present;
(ii) converting the compound Formula (I) into another compound of Formula (I);
(iii) forming a pharmaceutically acceptable salt, hydrate or solvate thereof; and/or
(iv) forming a prodrug thereof.

An example of (ii) above is when a compound of Formula (I) is synthesised and then one or more of the groups may be further reacted to change the nature of the group and provide an alternative compound of Formula (I).

The resultant compounds of Formula (I), or sub-formulae (Ia) to (Ic), can be isolated and purified using techniques well known in the art.

The compounds of Formula (I) may be synthesised by the synthetic routes shown in the Examples section below.

### Biological Activity

The biological assays described in the Examples section herein may be used to measure the pharmacological effects of the compounds of the present invention.

Although the pharmacological properties of the compounds of Formula (I) vary with structural change, as expected, the compounds of the invention were found to be active in the IKK-alpha in vitro assay described in the Examples section, with preferred compounds showing selectivity for IKK-alpha over IKK-beta.

### Pharmaceutical Compositions

According to a further aspect of the invention there is provided a pharmaceutical composition which comprises a compound of the invention as defined hereinbefore, or a pharmaceutically acceptable salt, hydrate or solvate thereof, in association with a pharmaceutically acceptable diluent or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

An effective amount of a compound of the present invention for use in therapy is an amount sufficient to treat or prevent a proliferative condition referred to herein, slow its progression and/or reduce the symptoms associated with the condition.

The amount of active ingredient that is combined with one or more excipients to produce a single dosage form will necessarily vary depending upon the individual treated and the particular route of administration. For example, a formulation intended for oral administration to humans will generally contain, for example, from 0.5 mg to 0.5 g of active agent (more suitably from 0.5 to 100 mg, for example from 1 to 30 mg) compounded with an appropriate and convenient amount of excipients which may vary from about 5 to about 98 percent by weight of the total composition.

The size of the dose for therapeutic or prophylactic purposes of a compound of the formula I will naturally vary according to the nature and severity of the conditions, the age and sex of the animal or patient and the route of administration, according to well-known principles of medicine.

In using a compound of the invention for therapeutic or prophylactic purposes it will generally be administered so that a daily dose in the range, for example, 0.1 mg/kg to 75 mg/kg body weight is received, given if required in divided doses. In general lower doses will be administered when a parenteral route is employed. Thus, for example, for intravenous or intraperitoneal administration, a dose in the range, for example, 0.1 mg/kg to 30 mg/kg body weight will generally be used. Similarly, for administration by inhalation, a dose in the range, for example, 0.05 mg/kg to 25 mg/kg body weight will be used. Oral administration may also be suitable, particularly in tablet form. Typically, unit dosage forms will contain about 0.5 mg to 0.5 g of a compound of this invention.

### Therapeutic Uses and Applications

The present invention provides compounds that function as inhibitors of IKK activity, particularly IKKα activity. Accordingly, the compounds of the present invention are suitable for the treatment of any disease or condition in which the inhibition of IKKα activity is potentially beneficial.

IKKα activity is known to play a role in cancer.

### The Role of IKKα in Cancer

### IKKα in Solid Tumours

In recent years the role of the non-canonical NF-κB pathway and IKKα within it have increasingly been implicated in the development and progression of multiple solid tumours. The non-canonical NF-κB pathway has been associated with poor prognosis in glioblastoma [57 ] and mouse orthotopic models have demonstrated that up-regulation of this pathway is associated with an aggressive glioblastoma subtype [57 ]. In prostate cancer, nuclear localisation of RelB is associated with higher grade tumours [58 ] and treatment of prostate cancer cells with androgens induces accumulation of nuclear p52 [59 ]. In addition, silencing of IKKα reduces androgen receptor activity and gene expression, providing evidence that IKKα is associated with prostate cancer growth [58 ]. Therefore, IKKα is an attractive target for prostate cancer as the androgen receptor is the main driver of prostate cancer proliferation and inhibition of cell death.

In pancreatic cancer, the non-canonical NF-κB pathway is constitutively activated and associated with increased cell proliferation [60 ]. NIK is elevated in pancreatic cancer and associated with increased proliferation [61, 62 ] and up-regulation of RelB and p52 are associated with mutated KRAS pancreatic cancer [63 ] with IKKα-dependent gene expression being observed. In gastrointestinal tumours NF-κB2^{DCT/DCT} mice develop tumours spontaneously, providing evidence that p100 /p52 drives oncogenesis in this setting [64 ]. In renal cancer, members of the non-canonical NF-κB pathway are associated with poor prognosis, increased disease stage and decreased local inflammation [65 ]. In lung cancer, RelB is associated with shorter overall survival, differentiation, tumour invasion, lymph node metastasis, distant metastasis and ‘tumour, node, metastasis’ (TNM) stage [68 ]. In bladder cancer, up-regulation of RelB and p52 correlate with histological grade, stage and lymph node metastasis [69].

There are also numerous studies investigating IKKα in breast cancer. IKKα, RelB and p52 are associated with decreased cancer specific survival in ER-positive breast disease [70 , 71 ]. Bcl3 can form a DNA-binding complex with p52 and has been observed as over expressed in breast cancer samples. IKKα is demonstrated to play an essential role in the proliferation of mammary epithelium and it is therefore not surprising that aberrant IKKα signalling has been reported in breast cancer [72 ]. Yang et al. 2013 reports that in HER2 positive epithelial cells nuclear IKKα can promote progression to tumourogenesis via p27 [73 ]. In transgenic mice, overexpression of p100/52 results in a delay of mammary gland development, which is accompanied with over expression of cyclin D1, MMP2, MMP9 and COX-2 expression and results in the mice developing multiple tumours [74 ]. In addition, constitutive RANK signalling causes elevation of non-canonical NF-κB signalling in breast cancer cell lines, which subsequently stimulates cell proliferation via increased transcription of cyclin D1 [75-77 ] and nuclear IKKα expression is observed in invasive ductal carcinoma and associated with disease free survival. Immuno-histochemical studies have demonstrated that the p52 subunit is expressed at a higher level in the breast cancer tissue compared to normal adjacent tissue [78 ] and Western blots of nuclear fractions extracted from cancerous and adjacent normal breast tissue confirm an increase in p52 levels in the tumour cells [78 ]. This is accompanied by an increase in mRNA levels of p52, Bcl-3 and cyclin D1, all genes regulated by IKKα [78 ]. In addition, IKKα has been demonstrated in cervical, lung, prostate and pancreatic cell lines to regulate mTORC1 and mTORC2 which control tumour cell proliferation [79 ]. Taken as a whole, there is now a large body of evidence to support the role of the IKKα-NF-κB non-canonical pathway in the development and progression of solid tumours.

### IKKα Signalling Independent of NF-κB Pathways in Solid Tumours

In addition to the role IKKα plays in NF-κB pathways, it is also reported to have a role independent of both the canonical and non-canonical NF-κB pathways. IKKα accumulates in the nucleus, where it can phosphorylate a variety of substrates including histone H3, SMRT and nuclear co-repressor (NCoR) [80 ]. In colorectal cancer, IKKα phosphorylates SMRT, resulting in increased expression of Notch dependent genes [80 ]. In addition, IKKα has been reported to be associated with NOTCH activation in the presence of anti-oestrogens in breast cancer, resulting in up-regulation of ER-dependent gene expression and providing a mechanism for hormone resistance in an NF-κB independent manner [81, 82 ]. Bennett et al. reported that IKKα expression and not NIK or RelB is associated with recurrence in Luminal A breast cancer, suggesting it is independent of the non-canonical NF-κB pathway [71 ]. In a second cohort of patients who received tamoxifen, the authors reported that cytoplasmic IKKα was associated with disease-free survival and recurrence-free survival on tamoxifen in Luminal A disease, which may predict patients likely to develop resistance to tamoxifen or IKKα targeted therapies [71 ] again supporting a role for IKKα in tamoxifen resistant breast cancer. In contrast however, Roseweir et al. reported in the Tamoxifen and Exemestane Adjuvant Multinational (TEAM) clinical trial cohort that low IKKα expression is associated with increased risk of recurrence on sequential tamoxifen/exemestane therapy, suggesting that the role of IKKα in hormone therapy resistance may change depending on the mechanism of action of the therapy the patient receives [83].

In gastric cancer, Helicobacter pylori -mediated NF-κB activation is thought to occur via an IKKα-linked pathway that is independent of the non-canonical NF-κB pathway, but involves both IKKα and NIK to up-regulate inflammatory infiltrate and promote tumourigenesis [84 ]. Studies of IKKα independent of the non-canonical NF-κB pathway in colorectal cancer and cutaneous squamous cell carcinoma have centred on a truncated form of IKKα (p45 IKKα) that is constitutively active and specifically resides in the nucleus [55, 56 ]. Bennett et al. observed that nuclear IKKα in breast cancer has a stronger predictive power than cytoplasmic IKKα, and proposed that this could be due to detection of the truncated activated form of p45 IKKα as the antibody employed was unable to distinguish between full length IKKα and the truncated p45 IKKα form [71 ]. Other studies of IKKα signalling independent of the non-canonical NF-κB pathway in colorectal cancer provide additional evidence that IKKα binds to Notch-dependent gene promoters to upregulate them and release chromatin-bound SMRT, which can be restored by inhibition IKKα and results in colorectal cancer xenografts shrinking in size [56 ]. It has been reported that the truncated p45 IKKα, forms a complex with full length IKKα and NEMO and is responsible for regulating phosphorylation of SMRT and histone H3 in an NF-κB-independent fashion. In addition, p45 IKKα may be phosphorylated in a TAK1 -dependent but NF-κB-independent manner in BRAFV600^{E} mutant colorectal tumours [56 ], so supporting a role for nuclear IKKα independent of non-canonical NF-κB signalling.

The nuclear role of IKKα is consistently reported as being independent of NF-κB, by activating alternative pathways such as NOTCH [85 ]. This has been observed in breast cancer, skin cancer and osteosarcoma [86 ]. In liver cancer Hepatitis B virus X protein down-regulates maspin expression via nuclear IKKα resulting in chemoresistance, suggesting that targeting IKKα could re-sensitise HCC tumours to chemotherapy [87 ]. In transgenic adenocarcinoma of the mouse prostate (TRAMP) models of prostate cancer IKKα can translocate to the nucleus to promote metastasis and development of castrate resistant disease in a maspin dependent manner, which is accompanied by a local inflammatory response [88 ]. Similar to breast cancer, in prostate cancer nuclear IKKα appears to provide a mechanism for hormone resistance as IKKα is associated with development of castrate resistant prostate cancer [53 ] and deletion of BAG3 which is required for IKKα nuclear translocation delays development of castrate resistant disease [89].

IKKα Association with Hallmarks of Cancer in Human Tumours

The NF-κB pathways regulate the transcription of a wide range of genes involved in the inflammation, proliferation and apoptosis. Many of these processes are hallmarks of cancer [46, 47 ] and NF-κB has been hypothesised to be a link between inflammation and tumourigenesis. Whether IKKα functions as a member of the non-canonical NF-κB pathway or in its NF-κB-independent roles, it is clear that it is involved with multiple hallmarks of cancer including key roles in innate and adaptive immune responses, cell survival, cell death and inflammation [90, 91 ]. The non-canonical NF-κB pathway has key roles in regulating processes including production of lymphoid organs (responsible for B and T lymphocyte production), B-cell development and survival, dendritic cell function and bone metabolism [92 ] and has been reported to promote development and progression of cancers via promotion of inflammatory infiltrate. Mouse model studies have demonstrated that mice with a dominant-negative, catalytically-inactive IKKα, have reduced adenoma formation, smaller colorectal tumours with a lower proliferation index when treated with a carcinogen and this was associated with increased recruitment of macrophages and other immune cell types [93 ]. In skin cancer studies, IKKα has been demonstrated to induce inflammation-related genes [94 ]. In an additional study using a model of peritoneal metastasis in immune-competent mice, intraperitoneal injection with IκBa suppressed colon cells induced an M1 -like macrophage phenotype, with reduced liver and peritoneal metastases in vivo. This was associated with increased intra-tumoural activated CD4 ⁺ and CD8 ⁺ T cells and reduced angiogenesis [93 ], demonstrating that NF-κB pathways work with local inflammatory infiltrate to promote colorectal cancer progression. In renal cancer the inflammatory effects of the NF-κB pathway have mainly been attributed to the canonical p65 /p50 subunits in conjunction with STAT3 . However, NIK and RelB have previously been shown to be crucial for B-cell development [2 ], suggesting that the non-canonical NF-κB pathway also plays a role and that RelB can modulate local inflammatory infiltrate in renal cell carcinoma. IKKα is also associated with promoting expression of pro-inflammatory cytokines such as IL-8 in prostate cancer [95].

Kong et al. suggests that IKKα can be phosphorylated via deleted in breast cancer 1 (DBC1 ) to regulate B cell activation via RelB activity and causing increased cell proliferation in mice [96 ]. In addition, polymerase chain reaction (PCR) array-based gene transcriptional profiling experiments demonstrated that reducing cellular IKKα expression had a significant impact on increased expression of genes associated with induction of apoptosis, in particular BAK1 and BBC3, providing evidence that IKKα is involved regulating both cell proliferation and apoptosis in ER positive breast cancer. Dan et al. demonstrates that IKKα via mTORC can induce cell proliferation in cervical, lung, prostate and pancreatic cell lines [79 ] and in basal cell carcinoma IKKα is associated with proliferation and EMT [94 ]. Studies in vitro also demonstrate that ovarian cancer epithelial cell proliferation, migration and an invasive phenotype of the cancer were promoted via up-regulation of IKKα. In addition, NIK levels have been associated with regulating both cell proliferation and apoptosis in colorectal cancer, demonstrating that the non-canonical NF-κB pathway is involved in cell viability and tumour growth [97].

### IKKα in Haematological Malignancies

Aberrant NF-κB signalling and associated gene transcription that modulate cellular processes involved in the initiation, maintenance and progression of human malignancies are also common to haematological cells and cancers. In this regard, many B-cell leukaemias and lymphomas display abnormal NF-κB activation, implicating this family of transcription factors in these diseases and suggesting regulation of these proteins may represent promising therapeutic targets. In addition, it is now appreciated that conventional cytotoxic agents can increase NF-κB activation, contributing to the development of drug resistance via a number of distinct mechanisms. Therefore, inhibitors that target NIK-IKKα-mediated signalling may prove clinically useful as single agents and also to re-sensitise patients to chemotherapeutic drugs. Given the frequency of genetic mutations in the non-canonical NF-κB pathway and its critical role in tumour microenvironmental signalling, IKKα represents an attractive anti-cancer target.

Chronic lymphocytic leukaemia (CLL) is the commonest leukaemia in Europe and North America. It is characterised by the accumulation of mature-looking CD5 */CD19 ⁺ B lymphocytes in the peripheral blood, bone marrow, and lymphoid tissues [105 ]. NF-κB is constitutively activated in many CLL patients and this is associated with more aggressive disease [106, 107 ]. A number of recurrent genetic mutations in NF-κB-associated genes have been described in CLL. The most common of these is an inactivating mutation in NFκBIE that encodes IκBε , a negative NF-κB regulator. These NFκBIE aberrations are found in approximately 7 % of CLL cases and predominantly occur in poor-prognostic subgroups. This may be causal as mutations in NFκBIE result in increased nuclear translocation of ReIA [108 ]. NOTCH1 mutations occur at an even higher frequency in CLL (~11 %). These activating mutations are associated with poor response to chemotherapy [109 ] and this may be caused by NOTCHI-mediated NF-κB pathway activation [110-112 ]. BIRC3 mutations are found in a smaller proportion of CLL patients (~4 %) but they impact upon the non-canonical NF-κB pathway due to the premature truncation of the BIRC3 -encoded protein product, cIAP2, resulting in the loss of its E3 ubiquitin ligase activity that is essential for NIK proteasomal degradation. As a consequence, NIK levels increase leading to the phosphorylation of IKKα, NF-κB2, the processing of p100 to p52 and the constitutive activation of non-canonical NF-κB signalling [113 ]. Importantly, BIRC3 mutations are associated with loss of sensitivity to chemotherapy and poor prognosis [114].

In addition to the genetic causes of NF-κB dysregulation in CLL, it is now understood that the lymph node microenvironment plays a critical role in modulating the natural pathology of this disease. Signalling via the B-cell receptor (BCR), toll-like receptors (TLR) and CD40, as well as engagement of the BAFF and a proliferation-inducing ligand (APRIL) receptors TACI, BAFF-R and BCMA, create a pro-survival, pro-proliferative niche mediated by NF-κB activation [116, 117 ]. The importance of this microenvironment is perhaps best exemplified by the remarkable clinical effects of the Bruton's tyrosine kinase inhibitor ibrutinib. Treatment with this drug results in a marked tissue redistribution effect with leukaemia cells being excluded from the lymphoid tissues [118 ]. The partitioning of the tumour away from the sites of increased NF-κB signalling results in durable remissions, an effect that is reversed on drug withdrawal.

Diffuse large B-cell lymphomas (DLBCL) are the most common types of non-Hodgkin lymphoma. They are divided into three molecular sub-types: ABC (activated B-cell), GCB (germinal centre B-cell) and PMBL (primary mediastinal B-cell lymphoma). Initial evidence for the role of the canonical NF-κB pathway in DLBCL came from gene expression profiling studies, which showed enrichment for NF-κB target genes in the ABC sub-type. This group has the worst prognosis implicating NF-κB as a modulator of clinical outcome in DLBCL [123 ]. Constitutive NF-κB activation in the ABC sub-type can result from mutations in components of the BCR signalling cascade, which results in chronic BCR activation. These mutations often occur in the immunoreceptor tyrosine-based motif (ITAM) but also in the coiled-coil domain of the CARD11 /CARMA1 gene [124 ]. Finally, MYD88 gene mutations are found in approximately 30 % of the ABC sub-type resulting in spontaneous activation of the downstream IRAK complex and NF-κB activation [125 ]. The non-canonical NF-κB pathway is also aberrantly dysregulated in 10-15 % of DLBCL cases due to TRAF2 and TRAF3 mutations [126 ] and consequently identifies a sub-population of tumours that may be targetable via IKKα.

Multiple myeloma (MM) is an incurable plasma cell malignancy accounting for approximately 13 % of all haematological cancers. Disease progression involves clonal expansion of transformed plasma cells in the bone marrow. Overall, genetic abnormalities leading to constitutive NF-κB activity have been found in approximately 20 % of MM patients and 40 % of MM cell lines [127-129 ]. Most of the genetic abnormalities relating to NF-κB dysregulation in MM involve the non-canonical NF-κB pathway including aberrant expression of NIK, CD40, TRAF2, TRAF3, transmembrane activator and CAML interactor (TACI) and cIAP1/2 [127, 138 ]. In these studies, the majority of MM cases possessed overexpression of the positive NF-κB regulators NIK, TACI and CD40, or reduced or silenced activity of the negative NF-κB regulators TRAF2, TRAF3 and clAP1/2 . All of these phenotypes contribute to increased NF-κB signalling, with a preference towards non-canonical NF-κB signalling [128, 129 ]. In addition, other less common genetic abnormalities that also lead to constitutive NF-κB signalling in MM have been identified. These included high expression of the NFκB1 gene (p105 ) and abnormalities within the NFκB2 gene (p100 ), which results in increased canonical and non-canonical NF-κB signalling, respectively [127-129].

Although genetic abnormalities can explain some of the high NF-κB activity in MM, it is likely that a substantial portion of the NF-κB signalling in this disease arises as a consequence of interactions within the bone marrow microenvironment [129 ]. One such mechanism for NF-κB activation is via CD40 -CD40 L interactions [130, 131 ]. CD40 is a cell surface marker not usually expressed on normal plasma cells but has been shown to be increased in the early stages of MM [132 ]. Furthermore, blocking the interaction of CD40 with CD40 L decreases NF-κB activation [127 ]. This results in the inhibition of IL-6 and vascular endothelial growth factor (VEGF) secretion, which in turn leads to growth arrest and cell death of MM cells [133 ]. Furthermore, the bone marrow stromal cells (BMSC) found in the MM tumour microenvironment have also been found to express high levels of NF-κB activation that helps to support the proliferation, survival and drug resistance of malignant plasma cells within the bone marrow niche [134 ]. Adherence of MM cells to BMSCs induces NF-κB-dependent cytokine transcription and secretion of TNFα, IL-6, VEGF, RANKL and BAFF, to promote MM cell survival and growth through MM cell NF-κB activation [135, 136].

### REFERENCES

[57 ] Duran C L, Lee D W, Jung J U, Ravi S, Pogue C B, Toussaint L G, Bayless K J, Sitcheran R. NIK regulates MT1 -MMP activity and promotes glioma cell invasion independently of the canonical NF-κB pathway. Oncogenesis. 2016 Jun. 6; 5 (6 ):e231.
[58 ] Cherry E, Lee D, Jung J, Sitcheran R_{g} Non-canonical nf-kb signaling drives the aggressive invasiveness of glioblastoma Neuro-oncology 2014 16 (Suppl 5 ), v2.
[59 ] Lessard L, Begin L R, Gleave M E, Mes-Masson A M, Saad F. Nuclear localisation of nuclear factor-kappaB transcription factors in prostate cancer: an immunohistochemical study. Br J Cancer. 2005Oct. 31; 93 (9 ):1019-23.
[60 ] Lessard L, Saad F, Le Page C, Diallo J S, Peant B, Delvoye N, Mes-Masson A M. N F-kappaB2 processing and p52 nuclear accumulation after androgenic stimulation of LNCaP prostate cancer cells. Cell Signal. 2007 May; 19 (5 ):1093-100.
[61 ] Doppler H, Liou G Y, Storz P.Downregulation of TRAF2 mediates NIK-induced pancreatic cancer cell proliferation and tumorigenicity. PLoS One. 2013;8 (I):e53676.
[61 ] Thu Y M, Richmond A. NF-κB inducing kinase: a key regulator in the immune system and in cancer. Cytokine Growth Factor Rev. 2010 August; 21 (4 ):213-26.
[62 ] Nishina T, Yamaguchi N, Gohda J, Semba K, Inoue J. NIK is involved in constitutive activation of the alternative N F-kappaB pathway and proliferation of pancreatic cancer cells. Biochem Biophys Res Commun. 2009Oct. 9; 388 (I):96-101.
[63 ] Chandler N M, Canete J J, Callery M P. Increased expression of N F-kappa B subunits in human pancreatic cancer cells. J Surg Res. 2004 May 1;118 (I):9-14
[64 ] Ishikawa H, Akedo I, Suzuki T, Narahara H, Otani T. Adverse effects of sulindac used for prevention of colorectal cancer. J Natl Cancer Inst. 1997 Sep. 17; 89 (18 ):1381.
[65 ] Lua J, Qayyum, T., Edwards, J. and Roseweir, A. K. The prognostic role of the non-canonical N F-kappa B pathway in renal cell carcinoma patients. Urologia Internationalis. 2018 : accepted.
[66 ] Jamieson S, Fuller P J. Characterization of the inhibitor of kappaB kinase (IKK) complex in granulosa cell tumors of the ovary and granulosa cell tumor-derived cell lines. Horm Cancer. 2013October: 4:277-92.
[67 ] Cildir G, Low K C, Tergaonkar V. Noncanonical N F-kappa B Signaling in Health and Disease. Trends Mol Med. 2016 May: 22:414-29.
[68 ] Qin H, Zhou J, Zhou P, Xu J, Tang Z, Ma H, Guo F. Prognostic significance of RelB overexpression in non-small cell lung cancer patients. Thorac Cancer. 2016 July;7 (4 ):415-21
[69 ] Shen M, Duan X, Zhou P, Zhou W, Wu X, Xu S, Chen Y, Tao Z. Lymphotoxin p receptor activation promotes bladder cancer in a nuclear factor-K B-dependent manner. Mol Med Rep. 2015 February;11 (2 ):783-90.
[70 ] Sovak M A, Bellas R E, Kim D W, Zanieski G J, Rogers A E, Traish A M, Sonenshein G E. Aberrant nuclear factor-kappaB/Rel expression and the pathogenesis of breast cancer. J Clin Invest. 1997 Dec. 15; 100 (12 ):2952-60.
[71 ] Bennett L, Quinn J, McCall P, Mallon E A, Horgan P G, McMillan D C, Paul A, Edwards J. High IKKα expression is associated with reduced time to recurrence and cancer specific survival in oestrogen receptor (E R)-positive breast cancer. Int J Cancer. 2017 Apr. 1; 140 (7 ):1633-1644.
[72 ] Cao Y, Bonizzi G, Seagroves T N, Greten F R, Johnson R, Schmidt E V, Karin M. IKKα lpha provides an essential link between RANK signaling and cyclin D1 expression during mammary gland development. Cell. 2001 Dec. 14; 107 (6 ):763-75.
[73 ] Yang Z, Wang X L, Bai R, Liu W Y, Li X, Liu M, Tang H. miR-23 a promotes IKKα expression but suppresses ST7 L expression to contribute to the malignancy of epithelial ovarian cancer cells. Br J Cancer. 2016 Sep. 6; 115 (6 ):731-40.
[74 ] Connelly L, Robinson-Benion C, Chont M, Saint-Jean L, Li H, Polosukhin V V, Blackwell T S, Yull F E. A transgenic model reveals important roles for the N F-kappa B alternative pathway (p100 /p52 ) in mammary development and links to tumorigenesis. J Biol Chem. 2007 Mar. 30; 282 (13 ):10028-35.
[75 ] Karin M, Bonnizi G, Cao Y. N F-kB: a factor that provides a link between stress, inflammation and cancer. European Journal of Cancer. 2002 November: 38 :S116.
[76 ] Karin M, Cao Y X, Greten F R, Li Z W. N F-kappa B in cancer: From innocent bystander to major culprit. Nature Reviews Cancer. 2002 April: 2:301-10.
[77 ] Karin M, Lin A. N F-kappa B at the crossroads of life and death. Nat Immunol. 2002 March: 3:221-7.
[78 ] Cogswell P C, Guttridge D C, Funkhouser W K, Baldwin A S, Jr. Selective activation of N F-kappa B subunits in human breast cancer: potential roles for N F-kappa B2 /p52 and for Bcl-3. Oncogene. 2000 Feb. 24:19:1123-31.
[79 ] Dan H C, Antonia R J, Baldwin A S. PI3 K/Akt promotes feedforward mTORC2 activation through IKK alpha. Oncotarget. 2016 Apr. 19: 7:21064-75.
[80 ] Espinosa L, Margalef P, Bigas A. Non-conventional functions for N F-kappa B members: the dark side of N F-kappa B. Oncogene. 2015 Apr. 30: 34:2279-87.
[81 ] Rizzo P, Miao H, D'Souza G, Osipo C, Song L L, Yun J, Zhao H, Mascarenhas J, Wyatt D, Antico G, Hao L, Yao K, Rajan P, Hicks C, Siziopikou K, Selvaggi S, Bashir A, Bhandari D, Marchese A, Lendahl U, Qin J Z, Tonetti D A, Albain K, Nickoloff B J, Miele L.Cross-talk between notch and the estrogen receptor in breast cancer suggests novel therapeutic approaches. Cancer Res. 2008 Jul. 1; 68 (13 ):5226-35.
[82 ] Hao L, Rizzo P, Osipo C, Pannuti A, Wyatt D, Cheung L W, Sonenshein G, Osborne B A, Miele L. Notch-1 activates estrogen receptor-alpha-dependent transcription via IKKα lpha in breast cancer cells. Oncogene. 2010 Jan. 14; 29 (2 ):201-13.
[83 ] Roseweir A K, Bennett L, Dickson A, Cheng K, Quintayo M A, Bayani J, McMillan D C, Horgan P G, van de Velde CJH, Seynaeve C, Hasenburg A, Kieback D G, Markopoulos C, Dirix L Y, Rea D W, Mallon E A, Bartlett JMS, Edwards J.Predictive Biomarkers for Endocrine Therapy: Retrospective Study in Tamoxifen and Exemestane Adjuvant Multinational (TEAM) Trial. J Natl Cancer Inst. 2018 Jun. 1; 110 (6 ):616-627.
[84 ] Merga Y J, O'Hara A, Burkitt M D, Duckworth C A, Probert C S, Campbell B J, Pritchard D M.Importance of the alternative NF-κB activation pathway in inflammation-associated gastrointestinal carcinogenesis. Am J Physiol Gastrointest Liver Physiol. 2016 Jun. 1; 310 (11 ):G1081-90.
[85 ] STRAP Promotes Sternness of Human Colorectal Cancer via Epigenetic Regulation of the NOTCH Pathway. Jin L, Vu T, Yuan G, Datta P K. Cancer Res. 2017Oct. 15; 77 (20 ):5464-5478.
[86 ] Leopizzi M, Cocchiola R, Milanetti E, Raimondo D, Politi L, Giordano C, Scandurra R, Scotto d'Abusco A.IKKα inibition by a glucosamine derivative enhances Maspin expression in osteosarcoma cell line. Chem Biol Interact. 2017 Jan. 25; 262:19-28.
[87 ] Cheng KKW, Bennett L, Edwards J. Identification of a novel biomarker of IKK alpha-dependent N F-kappa B signalling in oestrogen receptor (E R)-positive breast cancer. Scot Med J. 2016 November: 61 :Np55.
[88 ] Luo J L, Tan W, Ricono J M, Korchynskyi O, Zhang M, Gonias S L, Cheresh D A, Karin M.Nuclear cytokine-activated IKKα lpha controls prostate cancer metastasis by repressing Maspin. Nature. 2007 Apr. 5; 446 (7136 ):690-4.
[89 ] Ammirante M, De Laurenzi V, Graziano V, Turco M C, Rosati A. BAG3 is required for IKKα nuclear translocation and emergence of castration resistant prostate cancer. Cell Death Dis. 2011 Mar. 31; 2 :e139.
[90 ] Rizel L, Safieh C, Shalev S A, Mezer E, Jabaly-Habib H, Ben-Neriah Z, Chervinsky E, Briscoe D, Ben-Yosef T.Novel mutations of MYO7 A and USH1 G in Israeli Arab families with Usher syndrome type 1 . Mol Vis. 2011; 17:3548-55.
[91 ] Ben-Neriah Y, Karin M. Inflammation meets cancer, with N F-kappa B as the matchmaker. Nat Immunol. 2011 August: 12:715-23.
[92 ] Karin M, Greten F R. N F kappa B: Linking inflammation and immunity to cancer development and progression. Nature Reviews Immunology. 2005October: 5:749-59
[93 ] Sepulveda A, Soriano H, Espino A.Gastrointestinal tract involvement in Klippel-Trenaunay syndrome. Lancet Gastroenterol Hepatol. 2018 July;3 (7 ):518.
[94 ] Jia J, Shi Y, Yan B, Xiao D, Lai W, Pan Y, Jiang Y, Chen L, Mao C, Zhou J, Xi S, Cao Y, Liu S, Tao Y. LGR5 expression is controled by IKKα in basal cell carcinoma through activating STAT3 signaling pathway. Oncotarget. 2016 May 10;7 (19 ):27280-94.
[95 ] Manna S, Singha B, Phyo S A, Gatla H R, Chang T P, Sanacora S, Ramaswami S, Vancurova I.Proteasome inhibition by bortezomib increases IL-8 expression in androgen-independent prostate cancer cells: the role of IKKα. J Immunol. 2013 Sep. 1; 191 (5 ):2837-46.
[96 ] Kong S, Dong H, Song J, Thiruppathi M, Prabhakar B S, Qiu Q, Lin Z, Chini E, Zhang B, Fang D.Deleted in Breast Cancer 1 Suppresses B Cell Activation through RelB and Is Regulated by IKKα Phosphorylation. J Immunol. 2015Oct. 15; 195 (8 ):3685-93.
[97 ] Qu L L, He L, Zhao X, Xu W. Downregulation of miR-518 a-3 p activates the NIK-dependent N F-kappa B pathway in colorectal cancer. Int J Mol Med. 2015 May: 35:1266-72.
[98 ] Frelin C, Imbert V, Griessinger E, Peyron A C, Rochet N, Philip P, Dageville C, Sirvent A, Hummelsberger M, Berard E, Dreano M, Sirvent N, Peyron J F. Targeting N F-kappaB activation via pharmacologic inhibition of IKK2 -induced apoptosis of human acute myeloid leukemia cells. Blood. 2005 Jan. 15; 105 (2 ):804-11
[99 ] Hehner S P, Hofmann T G, Dr6 ge W, Schmitz M L. The antiinflammatory sesquiterpene lactone parthenolide inhibits N F-kappa B by targeting the I kappa B kinase complex. J Immunol. 1999 Nov. 15; 163 (10 ):5617-23
[100 ] Hideshima T, Chauhan D, Kiziltepe T, Ikeda H, Okawa Y, Podar K, Raje N, Protopopov A, Munshi N C, Richardson P G, Carrasco R D, Anderson K C. Biologic sequelae of I{kappa}B kinase (IKK) inhibition in multiple myeloma: therapeutic implications. Blood. 2009 May 21;113 (21 ):5228-36.
[101 ] Coope H J, Atkinson P G, Huhse B, Belich M, Janzen J, Holman M J, Klaus G G, Johnston L H, Ley S C. CD40 regulates the processing of N F-kappaB2 p100 to p52. 2002Oct. 15; 21 (20 ):5375-85.
[102 ] Kayagaki N, Yan M, Seshasayee D, Wang H, Lee W, French D M, Grewal I S, Cochran A G, Gordon N C, Yin J, Starovasnik M A, Dixit V M. BAFF/BLyS receptor 3 binds the B cell survival factor BAFF ligand through a discrete surface loop and promotes processing of N F-kappaB2. 2002October;17 (4 ):515-24
[103 ] Novack D V, Yin L, Hagen-Stapleton A, Schreiber R D, Goeddel D V, Ross F P, Teitelbaum S L.The IkappaB function of N F-kappaB2 p100 controls stimulated osteoclastogenesis. 2003 Sep. 1; 198 (5 ):771-81
[104 ] Sun S.C. The non-canonical NF-κB pathway. Immunol. Rev. 2012; 246:125-140.
[105 ] Scarfò L, Ferreri A J, Ghia P. Chronic lymphocytic leukaemia. Crit Rev Oncol Hematol. 2016; 104:169-82.
[106 ] Cuni S., Pérez-Aciego P., Pérez-Chac6 n G., Vargas J A., Sánchez A., Martín-Saavedra F.M., Ballester S., García-Marco J., Jordá J., Durántez A. A sustained activation of PI3 K/NF-κB pathway is critical for the survival of chronic lymphocytic leukemia B cells. Leukemia. 2004; 18:1391-1400.
[107 ] Hewamana S, Alghazal S, Lin T T, Clement M, Jenkins C, Guzman M L, Jordan C T, Neelakantan S, Crooks P A, Burnett A K, Pratt G, Fegan C, Rowntree C, Brennan P, Pepper C. The N F-kappaB subunit Rel A is associated with in vitro survival and clinical disease progression in chronic lymphocytic leukemia and represents a promising therapeutic target. Blood. 2008 May 1;111 (9 ):4681-9.
[108 ] Mansouri L, Sutton L A, Ljungström V, Bondza S, Amgárden L, Bhoi S, Larsson J, Cortese D, Kalushkova A, Plevova K, Young E, Gunnarsson R, Falk-Sörqvist E, Lönn P, Muggen A F, Yan X J, Sander B, Enblad G, Smedby K E, Juliusson G, Belessi C, Rung J, Chiorazzi N, Strefford J C, Langerak A W, Pospisilova S, Davi F, Hellstr6 m M, Jemberg-Wiklund H, Ghia P, Söderberg O, Stamatopoulos K, Nilsson M, Rosenquist R.Functional loss of IκBε leads to N F—K B deregulation in aggressive chronic lymphocytic leukemia. J Exp Med. 2015 Jun. 1; 212 (6 ):833-43
[109 ] Fabbri G, Rasi S, Rossi D, Trlfonov V, Khiabanian H, Ma J, Grunn A, Fangazio M, Capello D, Monti S, Cresta S, Gargiulo E, Forconi F, Guarini A, Arcaini L, Paulli M, Laurenti L, Larocca L M, Marasca R, Gattei V, Oscier D, Bertoni F, Mullighan C G, Foá R, Pasqualucci L, Rabadan R, Dalla-Favera R, Galdano G. Analysis of the chronic lymphocytic leukemia coding genome: role of NOTCHI mutational activation. J Exp Med. 2011 Jul. 4; 208 (7 ):1389-401.
[110 ] Rosati E, Sabatini R, Rampino G, Tabillo A, Di lanni M, Fettucciarl K, Bartoll A, Coaccioli S, Screpanti I, Marconi P. Constitutively activated Notch signaling is involved in survival and apoptosis resistance of B-CLL cells. Blood. 2009 . Jan. 22; 113 (4 ):856-65
[111 ] Baliakas P, Hadzidimitriou A, Sutton L A, Rossi D, Minga E, Villamor N, Larrayoz M, Kminkova J, Agathangelidis A, Davis Z, Tausch E, Stalika E, Kantorova B, Mansouri L, Scarft L, Cortese D, Navrkalova V, Rose-Zerilli M J, Smedby K E, Juliusson G, Anagnostopoulos A, Makris A M, Navarro A, Delgado J, Oscier D, Belessi C, Stilgenbauer S, Ghia P, Pospisilova S, Gaidano G, Campo E, Strefford J C, Stamatopoulos K, Rosenquist R. Recurrent mutations refine prognosis in chronic lymphocytic leukemia. European Research Initiative on CLL (ERIC). Leukemia. 2015 February;29 (2 ):329-36
[112 ] Chiaretti S, Marinelli M, Del Giudice I, Bonina S, Piciocchi A, Messina M, Vignetti M, Rossi D, Di Maio V, Mauro F R, Guarini A, Gaidano G, Fob R. NOTCHI, SF3B1, BIRC3 and TP53 mutations in patients with chronic lymphocytic leukemia undergoing first-line treatment: correlation with biological parameters and response to treatment. Leuk Lymphoma. 2014 December;55 (12 ):2785-92
[113 ] Dejardin E. Biochem Pharmacol. The alternative N F-kappaB pathway from biochemistry to biology: pitfalls and promises for future drug development. 2006; 72 (9 ):1161-79.
[114 ] Rossi D1, Rasi S, Fabbri G, Spina V, Fangazio M, Forconi F, Marasca R, Laurenti L, Bruscaggin A, Cerr M, Monti S, Cresta S, FamA R, De Paoli L, Bulian P, Gattei V, Guarini A, Deaglio S, Capello D, Rabadan R, Pasqualucci L, Dalla-Favera R, Foá R, Gaidano G. Mutations of NOTCHI are an independent predictor of survival in chronic lymphocytic leukemia. Blood. 2012 Jan. 12; 119 (2 ):521-9.
[115 ] Puente X S, Pinyol M, Quesada V, Conde L, Ordóñez G R, Villamor N, Escaramis G, Jares P, Beà S, González-Diaz M, Bassaganyas L, Baumann T, Juan M, Lòpez-Guerra M, Colomer D, Tubío J M, Lòpez C, Navarro A, Tornador C, Aymerich M, Rozman M, Hernández J M, Puente D A, Freije J M, Velasco G, Gutiérrez-Fernández A, Costa D, Carrió A, Guijarro S, Enjuanes A, Hernández L, Yague J, Nicolás P, Romeo-Casabona C M, Himmelbauer H, Castillo E, Dohm J C, de Sanjosé S, Piris M A, de Alava E, San Miguel J, Royo R, Gelpi J L, Torrents D, Orozco M, Pisano D G, Valencia A, Guig6 R, Bayds M, Heath S, Gut M, Klatt P, Marshall J, Raine K, Stebbings L A, Futreal P A, Stratton M R, Campbell P J, Gut I, Lòpez-Guillermo A, Estivill X, Montserrat E, Lòpez-Otin C, Campo E. Whole-genome sequencing identifies recurrent mutations in chronic lymphocytic leukaemia. 2011 Jun. 5; 475 (7354 ):101-5.
[116 ] Herishanu Y, Pérez-Galán P, Liu D, Blancotto A, Pittaluga S, Vire B, Gibellini F, Njuguna N, Lee E, Stennett L, Raghavachari N, Liu P, McCoy J P, Raffeld M, Stetler-Stevenson M, Yuan C, Sherry R, Arthur D C, Maric I, White T. Marti G E, Munson P, Wilson W H, Wiestner A.The lymph node microenvironment promotes B-cell receptor signaling, N F-kappaB activation, and tumor proliferation in chronic lymphocytic leukemia. Blood. 2011 Jan. 13; 117 (2 ):563-74.
[117 ] Rosén A., Murray F., Evaldsson C., Rosenquist R. Antigens in chronic lymphocytic leukemla-Implications for cell origin and leukemogenesis. Semin. Cancer Biol. 2010; 20:400 -409.
[118 ] Wodarz D, Garg N, Komarova N L, Benjamini O, Keating M J, Wierda W G, KantarJian H, James D, O'Brien S, Burger J A. Kinetics of CLL cells in tissues and blood during therapy with the BTK inhibitor ibrutinib. Blood. 2014 Jun. 26; 123 (26 ):4132-5.
[119 ] Lucas P C, Kuffa P, Gu S, Kohrt D, Kim D S, Siu K, Jin X, Swenson J, McAllister-Lucas LMA dual role for the API2 moiety in API2 -MALT1 -dependent N F-kappaB activation: heterotypic oligomerization and TRAF2 recruitment. 2007 Aug. 16; 26 (38 ):5643-54.
[120 ] Rosebeck S. Madden L, Jin X, Gu S, Apel I J, Appert A, Hamoudi R A, Noels H, Sagaert X, Van Loo P, Baens M, Du M O, Lucas P C, McAllister-Lucas L M. Cleavage of NIK by the API2 -MALT1 fusion oncoprotein leads to noncanonical N F-kappaB activation. 2011 Jan. 28; 331 (6016 ):468-72.
[121 ] Spina V, Rossi D. NF-κB deregulation in splenic marginal zone lymphoma. Semin. Cancer Biol. 2016; 39:61-67.
[122 ] Thu Y M, Richmond A. NF-κB inducing kinase: a key regulator in the immune system and in cancer. Cytokine Growth Factor Rev. 2010; 21 (4 ):213-26.
[123 ] Davis R E, Brown K D, Siebenlist U, Staudt L M. Constitutive nuclear factor κB activity is required for survival of activated B cell-like diffuse large B cell lymphoma cells. J. Exp. Med. 2001; 194:1861-1874.
[124 ] Lenz G, Davis R E, Ngo V N, Lam L, George T C, Wright G W, Dave S S, Zhao H, Xu W, Rosenwald A, Ott G, Muller-Hermelink H K, Gascoyne R D, Connors J M, Rimsza L M, Campo E, Jaffe E S, Delabie J, Smeland E B, Fisher R I, Chan W C, Staudt L M. Oncogenic CARD11 mutations in human diffuse large B cell lymphoma. Science. 2008 Mar. 21; 319 (5870 ):1676-9.
[125 ] Ngo V N, Young R M, Schmitz R, Jhavar S, Xiao W, Lim K H, Kohlhammer H, Xu W, Yang Y, Zhao H, Shaffer A L, Romesser P, Wright G, Powell J, Rosenwald A, Muller-Hermelink H K, Ott G, Gascoyne R D, Connors J M, Rimsza L M, Campo E, Jaffe E S, Delabie J, Smeland E B, Fisher R I, Braziel R M, Tubbs R R, Cook J R, Weisenburger D D, Chan W C, Staudt L M.
Oncogenically active MYD88 mutations in human lymphoma. Nature. 2011 Feb. 3; 470 (7332 ):115-9.
[126 ] Zhang B, Calado D P, Wang Z, Frohler S, Kochert K, Qian Y, Koralov S B, Schmidt-Supprian M, Sasaki Y, Unitt C, Rodig S, Chen W, Dalla-Favera R, Alt F W, Pasqualucci L, Rajewsky K. An oncogenic role for alternative NF-κB signaling in DLBCL revealed upon deregulated BCL6 expression. Cell Rep. 2015 May 5;11 (5 ):715-26 .
[127 ] Annunziata C M, Davis R E, Demchenko Y, Bellamy W, Gabrea A, Zhan F, Lenz G, Hanamura I, Wright G, Xiao W, Dave S, Hurt E M, Tan B, Zhao H, Stephens O, Santra M, Williams D R, Dang L, Barlogie B, Shaughnessy J D Jr, Kuehl W M, Staudt L M. Frequent engagement of the classical and alternative N F-kappaB pathways by diverse genetic abnormalities in multiple myeloma. Cancer Cell. 2007; 12 (2 ):115-30.
[128 ] Keats J J, Fonseca R, Chesi M, Schop R, Baker A, Chng W J, Van Wier S, Tiedemann R, Shi C X, Sebag M, Braggio E, Henry T, Zhu Y X, Fogle H, Price-Troska T, Ahmann G, Mancini C, Brents L A, Kumar S, Greipp P, Dispenzieri A, Bryant B, Mulligan G, Bruhn L, Barrett M, Valdez R, Trent J, Stewart A K, Carpten J, Bergsagel P L. Promiscuous mutations activate the noncanonical N F-kappaB pathway in multiple myeloma. Cancer Cell. 2007; 12 (2 ):131-44.
[129 ] Demchenko Y N, Glebov O K, Zingone A, Keats J J, Bergsagel P L, Kuehl W M. Classical and/or alternative N F-kappaB pathway activation in multiple myeloma. Blood. 2010; 115 (17 ):3541-52.
[130 ] Coope HJ1, Atkinson P G, Huhse B, Belich M, Janzen J, Holman M J, Klaus G G, Johnston L H, Ley S C. CD40 regulates the processing of N F-kappaB2 p100 to p52 . EMBO J. 2002; 21 (20 ):5375-85.
[131 ] Hauer J, Püschner S, Ramakrishnan P, Simon U, Bongers M, Federle C, Engelmann H.
TNF receptor (TNFR)-associated factor (TRAF) 3 serves as an inhibitor of TRAF2/5 -mediated activation of the noncanonical N F-kappaB pathway by TRAF-binding TNFRs. Proc Nati Acad Sci USA. 2005; 102 (8 ):2874-9.
[132 ]Pérez-Andres M1, Almeida J, Martin-Ayuso M, De Las Heras N, Moro M J, Martin-Nurlez G, Galende J, Cuello R, Abuin I, Moreno I, Dominguez M, Hernandez J, Mateo G, San Miguel J F, Orfao A. Soluble and membrane levels of molecules involved in the interaction between clonal plasma cells and the immunological microenvironment in multiple myeloma and their association with the characteristics of the disease. Int J Cancer. 2009; 124 (2 ):367-75.
[133 ] Richardson P, Schlossman R, Jagannath S, Alsina M, Desikan R, Blood E, Weller E, Mitsiades C, Hideshima T, Davies F, Doss D, Freeman A, Bosch J, Patin J, Knight R, Zeldis J, Dalton W, Anderson K. Thalidomide for patients with relapsed multiple myeloma after high-dose chemotherapy and stem cell transplantation: results of an open-label multicenter phase 2 study of efficacy, toxicity, and biological activity. Mayo Clin Proc. 2004; 79 (7 ):875-82.
[134 ] McMillin D W, Negri J M, Mitsiades C S. The role of tumour-stromal interactions in modifying drug response: challenges and opportunities. Nat Rev Drug Discov. 2013; 12 (3 ):217 -28.
[135 ] Chauhan D, Uchlyama H, Akbarali Y, Urashima M, Yamamoto K, Libermann T A, Anderson K C. Multiple myeloma cell adhesion-induced interleukin-6 expression in bone marrow stromal cells involves activation of N F-kappa B. Blood. 1996; 87 (3 ):1104-12 .
[136 ] Bommert K, Bargou R C, Stühmer T. Signalling and survival pathways in multiple myeloma. Eur J Cancer. 2006; 42 (11 ):1574-80 .

Thus, according to a further aspect of the present invention, there is provided a method of inhibiting IKKα activity, in vitro or in vivo, said method comprising contacting a cell with an effective amount of a compound or a pharmaceutically acceptable salt, hydrate or solvate thereof as defined herein.

According to a further aspect of the present invention, there is provided a method of treating a disease or disorder in which IKKα activity is implicated in a patient in need of such treatment, said method comprising administering to said patient a therapeutically effective amount of a compound or a pharmaceutically acceptable salt, hydrate or solvate thereof as defined herein, or a pharmaceutical composition as defined herein.

According to a further aspect of the present invention, there is provided a method of treating a proliferative disorder in a patient in need of such treatment, said method comprising administering to said patient a therapeutically effective amount of a compound or a pharmaceutically acceptable salt, hydrate or solvate thereof as defined herein, or a pharmaceutical composition as defined herein.

According to a further aspect of the present invention, there is provided a method of treating cancer in a patient in need of such treatment, said method comprising administering to said patient a therapeutically effective amount of a compound or a pharmaceutically acceptable salt, hydrate or solvate thereof as defined herein, or a pharmaceutical composition as defined herein.

According to a further aspect of the present invention, there is provided a compound, or a pharmaceutically acceptable salt, hydrate or solvate thereof, or a pharmaceutical composition as defined herein, for use in therapy.

According to a further aspect of the present invention, there is provided a compound, or a pharmaceutically acceptable salt, hydrate or solvate thereof, or a pharmaceutical composition as defined herein, for use as a medicament.

According to a further aspect of the present invention, there is provided a compound or a pharmaceutically acceptable salt, hydrate or solvate thereof as defined herein, or a pharmaceutical composition as defined herein, for use in the treatment of a proliferative disorder.

According to a further aspect of the present invention, there is provided a compound, or a pharmaceutically acceptable salt, hydrate or solvate thereof, or a pharmaceutical composition as defined herein for use in the treatment of cancer. In a particular embodiment, the cancer is human cancer.

According to a further aspect of the present invention, there is provided a compound, or a pharmaceutically acceptable salt, hydrate or solvate thereof, as defined herein for use in the inhibition of IKKα activity.

According to a further aspect of the present invention, there is provided a compound, or a pharmaceutically acceptable salt, hydrate or solvate thereof, as defined herein for use in the treatment of a disease or disorder in which IKKα activity is implicated.

According to a further aspect of the present invention, there is provided the use of a compound, or a pharmaceutically acceptable salt, hydrate or solvate thereof, as defined herein in the manufacture of a medicament for the treatment of a proliferative disorder.

According to a further aspect of the present invention, there is provided the use of a compound, or a pharmaceutically acceptable salt, hydrate or solvate thereof, as defined herein in the manufacture of a medicament for the treatment of cancer.

According to a further aspect of the present invention, there is provided a use of a compound, or a pharmaceutically acceptable salt, hydrate or solvate thereof, as defined herein in the manufacture of a medicament for the inhibition of IKKα activity.

According to a further aspect of the present invention, there is provided a use of a compound, or a pharmaceutically acceptable salt, hydrate or solvate thereof, as defined herein in the manufacture of a medicament for the treatment of a disease or disorder in which IKKα activity is implicated.

According to a further aspect of the present invention, there is provided a process for preparing a compound, or a pharmaceutically acceptable salt, hydrate or solvate thereof, as defined herein.

According to a further aspect of the present invention, there is provided a compound, or a pharmaceutically acceptable salt, hydrate or solvate thereof, obtainable by, or obtained by, or directly obtained by a process of preparing a compound as defined herein.

According to a further aspect of the present invention, there are provided novel intermediates as defined herein which are suitable for use in any one of the synthetic methods set out herein.

The term “proliferative disorder”, “proliferative condition” and “proliferative disease” are used interchangeably herein and pertain to an unwanted or uncontrolled cellular proliferation of excessive or abnormal cells which is undesired, such as, neoplastic or hyperplastic growth, whether in vitro or in vivo.

In the above-outlined aspects of the invention, the proliferative disorder is suitably cancer, and the cancer is suitably a human cancer. In particular, the compounds of the present invention will be useful for the treatment of any cancer in which a mis-match repair inhibition is beneficial. Any suitable cancer may be targeted (e.g. adenoid cystic carcinoma, adrenal gland tumor, amyloidosis, anal cancer, appendix cancer, astrocytoma, ataxia-telangiectasia, Beckwith-Wiedemann Syndrome, bile duct cancer (cholangiocarcinoma), Birt-Hogg-Dube Syndrome, bladder cancer, bone cancer, brain stem glioma, brain tumor, breast cancer, Carney Complex, central nervous system tumors, cervical cancer, colorectal cancer, Cowden Syndrome, craniopharyngioma, desmoplastic infantile ganglioglioma, ependymoma, esophageal cancer, Ewing sarcoma, eye cancer, eyelid cancer, familial adenomatous polyposis, familial GIST, familial malignant melanoma, familial non-VHL clear cell renal cell carcinoma, familial pancreatic cancer, gallbladder cancer, gastrointestinal stromal tumor—GIST, germ cell tumor, gestational trophoblastic disease, head and neck cancer, hereditary breast and ovarian cancer, hereditary diffuse gastric cancer, hereditary leiomyomatosis and renal cell cancer, hereditary mixed polyposis syndrome, hereditary pancreatitis, hereditary papillary renal carcinoma, juvenile polyposis syndrome, kidney cancer, lacrimal gland tumor, laryngeal and hypopharyngeal cancer, leukemia (acute lymphoblastic leukamia (ALL), acute myeloid leukemia (AML), B-cell prolymphocytic leukemia, hairy cell leukemia, chronic lymphocytic leukemia (CLL), chronic myeloid leukemia (CML), chronic T-cell lymphocytic leukemia, eosinophilic leukemia), Li-Fraumeni Syndrome, liver cancer, lung cancer (non-small cell lung cancer, small cell lung cancer), Lymphoma (Hodgkin, non-Hodgkin), Lynch Syndrome, mastocytosis, medulloblastoma, melanoma, meningioma, mesothelioma, multiple endocrine neoplasia Type 1 & 2, multiple myeloma, MUTYH (or MYH)-associated polyposis, myelodysplastic syndromes (MDS), nasal cavity and paranasal sinus Cancer, nasopharyngeal Cancer, neuroblastoma, neuroendocrine tumors (e.g. of the gastrointestinal tract, lung or pancreas), neurofibromatosis Type 1 & 2, nevoid basal cell carcinoma syndrome, oral and oropharyngeal cancer, osteosarcoma, ovarian/fallopian tube/peritoneal cancer, pancreatic cancer, parathyroid cancer, penile cancer, Peutz-Jeghers Syndrome, pheochromocytoma, paraganglioma, pituitary gland tumor, pleuropulmonary blastoma, prostate cancer, retinoblastoma, rhabdomyosarcoma, salivary gland cancer, sarcoma (e.g. Kaposi or soft tissue), skin cancer, small bowel cancer, stomach cancer, testicular cancer, thymoma and thymic carcinoma, thyroid cancer, tuberous sclerosis complex, uterine cancer, vaginal cancer, Von Hippel-Lindau syndrome, vulvar cancer, Waldenstrom's macroglobulinemia, Werner syndrome, Wilms Tumor and xeroderma pigmentosum). Particular cancers of interest include haematological cancers such as lymphomas (including diffuse large B-cell lymphoma (DLBCL), follicular lymphoma (FL), Burkitt lymphoma (BL) and angioimmunoblastic T-cell lymphoma (AITL)), leukaemias (including acute lymphoblastic leukaemia (ALL) and chronic myeloid leukaemia (CML)), multiple myeloma, breast cancer, non-small cell lung cancer (NSCLC), colorectal cancer, endometrial cancer, gastro-oesophageal cancer, neuroendocrine cancers, osteosarcomas, prostate cancer, pancreatic cancer, small intestine cancer, bladder cancer, rectal cancer, cholangiocarcinoma, CNS cancer, thyroid cancer, head and neck cancer, oesophageal cancer, and ovarian cancer.

Particular cancers in which IKKα inhibition is anticipated to be beneficial include advanced prostate cancer, multiple myeloma, pancreatic cancer, colorectal cancer (especially metastatic colorectal cancer) and breast cancer (especially triple negative breast cancer).

Prostate cancer is of particular interest as potential therapeutic target for IKKα inhibitors. Without wishing to be bound by any particular theory, in prostate cancer, the effective targeting of IKKα may enhance androgen deprivation therapy (ADT) chemotherapy responses by concurrently inhibiting androgen-driven and androgen-independent AR (androgen receptor) activity. IKKα inhibition may also abrogate inflammatory microenvironment signalling and eliminate tumour-promoting stimuli from adjacent stroma and infiltrating monocytes. IKKα inhibitors therefore have the potential to alter disease course, restore/prolong sensitivity to AR-targeted therapy and improve survival. Moreover, their use in hormone-sensitive de novo metastatic disease may significantly extend the benefit duration of conventional therapies and reduce the overall incidence of castration-resistant prostate cancer (CRPC). As a consequence, a IKKα inhibitor may find use in clinical scenarios such as:
the last-line therapy in patients with CRPC that have failed standard-of-care treatment
combination therapy with ADT to prevent the emergence of CRPC/prolong sensitivity to ADT
combination therapy in CRPC patients to restore sensitivity to ADT/reduce resistance development to chemotherapy
single-agent therapy to prevent the emergence of CRPC

### Routes of Administration

The compounds of the invention or pharmaceutical compositions comprising these compounds may be administered to a subject by any convenient route of administration, whether systemically, peripherally or topically (i.e., at the site of desired action).

Routes of administration include, but are not limited to, oral (e.g, by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including intratumoral, subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### Combination Therapies

The compounds of the present invention may be administered as a sole therapy or may involve, in addition to a compound of the invention, conventional surgery or radiotherapy or chemotherapy or a targeted agent. Such chemotherapy or targeted agent may include one or more of the following categories:
(i) Antiproliferative/antineoplastic drugs and combinations thereof, as used in medical oncology, such as, but not limited to, alkylating agents (for example cis-platin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5 -fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);
(ii) cytostatic agents such as, but not limited to, antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), steroid hormones, including progestogens (for example megestrol acetate) and corticosteroids (for example dexamethasone, prednisone and prednisolone), aromatase inhibitors (for example as anastrozole, letrozole, vorazole and exemestane) and inhibitors of 5 a-reductase such as finasteride;
(iii) anti-invasion agents such as, but not limited to, c-Src kinase family inhibitors 4 -(6 -chloro-2,3 -methylenedioxyanilino)-7 -[2 -(4 -methylpiperazin-1 -yl)ethoxy]-5 -tetrahydropyran-4 -yloxyquinazoline (AZD0530 ; International Patent Application WO 01/94341 ), N-(2 -chloro-6 -methylphenyl)-2 -{6 -[4 -(2 -hydroxyethyl)piperazin-1 -y1 ]-2 -methylpyrimidin-4 -ylamino}thiazole-5 -carboxamide (dasatinib, BMS-354825 ; J. Med. Chem., 2004, 47, 6658-6661 ), bosutinib (SKI-606 ), and metalloproteinase inhibitors such as marimastat, inhibitors of urokinase plasminogen activator receptor function or antibodies to Heparanase;
(iv) inhibitors of growth factor function such as, but not limited to, growth factor antibodies and growth factor receptor antibodies (for example the anti-erbB2 antibody trastuzumab [Herceptin™], the anti-EGFR antibody panitumumab, the anti-erbB1 antibody cetuximab [Erbitux, C225 ] and any growth factor or growth factor receptor antibodies disclosed by Stern et al. (Critical reviews in oncology/haematology, 2005, Vol. 54, pp11-29 ); such inhibitors also include tyrosine kinase inhibitors, for example inhibitors of the epidermal growth factor family (for example EGFR family tyrosine kinase inhibitors such as N-(3 -chloro-4 -fluorophenyl)-7 -methoxy-6 -(3 -morpholinopropoxy)quinazolin-4 -amine (gefitinib, ZD1839 ), N-(3 -ethynylphenyl)-6,7 -bis(2 -methoxyethoxy)quinazolin-4 -amine (erlotinib, OSI-774 ) and 6 -acrylamido-N-(3 -chloro-4 -fluorophenyl)-7 -(3 -morpholinopropoxy)-quinazolin-4 -amine (Cl 1033 ), erbB2 tyrosine kinase inhibitors such as lapatinib); inhibitors of the hepatocyte growth factor family; inhibitors of the insulin growth factor family; inhibitors of the platelet-derived growth factor family such as imatinib and/or nilotinib (AMN107 ); inhibitors of serine/threonine kinases (for example Ras/Raf signalling inhibitors such as farnesyl transferase inhibitors, for example sorafenib (BAY 43-9006 ), tipifarnib (R115777 ) and lonafarnib (SCH66336 )), inhibitors of cell signalling through MEK and/or AKT kinases, c-kit inhibitors, abl kinase inhibitors, PI3 kinase inhibitors, PIt3 kinase inhibitors, CSF-1 R kinase inhibitors, IGF receptor (insulin-like growth factor) kinase inhibitors; aurora kinase inhibitors and cyclin dependent kinase inhibitors such as CDK2 and/or CDK4 inhibitors;
(v) antiangiogenic agents such as, but not limited to, those which inhibit the effects of vascular endothelial growth factor, [for example the anti-vascular endothelial cell growth factor antibody bevacizumab (Avastin™) and for example, a VEGF receptor tyrosine kinase inhibitor such as vandetanib (ZD6474 ), vatalanib (PTK787 ), sunitinib (SU11248 ), axitinib (AG-013736 ) and pazopanib (GW 786034 ).
(vi) vascular damaging agents such as, but not limited to, Combretastatin A4 and compounds disclosed in International Patent Applications WO 99/02166, WO 00/40529, WO 00/41669, WO 01/92224, WO 02/04434 and WO 02/08213;
(vii) an endothelin receptor antagonist, for example zibotentan (ZD4054 ) or atrasentan;
(viii) antisense therapies, such as, but not limited to, those directed to targets listed above, such as ISIS 2503, an anti-ras antisense;
(ix) immunotherapy approaches, including for example cancer vaccines, antibody, viral (oncolytic viruses) and small molecule or cell therapy approaches to increase the immunogenicity of patient tumour cells and/or facilitate a cell mediated anti-tumour response. Such therapies could include, but are not limited to, OX40 agonists, cGAS-STING agonists, A2 a receptor antagonists, PI3 kinase inhibitors, TLR7/8 agonists, IDO inhibitors, Arginase inhibitors, BTK inhibitors and Bromodomain inhibitors; transduction with microbial vectors of cancer antigens, direct transduction of cancer antigens into antigen presenting cells, treatment with immune cells specific for cancer antigens (e.g. CAR-T), treatment with antibodies, antibody fragments and antibody drug conjugates that enable the immune system to recognise tumour cells.

The compounds of the present invention are anticipated to be particularly useful in combination with androgen deprivation therapies (ADTs) and standard chemotherapy used to treat prostate cancer and, in particular, castrate-resistant prostate cancer (CRPC).

Such conjoint treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

According to this aspect of the invention there is provided a combination for use in the treatment of a cancer (for example a cancer involving a solid tumour) comprising a compound of the invention as defined hereinbefore, or a pharmaceutically acceptable salt or solvate thereof, and an anti-tumour agent.

According to this aspect of the invention there is provided a combination for use in the treatment of a proliferative condition, such as cancer (for example a cancer involving a solid tumour), comprising a compound of the invention as defined hereinbefore, or a pharmaceutically acceptable salt or solvate thereof, and any one of the anti-tumour agents listed herein above.

In a further aspect of the invention there is provided a compound of the invention or a pharmaceutically acceptable salt or solvate thereof, for use in the treatment of cancer in combination with another anti-tumour agent, optionally selected from one listed herein above.

In a further aspect of the invention there is provided a compound of the invention or a pharmaceutically acceptable salt or solvate thereof, for use in the treatment of cancer in combination with a tyrosine kinase inhibitor, optionally selected from one listed herein above.

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination.

According to a further aspect of the invention there is provided a pharmaceutical composition which comprises a compound of the invention, or a pharmaceutically acceptable salt or solvate thereof, in combination with an anti-tumour agent (optionally selected from one listed herein above), in association with a pharmaceutically acceptable diluent or carrier.

### EXAMPLES

### Inhibitor Design and Structure-Activity Relationship

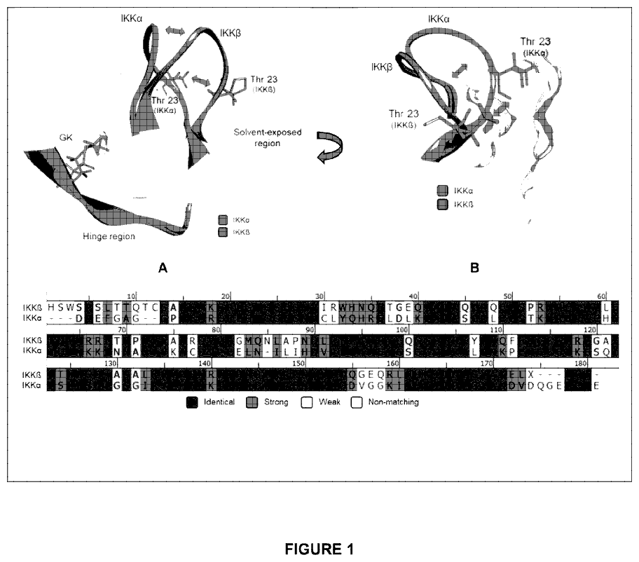

To design ligands with selectivity for IKKα over IKKβ, their ATP-binding sites were superimposed and compared to identify specific differences between the two isoforms that could be exploited. When aligning the primary sequences in this region (residues 6-180, IKKα; and 1-180, IKKβ), it is striking to see the level of homology between the two isoforms: 62.8 % sequence identity and 77.2 % sequence similarity with both having Met as the GK residue, and GK+1 /GK+3 as Glu and Cys respectively (FIG. 1). However, given the number of reported structurally diverse compounds that are selective for IKKβ over IKKα that bind in or close to the ATP-binding site [1-3 ], there are clearly differences in the two ATP-binding sites to impart such selectivity. To explore these differences, we superimposed the kinase domain of IKKβ using the 4 KIK.pdb chain B crystal structure [4 ] that contains two phosphorylated Ser residues in its activation loop, with the equivalent domain from IKKα taken from the 5 EBZ.pdb coordinates (FIG. 1). To date, no group has been able to successfully crystallise IKKα and report a high-resolution structure, but Polley and co-workers [5 ] have generated structures of IKKα in dimeric (~150 kDa) and hexameric (~450 kDa) forms using a combination of X-ray crystallography and single-particle cryoelectron microscopy to 4.5 A. This was achieved using a recombinant form of the protein where both Ser amino acids in the activation loop had been mutated to Glu residues, thus representing a constitutively activated form of the kinase that could be superimposed with the IKKβ 4 KIK crystal structure. Whilst the resolution of the IKKα structure is lower than IKKp3, encouragingly it mapped very well with the homology model we had previously reported to successfully guide structure-based inhibitor design [6].

FIG. 1 shows superimposition of the IKKα (light grey) and IKKβ(dark grey) ATP-binding sites taken from 5 EBZ.pdb and 4 KIK.pdb, respectively). A, top view of IKKα superimposed onto IKKβ binding site showing dislocation of the IKKβG-loop containing Thr 23 (dark grey) from the IKKα position (light grey), indicated by the double-headed red arrow; B, 90° rotation to show the front view of both superimposed isoforms, with the twisted Thr 23 -containing G-loop of IKKβdisplaced towards the N-lobe above the ATP binding site (double-headed arrow). C, sequence alignment of ATP binding site amino acid residues of both IKKα and IKKβ (rmsd 1.393 ), with levels of similarity colour-coded as indicated.

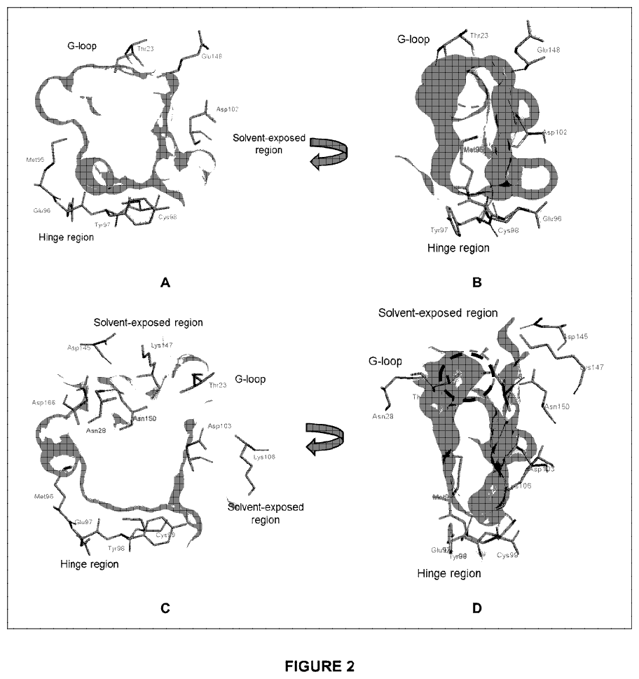

Despite the high sequence homology, a key difference between the two isoforms involves the Thr 23 -containing G-loop that is opposite the hinge-binding region, which by adopting different positions, imposes a dissimilar topography on the sites (FIG. 1). In IKKα, this G-loop forms an intact wall to enclose the site on three sides (the third involving the Met 95 GK residue shown on the left in FIG. 1A), and which offers additional binding sites to a ligand (FIG. 2A, B). In IKK3, this sequence is twisted and displaced to the upper section of the binding pocket (FIG. 2C), which removes the wall from the opposite side of the hinge-binding region seen in IKKα and exposes the site to solvent on two sides. Whilst hydrophilic groups could potentially be accommodated in this solvent region to promote binding with IKK3, the occlusion wall in IKKα offers putative interaction sites that could be exploited to favour binding with this isoform. Furthermore, the twisting of the G-loop in IKKβ to open up the site to solvent results in Thr 23 encroaching into the site itself to create an obstructive bulge that any ligand moiety accessing the solvent area would need to negotiate (FIG. 2D). Finally, because IKK3 is exposed to solvent on two sides, there is a greater number of residues proximal to the site interior available for ligand binding (Asn 28, Asp 103, Asp 145, Lys 147, Asn 150 and Asp 166, FIG. 2C) compared to that of IKKα , which only has one accessible residue; Asp 102, FIG. 2B). In summary, IKKα has a more enclosed site, only open on one side to solvent adjacent to the hinge, whereas IKKβ is more open to solvent on two sides, although with a Thr 23 protrusion that interrupts contiguous solvent access from both (FIG. 2D).

FIG. 2 shows 3 D crystal structures of the ATP binding sites of both IKKα and IKK3 (PDB ID: 5 EBZ and 4 KIK, respectively). A, top view of the IKKα binding site showing the hinge region at the base with the GK, GK+1 and GK+3 highlighted (Met 95, Glu 96 and Cys 98, respectively) and the G-loop segment at the top to form a wall opposite the hinge, with Thr 23 and Glu 148 highlighted; B, rotation by 900 to reveal the front view of the IKKα binding site from the solvent-accessible area, with the hinge region at the base. Asp 102 is the principal residue at the lip of this region available for ligand binding; C, top view of the IKKβbinding site showing the hinge region at the base and in the same position as IKKα, with Met 96, Glu 97 and Cys 99 highlighted. Thr 23 and Asn 28 from the G-loop are labelled, along with residues that line the lips of the two solvent exposed regions. D, rotation by 900 to reveal the front view of the IKKβbinding site, again with the hinge region at the bottom. The black dashed circle highlights the obstructive bulge from Thr 23 that must be negotiated to access the revealed solvent region, and the large number of residues that are presented at the lips of both solvent exposed areas (Asn 28, Asp 103, Asp 145, Lys 147, Asn 150 and Asp 166 ). The black circle in B and D shows that the equivalent position of Thr 23 in IKKα does not protrude into the site, but instead forms the back wall to block off solvent access from the top completely.

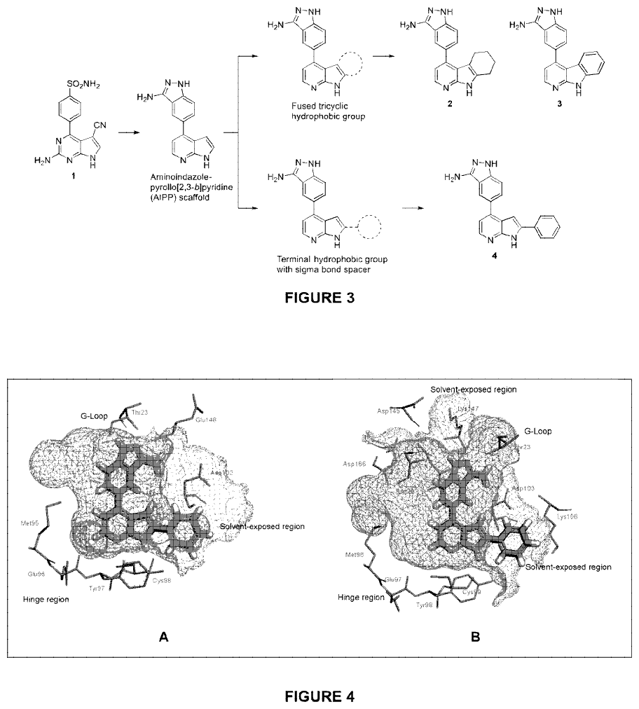

In 2008, the aminoindazole-pyrrolo[2,3 -b]pyridine (AIPP) core scaffold was identified as an inhibitor of both IKKα and IKKβ, but without revealing its potency, other than to state sub-micromolar activity against both isoforms in a cell-free, time-resolved FRET assay[7 ]. In 2017, we had identified that compound 1 SU909, a pyrrolo[2,3 -d]pyrimidine, was a selective inhibitor of IKKα that recapitulated this discrimination in U2OS cells[6 ]. To examine the pyrrolo[2,3 -b]pyridines as an alternative scaffold, we adopted the AIPP core because it had similar dimensions to 1 SU909, with HBD/HBA motifs positioned at the scaffold extremities to interact with both the hinge region and the G-loop wall of IKKα, a rationale we had previously proposed as the basis for imparting selectivity. A preliminary set of AIPP derivatives was designed to explore its utility as an IKK-targeting scaffold, whilst incorporating additional functionality through which to introduce selectivity for the IKKα isoform (2-4 SU1087, SU1266 and SU1253 ; FIG. 3). This was achieved by initially adopting two different approaches: either appending a hydrophobic moiety to the pyrrolo[2,3 -b]pyridine motif as a fused form to afford the tricyclic derivatives 2 and 3 SU1266 and SU1087, or as a phenyl substituent to introduce flexibility with the central pyrrolo[2,3 -b]pyridine moiety in 4 SU1253 . When assessed against IKKα and IKK3 using our in-house DELFIA kinase assay[6 ](Table 1 ), all three compounds showed excellent inhibitory activity against IKKα (K; 2-3 nM), which was significantly more potent than our previous hit 1 SU909 (80 nM). However, unlike 1 SU909, they had poor selectivity, inhibiting IKKβ at low nanomolar concentrations (Kᵢ 5-77 nM). This comparable activity against both isoforms could be explained by our docking studies, with all three compounds adopting similar poses in the ATP binding site of both IKKs. The pyrrolo[2,3 -b]pyridine was bound to the hinge region in the classical HBA/HBD motif with the GK+3 NH and C═O backbone, whilst the aminoindazole projected towards the top of the pocket to interact with the G-loop (FIG. 4A). Notably, in IKKα, the aminoindazole ring formed two HBD/HBA interactions with Thr 23 and Glu 148 in the occluded wall of the site, whereas in IKK3, because the G-loop is rotated and there is no equivalent wall, these interactions were absent. Moreover, in IKKβ there is a noticeable shift of the whole scaffold towards the solvent-exposed regions (FIG. 4B) for the aminoindazole to form a H-bond with Asp 103, which presumably compensates for the absence of any interaction with the G-loop wall and increases activity to a level comparable with IKKα. In both isoforms, the 2 -phenyl moiety was oriented out from the hinge towards the exposed solvent area.

FIG. 3 shows the rationale for the design of the preliminary test set of AIPPs used in this study. Tricyclic derivatives of the AIPP-based scaffold with fused hydrophobic groups were designed to study the effect of co-planarity on binding (5 a,b SU1266 and SU1087 ). A derivative with a sigma bond spacer separating the hydrophobic group from the AIPP was designed to assess flexibility (5 c SU1253 ).

TABLE 1
The Kᵢ values (nM) for the preliminary test set (5a-c) against IKKα
and IKKβ.

| | Kᵢ values (nM) | |
|---|---|---|
| Compound | IKKα | IKKβ |
| 2 | 2 | 77 |
| SU1266 | | |
| 3 | 3 | 5 |
| SU1087 | | |
| 4 | 3 | 15 |
| SU1253 | | |

FIG. 4. The predicted binding poses for 4 SU1253 with IKKα (A) and IKKβ (B). A, the pyrrolo[2,3 -b]pyridine binding motif was directed to the hinge region and exhibited two H-bond interactions with Cys 98 (2.02 and 2.14 Å). The aminoindazole binding motif was anchored to the G-loop by two H-bond interactions with Thr 23 (2.38 Å) and Glu 148 (2.21 Å) that make up the occluded wall. The 2 -phenyl ring was exposed to the solvent area and exhibited hydrophobic interactions with the non-polar amino acid residues at this site (Leu 21 and Val 151 ); B, 4 SU1253 in the IKKβactive site showed the same binding pose to that of IKKα. The pyrrolo[2,3 -b]pyridine ring had two interactions with Cys 99 (2.58 and 2.72 Å) in the hinge region, but whilst the aminoindazole was orientated towards the G-loop, it only interacted with Asp 103 (2.48 Å) in the solvent exposed area, which was occluded in IKKα.

To shift the selectivity profile towards IKKα, we selected 4 SU1253 for structural optimization based on the notion that sigma-bond rotation between the phenyl ring and the pyrrolo[2,3 -b]pyridine would inbuild the flexibility required to engage with the three dimensional array of residues nearby to exploit differences between the two isoforms. Furthermore, this would enable the inclusion of moieties that could also project into solvent and address solubility considerations. Small groups (NH₂, OH, OMe, OEt and F) were initially introduced into the phenyl ring to evaluate their impact on the selectivity profile against both IKKs (5 a-g SU1354 -SU1373, Table 2 ). Our goal was to achieve a 1:50 selectivity ratio of IKKα to IKKβ, with a minimum IKKβ inhibitory Kᵢ of 500 nM.

TABLE 2
Kᵢ values for series 1 compounds (5a-aa) against IKKα and IKKβ.

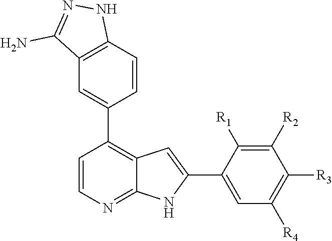

| | | | | | Kᵢ | |
|---|---|---|---|---|---|---|
| | | | | | values (nM) | |
| Compd | R₁ | R₂ | R₃ | R₄ | IKKα | IKKβ |
| 5a | OH | H | H | H | 4 | 42 |
| SU1354 | | | | | | |
| 5b | OEt | H | H | H | 8 | 86 |
| SU1368 | | | | | | |
| 5c | H | NH₂ | H | H | 5 | 28 |
| SU1304 | | | | | | |
| 5d | H | OH | H | H | 5 | 26 |
| SU1348 | | | | | | |
| 5e | H | OMe | H | H | 5 | 40 |
| SU1332 | | | | | | |
| 5f | H | OH | H | F | 4 | 42 |
| SU1384 | | | | | | |
| 5g | H | OMe | H | F | 3 | 32 |
| SU1373 | | | | | | |
| 5h SU1303 | H | 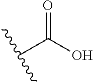 [SMILES: *C(=O)O] | H | H | 8 | 168 |
| 5i SU1294 | H | 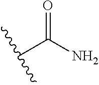 [SMILES: *C(N)=O] | H | H | 5 | 159 |
| 5j SU1288 | H | 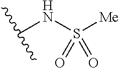 [SMILES: *NS(C)(=O)=O] | H | H | 3 | 109 |
| 5k SU1374 | H | 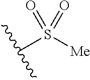 [SMILES: *S(C)(=O)=O] | H | H | 3 | 36 |
| 5l SU1317 | H | 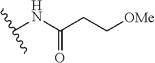 [SMILES: *NC(=O)CCOC] | H | H | 6 | 25 |
| 5m SU1316 | H | 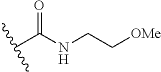 [SMILES: *C(=O)NCCOC] | H | H | 15 | 107 |
| 5n SU1550 | H | 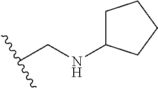 [SMILES: *CNC1CCCC1] | H | H | 10 | 191 |
| 5o SU1367 | H | 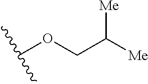 [SMILES: *OCC(C)C] | H | H | 19 | 458 |
| 5p SU1549 | H | 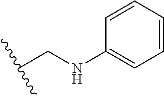 [SMILES: *CNc1ccccc1] | H | H | 35 | 470 |
| 5q | OBn | H | H | H | 57 | 1064 |
| SU1353 | | | | | | |
| 5r | H | OBn | H | H | 10 | 680 |
| SU1261 | | | | | | |
| 5s | H | H | OBn | H | 16 | 189 |
| SU1334 | | | | | | |
| 5t | H | NHTs | H | H | 10 | 1048 |
| SU1278 | | | | | | |
| 5u | H | H | NHTs | H | 4 | 67 |
| SU1283 | | | | | | |
| 5v SU1312 | H | 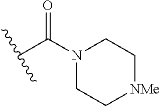 [SMILES: *C(=O)N1CCN(C)CC1] | H | H | 24 | 215 |
| 5w SU1324 | H | 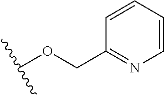 [SMILES: *OCc1ccccn1] | H | H | 16 | 402 |
| 5x SU1335 | H | 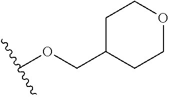 [SMILES: *OCC1CCOCC1] | H | H | 8 | 122 |
| 5y SU1336 | H | 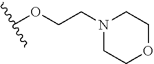 [SMILES: *OCCN1CCOCC1] | H | H | 9 | 29 |
| 5z SU1363 | H | 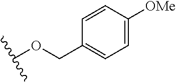 [SMILES: *OCc1ccc(OC)cc1] | H | H | 43 | 166 |
| 5aa SU1311 | H | 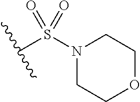 [SMILES: *S(=O)(=O)N1CCOCC1] | H | H | 25 | 149 |

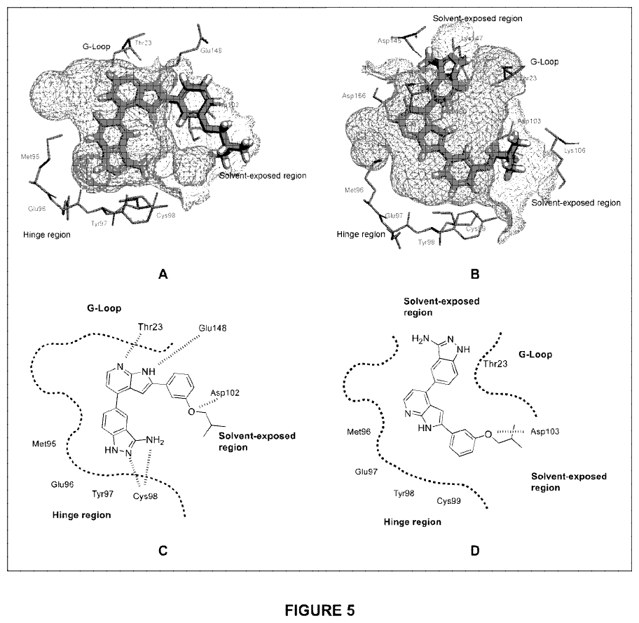

Not surprisingly, this minimal change in the structure did not improve the selectivity profile, which was supported by docking studies. 5 a-g SU1354 - SU1373 all adopted similar binding poses to 4 SU1253, with no discriminatory interactions between these substituents and the key amino acid residues lining the solvent exposed area. Crucially however, they did not compromise activity, and offered handles for further derivatisation with substituents containing appropriately positioned HBDs and HBAs that could exploit differences between each isoform (5 h-o SU1303 - SU1367 ; Table 2 ). With this set, a noticeable difference in activity between the two isoforms emerged, which appeared to be related to substituent size. Docking studies suggested that in IKKα, when the steric bulk of substituents appended to the phenyl ring was increased, the AIPP core scaffold adopted a new pose, which was essentially a 1800 flip from the 4 SU1253 pose and exemplified by 5 o SU1367 (FIG. 5A). Here, the aminoindazole is now in the hinge region forming two H-bonds with GK+1, penetrating deeper in the binding site (FIG. 5A), and the pyrrolo[2,3 -b]pyridine forms H-bonds with Thr 23 and Glu 148 in the G-loop wall. The phenyl ring and its pendant substituent point towards the solvent-exposed region and gain an additional HB interaction between the ether handle and Asp 102, potentially justifying this inversion (FIG. 5A). It appears that bulky substituents appended to the phenyl handle do not permit adoption of the 4 SU1253 pose because the opening to the solvent adjacent to the hinge in IKKα is too narrow to accommodate the bulk. Docking of this bulkier series into IKKβ can explain why activity against this isoform is reduced and selectivity improved. Significantly, no poses with the aminoindazole or the pyrrolo[2,3 -b]pyridine H-bonding with GK+1 or GK+3 were generated. We attribute this to the Thr 23 bulge in IKK3 preventing the inverted pose exemplified by 5 o SU1367 in IKKα (FIG. 5A) or the pose for 4 SU1253 (FIG. 4B) from being adopted. This protrusion will not allow the phenyl group and its bulky pendant substituent to orientate into the tunnel towards the solvent whilst concomitantly having either heterocycle in the AIPP scaffold engaging with the hinge via the conventional kinase-binding HBD/HBA motif. The only pose identified for IKKβinvolved a hook-like conformation around the displaced Thr 23 protrusion, with the aminoindazole accessing the solvent under the G-loop, the phenyl substituent accessing the solvent from the hinge, but without there being any HBD/HBA interaction with the hinge region itself (FIG. 5B), which could explain the drop in potency.

FIG. 5 shows the proposed binding orientations of 5 o SU1367 to explain its selectivity for IKKα over IKKβ(K;19 and 458 nM, respectively). A, A flipped binding orientation was observed compared to 4 SU1253 in IKKα. The aminoindazole binding motif was directed to the hinge region and showed two interactions with Cys 98 (1.89 and 2.43 Å). The pyrrolo[2,3 -b]pyridine ring displayed two interactions with Thr 23 and Glu 148 (2.05 and 2.25 Å) in the occluded wall opposite. The pendant HBA-containing substituent interacted via H-bonding Asp 102 (3.31 Å) in the solvent-exposed area; B, 5o SU1367 did not display any poses that formed conventional H-bonds to the hinge residues via either the aminoindazole or the pyrrolo[2,3 -b]pyridine moiety in IKKβ. Instead, a new orientation was generated that displayed a hook-like pose around the Thr 23 G-loop bulge that is unique to IKKβ. Here, the aminoindazole is directed into the solvent on one side of the Thr 23 protrusion and the bulky pendant substituent orientated into solvent on the other side of the bulge to form one H-bond between the ether and Asp 103 (3.34 Å). C, 2 D diagram of 5 o SU1367 in the IKKα active site showing the occluded wall in the G-loop region that affords additional interactions with the pyrrolo[2,3 -b]pyridine binding motif. H-bonds to key residues are shown as dashed lines; D, 2 D diagram of 5 o SU1367 in the IKKβ active site to illustrate the hook-like pose around the Thr 23 bulge that separates the two solvent exposed regions. The absence of any H-bonding to the hinge residues in this pose could explain the poor affinity of compounds with a bulky hydrophobic substituent for IKK3.

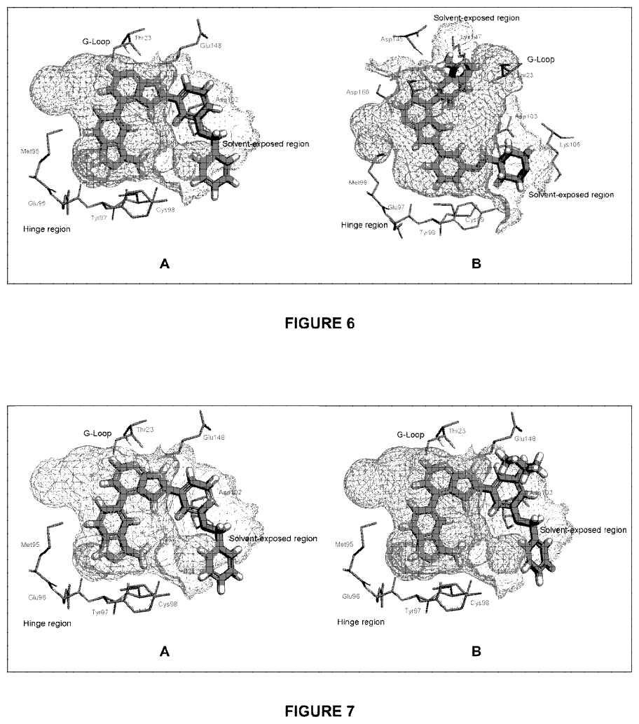

Encouraged by the observation that compounds with larger phenyl pendant substituents (5 n,o SU1550 and SU1367 ) tended to improve selectivity, we set out to explore whether increasing steric bulk could enhance the window further. We incorporated a range of moieties with varying degrees of saturation, bearing a diverse array of HBAs and HBDs (5 p-aa SU1278 - SU1283 ) to further develop the SAR and the selectivity profile. The general trend observed upon increasing the steric bulk was a more pronounced reduction in IKKβactivity, with the potency generally holding up for IKKα to markedly increase selectivity across the series. The docking results for these compounds were consistent with our earlier observations: binding to IKKα inevitably returned the flipped pose wherein the aminoindazole is bound to the hinge region (5 r SU1261, FIG. 6A) for each of the more sterically hindered analogues. Moreover, increasing the bulk of the phenyl substituents generally prevented any effective binding to the hinge region by the aminoindazole or pyrrolo[2,3 -b]pyridine in IKKβ, other than the unfavourable hook-like conformation around the displaced Thr 23 protrusion seen for 5 o SU1367, and no H-bonding to the hinge region. This pose was consistent for those compounds that displayed the lowest activity against IKKβ (5 o-r, t, w SU1367, SU1278, SU1549, SU1353, SU1324, and SU1261 ).

FIG. 6 shows the predicted 3 D binding pose for 5 r SU1261 with IKKα (A) and 5× SU1335 with IKKβ (B) A, the aminoindazole binding motif of 5 r SU1261 was situated at the hinge region of IKKα, exhibiting two key interactions with Cys 98 (1.99 and 2.56 Å), with the pyrrolo[2,3 -b]pyridine positioned at the G-Loop opposite and displaying two key interactions with Thr 23 (2.04 Å) and Glu 148 (2.39 Å). As with 5 o SU1367, the pendant phenyl ring resided in the solvent exposed region, with the benzyl ether oxygen atom hydrogen bonding to Asp 102 (2.52 Å); B, the hook-like binding pose was observed for 5× SU1335 in IKK3, with the aminoindazole moiety making interactions with Thr 23 (2.65 Å), Gly 24 (3.04 Å), and Asn 28 (2.00 Å). Of note were the additional interactions that were observed with the terminal polar pyran group forming an interaction with Lys 106 (2.99 Å), which may compensate for an absence of H-bonding with the hinge region in IKK3.

A number of analogues did exhibit good IKKβ inhibition despite increased steric hindrance, for example 51,m,x,y SU1317, SU1316, SU1335 and SU1336 . Notably, these derivatives all possess terminal polar groups in their pendant phenyl substituent, and whilst the docking studies consistently returned the hook-like pose in IKKβ described for 5 o,t,p,q,w SU1367, SU1278, SU1549, SU1353, SU1324, and SU1261 (and exemplified by 5 o SU1367 in FIG. 5B), these terminal groups were able to form additional H-bonding interactions with either the protonated Lys106 residue in the case of 5× SU1335 (FIG. 6B), or the Tyr 98 side chain in the case of 5 y SU1336 situated in the solvent exposed region. We propose that these additional interactions could compensate for those not seen with the hinge for 5 o,t,p,q,w SU1367, SU1278, SU1549, SU1353, SU1324, and SU1261 to explain the improved activity with IKKβ.

Shifting the phenyl pendant substituent to the para-position (e.g 5 s,u SU1334 and SU1283 ) tended to improve IKKβactivity to reduce selectivity and was therefore not extensively pursued. From a docking perspective, the altered geometry of the ligand caused by para substitution generated a new pose in IKKβ which could explain this increase in activity. Here, the aminoindazole was positioned along the hinge region to H-bond with GK+1, the pyrrolo[2,3 -b]pyridine accessing the solvent adjacent to the hinge, and the para-pendant substituted phenyl ring projected upwards into the solvent exposed region below the G-loop to form an additional H-bond with the Thr 23 bulge via the ether (5 s SU1334 ) or sulfonamide (5 u SU1283 ). Both compounds adopted the flipped pose for IKKα that had been returned across the series.

With a rationale for selectivity established, we next sought to improve the physicochemical properties of the series via the incorporation of solubilising groups and additional heteroatoms in the central phenyl ring (6 a-1 SU1371 - 1621, Table 3 ). 5 r SU1261 was selected as the starting point for the optimisation of the of the series, owing to its activity and selectivity being recapitulated in cells. The introduction of polar functional groups and additional nitrogen atoms was explored, with a view to reducing lipophilicity and improving aqueous solubility. Modifications such as linker length extension, heteroatom choice and placement were concurrently explored to see if the potency and specificity of the ligand could be further refined.

TABLE 3
Kᵢ values of series 2 compounds (6a-l) against IKKα and IKKβ.

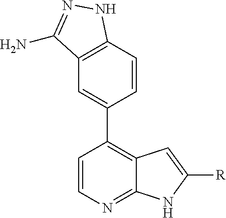

| | | Kᵢ values (nM) | |
|---|---|---|---|
| Cmpd | R | IKKα | IKKβ |
| 6a SU1371 | 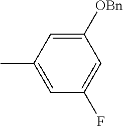 | 59 | 5029 |
| 6b SU1358 | 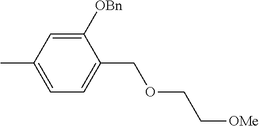 | 29 | 1215 |
| 6c SU1365 | 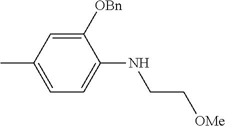 | 26 | 1532 |
| 6d SU1350 | 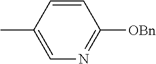 | 12 | 22 |
| 6e SU1337 | 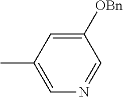 | 21 | 1550 |
| 6f SU1394 | 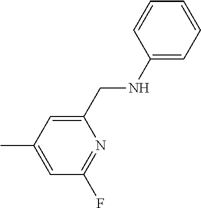 | 33 | 503 |
| 6g SU1349 | 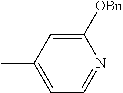 | 16 | 3352 |
| 6h SU1615 | 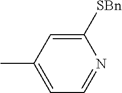 | 5 | 1180 |
| 6i SU1380 | 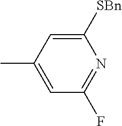 | 127 | 3000 |
| 6j SU1602 | 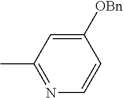 | 25 | 2300 |
| 6k SU1628 | 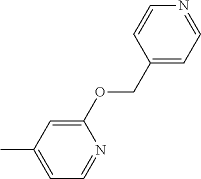 | 6 | 12 |
| 6l SU1621 | 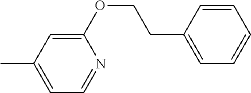 | 42 | 1600 |

The addition of heteroatoms and polar moieties generally maintained potency for IKKα and decreased IKKβ inhibition, which ultimately improved the selectivity of these analogues. Again, similarities with the prior docking results were observed, with bulky meta-substituents on the phenyl ring positioned in the same solvent-exposed orientation in IKKα, allowing significant interaction between the AIPP core with the hinge and G-loop wall in the flipped pose to facilitate potent inhibition (6 g SU1349, FIG. 7A). Furthermore, most analogues had poor activity against IKK3, which could generally be accounted for in silico by the familiar, unfavourable hook-like conformation shown in FIGS. 5B and D for 5 o SU1367 being predominantly replicated in IKKβ with no H-bonding to the hinge.

FIG. 7 shows proposed 3 D interactions of 6 g SU1349 (A) and 6 c SU1365 (B) with the IKKα active site. A, the aminoindazole motif of 6 g SU1349 was positioned at the hinge region, displaying two H-bond interactions with Cys 98 (2.00 and 2.62 Å), the pyrrolo[2,3 -b]pyridine H-bonded with Thr 23 (2.04 Å) and Glu 148 (2.46 Å), and the ether showed an HB interaction between the oxygen atom and Asp 102 (2.49 Å); B, the aminoindazole motif of 6 c SU1365 was positioned at the hinge region, displaying two interactions with Cys 98 (1.90 and 2.39 Å) and the pyrrolo[2,3 -b]pyridine interacted with the G-loop wall via Thr 23 (2.02 Å) and Glu 148 (2.26 Å). The pendant phenyl ring was positioned in the solvent exposed region, making a Tr-anion interaction with Asp 102 (2.52 Å), with the benzyl ether oxygen atom hydrogen bonding to Asp 102 (2.75 Å). The additional solubilising alkyl ether substituent was accommodated in the solvent exposed region.

Compounds with a supplementary solubilising long-chain polar group (6 b,c SU1358 and SU1365 ) demonstrated similar selectivity profiles that could be explained by our model. The solvent-exposed area in IKKα was large enough for them to consistently adopt the standard flipped pose across the series associated with inhibition (6 c SU1365, FIG. 7B). In IKK3, the increased steric bulk that arises from a disubstituted arrangement in two trajectories could not accommodate the Thr 23 bulge under any conditions and generated no viable binding poses.

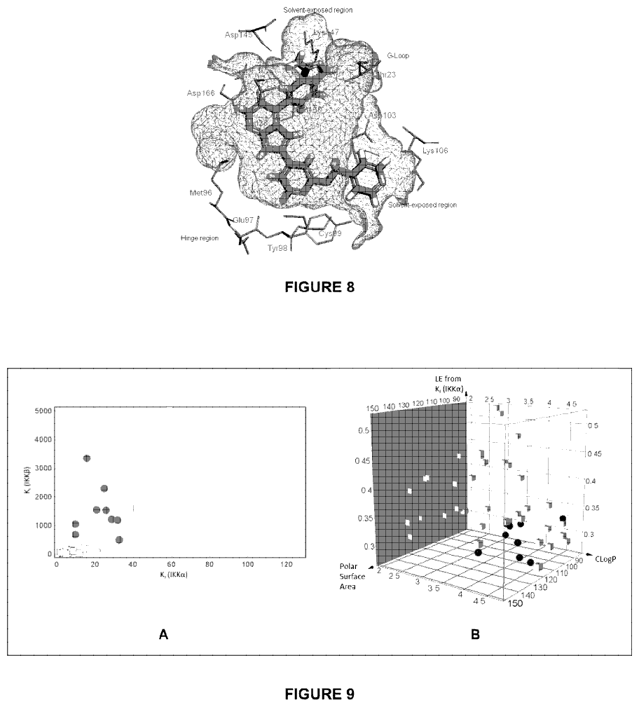

Whilst the addition of heteroatoms and polar moieties generally maintained selectivity for these analogues, there was one exception: 6 k SU1628 displayed potent inhibition against IKKβdespite possessing a bulky substituent on the phenyl ring. However, said substituent is a polar pyridyl group, which replicates the poorer selectivity seen for 51,m,x,y SU1317, SU1316, SU1335 and SU1336, all which contain H-bonding pendant groups. Furthermore, the docked pose of 6 k SU1628 was similar, with an additional H-bonding interaction seen with Lys 106, which serves to improve the affinity of the unfavourable hook-like pose (FIG. 8). Together these data suggest that bulky substituents with a terminal polar functionality that can hydrogen bond to the IKKβ isoform should be avoided if selectivity is to be maintained. Finally, the para-substituted analogue 6 d SU1350 also displayed reduced selectivity, and replicated the docking pose seen for 5 s,u SU1334 and SU1283.

FIG. 8 shows the 3 D binding pose of 6 k SU1628 with IKKβ. The familiar hook-like binding pose was observed, with the aminoindazole moiety H-bonding with Thr 23 (2.64 Å) and Asn 28 (2.12 Å), but no interaction with the hinge residues. However, an additional interaction was observed between the terminal pyridyl nitrogen and Lys 106 (2.44 Å), which could compensate for the absence of any H-bonding with the hinge.

### Chemistry

The synthetic strategy for accessing the compounds described herein began with the organoiridium(I) catalysed C—H activation of commercially available fluorobenzonitrile 7, giving rise to boronic ester intermediate 8 (Scheme 1 ) [8 ]. This was followed by a ring closure using hydrazine, which proceeds via nucleophilic aromatic substitution at the aryl fluoride and nucleophilic attack at the nitrile carbon to afford the key aminoindazole intermediate 9 (Scheme 1 ). The final step in the preparation of the initial set of AIPP derivatives 2-4 was a Suzuki-Miyaura cross-coupling between boronic ester intermediate 9 and the commercially sourced pyrrolo[2,3 -b]pyridine aryl chlorides 10-12 (Scheme 1 ) [9 ].

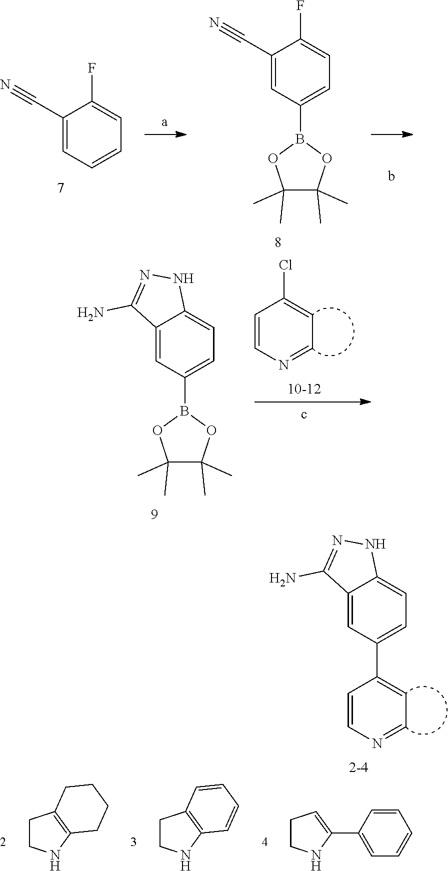

Preparation of compounds 5 was achieved via the same route as the initial compound set 2-4 (Scheme 2 ). Aryl chlorides 15 were prepared via an additional Suzuki reaction of 4 -chloro-2 -iodo-pyrrolo[2,3 -b]pyridine (13 ) with a range of boronic acids 14 (Scheme 2 ), with selectivity for the pyrrolo[2,3 -b]pyridine 2 -position achieved through the reduced reactivity of aryl chlorides compared with aryl iodides within the same scaffold [10 ]. This enabled two Suzuki coupling reactions to be performed selectively in sequence by judicious choice of catalyst systems with varying activity i.e., triphenylphosphine palladium catalysis at the iodide moiety, followed by chloride-directing palladium catalysis using a ferrocene-based ligand. A number of these pyrrolo[2,3 -b]pyridines were further functionalised before the final coupling; Specifically, anilines 15 c and 15 ac were subjected to alkylation conditions to afford amide and sulfonamide intermediates 151, 15 t, and 15 u, carboxylic acid 15 h was functionalised via amide bond formation to afford intermediate 15 m, and the benzaldehyde functional handle of 15 ab underwent reductive amination, giving rise to amines 15 n and 15 p. As before, the final coupling was carried out between the synthesised aryl halides 15 and boronic ester 9 to afford the final compounds 5 displayed in Table 3, possessing varying degrees of steric bulk (Scheme 2 ).

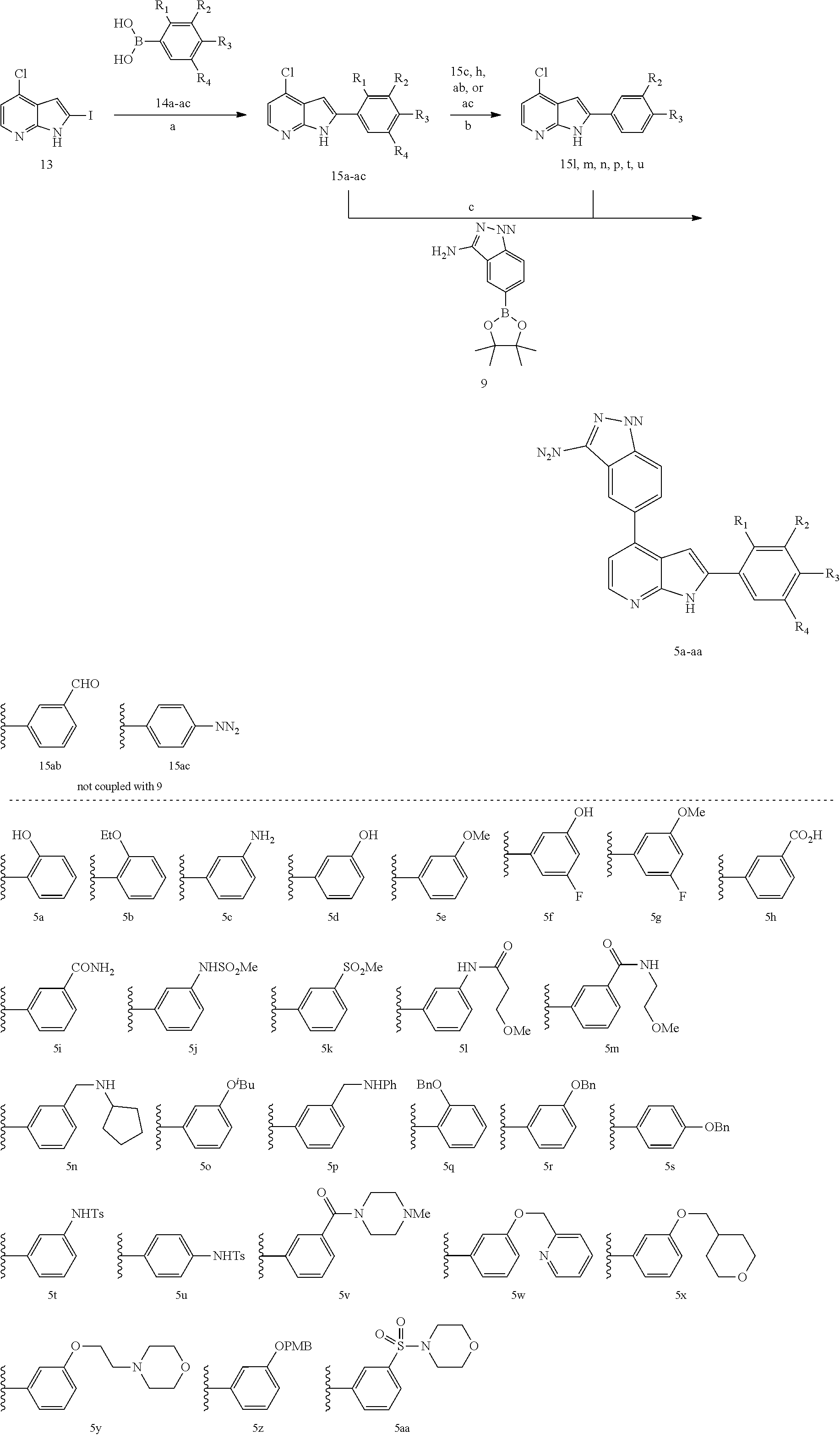

Considering analogues 6 a-i, designed with increased solubility in mind, synthesis followed a similar route to those described above. A selection of phenyl and pyridyl boronic acids 16 a-i were coupled to the aryl iodide moiety of 13, giving rise to intermediates 17 . Following this, the scaffold was decorated at the chloride moiety via a Suzuki coupling with key intermediate 9 to afford compounds 6 (Scheme 3 ).

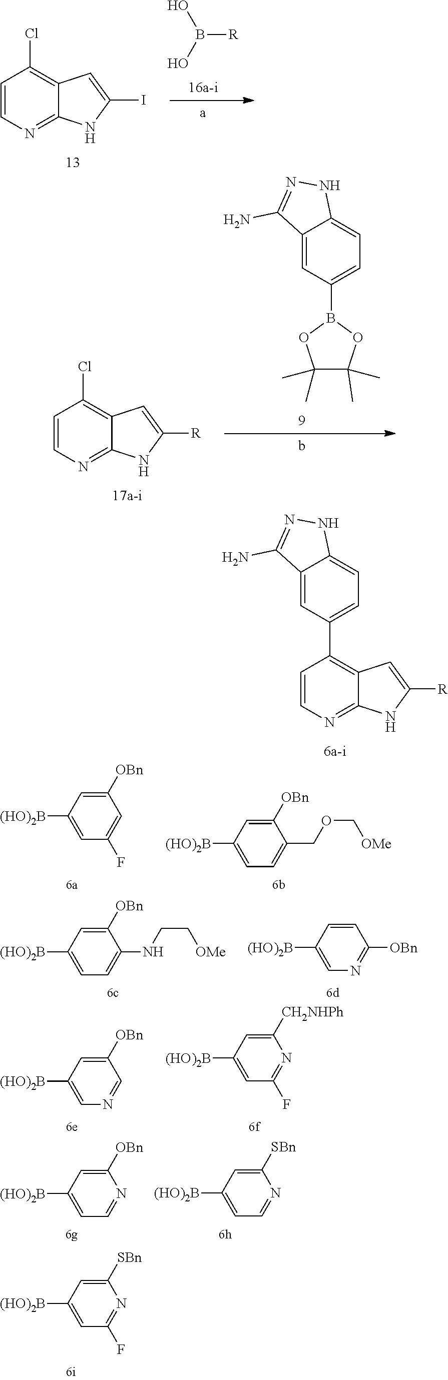

Intermediates in the route towards compounds 6 j-1 required bespoke synthesis, which began from commercially available disubstituted pyridines 18 (Scheme 4 ). Intermediates 20 were prepared via S_{N}2 halide displacement of benzyl bromide in the case of 20 a, or nucleophilic aromatic substitution of the aromatic fluoride in the case of 20 b and 20 c, then subsequently subjected to Suzuki-Miyaura borylation conditions to afford intermediates 21 (Scheme 4 ) [11 ]. A protecting group strategy was utilised to aid in purification to afford intermediates 23, employing either methoxymethyl (MOM) chloride or t-butyloxycarbonyl (Boc) anhydride, which were then iodinated via lithium-halogen exchange using n-BuLi, giving rise to protected intermediates 24 . These were then subjected to consecutive Suzuki couplings at the two- and four-position halide moieties sequentially with boronate esters 21 to first generate intermediates 25, and then boronate ester 9 to yield the final target compounds 6 j-1 (Scheme 4 ).

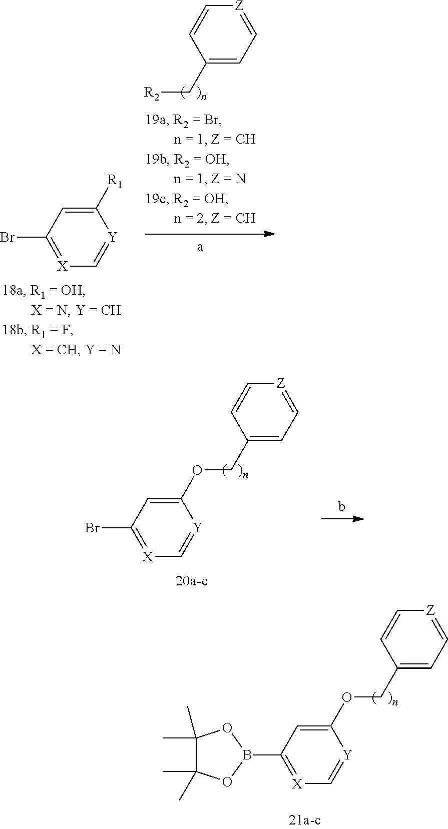

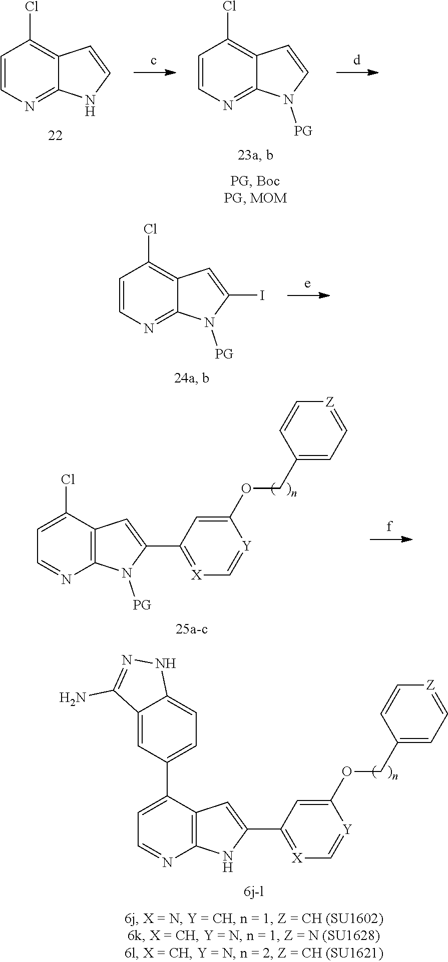

### Physicochemical/DMPK Analysis

We next examined our series using Datawarrior, an open-source software package for the generation and analysis of physicochemical attributes. Firstly, compounds were plotted with respect to inhibition of IKKα versus IKKβ that included an initial dataset filter, dependent on an IKKα Kᵢ <40 nM and IKKβ Kᵢ >500 nM (FIG. 9A).

FIG. 9 shows graphical correlation between the activity and the calculated physicochemical properties. A, IKKα versus IKKβselectivity analysis. Compounds with superior potency and selectivity profile highlighted in dark grey; B, Physicochemical property analysis. Compounds with superior potency and selectivity profile highlighted in blue.

With an initial filter applied for potency and selectivity, the data could be arranged according to the key physicochemical properties: CLog P, topological polar surface area (tPSA), and Ligand efficiency (LE), with compounds that possessed adequate potency and selectivity highlighted in dark grey (FIG. 9B).

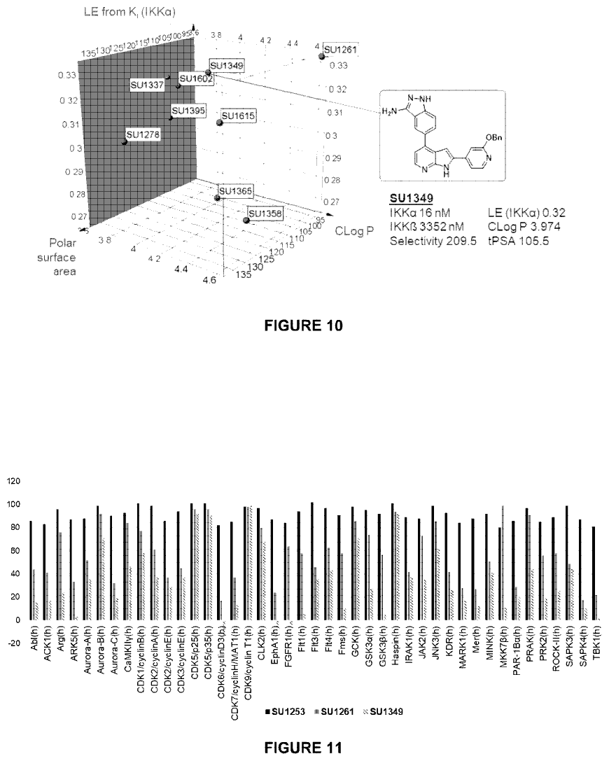

Removing the compounds with an undesirable selectivity profile, the filtered datapoints fall into a narrow region of chemical space, possessing a CLog P of ca. 3.5-4.5, a tPSA of 100-120 Å² (with two outliers at 90 and 140 Å² ), and LE values approximately at the desirable benchmark of 0.3 for further development. All the compounds that met our selectivity and potency criteria possessed bulky hydrophobic meta-substituents, with seven of the nine derivatives bearing either a benzyl ether or thioether and a pyridyl group, the most notable example being 6 g SU1349 which boasts a 209 -fold selectivity for IKKα over IKKβ(FIG. 10).

FIG. 10 shows physicochemical property analysis.

Modification of 5 r SU1261 to those derivatives in FIG. 10, whilst generally improving selectivity, only improved solubility marginally (Table 4 ) despite the introduction of solubilising ether groups used in the development of erlotinib (6 b,c SU1358 and SU1365 ), and herein lies the problem to further progress this series: to date, incorporating polar functionality in the solvent-exposed pendant group has improved solubility, but significantly compromised the in vitro biochemical selectivity for IKKα (vide supra). This is clearly demonstrated by comparing 6 g SU1349 with 6 k SU1628 - the latter has the requisite solubility but is equipotent against both isoforms, whereas the former has the essential selectivity profile, but poorer solubility. However, whilst 6 g SU1349 had lower solubility than 6 k SU1628, it was the most soluble in the series that displayed selectivity, and furthermore had a sufficiently low in vitro murine clearance to be progressed to an in vivo PK evaluation (Table 5 ).

TABLE 4
Turbidimetric solubility and murine hepatocyte clearance of selected
compounds from the series.

| | | Mouse hepatocyte | Mouse |
|---|---|---|---|
| | Solubility | clearance (μL/min/106 | hepatocyte half- |
| Compd | (μM) | cells) | life (min) |
| 5r | <1 | 38 | 36 |
| SU1261 | | | |
| 6b | 3.75 | ND | ND |
| SU1358 | | | |
| 6c | 2 | ND | ND |
| SU1365 | | | |
| 6e | 6.5 | ND | ND |
| SU1337 | | | |
| 6f | 3.75 | ND | ND |
| SU1394 | | | |
| 6g | 20 | 27 | 51 |
| SU1349 | | | |
| 6h | 2 | ND | ND |
| SU1615 | | | |
| 6j | 2 | ND | ND |
| SU1602 | | | |
| 6k | >100 | 11 | 121 |
| SU1628 | | | |

TABLE 5
in vivo murine PK parameters for 6 g SU1349.

| | | Mean PK parameters | | | | | |
|---|---|---|---|---|---|---|---|
| Adminis- | Dose | | Terminal | Vₛₛ | Cₘₐₓ | | |
| tration | (mg/ | Clearance | t_{1/2} | (L/ | (ng/ | Tₘₐₓ | F |
| route | kg) | (mL/min/kg) | (hr) | kg) | mL) | (hr) | (%) |
| I.V. | 10 | 16.7 | 4.18 | 0.39 | | | |
| I.P. | 10 | | 3.02 | | 199 | 0.44 | 9.3 |

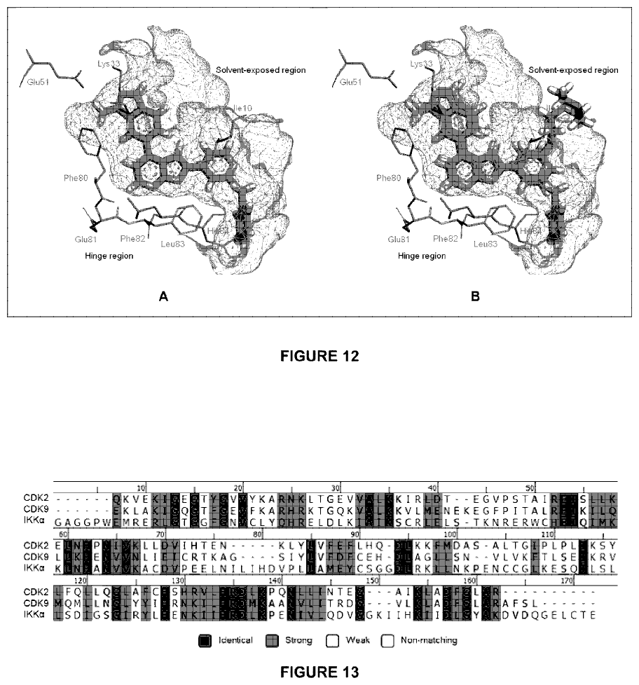

We profiled three compounds from the series across the kinome (FIG. 11). Our original pan-IKK inhibitor 4 SU1253 proved to be very promiscuous, inhibiting 44 kinases from a panel of 231 by >80 % at 1 μM. The introduction of the pendant benzyloxy substituent to generate 5 r SU1261 markedly reduced off-target kinase inhibition to 10 kinases (>80 % at 1 μM), most notably CDK5, CDK9, haspin, and the stress-activated kinases MKK7 P and PRAK. Interestingly, exchanging the 2 -phenyl pyrrolo[2,3 -b]pyridine substituent of 5 r SU1261 for the 2 -pyridin-4 -yl group in 6 g SU1349 not only improved solubility and clearance, but also significantly reduced off-target inhibition of MKK73 and PRAK, although not CDK5 and CDK9 . To identify the structural drivers for CDK inhibition, we obtained X-ray crystal structures of 5 y and 6 g SU1336 and SU1349 with CDK-2 (FIG. 12A) to potentially identify possible routes towards introducing selectivity for IKKα over the CDK enzymes as well as IKKβ. The CDK2 and CDK9 active sites are comparable with IKKβ in the sense that they have an accessible pocket that is readily open to solvent on two sides and moreover, do not possess an equivalent Thr 23 bulge, resulting in a larger volume to accommodate bulky substituents, whilst allowing H-bonding to the hinge residues (FIG. 13). All three derivatives form complexes with CDK2 with the pyrrolo[2,3 -b]pyridine H-bonding to GK+3 (Leu 83 ) and the aminoindazole projected towards the G-loop (FIG. 12A), forming H-bonds with Glu 51 and Lys 33 at the back of the pocket. The adoption of the 4 SU1253 -type orientation, rather than the flipped pose observed in IKKα, is probably facilitated by Tr-stacking between the aminoindazole and the GK residue, which in the CDKs is Phe, rather than Met. Notably, the positioning of the pendant side chain is altered dramatically, depending on the nature of the substituent and the potential interactions made. For 6 g SU1349, the hydrophobic pendant benzyloxy group points out into solvent, but can form face-to-edge Tr-Tr interactions with Phe 82 and His 84 from the hinge. Exchanging the hydrophobic pendant benzyloxy group for the morpholinoethyl-containing substituent in 5 y SU1336 removes any direct interaction with these hinge residues, preferring to sit in a more solvent-exposed position, as befits a basic side chain that will be protonated at physiological pH (FIG. 12B).

FIG. 11 shows kinases showing significant % inhibition by 4 SU1253, 5 r SU1261 and 6 g SU1349 at 1 μM from a panel of 253 kinases. Black, 4 SU1253 ; grey, 5 r SU1261 ; diagonal stripes, 6 g SU1349. 4 SU1253 showed >80 % inhibition for all 46 kinases shown. 5 r SU1261 inhibited fewer kinases compared to that of 6 g SU1253 (exhibited >80 % inhibition for 10 kinases). 6 g SU1349 exhibited a significant reduction in off-target inhibition (>80 % inhibition for only 4 kinases). All tested compounds (4 SU1253, 5 r SU1261 and 6 g SU1349 ) inhibited CDK5 and CDK9 with >80 %.

FIG. 12 shows: A, Crystal structure of 15 g SU1349 with CDK2 . The aminoindazole moiety was directed to the G-loop, forming HBs with Glu51 and Lys33 (3.09 and 2.92 A, respectively), the pyrollo[2,3 -b]pyridine resided at the hinge region H-bonding to Leu83 (2.94 Å), and the pendant benzyl ether was positioned into the solvent-exposed region flat along the protein wall, forming π-π interactions with Phe82 and His84 ; B, Overlapped crystal structures of 13 k SU1336 and 15 g SU1349 with CDK2 . Whilst the AIPP motif adopts a similar position in the ATP-interior for both compounds, the morpholineoethyl side chain of 13 k SU1336 adopts a completely different position to the benyloxy group of 15 g SU1349 . In the former, this basic side chain is fully exposed to the solvent rather than interacting with specific residues in the kinase.

FIG. 13 shows sequence alignment of the ATP binding site amino acid residues of IKKα (residues 10-180 ), CDK2 (residues 5-150 ) and CDK9 (residues 20-180 ) (alignment rmsd 1.081 and 1.634 ). The homology analysis showed 16.3 % sequence identity and 40.1 % sequence similarity between these 3 kinases (IKKα, CDK2 and CDK9 ), with levels of similarity colour-coded as indicated.

One key observation from these CDK complexes is the presence of a lipophilic pocket side chain between the hinge and the adjacent solvent-exposed region, made up of the side chains of lie 10, Phe 82 and Leu 134 . In CDK9, an equivalent hydrophobic pocket is formed from Ile 25, Phe 105 and Leu 156 . These residues are positioned perfectly to make Tr-alkyl and Tr-sigma interactions with the central 2 -phenyl/pyridyl ring present in our series. These additional interactions are possible across our compound series, and when combined with the H-bonding interactions anchoring the central AIPP core to the hinge region, provide a potential explanation for the observed off-target activity against CDK isoforms.

### EXPERIMENTAL SECTION

General. Unless otherwise stated, all commercially available reagents and solvents used were obtained from Sigma-Aldrich, Fluorochem Fisher Scientific, Acros, Alfa Aesar, Apollo scientific and Advanced ChemBlocks and used without further purification. Air- or moisture-sensitive reactions were carried out under argon or nitrogen atmosphere. Microwave reactions were carried out using a Biotage Initiator system. Thin-layer chromatography (TLC) was carried out on aluminium-backed SiO₂ μlates (Merck, silica gel 60, F₂₅4 ) and spots visualised using ultra-violet light (254 nm) or by staining with potassium permanganate. All tested compounds were determined to be ≥95 % purity by LC-MS and analytical HPLC unless otherwise stated. Flash chromatography was performed using a Biotage SP4 automated chromatography system using silica stationary phase (Fisher Scientific, 60 Å, 35-70 micron; detection wavelength: 254 nm; monitoring: 280 nm) and the mobile phase used are detailed in the text. Reverse phase HPLC purifications were conducted on Shimadzu Prominance HPLC using a semi-preparative (50×21.2 mm) Luna 5 μm C18 column at 40° C.; flow rate: 6 ml/min; detection wavelength: 254 nm eluting with an acetonitrile/water gradient with 0.1 % TFA. NMR spectra were recorded on either a Bruker Avance3 /DPX400 (400 MHz), Bruker DRX500 (500 MHz), Bruker AV400 (400 MHz), Bruker AV500 HD (500 MHz) or Bruker AV600 (600 MHz) instrument and analysed using Advanced Chemistry Development Labs (ACD/labs) NMR processor 12.00 or MestReNova 10.0 software. Chemical shifts (δ) are recorded in parts per million (ppm) relative to an internal solvent reference (tetramethylsilane) and coupling constants (J) in Hertz (Hz). Splitting patterns were indicated as singlet (s), broad singlet (br s), doublet (d), doublet of doublet (dd), triplet (t), quartet (q) and multiplet (m). LCMS was carried out on an Agilent Technologies 1220 series LC system with Agilent 6100 series quadrupole mass spectrometer in ESI/APCI mode. Separation was achieved with an Agilent Eclipse C18 4.6×50 mm column; flow rate:1 ml/min; detection:254 nm; sample volume:10 μl; mobile phase: acetonitrile/5 mM ammonium acetate: water/5 mM ammonium acetate; 5 %, 1.48 min; 5-100 %, 8 min; 100 %, 13.5 min; 100-5 %, 16.5 min; 18 min. HRMS was carried out on an Exactive (Thermo scientific) or LTQ orbitrap (Thermo scientific).

Synthesis of 2 -fluoro-5 -(4,4,5,5 -tetramethyl-1,3,2 -dioxaborolan-2 -yl)benzonitrile (8 ). A solution of bis(pinacolato)diboron (1.3 g, 5.2 mmol), 4,4 ′-di-tert-butyl-2,2 ′-dipyridyl (43 mg, 0.16 mmol) and (1,5 -cyclooctadiene)(methoxy)iridium(I) dimer (106 mg, 0.16 mmol) in anhydrous MTBE (10 mL) in a sealed vial (20 mL) was stirred at rt for 1 h. A solution of 2 -fluorobenzonitrile (1, 0.6 g, 5 mmol) in anhydrous MTBE (1 mL) was added. The reaction mixture was allowed to stir at 80° C. for 18 h. The reaction mixture was cooled, filtered through celite and evaporated under reduced pressure. The crude residue was used in the next step without further purification.

Synthesis of 5 -(4,4,5,5 -tetramethyl-1,3,2 -dioxaborolan-2 -yl)-1 H-indazol-3 -amine (9 ). To a solution of compound 8 (1.98 g, 8 mmol) in EtOH (100 mL), hydrazine hydrate (2.4 mL, 2.47 g, 39 mmol, 50-60 %) was added and the reaction was refluxed for 30 h. The solvent was evaporated under reduced pressure. The residue was triturated with a mixture of EtOAc and petroleum ether (1:1, 12 mL), filtered and washed with water and petroleum ether 60-80 % to give the titled product 3 as a yellow solid (1.4 g, 67 %). ¹H NMR (400 MHz, DMSO-d₆ ) δ ppm 12.70 (s, 1 H), 8.17 (s, 1 H), 7.49 (d, J=8.35 Hz, 1 H), 7.18 (d, J=7.91 Hz, 1 H), 5.46 (s, 2 H), 1.30 (s, 12 H). ¹³ C NMR (100 MHz, DMSO-d₆ ) δ ppm 150.34, 143.38, 132.01, 129.35, 114.66, 109.23, 83.74, 25.31 . LC-MS: exact mass calculated for C₁₃H₁₈BN₃O₂ : 259.12, found 260.1 (M+1 )⁺.

General procedure for synthesis of 5 -(substituted pyridin-4 -yl)-1 H-indazol-3 -amine (2 -4 ). To a suspension of 4 -chloro-pyridine derivatives (10-12, 0.35 mmol), compound 9 (0.136 g, 0.525 mmol) and [1,1 ′-bis(di-tert-butylphosphino)ferrocene]dichloro palladium(II) catalyst (0.011 g, 0.0175 mmol) in EtOH (1 mL) and water (1 mL), a solution of K₃ PO₄ was added (1 M, 0.88 mL) and the reaction mixture was heated to 120° C. for 20 h. The reaction mixture was cooled, diluted with EtOAc and washed with water and brine. The organic layer was dried over anhydrous sodium sulfate and concentrated under reduced pressure. The crude residue was purified by column chromatography (10 % MeOH in EtOAc) to afford the titled products (2-4 ).

5 -(6,7,8,9 -Tetrahydro-5 H-pyrido[2, 3 -b]indol-4 -yl)-1 H-indazol-3 -amine (2 ). Beige solid (73 mg, 69 %). ¹H NMR (400 MHz, DMSO-d₆ ) δ 11.46 (s, 1 H), 11.27 (s, 1 H), 8.07 (d, J=4.9 Hz, 1 H), 7.78 (s, 1 H), 7.33 (d, J=8.6 Hz, 1 H), 7.29 (d, J=8.6 Hz, 1 H), 6.86 (d, J=4.9 Hz, 1 H), 5.43 (s, 2 H), 2.71 (t, J=5.8 Hz, 2 H), 2.20 (t, J=5.2 Hz, 2 H), 1.81-1.77 (m, 2 H), 1.66-1.50 (m, 2 H). HRMS (ESI): exact mass calculated for C₁₈H₁₇N₅ : 303.1552, found 304.1557 (M+1 )⁺.

5 -(9 H-pyrido[2,3 -b]indol-4 -yl)-1 H-indazol-3 -amine (3 ). White powder (36 mg, 35 %). ¹ H NMR (400 MHz, DMSO-d₆ ): δ 11.90 (br s, 1 H), 11.62 (br s, 1 H), 8.44 (d, J=5.0 Hz, 1 H), 8.05 (s, 1 H), 7.57 (d, J=8.1 Hz, 1 H), 7.54 (dd, J=8.7, 1.4 Hz, 1 H), 7.51 (d, J=8.1 Hz, 1 H), 7.44 (d, J=8.5 Hz, 1 H), 7.42-7.38 (m, 1 H), 7.11 (d, J=5.0 Hz, 1 H), 7.02-6.98 (m, 1 H), 5.47 (br s, 2 H). LC-MS: exact mass calculated for C₁₈H₁₃N₅ : 299.12, found 300.3 (M+1 )⁺.

5 -(2 -Phenyl-1 H-pyrrolo[2,3 -b]pyridin-4 -yl)-1 H-indazol-3 -amine (4 ). Off-white solid (50 mg, 44 %). ¹H NMR (500 MHz, DMSO-d₆ ) δ 12.27 (s, 1 H), 12.05 (s, 1 H), 8.27-8.24 (m, 2 H), 7.99 (d, J=8.5 Hz, 1 H), 7.74 (s, 1 H), 7.55-7.49 (m, 2 H), 7.40-7.34 (m, 3 H), 7.22 (d, J=8.5 Hz, 1 H), 7.17 (d, J=1.9 Hz, 1 H), 5.73 (s, 2 H).

General procedure for synthesis of 4 -chloro-2 -(substituted phenyl)-1 H-pyrrolo[2,3 -b]pyridine (15 a-ac). A suspension of 4 -chloro-2 -iodo-7 -azaindole (0.343 g, 1.23 mmol), substituted phenyl boronic acid (14 a-ac, 1.52 mmol), K₂ CO₃ (0.483 g, 3.49 mmol) and bis(triphenylphosphine) palladium(II) chloride (0.074 g, 0.105 mmol) in dioxane (3 mL) and water (2 mL) was degassed under nitrogen. The reaction mixture was allowed to stir at 100° C. for 20 h. The reaction mixture was cooled to rt and extracted between EtOAc (5 mL) and water (3 mL). The organic layer wash washed with brine (2×3 mL), dried over anhydrous sodium sulfate and concentrated under reduced pressure. The crude residue was used in the next step without further purification unless otherwise stated below.

4 -Chloro-2 -(2 -ethoxyphenyl)-1 H-pyrrolo[2,3 -b]pyridine (15 b). The resulting solid was purified by recrystallization using DCM and hexane to afford the titled compound as an orange solid (207 mg, 62 %). ¹H NMR (DMSO-d₆ ): δ 12.13 (br s, 1 H), 8.17 (d, J=5.2 Hz, 1 H), 7.89 (dd, J=1.6, 0.6 Hz, 1 H), 7.39-7.36 (m, 1 H), 7.19 (d, J=5.2 Hz, 1 H), 7.16 (s, 1 H), 7.08-7.06 (m, 2 H), 4.21 (q, J=6.9 Hz, 2 H), 1.46 (t, J=7.0 Hz, 3 H).

3 -(4 -Chloro-1 H-pyrrolo[2,3 -b]pyridin-2 -yl)-5 -fluorophenol (15 f). The resulting solid was purified by column chromatography (90 % EtOAc in petroleum ether) to afford the titled compound as a yellow solid (69 mg, 21.5 %). ¹H NMR (DMSO-d6): 6 12.50 (br s, 1 H), 10.13 (s, 1 H), 8.19 (d, J=4.8 Hz, 1 H), 7.30 (dt, J=1.8, 10.0 Hz, 1 H), 7.23-7.21 (m, 2 H), 6.98 (d, J=2.4 Hz, 1 H), 6.59 (dt, J=2.2, 10.4 Hz, 1 H). LC-MS: exact mass calculated for C₁₃H₈³⁵ ClFN₂0: 262.03, found 263.13 (M+1 )⁺.

N-(3 -(4 -Chloro-1 H-pyrrolo[2,3 -b]pyridin-2 -yl)phenyl)methanesulfonamide (15 j). The crude solid was purified by column chromatography (90 % EtOAc in petroleum ether) to afford the titled compound as a pale yellow solid (0.216 g, 64.7 %). ¹H NMR (DMSO-d₆ ): δ 12.62 (br s, 1 H), 9.88 (br s, 1 H), 8.19 (d, J=5.2 Hz, 1 H), 7.75-7.71 (m, 2 H), 7.45 (t, J=16.0 Hz, 1 H), 7.22-7.20 (m, 2 H), 6.88 (d, J=2.0 Hz, 1 H), 3.10 (s, 3 H). HRMS (ESI): exact mass calculated for C₁₄H₁₃O₂N₃₃₅ CIS: 322.0412, found 322.0410 (M+1 )⁺.

4 -Chloro-2 -(3 -(methylsulfonyl)phenyl)-1 H-pyrrolo[2,3 -b]pyridine (15 k). The resulting solid was purified by column chromatography (90 % EtOAc in petroleum ether) to afford the titled compound as an off-white solid (245 mg, 62 %). ¹H NMR (DMSO-d₆ ): δ 12.73 (br s, 1 H), 8.56 (t, J=1.6 Hz, 1 H), 8.35 (dt, J=1.2, 8.2 Hz, 1 H), 8.24 (d, J=5.3 Hz, 1 H), 7.92 (dt, J=1.2, 8.2 Hz, 1 H), 7.77 (t, J=7.8 Hz, 1 H), 7.26 (dd, J=5.0 Hz, 1 H), 7.24 (s, 1 H), 3.33 (s, 3 H). LC-MS: exact mass calculated for C₁₄H₁₂₃5 ClN₃O₂ S: 321.03, found 322.3 (M+1 )⁺.

4 -Chloro-2 -(3 -isobutoxyphenyl)-1 H-pyrrolo[2,3 -b]pyridine (15 o). The resulting solid was purified by recrystallization using DCM and hexane to afford the titled compound as an orange solid (166 mg, 45 %). ¹H NMR (DMSO-d₆ ): δ 12.48 (br s, 1 H), 8.17 (d, J=5.2 Hz, 1 H), 7.58 (s, 1 H), 7.56 (s, 1 H), 7.37 (t, J=8.0 Hz, 1 H), 7.20 (d, J=5.2 Hz, 1 H), 7.04 (d, J=2.4 Hz, 1 H), 6.94-6.92 (m, 1 H), 3.85 (d, J=6.4 Hz, 2 H), 2.08-2.05 (m, 1 H), 1.02 (d, J=6.4 Hz, 6 H).

2 -(2 -(Benzyloxyphenyl)-4 -chloro-1 H-pyrrolo[2,3 -b]pyridine (15 q). The resulting solid was triturated with hexane and Et₂O to afford the titled compound as a light solid (378 mg, 92 %). ¹H NMR (DMSO-d₆ ): δ 12.19 (br s, 1 H), 8.18 (d, J=7.5 Hz, 1 H), 7.92 (dd, J=2.0, 8.8 Hz, 1 H), 7.54 (d, J=7.5 Hz, 1 H), 7.42 (t, J=7.0 Hz, 1 H), 7.36-7.34 (m, 2 H), 7.28 (d, J=8.5 Hz, 1 H), 7.17 (d, J=5.0 Hz, 1 H), 7.09 (t, J=7.5 Hz, 1 H), 7.06 (s, 1 H), 5.32 (s, 2 H).

3 -(4 -Chloro-1 H-pyrrolo[2,3 -b]pyridin-2 -yl)phenyl)(4 -methylpiperazin-1 -yl)methanone (15 v). The resulting solid was triturated with hexane and filtered through a pad of celite, concentrated under reduced pressure and dried to afford the titled compound as light solid (405 mg, 93 %). ¹H NMR (DMSO-d₆ ): δ 12.57 (br s, 1 H), 8.19 (d, J=4.0 Hz, 1 H), 8.08 (d, J=6.8 Hz, 1 H), 8.00 (s, 1 H), 7.55 (t, J=6.2 Hz, 1 H), 7.37 (d, J=6.0 Hz, 1 H), 7.22 (d, J=4.0 Hz, 1 H), 7.11 (s, 1 H), 3.66 (br s, 2 H), 3.36 (br s, 2 H), 2.39 (br s, 2 H), 2.23 (br s, 2 H), 2.21 (s, 3 H).

4 -Chloro-2 -(3 -(pyridin-2 -ylmethoxy)phenyl)-1 H-pyrrolo(2,3 -b]pyridine (15 w). The crude residue was purified by column chromatography (90 % EtOAc in petroleum ether) to give the titled compounds as brown solid (380 mg, 92 %). ¹H NMR (400 MHz, DMSO-d₆ ) δ 12.52 (s, 1 H), 8.60 (d, J=4.6 Hz, 1 H), 8.18 (d, J=5.2 Hz, 1 H), 7.88-7.84 (m, 1 H), 7.73 (s, 1 H), 7.61 (d, J=7.7 Hz, 1 H), 7.58 (d, J=7.8 Hz, 1 H), 7.45-7.37 (m, 1 H), 7.36 (d, J=7.2 Hz, 1 H), 7.21 (d, J=5.2 Hz, 1 H), 7.06-7.03 (m, 2 H), 5.30 (s, 2 H). LC-MS: exact mass calculated for C₁₉H₁₄ ³⁵ClN₃O: 335.08, found 336.1 (M+1 )⁺.

2 -(3 -((Tetrahydro-2 H-pyran-4 -yl)methoxy)phenyl)-4 -chloro-1 H-pyrrolo[2,3 -b]pyridine (15×). The crude residue was triturated with 50 % EtOAc in petroleum ether to afford the titled product as brown solid (336 mg, 80 %). “H NMR (400 MHz, DMSO-d₆ ) δ 12.51 (s, 1 H), 8.17 (d, J=5.2 Hz, 1 H), 7.58-7.56 (m, 2 H), 7.39-7.35 (m, 1 H), 7.20 (d, J=5.2 Hz, 1 H), 7.05 (d, J=2.0 Hz, 1 H), 6.94 (d, J=7.2 Hz, 1 H), 3.93 (d, J=6.8 Hz, 2 H), 3.89 (dd, J=11.2, 2.8 Hz, 2 H), 3.37 (dd, J=12.4, 1.6 Hz, 2 H), 2.08-2.00 (m, 1 H), 1.72 (d, J=11.6 Hz, 2 H), 1.42-1.31 (m, 2 H). LC-MS: exact mass calculated for C₁₈H₁₉³⁵ClN₂02: 342.11, found 343.3 (M+1 )⁺.

4 -(2 -(3 -(4 -Chloro-1 H-pyrrolo[2,3 -b]pyridin-2 -yl)phenoxy)ethyl)morpholine (15 y). The crude residue was purified by flash chromatography (10 % MeOH in EtOAc) to obtain the titled compound as beige solid (136 mg, 31 %). ¹H NMR (400 MHz, DMSO-d6) 6 12.49 (s, 1 H), 8.17 (d, J=5.2 Hz, 1 H), 7.60 (s, 1 H), 7.58 (d, J=8.0 Hz, 1 H), 7.39-7.35 (m, 1 H), 7.20 (d, J=5.2 Hz, 1 H), 7.06 (d, J=2.0 Hz, 1 H), 6.95 (dd, J=8.1, 2.0 Hz, 1 H), 4.19 (t, J=5.8 Hz, 2 H), 3.61 -3.58 (m, 4 H), 2.73 (t, J=5.8 Hz, 2 H), 2.51-2.48 (m, 4 H). LC-MS: exact mass calculated for C₁₉H₂₀³⁵ClN₃O₂ : 357.12, found 358.1 (M+H)⁺.

4 -Chloro-2 -(3 -((4 -methoxybenzyl)oxy)phenyl)-1 H-pyrrolo[2,3 -b]pyridine (15 z). The resulting solid was purified by column chromatography (90 % EtOAc in petroleum ether) and triturated with Et₂O to afford the titled compound as an orange solid (228 mg, 51 %). ¹ H NMR (DMSO-d₆ ): δ 12.48 (br s, 1 H), 8.17 (d, J=5.2 Hz, 1 H), 7.68 (t, J=2.0 Hz, 1 H), 7.58 (d, J=8.0 Hz, 1 H), 7.43-7.41 (m, 3 H), 7.21 (d, J=5.2 Hz, 1 H), 7.05 (s, 1 H), 6.98-6.96 (m, 3 H), 5.13 (s, 2 H), 3.75 (s, 3 H).

4 -((3 -(4 -Chloro-1 H-pyrrolo[2,3 -b]pyridin-2 -yl)phenyl)sulfonyl)morpholine (15 aa). The resulting solid was triturated with boiling MeOH, filtered and dried to afford the titled compound as brown solid (88 mg, 19 %). ¹H NMR (DMSO-d₆ ): δ 12.83 (br s, 1 H), 8.37-8.35 (m, 1 H), 8.23 (d, J=4.8 Hz, 1 H), 7.78-7.75 (m, 4 H), 7.25 (d, J=5.2 Hz, 1 H), 7.23 (d, J=2.0 Hz, 1 H), 3.66-3.65 (m, 4 H), 2.96-2.94 (m, 4 H).

2 -(4 -(Benzyloxy)phenyl)-4 -chloro-1 H-pyrrolo[2,3 -b]pyridine (15 s). The crude residue was purified by column chromatography (90 % EtOAc in petroleum ether) followed by trituration to the resulting solid by a mixture of hexane/Et₂O to afford the titled compound as an off-white solid (177 mg, 43 %). ¹H NMR (DMSO-d₆ ): δ 12.38 (br s, 1 H), 8.13 (d, J=5.5 Hz, 1 H), 7.94 (d, J=8.5 Hz, 1 H), 7.47 (d, J=7.0 Hz, 2 H), 7.41 (t, J=7.5 Hz, 2 H), 7.35 (d, J=7.5 Hz, 1 H), 7.18 (d, J=5.0 Hz, 1 H), 7.12 (d, J=8.5 Hz, 1 H), 6.87 (d, J=2.0 Hz, 1 H), 5.19 (s, 2 H).

Synthesis of N-(3 -(4 -chloro-1 H-pyrrolo[2,3 -b]pyridin-2 -yl)phenyl)-3 -methoxypropanamide (151 ). A solution of compound 15 c (0.10 g, 0.4 mmol) and 3 -methoxypropanoic acid (37 μL, 0.4 mmol) in DMF (5 mL) was allowed to stir at rt for 5 min. HCTU (495 mg, 1.2 mmol) and trimethylamine (168 μL, 1.2 mmol) were added and the solution allowed to stir at rt for 18 h. The reaction mixture was cooled, extracted between EtOAc and water. The organic layer was dried over anhydrous sodium sulfate and removed under reduced pressure. The resulting residue was purified by column chromatography (1 % Et₃ N and 10 % MeOH in EtOAc) to give the titled product as a pale-yellow solid (95 mg, 72 %). ¹H NMR (500 MHz, DMSO-d₆ ) δ 12.54 (s, 1 H), 10.04 (s, 1 H), 7.89-7.86 (m, 2 H), 7.66 (d, J=7.8 Hz, 1 H), 7.57 (d, J=7.8 Hz, 1 H), 7.47 (t, J=10.0 Hz, 1 H), 7.22 (d, J=6.5 Hz, 1 H), 6.81 (d, J=1.8 Hz, 1 H), 3.65 (t, J=8.0 Hz, 2 H), 3.27 (s, 3 H), 2.04 (t, J=8.0 Hz, 2 H). LC-MS: exact mass calculated for C₁₇H₁₆³⁵ClN₃O₂ : 329.09, found 330.3 (M+H)⁺.

Synthesis of N-(3 -(4 -chloro-1 H-pyrrolo[2,3 -b]pyridin-2 -yl)phenyl)-4 -methylbenzenesulfonamide (15 t). Tosyl chloride (136 μL, 1 mmol) was added to a solution of compound 15 c (0.244 g, 1.04 mmol) and triethyl amine (0.3 mL, 2 mmol) in anhydrous DCM (5 mL) at 0° C. The reaction mixture was allowed to stir at rt for 18 h. The reaction mixture was washed with saturated solution of sodium hydrogen carbonate (5 mL) and brine (5 mL), dried over anhydrous sodium sulfate and removed under reduced pressure. The crude residue was purified by column chromatography (90 % EtOAc in petroleum ether) to afford the titled compound as a pale-yellow solid (301 mg, 73 %). ¹H NMR (DMSO-d₆ ): δ 12.57 (br s, 1 H), 10.36 (br s, 1 H), 8.19 (d, J=5.2 Hz, 1 H), 7.71 (d, J=8.4 Hz, 2 H), 7.65-7.64 (m, 2 H), 7.38-7.36 (m, 3 H), 7.22 (d, J=5.2 Hz, 1 H), 7.08-7.05 (m, 1 H), 6.74 (d, J=2.0 Hz, 1 H), 2.33 (s, 3 H).

Synthesis of 3 -(4 -chloro-1 H-pyrrolo[2,3 -b]pyridin-2 -yl)-N-(2 -methoxyethyl)benzamide (15 m). A solution of compound 15 h (0.11 g, 0.4 mmol) and 2 -methoxyethylamine (35 μL, 0.4 mmol) in DMF (5 mL) was allowed to stir at rt for 5 min. HCTU (495 mg, 1.2 mmol) and trimethylamine (168 μL, 1.2 mmol) were added. The reaction mixture was allowed to stir at rt for 18 h. The solvent was then removed under high vacuum and the resulting residue was used in the next step without further purification.

General procedure for synthesis of N-(3 -(4 -chloro-1 H-pyrrolo[2,3 -b]pyridin-2 -y1 )benzyl)-N-substituted amine (15 n,p). A solution of compound 15 ab (0.12 g, 0.46 mmol), appropriate amine (0.6 mmol), sodium triacetoxyborohydride (0.14 g, 0.69 mmol) and acetic acid (0.036 mL, 0.59 mmol) in dimethylacetamide (2 mL) at rt for 48 h. The reaction mixture was poured into 1 M sodium carbonate solution, stirred in an ice bath for 3 h and filtered.

N-[3 -(4 -Chloro-7 -azaindole)benzyl]-N-cyclopentylamine (15 n). The collected solid was purified by column chromatography (10 % MeOH in EtOAc) to afford the desired compound as a white solid (102 mg, 68 %), ¹H NMR (400 MHz, DMSO-d₆ ) δ 12.49 (br s, 1 H), 8.17 (d, J=5.27 Hz, 1 H), 7.97 (s, 1 H), 7.84 (d, J=7.47 Hz, 1 H), 7.41 (t, J=7.69 Hz, 1 H), 7.35 (d, J=7.91 Hz, 1 H), 7.21 (d, J=5.27 Hz, 1 H), 6.99 (s, 1 H), 3.74 (s, 2 H), 3.03-3.01 (m, 1 H), 1.73-1.71 (m, 2 H), 1.64-1.62 (m, 2 H), 1.47-1.44 (m, 2 H), 1.37-1.35 (m, 2 H). LC-MS: exact mass calculated for C₁₉H₂₀ ³⁵ClN₃: 325.13, found 326.2 (M+1 )⁺.

N-[3 -(4 -Chloro-7 -azaindole)benzyl]-N-phenylamine (15 p). The collected solid was purified by column chromatography (60 % EtOAc in petroleum ether) to afford the desired compound as a white solid (118 mg, 77 %). ⁴ H NMR (400 MHz, DMSO-d₆ ) δ 12.54 (br s, 1 H), 8.17 (d, J=5.27 Hz, 1 H), 8.04 (s, 1 H), 7.85 (d, J=7.47 Hz, 1 H), 7.43 (t, J=7.69 Hz, 1 H), 7.38 (d, J=7.91 Hz, 1 H), 7.21 (d, J=5.27 Hz, 1 H), 7.08-7.02 (m, 2 H), 6.97 (s, 1 H), 6.62 (d, J=7.47 Hz, 2 H), 6.51 (t, J=7.25 Hz, 1 H), 6.27 (t, J=5.93 Hz, 1 H), 4.32 (d, J=6.15 Hz, 2 H). ¹³ C NMR (100 MHz, DMSO-d₆ ) δ 150.07, 149.17, 144.36, 143.96, 141.78, 140.04, 134.03, 131.42, 129.53, 129.41, 128.02, 125.38, 124.48, 120.44, 116.43, 112.94, 95.56, 47.05 . LC-MS: exact mass calculated for C₂₀H₁₆³⁵ClN₃: 333.10, found 334.1 (M+1 )⁺.

Synthesis of N-(4 -(4 -chloro-1 H-pyrrolo[2,3 -b]pyridin-2 -yl)phenyl)-4 -methylbenzenesulfonamide (15 u). Tosyl chloride (136 μL, 1 mmol) was added to a solution of compound 15 ac (0.244 g, 1 mmol) and triethyl amine (0.3 mL, 2 mmol) in anhydrous DCM (5 mL) at 0° C. The reaction mixture was allowed to stir at rt for 18 h. The reaction mixture was quenched with saturated solution of sodium hydrogen carbonate (5 mL). The organic layer was washed with brine (5 mL), dried over anhydrous sodium sulfate and removed under reduced pressure. The crude residue was purified by column chromatography (90 % EtOAc in petroleum ether) to afford the titled compound as an off-white solid (199 mg, 50 %). ¹H NMR (DMSO-d6): 6 12.39 (br s, 1 H), 10.47 (br s, 1 H), 8.13 (d, J=5.2 Hz, 1 H), 7.85 (dd, J=2.0, 6.8 Hz, 2 H), 7.69 (dd, J=1.6, 6.4 Hz, 2 H), 7.36 (d, J=8.0 Hz, 2 H), 7.17 (dd, J=0.8, 6.4 Hz, 3 H), 6.87 (d, J=2.4 Hz, 1 H), 2.33 (s, 3 H).

General procedure for synthesis of 5 -(2 -(substituted phenyl)-1 H-pyrrolo[2.3 -b]pyridin-4 -yl)-1 H-indazol-3 -amine (5 a-aa). K₃ PO₄ (1 M, 1.2 mL) was added to a suspension of compounds 15 (0.37 mmol), compound 9 (0.146 g, 0.56 mmol) and [1,1 ′-bis(di-tert-butylphosphino)ferrocene]dichloro palladium(II) catalyst (0.028 g, 0.04 mmol) in EtOH (3 mL).

The reaction mixture was heated to 120° C. for 20 h. The reaction mixture was cooled to rt, diluted with EtOAc, washed with water and brine. The organic layer was dried over anhydrous sodium sulfate and concentrated under reduced pressure.

2 -(4 -(3 -Amino-1 H-indazol-5 -yl)-1 H-pyrrolo[2,3 -b]pyridin-2 -yl)phenol (5 a). The crude residue was purified by column chromatography (80 % EtOAc in petroleum ether) to give the titled product as off-white solid (10 mg, 8 %). ¹H NMR (DMSO-d₆ ): δ 11.67 (br s, 1 H), 11.54 (br s, 1 H), 10.17 (br s, 1 H), 8.23 (d, J=5.2 Hz, 1 H), 8.19 (s, 1 H), 7.85 (dd, J=1.6, 7.6 Hz, 1 H), 7.69 (dd, J=1.6, 8.4 Hz, 1 H), 7.40 (d, J=8.8 Hz, 1 H), 7.28 (s, 1 H), 7.17-7.15 (m, 2 H), 6.98 -6.96 (m, 1 H), 6.92-6.89 (m, 1 H), 5.50 (br s, 2 H). HRMS (ESI): exact mass calculated for C₂₀H₁₅ON₅: 341.1349, found 342.1346 (M+1 )⁺.

5 -(2 -(2 -Ethoxyphenyl)-1 H-pyrrolo[2,3 -b]pyridin-4 -yl)-1 H-indazol-3 -amine (5 b). The crude residue was purified by column chromatography (90 % EtOAc in petroleum ether) to afford the titled compound as an off-white solid (56 mg, 41 %). ¹H NMR (DMSO-de): 6 11.81 (br s, 1 H), 11.54 (br s, 1 H), 8.25 (d, J=5.0 Hz, 1 H), 8.22 (s, 1 H), 7.92 (dd, J=2.0, 8.0 Hz, 1 H), 7.72 (dd, J=1.5, 9.0 Hz, 1 H), 7.39-7.37 (m, 2 H), 7.34-7.32 (m, 1 H), 7.18 (d, J=5.0 Hz, 1 H), 7.14 (d, J=6.4 Hz, 1 H), 7.05 (t, J=5.8 Hz, 1 H), 5.50 (br s, 2 H), 4.18 (q, J=5.5 Hz, 2 H), 1.44 (t, J=7.0 Hz, 3 H). HRMS (ESI): exact mass calculated for C₂₂H₁₉N₅O: 369.1662, found 370.1660 (M+1 )⁺.

5 -(2 -(3 -Aminophenyl)-1 H-pyrrolo[2,3 -b]pyridin-4 -yl)-1 H-indazol-3 -amine (5 c). The crude residue was purified by flash chromatography (90 % EtOAc in petroleum ether) to obtain the titled product as beige solid (21 mg, 17 %). ¹H NMR (500 MHz, DMSO-d₆ ) δ 12.45 (s, 1 H), 8.37 (s, 1 H), 8.30 (d, J=7.8 Hz, 1 H), 7.84 (d, J=8.0 Hz 2 H), 7.54-7.47 (m, 3 H), 7.34 (t, J=8.0 Hz, 1 H), 7.26 (d, J=5.0 Hz, 1 H), 7.15 (s, 1 H), 5.57 (s, 2 H). LC-MS: exact mass calculated for C₂₀H₁₆ N: 340.14, found 341.20 (M+1 )⁺.

3 -(4 -(3 -Amino-1 H-indazol-5 -yl)-1 H-pyrrolo[2,3 -b]pyridin-2 -yl)phenol (5 d). The crude residue was purified by column chromatography (10 % MeOH in EtOAc) to obtain the titled product as brown solid (49 mg, 39 %). ¹H NMR (400 MHz, DMSO-d₆ ) δ 12.16 (s, 1 H), 11.56 (s, 1 H), 9.56 (s, 1 H), 8.25-8.24 (m, 2 H), 7.71 (dd, J=8.5, 2.1 Hz, 1 H), 7.34 (s, 1 H), 7.44-7.38 (m, 2 H), 7.28-7.24 (m, 1 H), 7.19 (d, J=5.3 Hz, 1 H), 6.77 (d, J=8.0 Hz, 1 H), 7.09 (d, J=2.2 Hz, 1 H), 5.57 (s, 2 H). HRMS (ESI): exact mass calculated for C₁₉H₁₅N₅O: 341.1301, found 341.1401 (M+1 )⁺.

5 -(2 -(3 -Methoxyphenyl)-1 H-pyrrolo[2,3 -b]pyridin-4 -yl)-1 H-indazol-3 -amine (5 e). The crude residue was purified by column chromatography (10 % MeOH in EtOAc) to afford the titled compound as a brown solid (58 mg, 44 %). ¹H NMR (500 MHz, DMSO) δ 12.43 (s, 1 H), 8.39 (s, 1 H), 8.33 (d, J=5.1 Hz, 1 H), 7.89 (d, J=8.7 Hz, 1 H), 7.61-7.57 (m, 2 H), 7.53 (d, J=8.7 Hz, 1 H), 7.41 (t, J=8.2 Hz, 1 H), 7.28 (d, J=5.2 Hz, 1 H), 7.26 (s, 1 H), 6.96 (dd, J=8.2, 2.4 Hz, 1 H), 3.87 (s, 3 H). LC-MS: exact mass calculated for C₂,H₁₇N₅O: 355.14, found 356.14 (M+1 )⁺.

3 -(4 -(3 -Amino-1 H-indazol-5 -yl)-1 H-pyrrolo[2,3 -b]pyridin-2 -yl)-5 -fluorophenol (50 . The crude residue was purified by column chromatography (10 % MeOH in EtOAc) to afford the titled compound as yellow solid (29 mg, 21.9 %) ¹H NMR (DMSO-d₆ ): δ 12.35 (br s, 1 H), 10.11 (s, 1 H), 8.35 (s, 1 H), 8.32 (d, J=4.8 Hz, 1 H), 7.85 (dd, J=1.6, 8.8 Hz, 1 H), 7.49 (d, J=8.4 Hz, 1 H), 7.29-7.26 (m, 1 H), 7.26 (d, J=4.8 Hz, 1 H), 7.24 (t, J=1.6 Hz, 1 H), 7.20 (d, J=2.0 Hz, 1 H). HRMS (ESI): exact mass calculated for C₂₀H₁₄FN₅O: 359.1255, found 360.1252 (M+1 )⁺.

5 -(2 -(3 -Fluoro-5 -methoxyphenyl)-1 H-pyrrolo[2,3 -b]pyridin-4 -yl)-1 H-indazol-3 -amine (5 g). The crude solid was purified by column chromatography (90 % EtOAc in petroleum ether) to give the titled compound as a yellow solid (62 mg, 45 %). ¹H NMR (400 MHz, DMSO-d₆ ) δ 12.31 (s, 1 H), 11.59 (s, 1 H), 8.29 (d, J=4.9 Hz, 1 H), 8.24 (s, 1 H), 7.73 (d, J=8.7 Hz, 1 H), 7.50-7.43 (m, 2 H), 7.40 (d, J=8.7 Hz, 1 H), 7.32 (s, 1 H), 7.22 (d, J=5.0 Hz, 1 H), 6.85-6.77 (m, 1 H), 5.58 (s, 2 H), 3.86 (s, 3 H). LC-MS: exact mass calculated for C₂1H₁₆FN₅O: 373.13, Found 374.3 (M+1 )⁺.

3 -(4 -(3 -Amino-1 H-indazol-5 -yl)-1 H-pyrrolo[2,3 -b]pyridin-2 -yl)benzoic acid (5 h). The crude residue was purified by column chromatography (90 % EtOAc in petroleum ether) to obtain the titled compound as beige solid (34 mg, 25 %). ¹H NMR (500 MHz, DMSO-d₆ ) δ 12.48 (s, 1 H), 8.53 (s, 1 H), 8.34 (s, 1 H), 8.30 (d, J=5.0 Hz, 1 H), 8.23 (d, J=8.1 Hz, 1 H), 7.92 (d, J=7.0 Hz, 1 H), 7.85 (d, J=8.5 Hz 1 H), 7.61 (t, J=7.8 Hz, 1 H), 7.49 (d, J=8.7 Hz, 1 H), 7.26 -7.24 (m, 2 H), 5.54 (s, 2 H). LC-MS: exact mass calculated for C₂₁H₁₅N₅O₂: 369.12, found 370.2 (M+1 )⁺.

3 -(4 -(3 -Amino-1 H-indazol-5 -yl)-1 H-pyrrolo[2,3 -b]pyridin-2 -yl)benzamide (5 i). The crude solid was purified by column chromatography (10 % MeOH in EtOAc) to obtain the titled compound as beige solid (23 mg, 17 %). ¹H NMR (500 MHz, DMSO-d₆ ) δ 12.48 (s, 1 H), 8.53 (s, 1 H), 8.34 (s, 1 H), 8.30 (d, J=5.0 Hz, 1 H), 8.23 (d, J=8.1 Hz, 1 H), 7.92 (d, J=7.0 Hz, 1 H), 7.85 (d, J=8.5 Hz 1 H), 7.61 (t, J=7.8 Hz, 1 H), 7.49 (d, J=8.7 Hz, 1 H), 7.26-7.23 (m, 2 H), 5.54 (s, 2 H). LC-MS: exact mass calculated for C₂₁H₁₆N₆O: 368.12, found 369.2 (M+1 )⁺.

N-(3 -(4 -(3 -Amino-1 H-indazol-5 -yl)-1 H-pyrrolo(2,3 -b]pyridin-2 -y1 )phenyl)methanesulfonamide (5 j). The crude residue was purified by column chromatography (10 % MeOH in EtOAc) and triturated with Et₂O to afford the titled compound as an off-white solid (14 mg, 9 %). ¹H NMR (DMSO-de): 6 12.32 (br s, 1 H), 11.57 (br s, 1 H), 9.83 (br s, 1 H), 8.27 (d, J=5.2 Hz, 1 H), 7.70-7.68 (m, 3 H), 8.23 (s, 1 H), 7.42 (q, J=8.3 Hz, 2 H), 7.19-7.16 (m, 2 H), 7.07 (d, J=2.0 Hz, 1 H), 5.56 (br s, 2 H), 3.09 (s, 3 H). HRMS (ESI): exact mass calculated for C₂₁H₁₈N₆O₂ S: 418.1285, found 419.1287 (M+1 )⁺.

5 -(2 -(3 -(Methylsulfonyl)phenyl)-1 H-pyrrolo[2,3 -b]pyridin-4 -yl)-1 H-indazol-3 -amine (5 k). The crude solid was purified by column chromatography (10 % MeOH/EtOAc) to afford the titled compound as an off-white solid (34 mg, 23 %). ¹H NMR (DMSO-d₆ ): δ 12.43 (br s, 1 H), 11.57 (br s, 1 H), 8.27 (t, J=1.2 Hz, 1 H), 8.32-8.30 (m, 2 H), 8.25 (s, 1 H), 7.87 (d, J=6.4 Hz, 1 H), 7.75-7.73 (m, 2 H), 7.42 (d, J=6.8 Hz, 1 H), 7.37 (d, J=1.6 Hz, 1 H), 7.23 (d, J=4.0 Hz, 1 H), 3.31 (s, 3 H). HRMS (ESI): exact mass calculated for C₂₁ Hj₇N₅O₂ S: 403.1132, found 404.1172 (M+1 )⁺.

N-(3 -(4 -(3 -Amino-1 H-indazol-5 -yl)-1 H-pyrrolo(2,3 -b]pyridin-2 -yl)phenyl)-3 -methoxypropanamide (51 ). The crude residue was purified by column chromatography (90 % EtOAc in petroleum ether) to obtain the titled compound as beige solid (27 mg, 17 %). ¹ H NMR (500 MHz, DMSO-d₆ ) δ 12.45 (s, 1 H), 10.08 (s, 1 H), 8.32 (d, J=6.50 Hz, 1 H), 8.15 (s, 1 H), 7.88-7.85 (m, 1 H), 7.67 (d, J=7.8 Hz, 1 H), 7.60 (d, J=7.8 Hz, 1 H), 7.54 (d, J=8.0 Hz, 1 H), 7.43 (t, J=10.0 Hz, 1 H), 7.25 (d, J=6.5 Hz, 1 H), 7.06 (d, J=1.8 Hz, 1 H), 3.63 (t, J=8.0 Hz, 2 H), 3.25 (s, 3 H), 2.58 (t, J=8.0 Hz, 2 H). LC-MS: exact mass calculated for C₂₄H₂₂N₆O₂: 426.18, found 427.20 (M+1 )⁺.

3 -(4 -(3 -Amino-1 H-indazol-5 -yl)-1 H-pyrrolo[2,3 -b]pyridin-2 -yl)-N-(2 -methoxyethyl)benzamide (5 m). The resulting residue was purified by column chromatography (1 % Et₃ N and 10 % MeOH in EtOAc) to give the titled product as an off white solid (139 mg, 88 %). ¹H NMR (500 MHz, DMSO-d₆ ) δ 12.35 (s, 1 H), 8.59 (s, 1 H), 8.44 (d, J=5.0 Hz, 1 H), 8.31 (d, J=8.1 Hz, 1 H), 8.12 (d, J=7.0 Hz, 1 H), 7.79 (d, J=8.5 Hz 1 H), 7.57 (t, J=7.8 Hz, 1 H), 7.49 (d, J=8.7 Hz, 1 H), 7.25-7.23 (m, 2 H), 5.54 (s, 2 H), 3.48 (bs, 2 H), 3.28 (bs, 2 H), 2.08 (s, 3 H). LC-MS: exact mass calculated for C₂₄H₂₂N₆O₂: 426.18, found 427.20 (M+1 )⁺.

5 -(2 -(3 -((Cyclopentylamino)methyl)phenyl)-1 H-pyrrolo[2,3 -b]pyridin-4 -yl)-1 H-indazol-3 -amine (5 n). The crude solid was purified by column chromatography (1 % Et₃ N and 20 % MeOH in EtOAc) to give the titled compound as a brown solid (12.5 mg, 8 %). ¹H NMR (400 MHz, DMSO-d₆ ) δ 12.20 (br s, 1 H), 8.28-8.21 (m, 2 H), 7.94 (s, 1 H), 7.84 (d, J=7.47 Hz, 1 H), 7.72 (d, J=8.79 Hz, 1 H), 7.44-7.37 (m, 2 H), 7.32 (d, J=7.47 Hz, 1 H), 7.21-7.15 (m, 2 H), 5.56 (s, 2 H), 3.74 (s, 2 H), 3.04-3.01 (m, 1 H), 1.74-1.72 (m, 2 H), 1.65-1.64 (m, 2 H), 1.46 -1.45 (m, 2 H), 1.38-1.36 (m, 2 H). ¹³ C NMR (100 MHz, DMSO-d₆ ) δ 151.02, 150.43, 147.82, 143.71, 141.65, 141.75, 139.06, 131.98, 129.18, 128.35, 127.27, 125.70, 124.17, 120.91, 119.11, 115.07, 115.05, 110.53, 109.70, 97.35, 59.11, 52.20, 33.02, 24.18 . HRMS (ESI): exact mass calculated for C₂₆H₂₆N₆ : 422.2292, found 423.2289 (M+1 )⁺.

5 -(2 -(3 -Isobutoxyphenyl)-1 H-pyrrolo[2,3 -b]pyridin-4 -yl)-1 H-indazol-3 -amine (5 o). The crude residue was purified by column chromatography (90 % EtOAc in petroleum ether) to afford the titled compound as an off-white solid (28 mg, 19 %). ¹H NMR (DMSO-d6): 6 12.19 (br s, 1 H), 11.55 (br s, 1 H), 8.26 (d, J=4.8 Hz, 1 H), 8.24 (s, 1 H), 7.72 (dd, J=1.6, 8.8 Hz, 1 H), 7.57-7.54 (m, 2 H), 7.39-7.37 (m, 2 H), 7.20-7.18 (m, 2 H), 6.91-6.89 (m, 1 H), 5.54 (br s, 2 H), 3.85 (d, J=6.4 Hz, 2 H), 2.06-2.04 (m, 1 H), 1.02 (d, J=6.8 Hz, 6 H). HRMS (ESI): exact mass calculated for C₂₄H₂₃N₅O: 397.1975, found 398.1970 (M+1 )⁺.

N-(3 -(4 -(3 -Amino-1 H-indazol-5 -yl)-1 H-pyrrolo(2,3 -b]pyridin-2 -yl)phenyl)-4 -methylbenzensulfonamide (5 t). The crude residue was purified by column chromatography (10 % MeOH in EtOAc) and triturated with Et₂O to afford the titled compound as an off-white solid (53 mg, 29 %). ¹H NMR (DMSO-d₆ ): δ 12.25 (br s, 1 H), 11.58 (br s, 1 H), 10.32 (s, 1 H), 8.27 (d, J=5.2 Hz, 1 H), 8.22 (s, 1 H), 7.69-7.66 (m, 3 H), 7.61-7.59 (m, 2 H), 7.42 (d, J=8.4 Hz, 1 H), 7.32 (t, J=8.0 Hz, 3 H), 7.18 (d, J=4.8 Hz, 1 H), 7.05 (dd, J=1.2, 8.0 Hz, 1 H), 6.92 (d, J=2.0 Hz, 1 H), 5.57 (br s, 2 H), 3.33 (s, 3 H). HRMS (ESI): exact mass calculated for C₂H₂₂ NeO₂ S: 494.1598, found 495.1592 (M+1 )⁺.

5 -(2 -(3 -((Phenylamino)methyl)phenyl)-1 H-pyrrolo[2,3 -b]pyridin-4 -yl)-1 H-indazol-3 -amine (5 p). The crude residue was purified by column chromatography (90 % EtOAc in petroleum ether) followed by HPLC to give the titled compound as a yellow solid (25 mg, 16 %).

¹H NMR (400 MHz, DMSO-d₆ ) δ 12.22 (br s, 1 H), 8.26 (d, J=5.27 Hz, 1 H), 7.85 (d, J=7.47 Hz, 1 H), 7.71 (d, J=9.67 Hz, 1 H), 7.41 (d, J=7.91 Hz, 2 H), 7.36-7.32 (m, 1 H), 7.19 (d, J=4.83 Hz, 1 H), 7.16 (d, J=1.32 Hz, 1 H), 7.04 (t, J=7.69 Hz, 3 H), 6.61 (d, J=7.91 Hz, 3 H), 6.54 -6.48 (m, 1 H), 6.26-6.20 (m, 1 H), 5.56 (s, 2 H), 4.31 (d, J=5.71 Hz, 2 H). HRMS (ESI): exact mass calculated for C₂₇H₂₂N₆ : 430.199, found 431.1978 (M+1 )⁺.

5 -(2 -(2 -(Benzyloxy)phenyl)-1 H-pyrrolo[2,3 -b]pyridin-4 -yl)-1 H-indazol-3 -amine (5 q). The crude solid was purified by column chromatography (10 % MeOH in EtOAc) and triturated with Et₂O to afford the titled compound as an off-white solid (46 mg, 29 %). ¹H NMR (DMSO-d₆ ): δ 11.85 (br s, 1 H), 11.54 (br s, 1 H), 8.24 (d, J=4.8 Hz, 1 H), 8.16 (s, 1 H), 7.92 (dd, J=1.6, 7.6 Hz, 1 H), 7.57 (dd, J=1.6, 8.8 Hz, 1 H), 7.53-7.52 (m, 2 H), 7.35-7.30 (m, 1 H), 7.27-7.24 (m, 6 H), 7.22 (d, J=4.8 Hz, 1 H), 7.11-6.98 (m, 1 H), 5.50 (br s, 2 H), 5.24 (s, 2 H). HRMS (ESI): exact mass calculated for C₂₇H₂₁N₅O: 431.1719, found 432.1814 (M+1 )⁺.

(3 -(4 -(3 -Amino-1 H-indazol-5 -yl)-1 H-pyrrolo[2,3 -b]pyridin-2 -yl)phenyl)(4 -methylpiperazin-1 -y/)methanone (5 v). The resulting solid was washed with hot DCM to afford the titled compound as a brown solid (125 mg, 75 %). ¹H NMR (DMSO-d₆ ): δ 12.29 (br s, 1 H), 11.59 (br s, 1 H), 8.28 (d, J=4.8 Hz, 1 H), 8.26 (s, 1 H), 8.06-8.03 (m, 1 H), 8.00 (s, 1 H), 7.73 -7.70 (m, 1 H), 7.54 (t, J=7.8 Hz, 1 H), 7.40-7.38 (m, 1 H), 7.28 (d, J=2.0 Hz, 1 H), 7.21 (d, J=5.2 Hz, 1 H), 5.58 (s, 2 H), 3.65-3.62 (m, 2 H), 2.41-2.38 (m, 2 H), 2.30-2.27 (m, 2 H), 2.20 (s, 3 H). HRMS (ESI): exact mass calculated for C₂₆H₂₅N_{70: 451.2193}, found 452.2191 (M+1 )⁺.

5 -(2 -(3 -(Pyridin-2 -ylmethoxy)phenyl)-1 H-pyrrolo[2,3 -b]pyridin-4 -yl)-1 H-indazol-3 -amine (5 w). The crude residue was purified by column chromatography (5 % MeOH in EtOAc) to obtain the titled compound as beige solid (58 mg, 36 %). ¹H NMR (400 MHz, DMSO-d₆ ) δ 12.24 (s, 1 H), 11.57 (s, 1 H), 8.60 (d, J=4.3 Hz, 1 H), 8.26 (d, J=5.0 Hz, 1 H), 8.24 (s, 1 H), 7.88 -7.84 (m, 1 H), 7.75-7.69 (m, 2 H), 7.62-7.57 (m, 2 H), 7.44-7.32 (m, 3 H), 7.22 (d, J=1.9 Hz, 1 H), 7.20 (d, J=4.9 Hz, 1 H), 7.02 (d, J=8.0 Hz, 1 H), 5.57 (s, 2 H), 5.29 (s, 2 H). HRMS (ESI): exact mass calculated for C₂₆H₂₀N₆O: 432.1777, found 433.1767 (M+1 )⁺.

5 -(2 -(3 -((Tetrahydro-2 H-pyran-4 -yl)methoxy)phenyl)-1 H-pyrrolo[2,3 -b]pyridin-4 -yl)-1 H-indazol-3 -amine (5×). The crude solid was purified by column chromatography (10 % MeOH in EtOAc) to obtain the titled compound as white solid (26 mg, 16 %). ¹H NMR (400 MHz, DMSO-d₆ ) δ 12.22 (s, 1 H), 11.57 (s, 1 H), 8.26 (d, J=5.0 Hz, 1 H), 8.24 (s, 1 H), 7.72 (dd, J=8.8, 1.2 Hz, 1 H), 7.58-7.56 (m, 2 H), 7.40 (d, J=8.8 Hz, 1 H), 7.38-7.34 (m, 1 H), 7.21-7.18 (m, 2 H), 6.92 (d, J=7.1 Hz, 1 H), 5.56 (s, 2 H), 3.94 (d, J=6.4 Hz, 2 H), 3.90 (dd, J=11.5, 3.4 Hz, 2 H), 3.42-3.34 (m, 2 H), 2.12-1.95 (m, 1 H), 1.72 (d, J=11.2 Hz, 2 H), 1.41-1.32 (m, 2 H). LC-MS: exact mass calculated for C₂₆H₂₅N₅O₂: 439.20, found 440.3 (M+1 )⁺.

5 -(2 -(3 -(2 -Morpholinoethoxy)phenyl)-1 H-pyrrolo[2,3 -b]pyridin-4 -yl)-1 H-indazol-3 -amine (5 y). The crude solid was purified by column chromatography (10 % MeOH in EtOAc) to obtain the titled compound as white solid (123 mg, 73 %). ¹H NMR (400 MHz, DMSO-d₆ ) δ 12.20 (s, 1 H), 11.57 (s, 1 H), 8.26 (d, J=5.0 Hz, 1 H), 8.24 (s, 1 H), 7.73 (d, J=8.8 Hz, 1 H), 7.59-7.56 (m, 2 H), 7.40 (d, J=8.4 Hz, 1 H), 7.38-7.34 (m, 1 H), 7.22 (d, J=2.1 Hz, 1 H), 7.20 (d, J=4.9 Hz, 1 H), 6.92 (d, J=8.4 Hz, 1 H), 5.56 (s, 2 H), 4.19 (t, J=5.7 Hz, 2 H), 3.63-3.56 (m, 4 H), 2.73 (t, J=5.7 Hz, 2 H), 2.48-2.51 (m, 4 H). LC-MS: exact mass calculated for C₂₆H₂₆N₆O₂: 454.21, found 455.3 (M+1 )⁺.

5 -(2 -(3 -(Benzyloxy)phenyl)-1 H-pyrrolo[2,3 -b]pyridin-4 -yl)-1 H-indazol-3 -amine (5 r). The crude residue was purified by column chromatography (90 % EtOAc in petroleum ether) to afford the titled compound as an off-white solid (92 mg, 58 %). ¹H NMR (500 MHz, DMSO-d₆ ) δ 12.23 (s, 1 H), 11.59 (s, 1 H), 8.31-8.25 (m, 2 H), 7.75 (dd, J=8.6, 1.6 Hz, 1 H), 7.71 (t, J=2.0 Hz, 1 H), 7.61 (d, J=7.8 Hz, 1 H), 7.52 (d, J=7.5 Hz, 2 H), 7.41 (dt, J=14.4, 8.0 Hz, 4 H), 7.35 (t, J=7.4 Hz, 1 H), 7.28-7.18 (m, 2 H), 7.01 (dd, J=8.2, 2.4 Hz, 1 H), 5.58 (s, 2 H), 5.22 (s, 2 H). ¹³ C NMR (126 MHz, DMSO-d₆ ) δ 159.35, 150.94, 143.82, 141.71, 138.56, 137.53, 133.50, 130.46, 128.36, 127.20, 120.86, 118.9, 114.99, 112.13, 110.47, 97.79, 69.86 . LC-MS: exact mass calculated for C₂₇H₂₁N₅O: 431.17, found 432.3 (M+1 )⁺.

5 -(2 -(3 -((4 -Methoxybenzyl)oxy)phenyl)-1 H-pyrrolo[2,3 -b]pyridin-4 -yl)-1 H-indazol-3 -amine (5 z). The crude residue was purified by recrystallization using MeOH/DCM and hexane to afford the titled compound as an off-white solid (109 mg, 64 %). ¹H NMR (DMSO-d₆ ): δ 12.19 (br s, 1 H), 11.55 (br s, 1 H), 8.26 (d, J=5.2 Hz, 1 H), 8.24 (s, 1 H), 7.72 (dd, J=1.2, 8.4 Hz, 1 H), 7.66 (s, 1 H), 7.58 (d, J=8.0 Hz, 1 H), 7.39-7.34 (m, 4 H), 7.20-7.16 (m, 2 H), 6.98-6.95 (m, 3 H), 5.54 (br s, 2 H), 5.11 (s, 2 H), 3.75 (s, 3 H). HRMS (ESI): exact mass calculated for C₂₈H₂₃N₅O₂: 461.1925 found 462.1922 (M+1 )⁺.

5 -(2 -(3 -(Morpholinosulfonyl)phenyl)-1 H-pyrrolo[2,3 -b]pyridin-4 -yl)-1 H-indazol-3 -amine (5 aa). The resulting solid was suspended in EtOAc and filtered through a pad of celite and concentrated under reduced pressure to afford the titled compound as a pale-yellow solid (17.5 mg, 10 %). 1 H NMR (DMSO-d₆ ): δ 12.53 (br s, 1 H), 11.58 (br s, 1 H), 8.32-8.28 (m, 2 H), 8.26 (s, 1 H), 7.74-7.69 (m, 4 H), 7.41 (d, J=8.8 Hz, 1 H), 7.34 (s, 1 H), 7.23 (d, J=4.8 Hz, 1 H), 5.55 (br s, 2 H), 3.65-3.61 (m, 4 H), 2.96-2.92 (m, 4 H). HRMS (ESI): exact mass for C₂₄H₂₂N₆O₃ S: 474.1547, found 475.1548 (M+1 )⁺.

5 -(2 -(4 -Benzyloxy)phenyl)-1 H-pyrrolo[2,3 -b]pyridin-4 -yl)-1 H-indazol-3 -amine (5 s). The resulting solid was triturated with hexane and Et₂O to afford the titled compound as a pale-yellow solid (75 mg, 47 %). ¹H NMR (DMSO-d₆ ): δ 12.09 (br s, 1 H), 11.53 (br s, 1 H), 8.22-8.18 (m, 2 H), 7.92 (d, J=8.8 Hz, 1 H), 7.71 (d, J=1.6, 8.8 Hz, 1 H), 7.49-7.43 (m, 2 H), 7.41-7.37(m, 3 H), 7.34-7.30 (m, 1 H), 7.18 (d, J=4.8 Hz, 1 H), 7.12 (d, J=8.8 Hz, 2 H), 7.07 (d, J=2.4 Hz, 1 H), 5.53 (br s, 2 H), 5.18 (s, 2 H). HRMS (ESI): exact mass calculated for C₂₇H₂₁N₅O: 431.1719, found 432.1813 (M+1 )⁺.

N-(4 -(4 -(3 -Amino-1 H-indazol-5 -yl)-1 H-pyrrolo(2,3 -b]pyridin-2 -yl)phenyl)-4 -methylbenzene sulfonamide (5 u). The crude residue was purified by column chromatography (10 % MeOH in EtOAc) and triturated with Et₂O to afford the titled compound as an off-white solid (51 mg, 28 %). ¹H NMR (DMSO-d₆ ): δ 12.10 (br s, 1 H), 11.55 (br s, 1 H), 10.40 (br s, 1 H), 8.22 (d, J=4.8 Hz, 1 H), 8.20 (s, 1 H), 7.83 (d, J=8.8 Hz, 2 H), 7.68 (dd, J=2.0, 8.8 Hz, 3 H), 7.37 (t, J=9.2 Hz, 3 H), 7.17 (s, 1 H), 7.15 (d, J=4.0 Hz, 1 H), 7.06 (d, J=2.0 Hz, 1 H), 5.54 (br s, 2 H), 2.32 (s, 3 H). HRMS (ESI): calculated for C₂₇H₂₂N₆O₂ S: 494.1598, found 495.1592 (M+1 )⁺.

### General Procedure for Synthesis of 4 -chloro-2 -(substituted aryl)-1 H-pyrrolo[2, 3 -b]pyridine (17 a-i)

Followed the general procedure for synthesis of compounds 15 a-ac. The crude residue was used in the next step without further purification unless otherwise stated below.

2 -(6 -(Benzyloxy)pyridin-3 -yl)-4 -chloro-1 H-pyrrolo[2,3 -b]pyridine (17 d). The crude residue was purified by column chromatography (70 % EtOAc in petroleum ether) to afford the titled compound as white solid (181 mg, 44 %). ¹H NMR (400 MHz, DMSO-d₆ ) δ 12.55 (s, 1 H), 8.82 (d, J=2.3 Hz, 1 H), 8.33 (dd, J=8.7, 2.3 Hz, 1 H), 8.17 (d, J=5.2 Hz, 1 H), 7.48 (d, J=7.0 Hz, 2 H), 7.42-7.38 (m, 2 H), 7.35 (d, J=7.1 Hz, 1 H), 7.21 (d, J=5.1 Hz, 1 H), 7.05-6.99 (m, 2 H), 5.42 (s, 2 H). LC-MS: exact mass calculated for C₁₉H₁₄³⁵ClN₃O: 335.0825, found 336.1 (M+1 )⁺.

2 -(5 -(Benzyloxy)pyridin-3 -yl)-4 -chloro-1 H-pyrrolo[2,3 -b]pyridine (17 e). The crude residue was suspended in EtOAc and filtered through a pad of celite and evaporated to give the titled product as brown solid (383 mg, 93 %). ¹H NMR (400 MHz, DMSO-d₆ ) δ 12.67 (s, 1 H), 8.84 (d, J=1.6 Hz, 1 H), 8.34 (d, J=2.6 Hz, 1 H), 8.22 (d, J=5.2 Hz, 1 H), 8.11 (dd, J=2.6, 1.6 Hz, 1 H), 7.52 (d, J=7.1 Hz, 2 H), 7.43-7.41 (m, 2 H), 7.39-7.35 (m, 1 H), 7.25-7.24 (m, 2 H), 5.29 (s, 2 H). LC-MS: exact mass calculated for C₁₉H₁₄³⁵ClN₃O: 335.0825, found 336.1 (M+1 )⁺.

2 -(2 -(Benzyloxy)pyridin-4 -yl)-4 -chloro-1 H-pyrrolo[2,3 -b]pyridine (17 g). The crude solid was purified by column chromatography (90 % EtOAc in petroleum ether) to give the titled product as brown solid (371 mg, 90 %). ¹H NMR (400 MHz, DMSO-d₆ ) δ 12.72 (s, 1 H), 8.26 -8.24 (m, 2 H), 7.63 (d, J=5.7 Hz, 1 H), 7.52-7.47 (m, 3 H), 7.42-7.38 (m, 2 H), 7.35 (d, J=7.5 Hz, 1 H), 7.33 (d, J=1.8 Hz, 1 H), 7.26 (d, J=5.3 Hz, 1 H), 5.41 (s, 2 H). LC-MS: exact mass calculated for C₁₉H₁₄₃₅ CINs0: 335.0825, found 336.1 (M+1 ).

General procedure for synthesis of 5 -(2 -substituted aryl-1 H-pyrrolo(2,3 -b]pyridin-4 -yl)-1 H-indazol-3 -amine (6 a-i). Followed the general procedure for synthesis of compounds 5.

5 -(2 -(3 -(Benzyloxy)-5 -fluorophenyl)-1 H-pyrrolo[2,3 -b]pyridin-4 -yl)-1 H-indazol-3 -amine (6 a). The crude solid was recrystallized from EtOAc to afford the titled compound as a beige solid (88 mg, 53 %). ¹H NMR (400 MHz, DMSO-d₅ ) δ 12.27 (s, 1 H), 11.58 (s, 1 H), 8.29 (d, J=4.9 Hz, 1 H), 8.23 (s, 1 H), 7.73 (d, J=8.4 Hz, 1 H), 7.61-7.31 (m, 9 H), 7.22 (d, J=4.9 Hz, 1 H), 6.90 (d, J=10.7 Hz, 1 H), 5.56 (s, 2 H), 5.22 (s, 2 H). LC-MS: exact mass calculated for C₂₇H₂₀FN₅O: 449.1652, found 450.3 (M+1 )+

5 -(2 -(3 -(Benzyloxy)-5 -((2 -methoxyethoxy)methyl)phenyl)-1 H-pyrrolo[2,3 -b]pyridin-4 -yl)-1 H-indazol-3 -amine (6 b). The crude solid was washed with water (3 mL) and hexane (3×3 mL) to afford the titled compound as an off-white solid (146 mg, 76 %). ¹H NMR (400 MHz, DMSO-de) 6 12.23 (s, 1 H), 11.57 (s, 1 H), 8.26 (d, J=4.9 Hz, 1 H), 8.23 (s, 1 H), 7.72 (d, J=8.8 Hz, 1 H), 7.61 (s, 1 H), 7.55 (s, 1 H), 7.51 (d, J=7.5 Hz, 2 H), 7.46-7.38 (m, 3 H), 7.38-7.32 (m, 1 H), 7.22-7.17 (m, 2 H), 6.97 (s, 1 H), 5.57 (s, 2 H), 5.20 (s, 2 H), 4.52 (s, 2 H), 3.60-3.55 (m, 2 H), 3.53-3.46 (m, 2 H), 3.25 (s, 3 H). LC-MS: exact mass calculated for C₃₁H₂₉N₅O₃: 519.2270, found 520.3 (M+1 )⁺

5 -(2 -(3 -(Benzyloxy)-5 -((2 -methoxyethyl)amino)phenyl)-1 H-pyrrolo[2,3 -b]pyridin-4 -yl)-1 H-indazol-3 -amine (6 c). The crude product was purified by HPLC to afford the titled compound as a pale-yellow solid (26 mg, 14 %). ¹H NMR (500 MHz, DMSO-d₅ ) δ 12.23 (s, 1 H), 8.31 (s, 1 H), 8.27 (d, J=4.9 Hz, 1 H), 7.82 (d, J=8.4 Hz, 1 H), 7.49-7.32 (m, 6 H), 7.21 (d, J=4.9 Hz, 1 H), 7.07 (d, J=1.7 Hz, 1 H), 6.90 (s, 1 H), 6.83 (s, 1 H), 5.11 (s, 2 H), 3.52-3.48 (m, 2 H), 3.38-3.25 (m, 7 H). LC-MS: exact mass calculated for C₂₀H₂₆N₆O₂: 504.2274, found 505.30 (M+1 )⁺

5 -(2 -(6 -(Benzyloxy)pyridin-3 -yl)-1 H-pyrrolo[2,3 -b]pyridin-4 -yl)-1 H-indazol-3 -amine (6 d). The crude solid was purified by column chromatography (10 % MeOH in EtOAc) to afford the title compound as a beige solid (43 mg, 27 %). ¹H NMR (400 MHz, DMSO-d₆ ) δ 12.27 (s, 1 H), 11.57 (s, 1 H), 8.81 (d, J=2.3 Hz, 1 H), 8.32 (dd, J=8.4, 2.3 Hz, 1 H), 8.25 (d, J=5.0 Hz, 1 H), 8.23 (s, 1 H), 7.72 (d, J=8.9 Hz, 1 H), 7.50-7.46 (m, 2 H), 7.42-7.36 (m, 3 H), 7.34 (d, J=7.4 Hz, 1 H), 7.21-7.19 (m, 2 H), 7.01 (d, J=8.9 Hz, 1 H), 5.56 (s, 2 H), 5.42 (s, 2 H). HRMS (ESI): exact mass calculated for C₂₆H₂₀N₆O: 432.1699, found 433.1777 (M+1 )⁺.

5 -(2 -(5 -(Benzyloxy)pyridin-3 -yl)-1 H-pyrrolo[2,3 -b]pyridin-4 -yl)-1 H-indazol-3 -amine (6 e). The crude solid was purified by column chromatography (1 % Et₃ N and 10 % MeOH in EtOAc) to afford the titled compound as a beige solid (86 mg, 54 %) ¹H NMR (400 MHz, DMSO-d₆ ) δ 12.37 (s, 1 H), 11.58 (s, 1 H), 8.85 (d, J=1.6 Hz, 1 H), 8.34-8.27 (m, 2 H), 8.24 (s, 1 H), 8.08 (s, 1 H), 7.74 (d, J=8.6 Hz, 1 H), 7.52 (d, J=7.5 Hz, 2 H), 7.47-7.35 (m, 5 H), 7.23 (d, J=5.0 Hz, 1 H), 5.57 (s, 2 H), 5.28 (s, 2 H). HRMS (ESI): exact mass calculated for C₂₆H₂₀N₆O: 432.1699, found 433.1777 (M+1 )⁺.

5 -(2 -(2 -Fluoro-6 -((phenylamino)methy)pyridin-4 -yl)-1 H-pyrrolo[2,3 -b]pyridin-4 -yl)-1 H-indazol-3 -amine (6 f). The crude product was purified by HPLC to afford the titled compound as a pale-yellow solid (65 mg, 39 %). ¹H NMR (500 MHz, DMSO-d6) 5 12.38 (s, 1 H), 8.49 (t, J=5.6 Hz, 1 H), 8.35 (d, J=5.6 Hz, 1 H), 8.29 (s, 1 H), 7.80 (d, J=8.4 Hz, 1 H), 7.51 (d, J=8.5 Hz, 1 H), 7.49 (d, J=8.5 Hz, 1 H), 7.26 (d, J=8.5 Hz, 1 H), 7.17 (s, 1 H), 7.07 (t, J=8.5 Hz, 1 H), 6.62 -6.56 (m, 3 H), 4.37 (s, 2 H). LR-MS: exact mass calculated for C₂₆H₂₀FN₇: 449.1764, found 450.20 (M+1 )⁺.

5 -(2 -(2 -(Benzyloxy)pyridin-4 -yl)-1 H-pyrrolo[2,3 -b]pyridin-4 -yl)-1 H-indazol-3 -amine (6 g). The crude product was purified by column chromatography (10 % MeOH in EtOAc) to afford the titled compound as white solid (78 mg, 49 %). ¹H NMR (400 MHz, DMSO-d₆ ) δ 12.43 (s, 1 H), 11.58 (s, 1 H), 8.33 (d, J=5.0 Hz, 1 H), 8.27-8.20 (m, 2 H), 7.73 (dd, J=8.7, 1.6 Hz, 1 H), 7.62 (dd, J=5.4, 1.6 Hz, 1 H), 7.53-7.45 (m, 4 H), 7.42-7.38 (m, 3 H), 7.34 (d, J=7.3 Hz, 1 H), 7.25 (d, J=5.0 Hz, 1 H), 5.57 (s, 2 H), 5.41 (s, 2 H). HRMS (ESI): exact mass calculated for C₂₆H₂₀N₆O: 432.1699, found 433.1777 (M+1 )⁺.

5 -(2 -(2 -(Benzylthio)pyridin-4 -yl)-1 H-pyrrolo[2,3 -b]pyridin-4 -yl)-1 H-indazol-3 -amine (6 h). The crude solid was purified by HPLC to afford the titled compound as a yellow solid (33 mg, 20 %). ¹H NMR (500 MHz, DMSO-d) 5 12.51 (s, 1 H), 8.51 (d, J=5.3 Hz, 1 H), 8.36 (d, J=4.9 Hz, 1 H), 8.32 (d, J=1.5 Hz, 1 H) 7.92 (s, 1 H), 7.85-7.80 (m, 1 H), 7.72 (dd, J=5.3, 1.6 Hz, 1 H), 7.50 (d, J=2.2 Hz, 1 H), 7.48 (d, J=8.7 Hz, 1 H), 7.46-7.43 (m, 2 H), 7.33-7.29 (m, 2 H), 7.27 (d, J=5.0 Hz, 1 H), 7.26-7.21 (m, 1 H), 4.50 (s, 2 H). LR-MS: exact mass calculated for C₂₆H₂₀N₆ S: 448.1470, found 449.2 (M+1 )⁺.

5 -(2 -(2 -(Benzylthio)-6 -fluoropyridin-4 -yl)-1 H-pyrrolo[2,3 -b]pyridin-4 -yl)-1 H-indazol-3 -amine (6 i). The crude solid was purified by HPLC to afford the titled compound as yellow (29 mg, 17 %). ¹H NMR (500 MHz, DMSO-d₆ ) δ 12.56 (s, 1 H), 8.40 (d, J=4.9 Hz, 1 H), 8.33 (s, 1 H), 7.91 (s, 1 H), 7.84 (dd, J=8.7, 1.7 Hz, 1 H), 7.61 (d, J=1.8 Hz, 1 H), 7.51 (d, J=2.5 Hz, 3 H), 7.50-7.46 (m, 2 H), 7.34 (t, J=7.5 Hz, 2 H), 7.31-7.23 (m, 2 H), 4.47 (s, 2 H). LR-MS: exact mass calculated for C₂₆H₁₉FN₆ S: 466.1376, found 467.1 (M+1 )⁺.

Synthesis of 4 -(benzyloxy)-2 -bromopyridine (20 a). Compound 19 a (0.436 g, 0.303 mL, 2.5 mmol) was added to a mixture of compound 18 a (0.376 g, 2.16 mmol) and cesium carbonate (1.037 g, 3.182 mmol) in DMF (5 mL) under argon at 0° C. The reaction mixture was allowed to warm to rt and stirred for 4 h. The reaction mixture was poured onto ice/water (20 mL) and extracted with EtOAc (3×50 mL). The combined organic layers was washed with brine (3×20 mL), dried over anhydrous sodium sulfate, concentrated under reduced pressure and purified by column chromatography (50 % EtOAc in petroleum ether) to afford the titled compound as a light brown solid (466 mg, 82 %). ¹H NMR (400 MHz, DMSO-d6) 6 8.20 (d, J=5.8 Hz, 1 H), 7.54-7.34 (m, 6 H), 7.11 (dd, J=5.8, 2.3 Hz, 1 H), 5.24 (s, 2 H). LR-MS: exact mass calculated for C₁₂H₁₀ ⁷⁹BrNO: 262.9946, found 264.0 (M+1 )⁺.

General procedure for synthesis of 4 -bromo-2 -(aryloxy)pyridine (20 b,c). Potassium tert-butoxide (0.58 g, 1.36 mmol) in anhydrous THF (10 mL)was added to a solution of compound 18 b (0.2 g, 1.136 mmol) in anhydrous THF (5 mL) at 0° C. Compounds 19 b,c (1.36 mmol) were then added, and the resulting mixtures allowed to warm to rt and stirred for 4 h. The reaction mixtures were poured onto ice/water (20 mL) and extracted with EtOAc (3×50 mL). The combined organic layers was washed with brine (3×20 mL), dried over anhydrous sodium sulfate and concentrated under reduced pressure. The crude residue was used in the next step without further purification unless otherwise stated below.

4 -Bromo-2 -(pyridin-4 -ylmethoxy)pyridine (20 b). The crude residue was purified by column chromatography (50 % EtOAc in petroleum ether) to afford the titled compound as a light brown oil (240 mg, 80 %). ¹H NMR (500 MHz, DMSO-d₆ ) δ 8.56 (ddd, J=4.8, 1.8, 0.9 Hz, 1 H), 8.07 (d, J=5.4 Hz, 1 H), 7.81 (td, J=7.7, 1.8 Hz, 1 H), 7.45 (dt, J=7.9, 1.0 Hz, 1 H), 7.33 (ddd, J=7.6, 4.9, 1.2 Hz, 1 H), 7.29-7.24 (m, 2 H), 5.44 (s, 2 H). LR-MS: exact mass calculated for C₁₁H₉⁷⁹BrN₂O: 263.9898, found 265.0 (M+1 )⁺.

General procedure for synthesis of 2 -(substituted aryl)-4,4,5,5 -tetramethyl-1,3,2 -dioxaborolane (21 a-c). A suspension of 20 a-c (6.3747 mmol), [1,1 ′-bis(diphenylphosphino)ferrocene]dichloropalladium(II) (0.238 g, 0.325 mmol), potassium acetate (1.918 g, 19.541 mmol), and bis(pinacolato)diboron (2.15 g, 8.466 mmol) in dioxane (9 mL) and water (1 mL) under argon was allowed to stir at 110° C. for 18 h. The reaction was cooled and diluted with EtOAc (30 mL), washed with water (3×20 mL). The organic layer was dried over anhydrous sodium sulfate and concentrated under reduced pressure. The crude residue was used in the next step without further purification unless otherwise stated below.

2 -(Pyridin-4 -ylmethoxy)-4 -(4,4,5,5 -tetramethyl-1,3,2 -dioxaborolan-2 -yl)pyridine (21 b). The crude residue was purified by column chromatography on (70 % EtOAc in petroleum ether) to afford the titled compound as a brown solid (978 mg, 49 %). ¹H NMR (500 MHz, DMSO-d6) 5 8.64 (s, 2 H), 8.18 (dd, J=5.0, 0.8 Hz, 1 H), 7.45 (s, 2 H), 7.17 (dd, J=5.0, 0.8 Hz, 1 H), 7.11 (s, 1 H), 5.43 (s, 2 H), 1.30 (s, 12 H). LR-MS: exact mass calculated for C₁₇H₂₁BN₂O₃ : 312.1645, found 313.0 (M+1 )⁺.

2 -Phenethoxy-4 -(4,4,5,5 -tetramethyl-1,3,2 -dioxaborolan-2 -yl)pyridine (21 c). The crude residue was purified by column chromatography (50 % EtOAc in petroleum ether) to afford the titled compound as a brown solid (832 mg, 40 %). ¹H NMR (500 MHz, DMSO-d₆ ) δ 8.19 (dd, J=4.9, 0.9 Hz, 1 H), 7.31 (d, J=4.9 Hz, 4 H), 7.22 (d, J=4.3 Hz, 1 H), 7.12 (dd, J=4.9, 0.9 Hz, 1 H), 6.91 (d, J=1.0 Hz, 1 H), 4.46 (t, J=6.8 Hz, 2 H), 3.02 (t, J=6.8 Hz, 2 H), 1.30 (s, 12 H). LR-MS: exact mass calculated for C₁₉H₂₄BNO₃: 325.1849, found 326.1 (M+1 )⁺.

Synthesis of 4 -chloro-1 -(methoxymethyl)-1 H pyrrolo[2,3 -b]pyridine (23 b). Methoxymethyl chloride (3.1659 g, 39.33 mmol)was added to a solution of 4 -chloro-1 H-pyrrolo[2,3 -b]pyridine (22, 5.0 g, 32.77 mmol) and potassium carbonate (6.793 g, 49.15 mmol) in DMF (20 mL) at 0° C. The reaction mixture was allowed to warm to rt and stirred for 18 h. The reaction was poured onto ice/water and extracted with EtOAc (3×50 mL). The combined organic layers were washed with brine (3×20 mL), dried over anhydrous sodium sulfate and concentrated under reduced pressure. The crude residue was purified by column chromatography (90 % EtOAc in petroleum ether) to afford the titled compound as a beige solid (4.62 g, 72 %). ¹H NMR (500 MHz, DMSO-d₆ ) δ 8.26 (d, J=5.2 Hz, 1 H), 7.78 (d, J=3.6 Hz, 1 H), 7.29 (d, J=5.2 Hz, 1 H), 6.61 (d, J=3.6 Hz, 1 H), 5.62 (s, 2 H), 3.22 (s, 3 H). LR-MS: exact mass calculated for CH₉³⁵ClN₂0: 196.0403, found 197.3 (M+1 )⁺.

Synthesis of tert-butyl 4 -chloro-1 H-pyrrolo[2,3 -b]pyridine-1 -carboxylate (23 a). Di-tert-butyl dicarbonate (20.70 g, 21.81 mL, 94.85 mmol) was added to a solution of 4 -chloro-1 H-pyrrolo[2,3 -b]pyridine (22, 9.6 g, 62.92 mmol) and 4 -dimethylaminopyridine (11.59 g, 94.85 mmol) in DMF (10 mL) under argon at rt. The reaction mixture was stirred at rt for 18 h. The reaction was extracted between EtOAc and water. The combined organic layers were washed with brine (3×20 mL), dried over anhydrous sodium sulfate and concentrated under reduced pressure. The crude residue was purified by column chromatography (50 % EtOAc in petroleum ether) to afford the titled product as a beige solid (15.69 g, 99 %). ¹H NMR (500 MHz, DMSO-d₆ ) δ 8.36 (d, J=5.2 Hz, 1 H), 7.89 (d, J=4.0 Hz, 1 H), 7.44 (dd, J=5.2, 1.1 Hz, 1 H), 6.74 (dd, J=4.1, 1.2 Hz, 1 H), 1.62 (s, 9 H). LR-MS: exact mass calculated for C₁₂H₁₃³⁵ClN₂02: 252.0666, found 253.3 (M+1 )⁺.

General procedure for synthesis of N-Protected-4 -chloro-2 -iodo-1 H-pyrrolo[2,3 -b]pyridine (24 a,b). n-Butyllithium (8.7 mL, 8.70 mmol, 1 M solution in THF) was slowly added to a solution of compounds 23 a,b (7.253 mmol) in anhydrous THF at −78° C. under argon. The reaction mixture was allowed to stir for 30 minutes at 0° C. Iodine (1.0125 g, 7.978 mmol) in THF (10 mL) was slowly added at −78° C. The reaction mixture was allowed to warm to rt and stirred for 2 h. The reaction was poured onto ice/H₂O and extracted with EtOAc (3×50 mL). The combined organic layers were washed with saturated ammonium chloride (20 mL), water (20 mL) and brine (20 mL). The organic layer was dried over anhydrous sodium sulfate and removed under reduced pressure.

4 -Chloro-2 -iodo-1 -(methoxymethyl)-1 H-pyrrolo[2,3 -b]pyridine (24 b). The crude residue was purified by column chromatography (90 % EtOAc in petroleum ether) to afford the titled compound as a light brown solid (1 g, 43 %). ¹H NMR (500 MHz, DMSO-d₆ ) δ 8.21 (d, J=5.2 Hz, 1 H), 7.29 (d, J=5.2 Hz, 1 H), 7.01 (s, 1 H), 5.61 (s, 2 H), 3.23 (s, 3 H). LR-MS: exact mass calculated for C₉H₈ ³⁵ ClIN₂O: 321.9370, found 323.0 (M+1 )’.

tert-Butyl 4 -chloro-2 -iodo-1 H-pyrrolo[2,3 -b]pyridine-1 -carboxylate (24 a). The crude solid was purified by column chromatography (50 % EtOAc in petroleum ether) to afford the titled compound as a light brown solid (1.8 g, 65 %). ¹H NMR (500 MHz, DMSO-d₆ ) δ 8.19 (t, J=1.0 Hz, 1 H), 7.53 (t, J=1.4 Hz, 1 H), 7.05 (dd, J=1.7, 0.9 Hz, 1 H), 1.58 (s, 9 H). LR-MS: exact mass calculated for C₁₂H₁₂₃₅ CIlN₂02: 377.9632, found 379.4 (M+1 )⁺.

General procedure for synthesis of N-Protected-4 -chloro-2 -(substituted aryl)-1 H-pyrrolo[2,3 -b]pyridine (25 a-c). A mixture of compounds 21 a-c (3.139 mmol), compounds 24 a,b (2.64 mmol), bis(triphenylphosphine)palladium(II) dichloride (0.0926 g, 0.132 mmol) and cesium carbonate (2.58 g, 7.9 mmol) was suspended in dioxane (9 mL) and water (1 mL) under argon. The reaction mixture was stirred at 80° C. for 18 h. The reaction was cooled, diluted with EtOAc and washed with water (3×20 mL). The organic layer was dried over anhydrous sodium sulfate and concentrated under reduced pressure. The crude residue was used in the next step without further purification unless otherwise stated below.

2 -(4 -(Benzyloxy)pyridin-2 -yl)-4 -chloro-1 -(methoxymethyl)-1 H-pyrrolo[2,3 -b]pyridine (25 a). The crude residue was purified by column chromatography (1 % Et₃ N and 50 % EtOAc in petroleum ether) to afford the titled compound as a brown solid (990 mg, 99 %).¹ H NMR (500 MHz, DMSO-d₆ ) δ 8.54 (d, J=5.7 Hz, 1 H), 8.34 (d, J=5.2 Hz, 1 H), 7.73 (d, J=2.4 Hz, 1 H), 7.54-7.48 (m, 2 H), 7.44 (td, J=7.3, 6.3, 1.5 Hz, 2 H), 7.38 (dd, J=8.0, 6.2 Hz, 2 H), 7.25 (s, 1 H), 7.10 (dd, J=5.8, 2.4 Hz, 1 H), 6.21 (s, 2 H), 5.33 (s, 2 H), 3.08 (s, 3 H). LR-MS: exact mass calculated for C₂₁H₁₈³⁵ClN₃O₂ : 379.1088, found 380.3 (M+1 )⁺.

General procedure for synthesis of 5 -(2 -substituted aryl-1 H-pyrrolo(2,3 -b]pyridin-4 -yl)-1 H-indazol-3 -amine (6 j-I). A mixture of compounds 25 a-c (2.373 mmol), compound 9 (0.0676 g, 0.261 mmol), [1,1 ′-bis(di-tert-butylphosphino)ferrocene]dichloropalladium(II) catalyst (0.0154 g, 0.0236 mmol) and cesium carbonate (0.232 g, 0.712 mmol) was suspended in dioxane (9 mL) and water (1 mL). The reaction mixture was stirred at 110° C. for 18 h. The reaction was cooled, diluted with EtOAc and washed with water (3×20 mL). The organic layer was dried over anhydrous sodium sulfate and concentrated under reduced pressure. The crude residue was then taken up in concentrated HCl/MeOH (4:1, 5 mL) for 6 j or TBAF (5 mL, 1 M solution in THF) for 6 k,l and refluxed for 8 h. The reaction mixture was cooled and concentrated under reduced pressure.

5 -(2 -(4 -(Benzyloxy)pyridin-2 -yl)-1 H-pyrrolo[2,3 -b]pyridin-4 -yl)-1 H-indazol-3 -amine (6 j). The crude residue was purified by HPLC to afford the titled compound as an orange solid (35 mg, 31 %) 1 H NMR (500 MHz, DMSO-d₆ ) δ 12.60 (s, 2 H), 8.57 (d, J=5.9 Hz, 1 H), 8.41 (d, J=5.1 Hz, 1 H), 8.35 (d, J=1.5 Hz, 1 H), 7.95 (d, J=2.4 Hz, 1 H), 7.85 (dd, J=8.7, 1.7 Hz, 1 H), 7.63 (s, 1 H), 7.57-7.51 (m, 4 H), 7.48-7.44 (m, 2 H), 7.43-7.38 (m, 1 H), 7.32 (d, J=5.0 Hz, 1 H), 7.17 (dd, J=6.2, 2.4 Hz, 1 H), 5.37 (s, 2 H). LR-MS: exact mass calculated for C₂₆H₂₀N₆O: 432.1699, found 433.3 (M+1 )⁺.

5 -(2 -(2 -(Pyridin-4 -ylmethoxy)pyridin-4 -yl)-1 H-pyrrolo[2,3 -b]pyridin-4 -yl)-1 H-indazol-3 -amine (6 k). The crude residue was purified by HPLC to afford the titled compound as a bright yellow solid (11 mg, 10 %). ¹H NMR (500 MHz, DMSO-d₆ ) δ 12.60 (s, 1 H), 8.83 (d, J=5.7 Hz, 2 H), 8.43-8.38 (m, 2 H), 8.21 (d, J=5.5 Hz, 1 H), 7.92-7.88 (m, 3 H), 7.69-7.65 (m, 2 H), 7.54 (d, J=7.5 Hz, 2 H), 7.31 (d, J=5.0 Hz, 1 H), 5.69 (s, 2 H). LR-MS: exact mass calculated for C₂₅H₁ NO: 433.1651, found 434.3 (M+1 )⁺.

5 -(2 -(2 -Phenethoxypyridin-4 -yl)-1 H-pyrrolo[2,3 -b]pyridin-4 -yl)-1 H-indazol-3 -amine (61 ). The crude residue was purified by HPLC to afford the titled compound as a bright yellow solid
(13 mg, 11 %). ¹H NMR (400 MHz, DMSO-d₆ ) δ 12.56-12.13 (m, 1 H), 11.58 (s, 1 H), 8.34 (d, J=5.0 Hz, 1 H), 8.27-8.23 (m, 1 H), 8.22 (d, J=5.5 Hz, 1 H), 7.74 (dd, J=8.7, 1.6 Hz, 1 H), 7.59 (dd, J=5.4, 1.5 Hz, 1 H), 7.47 (d, J=1.9 Hz, 1 H), 7.44-7.39 (m, 2 H), 7.37-7.30 (m, 4 H), 7.25 (dd, J=5.6, 2.1 Hz, 2 H), 5.56 (s, 2 H), 4.54 (t, J=6.9 Hz, 2 H), 3.08 (t, J=6.8 Hz, 2 H). LR-MS: exact mass calculated for C₂₇H₂₂N₆O: 446.1855, found 446.3 (M+1 )⁺.

### Pharmacokinetic Studies

Pharmacokinetic studies were contracted to Sygnature Discovery Limited. Intrinsic clearance and half life were determined using cryopreserved pooled mouse CD1 hepatocytes. Test compounds were analysed at 1 μM in Williams Media E buffer (0.01 % DMSO) at a cell density of 0.5×10⁶ cells mL⁻¹ . Cells were incubated at 37° C. for one hour with shaking and compound depletion was measured by LC-MS/MS at six timepoints (0.25, 5, 10, 20, 40, and 60 minutes).

In vivo pharmacokinetic studies were carried out in CD1 mice in triplicate intravenously at a dose of 1 mg kg-¹ and intraperitoneally at a dose of 10 mg kg-1 . Prior formulation/solubility assessment was performed in routine excipients e.g., DMSO: cyclodextrin or HMPC/Tween 80 . Intravenous time points were taken at 5 min, 15 min, 30 min, 1, 2, 4, 8, and 24 hours and intraperitoneal time points were taken at 15 min, 30 min, 1, 2, 4, 6, 8, and 24 hours. Blood samples were prepared by protein precipitation with methanol or acetonitrile containing internal standard and then measured quantitative bioanalysis by LC-MS/MS.

### REFERENCES

1 . Gamble, C., et al., Inhibitory kappa B Kinases as targets for pharmacological regulation. British journal of pharmacology, 2012. 165 (4 ): p. 802-819.
2 . Cohen, M.S., et al., Structural bioinformatics-based design of selective, irreversible kinase inhibitors. Science, 2005. 308 (5726 ): p. 1318-1321.
3 . Paul, A., et al., Inhibitory-κB kinase (IKK) α and nuclear factor-κB (NFκB)-inducing kinase (NIK) as anti-cancer drug targets. Cells, 2018. 7 (10 ): p. 176.
4 . Liu, S., et al., Crystal structure of a human IκB kinase 0 asymmetric dimer. Journal of Biological Chemistry, 2013. 288 (31 ): p. 22758-22767.
5 . Polley, S., et al., Structural Basis for the Activation of IKK1 /α. Cell reports, 2016. 17 (8 ): p. 1907-1914.
6 . Anthony, N. G., et al., Inhibitory kappa B kinase a (IKKα) inhibitors that recapitulate their selectivity in cells against isoform-related biomarkers. Journal of medicinal chemistry, 2017. 60 (16 ): p. 7043-7066.
7 . Christopher John Andrew, J.D.K., Lackey Karen Elizabeth, LH-indazole-3 -amine compounds as IKK1 inhibitors, S.B.C. Glaxo group LTD, Editor. 2008.
8 . Ishiyama, T., et al., A Stoichiometric Aromatic CQ H Borylation Catalyzed by Iridium (i)/2, 2 ′-Bipyridine Complexes at Room Temperature. Angewandte Chemie International Edition, 2002. 41 (16 ): p. 3056-3058.
9 . Miyaura, N., K. Yamada, and A. Suzuki, A new stereospecific cross-coupling by the palladium-catalyzed reaction of 1 -alkenylboranes with 1 -alkenyl or 1 -alkynyl halides. Tetrahedron Letters, 1979. 20 (36 ): p. 3437-3440.
10 . D′Alterio, M. C., et al., Mechanistic Aspects of the Palladium-Catalyzed Suzuki-Miyaura Cross-Coupling Reaction. Chemistry-A European Journal, 2021. 27 (54 ): p. 13481-13493.
11 . Ishiyama, T., M. Murata, and N. Miyaura, Palladium (0 )-catalyzed cross-coupling reaction of alkoxydiboron with haloarenes: a direct procedure for arylboronic esters. The Journal of Organic Chemistry, 1995. 60 (23 ): p. 7508-7510.

### Additional Experimental Section

General Methods. Unless otherwise stated, commercially available materials were used without further purification. Air- or moisture-sensitive reactions were carried out under a nitrogen atmosphere. Anhydrous solvents were obtained from Sigma-Aldrich. Flash chromatography was performed using silica gel under standard techniques¹ (Acros, 60 A, 35-70 μm) or using a Biotage SP4 automated chromatography system (SNAP KP-Sil, 60 A, 40-63 μm cartridges; detection wavelength: 254 nm; monitoring: 280 nm). NMR spectra (¹ H and ¹³ C) were recorded on either a JEOL ECX-400 (400 MHz); Bruker Avance3 /DPX400 (400 MHz) or Bruker Avance/DPX500 (500 MHz) instruments. Chemical shifts (δ) are quoted in parts per million (ppm) relative to an internal solvent reference. Coupling constants (J) are recorded in Hertz. Low resolution mass spectroscopy was carried out on ThermoFinnigan LCQ Duo by direct infusion, high resolution mass spectroscopy was carried out on a Exactive (thermo scientific) LCMS mass spectrometer. Reverse phase HPLC purifications were conducted on a Water HPLC system comprising a Waters 1525 binary HPLC pump, Waters 717 autosampler, Waters 2487 dual λ absorbance detector (254 nm), using a semi-preparative (50×21.2 mm) Luna 5μ C18 column(eluting with an acetonitrile/water gradient with 0.1 % TFA in each solvent using the following gradient;

| Time | Flow rate | | % Acetonitrile 0.1% |
|---|---|---|---|
| (min) | (ml/min) | % Water 0.1% TFA | TFA |
| 0 | 6 | 90 | 10 |
| 25 | 6 | 50 | 50 |
| 30 | 6 | 30 | 70 |
| 35 | 6 | 90 | 10 |
| 40 | 0 | 90 | 10 |

Microwave reactions were carried out using a Biotage Initiator-8 Microwave synthesiser (operating at 2.45 GHz). Thin-layer chromatography (TLC) was carried out on aluminium-backed SiO₂ μlates (Merck, silica gel 60, F₂₅₄ ) and spots visualised using ultra-violet light (254 nm) or by staining with potassium permanganate. All tested compounds were determined to be ≥95 % purity by LC-MS and analytical HPLC unless otherwise stated.

### General Procedures

All commercially available reagents and solvents used were obtained from Sigma-Aldrich, Fluorochem Fisher Scientific, Acros, Alfa Aesar, Apollo scientific and Advanced ChemBlocks and used without further purification. Air- or moisture-sensitive reactions were carried out under argon or nitrogen atmosphere.

Microwave reactions were carried out using a Biotage Initiator system.

Flash chromatography was performed using a Biotage SP4 automated chromatography system using silica stationary phase (Fisher Scientific, 60 A, 35-70 micron; detection wavelength: 254 nm; monitoring: 280 nm) and the mobile phase used are detailed in the text.

Reverse phase HPLC purifications were conducted on Shimadzu Prominance HPLC using a semi-preparative (50×21.2 mm) Luna 5 μm C18 column at 40° C.; flow rate: 6 ml/min; detection wavelength: 254 nm eluting with an acetonitrile/water gradient with 0.1 % TFA.

NMR spectra were recorded on either a Bruker Avance3 /DPX400 (400 MHz), Bruker DRX500 (500 MHz), Bruker AV400 (400 MHz), Bruker AV500 HD (500 MHz) or Bruker AV600 (600 MHz) instrument and analysed using Advanced Chemistry Development Labs (ACD/labs) NMR processor 12.00 or MestReNova 10.0 software. Chemical shifts (δ) are recorded in parts per million (ppm) relative to an internal solvent reference (tetramethylsilane) and coupling constants (J) in Hertz (Hz). Splitting patterns were indicated as singlet (s), broad singlet (br. s), doublet (d), doublet of doublet (dd), triplet (t), quartet (q) and multiplet (m).

LCMS was carried out on an Agilent Technologies 1220 series LC system with Agilent 6100 series quadrupole mass spectrometer in ESI/APCI mode. Separation was achieved with an Agilent Eclipse C18 4.6×50 mm column; flow rate:1 ml/min; detection:254 nm; sample volume:10 μl; mobile phase: acetonitrile/5 mM ammonium acetate:water/5 mM ammonium acetate; 5 %, 1.48 min; 5-100 %, 8 min; 100 %, 13.5 min; 100-5 %, 16.5 min; 18 min. HRMS was carried out on an Exactive (Thermo scientific) or LTQ orbitrap (Thermo scientific).

### Arylalkyl Substituents at Position 2 of the Aminopyridine Ring

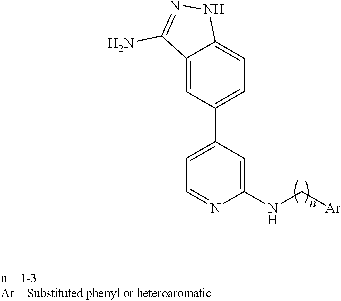

### 5 -[2 -(Benzylamino)pyridin-4 -vyl]-1 H-indazol-3 -amine SU1067

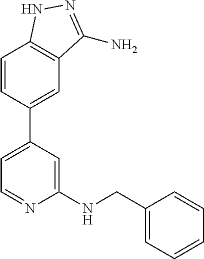 [SMILES: Nc1n[nH]c2ccc(-c3ccnc(NCc4ccccc4)c3)cc12]

A suspension of N-benzyl-4 -chloropyridin-2 -amine (0.104 g, 0.48 mmol), 2 -fluoro-5 -(4,4,5,5 -tetramethyl-1,3,2 -dioxaborolan-2 -yl)benzonitrile (0.164 g, 0.66 mmol), [1,1 ′-bis(di-tert-butylphosphino)ferrocene]dichloropalladium(II) catalyst (0.029 g, 0.04 mmol) in IPA/H₂O (3:1.5 mL) was degassed with nitrogen. t-Butylamine (0.25 mL, 2.38 mmol) was then added and degassed using nitrogen. The reaction was placed in a microwave and irradiated at 160° C. for 40 min. Once cooled to room temperature the reaction mixture was concentrated under reduced pressure the suspended in EtOH (4.5 mL). To this suspension was added hydrazine hydrate (0.20 mL, 4.00 mmol) and the reaction mixture was placed in a microwave and irradiated at 165° C. for 30 min. The reaction mixture was diluted with EtOAc and concentrated under reduced pressure. Purification of the resulting solid was carried out using column chromatography (100 % Hexane to 100 % EtOAc). The resulting solid was triturated with Et₂O and filtered under reduced pressure to give the title compound as a pale yellow solid (0.051 g, 0.16 mmol). ¹H NMR (400 MHz, DMSO-d₆ ): δ 4.54 (d, J=6.0 Hz, 2 H), 5.48 (br s, 2 H), 6.78 -6.80 (m, 2 H), 7.09 (t, J=6.0, Hz, 1 H), 7.20-7.23 (m, 1 H), 7.29-7.37 (m, 5 H), 7.50 (d, J=8.8 Hz, 1 H), 7.99 (d, J=5.2 Hz, 1 H), 8.06 (s, 1 H), 11.52 (br s, 1 H). HRMS: For C₁₉H₁₈N₅ requires 316.1557 found 316.1553 . (M+H)⁺

### 3 -(((4 -(3 -Amino-1 H-indazol-5 -yl)pyridin-2 -yl)amino)methyl)benzonitrile SU1220

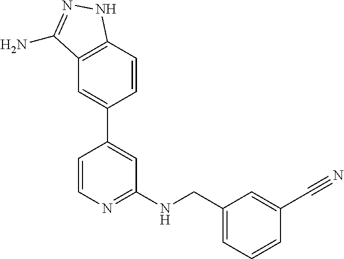 [SMILES: N#Cc1cccc(CNc2cc(-c3ccc4[nH]nc(N)c4c3)ccn2)c1]

4 -(((4 -Bromopyridin-2 -yl)amino)methyl)benzonitrile (0.101 g, 0.35 mmol, 1 eq), 5 -(4,4,5,5 -tetramethyl-1,3,2 -dixaboralan-2 -yl)-1 H-indazol-3 -amine (0.136 g, 0.53 mmol, 1.5 eq), [1,1 ′-bis(di-tert-butylphosphino)ferrocene]dichloropalladium(II) catalyst (0.011 g, 17.5 μmol, 0.05 eq) and potassium phosphate (0.186 g, 0.88 mmol, 2.5 eq) were dissolved in ethanol (0.75 ml) and water (0.75 ml) and heated at 100° C. overnight. The solvent was removed at reduced pressure and the residue purified by preparative HPLC to give the desired compound (64 mg, 0.12 mmol, 40 %) as a yellow powder ¹H NMR (500 MHz, DMSO) δ_{H} 8.83 (br s, 1 H), 8.32 (s, 1 H), 8.04 (d, J=6.7 Hz, 1 H), 7.90 (d, J=1.6 Hz, 1 H), 7.79 (d, J=7.8 Hz, 1 H), 7.76 (d, J=7.8 Hz, 1 H), 7.67 (dd, J=8.8 and 1.6 Hz, 1 H), 7.61 (dd, J=7.8 and 7.8 Hz, 1 H), 7.38 (d, J=8.8 Hz, 1 H), 7.25 (s, 1 H), 7.22 (d, J=6.7 Hz, 1 H), 4.73 (s, 1 H); ³ C NMR (125 MHz, CDCl₃ ) δ_{c} 150.5, 142.4, 132.8, 132.8, 131.8, 131.5, 131.5, 130.4, 125.7, 125.2, 125.1, 121.3, 119.2, 119.2, 115.1, 112.0, 112.0, 111.0, 111.0 45.7.

### 5 -(2 -((3 -Methoxybenzyl)-amino)pyridin-4 -yl)-1 H-indazol-3 -amine SU1222

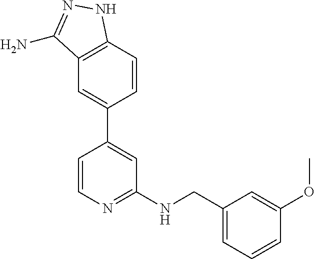 [SMILES: COc1cccc(CNc2cc(-c3ccc4[nH]nc(N)c4c3)ccn2)c1]

4 -Bromo-N-(3 -methoxybenzyl)pyridin-2 -amine (0.101 g, 0.35 mmol, 1 eq), 5 -(4,4,5,5 -tetramethyl-1,3,2 -dixaboralan-2 -yl)-1 H-indazol-3 -amine (0.136 g, 0.525 mmol, 1.5 eq), [1,1 ′-bis(di-tert-butylphosphino)ferrocene]dichloropalladium(II) catalyst (0.011 mg, 17.5 μmol, 0.05 eq) and potassium phosphate (0.186 g, 0.875 mmol, 2.5 eq) were dissolved in ethanol (0.75 ml) and water (0.75 ml) and heated at 100° C. overnight. The solvent was removed at reduced pressure and the residue purified by preparative HPLC to give the desired compound (0.092 g, 0.20 mmol, 57 %) as a yellow powder. ¹H NMR (500 MHz, DMSO)^{5H} 8.95 (br s, 1 H), 8.34 (s, 1 H), 8.04 (d, J=6.8 Hz, 1 H), 7.68 (d, J=8.3 Hz, 1 H), 7.39 (d, J=8.8 Hz, 1 H), 7.33-7.30 (m, 2 H), 7.23 (d, J=6.8 Hz, 1 H), 7.02-6.99 (m, 2 H), 6.89 (dd, J=8.3 and 2.1 Hz, 1 H), 4.64 (s, 2 H), 3.76 (s, 3 H); ¹³ C NMR (125 MHz, DMSO) oc 160.0, 158.7, 153.6, 150.6, 142.4, 139.0, 130.3, 130.3, 125.7, 125.0, 121.4, 120.1, 120.1, 115.1, 113.9, 113.5, 111.0, 110.8, 55.6.

### 2 -(3 -(((4 -(3 -Amino-1 H-indazol-5 -yl)pyridin-2 -yl)amino)methyl)phenyl)propan-2 -ol SU1232

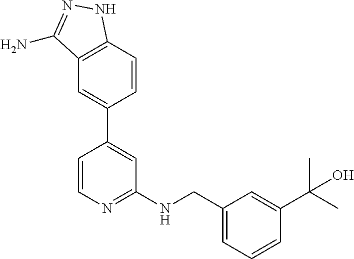 [SMILES: CC(C)(O)c1cccc(CNc2cc(-c3ccc4[nH]nc(N)c4c3)ccn2)c1]

4 -Bromo-N-(3 -methoxybenzyl)pyridin-2 -amine (0.112 g, 0.35 mmol, 1 eq), 5 -(4,4,5,5 -tetramethyl-1,3,2 -dixaboralan-2 -yl)-1 H-indazol-3 -amine (0.136 g, 0.525 mmol, 1.5 eq), [1,1 ′-bis(di-tert-butylphosphino)ferrocene]dichloropalladium(II) catalyst (0.011 g, 17.5 μmol, 0.05 eq) and potassium phosphate (0.186 g, 0.875 mmol, 2.5 eq) were dissolved in ethanol (0.75 ml) and water (0.75 ml) and heated at 100° C. overnight. The solvent was removed at reduced pressure and the residue purified by preparative HPLC to give the desired compound (0.057 g, 0.12 mmol, 33 %) as a yellow powder. ⁴ H NMR (500 MHz, DMSO) 5_{H} 8.98 (br s, 1 H), 8.35 (d, J=1.2 Hz, 1 H), 8.04 (d, J=6.8 Hz, 1 H), 7.69 (dd, J=8.8 and 1.7 Hz, 1 H), 7.57 (s, 1 H), 7.42 -7.39 (m, 2 H), 7.35-7.32 (m, 2 H), 7.25-7.22 (m, 2 H), 4.66 (s, 2 H), 1.43 (s, 6 H); ¹³ C NMR (125 MHz, CDCl₃ ) δ_{c} 153.4, 151.6, 150.4, 142.5, 138.8, 136.5, 128.7, 128.7, 125.8, 125.8, 125.6, 125.0, 124.5, 124.5, 121.4, 115.0, 111.1, 110.7, 71.1, 45.9, 32.5.

### 5 -(2 -((4 -(Trifluoromethyl)benzyl)amino)pyidin-4 -yl)-1 H-indazol-3 -amine SU1095

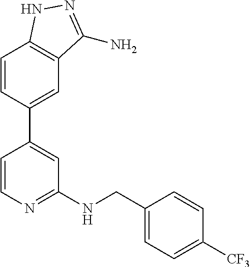 [SMILES: Nc1n[nH]c2ccc(-c3ccnc(NCc4ccc(C(F)(F)F)cc4)c3)cc12]

A suspension of 4 -chloro-N-(4 -(trifluoromethyl)benzyl)pyridine-2 -amine (0.101 g, 0.35 mmol), 2 -fluoro-5 -(4,4,5,5 -tetramethyl-1,3,2 -dioxaborolan-2 -yl)benzonitrile (0.147 g, 0.59 mmol), [1,1 ′-bis(di-tert-butylphosphino)ferrocene]dichloropalladium(II) catalyst (0.029 g, 0.04 mmol) in IPA/H₂O (3:1.5 mL) was degassed with nitrogen. t-Butylamine (0.20 mL, 1.90 mmol) was then added and degassed using nitrogen. The reaction was placed in a microwave and irradiated at 160° C. for 40 min. The reaction was diluted with EtOAc and concentrated under reduced pressure. EtOH (4 mL) was added followed by hydrazine hydrate (0.2 mL, 4.0 mmol) and the mixture irradiated at 165° C. for 30 min. The reaction mixture was then concentrated under reduced pressure and purified using column chromatography (100 % EtOAc—20 % MeOH/EtOAc). The resulting solid was recrystalised from Et₂O and hexane, filtered and dried to give the title compound as an off-white solid (0.050 g, 0.13 mmol, 37 %). ¹H NMR (400 MHz, DMSO-d₆ ): δ 4.63 (d, J=6.0 Hz, 2 H), 5.48 (br s, 2 H), 6.80-6.82 (m, 2 H), 7.23 (t, J=6.2, Hz, 1 H), 7.30 (d, J=8.8 Hz, 1 H), 7.51 (dd, J=1.6, 8.8 Hz, 1 H), 7.56 (d, J=8.0 Hz, 2 H), 7.68 (d, J=8.4 Hz, 2 H), 7.98 (d, J=6.0 Hz, 1 H), 8.07 (s, 1 H), 11.52 (br s, 1 H). HRMS: For C₂₀H₁₇N₅ F₃ requires 384.1431 found 384.1425 . (M+H)⁺

### 4 -(((4 -(3 -Amino-1 H-indazol-5 -yl)pyridin-2 -yl)amino)methyl)benzonitrile SU1212

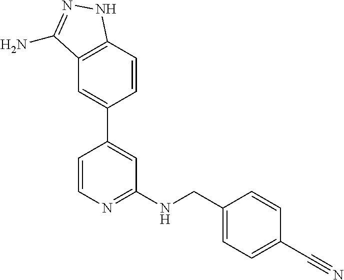 [SMILES: N#Cc1ccc(CNc2cc(-c3ccc4[nH]nc(N)c4c3)ccn2)cc1]

4 -(((4 -Bromopyridin-2 -yl)amino)methyl)benzonitrile (0.432 g, 1.5 mmol, 1 eq), 5 -(4,4,5,5 -tetramethyl-1,3,2 -dixaboralan-2 -yl)-1 H-indazol-3 -amine (0.584 mg, 2.25 mmol, 1.5 eq), [1,1 ′-bis(di-tert-butylphosphino)ferrocene]dichloropalladium(II) catalyst (0.049 mg, 75 μmol, 0.05 eq) and potassium phosphate (0.796 g, 3.75 mmol, 2.5 eq) were dissolved in ethanol (3 ml) and water (3 ml) and heated at 100° C. overnight. The solvent was removed at reduced pressure and the residue purified by preparative HPLC to give the desired compound (0.446 g, 0.98 mmol, 65 %) as a yellow powder (LC-MS purity=99 %). ¹H NMR (500 MHz, DMSO) δ_{H} 9.00 (br s, 1 H), 8.35 (d, J=1.6 Hz, 1 H), 8.05 (d, J=6.7 Hz, 1 H), 7.87 (d, J=8.3 Hz, 2 H), 7.71 (dd, J=8.8 and 1.6 Hz, 1 H), 7.61 (d, J=8.3 Hz, 1 H), 7.40 (d, J=8.8 Hz, 1 H), 7.28 (s, 1 H), 7.24 (d, J=6.7 Hz, 1 H), 4.80 (s, 1 H); ¹³ C NMR (125 MHz, DMSO) δ_{C} 159.0, 1537, 150.3, 143.5, 142.5, 133.1, 128.7, 126.1, 125.2, 121.5, 119.2, 115.0, 111.1, 111.1, 110.8, 45.0.

### 5 -(2 -((4 -(Tert-butyl)benzyl)amino)pyridin-4 -yl)-1 H-indazol-3 -amine SU1114

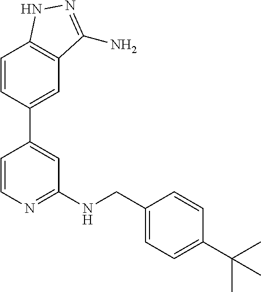 [SMILES: CC(C)(C)c1ccc(CNc2cc(-c3ccc4[nH]nc(N)c4c3)ccn2)cc1]

A suspension of 4 -bromo-N-(4 -tert-butyl)benzyl)pyridine-2 -amine (0.087 g, 0.27 mmol), 2 -fluoro-5 -(4,4,5,5 -tetramethyl-1,3,2 -dioxaborolan-2 -yl)benzonitrile (0.117 g, 0.47 mmol), [1,1 ′-bis(di-tert-butylphosphino)ferrocene]dichloropalladium(II) catalyst (0.017 g, 0.03 mmol) in IPA/H₂O (3:1.5 mL) was degassed with nitrogen. t-Butylamine (0.15 mL, 1.43 mmol) was then added and degassed using nitrogen. The reaction was placed in a microwave and irradiated at 160° C. for 40 min. The reaction was diluted with EtOAc and concentrated under reduced pressure. EtOH (4 mL) was added followed by hydrazine hydrate (0.2 mL, 4.00 mmol) and the mixture irradiated at 165° C. for 30 min. The reaction mixture was then concentrated under reduced pressure and purified using column chromatography (100 % EtOAc—20 % Methanol/EtOAc). The resulting solid was recrystallised from methanol and hexane, filtered and dried to give the title compound as an off-white solid (0.018 g, 0.048 mmol, 17 %). ¹ H NMR (400 MHz, DMSO-d₆ ): δ 1.26 (s, 9 H), 4.49 (d, J=6.0 Hz, 2 H), 5.45 (br s, 2 H), 6.77 (s, 1 H), 6.78 (dd, J=1.6, 5.6 Hz, 1 H), 6.99 (t, J=6.0, Hz, 1 H), 7.27-7.34 (m, 5 H), 7.50 (dd, J=2.0, 8.8 Hz, 1 H), 7.99 (d, J=5.6 Hz, 1 H), 8.05 (s, 1 H), 11.49 (br s, 1 H). HRMS: For C₂₃H₂₆N₅ requires 372.2183 found 372.2182 . (M+H)⁺

### 2 -(4 -(((4 -(3 -Amino-1 H-indazol-5 -yl)pyridin-2 -yl)amino)methyl)phenyl)propan-2 -ol SU1229

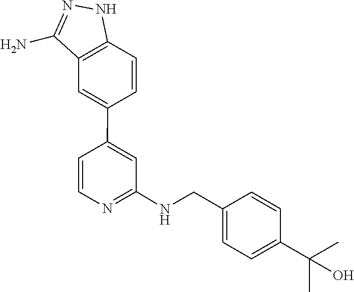 [SMILES: CC(C)(O)c1ccc(CNc2cc(-c3ccc4[nH]nc(N)c4c3)ccn2)cc1]

4 -Bromo-N-(3 -methoxybenzyl)pyridin-2 -amine (0.112 g, 0.35 mmol, 1 eq), 5 -(4,4,5,5 -tetramethyl-1,3,2 -dixaboralan-2 -yl)-1 H-indazol-3 -amine (0.136 g, 0.525 mmol, 1.5 eq), [1,1 ′-bis(di-tert-butylphosphino)ferrocene]dichloropalladium(II) catalyst (0.011 mg, 17.5 μmol, 0.05 eq) and potassium phosphate (0.186 g, 0.875 mmol, 2.5 eq) were dissolved in ethanol (0.75 ml) and water (0.75 ml) and heated at 100° C. overnight. The solvent was removed at reduced pressure and the residue purified by preparative HPLC to give the desired compound (0.092 g, 0.20 mmol 57 %) as a yellow powder. ¹H NMR (500 MHz, DMSO) δ_{H} 9.00 (br s, 1 H), 8.36 (s, 1 H), 8.04 (d, J=6.8 Hz, 1 H), 7.69 (d, J=8.9 Hz, 1 H), 7.49 (d, J=8.3 Hz, 2 H), 7.41 (d, J=8.8 Hz 1 H), 7.35 (d, J=8.3 Hz, 2 H), 7.31 (d, J=1.4 Hz 1 H), 7.22 (dd, J=6.8 and 1.4 Hz, 1 H), 4.63 (s, 2 H), 1.41 (s, 6 H); ¹³ C NMR (125 MHz, CDCl₃ ) δ_{c} 159.0, 158.7, 154.5, 153.4, 150.7, 150.4, 142.5, 134.5, 127.6, 125.9, 125.4, 125.1, 121.4, 115.0, 111.1, 110.7, 71.0, 45.4, 31.4.

### 5 -(2 -((4 -(Methylsulfonyl)benzyl)amino)pyridin-4 -yl)-1 H-indazol-3 -amine SU1221

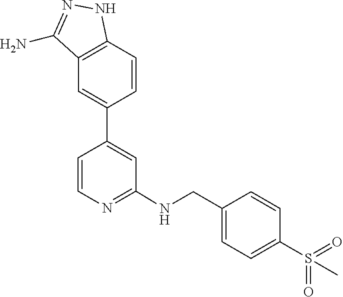 [SMILES: CS(=O)(=O)c1ccc(CNc2cc(-c3ccc4[nH]nc(N)c4c3)ccn2)cc1]

4 -Bromo-N-(4 -(methylsulfonyl)benzyl)pyridin-2 -amine (0.119 g, 0.35 mmol, 1 eq), 5 -(4,4,5,5 -tetramethyl-1,3,2 -dixaboralan-2 -yl)-1 H-indazol-3 -amine (0.136 g, 0.525 mmol, 1.5 eq), [1,1 ′-bis(di-tert-butylphosphino)ferrocene]dichloropalladium(II) catalyst (0.011 g, 17.5 μmol, 0.05 eq) and potassium phosphate (186 mg, 0.875 mmol, 2.5 eq) were dissolved in ethanol (0.75 ml) and water (0.75 ml) and heated at 100° C. overnight. The solvent was removed at reduced pressure and the residue purified by preparative HPLC to give the desired compound (105 mg, 0.21 mmol, 59 %) as a yellow powder. ¹H NMR (500 MHz, DMSO) 5_{H} 9.02 (br s, 1 H), 8.35 (d, J=1.5 Hz, 1 H), 8.06 (d, J=6.7 Hz, 1 H), 7.96 (d, J=8.4 Hz, 2 H), 7.71 (dd, J=8.8 and 1.7 Hz, 1 H), 7.68 (d, J=8.4 Hz, 2 H), 7.41 (dd, J=8.8, 1 H), 7.30 (d, J=1.7, 1 H), 7.24 (dd, J=6.7 and 1.5 Hz, 1 H), 4.82 (s, 2 H), 3.21 (s, 3 H); ¹³ C NMR (125 MHz, DMSO) δ_{C} 158.5, 153.2, 149.8, 143.2, 141.9, 140.0, 128.1, 127.3, 125.5, 124.7, 120.9, 114.5, 110.6, 110.5. 44.4, 43.5.

### 5 -(2 -((Furan-3 -ylmethyl)amino)pyridine-4 -yl)-1 H-indazol-3 -amine SU1111

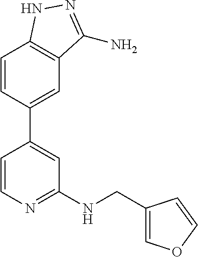 [SMILES: Nc1n[nH]c2ccc(-c3ccnc(NCc4ccoc4)c3)cc12]

A suspension of 4 -bromo-N-(furan-3 -ylmethyl)pyridin-2 -amine (0.035 g, 0.14 mmol), 2 -fluoro-5 -(4,4,5,5 -tetramethyl-1,3,2 -dioxaborolan-2 -yl)benzonitrile (0.065 g, 0.26 mmol), [1,1 ′-bis(di-tert-butylphosphino)ferrocene]dichloropalladium(II) catalyst (0.020 g, 0.03 mmol) in IPA/H₂O (3:1.5 mL) was degassed with nitrogen. t-Butylamine (0.15 mL, 1.43 mmol) was then added and degassed using nitrogen. The reaction was placed in a microwave and irradiated at 160° C. for 40 min. The reaction was diluted with EtOAc and concentrated under reduced pressure. EtOH (4 mL) was added followed by hydrazine hydrate (0.2 mL, 4.00 mmol) and the mixture irradiated at 165° C. for 30 min. The reaction mixture was then concentrated under reduced pressure and purified using column chromatography (100 % EtOAc). The resulting solid was recrystallised from methanol and hexane, filtered and dried to give the title compound as an off-white solid (0.020 g, 0.065 mmol, 47.0 %). ¹H NMR (400 MHz, DMSO-d₆ ): δ 4.52 (d, J=5.6 Hz, 2 H), 5.48 (br s, 2 H), 6.27 (d, J=2.8 Hz, 1 H), 6.38 (d, J=2.0 Hz, 1 H), 6.80 (s, 1 H), 6.82 (d, J=5.2 Hz, 1 H), 6.95 (t, J=5.8, Hz, 1 H), 7.31 (d, J=8.8 Hz, 1 H), 7.52 (dd, J=1.2, 8.8 Hz, 1 H), 7.558-7.571 (m, 1 H), 8.03 (d, J=5.2 Hz, 1 H), 8.06 (s, 1 H), 11.52 (br s, 1 H). HRMS: For C₁₇H₁₆ON₅ requires 306.1349 found 306.1348 . (M+H)⁺

### 5 -(2 -((Pyridin-2 -ylmethyl)amino)pyridin-4 -yl)-1 H-indazol-3 -amine SU1241

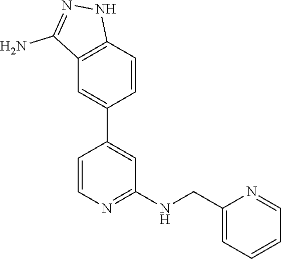 [SMILES: Nc1n[nH]c2ccc(-c3ccnc(NCc4ccccn4)c3)cc12]

4 -Bromo-N-(3 -methoxybenzyl)pyridin-2 -amine (0.092 g, 0.35 mmol, 1 eq), 5 -(4,4,5,5 -tetramethyl-1,3,2 -dixaboralan-2 -yl)-1 H-indazol-3 -amine (0.136 g, 0.525 mmol, 1.5 eq), [1,1 ′-bis(di-tert-butylphosphino)ferrocene]dichloropalladium(II) catalyst (0.011 g, 17.5 μmol, 0.05 eq) and potassium phosphate (0.186 g, 0.875 mmol, 2.5 eq) were dissolved in ethanol (0.75 ml) and water (0.75 ml) and heated at 100° C. overnight. The solvent was removed at reduced pressure and the residue purified by preparative HPLC to give the desired compound (152 mg, 0.28 mmol, 80 %) as a yellow powder. ¹H NMR (500 MHz, DMSO) δ_{H} 8.97 (br s, 1 H), 8.61 (d, J=4.3 Hz, 1 H), 8.38 (s, 1 H), 8.05 (d, J=6.8 Hz, 1 H), 7.90 (ddd, J=7.8, 7.8 and 1.9 Hz, 1 H), 7.74 (d, J=8.9 Hz, 1 H), 7.51 (d, J=7.8 Hz, 1 H), 7.43-7.37 (m, 3 H), 7.23 (dd, J=6.9 and 1.4 Hz, 1 H), 8.83 (s, 2 H); ¹³ C NMR (125 MHz, CDCl₃ ) δ_{c} 156.2, 153.9, 150.1, 149.2, 142.5, 138.4, 137.5, 126.3, 125.5, 123.6, 122.5, 121.5, 120.5, 117.7, 114.9, 111.3, 110.9, 46.6.

### 5 -{2 -[(2 -Phenylethyl)amino]pyridin-4 -y1 }-1 H-indazol-3 -amine SU1092

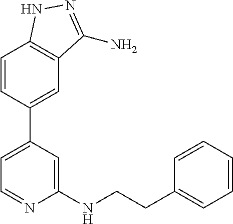 [SMILES: Nc1n[nH]c2ccc(-c3ccnc(NCCc4ccccc4)c3)cc12]

A suspension of 4 -chloro-N-phenethyl-pyridin-2 -amine (0.121 g, 0.52 mmol), 3 -cyano-4 -fluorophenylboronic acid (0.195 g, 0.79 mmol), [1,1 ′-bis(di-tert-butylphosphino)ferrocene]dichloropalladium(II) catalyst (0.032 g, 0.05 mmol) in IPA/H₂O (3:1.5 mL) was degassed with nitrogen. t-Butylamine (0.29 mL, 2.76 mmol) was then added and degassed using nitrogen. The reaction was placed in a microwave and irradiated at 160° C. for 40 min. The reaction was diluted with EtOAc, concentrated under reduced pressure. To this residue was added ethanol (4.5 mL) and hydrazine hydrate (0.25 mL, 5.0 mmol) and the reaction mixture was placed in a microwave and irradiated at 165° C. for 30 min. Reaction mixture was concentrated under reduced pressure and purified by column chromatography (100 % Hexane to 100 % EtOAc) to give the title compound as an off-white solid (0.007 g, 0.021 mmol, 50 %). ¹H NMR (400 MHz, DMSO-d₆ ): s 2.87 (t, J=7.4 Hz, 2 H), 3.52 (q, 2 H, J=6.8 Hz), 6.57 (t, J=5.6 Hz, 1 H), 6.71 (br s, 1 H), 6.79 (dd, J=1.2, 4.4 Hz, 1 H), 7.19-7.22 (m, 1 H), 7.27-7.33 (m, 5 H), 7.53 (dd, 1.6, J=8.8 Hz, , 1 H), 8.02 (d, J=5.2 Hz, 1 H), 8.07 (s, 1 H), 11.51 (br s, 1 H). HRMS: For C₂₀H₂₀N₅ requires 330.1713 found 330.1710 . (M+H)⁺

### 5 -(2 -((2 -(Pyridine-2 -yl)ethyl)amino)pyridine-4 -yl)-1 H-indazol-3 -amine SU1102

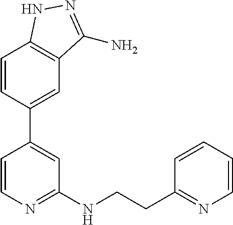 [SMILES: Nc1n[nH]c2ccc(-c3ccnc(NCCc4ccccn4)c3)cc12]

A suspension of 4 -bromo-N-(2 -(pyridine-2 -yl)ethyl)pyridine-2 -amine (0.040 g, 0.14 mmol), 3 -cyano-4 -fluorophenylboronic acid (0.073 g, 0.29 mmol), [1,1 ′-bis(di-tert-butylphosphino)ferrocene]dichloropalladium(II) catalyst (0.021 g, 0.03 mmol) in IPA/H₂O (3:1.5 mL) was degassed with nitrogen. t-Butylamine (0.10 mL, 0.95 mmol) was then added and degassed using nitrogen. The reaction was placed in a microwave and irradiated at 160° C. for 40 min. The reaction was diluted with EtOAc and concentrated under reduced pressure. EtOH (4 mL) was added followed by hydrazine hydrate (0.15 mL, 3.0 mmol) and the mixture irradiated at 165° C. for 30 min. The reaction mixture was then concentrated under reduced pressure and purified using column chromatography (100 % EtOAc—20 % MeOH/EtOAc). The resulting solid was triturated with Et₂O, EtOAc and hexane, filtered and dried to give the title compound as an off-white solid (0.017 g, 0.05 mmol, 35 %). ¹H NMR (400 MHz, DMSO-d₆ ): δ 3.03 (t, J=7.2 Hz, 2 H), 3.65 (q, J=6.5 Hz, 2 H), 5.48 (br s, 2 H), 5.60 (br s, 1 H), 6.71 (s, 1 H), 7.79 (d, J=4.8 Hz, 1 H), 7.22 (q, J=4.1 Hz, 1 H), 7.29 (d, J=3.2 Hz, 1 H), 7.31 (s, 1 H), 7.53 (d, J=8.8 Hz, 1 H), 7.71 (t, J=5.1 Hz, 1 H), 8.02 (d, J=5.2 Hz, 1 H), 8.07 (s, 1 H), 8.52 (d, J=4.0 Hz, 1 H), 11.52 (br s, 1 H). HRMS: For Cj₉H₁₉N₆ requires 331.1666 found 331.1662 . (M+H)⁺

### 5 -(2 -((2 -(Pyridin-3 -yl)ethyl)amino)pyridine-4 -yl)-1 H-indazol-3 -amine SU1121

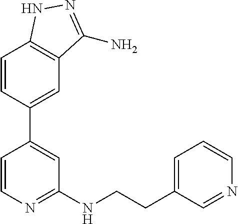 [SMILES: Nc1n[nH]c2ccc(-c3ccnc(NCCc4cccnc4)c3)cc12]

A suspension of 4 -bromo-N-(2 -pyridin-3 -yl)ethyl)pyridine-2 -amine (0.1228 g, 0.44 mmol), 3 -cyano-4 -fluorophenylboronic acid (0.175 g, 0.71 mmol), [1,1 ′-bis(di-tert-butylphosphino)ferrocene]dichloropalladium(II) catalyst (0.028 g, 0.04 mmol) in IPA/H₂O (3:1.5 mL) was degassed with nitrogen. t-Butylamine (0.25 mL, 2.38 mmol) was then added and degassed using nitrogen. The reaction was placed in a microwave and irradiated at 160° C. for 40 min. The reaction was diluted with EtOAc and concentrated under reduced pressure. EtOH (4 mL) was added followed by hydrazine hydrate (0.25 mL, 5.5 mmol) and the mixture irradiated at 165° C. for 30 min. The reaction mixture was then concentrated under reduced pressure and purified using column chromatography (100 % EtOAc—20 % Methanol/EtOAc).

The resulting solid was recrystallised from methanol and hexane, filtered and dried to give the title compound as an off-white solid (0.048 g, 0.15 mmol, 38 %). ¹H NMR (400 MHz, DMSO-d₆ ): δ 2.93 (t, J=7.0 Hz, 2 H), 3.61 (q, J=6.4 Hz, 2 H), 4.62 (br s, 1 H), 5.57 (br s, 1 H), 6.91 (s, 1 H), 6.95 (d, J=5.2 Hz, 1 H), 7.32-7.36 (m, 2 H), 7.58 (d, J=8.8 Hz, 1 H), 7.74 (d, J=7.6 Hz, 1 H), 8.01 (d, J=5.6 Hz, 1 H), 8.17 (s, 1 H), 8.44 (d, J=4.8 Hz, 1 H), 8.52 (s, 1 H), 8.69 (br s, 1 H), 11.62 (br s, 1 H). HRMS: For C₁₉H₁₉N₈ requires 331.1666 found 331.1663 . (M+H)⁺

### 5 -(2 -((2 -Pyridin-4 -yl)ethyl)amino)pyridine-4 -yl)-1 H-indazol-3 -amine SU1123

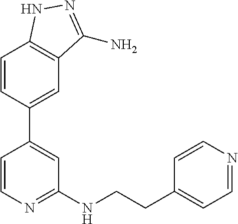 [SMILES: Nc1n[nH]c2ccc(-c3ccnc(NCCc4ccncc4)c3)cc12]

A suspension of 4 -bromo-N-(2 -(pyridine-4 -yl)ethyl)pyridine-2 -amine (0.100 g, 0.36 mmol), 3 -cyano-4 -fluorophenylboronic acid (0.144 g, 0.58 mmol), [1,1 ′-bis(di-tert-butylphosphino)ferrocene]dichloropalladium(II) catalyst (0.027 g, 0.041 mmol) in IPA/H₂O (3:1.5 mL) was degassed with nitrogen. t-Butylamine (0.20 mL, 1.90 mmol) was then added and degassed using nitrogen. The reaction was placed in a microwave and irradiated at 160° C. for 40 min. The reaction was diluted with EtOAc and concentrated under reduced pressure. EtOH (4 mL) was added followed by hydrazine hydrate (0.2 mL, 5.0 mmol) and the mixture irradiated at 165° C. for 30 min. The reaction mixture was then concentrated under reduced pressure and purified using column chromatography (100 % EtOAc—20 % Methanol/EtOAc). The resulting solid was recrystallised from methanol and hexane, filtered and dried to give the title compound as an off-white solid (0.032 g, 0.010 mmol, 27 %). ¹H NMR (400 MHz, DMSO-d₆ ): δ 2.91 (t, J=7.0 Hz, 2 H), 3.95 (q, J=6.4 Hz, 2 H), 5.53 (br s, 2 H), 6.78 (s, 1 H), 6.85 (d, J=4.8 Hz, 1 H), 7.30-7.32 (m, 3 H), 7.54 (dd, J=1.2, 8.8 Hz, 1 H), 8.02 (d, J=5.6 Hz, 1 H), 8.10 (s, 1 H), 8.48 (d, J=6.0 Hz, 2 H), 11.55 (br s, 1 H). HRMS: For C₁₉H₁₉N₆ requires 331.1666 found 331.1661 . (M+H)⁺

### 5 -(2 -((2 -(1 H-Indol-3 -yl)ethyl)amino)pyridine-4 -yl)-1 H-indazol-3 -amine SU1109

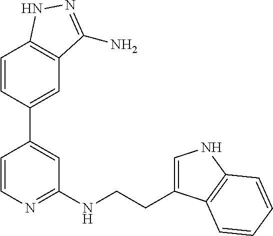 [SMILES: Nc1n[nH]c2ccc(-c3ccnc(NCCc4c[nH]c5ccccc45)c3)cc12]

A suspension of N-(2 -(1 H)-indol-3 -yl)ethyl)-4 -bromopyridin-2 -amine (0.061 g, 0.19 mmol), 3 -cyano-4 -fluorophenylboronic acid (0.086 g, 0.35 mmol), [1,1 ′-bis(di-tert-butylphosphino)ferrocene]dichloropalladium(II) catalyst (0.019 g, 0.03 mmol) in IPA/H₂O (3:1.5 mL) was degassed with nitrogen. t-Butylamine (0.15 mL, 1.42 mmol) was then added and degassed using nitrogen. The reaction was placed in a microwave and irradiated at 160° C. for 40 min. The reaction was diluted with EtOAc and concentrated under reduced pressure. EtOH (4 mL) was added followed by hydrazine hydrate (0.2 mL, 4.0 mmol) and the mixture irradiated at 165° C. for 30 min. The reaction mixture was then concentrated under reduced pressure and purified using column chromatography (100 % EtOAc—20 % MeOH/EtOAc). The resulting solid was recrystallised from methanol and hexane, filtered and dried to give the title compound as an off-white solid (0.028 g, 0.076 mmol, 39.4 %). ¹H NMR (400 MHz, DMSO-d₆ ): δ 2.98 (t, J=7.4 Hz, 2 H), 3.58 (q, J=6.7 Hz, 2 H), 5.48 (br s, 2 H), 6.57 (t, J=5.4 Hz, 1 H), 6.72 (s, 1 H), 6.78 (dd, J=1.2, 5.2 Hz, 1 H), 6.99 (t, J=7.6, 14.8 Hz, 1 H), 7.07 (t, J=7.4 Hz), 7.21 (d, J=2.0 Hz, 1 H), 7.30 (d, J=8.8 Hz, 1 H), 7.34 (d, J=8.0 Hz, 1 H), 7.52 (dd, J=1.6, 8.8 Hz, 1 H), 7.61 (d, J=7.6 Hz, 1 H), 8.03 (d, J=5.2 Hz, 1 H), 8.07 (s, 1 H), 10.82 (br s, 1 H), 11.51 (br s, 1 H).

HRMS: For C₂₂H₂₁N₆ requires 369.1822 found 369.1822 . (M+H)⁺

### 5 -(2 -(4 -Fluorophenethylamino)pyridin-4 -yl)-1 H-indazol-3 -amine SU1150

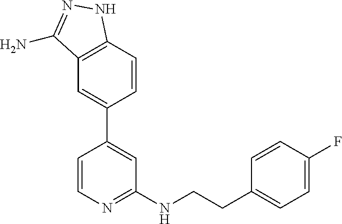 [SMILES: Nc1n[nH]c2ccc(-c3ccnc(NCCc4ccc(F)cc4)c3)cc12]

A suspension of 4 -bromo-N-(4 -fluorophenethyl)pyridin-2 -amine (0.080 g, 0.27 mmol), 3 -cyano-4 -fluorophenylboronic acid pinacol ester (0.135 g, 0.54 mmol), [1,1 ′-bis(di-tert-butylphosphino)ferrocene]dichloropalladium(II) catalyst (0.020 g, 0.03 mmol) in IPA/H₂O (3:1.5 mL) was degassed with nitrogen. t-Butylamine (0.28 mL, 2.72 mmol) was added, and the mixture was heated in the microwave at 160° C. for 45 min. The reaction mixture was concentrated and taken up in EtOH (4 mL) and degassed with nitrogen. Hydrazine hydrate (0.13 mL, 2.70 mmol) was added, and the mixture was heated in the microwave at 165° C. for 30 min. The reaction mixture was concentrated and purified by flash column chromatography (silica gel with EtOAc 50 % in hexane—MeOH 10 % in EtOAc) to afford the title compound as an off-white solid (0.052 g, 0.15 mmol, 55 %). ¹H NMR (400 MHz, DMSO-d₆ ): δ 2.86 (t, Hz, J=7.2 Hz, 2 H), 3.46-3.54 (m, 2 H) 5.47 (br s, 2 H), 6.57 (t, J=5.5 Hz, 1 H), 6.71 (br s, 1 H), 6.78 (dd, J=5.4, 1.4 Hz, 1 H), 7.12 (t, J=8.9 Hz, 1 H), 7.27-7.34 (m, 3 H), 7.75 (dd, J=8.7, 1.6 Hz, 1 H), 8.02 (d, J=5.4 Hz, 1 H) 8.07 (s, 1 H), 11.51 (br s, 1 H).

### 5 -(2 -{[2 -(4 -Chlorophenyl)ethyl]amino}pyridin-4 -yl)-1 H-indazol-3 -amine SU1210

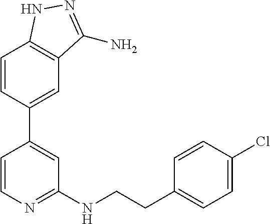 [SMILES: Nc1n[nH]c2ccc(-c3ccnc(NCCc4ccc(Cl)cc4)c3)cc12]

A suspension of 4 -bromo-N-[2 -(4 -chlorophenyl)ethyl]pyridin-2 -amine (0.101 g, 0.33 mmol), 2 -fluoro-5 -(4,4,5,5 -tetramethyl-1,3,2 -dioxaborolan-2 -yl)benzonitrile (0.126 g, 0.49 mmol), [1,1 ′-bis(di-tert-butylphosphino)ferrocene]dichloropalladium(lI) catalyst (0.044 g, 0.04 mmol) in IPA/H₂O (3:1.5 mL) was degassed for 5 min using a steady stream of nitrogen. t-Butylamine (0.20 mL, 2.10 mmol) was added, and the mixture was degassed for 5 min under nitrogen. The reaction mixture was reacted in the microwave at 160° C. for 40 min prior to being cooled to room temperature and concentrated under reduced pressure. The reaction mixture was suspended in EtOH (4.5 mL) to which hydrazine hydrate (0.15 mL, 3.00 mmol) was added prior to being reacted in the microwave at 165° C. for 30 min. Once cooled to room temperature the reaction mixture was concentrated under reduced pressure. Purification by column chromatography (100 % Hexane—50/50 Hexane/EtOAc—100 % EtOAc) followed by trituration with Et₂O and filtration afforded the desired compound as an off white solid (0.037 g, 0.10 mmol, 31.4 %). ¹H NMR (DMSO-d₆ ): δ 2.86 (t, J=14.0 Hz, 2 H), 3.51 (q, J=6.5 Hz, 2 H), 5.45 (br s, 2 H), 6.55 (t, J=10.5 Hz, 1 H), 6.70 (s, 1 H), 6.79 (dd, J=5.5, 1.5 Hz, 1 H), 7.30 (d, J=8.0 Hz, 3 H), 7.35 (d, J=8.5 Hz, 2 H), 7.52 (dd, J=8.5, 1.0 Hz, 1 H), 8.02 (d, J=5.5 Hz, 1 H), 8.06 (s, 1 H), 11.49 (br s, 1 H). HRMS: For C₂₀H₉N₅Cl requires 364.1320, found 364.1323 . (M+H)⁺

### 4 -(2 -((4 -(3 -Amino-1 H-indazol-5 -yl)pyridin-2 -yl)amino)ethyl)phenol SU1110

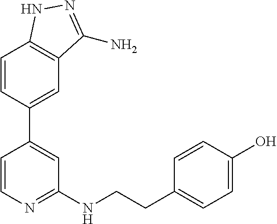 [SMILES: Nc1n[nH]c2ccc(-c3ccnc(NCCc4ccc(O)cc4)c3)cc12]

A suspension of 4 -(2 -((4 -bromopyridin-2 -yl)amino)ethyl)phenol (0.035 g, 0.12 mmol), 3 -cyano-4 -fluorophenylboronic acid (0.048 g, 0.19 mmol), [1,1 ′-bis(di-tert-butylphosphino)ferrocene]dichloropalladium(II) catalyst (0.016 g, 0.03 mmol) in IPA/H₂O (3:1.5 mL) was degassed with nitrogen. t-Butylamine (0.15 mL, 1.43 mmol) was then added and degassed using nitrogen. The reaction was placed in a microwave and irradiated at 160° C. for 40 min. The reaction was diluted with EtOAc and concentrated under reduced pressure. EtOH (4 mL) was added followed by hydrazine hydrate (0.2 mL, 4.0 mmol) and the mixture irradiated at 165° C. for 30 min. The reaction mixture was then concentrated under reduced pressure and purified using column chromatography (100 % EtOAc—20 % MeOH/EtOAc). The resulting solid was recrystallised from methanol and hexane, filtered and dried to give the title compound as an off-white solid (0.0063 g, 0.018 mmol, 15.2 %). ¹H NMR (400 MHz, DMSO-d₆ ): δ 2.74 (t, J=7.4 Hz, 2 H), 3.42-3.47 (m, 2 H), 5.48 (br s, 2 H), 6.51 (t, J=5.6 Hz, 1 H), 6.68 (br s, 2 H), 6.70 (br s, 1 H), 6.79 (d, J=3.2 Hz, 1 H), 7.06 (d, J=8.4 Hz, 2 H), 7.30 (d, J=8.4 Hz, 1 H), 7.52 (d, J=8.8 Hz, 1 H), 8.01 (d, J=5.2 Hz, 1 H), 8.06 (s, 1 H), 9.16 (s, 1 H), 11.51 (br s, 1 H). HRMS: For C₂₀H₂₀ON₅ requires 346.1662 found 346.1662 (M+H)⁺

### 5 -(2 -{[2 -(4 -Methoxyphenyl)ethyl]amino}pyridin-4 -yl)-1 H-indazol-3 -amine SU1207

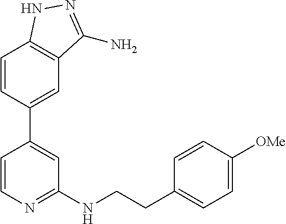 [SMILES: COc1ccc(CCNc2cc(-c3ccc4[nH]nc(N)c4c3)ccn2)cc1]

A suspension of 4 -bromo-N-[2 -(4 -methoxyphenyl)ethyl]pyridin-2 -amine (0.122 g, 0.40 mmol), 2 -fluoro-5 -(4,4,5,5 -tetramethyl-1,3,2 -dioxaborolan-2 -yl)benzonitrile (0.162 g, 0.66 mmol), [1,1 ′-bis(di-tert-butylphosphino)ferrocene]dichloropalladium(II) catalyst (0.030 g, 0.05 mmol) in IPA/H₂O (3:1.5 mL) was degassed for 5 min using a steady stream of nitrogen. t-Butylamine (0.23 mL, 2.19 mmol) was added, and the mixture was degassed for 5 min under nitrogen. The reaction mixture was reacted in the microwave at 160° C. for 40 min prior to being cooled to room temperature and concentrated under reduced pressure. The reaction mixture was suspended in EtOH (4.5 mL) to which hydrazine hydrate (0.15 mL, 3.0 mmol) was added prior to being reacted in the microwave at 165° C. for 30 min. Once cooled to room temperature the reaction mixture was concentrated under reduced pressure. Purification by column chromatography (100 % EtOAc—10 % MeOH/EtOAc) followed by recrystallisation from MeOH (15 mL) and hexane (150 mL) and filtration afforded the desired compound as an off white solid (0.099 g, 0.27 mmol, 68.9 %). ¹H NMR (DMSO-d₆ ): δ 2.81 (t, J=14.8 Hz, 2 H), 3.48 (q, J=6.8 Hz, 2 H), 3.73 (s, 3 H), 5.45 (br s, 2 H), 6.50 (t, J=11.2 Hz, 1 H), 6.71 (s, 1 H), 6.79 (dd, J=5.6, 1.6 Hz, 1 H), 6.88 (dd, J=5.6, 2.0 Hz, 2 H), 7.20 (d, J=8.4 Hz, 2 H), 7.31 (d, J=8.8 Hz, 1 H), 7.53 (1 H, dd, J=8.8, 1.6 Hz, 1 H), 8.03 (d, J=5.6 Hz, 1 H), 8.07 (s, 1 H), 11.49 (br s, 1 H).

HRMS: For C₂₁H₂₂ON₅ requires 360.1812, found 360.1819 . (M+H)⁺

### 5 -(2 -((4 -(tert-Butyl)phenethyl)amino)pyridin-4 -yl)-1 H-indazole-3 -amine SU1206

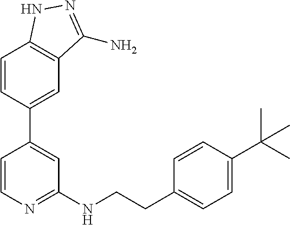 [SMILES: CC(C)(C)c1ccc(CCNc2cc(-c3ccc4[nH]nc(N)c4c3)ccn2)cc1]

A suspension of 4 -bromo-N-[2 -(4 -tert-butylphenyl)ethyl]pyridin-2 -amine (0.105 g, 0.32 mmol), 2 -fluoro-5 -(4,4,5,5 -tetramethyl-1,3,2 -dioxaborolan-2 -yl)benzonitrile (0.129 g, 0.52 mmol), [1,1 ′-bis(di-tert-butylphosphino)ferrocene]dichloropalladium(II) catalyst (0.026 g, 0.040 mmol) in IPA/H₂O (3:1.5 mL) was degassed for 5 min using a steady stream of nitrogen. t-Butylamine (0.25 mL, 2.38 mmol) was added, and the mixture was degassed for 5 min under nitrogen. The reaction mixture was reacted in the microwave at 160° C. for 40 min prior to being cooled to room temperature and concentrated under reduced pressure. The reaction mixture was suspended in EtOH (4.5 mL) to which hydrazine hydrate (0.15 mL, 3.0 mmol) was added prior to being reacted in the microwave at 165° C. for 30 min. Once cooled to room temperature the reaction mixture was concentrated under reduced pressure. Purification by column chromatography (100 % Hexane—50/50 Hexane/EtOAc—100 % EtOAc) followed by recrystallisation from MeOH (15 mL) and hexane (150 mL) and filtration afforded the desired compound as an off white solid (0.080 g, 0.21 mmol, 65.7 %). ¹H NMR (500 MHz, DMSO-d₆ ): δ 1.26 (s, 9 H), 2.83 (t, J=15.0 Hz, 2 H), 3.50 (q, J=6.6 Hz, 2 H), 5.45 (br s, 2 H), 6.53 (t, J=11.0 Hz, 1 H), 6.71 (s, 1 H), 6.79 (dd, J=5.5, 1.5 Hz, 1 H), 7.20 (d, J=8.5 Hz, 2 H), 7.31 (t, J=8.8 Hz, 3 H), 7.52 (dd, J=8.5, 1.5 Hz, 1 H), 8.02 (d, J=5.5 Hz, 1 H), 8.07 (s, 1 H), 11.49 (br s, 1 H).

HRMS: For C₂₄H₂₈N₅ requires 386.2335, found 386.2339 . (M+H)⁺

### 4 -(2 -((4 -(3 -Amino-1 H-indazol-5 -yl)pyridin-2 -yl)amino)ethyl)benzenesulfonamide SU1216

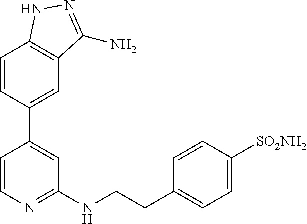 [SMILES: Nc1n[nH]c2ccc(-c3ccnc(NCCc4ccc(S(N)(=O)=O)cc4)c3)cc12]

A suspension of 4 -{2 -[(4 -bromopyridin-2 -yl)amino]ethyl}benzenesulfonamide (0.102 g, 0.29 mmol), 2 -fluoro-5 -(4,4,5,5 -tetramethyl-1,3,2 -dioxaborolan-2 -yl)benzonitrile (0.124 g, 0.51 mmol), [1,1 ′-bis(di-tert-butylphosphino)ferrocene]dichloropalladium(II) catalyst (0.030 g, 0.05 mmol) in IPA/H₂O (3:1.5 mL) was degassed for 5 min using a steady stream of nitrogen. t-Butylamine (0.25 mL, 2.38 mmol) was added, and the mixture was degassed for 5 min under nitrogen. The reaction mixture was reacted in the microwave at 160° C. for 40 min prior to being cooled to room temperature and concentrated under reduced pressure. The reaction mixture was suspended in EtOH (4.5 mL) to which hydrazine hydrate (0.15 mL, 3.00 mmol) was added prior to being reacted in the microwave at 165° C. for 30 min. Once cooled to room temperature the reaction mixture was concentrated under reduced pressure. Purification by column chromatography (100 EtOAc—10 % MeOH/EtOAc—20 % MeOH/EtOAc) followed by recrystallisation with MeOH/EtOAc and filtration afforded the desired compound as an off white solid (0.071 g, 0.17 mmol, 60.7 %). ¹H NMR (DMSO-d₆ ): δ 2.95 (t, J=14.4 Hz, 2 H), 3.55 (q, J=6.3 Hz, 2 H), 5.48 (br s, 2 H), 6.62 (t, J=11.2 Hz, 1 H), 6.72 (s, 1 H), 6.80 (d, J=5.6 Hz, 1 H), 7.30 (m, 3 H), 7.46 (d, J=8.0 Hz, 2 H), 7.53 (d, J=10.0 Hz, 1 H), 7.76 (d, J=8.4 Hz, 2 H), 8.03 (d, J=5.6 Hz, 1 H), 8.07 (s, 1 H), 11.51 (br s, 1 H). HRMS: For C₂₀H₂₁O₂N₆ S requires 409.1441, found 409.1435 . (M+H)⁺

### 5 -(2 -((3 -Chlorophenethyl)amino)pyridin-4 -yl)-1 H-indazol-3 -amine SU1215

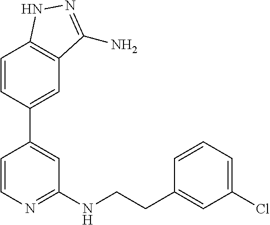 [SMILES: Nc1n[nH]c2ccc(-c3ccnc(NCCc4cccc(Cl)c4)c3)cc12]

A suspension of 4 -bromo-N-[2 -(3 -chlorophenyl)ethyl]pyridin-2 -amine (0.110 g, 0.35 mmol), 2 -fluoro-5 -(4,4,5,5 -tetramethyl-1,3,2 -dioxaborolan-2 -yl)benzonitrile (0.128 g, 0.49 mmol), [1,1 ′-bis(di-tert-butylphosphino)ferrocene]dichloropalladium(II) catalyst (0.045 g, 0.04 mmol) in IPA/H₂O (3:1.5 mL) was degassed for 5 min using a steady stream of nitrogen. t-Butylamine (0.20 mL, 2.10 mmol) was added, and the mixture was degassed for 5 min under nitrogen. The reaction mixture was reacted in the microwave at 160° C. for 40 min prior to being cooled to room temperature and concentrated under reduced pressure. The reaction mixture was suspended in EtOH (4.5 mL) to which hydrazine hydrate (0.15 mL, 3.0 mmol) was added prior to being reacted in the microwave at 165° C. for 30 min. Once cooled to room temperature the reaction mixture was concentrated under reduced pressure. Purification by column chromatography (100 % Hexane—50/50 Hexane/EtOAc—100 % EtOAc—10 % MeOH/EtOAc) followed by recrystallisation from MeOH (15 mL) and hexane (150 mL) and filtration afforded the desired compound as an off white solid (0.052 g, 0.14 mmol, 40.3 %). ¹H NMR (DMSO-d₆ ): δ 2.88 (t, J=17.0 Hz, 2 H), 3.53 (q, J=6.3 Hz, 2 H), 5.74 (br s, 2 H), 6.56 (t, J=11.6 Hz, 1 H), 6.71 (s, 1 H), 6.79 (m, 1 H), 7.72-7.37 (m, 5 H), 7.53 (d, J=8.4 Hz, 1 H), 8.02 (d, J=5.2 Hz, 1 H), 8.07 (1 H, s), 11.51 (1 H, br s). HRMS: For C₂₀H₁₉N₅Cl requires 364.1320, found 364.1323 . (M+H)⁺

### 5 -(2 -((2 -(Trifluoromethyl)phenethyl)amino)pyridin-4 -yl)-1 H-indazol-3 -amine SU1580

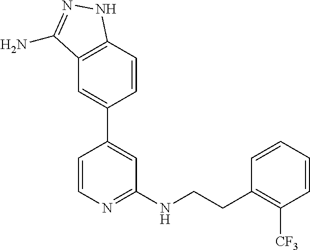 [SMILES: Nc1n[nH]c2ccc(-c3ccnc(NCCc4ccccc4C(F)(F)F)c3)cc12]

4 -Bromo-N-(2 -(trifluoromethyl)phenethyl)pyridin-2 -amine (48 mg, 0.14 mmol), 5 -(4,4,5,5 -Tetramethyl-1,3,2 -dioxaborolan-2 -yl)-1 H-indazol-3 -amine (43 mg, 0.17 mmol), [1,1 ′-bis(di-tert-butylphosphino)ferrocene]dichloropalladium(II) catalyst (9 mg, 0.014 mmol) and ethanol (0.4 mL) were placed in a 2-5 mL microwave vial which was sealed and purged with nitrogen for 5 mins. K₃ PO₄ (1 M, 0.2 mL) was added, and the solution stirred at 70° C. for 24 hours. Organics were extracted into EtOAc (50 mL), washed with water and dried over magnesium sulfate. This was filtered and organics adsorbed onto silica. Chromatographic purification (Biotage SP4, 50 g cartridge, solvent system: EtOAc/methanol, 0 % 2 CV; 0-5 % 3 CV; 5 % 4 CV) yielded title product as a brown/green solid (28 mg, 51 %). ¹ H (DMSO-d₆, 400 MHz) δ 3.06 (t, J=7.4 Hz, 2 H), 3.56 (q, J=6.9 Hz, 2 H), 5.46 (s, 2 H), 6.72 (m, 2 H), 6.81 (dd, J=5.3 & 1.5 Hz, 1 H), 7.32 (dd, J=8.8 & 0.5 Hz, 1 H), 7.44 (t, J=7.5 Hz, 1 H), 7.54 (m, 2 H), 7.64 (t, J=7.5 Hz, 1 H), 7.71 (d, J=7.8 Hz, 1 H), 8.05 (d, J=5.3 Hz, 1 H), 8.08 (d, J=1 Hz, 1 H), 11.51 (br s, 1 H); ¹³ C (DMSO-d₆, 100 MHz) δ 32.56, 42.76, 105.13, 110.32, 110.43, 115.06, 119.06, 120.25, 125.08 (d, J=288.2 Hz), 125.35, 126.17 (d, J=5.7 Hz), 127.12, 128.07 (d, J=36 Hz), 132.20, 132.93, 138.89 (d, J=1.5 Hz), 141.85, 148.60, 149.19, 150.29, 159.79 ; HRMS (ESI+ve): For C₂₁H₁₈ F3N₅ requires 398.1587 found 398.1587 . (M+H)⁺

### 5 -(2 -((3 -Phenylpropyl)amino)pyridine-4 -yl)-1 H-indazole-3 -amine SU1124

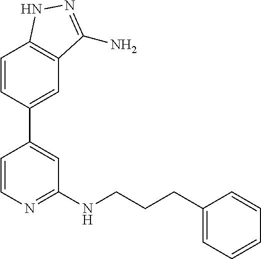 [SMILES: Nc1n[nH]c2ccc(-c3ccnc(NCCCc4ccccc4)c3)cc12]

A suspension of 4 -bromo-N-(3 -phenylpropyl)pyridine-2 -amine (0.101 g, 0.348 mmol), 3 -cyano-4 -fluorophenylboronic acid (0.137 g, 0.55 mmol), [1,1 ′-bis(di-tert-butylphosphino)ferrocene]dichloropalladium(II) catalyst (0.027 g, 0.041 mmol) in IPA/H₂O (3:1.5 mL) was degassed with nitrogen. t-Butylamine (0.20 mL, 1.90 mmol) was then added and degassed using nitrogen. The reaction was placed in a microwave and irradiated at 160° C. for 40 min. The reaction was diluted with EtOAc and concentrated under reduced pressure. EtOH (4 mL) was added followed by hydrazine hydrate (0.2 mL, 5.0 mmol) and the mixture irradiated at 165° C. for 30 min. The reaction mixture was then concentrated under reduced pressure and purified using column chromatography (100 % EtOAc). The resulting solid was triturated with diethyl ether, filtered and dried to give the title compound as an off-white solid (0.041 g, 0.12 mmol, 34 %). ¹H NMR (400 MHz, DMSO-d₆ ): δ 1.86 (qt, J=7.4 Hz, 2 H), 2.68 (t, J=7.6 Hz, 2 H), 3.28 (q, J=6.7 Hz, 2 H), 5.48 (br s, 2 H), 6.57 (t, J=5.4 Hz, 1 H), 6.70 (s, 1 H), 6.76 (dd, J=1.2, 5.2 Hz, 1 H), 7.18 (t, J=7.2, 14.0 Hz, 1 H), 7.23-7.31 (m, 5 H), 7.52 (dd, J=1.6, 8.8 Hz, 1 H), 7.99 (d, J=5.2 Hz, 1 H), 8.07 (s, 1 H), 11.51 (br s, 1 H). HRMS: For C₂₁H₂₂N₅ requires 344.1870 found 344.1866 . (M+H)⁺

### 5 -{2 -[(2 -Phenoxyethyl)amino]pyridine-4 -y1 }-1 H-indazol-3 -amine SU1203

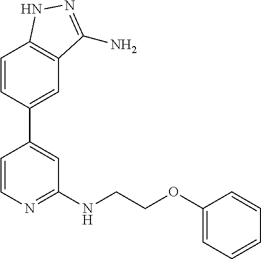 [SMILES: Nc1n[nH]c2ccc(-c3ccnc(NCCOc4ccccc4)c3)cc12]

A suspension of 4 -bromo-N-(2 -phenoxyethyl)pyridin-2 -amine (0.60 g, 0.21 mmol), 2 -fluoro-5 -(4,4,5,5 -tetramethyl-1,3,2 -dioxaborolan-2 -yl)benzonitrile (0.092 g, 0.37 mmol), [1,1 ′-bis(di-tert-butylphosphino)ferrocene]dichloropalladium(II) catalyst (0.030 g, 0.046 mmol) in IPA/H₂O (3:1.5 mL) was degassed for 5 min using a steady stream of nitrogen. t-Butylamine (0.15 mL, 1.43 mmol) was added, and the mixture was degassed for 5 min under nitrogen. The reaction mixture was reacted in the microwave at 160° C. for 40 min prior to being cooled to room temperature and concentrated under reduced pressure. The reaction mixture was suspended in EtOH (4.5 mL) to which hydrazine hydrate (0.15 mL, 3.0 mmol) was added prior to being reacted in the microwave at 165° C. for 30 min. Once cooled to room temperature the reaction mixture was concentrated under reduced pressure. Purification by column chromatography (100 % EtOAc) followed by recrystallisation from MeOH (15 mL) and hexane (150 mL) and filtration afforded the desired compound as a yellow solid (0.0066 g, 0.019 mmol, 9.3 %). ¹ H NMR (DMSO-d₆ ): δ 3.70 (q, J=5.7 Hz, 2 H), 4.13 (t, J=5.8 Hz, 2 H), 5.45 (br s, 2 H), 6.74 (t, J=5.4 Hz, 1 H), 6.82 (m, 2 H), 6.97 (m, 3 H), 7.30 (m, 3 H), 7.53 (dd, J=8.8, 1.6 Hz, 1 H,), 8.03 (d, J=5.6 Hz, 1 H), 8.08 (s, 1 H), 11.49 (br s, 1 H). HRMS: For C₂₀H₂₀0N₅ requires 346.1665, found 346.1662 . (M+H)⁺

### Amide Substituents at Position 2 of the Aminopyridine Ring

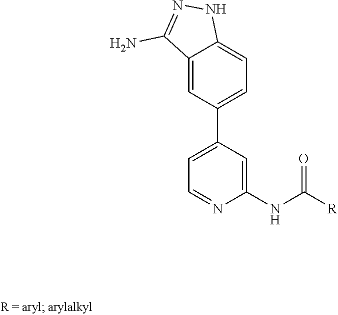

### N-(4 -(3 -Amino-1 H-indazol-5 -yl)pyridin-2 -yl)-2 -phenylacetamide SU1244

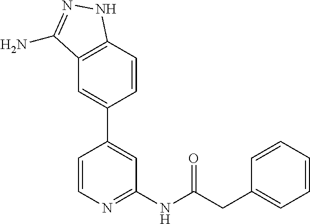 [SMILES: Nc1n[nH]c2ccc(-c3ccnc(NC(=O)Cc4ccccc4)c3)cc12]

5 -(4,4,5,5 -Tetramethyl-1,3,2 -dioxaborolan-2 -yl)-1 H-indazol-3 -amine (100 mg, 0.38 mmol), N-(4 -chloropyridin-2 -yl)-2 -phenylacetamide (78 mg, 0.32 mmol) in ethanol (O₂ -free, 0.8 mL) and

′-bis(di-tert-butylphosphino)ferrocene]dichloropalladium(II) catalyst (21 mg, 0.032 mmol) were placed in a 2-5 mL microwave vial which was sealed and purged with nitrogen for 5 mins.

The suspension stirred at 50° C. under nitrogen for a further 10 mins. K₃ PO₄ (1 M, 0.4 mL) was added and the solution stirred at 70° C. for 24 hours. Reaction was quenched with water (5 mL) to yield a brown solid which was extracted into EtOAc and adsorbed onto silica under reduced pressure. Chromatographic purification (Biotage SP4, 50 g cartridge, solvent system: EtOAc/methanol, 0 % 2 CV; 0-5 % 3 CV; 5 % 4 CV) yielded title product as a grey/brown solid (65 mg, 57 %). ¹ H (DMSO-d₆, 400 MHz) δ 3.77 (s, 2 H), 5.53 (s, 2 H), 7.26 (m, 1 H), 7.34 (m, 3 H), 7.39 (m, 3 H), 7.59 (dd, J=8.8 & 1.8 Hz, 1 H), 8.15 (d, J=1 Hz, 1 H), 8.35 (d, J=5.3 Hz, 1 H), 8.44 (s, 1 H), 10.74 (s, 1 H), 11.58 (br s, 1 H); ¹³ C (DMSO-d₆, 100 MHz) δ 43.51, 110.58, 115.14, 117.34, 119.66, 125.50, 127.05, 127.38, 128.76 (2 C), 129.74 (2 C), 136.29, 142.00, 148.81, 150.45, 150.59, 15327, 170.61 ; HRMS (ESI+ve): For C₂₀H₁₇N₅O requires 344.1506 found 344.1503 . (M+H)⁺

### N-(4 -(3 -Amino-1 H-indazol-5 -yl)pyridin-2 -yl)-2 -(3 -fluorophenyl)acetamide SU1287

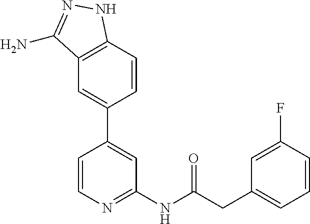 [SMILES: Nc1n[nH]c2ccc(-c3ccnc(NC(=O)Cc4cccc(F)c4)c3)cc12]

To a suspension of N-(4 -bromopyridin-2 -yl)-2 -(3 -fluorophenyl)acetamide (0.179 g, 0.58 mmol), 5 -(4,4,5,5 -tetramethyl-1,3,2 -dioxaborolan-2 -yl)-1 H-indazol-3 -amine (0.192 g, 0.74 mmol) and Tetrakis(triphenylphosphine)palladium(0 ) catalyst (0.082 g, 0.071 mmol) in EtOH (3.0 mL, degassed under nitrogen) was added K₃ PO₄ (1 M, 1.2 mL) and the reaction mixture was heated to 80° C. for 24 h. The reaction mixture was cooled to room temperature and the reaction mixture was diluted with EtOAc, washed with water, aqueous saturated NaHCO₃ and brine. Organics were concentrated under reduced pressure and purified by column chromatography (100 % Hexane—2/1 Hexane/EtOAc—100 % EtOAc) and triturated Et₂O to afford the title compound as an off-white solid (0.0685 g, 0.19 mmol, 32.6 %). ¹H NMR (DMSO-d₆ ): δ 3.80 (s, 2 H), 5.52 (br s, 2 H), 7.09 (m, 1 H), 7.21 (m, 2 H), 7.37 (m, 3 H), 7.58 (dd, J=1.6, 8.8 Hz, 1 H), 8.15 (s, 1 H), 8.34 (d, J=5.2 Hz, 1 H), 8.42 (s, 1 H), 10.76 (br s, 1 H), 11.55 (br s, 1 H). HRMS: For C₂₀H₁₇ON₅ F requires 362.1412 found 362.1417 . (M+H)⁺

### N-(4 -(3 -Amino-1 H-indazol-5 -yl)pyridin-2 -yl)-2 -(3 -tolyl)acetamide SU1577

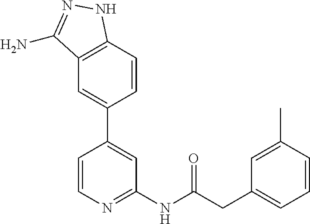 [SMILES: Cc1cccc(CC(=O)Nc2cc(-c3ccc4[nH]nc(N)c4c3)ccn2)c1]

5 -(4,4,5,5 -Tetramethyl-1,3,2 -dioxaborolan-2 -yl)-1 H-indazol-3 -amine (85 mg, 0.325 mmol), N-(4 -chloropyridin-2 -yl)-2 -(3 -tolyl)acetamide (65 mg, 0.25 mmol) and [1,1 ′-bis(di-tert-butylphosphino)ferrocene]dichloropalladium(II) catalyst (9 mg, 0.0125 mmol) were placed in a 2-5 mL microwave vial which was sealed and purged with nitrogen for 5 mins. Ethanol (O₂ -free, 1 mL) was added, and the suspension stirred at 50° C. under nitrogen for a further 5 mins. K₃ PO₄ (1 M, 0.5 mL) was added, and the solution stirred at 100° C. for 24 hours. Reaction was quenched with water (8 mL) to yield a brown solid which was filtered and further washed with water (15×10 mL) and hexane (8×10 mL). Solid was resuspended in 50 % EtOAc/methanol (20 mL), filtered and adsorbed onto silica. Chromatographic purification (Biotage SP4, 50 g cartridge, solvent system: 1 % NEt₃ in EtOAc/methanol, gradient: 0 % 3 CV; 0-5 % 2 CV; 5 % 4 CV) yielded title product as a pale brown solid (35.8 mg, 40 %). ¹ H (DMSO-d₆, 400 MHz) δ 2.30 (s, 3 H), 3.71 (s, 2 H), 5.55 (s, 2 H), 7.06 (d, J=7.5 Hz, 1 H), 7.17 (m, 2 H), 7.22 (t, J=7.5 Hz, 1 H), 7.33 (d, J=8.8 Hz, 1 H), 7.59 (dd, J=5.3 & 1.8 Hz, 1 H), 7.58 (d, J=8.8 Hz, 1 H), 8.34 (d, J=5.3 Hz, 1 H), 8.43 (s, 1 H), 10.74 (s, 1 H), 11.58 (s, 1 H); ¹³ C (DMSO-d₆, 100 MHz) δ 20.95, 42.95, 110.05 (2 C), 114,72, 116.81, 119.14, 124.98, 126.30, 127.18, 128.17, 128.14, 128.17, 129.86, 135.66, 137.29, 148.30, 150.07, 152.78, 170.15 ; HRMS: For C₂₁H₁₉N₅O requires 358.1662 found 358.1656 . (M+H)⁺

### N-(4 -(3 -Amino-1 H-indazol-5 -yl)pyridin-2 -yl)-2 -(3 -trifluoromethyl)acetamide SU1565

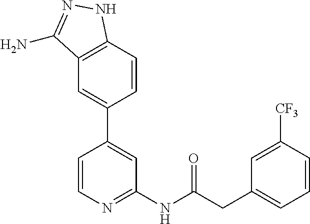 [SMILES: Nc1n[nH]c2ccc(-c3ccnc(NC(=O)Cc4cccc(C(F)(F)F)c4)c3)cc12]

5 -(4,4,5,5 -Tetramethyl-1,3,2 -dioxaborolan-2 -yl)-1 H-indazol-3 -amine (118 mg, 0.45 mmol), N-(4 -chloropyridin-2 -yl)-2 -(3 -(trifluoromethyl)phenyl)acetamide (126 mg, 0.4 mmol) and [1,1 ′-bis(di-tert-butylphosphino)ferrocene]dichloropalladium(II) catalyst (26 mg, 0.04 mmol) were placed in a 2-5 mL microwave vial which was sealed and purged with nitrogen for 5 mins. Ethanol (O₂ -free, 1.12 mL) was added and the suspension stirred at 40° C. under nitrogen for a further 10 mins. K₃ PO₄ (1 M, 0.56 mL) was added and the solution stirred at 70° C. for 24 hours. Reaction was quenched with water (5 mL) to yield a yellowish solid. Organics were extracted into EtOAc (50 mL) and adsorbed onto silica under reduced pressure. . Chromatographic purification (Biotage SP4, 50 g cartridge, solvent system: EtOAc/methanol, gradient: 0 % 2 CV; 0-2 % 2 CV; 2 % 4 CV) yielded title product as a pale yellow solid (71 mg, 43 %); ¹ H (DMSO-d₆, 400 MHz) b 3.91 (s, 2 H), 5.53 (s, 2 H), 7.33, (d, J=8.5 Hz, 1 H), 7.41 (dd, J=5.3 & 1.8 Hz, 1 H), 7.60 (m, 3 H), 7.71 (s, 1 H), 7.72 (s, 1 H), 8.16 (d, J=1 Hz, 1 H), 8.35 (d, J=5.3 Hz, 1 H), 8.43 (s, 1 H), 10.84 (s, 1 H), 11.62 (br s, 1 H); ¹³ C (DMSO-d₆, 100 MHz) δ 43.12, 110.62, 115.14, 117.43, 119.66, 124.86 (d, J=271.6 Hz), 125.47, 125.58 (q, 3.7 Hz), 127.33, 127.85 (d, J=31.2 Hz), 130.66 (2 C), 130.80, 141.09, 142.00, 148.84, 150.46, 150.63, 153.16, 169.89 ; HRMS: C₂₁H₁₇ F3N₅O requires 412.1380 found 412.1372 . (M+H)⁺

### N-(4 -(3 -Amino-1 H-indazol-5 -yl)pyridin-2 -yl)-2 -(3 -chlorophenyl)acetamide SU1560

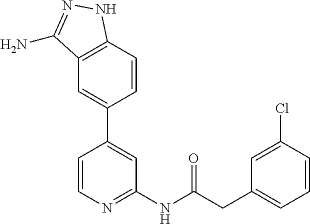 [SMILES: Nc1n[nH]c2ccc(-c3ccnc(NC(=O)Cc4cccc(Cl)c4)c3)cc12]

5 -(4,4,5,5 -Tetramethyl-1,3,2 -dioxaborolan-2 -yl)-1 H-indazol-3 -amine (165 mg, 0.6 mmol), N-(4 -bromopyridin-2 -yl)-2 -(3 -chlorophenyl)acetamide (162.4 mg, 0.5 mmol) and [1,1 ′-bis(di-tert-butylphosphino)ferrocene]dichloropalladium(II) catalyst (16.2 mg, 0.025 mmol) were placed in a 2-5 mL microwave vial which was sealed and purged with nitrogen for 5 mins. Ethanol (O₂ -free, 1.3 mL) was added, and the suspension stirred at 40° C. under nitrogen for a further 10 mins. K₃ PO₄ (1 M, 0.65 mL) was added, and the solution stirred at 70° C. for 24 hours. Reaction was quenched with water (5 mL) to yield a yellowish solid. Organics were extracted into EtOAc (50 mL) and adsorbed onto silica under reduced pressure. Chromatographic purification (Biotage SP4, 50 g cartridge, solvent system: EtOAc/methanol, gradient: 0 % 2 CV; 0-2 % 2 CV; 2 % 4 CV) yielded title product as a brown/green powder (95 mg, 50 %) (LC-MS purity=97.2 %); ¹ H (DMSO-d₆, 500 MHz) δ 3.79 (s, 2 H), 5.52 (s, 2 H), 7.32, (m, 3 H), 7.36 (m, 1 H) 7.39 (dd, J=5.3 & 1.9 Hz, 1 H), 7.45 (t, J=1.7 Hz, 1 H), 7.58 (dd, J=8.8 & 1.6 Hz, 1 H), 8.14 (d, J=0.9 Hz, 1 H), 8.34 (d, J=5.3 Hz, 1 H), 8.41 (s, 1 H), 10.77 (s, 1 H), 11.56 (s, 1 H); 13 C (DMSO-d₆, 125 MHz) δ 42.90, 110.58 (2 C), 115.14, 117.41, 119.66, 125.49, 127.07, 127.33, 128.54, 129.69, 130.60, 133.29, 138.67, 142.00, 148.84, 150.46, 150.62, 153.18, 170.04 . HRMS: C₂₀H₁₇ ClN₅O requires 378.1116 found 378.1114 (M+H)⁺.

### N-(4 -(3 -Amino-1 H-indazol-5 -yl)pyridin-2 -yl)-2 -(3 -aminophenyl)acetamide SU1275

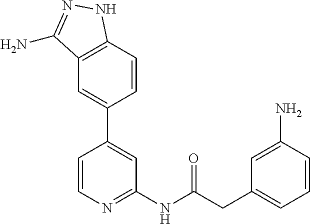 [SMILES: Nc1cccc(CC(=O)Nc2cc(-c3ccc4[nH]nc(N)c4c3)ccn2)c1]

5 -(4,4,5,5 -Tetramethyl-1,3,2 -dioxaborolan-2 -yl)-1 H-indazol-3 -amine (118 mg, 0.45 mmol), N-(4 -chloropyridin-2 -yl)-2 -(3 -Boc-aminophenyl)acetamide (145.5 mg, 0.4 mmol) and [1,1 ′-bis(di-tert-butylphosphino)ferrocene]dichloropalladium(II) catalyst (26 mg, 0.04 mmol) were placed in a 2 -5 mL microwave vial which was sealed and purged with nitrogen for 5 mins. Ethanol (O₂ -free, 1.12 mL) was added, and the suspension stirred at 50° C. under nitrogen for a further 10 mins. K₃ PO₄ (1 M, 0.56 mL) was added, and the solution stirred at 70° C. for 24 hours. Reaction was quenched with water (50 mL) to yield a yellowish solid. This was sonicated for 30 mins and resultant solid was filtered and resuspended in 80 % EtOAc/methanol (18 mL) and adsorbed onto silica. Product was obtained by chromatographic purification (Biotage SP4, 50 g cartridge, solvent system: EtOAc/methanol, gradient: 0 % 2 CV; 0-2 % 2 CV; 2 % 6 CV). Product was suspended in dichloromethane (3 mL) and trifluoracetic acid (1.5 mL) and stirred at room temperature for 8 hours. Solution was diluted with EtOAc (40 mL), washed with Na₂ CO₃ (sat., 20 mL) and concentrated under reduced pressure to yield title product (55 mg, 39 % over 2 steps). ¹ H (DMSO-d₆, 400 MHz) δ 3.58 (s, 2 H), 5.02 (s, 2 H), 5.52 (s, 2 H), 6.44 (ddd, J=8, 2.2 & 0.9 Hz, 1 H), 6.52 (d, J=7.5 Hz), 6.58 (m, 1 H), 6.96 (t, J=7.8 Hz, 1 H), 7.34 (d, J=8.5 Hz, 1 H), 7.38 (dd, J=5.3 & 1.8 Hz, 1 H), 7.59 (dd, J=8.8 & 1.8 Hz, 1 H), 8.15 (d, J=1 Hz, 1 H), 8.34 (d, J=5.3 Hz, 1 H), 8.44 (s, 1 H), 10.60 (s, 1 H), 11.56 (s, 1 H); 3 C (DMSO-d₆, 100 MHz) δ 43.83, 110.55, 110.58, 112.78, 115.09, 117.24, 117.28, 119.66, 125.50, 127.43, 129.22, 136.74, 142.00, 148.78, 149.09, 150.45, 150.57, 153.33, 170.83 ; HRMS: For C₂₀H₁₉N₆O requires 359.1615 found 359.1609 . (M+H)⁺

### N-(4 -(3 -Amino-1 H-indazol-5 -yl)pyridin-2 -yl)-2 -(3 -nitrophenyl)acetamide SU1273

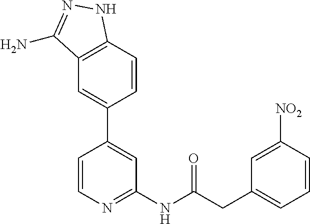 [SMILES: Nc1n[nH]c2ccc(-c3ccnc(NC(=O)Cc4cccc([N+](=O)[O-])c4)c3)cc12]

To a suspension of N-(4 -bromopyridin-2 -yl)-2 -(3 -nitrophenyl)acetamide (0.196 g, 0.59 mmol), 5 -(4,4,5,5 -tetramethyl-1,3,2 -dioxaborolan-2 -yl)-1 H-indazol-3 -amine (0.161 g, 0.62 mmol) and

′-bis(di-tert-butylphosphino)ferrocene]dichloropalladium(II) catalyst (0.042 g, 0.06 mmol) in EtOH (3.0 mL, degassed under nitrogen) was added K₃ PO₄ (1 M, 1.2 mL) and the reaction mixture was heated to 80° C. for 24 h. The reaction mixture was cooled to room temperature and the reaction mixture was diluted with EtOAc, washed with water, aqueous saturated NaHCO₃ and brine. Organics were concentrated under reduced pressure and purified by column chromatography (100 % Hexane—2/1 Hexane/EtOAc—100 % EtOAc—10 % MeOH/EtOAc) and triturated with MeOH and hexane to afford the title compound as an off-white solid (0.043 g, 0.11 mmol, 19.1 %). ¹H NMR (DMSO-dc): 6 3.96 (s, 2 H), 5.51 (s, 2 H), 7.32 (d, J=8.8 Hz, 1 H), 7.41 (dd, J=1.6, 5.6 Hz, 1 H), 7.58 (dd, J=1.6, 8.8 Hz, 1 H), 7.65 (t, J=8.0 Hz, 1 H), 7.83 (d, J=8.0 Hz, 1 H), 8.14 (m, 2 H), 8.28 (m, 1 H), 8.35 (d, J=5.2 Hz, 1 H), 8.41 (s, 1 H), 10.86 (s, 1 H), 11.55 (s, 1 H). HRMS: For C₂₀H₁₇03N₆ requires 389.1357 found 389.1353 . (M+H)⁺

### N-(4 -(3 -Amino-1 H-indazol-5 -yl)pyridin-2 -yl)-2 -(3 -(methylsulfonyl)phenyl)acetamide SU1574

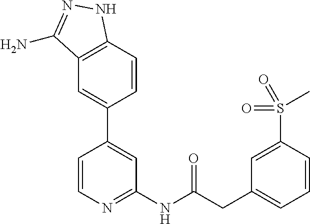 [SMILES: CS(=O)(=O)c1cccc(CC(=O)Nc2cc(-c3ccc4[nH]nc(N)c4c3)ccn2)c1]

5 -(4,4,5,5 -Tetramethyl-1,3,2 -dioxaborolan-2 -yl)-1 H-indazol-3 -amine (118 mg, 0.45 mmol), N-(4 -chloropyridin-2 -yl)-2 -(3 -(methylsulfonyl)phenyl)acetamide (139 mg, 0.4 mmol) and [1,1 ′-bis(di-tert-butylphosphino)ferrocene]dichloropalladium(II) catalyst (23 mg, 0.035 mmol) were placed in a 2-5 mL microwave vial which was sealed and purged with nitrogen for 5 mins. Ethanol (O₂ -free, 1.12 mL) was added, and the suspension stirred at 50° C. under nitrogen for a further 10 mins. K₃ PO₄ (1 M, 0.56 mL) was added, and the solution stirred at 70° C. for 24 hours. Reaction was quenched with water (5 mL) to yield a yellowish solid. Supernatant was removed and solid resuspended in 80 % EtOAc/methanol (6 mL) and adsorbed onto silica under reduced pressure.

Chromatographic purification (Biotage SP4, 50 g cartridge, solvent system: EtOAc/methanol, gradient: 0 % 2 CV; 0-5 % 3 CV; 5 % 4 CV) yielded title product as a greenish solid (61 mg, 34 %). ¹ H (DMSO-d₆, 400 MHz) δ 3.23 (s, 3 H), 3.93 (s, 2 H), 5.53 (s, 2 H), 7.34 (d, J=8.5 Hz, 1 H), 7.41 (dd, J=5.3 & 1.8 Hz, 1 H), 7.59 (dd, J=8.8 & 1.8 Hz, 1 H), 7.64 (t, J=7.8 Hz, 1 H), 7.73 (m, 1 H), 7.85 (ddd, J=7.6, 1.6 & 1.3 Hz, 1 H), 7.97 (m, 1 H), 8.16 (d, J=1 Hz, 1 H), 8.36 (dd, J=5.3 & 0.5 Hz, 1 H), 8.43 (s, 1 H), 10.86 (s, 1 H), 11.56 (s, 1 H); ¹³ C (DMSO-d₆, 100 MHz) δ 42.92, 44.00, 110.56, 110.59, 115.11, 117.43, 119.68, 125.50, 125.80, 127.28, 128.04, 129.90, 135.10, 137.73, 141.30, 141.96, 148.87, 150.47, 150.62, 153.15, 169.92 ; HRMS: For C₂₁H₂₀N₅O₃ S requires 422.1281 found 422.1279 . (M+H)⁺

### N-(4 -(3 -Amino-1 H-indazol-5 -yl)pyridin-2 -yl)-2 -(3 -methoxyphenyl)acetamide SU1572

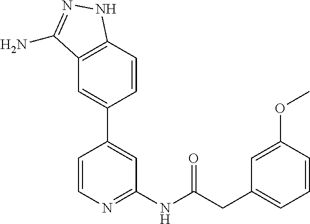 [SMILES: COc1cccc(CC(=O)Nc2cc(-c3ccc4[nH]nc(N)c4c3)ccn2)c1]

5 -(4,4,5,5 -Tetramethyl-1,3,2 -dioxaborolan-2 -yl)-1 H-indazol-3 -amine (110 mg, 0.42 mmol), N-(4 -chloropyridin-2 -yl)-2 -(3 -methoxyphenyl)acetamide (97 mg, 0.35 mmol) and [1,1 ′-bis(di-tert-butylphosphino)ferrocene]dichloropalladium(II) catalyst (23 mg, 0.035 mmol) were placed in a 2-5 mL microwave vial which was sealed and purged with nitrogen for 5 mins. Ethanol (O₂ -free, 1 mL) was added, and the suspension stirred at 50° C. under nitrogen for a further 5 mins. K₃ PO₄ (1 M, 0.5 mL) was added, and the solution stirred at 70° C. for 24 hours. Reaction was quenched with water (5 mL) to yield a yellowish solid. Supernatant was removed and solid was resuspended in 80 % EtOAc/methanol (6 mL) and adsorbed onto silica. Chromatographic purification (Biotage SP4, 50 g cartridge, solvent system: EtOAc/methanol, gradient: 0 % 2 CV; 0-5 % 3 CV; 5 % 6 CV) yielded title product as a pale brown solid (82 mg, 63 %). ¹ H (DMSO-d₆, 400 MHz) δ 3.72 (s, 2 H), 3.75 (s, 3 H), 5.55 (s, 2 H), 6.83 (d, J=7.9 Hz, 1 H), 6.94 (m, 2 H), 7.24 (t, J=7.7 Hz, 1 H), 7.33 (d, J=8.8 Hz, 1 H), 7.39 (d, J=5.3 Hz, 1 H), 7.59 (d, J=8.8 Hz, 1 H), 8.15 (s, 1 H), 8.34 (d, J=5.3 Hz, 1 H), 8.43 (s, 1 H), 10.74 (s, 1 H), 11.58 (br s, 1 H); ¹ C (DMSO-d₆, 100 MHz) δ 43.56, 55.44, 110.52, 110.57, 112.43, 115.11, 115.55, 117.34, 119.67, 121.96, 125.50, 127.34, 129.79, 137.70, 141.95, 148.83, 150.46, 150.59, 153.25, 159.66, 170.48 ; HRMS: For C₂₁H₂₀N₅O₂ requires 374.1612 found 374.1606 . (M+H)⁺

### N-(4 -(3 -Amino-1 H-indazol-5 -yl)pyridin-2 -yl)-2 -(3 -benzyloxy)phenyl)acetaminde SU1323

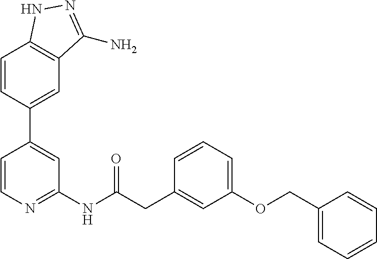 [SMILES: Nc1n[nH]c2ccc(-c3ccnc(NC(=O)Cc4cccc(OCc5ccccc5)c4)c3)cc12]

To a suspension of 2 -(3 -(benzyloxy)phenyl)-N-(4 -bromopyridin-2 -yl)acetamide (0.257 g, 0.68 mmol), 5 -(4,4,5,5 -tetramethyl-1,3,2 -dioxaborolan-2 -yl)-1 H-indazol-3 -amine (0.202 g, 0.78 mmol) and [1,1 ′-bis(di-tert-butylphosphino)ferrocene]dichloropalladium(II) catalyst (0.044 g, 0.07 mmol) in EtOH (3.0 mL, degassed under nitrogen) was added K₃ PO₄ (1 M, 1.2 mL) and the reaction mixture was heated to 80° C. for 20 h. The reaction mixture was cooled to room temperature and diluted with EtOAc, washed with water, aqueous saturated NaHCO₃ and brine.

The organics were concentrated under reduced pressure and the resulting residue and purified by column chromatography (100 % Hexane—1/1 Hexane/EtOAc—100 % EtOAc—10 % MeOH/EtOAc) and triturated with Et₂O to afford the title compound as a white solid (0.137 g, 0.30 mmol, 60 %). ⁴ H NMR (DMSO-d₆ ): δ 3.72 (s, 2 H), 5.09 (br s, 2 H), 5.52 (br s, 2 H), 6.90 (dd, J=1.6, 6.4 Hz, 1 H), 6.95 (d, J=6.0 Hz, 1 H), 7.05 (br s, 1 H), 7.24 (t, J=4.1 Hz, 1 H), 7.37 (m, 5 H), 7.45 (d, J=6.0 Hz, 2 H), 7.59 (d, J=7.2 Hz, 1 H), 8.14 (s, 1 H), 8.34 (d, J=4.4 Hz, 1 H), 8.42 (s, 1 H), 10.71 (br s, 1 H), 11.56 (br s, 1 H). HRMS: For C₂₇H₂₄O₂N₅ requires 450.1925 found 450.1920 . (M+H)⁺

### tert-Butyl-(3 -(2 -((4 -(3 -amino-1 H-indazol-5 -yl)pyridin-2 -yl)amino)-2 -oxoethyl)phenyl)carbamate SU1568

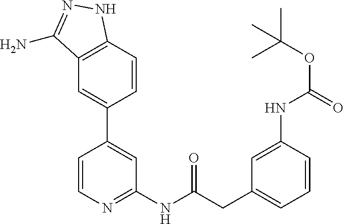 [SMILES: CC(C)(C)OC(=O)Nc1cccc(CC(=O)Nc2cc(-c3ccc4[nH]nc(N)c4c3)ccn2)c1]

5 -(4,4,5,5 -Tetramethyl-1,3,2 -dioxaborolan-2 -yl)-1 H-indazol-3 -amine (120 mg, 0.46 mmol), N-(4 -chloropyridin-2 -yl)-2 -(3 -Boc-aminophenyl)acetamide (120 mg, 0.33 mmol) and [1,1 ′-bis(di-tert-butylphosphino)ferrocene]dichloropalladium(II) catalyst (21.6 mg, 0.033 mmol) were placed in a 2-5 mL microwave vial which was sealed and purged with nitrogen for 5 mins. Ethanol (O₂ -free, 1 mL) was added, and the suspension stirred at 50° C. under nitrogen for a further 10 mins. K₃ PO₄ (1 M, 0.5 mL) was added, and the solution stirred at 70° C. for 24 hours. Reaction was quenched with water (50 mL) to yield a yellowish solid. This was sonicated for 30 mins and resultant solid was filtered and resuspended in 80 % EtOAc/methanol (18 mL) and adsorbed onto silica. Chromatographic purification (Biotage SP4, 50 g cartridge, solvent system: EtOAc/methanol, gradient: 0 % 2 CV; 0-5 % 3 CV; 5 % 6 CV) yielded title product as a brown solid (30 mg, 20 %) (61 mg, 40 %) (NMR purity=90 %); ¹ H (DMSO-d₆, 400 MHz) δ 1.47 (s, 9 H), 3.71 (s, 2 H), 5.52 (s, 2 H), 6.98 (d, J=7.5 Hz, 1 H), 7.20 (t, J=7.8 Hz, 1 H), 7.27 (d, J=8 Hz, 1 H), 7.34 (d, J=8.8 Hz, 1 H), 7.39 (dd, J=5.3 & 1.8 Hz, 1 H), 7.55 (s, 1 H), 7.59 (dd, J=8.8, 1.8 Hz, 1 H), 8.15 (d, J=0.8 Hz, 1 H), 8.34 (d, J=5.5 Hz, 1 H), 8.43 (s, 1 H), 9.31 (s, 1 H), 10.72 (s, 1 H), 11.56 (s, 1 H); ¹³ C (DMSO-d₆, 100 MHz) δ 28.59, 79.46, 110.59, 110.62, 115.10, 117.13, 117.36, 119.48, 119.64, 123.62, 125.54, 127.43, 128.96, 136.71, 139.95, 142.00, 148.83, 150.44, 150.57, 153.24, 153.28, 170.60.

### N-(4 -(3 -(Amino-1 H-indazol-5 -yl)pyridin-2 -yl)-2 -(4 -fluorophenyl)acetamide SU1264

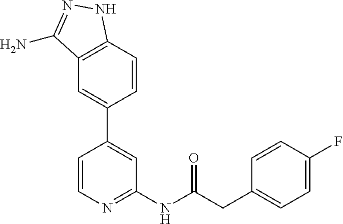 [SMILES: Nc1n[nH]c2ccc(-c3ccnc(NC(=O)Cc4ccc(F)cc4)c3)cc12]

To a suspension of N-(4 -bromopyridin-2 -yl)-2 -(4 -fluorophenyl)acetamide (0.176 g, 0.57 mmol), ), 5 -(4,4,5,5 -Tetramethyl-1,3,2 -dioxaborolan-2 -yl)-1 H-indazol-3 -amine (0.146 g, 0.67 mmol) and Tetrakis(triphenylphosphine)palladium(0 ) catalyst (0.077 g, 0.067 mmol) in EtOH (3.0 mL, degassed under nitrogen) was added K₃ PO₄ (1 M, 1.2 mL) and the reaction mixture was heated to 80° C. for 24 h. The reaction mixture was cooled to room temperature and the reaction mixture was filtered and the resulting solid was washed with water (10 mL) and Et₂O (8 mL) and then dried. Purification by column chromatography (100 % Hexane—2/1 Hexane/EtOAc—100 % EtOAc) and trituration with MeOH and hexane afforded the title compound as an off-white solid (0.058 g, 0.16 mmol, 27.9 %). ¹H NMR (DMSO-de): 6 3.76 (s, 2 H), 5.51 (br s, 2 H), 7.15 (m, 2 H), 7.33 (d, J=8.4 Hz, 1 H), 7.40 (m, 3 H), 7.57 (dd, J=1.6, 8.8 Hz, 1 H), 8.14 (s, 1 H), 8.34 (d, J=5.2 Hz), 8.41 (s, 1 H), 10.73 (br s, 1 H), 11.55 (br s, 1 H). HRMS: For C₂₀H₁₇ON₅ F requires 362.1412 found 362.1414 . (M+H)⁺

### N-(4 -(3 -Amino-1 H-indazol-5 -yl)pyridin-2 -yl)-2 -(4 -tolyl)acetamide SU1578

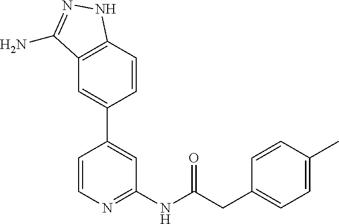 [SMILES: Cc1ccc(CC(=O)Nc2cc(-c3ccc4[nH]nc(N)c4c3)ccn2)cc1]

5 -(4,4,5,5 -Tetramethyl-1,3,2 -dioxaborolan-2 -yl)-1 H-indazol-3 -amine (110 mg, 0.42 mmol), N-(4 -chloropyridin-2 -yl)-2 -(4 -tolyl)acetamide (91 mg, 0.35 mmol) and [1,1 ′-bis(di-tert-butylphosphino)ferrocene]dichloropalladium(II) catalyst (23 mg, 0.035 mmol) were placed in a 2-5 mL microwave vial which was sealed and purged with nitrogen for 5 mins. Ethanol (O₂ -free, 1 mL) was added, and the suspension stirred at 50° C. under nitrogen for a further 5 mins. K₃ PO₄ (1 M, 0.5 mL) was added, and the solution stirred at 70° C. for 24 hours. Reaction was quenched with water (5 mL) to yield a yellowish solid. Supernatent was removed and solid was resuspended in 80 % EtOAc/methanol (6 mL) and adsorbed onto silica. Chromatographic purification (Biotage SP4, 50 g cartridge, solvent system: EtOAc/methanol, gradient: 0 % 2 CV: 2 % 8 CV) yielded title product as a pale brown solid (52 mg, 42 %). ¹ H (DMSO-d₆, 400 MHz) δ 2.28 (s, 3 H), 3.71 (s, 2 H), 5.53 (s, 2 H), 7.14 (d, J=7.8 Hz, 2 H), 7.28 (d, J=8 Hz, 2 H), 7.34 (d, J=8.5 Hz, 1 H), 7.39 (dd, J=5.3 & 1.8 Hz, 1 H), 7.59 (dd, J=8.8 & 1.8 Hz, 1 H), 8.15 (s, 1 H), 8.34 (d, J=5.5 Hz, 1 H), 8.42 (2, 1 H), 10.68 (s, 1 H), 11.56 (s, 1 H); ¹³ C (DMSO-d₆, 100 MHz) δ 21.12, 43.13, 110.56, 115.14, 117.31, 119.64, 125.49, 127.39, 129.32 (2 C), 129.59 (2 C), 133.22, 136.08, 141.99, 148.79, 150.45, 150.58, 153.29, 170.78 ; HRMS: For C₂₁H₁₉N₅₀ requires 358.1662 found 358.1660 . (M+H)⁺

### N-(4 -(3 -Amino-1 H-indazol-5 -yl)pyridin-2 -yl)-2 -(4 -(trifluoromethyl)phenyl)acetamide SU1566

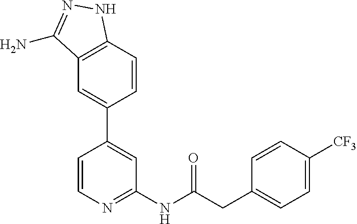 [SMILES: Nc1n[nH]c2ccc(-c3ccnc(NC(=O)Cc4ccc(C(F)(F)F)cc4)c3)cc12]

5 -(4,4,5,5 -Tetramethyl-1,3,2 -dioxaborolan2 -yl)-1 H-indazol-3 -amine (118 mg, 0.45 mmol), N-(4 -chloropyridin-2 -yl)-2 -(4 -(trifluoromethyl)phenyl)acetamide (126 mg, 0.4 mmol) and [1,1 ′-bis(di-tert-butylphosphino)ferrocene]dichloropalladium(II) catalyst (26 mg, 0.04 mmol) were placed in a 2-5 mL microwave vial which was sealed and purged with nitrogen for 5 mins. Ethanol (O₂ -free, 1.12 mL) was added, and the suspension stirred at 40° C. under nitrogen for a further 10 mins. K₃ PO₄ (1 M, 0.56 mL) was added, and the solution stirred at 70° C. for 24 hours. Reaction was quenched with water (5 mL) to yield a yellowish solid. Organics were extracted into EtOAc (50 mL) and adsorbed onto silica under reduced pressure. . Chromatographic purification (Biotage SP4, 50 g cartridge, solvent system: EtOAc/methanol, gradient: 0 % 2 CV; 0-2 % 2 CV; 2 % 4 CV) yielded title product as a pale yellow solid (78 mg, 47 %)¹ H (DMSO-de, 400 MHz) b 3.92 (s, 2 H), 5.53 (s, 2 H), 7.34, (d, J=8.8 Hz, 1 H), 7.41 (dd, J=5.3 & 1.8 Hz, 1 H), 7.59 (m, 2 H), 7.64 (d, J=7.6 Hz, 1 H), 7.68 (d, J=7.6 Hz, 1 H), 7.76 (s, 1 H), 8.16 (d, J=1 Hz, 1 H), 8.36 (d, J=5.3 Hz, 1 H), 8.43 (s, 1 H), 10.83 (s, 1 H), 11.63 (br s, 1 H); 13 C (DMSO-d₆, 100 MHz) δ 42.92, 110.59, 115.14, 117.43, 119.66, 123.87 (q, J=3.7 Hz), 124.86 (d, J=272.7 Hz), 125.50, 126.44 (q, J=3.7 Hz), 127.32, 129.45 (d, J=31.2 Hz), 129.78 (2 C), 134.03, 137.60, 142.01, 148.84, 150.46, 150.64, 153.16, 170.01 . HRMS: C₂₁H₁₇ F3N₅O requires 412.1380 found 412.1374 . (M+H)⁺

### N-(4 -(3 -Amino-1 H-indazol-5 -yl)pyridin-2 -yl)-2 -(4 -chlorophenyl)acetamide SU1561

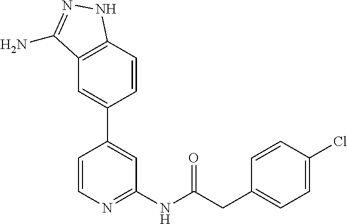 [SMILES: Nc1n[nH]c2ccc(-c3ccnc(NC(=O)Cc4ccc(Cl)cc4)c3)cc12]

5 -(4,4,5,5 -Tetramethyl-1,3,2 -dioxaborolan-2 -yl)-1 H-indazol-3 -amine (165 mg, 0.6 mmol), N-(4 -bromopyridin-2 -yl)-2 -(4 -chlorophenyl)acetamide (162.4 mg, 0.5 mmol) and [1,1 ′-bis(di-tert-butylphosphino)ferrocene]dichloropalladium(II) catalyst (16.2 mg, 0.025 mmol) were placed in a 2-5 mL microwave vial which was sealed and purged with nitrogen for 5 mins. Ethanol (O₂ -free, 1.3 mL) was added, and the suspension stirred at 40° C. under nitrogen for a further 10 mins. K₃ PO₄ (1 M, 0.65 mL) was added, and the solution stirred at 70° C. for 24 hours. Reaction was quenched with water (5 mL) to yield a yellowish solid. Organics were extracted into EtOAc (50 mL) and adsorbed onto silica under reduced pressure. Chromatographic purification (Biotage SP4, 50 g cartridge, solvent system: EtOAc/methanol, gradient: 0 % 2 CV; 0-2 % 2 CV; 2 % 4 CV) yielded title product as a pale green powder (93 mg, 49 %) (¹ H (DMSO-d₆, 500 MHz) δ 3.76 (s, 2 H), 5.51 (s, 2 H), 7.32 (d, J=8.5 Hz, 1 H), 7.38 (m, 5 H), 7.58 (dd, J=8.6 & 1.6 Hz, 1 H), 8.13 (d, J=0.9 Hz, 1 H), 8.34 (d, J=5.3 Hz, 1 H), 8.40 (s, 1 H), 10.75 (s, 1 H), 11.55 (br s, 1 H); ¹³ C (DMSO-d₆, 125 MHz) δ 42.66, 110.58 (2 C), 115.15, 117.38, 119.65, 125.48, 127.34, 128.70 (2 C), 131.64 (2 C), 131.82, 135.27, 141.98, 148.83, 150.46, 150.61, 153.20, 170.24 . HRMS: C₂₀H₁₇ ClN₅O requires 378.1116 found 378.1114 (M+H)⁺.

### N-(4 -(3 -Amino-1 H-indazol-5 -yl)pyridin-2 -yl)-2 -(4 -(methylthio)phenyl)acetamide SU1570

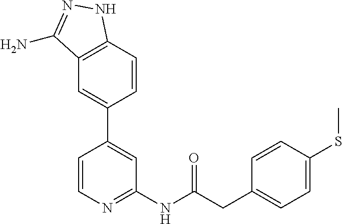 [SMILES: CSc1ccc(CC(=O)Nc2cc(-c3ccc4[nH]nc(N)c4c3)ccn2)cc1]

5 -(4,4,5,5 -Tetramethyl-1,3,2 -dioxaborolan-2 -yl)-1 H-indazol-3 -amine (110 mg, 0.42 mmol), N-(4 -chloropyridin-2 -yl)-2 -(4 -(methylthio)phenyl)acetamide (102 mg, 0.35 mmol) and [1,1 ′-bis(di-tert-butylphosphino)ferrocene]dichloropalladium(II) catalyst (23 mg, 0.035 mmol) were placed in a 2-5 mL microwave vial which was sealed and purged with nitrogen for 5 mins. Ethanol (O₂ -free, 1 mL) was added, and the suspension stirred at 40° C. under nitrogen for a further 10 mins. K₃ PO₄ (1 M, 0.5 mL) was added, and the solution stirred at 70° C. for 24 hours. Reaction was quenched with water (5 mL) to yield a yellowish solid. Supernatant was removed and solid resuspended in 80 % EtOAc/methanol (8 mL) and adsorbed onto silica under reduced pressure. Chromatographic purification (Biotage SP4, 50 g cartridge, solvent system: EtOAc/methanol, gradient: 0 % 2 CV; 0-5 % 3 CV; 5 % 4 CV) yielded title product as a brown solid (41 mg, 25 %)¹ H (DMSO-d₆, 400 MHz) δ 2.46 (s, 3 H), 3.72 (s, 2 H), 5.55 (s, 2 H), 7.24 (m, 2 H), 7.33 (m, 3 H), 7.39 (dd, J=5.3 & 1.8 Hz, 1 H), 7.59 (dd, J=8.8 & 1.8 Hz, 1 H), 8.15 (d, J=1 Hz, 1 H), 8.34 (dd, J=5.3 & 0.5 Hz, 1 H), 8.42 (s, 1 H), 10.75 (s, 1 H), 11.58 (br s, 1 H); ¹³ C (DMSO-d₆, 100 MHz) 5 15.44 (3 H), 42.92 (2 H), 110.57 (2 C), 115.14, 117.34, 119.65, 125.48, 126.65 (2 C), 127.38, 130.32 (2 C), 133.00, 136.64, 141.99, 148.81, 150.45, 150.59, 153.25, 170.57 ; HRMS: C₂₁H₂₀N₅OS requires 390.1383 found 390.1377 . (M+H)⁺

### N-(4 -(3 -Amino-1 H-indazol-5 -yl)pyridin-2 -yl)-2 -(4 -(methylsulfonyl)phenyl)acetamide SU1575

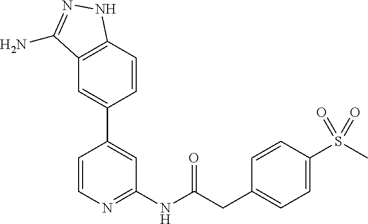 [SMILES: CS(=O)(=O)c1ccc(CC(=O)Nc2cc(-c3ccc4[nH]nc(N)c4c3)ccn2)cc1]

5 -(4,4,5,5 -Tetramethyl-1,3,2 -dioxaborolan-2 -yl)-1 H-indazol-3 -amine (118 mg, 0.45 mmol), N-(4 -chloropyridin-2 -yl)-2 -(4 -(methylsulfonyl)phenyl)acetamide (139 mg, 0.4 mmol) and [1,1 ′-bis(di-tert-butylphosphino)ferrocene]dichloropalladium(II) catalyst (23 mg, 0.035 mmol) were placed in a 2-5 mL microwave vial which was sealed and purged with nitrogen for 5 mins. Ethanol (O₂ -free, 1.12 mL) was added, and the suspension stirred at 50° C. under nitrogen for a further 10 mins. K₃ PO₄ (1 M, 0.56 mL) was added, and the solution stirred at 70° C. for 24 hours. Reaction was quenched with water (5 mL) to yield a yellowish solid. Supernatant was removed and solid resuspended in 80 % EtOAc/methanol (6 mL) and adsorbed onto silica under reduced pressure.

Chromatographic purification (Biotage SP4, 50 g cartridge, solvent system: EtOAc/methanol, gradient: 0 % 2 CV; 0-5 % 3 CV; 5 % 4 CV) yielded title product as a greenish solid (30 mg, 17 %). ¹ H (DMSO-d₆, 400 MHz) δ 3.21 (s, 3 H), 3.93 (s, 2 H), 5.52 (s, 2 H), 7.34 (d, J=8.8 Hz, 1 H), 7.41 (dd, J=5.3 & 1.8 Hz, 1 H), 7.59 (dd, J=8.7 & 1.6 Hz, 1 H), 7.64 (d, J=8.3 Hz, 2 H), 7.91 (d, J=8.4 Hz, 2 H), 8.15 (d, J=1 Hz, 1 H), 8.36 (d, J=5.5 Hz, 1 H), 8.42 (s, 1 H), 10.85 (s, 1 H), 11.57 (s, 1 H); ¹³ C (DMSO-d₆, 100 MHz) δ 43.18, 44.07, 110.61 (2 C), 115.14, 117.45, 119.66, 125.47, 127.30, 127.45 (2 C), 130.77 (2 C), 139.70, 141.99, 142.27, 148.86, 150.45, 150.63, 153.13, 169.73 ; HRMS: C₂₁H₂₀N₅O₃ S requires 422.1281 found 422.1276 . (M+H)⁺

### N-(4 -(3 -Amino-1 H-indazol-5 -yl)pyridin-2 -yl)-2 -(4 -methoxyphenyl)acetamide SU1573

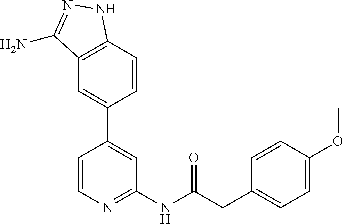 [SMILES: COc1ccc(CC(=O)Nc2cc(-c3ccc4[nH]nc(N)c4c3)ccn2)cc1]

5 -(4,4,5,5 -Tetramethyl-1,3,2 -dioxaborolan-2 -yl)-1 H-indazol-3 -amine (110 mg, 0.42 mmol), N-(4 -chloropyridin-2 -yl)-2 -(4 -methoxyphenyl)acetamide (97 mg, 0.35 mmol) and [1,1 ′-bis(di-tert-butylphosphino)ferrocene]dichloropalladium(II) catalyst (23 mg, 0.035 mmol) were placed in a 2-5 mL microwave vial which was sealed and purged with nitrogen for 5 mins. Ethanol (O₂ -free, 1 mL) was added, and the suspension stirred at 50° C. under nitrogen for a further 5 mins. K₃ PO₄ (1 M, 0.5 mL) was added, and the solution stirred at 70° C. for 24 hours. Reaction was quenched with water (5 mL) to yield a yellowish solid. Supernatant was removed and solid was resuspended in 80 % EtOAc/methanol (6 mL) and adsorbed onto silica. Chromatographic purification (Biotage SP4, 50 g cartridge, solvent system: EtOAc/methanol, gradient: 0 % 2 CV; 0-5 % 3 CV; 5 % 6 CV) yielded title product as a pale brown solid (90 mg, 69 %). ¹ H (DMSO-d₆, 400 MHz) δ 3.68 (s, 2 H), 3.73 (s, 3 H), 5.55 (br s, 2 H), 6.91 (m, 2 H), 7.29 (m, 2 H), 7.34 (d, J=8.8 Hz, 1 H), 7.39 (dd, J=5.4 & 1.6 Hz, 1 H), 7.59 (dd, J=8.8 & 1.8 Hz, 1 H), 8.15 (s, 1 H), 8.34 (d, J=6.3 Hz, 1 H), 8.43 (s, 1 H), 10.70 (s, 1 H), 11.59 (s, 1 H); ¹³ C (DMSO-d₆, 100 MHz) δ 42.62, 55.5, 110.51, 110.57, 114.20 (2 C), 115.11, 117.29, 119.66, 125.50, 127.36, 128.18, 130.73 (2 C), 141.95, 148.81, 150.56, 153.31, 158.55, 170.99 ; HRMS: For C₂₁H₂₀N₅O₂ requires 374.1612 found 374.1606 . (M+H)⁺

### N-(4 -(3 -Amino-1 H-indazol-5 -yl)pyridin-2 -yl)-2 -(4 -aminophenyl)acetamide SU1567

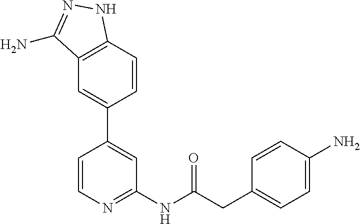 [SMILES: Nc1ccc(CC(=O)Nc2cc(-c3ccc4[nH]nc(N)c4c3)ccn2)cc1]

N-(4 -(3 -amino-1 H-indazol-5 -yl)pyridin-2 -yl)-2 -(4 -Boc-aminophenyl)acetamide (25 mg, 0.07 mmol) was dissolved in dichloromethane (3 mL), methanol (0.3 mL) and trifluoroacetic acid (1 mL) and stirred at room temperature for 24 hours. TLC showed consumption of starting material, so the reaction was quenched with 1 M Na₂ CO₃ solution (5 mL), product extracted into EtOAc (25 mL) and organics dried over magnesium sulfate, filtered and concentrated under reduced pressure to yield title compound as a brown solid (14 mg, 70 %). ¹ H (DMSO-d₆, 400MHz) δ 3.53 (s, 2 H), 4.95 (br s, 2 H), 5.55 (s, 2 H), 6.52 (d, J=8.5 Hz, 2 H), 7.02 (d, J=8.3 Hz, 2 H), 7.33 (d, J=8.8 Hz, 1 H), 7.37 (dd, J=5.3 & 1.8 Hz, 1 H), 7.58 (dd, J=8.8, 1.8 Hz, 1 H), 8.14 (d, J=1 Hz, 1 H), 8.33 (d, J=5 Hz, 1 H), 8.42 (s, 1 H), 10.55 (s, 1 H), 11.57 (s, 1 H); ¹³ C (DMSO-d₆, 150 MHz) δ 42.85, 110.51, 110.59, 114.33 (2 C), 115.10, 117.24, 119.63, 123.09, 125.51, 127.45, 130.13 (2 C), 141.99, 147.76, 148.77, 150.43, 150.54, 153.34, 171.48 HRMS: For C₂₀H₁₉N₆O requires 359.1615 found 359.1612 . (M+H)⁺

### tert-Butyl(4 -(2 -((4 -(3 -amino-1 H-indazol-5 -yl)pyridin-2 -yl)amino)-2 -oxoethyl)phenyl)carbamate SU1569

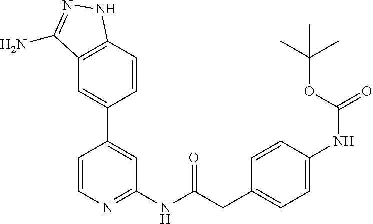 [SMILES: CC(C)(C)OC(=O)Nc1ccc(CC(=O)Nc2cc(-c3ccc4[nH]nc(N)c4c3)ccn2)cc1]

5 -(4,4,5,5 -Tetramethyl-1,3,2 -dioxaborolan-2 -yl)-1 H-indazol-3 -amine (110 mg, 0.42 mmol), N-(4 -chloropyridin-2 -yl)-2 -(4 -Boc-aminophenyl)acetamide (127 mg, 0.35 mmol) and [1,1 ′-bis(di-tert-butylphosphino)ferrocene]dichloropalladium(II) catalyst (23 mg, 0.035 mmol) were placed in a 2-5 mL microwave vial which was sealed and purged with nitrogen for 5 mins. Ethanol (O₂ -free, 1 mL) was added, and the suspension stirred at 50° C. under nitrogen for a further 10 mins. K₃ PO₄ (1 M, 0.5 mL) was added, and the solution stirred at 70° C. for 24 hours. Reaction was quenched with water (5 mL) to yield a yellowish solid. Supernatant was removed and solid resuspended in 80 % EtOAc/methanol (6 mL) and adsorbed onto silica under reduced pressure. Chromatographic purification (Biotage SP4, 50 g cartridge, solvent system: EtOAc/methanol, gradient: 0 % 2 CV; 0-5 % 3 CV; 5 % 4 CV) yielded title product as a brown solid (79 mg, 52 %) ¹ H (DMSO-d₆, 400 MHz) δ 1.46 (s, 9 H), 3.66 (s, 2 H), 5.54 (s, 2 H), 7.24 (d, J=8.3 Hz, 2 H), 7.33 (d, J=8.8 Hz, 1 H), 7.39 (m, 3 H), 7.59 (dd, J=8.3, 1.8 Hz, 1 H), 8.14 (s, 1 H), 8.33 (d, J=4.8 Hz, 1 H), 8.41 (s, 1 H), 9.29 (s, 1 H), 10.69 (s, 1 H), 11.57 (s, 1 H); ¹³ C (DMSO-d₆, 100 MHz) δ 28.60 (3 C), 42.84, 110.52, 110.56, 115.11, 117.30, 118.63, 119.66, 125.50, 127.36, 129.78, 129.90 (2 C), 138.56, 141.94, 148.81 (2 C), 150.47, 150.56, 153.25, 153.29, 170.85 HRMS: For C₂₅H₂₇N₅O₂ requires 459.2139 found 459.2133.

### N-(4 -(3 -Amino-1 H-indazol-5 -yl)pyridin-2 -yl)-2 -(2 -fluorophenyl)acetamide SU1558

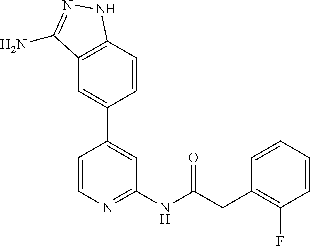 [SMILES: Nc1n[nH]c2ccc(-c3ccnc(NC(=O)Cc4ccccc4F)c3)cc12]

5 -(4,4,5,5 -Tetramethyl-1,3,2 -dioxaborolan-2 -yl)-1 H-indazol-3 -amine (85 mg, 0.325 mmol), N-(4 -chloropyridin-2 -yl)-2 -(2 -tolyl)acetamide (66 mg, 0.25 mmol) and [1,1 ′-bis(di-tert-butylphosphino)ferrocene]dichloropalladium(II) catalyst (9 mg, 0.0125 mmol) were placed in a 2-5 mL microwave vial which was sealed and purged with nitrogen for 5 mins. Ethanol (Or-free, 1 mL) was added, and the suspension stirred at 50° C. under nitrogen for a further 5 mins. K₃ PO₄ (1 M, 0.5 mL) was added, and the solution stirred at 100° C. for 24 hours. Reaction was quenched with water (8 mL) to yield a brown solid which was filtered and further washed with water (15×10 mL) and hexane (8×10 mL). Solid was resuspended in 50 % EtOAc/methanol (20 mL), filtered, and adsorbed onto silica. Chromatographic purification (Biotage SP4, 50 g cartridge, solvent system: 1 % triethylamine in EtOAc/methanol, gradient: 0 % 3 CV; 0-5 % 2 CV; 5 % 4 CV) yielded title product as a pale brown solid (7 mg, 8 %). ; ¹ H (DMSO-d₆, 400 MHz) δ 3.86 (s, 2 H), 5.55 (br s, 2 H), 7.18 (m, 2 H), 7.32 (d, J=8.8 Hz, 2 H), 7.40 (m, 2 H), 7.59 (d, J=8.3 Hz, 1 H), 8.15 (s, 1 H), 8.35 (m, 1 H), 8.43 (s, 1 H), 10.81 (s, 1 H), 11.57 (s, 1 H); ); ¹³ C (DMSO-d₆ , 150 MHz) 5 36.71, 110.56, 110.59, 115.14, 115.48 (d, J=21 Hz), 117.32, 119.63, 123.26 (d, J=16.1 Hz), 124.68 (d, J=3.4 Hz), 125.50, 127.35, 129.31 (d, J=8 Hz), 132.61 (d, J=4.6 Hz), 142.01, 148.84, 150.47, 150.58, 153.22, 161.23 (d, J=244.5 Hz), 169.58

### N-(4 -(3 -Amino-1 H-indazol-5 -yl)pyridin-2 -yl)-2 -(2 -tolyl)acetamide SU1576

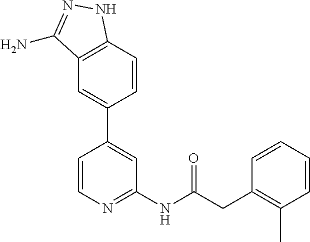 [SMILES: Cc1ccccc1CC(=O)Nc1cc(-c2ccc3[nH]nc(N)c3c2)ccn1]

5 -(4,4,5,5 -Tetramethyl-1,3,2 -dioxaborolan-2 -yl)-1 H-indazol-3 -amine (85 mg, 0.325 mmol), N-(4 -chloropyridin-2 -yl)-2 -(2 -tolyl)acetamide (65 mg, 0.25 mmol) and [1,1 ′-bis(di-tert-butylphosphino)ferrocene]dichloropalladium(II) catalyst (9 mg, 0.0125 mmol) were placed in a 2-5 mL microwave vial which was sealed and purged with nitrogen for 5 mins. Ethanol (O₂ -free, 1 mL) was added, and the suspension stirred at 50° C. under nitrogen for a further 5 mins. K₃ PO₄ (1 M, 0.5 mL) was added, and the solution stirred at 100° C. for 24 hours. Reaction was quenched with water (8 mL) to yield a brown solid which was filtered and further washed with water (15×10 mL) and hexane (8×10 mL). Solid was resuspended in 50 % EtOAc/methanol (20 mL), filtered, and adsorbed onto silica. Chromatographic purification (Biotage SP4, 50 g cartridge, solvent system: 1 % triethylamine in EtOAc/methanol, gradient: 0 % 3 CV; 0-5 % 2 CV; 5 % 4 CV) yielded title product as a pale brown solid (59 mg, 66 %). ¹ H (DMSO-d₆, 400 MHz) δ 2.32 (s, 3 H), 3.81 (s, 2 H), 5.55 (s, 2 H), 7.16 (m, 3 H), 7.28 (m, 1 H), 7.32 (d, J=8.8 Hz, 1 H), 7.39 (dd, J=5.1 & 1.5 Hz, 1 H), 7.59 (dd, J=8.8 & 1.3 Hz, 1 H), 8.14 (s, 1 H), 8.34 (d, J=5.3 Hz, 1 H), 8.43 (s, 1 H), 10.74 (s, 1 H), 11.57 (s, 1 H); 1³ C (DMSO-d₆, 150 MHz) δ 19.86, 41.20, 110.57, 110.61, 115.21, 117.28, 119.69, 125.53, 126.22, 127.20, 127.39, 130.35, 130.66, 134.98, 137.21, 141.97, 148.81, 150.49, 150.57, 153.29, 170.57 ; HRMS: For C₂₁H₁₉N₅₀ requires 358.1662 found 358.1656 . (M+H)⁺

### N-(4 -(3 -Amino-1 H-indazol-5 -yl)pyridin-2 -yl)-2 -(2 -(trifluoromethyl)phenyl)acetamide SU1564

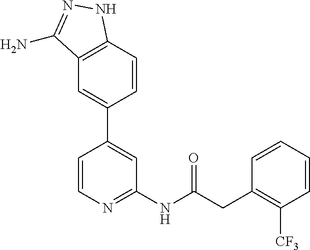 [SMILES: Nc1n[nH]c2ccc(-c3ccnc(NC(=O)Cc4ccccc4C(F)(F)F)c3)cc12]

5 -(4,4,5,5 -Tetramethyl-1,3,2 -dioxaborolan-2 -yl)-1 H-indazol-3 -amine (118 mg, 0.45 mmol), N-(4 -chloropyridin-2 -yl)-2 -(2 -(trifluoromethyl)phenyl)acetamide (126 mg, 0.4 mmol) and [1,1 ′-bis(di-tert-butylphosphino)ferrocene]dichloropalladium(II) catalyst (26 mg, 0.04 mmol) were placed in a 2-5 mL microwave vial which was sealed and purged with nitrogen for 5 mins. Ethanol (O₂ -free, 1.12 mL) was added, and the suspension stirred at 40° C. under nitrogen for a further 10 mins. K₃ PO₄ (1 M, 0.56 mL) was added, and the solution stirred at 70° C. for 24 hours. Reaction was quenched with water (5 mL) to yield a yellowish solid. Organics were extracted into EtOAc (50 mL) and adsorbed onto silica under reduced pressure. Chromatographic purification (Biotage SP4, 50 g cartridge, solvent system: EtOAc/methanol, gradient: 0 % 2 CV; 0-2 % 2 CV; 2 % 4 CV) yielded title product as a pale yellow solid (94 mg, 57 %) ¹ H (DMSO-d₆, 400 MHz) δ 4.07 (s, 2 H), 5.52 (s, 2 H), 7.33, (d, J=8.8 Hz, 1 H), 7.41 (dd, J=5.3 & 1.8 Hz, 1 H), 7.50 (t, J=7.5 Hz, 1 H), 7.56 (d, J=7.5 Hz, 1 H), 7.59 (dd, J=8.8 & 1.8 Hz, 1 H), 7.66 (t, J=7.5 Hz, 1 H), 7.72 (d, J=7.8 Hz, 1 H), 8.15 (d, J=1 Hz, 1 H), 8.36 (d, J=5.3 Hz, 1 H), 8.42 (s, 1 H), 10.80 (s, 1 H), 11.56 (br s, 1 H); ¹³ C (DMSO-d₆, 100 MHz) δ 40.12, 110.48, 110.56, 115.14, 117.24, 119.59, 124.97 (d, J=273.6 Hz), 125.47, 126.07 (d, J=5.9 Hz), 127.31, 127.81, 128.15 (d, J=30.2 Hz), 132.67, 134.11, 134.25, 142.00, 148.85, 150.46, 150.56, 153.25, 169.62 . HRMS: C₂₁H₁₇ F3N₅O requires 412.1380 found 412.1373 . (M+H)⁺

### N-(4 -(3 -Amino-1 H-indazol-5 -yl)pyridin-2 -yl)-2 -(2 -chlorophenyl)acetamide SU1559

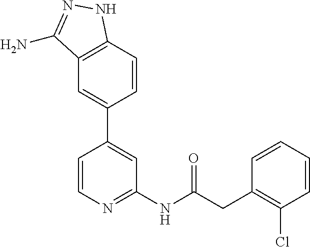 [SMILES: Nc1n[nH]c2ccc(-c3ccnc(NC(=O)Cc4ccccc4Cl)c3)cc12]

5 -(4,4,5,5 -Tetramethyl-1,3,2 -dioxaborolan-2 -yl)-1 H-indazol-3 -amine (165 mg, 0.6 mmol), N-(4 -bromopyridin-2 -yl)-2 -(2 -chlorophenyl)acetamide (162.4 mg, 0.5 mmol) and [1,1 ′-bis(di-tert-butylphosphino)ferrocene]dichloropalladium(II) catalyst (16.2 mg, 0.025 mmol) were placed in a 2-5 mL microwave vial which was sealed and purged with nitrogen for 5 mins. Ethanol (O₂ -free, 1.3 mL) was added, and the suspension stirred at 40° C. under nitrogen for a further 10 mins. K₃ PO₄ (1 M, 0.65 mL) was added, and the solution stirred at 70° C. for 24 hours. Reaction was quenched with water (5 mL) to yield a yellowish solid. Organics were extracted into EtOAc (50 mL) and adsorbed onto silica under reduced pressure. Chromatographic purification (Biotage SP4, 50 g cartridge, solvent system: EtOAc/methanol, gradient: 0 % 2 CV; 0-2 % 2 CV; 2 % 4 CV) yielded title product as a greenish solid (98 mg, 52 %) ¹ H (DMSO-de, 500 MHz) δ 3.97 (s, 2 H), 5.52 (s, 2 H), 7.31, (m, 3 H), 7.39 (dd, J=5.3 & 1.6 Hz, 1 H), 7.44 (m, 2 H), 7.58 (dd, J=8.6 & 1.7 Hz, 1 H), 8.14 (d, J=1.3 Hz, 1 H), 8.34 (d, J=5.3 Hz, 1 H), 8.43 (s, 1 H), 10.78 (s, 1 H), 11.55 (br s, 1 H); ¹³ C (DMSO-d₆, 125 MHz) δ 41.15, 110.52, 110.56, 115.14, 117.25, 119.60, 124.48, 127.33, 127.51, 129.10, 129.46, 132.83, 134.25, 134.30, 142.01, 148.84, 150.46, 150.55, 153.26, 169.46 . HRMS: C₂₀H₁₇ ClN₅O requires 378.1116 found 378.1114 (M+H)⁺.

### N-(4 -(3 -Amino-1 H-indazol-5 -yl)pyridin-2 -yl)-2 -(2 -methoxyphenyl)acetamide SU1571

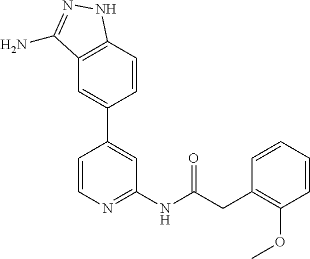 [SMILES: COc1ccccc1CC(=O)Nc1cc(-c2ccc3[nH]nc(N)c3c2)ccn1]

5 -(4,4,5,5 -Tetramethyl-1,3,2 -dioxaborolan-2 -yl)-1 H-indazol-3 -amine (110 mg, 0.42 mmol), N-(4 -chloropyridin-2 -yl)-2 -(2 -methoxyphenyl)acetamide (97 mg, 0.35 mmol) and [1,1 ′-bis(di-tert-butylphosphino)ferrocene]dichloropalladium(II) catalyst (23 mg, 0.035 mmol) were placed in a 2-5 mL microwave vial which was sealed and purged with nitrogen for 5 mins. Ethanol (O₂ -free, 1 mL) was added, and the suspension stirred at 50° C. under nitrogen for a further 5 mins. K₃ PO₄ (1 M, 0.5 mL) was added, and the solution stirred at 70° C. for 24 hours. Reaction was quenched with water (5 mL) to yield a yellowish solid. Supernatant was removed and solid was resuspended in 80 % EtOAc/methanol (6 mL) and adsorbed onto silica. Chromatographic purification (Biotage SP4, 50 g cartridge, solvent system: EtOAc/methanol, gradient: 0 % 2 CV; 0-5 % 3 CV; 5 % 6 CV) yielded title product as a pale brown solid (99 mg, 76 %). ¹ H (DMSO-d₆, 400 MHz) δ 3.76 (s, 2 H), 3.77 (s, 3 H), 5.54 (br s, 2 H), 6.91 (t, J=7.3 Hz, 1 H), 6.99 (d, J=7.9 Hz, 1 H), 7.24 (d, J=7.5 Hz, 2 H), 7.32 (d, J=8.3 Hz, 1 H), 7.32 (dd, J=5.3 & 1.8 Hz, 1 H), 7.59 (d, J=8.8 Hz 1 H), 8.15 (s, 1 H), 8.34 (d, J=5.7 Hz, 1 H), 8.44 (s, 1 H), 10.55 (s, 1 H), 11.57 (s, 1 H); ¹³ C (DMSO-d₆, 100 MHz) δ 38.12, 55.89, 110.43, 110.54, 111.14, 115.11, 117.10, 119.61, 120.62, 124.44, 125.48, 127.36, 128.60, 131.56, 141.95, 148.81, 150.47, 153.36, 157.78, 170.61 ; HRMS: For C₂₁H₂₀N₅O₂ requires 374.1612 found 374.1606 . (M+H)⁺

### N-(4 -(3 -Amino-1 H-indazol-5 -yl)pyridin-2 -yl)-2 -(2 -pyridyl)acetamide SU1562

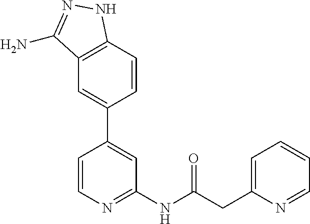 [SMILES: Nc1n[nH]c2ccc(-c3ccnc(NC(=O)Cc4ccccn4)c3)cc12]

N-(4 -Chloropyridin-2 -yl)-2 -(2 -pyridyl)acetamide (100 mg, 0.4 mmol), 5 -(4,4,5,5 -Tetramethyl-1,3,2 -dioxaborolan-2 -yl)-1 H-indazol-3 -amine (118 mg, 0.45 mmol), and [1,1 ′-bis(di-tert-butylphosphino)ferrocene]dichloropalladium(II) catalyst (26 mg, 0.04 mmol) were placed in a 2 -5 mL microwave vial which was sealed and purged with nitrogen for 5 mins. Ethanol (O₂ -free, 1.12 mL) was added, and the suspension stirred under nitrogen for a further 10 mins. K₃ PO₄ (O₂ -free, 1 M, 0.56 mL) was added, and the solution stirred at 70° C. for 24 hours. Organics were extracted into EtOAc/methanol 9:1 (50 mL), washed with water and dried over magnesium sulfate, then adsorbed onto silica under reduced pressure. Chromatographic purification (Biotage SP4, 50 g cartridge, solvent system: EtOAc/methanol/1 % Net₃, gradient: 0 % 5 CV; 0-2 % 2 CV; 2 % 4 CV) followed by HPLC purification yielded title compound as a bright yellow solid (as TFA salt) (42 mg, 23 %)¹ H (DMSO-d₆, 400 MHz) δ 4.23 (2, 2 H), 7.47 (m, 2 H), 7.68 (m, 1 H), 7.78 (m, 2 H), 8.22 (t, J=7.8 Hz, 1 H), 8.29 (s, 1 H), 8.41 (m, 2 H), 8.76 (d, J=5.3 Hz, 1 H), 11.06 (br. s, 1H); ¹⁹ F (DMSO-d₆, 376 MHz) δ -74.67; ¹³ C (DMSO-d₆, 100 MHz) δ 42.96, 110.72, 111.61, 117.63, 120.26, 124.52, 127.44, 127.95, 128.87, 142.24, 142.70, 145.26, 148.72, 150.28, 152.78, 152.93, 158.69, 159.04, 168.38 ; HRMS: C₁₉H₁₇N₆O requires 345.1458 found 345.1458 . (M+H)⁺

### N-(4 -(3 -Amino-1 H-indazol-5 -yl)pyridin-2 -yl)-2 -(3 -pyridyl)acetamide SU1563

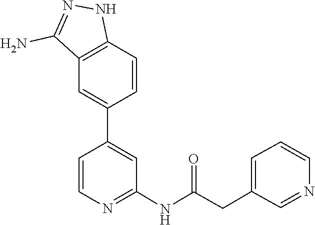 [SMILES: Nc1n[nH]c2ccc(-c3ccnc(NC(=O)Cc4cccnc4)c3)cc12]

N-(4 -Chloropyridin-2 -yl)-2 -(2 -pyridyl)acetamide (100 mg, 0.4 mmol), 5 -(4,4,5,5 -Tetramethyl-1,3,2 -dioxaborolan-2 -yl)-1 H-indazol-3 -amine (118 mg, 0.45 mmol), and [1,1 ′-bis(di-tert-butylphosphino)ferrocene]dichloropalladium(II) catalyst (26 mg, 0.04 mmol) were placed in a 2 -5 mL microwave vial which was sealed and purged with nitrogen for 5 mins. Ethanol (O₂ -free, 1.12 mL) was added, and the suspension stirred under nitrogen for a further 10 mins. K₃ PO₄ (O₂ -free, 1 M, 0.56 mL) was added, and the solution stirred at 70° C. for 24 hours. Organics were extracted into EtOAc/methanol 9:1 (50 mL), washed with water and dried over magnesium sulfate, then adsorbed onto silica under reduced pressure. Chromatographic purification (Biotage SP4, 50 g cartridge, solvent system: EtOAc/methanol/1 % Net₃, gradient: 0 % 5 CV; 0-2 % 2 CV; 2 % 4 CV) followed by HPLC purification yielded title compound as a bright yellow solid (as TFA salt) (87 mg, 47 %)¹ H (DMSO-d₆, 400 MHz) δ 4.08 (2, 2 H), 7.46 (m, 2 H), 7.76 (dd, J=8.8 & 1.8 Hz, 1 H), 7.92 (dd, J=7.8 & 5.5 Hz, 1 H), 8.29 (d, J=0.8 Hz, 1 H), 8.40 (m, 3 H), 8.79 (d, J=5 Hz, 1 H), 8.86 (s, 1 H), 11.08 (s, 1 H); ¹⁹ F (DMSO-d₆, 376 MHz) δ -74.61; ¹³ C (DMSO-d₆, 100 MHz) δ 49.05, 110.72, 111.56, 117.56, 120.22, 127.84, 128.83, 134.89, 142.22, 142.75, 144.97, 145.25, 148.73, 150.24, 152.90, 158.77, 159.12, 169.29 ; HRMS: C₁₉H₁₇N₆O requires 345.1458 found 345.1458 . (M+H)⁺

### N-(4 -(3 -amino-1 H-indazol-5 -yl)pyridine-2 -yl)-2 -(pyridine-4 -yl)acetamide SU1286

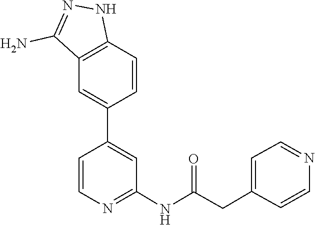 [SMILES: Nc1n[nH]c2ccc(-c3ccnc(NC(=O)Cc4ccncc4)c3)cc12]

A suspension of N-(4 -bromopyridin-2 -yl)-2 -(pyridin-4 -yl)acetamide (0.100 g, 0.35 mmol), 5 -(4,4,5,5 -Tetramethyl-1,3,2 -dioxaborolan-2 -yl)-1 H-indazol-3 -amine (0.136 g, 0.53 mmol), tetrakis(triphenylphosphine)palladium(0 ) catalyst (0.020 g) in IPA/H₂O (2:1 mL) was degassed with nitrogen. Potassium phosphate monobasic (0.180 g, 0.88 mmol) was added and the mixture was heated in the microwave at 160° C. for 40 min. The reaction mixture was concentrated and extracted with EtOAc (3×10 mL) in H₂O (10 mL). The organic fractions were combined, dried over anhydrous MgSO₄, filtered, and concentrated under reduced pressure to give the crude product. Purification by flash column chromatography (silica gel with EtOAc) afforded the title compound as an off-white solid (0.022 g, 0.06 mmol, 18 %). ¹H NMR (500 MHz, DMSO-d₆ ): δ 3.97 (s, 2 H), 5,55 (s, 2 H), 7.27 (m, 2 H), 7.38 (m, 2 H), 7.58 (dd, J=7.7, and 1.81 Hz, 1 H), 7.76 (td, J=7.7, and 1.80 Hz, 1 H), 8.14 (s, 1 H), 8.31 (d, J=5.3 Hz, 1 H), 8.43 (s, 1 H), 8.51 (d, J=4.05 Hz, 1 H), 10.75 (s, 1 H), 11.55 (s, 1 H). LRMS: C₁₉H₁₆N₆O requires 344.14, found 345.20 (M+H)⁺.

### N-(4 -(3 -Amino-1 H-indazol-5 -yl)pyridin-2 -yl)-3 -phenylpropanamide SU1579

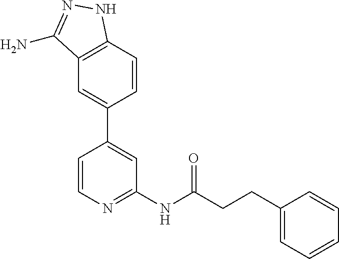 [SMILES: Nc1n[nH]c2ccc(-c3ccnc(NC(=O)CCc4ccccc4)c3)cc12]

5 -(4,4,5,5 -Tetramethyl-1,3,2 -dioxaborolan-2 -yl)-1 H-indazol-3 -amine (43 mg, 0.17 mmol), N-(4 -chloropyridin-2 -yl)-3 -phenylpropanamide (37 mg, 0.14 mmol) in ethanol (O₂ -free, 0.4 mL) and [1,1 ′-bis(di-tert-butylphosphino)ferrocene]dichloropalladium(II) catalyst (9 mg, 0.017 mmol) were placed in a 2-5 mL microwave vial which was sealed and purged with nitrogen for 5 mins.

The suspension stirred at 50° C. under nitrogen for a further 5 mins. K₃ PO₄ (1 M, 0.2 mL) was added, and the solution stirred at 70° C. for 24 hours. Reaction was quenched with water (5 mL) and precipitate was filtered, washed with water (10×5 mL) and hexane (5×5 mL), dissolved in EtOAc and adsorbed onto silica under reduced pressure. Chromatographic purification (Biotage SP4, 50 g cartridge, solvent system: EtOAc/methanol, 0 % 2 CV; 0-5 % 3 CV; 5 % 4 CV) yielded title product as a grey/green solid (27 mg, 54 %). ¹ H (DMSO-d₆, 400 MHz) δ 2.76 (t, J=7.3 Hz, 2 H), 2.94 (t, J=7.8 Hz, 2 H), 5.55 (s, 2 H), 7.19 (m, 1 H), 7.29 (m, 4 H), 7.37 (m, 2 H), 7.60 (dd, J=8.8, 1.5 Hz, 1 H), 8.17 (s, 1 H), 8.32 (d, J=5.3 Hz, 1 H), 8.46 (s, 1 H), 10.51 (s, 1 H), 11.59 (br s, 1 H); ¹³ C (DMSO-d₆, 100 MHz) δ 31.10, 38.10, 110.63, 115.16, 117.18, 119.66, 125.51, 126.42, 127.47, 128.75 (2 C), 128.79 (2 C), 141.59, 142.00, 148.75, 150.46, 150.56, 153.28, 171.92 ; HRMS (ESI+ve): For C₂₁H₁₉N₅O requires 358.1662 found 358.1659 . (M+H)⁺

### Aryl Substituents at Position 2 of the Aminopyridine Ring

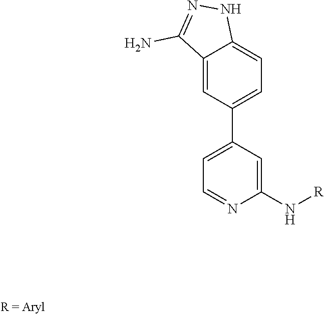

### 5 -(2 -(Phenylamino)pyridin-4 -yl)-1 H-indazol-3 -amine SU1140

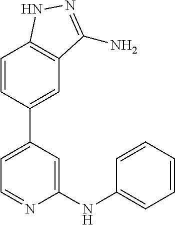 [SMILES: Nc1n[nH]c2ccc(-c3ccnc(Nc4ccccc4)c3)cc12]

A 0.5-2 mL microwave vial was charged with 5 -(2 -chloropyridin-4 -yl)-1 H-indazol-3 -amine (SU1118 ) (0.040 g, 0.16 mmol), aniline (446 μL, 4.89 mmol), p-toluenesulfonic acid (0.031 g, 0.16 mmol) and 1,4 -dioxane (554 μL, mmol), was sealed with an aluminium crimp cap fitted with a disposable PTFE/silicon septum and purged with nitrogen. The reaction mixture was heated to 210° C. under microwave irradiation for 50 min. The reaction mixture was then cooled to 40° C. and diluted by dropwise addition of Et₂O (7 mL) via syringe. The mixture was stirred for 30 min and allowed to cool down to room temperature. The cap was then removed and a solid was filtered, washed with Et₂O (2×7 mL), triturated with a 2 % aqueous triethylamine (3×7 mL), washed with H₂O (2×7 mL) and Et₂O (1 mL). The solid was dried to afford the title compound as an ecru solid (0.042 g, 0.13 mmol, 86 %). ¹H NMR (400 MHz, DMSO-d₆ ): δ 5.51 (s, 2 H), 6.88 (t, J=7.4 Hz, 1 H), 7.04 (dd, J=5.5, 1.5 Hz, 1 H), 7.10 (s, 1 H), 7.27 (t, J=7.4 Hz, 2 H), 7.34 (d, J=8.6 Hz, 1 H), 7.57 (d, J=8.7 Hz, 1 H), 7.71 (d, J=7.6 Hz, 2 H), 8.12 (s, 1 H), 8.19 (d, J=5.4 Hz, 1 H), 9.08 (s, 1 H), 11.56 (br s, 1 H). HRMS: Calculated for C₁₈H₁₆N₅ requires 302.14002 found 302.13918 (M+H)⁺.

### 5 -(2 -{[3 -Propan-2 yl)phenyl]amino}pyridine-4 -yl)-1 H-indazol-3 -amine SU1200

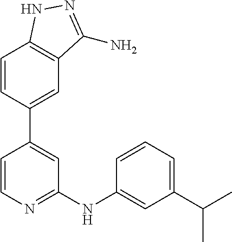 [SMILES: CC(C)c1cccc(Nc2cc(-c3ccc4[nH]nc(N)c4c3)ccn2)c1]

5 -(2 -Chloropyridin-4 -yl)-1 H-indazol-3 -amine (SU1118 ) (0.053 g, 0.22 mmol), 3 -isopropylaniline
(160 L, 1.14 mmol), p-toluenesulfonic acid (0.004 g, 0.02 mmol) in 1,4 dioxane (0.8 mL) were heated in the microwave at 210° C. for 50 min. The reaction mixture was then cooled to room temperature and diluted with Et₂O (7 mL). The resulting solid was filtered under reduced pressure and washed with Et₂O (2×7 mL) and suspended in H₂O (5 mL) to which 10 % NH₃ (aqueous, 0.5 mL) was added. The resulting pH neutral solution was filtered under reduced pressure, washed with H₂O and dried to afford the title compound as an off white solid (0.012 g, 0.04 mmol, 16 %). ¹H NMR (DMSO-d₆ ): δ 1.22 (d, J=7.2 Hz, 6 H), 2.81-2.88 (m, 1 H), 5.51 (br s, 2 H), 6.78 (d, J=7.6 Hz, 1 H), 7.03 (d, J=5.2 Hz, 1 H), 7.10 (s, 1 H), 7.18 (t, J=15.6 Hz, 1 H), 7.34 (d, J=8.8 Hz, 1 H), 7.48 (br s, 1 H), 7.56 (d, J=8.8 Hz, 1 H), 7.62 (d, J=8.8 Hz, 1 H), 8.12 (s, 1 H), 8.19 (d, J=5.6 Hz, 1 H), 9.01 (s, 1 H), 11.47 (br s, 1 H), HRMS: For C₂₁H₂₂N₅ requires 344.1867, found 344.1870 . (M+H)⁺

### 3 -((4 -(3 -Amino-1 H-indazol-5 -yl)pyridin-2 -yl)amino)phenol SU1151

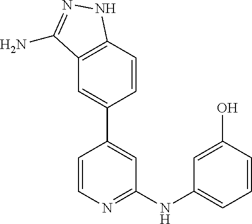 [SMILES: Nc1n[nH]c2ccc(-c3ccnc(Nc4cccc(O)c4)c3)cc12]

A 0.5-2 mL microwave vial was charged with 5 -(2 -chloropyridin-4 -yl)-1 H-indazol-3 -amine (SU1118 ) (0.037 g, 0.15 mmol), 3 -aminophenol (0.330 g, 3.02 mmol), p-toluenesulfonic acid (0.003 g, 0.02 mmol) and 1,4 -dioxane (0.5 mL) was sealed with an aluminium crimp cap fitted with a disposable PTFE/silicon septum and purged with nitrogen. The reaction mixture was heated to 210° C. under microwave irradiation for 50 min. The reaction mixture was then cooled to 40° C. and diluted by dropwise addition of Et₂O (7 mL) via syringe. The mixture was stirred for 30 min and allowed to cool to room temperature. The cap was then removed and a solid was filtered, washed with Et₂O (2×7 mL) and suspended in H₂O (5 mL). To this suspension was added 10 % aqueous NH₃ (0.5 mL) and the mixture was heated to 35° C. at 800 mbar until the pH was neutral. The suspension was then filtered and washed with H₂O (3×7 mL). The solid was dried to afford the title compound as a brown solid (0.005 g, 0.02 mmol, 10 %). ¹ H NMR (400 MHz, DMSO-d₆): 6 5.50 (s, 2 H), 6.27-6.33 (m, 1 H), 6.98-7.13 (m, 4 H), 7.27 -7.37 (m, 2 H), 7.56 (d, J=8.7 Hz, 1 H), 8.11 (s, 1 H), 8.18 (d, J=5.4 Hz, 1 H), 8.95 (s, 1 H), 9.19 (br s, 1 H), 11.56 (br s, 1 H). HRMS: Calculated for C₁₈H₁₆N₅O requires 318.13494 found 318.13451 (M+H)⁺.

### (3 -((4 -(3 -Amino-1 H-indazol-5 -yl)pyridin-2 -yl)amino)phenyl)methanol SU1156

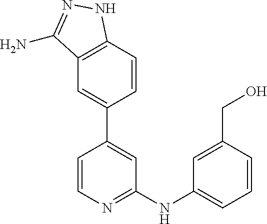 [SMILES: Nc1n[nH]c2ccc(-c3ccnc(Nc4cccc(CO)c4)c3)cc12]

A 0.5-2 mL microwave vial was charged with 5 -(2 -chloropyridin-4 -yl)-1 H-indazol-3 -amine (SU1118 ) (0.035 g, 0.143 mmol, 1 eq.), (3 -aminophenyl)methanol (0.176 g, 1.43 mmol, 10 eq.), p-toluenesulfonic acid (0.003 g, 0.01 mmol, 0.1 eq.) and 1,4 -dioxane (0.5 mL), was sealed with an aluminium crimp cap fitted with a disposable PTFE/silicon septum, and purged with nitrogen.

The reaction mixture was heated to 210° C. under microwave irradiation for 50 minutes. The reaction mixture was then cooled to ca. 40° C. and diluted by dropwise addition of Et₂O (7 mL) via syringe. The mixture was stirred and sonicated for 30 minutes and allowed to cool to room temperature. The cap was then removed and a solid was filtered, washed with Et₂O (2×7 mL), suspended in 10 % aqueous NH₃ (5 mL) and sonicated. The suspension was then filtered to give a crude product (ca. 23 mg), which was purified by HPLC to afford the title compound (0.007 g, 0.02 mmol, 11 %) ¹H NMR (DMSO-D6 ) 5: 4.52 (s, 2 H), 7.11-7.02 (m, 1 H), 7.26 -7.17 (m, 2 H), 7.38-7.31 (m, 1 H), 7.40 (d, J=8.7 Hz, 1 H), 7.53-7.44 (m, 2 H), 7.65 (d, J=8.9Hz, 1 H), 8.12 (d, J=5.8 Hz, 1 H), 8.25 (s, 1 H), HRMS: Calculated for C₁₉H₁₈0N₅ (M+H⁺): 332.1506 ; Found: 332.1503

### 3 -((4 -(3 -Amino-1 H-indazol-5 -yl)pyridin-2 -yl)amino)benzoic acid SU1255

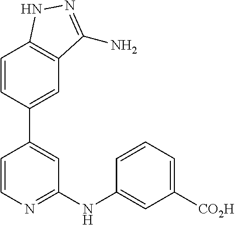 [SMILES: Nc1n[nH]c2ccc(-c3ccnc(Nc4cccc(C(=O)O)c4)c3)cc12]

To a suspension of 3 -((4 -chloropyridin-2 -yl)amino)benzoic acid (0.051 g, 0.23 mmol), 5 -(4,4,5,5 -tetramethyl-1,3,2 -dioxaborolan-2 -yl)-1 H-indazol-3 -amine (0.127 g, 0.49 mmol) and

′-bis(di-tert-butylphosphino)ferrocene]dichloropalladium(II) catalyst (0.022 g, 0.034 mmol) in EtOH (1.6 mL, degassed under nitrogen) was added K₃ PO₄ (1 M, 0.8 mL) and the reaction mixture was heated to 100° C. for 20 h. The reaction mixture was cooled to room temperature and water (7 mL) was added and the reaction was stirred at room temperature for 5 mins. The reaction mixture was filtered and the resulting solid was washed with water (10 mL), hexane (20 mL), EtOAc (10 mL) and MeCOH (8 mL) and then dried. The solid was dissolved in DMF (2 mL) and purified by HPLC (t_{R}=11 min) to afford the title compound as the TFA salt (0.0076 g, 0.022 mmol, 10.8 %). ¹H NMR (DMSO-d₆ ): δ 7.17 (m, 2 H), 7.43 (q, J=7.5 Hz, 2 H), 7.55 (d, J=7.6 Hz, 1 H), 7.66 (dd, J=1.6, 8.8 Hzm 1 H), 7.95 (d, J=8.4 Hz, 1 H), 8.22 (m, 3 H), 9.60 (br s, 1 H), 11.97 (br s, 1 H), 12.90 (br s, 1 H). HRMS: For C₁₉H₁₆02N₅ requires 346.1299 found 346.1295 . (M+H)⁺

### Ethyl 3 -((4 -(3 -amino-1 H-indazol-5 -yl)pyridin-2 -yl)amino)benzoate SU1239

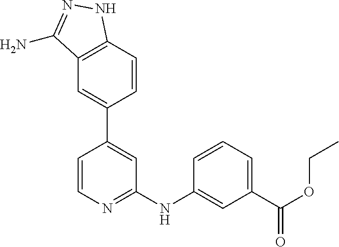 [SMILES: CCOC(=O)c1cccc(Nc2cc(-c3ccc4[nH]nc(N)c4c3)ccn2)c1]

A 2-5 mL microwave vial was charged with 4 ethyl 3 -((4 -chloropyridin-2 -yl)amino)benzoate (0.230 g, 0.83 mmol, 1 eq), 5 -(4,4,5,5 -tetramethyl-1,3,2 -dioxaborolan-2 -yl)-1 H-indazol-3 -amine (0.301 g, 1.16 mmol, 1.4 eq.), [1,1 ′-bis(di-tert-butylphosphino)ferrocene]dichloropalladium(II) catalyst (0.038 g, 0.06 mmol, 7 mol %), sealed with an aluminium crimp cap fitted with a disposable PTFE/silicon septum, and purged with nitrogen. Ethanol (2.4 mL) was added, and the mixture was stirred under nitrogen for some minutes before adding K₃ PO₄ (1.2 mL, 1 M in water, O₂ -free). The mixture was heated to ca. 50° C. and purged with nitrogen for 10 minutes. The resulting mixture was then left stirring at 70° C. for 13 hours, during which time the initial clear orange solution became cloudy. The reaction mixture was then stirred at room temperature and diluted by dropwise addition of water (ca. 7 mL) to give a suspension that was stirred for some hours before being filtered. The filtered solid was washed with water (7 mL×2 ), hexane (7 mL×2 ) and EtOAc (7 mL), and purified by flash chromatography (Biotage SP4, 50 g SiO₄, EtOAc) to afford the title compound as an off-white solid (0.095 g, 0.26 mmol, 31 %), ¹ H NMR (DMSO-D6 ) 6: 1.34 (t, J=7.1 Hz, 3 H), 4.32 (q, J=7.0 Hz, 2 H), 5.52 (s, 2 H), 7.14 -7.07 (m, 2 H), 7.35 (d, J=8.7 Hz, 1 H), 7.41 (t, J=7.9 Hz, 1 H), 7.48 (d, J=6.8 Hz, 1 H), 7.58 (d, J=8.8 Hz, 1 H), 8.09 (d, J=8.1 Hz, 1 H), 8.14 (s, 1 H), 8.23 (d, J=5.3 Hz, 1 H), 8.31 (s, 1 H), 9.38 (s, 1 H), 11.58 (s, 1 H), HRMS: Calculated for C₂—H₂ oO₂N₅ (M+H⁺):374.1612 ; Found: 374.1633

### 3 -((4 -(3 -Amino-1 H-indazol-5 -yl)pyridin-2 -yl)amino)benzamide SU1248

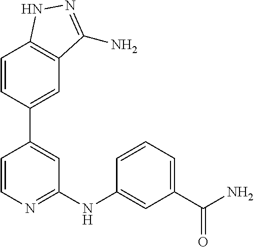 [SMILES: NC(=O)c1cccc(Nc2cc(-c3ccc4[nH]nc(N)c4c3)ccn2)c1]

5 -(2 -Chloropyridin-4 -yl)-1 H-indazol-3 -amine (SU1118 ) (0.073 g, 0.3 mmol), 3 -aminobenzamide (0.247 g, 1.8 mmol), p-toluenesulfonic acid (0.009 g, 0.047 mmol) in 1,4 dioxane (2.5 mL) were heated in the microwave at 210° C. for 50 min. The reaction mixture was then cooled to room temperature and diluted with Et₂O (7 mL). The resulting solid was filtered under reduced pressure and washed with Et₂O (30 mL), 2 % NEt₃ (aqueous, 20 mL), was added. The resulting pH neutral solution was filtered under reduced pressure, washed with H₂O (20 mL), Et₂O (30 mL) and dried. The organics were concentrated under reduced pressure and the resulting residue was dissolved in DMF (2 mL) and purified by HPLC (t_{R}=8 min) to afford the title compound as the TFA salt (0.0028 g, 0.008 mmol, 2.7 %). ¹H NMR (DMSO-de): 5 7.19 (m, 2 H), 7.33 (br s, 1 H), 7.41 (m, 2 H), 7.50 (m, 1 H), 7.65 (dd, J=1.6, 8.8 Hz, 1 H), 7.85 (d, J=7.2 Hz, 1 H), 7.91 (s, 1 H), 8.06 (s, 1 H), 8.19 (d, J=6.0 Hz, 1 H), 8.23 (s, 1 H), 9.57 (br s, 1 H), 11.82 (br s, 1 H). HRMS: For C₁₉H₁₇0N₆ requires 345.1458 found 345.1460 . (M+H)⁺

### 3 -((4 -(3 Aamino-1 H-indazol-5 -yl)pyridine-2 -yl)amino)-N-(2 -hydroxyethyl)benzamide SU1243

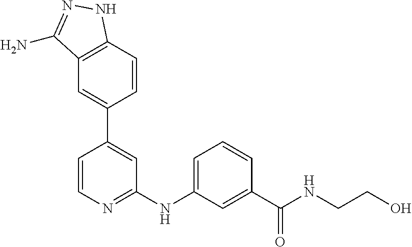 [SMILES: Nc1n[nH]c2ccc(-c3ccnc(Nc4cccc(C(=O)NCCO)c4)c3)cc12]

A 0.5-2 mL microwave vial was charged with ethyl 3 -((4 -(3 -amino-1 H-indazol-5 -yl)pyridin-2 -y1 )amino)benzoate (0.043 g, 0.115 mmol, 1 eq.), 2 -aminoethanol (0.211 g, 208 μL, 3.45 mmol, 30 eq.) and ethanol (350 μL), sealed with an aluminium crimp cap fitted with a disposable PTFE/silicon septum, and purged with nitrogen. The reaction mixture was heated to 170° C. under microwave irradiation for 1 h, then cooled to room temperature, diluted with water (7 mL) and allowed to stand at 4° C. for 16 hours. The precipitate was filtered, washed with cold water (7 mL×2 ) and dried to afford the title compound (0.026 g 0.067 mmol, 58 %) as a brown solid. 1 H NMR (400 MHz, DMSO-d₆ ) δ 3.52 (app q, J=6.1 Hz, 2 H), 4.72 (t, J=5.6 Hz, 1 H), 5.51 (s, 2 H), 7.07 (d, J=5.4 Hz, 1 H), 7.11 (s, 1 H), 7.29-7.39 (m, 3 H), 7.57 (d, J=8.8 Hz, 1 H), 7.91 -8.00 (m, 1 H), 8.08 (s, 1 H), 8.13 (s, 1 H), 8.21 (d, J=5.3 Hz, 1 H), 8.32 (t, J=5.4 Hz, 1 H), 9.25 (s, 1 H), 11.57 (s, 1 H). HRMS: Calculated for C₂₁H₂₁O₂N₆ (M+H⁺): 389.1721 ; Found: 389.1702

### N¹ -(4 -(3 -Amino-1 H-indazol-5 -yl)pyridin-2 -yl)benzene-1,3 -diamine SU1250

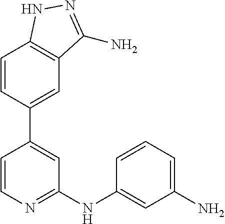 [SMILES: Nc1cccc(Nc2cc(-c3ccc4[nH]nc(N)c4c3)ccn2)c1]

5 -(2 -Chloropyridin-4 -yl)-1 H-indazol-3 -amine (SU1118 ) (0.063 g, 0.26 mmol), 3 -phenylenediamine (0.152 g, 1.41 mmol), p-toluenesulfonic acid (0.009 g, 0.047 mmol) in 1,4 dioxane (3.0 mL) were heated in the microwave at 210° C. for 50 min. The reaction mixture was then cooled to room temperature and diluted with Et₂O (7 mL). The resulting solid was filtered under reduced pressure and washed with Et₂O (30 mL), 2 % NEt₃ (aqueous, 20 mL), was added. The resulting pH neutral solution was filtered under reduced pressure, washed with H₂O (20 mL), Et₂O (30 mL) and dried. The organics were concentrated under reduced pressure and the resulting residue was dissolved in DMF (2 mL) and purified by HPLC (t_{R}=7min) to afford the title compound as the TFA salt (0.0219 g, 0.069 mmol, 26.8 %). ¹ H NMR (DMSO-d₆ ): δ 6.70 (d, J=7.2 Hz, 1 H), 7.10 (d, J=7.6 Hz, 1 H), 7.21 (m, 2 H), 7.28 (t, J=8.0 Hz, 1 H), 7.41 (d, J=8.8 Hz, 1 H), 7.47 (d, J=8.0 Hz, 1 H), 7.66 (dd, J=1.6, 8.8 Hz, 1 H), 8.15 (d, J=6.0 Hz, 1 H), 8.25 (s, 1 H), 9.78 (br s, 1 H). HRMS: For C₁₈H₁₇N₆ requires 317.1509 found 317.1504 . (M+H)⁺

### N-(3 -((4 -(3 -Amino-1 H-indazol-5 -yl)pyridin-2 -yl)amino)phenyl)acetamide SU1249

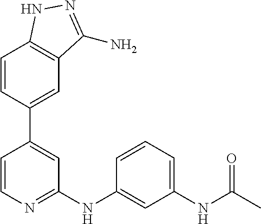 [SMILES: CC(=O)Nc1cccc(Nc2cc(-c3ccc4[nH]nc(N)c4c3)ccn2)c1]

5 -(2 -Chloropyridin-4 -yl)-1 H-indazol-3 -amine (SU1118 ) (0.063 g, 0.26 mmol), 3 -aminoacetanilide (0.209 g, 1.39 mmol), p-toluenesulfonic acid (0.009 g, 0.047 mmol) in 1,4 dioxane (3.0 mL) were heated in the microwave at 210° C. for 50 min. The reaction mixture was then cooled to room temperature and diluted with Et₂O (7 mL). The resulting solid was filtered under reduced pressure and washed with Et₂O (30 mL), 2 % NEt₃ (aqueous, 20 mL), was added. The resulting pH neutral solution was filtered under reduced pressure, washed with H₂O (20 ML), Et₂O (30 mL) and dried. The organics were concentrated under reduced pressure and the resulting residue was dissolved in DMF (2 mL) and purified by HPLC (t_{R}=10 min) to afford the title compound as the TFA salt (0.0052 g, 0.014 mmol, 5.6 %). ¹ H NMR (DMSO-d₆ ): δ 2.07 (s, 3 H), 7.20 (m, 2 H), 7.28 (m, 2 H), 7.40 (d, J=8.8 Hz, 1 H), 7.67 (dd, J=1.6, 8.8 Hz, 1 H), 7.91 (s, 1 H), 8.15 (d, J=5.9 Hz, 1 H), 8.28 (s, 1 H), 9.99 (br s, 1 H), 11.58 (br s, 1 H). HRMS: For C₂₀H₁₉ON₆ requires 359.1615 found 359.1613 . (M+H)⁺

### N-(3 -((4 -(3 -Amino-1 H-indazol-5 -yl)pyridin-2 yl)amino)phenyl)benzamide SU1260

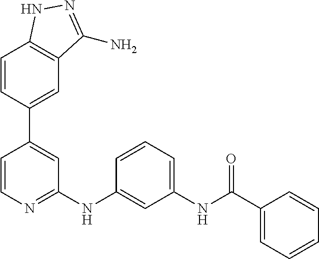 [SMILES: Nc1n[nH]c2ccc(-c3ccnc(Nc4cccc(NC(=O)c5ccccc5)c4)c3)cc12]

5 -(2 -Chloropyridin-4 -yl)-1 H-indazol-3 -amine (SU1118 ) (0.069 g, 0.28 mmol), N-(3 -aminophenyl)benzamide (0.301 g, 1.42 mmol), p-toluenesulfonic acid (0.009 g, 0.047 mmol) in 1,4 dioxane (3.0 mL) were heated in the microwave at 210° C. for 50 min. The reaction mixture was then cooled to room temperature and diluted with Et₂O (7 mL). The resulting solid was filtered under reduced pressure and washed with Et₂O (30 mL), 2 % NEt₃ (aqueous, 20 mL), was added. The resulting pH neutral solution was filtered under reduced pressure, washed with H₂O (20 mL), Et₂O (30 mL) and dried. The organics were concentrated under reduced pressure and the resulting residue was dissolved in DMF (2 mL) and purified by HPLC (t_{R}=15 min) to afford the title compound as the TFA salt (0.003 g, 0.007 mmol, 2.6 %). ⁴ H NMR (DMSO-d₆ ): δ 7.17 (s, 1 H), 7.27 (s, 1 H), 7.34 (d, J=7.2 Hz, 1 H), 7.39 (m, 3 H), 7.53 (m, 3 H), 7.57 (m, 1 H) 7.67 (d, J=7.2 Hz, 1 H), 7.96 (m, 2 H), 8.08 (s, 1 H), 8.16 (d, J=6.0 Hz, 1 H), 8.25 (s, 1 H), 10.31 (br s, 1 H), 11.84 (br s, 1 H). HRMS: For C₂₅H₂₁0N₆ requires 421.1771 found 421.1772 . (M+H)^{t}

### 5 -(2 -((3 -Phenoxyphenyl)amino)pyridin-4 -yl)-1 H-indazol-3 -amine SU1236

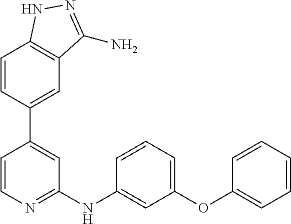 [SMILES: Nc1n[nH]c2ccc(-c3ccnc(Nc4cccc(Oc5ccccc5)c4)c3)cc12]

5 -(2 -Chloropyridin-4 -yl)-1 H-indazol-3 -amine (SU1118 ) (0.070 g, 0.29 mmol), 3 -phenoxyaniline (0.25 g, 1.37 mmol), p-toluenesulfonic acid (0.009 g, 0.047 mmol) in 1,4 dioxane (2.5 mL) were heated in the microwave at 210° C. for 50 min. The reaction mixture was then cooled to room temperature and diluted with Et₂O (7 mL). The resulting solid was filtered under reduced pressure and washed with Et₂O (2×7 mL) and suspended in H₂O (5 mL) to which 10 % NH₃ (aqueous, 0.5 mL) was added. The resulting pH neutral solution was filtered under reduced pressure, washed with H₂O (30 mL) and Et₂O (20 mL) and dried. Further purification by HPLC (t_{R}=17 min) afforded the title compound as the TFA salt (0.0049 g, 0.013 mmol, 4.4 %). ¹ H NMR (DMSO-d₆ ): δ 6.60 (d, J=8.8 Hz, 1 H), 7.06 (m, 2 H), 7.15 (m, 3 H), 7.32 (t, J=8.0 Hz, 1 H), 7.42 (m, 5 H), 7.45 (br s, 1 H), 7.62 (dd, J=1.6, 8.8 Hz, 1 H), 8.16 (d, J=5.6 Hz, 1 H), 8.2 (s, 1 H), 11.81 (br s, 1 H), 11.84 (br s, 1 H). HRMS: For C₂₄H₂₀ON₅ requires 394.1660 found 394.1662 . (M+H)⁺

### 5 -(2 -((3 -(Benzyloxy)phenyl)amino)pyridin-4 -yl)-1 H-indazol-3 -amine SU1211

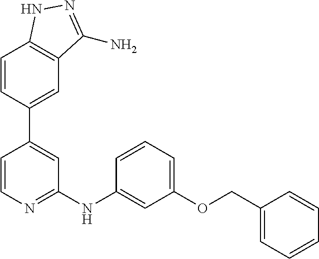 [SMILES: Nc1n[nH]c2ccc(-c3ccnc(Nc4cccc(OCc5ccccc5)c4)c3)cc12]

5 -(2 -Chloropyridin-4 -yl)-1 H-indazol-3 -amine (SU1118 ) (0.043 g, 0.18 mmol), 3 -(benzyloxy)aniline (175 μL, 0.88 mmol), p-toluenesulfonic acid (0.004 g, 0.02 mmol) in 1,4 dioxane (0.8 mL) were heated in the microwave at 210° C. for 50 min. The reaction mixture was then cooled to room temperature and diluted with Et₂O (7 mL). The resulting solid was filtered under reduced pressure and washed with Et₂O (2×7 mL) and suspended in H₂O (5 mL) to which 10 % NH₃ (aqueous, 0.5 mL) was added. The resulting pH neutral solution was filtered under reduced pressure, washed with H₂O and dried. Purification by column chromatography (100 % Hexane—50/50 Hexane/EtOAc—100 % EtOAc—10 % MeOH/EtOAc) and subsequent recrystalisation from EtOAc and Hexane afforded the desired compound (0.008 g, 0.02 mmol, 11.2 %) ¹H NMR (DMSO-d₆ ): δ 5.09 (s, 2 H), 5.48 (br s, 2 H), 6.55 (dd, J=7.5, 1.0 Hz, 1 H), 7.05 (m, 2 H), 7.10 (s, 1 H), 7.17 (m, 2 H), 7.35 (m, 2 H), 7.40 (t, J=14.5 Hz, 2 H), 7.49 (m, 2 H), 7.59 (t, J=4.5 Hz, 1 H), 8.12 (s, 1 H), 8.20 (d, J=5.5 Hz, 1 H), 9.07 (s, 1 H), 11.55 (br s, 1 H). HRMS: For C₂₅H₂₂ON₅ requires 408.1815, found 408.1819 . (M+H)⁺

### 5 -(2 -((4 -Fluorophenyl)amino)pyridine-4 -yl)-1 H-indazol-3 -amine SU1208

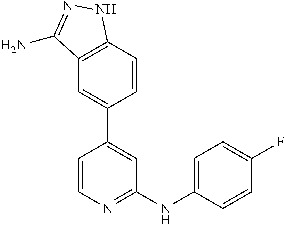 [SMILES: Nc1n[nH]c2ccc(-c3ccnc(Nc4ccc(F)cc4)c3)cc12]

A 0.5-2 mL microwave vial was charged with 5 -(2 -chloropyridin-4 -yl)-1 H-indazol-3 -amine (SU1118 ) (40 mg, 0.163 mmol, 1 eq.), 4 -fluoroaniline (362 mg, 312 μL, 0.326 mmol, 20 eq.), p-toluenesulfonic acid (3.1 mg, 0.163 mmol, 1 eq.) and 1,4 -dioxane (400 μL), was sealed with an aluminium crimp cap fitted with a disposable PTFE/silicon septum, and purged with nitrogen. The reaction mixture was heated to 210° C. under microwave irradiation for 50 minutes. The reaction mixture was then cooled down to ca. 40° C. and diluted by dropwise addition of Et₂O (7 mL) via syringe. The mixture was stirred and sonicated for 30 minutes and allowed to cool to room temperature. The cap was then removed and a solid was filtered, washed with Et₂O (2×7 mL), sonicated with a 2 % aqueous triethylamine (3×7 mL) and washed with H₂O (2×7 mL), the residue was suspended, sonicated and filtered using hot hexane (7 mL) and Et₂O (7 mL). The solid was dried to afford the title compound (30 mg, 58 %) as yellow solid. ¹H NMR (DMSO-D6 ) δ: 5.51 (s, 2 H), 7.07-7.02 (m, 2 H), 7.11 (t, J=8.8 Hz, 2 H), 7.34 (d, J=8.6 Hz, 1 H), 7.56 (dd, J=8.7, 1.5 Hz, 1 H), 7.76-7.68 (m, 2 H), 8.12 (s, 1 H), 8.17 (d, J=5.3 Hz, 1 H), 9.11 (s, 1 H), 11.56 (s, 1 H).

### 5 -(2 -((4 -Chlorophenyl)amino)pyridine-4 -yl)-1 H-indazol-3 -amine SU1209

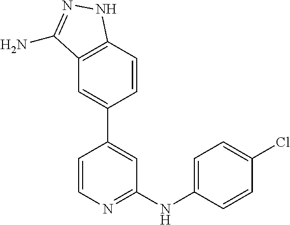 [SMILES: Nc1n[nH]c2ccc(-c3ccnc(Nc4ccc(Cl)cc4)c3)cc12]

A 0.5-2 mL microwave vial was charged with 5 -(2 -chloropyridin-4 -yl)-1 H-indazol-3 -amine (SU1118 ) (40 mg, 0.163 mmol, 1 eq.), 4 -chloroaniline (624 mg, 4.89 mmol, 30 eq.), p-toluenesulfonic acid (3.1 mg, 0.163 mmol, 1 eq.) and 1,4 -dioxane (600 μL), was sealed with an aluminium crimp cap fitted with a disposable PTFE/silicon septum and purged with nitrogen. The reaction mixture was heated to 210° C. under microwave irradiation for 50 minutes. The reaction mixture was then cooled down to ca. 40° C. and diluted by dropwise addition of Et₂O (7 mL) via syringe. The mixture was stirred and sonicated for 30 minutes and allowed to cool to room temperature. The cap was then removed and a solid was filtered, washed with Et₂O (2×7 mL), sonicated with a 2 % aqueous triethylamine (3×7 mL) and washed with H₂O (2×7 mL), the residue was suspended, sonicated and filtered using hot hexane (7 mL) and Et₂O (7 mL). The solid was dried to afford the title compound (25 mg, 45 %) as a goldenrod solid. ¹ H NMR (DMSO-D6 ) δ: 5.52 (s, 2 H), 7.12-7.03 (m, 2 H), 7.40-7.25 (m, 3 H), 7.57 (d, J=8.6 Hz, 1 H), 7.77 (d, J=8.8 Hz, 2 H), 8.13 (s, 1 H), 8.20 (d, J=5.3 Hz, 1 H), 9.26 (s, 1 H), 11.57 (s, 1 H).

### 4 -((4 -(3 -Amino-1 H-indazol-5 -yl)pyridin-2 -yl)amino)phenol SU1141

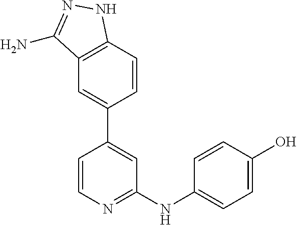 [SMILES: Nc1n[nH]c2ccc(-c3ccnc(Nc4ccc(O)cc4)c3)cc12]

A 0.5-2 mL microwave vial was charged with 5 -(2 -chloropyridin-4 -yl)-1 H-indazol-3 -amine (SU1118 ) (0.040 g, 0.16 mmol), 4 -aminophenol (0.178 g, 1.63 mmol), p-toluenesulfonic acid (0.003 g, 0.016 mmol) and 1,4 -dioxane (0.8 mL) was sealed with an aluminium crimp cap fitted with a disposable PTFE/silicon septum and purged with nitrogen. The reaction mixture was heated to 210° C. under microwave irradiation for 50 min. The reaction mixture was then cooled to 40° C. and diluted by dropwise addition of Et₂O (7 mL) via syringe. The mixture was stirred for 30 min and allowed to cool to room temperature. The cap was then removed and a solid was filtered, washed with Et₂O (2×7 mL) and suspended in H₂O (5 mL). To this suspension was added 10 % aqueous NH₃ (0.5 mL) and the mixture was heated to 35° C. at 800 mbar until the pH was neutral. The suspension was then filtered and washed with H₂O (3×7 mL). The solid was dried to afford the title compound as a black solid (0.026 g, 0.082 mmol, 50 %). ¹ H NMR (400 MHz, DMSO-d₆ ): δ 5.51 (s, 2 H), 6.88 (t, J=7.4 Hz, 1 H), 7.04 (dd, J=5.5, 1.5 Hz, , 1 H), 7.10 (s, 1 H), 7.27 (t, J=7.4 Hz, 2 H), 7.34 (d, J=8.6 Hz, 1 H), 7.57 (d, J=8.7 Hz, 1 H), 7.71 (d, J=7.6 Hz, 2 H), 8.12 (s, 1 H), 8.19 (d, J=5.4 Hz, 1 H), 9.08 (s, 1 H), 11.56 (br s, 1 H).

HRMS: Calculated for C₁₈H₁₆N₅O requires 318.13494 found 318.13513 (M+H)⁺.

### 5 -(2 -((4 -Methoxyphenyl)amino)pyridin-4 -yl)-1 H-indazol-3 -amine SU1120

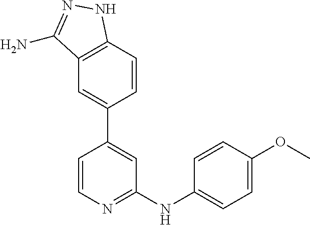 [SMILES: COc1ccc(Nc2cc(-c3ccc4[nH]nc(N)c4c3)ccn2)cc1]

A 0.5-2 mL microwave vial was charged with 5 -(2 -chloropyridin-4 -yl)-1 H-indazol-3 -amine (SU1118 ) (0.032 g, 0.131 mmol), 4 -methoxyaniline (0.048 g, 0.392 mmol) and 1,4 -dioxane (0.5 mL). This was then sealed with an aluminium crimp cap fitted with a disposable PTFE/silicon septum and purged with nitrogen. The reaction mixture was heated under microwave irradiation at 180° C. for 25 min and then at 190° C. for 50 min, at which point additional 4 -methoxyaniline (0.048 g, 0.392 mmol) and 1,4 -dioxane (0.5 mL) were added. The resulting mixture was heated to 210° C. under microwave irradiation for 50 min. The reaction mixture was then cooled to 40° C. and diluted by dropwise addition of Et₂O (7 mL) via syringe. The mixture was stirred for 30 min and allowed to cool to room temperature. The cap was then removed and a solid was filtered, washed with Et₂O (2×7 mL), triturated with 2 % aqueous triethylamine (3×7 mL), washed with H₂O (2×7 mL) and Et₂O (7 mL). The solid was dried to afford the title compound as a brown solid (0.012 g, 0.0362 mmol, 30 %). ¹H NMR (400 MHz, DMSO-d₆ ): δ 3.72 (s, 3 H), 5.50 (br s, 2 H), 6.88 (d, J=9.0 Hz, 2 H), 6.95-7.01 (m, 2 H), 7.33 (d, J=8.6 Hz, 1 H), 7.56 (t, J=10.2 Hz, 3 H), 8.07-8.14 (m, 2 H), 8.84 (s, 1 H), 11.55 (br s, 1 H).

HRMS: Calculated for C₁₉H₁₈N₅O requires 332.15059 found 332.15023 (M+H)⁺.

### 5 -(2 -{[4 -(Trifluoromethoxy)phenyl]amino}pyridine-4 -yl)-1 H-indazol-3 -amine SU1202

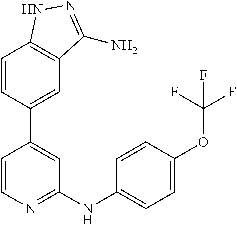 [SMILES: Nc1n[nH]c2ccc(-c3ccnc(Nc4ccc(OC(F)(F)F)cc4)c3)cc12]

5 -(2 -Chloropyridin-4 -yl)-1 H-indazol-3 -amine (SU1118 ) (0.045 g, 0.18 mmol), 4 -trifluoromethoxylaniline (120 μL, 0.89 mmol), p-toluenesulfonic acid (0.004 g, 0.020 mmol) in 1,4 dioxane (0.8 mL) were heated in the microwave at 210° C. for 50 min. The reaction mixture was then cooled to room temperature and diluted with Et₂O (7 mL). The resulting solid was filtered under reduced pressure and washed with Et₂O (2×7 mL) and suspended in H₂O (5 mL) to which 10 % NH₃ (aqueous, 0.5 mL) was added. The resulting pH neutral solution was filtered under reduced pressure, washed with H₂O and dried. Further purification by HPLC (t_{R}=18 min) afforded the title compound as the TFA salt (0.0054 g, 0.014 mmol, 7.6 %) ¹ H NMR (DMSO-d₆): 6 7.15 (d, J=5.6 Hz, 2 H), 7.32 (d, J=8.4 Hz, 2 H), 7.41 (d, J=8.8 Hz, 1 H), 7.67 (dd, J=8.8, 1.6 Hz, 1 H), 7.80 (d, J=8.8 Hz, 2 H), 8.21 (d, J=7.6 Hz, 2 H), 9.52 (br s, 1 H), 11.75 (br s, 1 H). HRMS: For C₁₉H₁₅0N₅ F₃ requires 386.1225, found 386.1223 . (M+H)⁺

### 5 -{2 -[4 -Propoxyphenyl)amino]pyridine-4 -y1 }-1 H-indazol-3 -amine SU1201

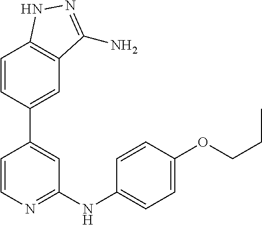 [SMILES: CCCOc1ccc(Nc2cc(-c3ccc4[nH]nc(N)c4c3)ccn2)cc1]

5 -(2 -Chloropyridin-4 -yl)-1 H-indazol-3 -amine (SU1118 ) (0.050 g, 0.20 mmol), 4 -isopropoxylaniline (150 μL, 1.00 mmol), p-toluenesulfonic acid (0.004 g, 0.02 mmol) in 1,4 dioxane (0.8 mL) were heated in the microwave at 210° C. for 50 min. The reaction mixture was then cooled to room temperature and diluted with Et₂O (7 mL). The resulting solid was filtered under reduced pressure and washed with Et₂O (2×7 mL) and suspended in H₂O (5 mL) to which 10 % NH₃ (aqueous, 0.5 mL) was added. The resulting pH neutral solution was filtered under reduced pressure, washed with H₂O and dried. Recrystallisation from dichloromethane and hexane afforded title compound as an off white solid (0.0058 g, 0.016 mmol, 7.4 %) ¹ H NMR (DMSO-d₆ ): δ 1.00 (t, J=7.4 Hz, 3 H), 1.76 (q, J=6.9 Hz, 2 H), 3.97 (t, J=6.4 Hz, 2 H), 7.06 (d, J=8.8 Hz, 2 H), 7.21 (s, 1 H), 7.25 (d, J=6.0 Hz, 1 H), 7.40 (m, 2 H), 7.65 (d, J=9.2 Hz, 1 H), 7.99 (d, J=6.8 Hz, 1 H), 8.31 (s, 1 H), 10.29 (br s, 1 H), 11.98 (br s, 1 H). HRMS: For C₂₁H₂₂ON₅ requires 360.1811, found 360.1819 . (M+H)+

### 2 -((4 -(3 -Amino-1 H-indazol-5 -yl)pyridin-2 -yl)amino)phenol SU1152

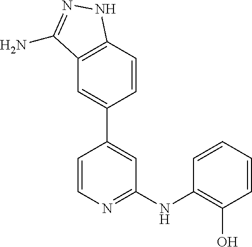 [SMILES: Nc1n[nH]c2ccc(-c3ccnc(Nc4ccccc4O)c3)cc12]

A 0.5-2 mL microwave vial was charged with 5 -(2 -chloropyridin-4 -yl)-1 H-indazol-3 -amine (SU1118 ) (0.037 g, 0.15 mmol), 2 -aminophenol (0.082 g, 0.76 mmol), p-toluenesulfonic acid (0.003 g, 0.02 mmol) and 1,4 -dioxane (0.5 mL) was sealed with an aluminium crimp cap fitted with a disposable PTFE/silicon septum and purged with nitrogen. The reaction mixture was heated to 210° C. under microwave irradiation for 50 min. The reaction mixture was then cooled to 40° C. and diluted by dropwise addition of Et₂O (7 mL) via syringe. The mixture was stirred for 30 min and allowed to cool to room temperature. The cap was then removed and a solid was filtered, washed with Et₂O (2×7 mL), and suspended in H₂O (5 mL). To this suspension was added 10 % aqueous NH₃ (0.5 mL) and the mixture was heated to 35° C. at 800 mbar until the pH was neutral. The suspension was then filtered and washed with H₂O (3×7 mL). The solid was dried to afford the title compound as a dark orange solid (0.018 g, 0.057 mmol, 38 %). ¹ H NMR (400 MHz, DMSO-d₆): 6 5.50 (s, 2 H), 6.27-6.33 (m, 1 H), 6.98-7.13 (m, 4 H), 7.27 -7.37 (m, 2 H), 7.56 (d, J=8.7 Hz, 1 H), 8.11 (s, 1 H), 8.18 (d, J=5.4 Hz, 1 H), 8.95 (s, 1 H), 9.19 (br s, 1 H), 11.56 (br s, 1 H). HRMS: Calculated for C₁₈H₁₆N₅O requires 318.13494 found 318.13483 (M+H)⁺.

### 4 -{[4 -(3 -Amino-1 H-indazol-5 -yl)pyridin-2 -yl]amino)-2 -methylphenol SU1191

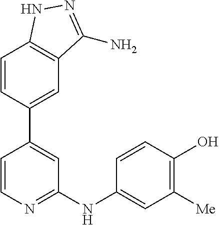 [SMILES: Cc1cc(Nc2cc(-c3ccc4[nH]nc(N)c4c3)ccn2)ccc1O]

5 -(2 -Chloropyridin-4 -yl)-1 H-indazol-3 -amine (SU1118 ) (0.035 g, 0.14 mmol), 4 -amino-o-cresol (0.186 g, 1.43 mmol), p-toluenesulfonic acid (0.003 g, 0.02 mmol) in 1,4 dioxane (0.8 mL) were heated in the microwave at 210° C. for 50 min. The reaction mixture was then cooled to room temperature and diluted with Et₂O (7 mL). The resulting solid was filtered under reduced pressure and washed with Et₂O (2×7 mL) and suspended in H₂O (5 mL) to which 10 % NH₃ (aqueous, 0.5 mL) was added. The resulting pH neutral solution was filtered under reduced pressure, washed with H₂O and dried to afford the title compound as yellow solid (0.0033 g, 0.001 mmol, 6.9 %). ¹H NMR (DMSO-d₆): 8 2.12 (s, 3 H), 5.48 (br s, 2 H), 6.71 (d, J=8.4 Hz, 1 H), 6.94 (m, 2 H), 7.25 (d, J=9.6 Hz, 2 H), 7.32 (d, J=8.8 Hz, 1 H), 7.52 (d, J=8.0 Hz, 1 H), 8.09 (d, J=6.0 Hz, 2 H), 8.60 (br s, 1 H), 8.84 (s, 1 H), 11.53 (br s, 1 H,). HRMS: For C₁₉H₁₈ON₅ requires 332.1503, found 332.1506 . (M+H)⁺

### 5 -{[-4 -(3 -Amino-1 H-indazol-5 -yl)pyridin-2 -yl]amino}-2 -methylphenol SU1195

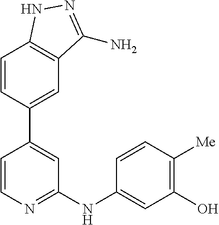 [SMILES: Cc1ccc(Nc2cc(-c3ccc4[nH]nc(N)c4c3)ccn2)cc1O]

5 -(2 -Chloropyridin-4 -yl)-1 H-indazol-3 -amine (SU1118 ) (0.045 g, 0.18 mmol), 5 -amino-o-cresol (0.12 g, 0.96 mmol), p-toluenesulfonic acid (0.003 g, 0.015 mmol) in 1,4 dioxane (0.8 mL) were heated in the microwave at 210° C. for 50 min. The reaction mixture was then cooled to room temperature and diluted with Et₂O (7 mL). The resulting solid was filtered under reduced pressure and washed with Et₂O (2×7 mL) and suspended in H₂O (5 mL) to which 10 % NH₃ (aqueous, 0.5 mL) was added. The resulting pH neutral solution was filtered under reduced pressure, washed with H₂O and dried. Further purification by HPLC (t_{R}=14 min) afforded the title compound as the TFA salt (0.0041 g, 0.012 mmol, 6.7 %) (LC-MS purity >99 %). ¹ H NMR (DMSO-d₆ ): δ 2.11 (s, 3 H), 6.85 (d, J=6.0 Hz, 1 H), 7.00 (m, 1 H), 7.08 (m, 1 H), 7.20 (m, 2 H), 7.38 (d, J=8.5 Hz, 1 H), 7.63 (d, J=8.5 Hz, 1 H), 8.06 (d, J=5.0 Hz, 1 H), 8.24 (s, 1 H), 9.49 (br s, 1 H), 11.79 (br s, 1 H). HRMS: For C₁H₁₆0N₅ requires 330.1365, found 330.1368 . (M+H)⁺

### 4 -{[4 -(3 -Amino-1 H-indazol-5 -yl)pyridin-2 -yl]amino}-3 -methylphenol SU1192

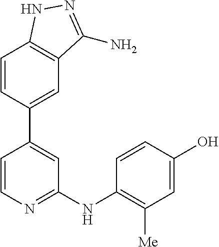 [SMILES: Cc1cc(O)ccc1Nc1cc(-c2ccc3[nH]nc(N)c3c2)ccn1]

5 -(2 -Chloropyridin-4 -yl)-1 H-indazol-3 -amine (SU1118 ) (0.037 g, 0.15 mmol), 4 -amino-m-cresol (0.093 g, 0.75 mmol), p-toluenesulfonic acid (0.003 g, 0.02 mmol) in 1,4 dioxane (0.8 mL) were heated in the microwave at 210° C. for 50 min. The reaction mixture was then cooled to room temperature and diluted with Et₂O (7 mL). The resulting solid was filtered under reduced pressure and washed with Et₂O (2×7 mL) and suspended in H₂O (5 mL) to which 10 % NH₃ (aqueous, 0.5 mL) was added. The resulting pH neutral solution was filtered under reduced pressure, washed with H₂O and dried. Further purification by HPLC (t_{R}=10 min) afforded the title compound as the TFA salt (0.005 g, 0.014 mmol, 9.2 %). ¹H NMR (DMSO-d₆ ): δ 2.15 (s, 3 H), 6.75 (dd, J=8.5, 2.5 Hz, 1 H), 6.82 (d, J=2.5 Hz, 1 H), 7.19 (d, J=5.5 Hz, 2 H), 7.26 (d, J=6.5 Hz, 1 H), 7.40 (d, J=9.0 Hz, 1 H,), 6.22 (dd, J=10.0, 8.5 Hz, 1 H,), 7.89 (d, J=7.0 Hz, 1 H), 8.31 (s, 1 H), 9.71 (br s, 1 H), 9.96 (br s, 1 H), 11.86 (br s, 1 H). HRMS: For C₁₉H₁₈ON₅ requires 332.1506, found 332.1506 . (M+H)⁺5 -{2 -[(3,4 -Dichlorophenyl)amino]pyridin-4 -yl}-1 H-indazol-3 -amine SU1223

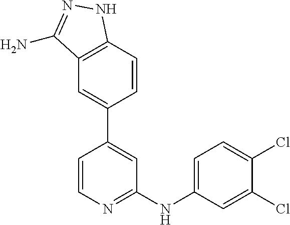 [SMILES: Nc1n[nH]c2ccc(-c3ccnc(Nc4ccc(Cl)c(Cl)c4)c3)cc12]

5 -(2 -Chloropyridin-4 -yl)-1 H-indazol-3 -amine (SU1118 ) (0.046 g, 0.19 mmol), 3,4 -dichloroaniline (0.161 g, 1.00 mmol), p-toluenesulfonic acid (0.004 g, 0.02 mmol) in 1,4 dioxane (0.8 mL) were heated in the microwave at 210° C. for 50 min. The reaction mixture was then cooled to room temperature and diluted with Et₂O (7 mL). The resulting solid was filtered under reduced pressure and washed with Et₂O (2×7 mL) and suspended in H₂O (5 mL) to which 10 % NH₃(aqueous, 0.5 mL) was added. The resulting pH neutral solution was filtered under reduced pressure, washed with H₂O and dried. Further purification by HPLC (t_{R}=18 min) afforded the title compound as the TFA salt (0.0022 g, 0.01 mmol, 3.2 %). ¹H NMR (DMSO-d₆ ): δ 7.12 (s, 1 H), 7.15 (dd, J=5.6, 1.6 Hz, 1 H), 7.37 (d, J=8.8 Hz, 1 H), 7.49 (d, J=8.8 Hz, 1 H), 7.60 (m, 2 H), 8.16 (s, 1 H,), 8.26 (m, 2 H,), 9.48 (s, 1 H,), 11.66 (br s, 1 H).

### Route to SU1256

### N-(4 -chloropyridin-2 -yl)thiazol-2 -amine

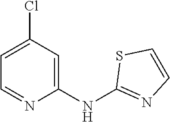 [SMILES: Clc1ccnc(Nc2nccs2)c1]

A 2-5 mL microwave vial was charged with palladium acetate (0.0067 g, 0.03 mmol, 2 mol %) and xantphos (0.026 g, 0.045 mmol, 3 mol %), sealed with an aluminium crimp cap fitted with a disposable PTFE/silicon septum, and purged with nitrogen before adding toluene (5 mL, 02 -free). The resulting solution was stirred under nitrogen for 10 minutes and then transferred to a sealed 10-20 mL microwave vial containing a nitrogen-purged mixture of 2 -bromo-4 -chloropyridine (0.289 g, ca. 166 μL, 1.5 mmol, 1 eq.), thiazol-2 -amine (0.18 g, 1.8 mmol, 1.2 eq.), and anhydrous cesium carbonate (0.684 g, 2.1 mmol, 1.4 eq.) in toluene (5 mL). The resulting mixture was purged with N₂ at ca. 100° C. for 5 minutes and then heated to 110° C. for 18 hours. The reaction mixture was then cooled to room temperature, diluted with dichloromethane (10 mL) and filtered; the precipitate was washed with dichloromethane (20 mL×3 ). The combined organic filtrates were concentrated onto silica gel and purified by flash chromatography (Biotage SP4, 50 g SiO₄, 20 % EtOAc in hexane) to afford the title compound as a white solid (0.102 g, 32 %). 1 H NMR (400 MHz, DMSO-d6 ) δ 7.02 (dd, J=5.5, 1.8 Hz, 1 H), 7.06 (d, J=3.5 Hz, 1 H), 7.14 (s, 1 H), 7.41 (d, J=3.6 Hz, 1 H), 8.28 (d, J=5.6 Hz, 1 H), 11.41 (s, 1 H).

### N-(4 -(3 -Amino-1 H-indazol-5 -yl)pyridin-2 -yl)thiazol-2 -amine SU1256

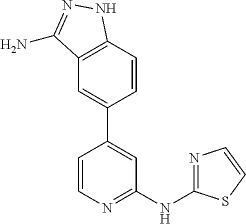 [SMILES: Nc1n[nH]c2ccc(-c3ccnc(Nc4nccs4)c3)cc12]

A 2-5 mL microwave vial was charged with N-(4 -chloropyridin-2 -yl)thiazol-2 -amine (0.074 g, 0.350 mmol, 1 eq.), 5 -(4,4,5,5 -tetramethyl-1,3,2 -dioxaborolan-2 -yl)-1 H-indazol-3 -amine (0.127 g, 0.578 mmol, 1.4 eq.), [1,1 ′-bis(di-tert-butylphosphino)ferrocene]dichloropalladium(II) catalyst (0.011 g, 0.0175 mmol, 5 mol %), sealed with an aluminium crimp cap fitted with a disposable PTFE/silicon septum, and purged with nitrogen. Ethanol (1.4 mL) was added, and the mixture was stirred under nitrogen for 10 minutes before adding K₃ PO₄ (0.7 mL, 1 M in water, 02 -free). The reaction mixture was heated to ca. 50° C., purged with nitrogen for 10 minutes and heated to 110° C. for 28 hours. The reaction mixture was then stirred at room temperature, diluted by dropwise addition of water (ca. 8 mL) and filtered. The filtered solid was washed with water (7 mL×2 ) and hexane (7 mL×2 ) and purified by flash chromatography (Biotage SP4, 50 g SiO₄, 5 % MeOH in EtOAc) to afford the title compound as a light yellow solid (0.027 g, 0.0876 mmol, 25 %). 1 H NMR (400 MHz, DMSO-d6 ) δ 5.53 (s, 2 H), 7.01 (d, J=3.5 Hz, 1 H), 7.20 (d, J=5.5 Hz, 1 H), 7.32 (s, 1 H), 7.36 (d, J=8.7 Hz, 1 H), 7.39 (d, J=3.5 Hz, 1 H), 7.56 (d, J=8.7 Hz, 1 H), 8.14 (s, 1 H), 8.32 (d, J=5.4 Hz, 1 H), 11.28 (s, 1 H), 11.59 (s, 1 H). HRMS: Calculated for C₁₅H₁₃N₆ S (M+H⁺)=309.0917 ; Found: 309.0914 (M+H)⁺

### 5 -(2 -(Pyrimidin-2 -ylamino)pyridin-4 -yl)-1 H-indazol-3 -amine SU1245

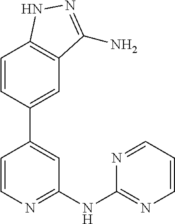 [SMILES: Nc1n[nH]c2ccc(-c3ccnc(Nc4ncccn4)c3)cc12]

To N-(4 -chloropyridin-2 -yl)pyrimidin-2 -amine (0.045 g, 0.22 mmol), 5 -(4,4,5,5 -tetramethyl-1,3,2 -dioxaborolan-2 -yl)-1 H-indazol-3 -amine (0.098 g, 0.38 mmol) and [1,1 ′-bis(di-tert-butylphosphino)ferrocene]dichloropalladium(II) catalyst (0.018 g, 0.028 mmol) in EtOH (1.6 mL, degassed under nitrogen) was added K₃ PO₄ (1 M, 0.8 mL) and the reaction mixture was heated to 100° C. for 20 h. The reaction mixture was cooled to room temperature and water (7 mL) was added and the reaction was stirred at room temperature for 5 mins. The reaction mixture was filtered and the resulting solid was washed with water (10 mL), hexane (20 mL), EtOAc (10 mL) and MeOH (8 mL) and then dried to afford the title compound as an orange solid (0.021 g, 0.07 mmol, 32 %). ¹H NMR (DMSO-d₆ ): δ 5.51 (br s, 2 H), 6.98 (t, J=4.8 Hz, 1 H), 7.28 (dd, J=1.6, 5.6 Hz, 1 H), 7.36 (d, J=8.8 Hz, 1 H), 7.63 (dd, J=1.2, 8.4 Hz, 1 H), 8.17 (s, 1 H), 8.31 (d, J=5.2 Hz, 1 H), 8.57 (s, 1 H), 8.59 (d, J=4.8 Hz, 2 H), 9.76 (s, 1 H), 11.56 (br s, 1 H). HRMS: For C₁₆H₁₄N₇ requires 304.1305 found 304.1300.

### 5 -(2 -(Pyridin-4 -ylamino)pyridin-4 -yl)-1 H-indazol-3 -amine SU1233

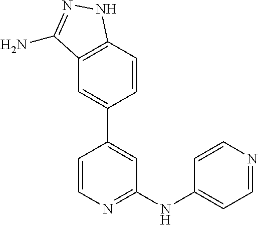 [SMILES: Nc1n[nH]c2ccc(-c3ccnc(Nc4ccncc4)c3)cc12]

A 2-5 mL microwave vial was charged with 4 -chloro-N-(pyridin-4 -yl)pyridin-2 -amine (SU1118 ) (0.085 g, 0.41 mmol, 1 eq), 5 -(4,4,5,5 -tetramethyl-1,3,2 -dioxaborolan-2 -yl)-1 H-indazol-3 -amine (0.150 g, 0.58 mmol, 1.4 eq.) and [1,1 ′-bis(di-tert-butylphosphino)ferrocene]dichloropalladium(II) catalyst, (0.013 g, 0.02 mmol, 5 mol %), sealed with an aluminium crimp cap fitted with a disposable PTFE/silicon septum, and purged with nitrogen. Ethanol (1.6 mL) was added, and the mixture was stirred under nitrogen for some minutes before adding K₃ PO₄ (0.8 mL, 1 M in water, O₂ -free). The mixture was heated to ca. 50° C. and purged with nitrogen for 10 minutes. The resulting mixture was then left stirring at 100° C. for 24 hours, during which time the initial clear orange solution became cloudy. The reaction mixture was then stirred at room temperature and diluted by dropwise addition of water (ca. 8 mL) to give a suspension that was stirred for some hours before being filtered. The filtered solid was washed with water (7 mL×2 ) and hexane (7 mL×2 ). The resulting solid was then resuspended in EtOAc/MeOH (7 mL×4 ), filtered and evaporated to yield the title compound. (0.113 g, 0.37 mmol, 90 %) ¹H NMR (DMSO-D6 ) 6: 5.52 (s, 2 H), 7.24-7.17 (m, 2 H), 7.36 (d, J=8.8 Hz, 1 H), 7.60 (dd, J=8.7, 1.6 Hz, 1 H), 7.71-7.68 (m, 2 H), 8.16 (s, 1 H), 8.37-8.24 (m, 3 H), 9.61 (s, 1 H), 11.59 (s, 1 H), HRMS: Calculated for C1₇ H,₅N₆ (M+H⁺): 303.1353 ; Found: 303.1349

### 5 -(2 -(Pyridin-3 -ylamino)pyridin-4 -yl)-1 H-indazol-3 -amine SU1234

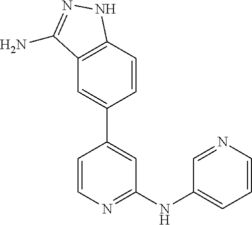 [SMILES: Nc1n[nH]c2ccc(-c3ccnc(Nc4cccnc4)c3)cc12]

A 0.5-2 mL microwave vial was charged with 4 -chloro-N-(pyridin-3 -yl)pyridin-2 -amine (SU1118 ) (0.060 mg, 0.29 mmol, 1 eq), 5 -(4,4,5,5 -tetramethyl-1,3,2 -dioxaborolan-2 -yl)-1 H-indazol-3 -amine (0.106 g, 0.41 mmol, 1.4 eq.), and [1,1 ′-bis(di-tert-butylphosphino)ferrocene]dichloropalladium(II) catalyst (0.010 g, 0.015 mmol, 5 mol %), sealed with an aluminium crimp cap fitted with a disposable PTFE/silicon septum, and purged with nitrogen. Ethanol (1.2 mL) was added, and the mixture was stirred under nitrogen for some minutes before adding K₃ PO₄ (0.6 mL, 1 M in water, O₂ -free). The mixture was heated to ca. 50° C. and purged with nitrogen for 10 minutes. The resulting mixture was then left stirring at 100° C. for 24 hours, during which time the initial clear orange solution became cloudy. The reaction mixture was then stirred at room temperature and diluted by dropwise addition of water (ca. 6 mL) to give a suspension that was stirred for some hours before being filtered. The filtered solid was washed with water (7 mL×2 ) and hexane (7 mL×2 ). The resulting solid was then resuspended in EtOAc (7 mL×3 ), filtered, and evaporated to yield the title compound. (68 mg, 0.22 mmol, 77 %). ¹H NMR (DMSO-D6 ) 6: 5.51 (s, 2 H), 7.17-7.08 (m, 2 H), 7.32-7.25 (m, 1 H), 7.35 (d, J=8.8 Hz, 1 H), 7.58 (d, J=8.6 Hz, 1 H), 8.09 (d, J=4.1 Hz, 1 H), 8.14 (s, 1 H), 8.22 (d, J=5.3 Hz, 1 H), 8.26 (d, J=8.1 Hz, 1 H), 8.82 (s, 1 H), 9.31 (s, 1 H), 11.58 (s, 1 H), HRMS: Calculated for C₁₇H₁₅N₆ (M+H*): 303.1353 ; Found: 303.1352 (M+H)⁺

### 5 -(2 -(Pyridin-2 -ylamino)pyridin-4 -yl)-1 H-indazol-3 -amine SU1213

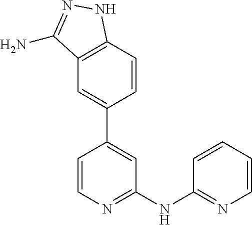 [SMILES: Nc1n[nH]c2ccc(-c3ccnc(Nc4ccccn4)c3)cc12]

A 2-5 mL microwave vial was charged with 4 -chloro-N-(pyridin-2 -yl)pyridin-2 -amine (SU1118 ) (0.085 g, 0.41 mmol, 1 eq), 5 -(4,4,5,5 -tetramethyl-1,3,2 -dioxaborolan-2 -yl)-1 H-indazol-3 -amine (0.150 g, 0.58 mmol, 1.4 eq.), and [1,1 ′-bis(di-tert-butylphosphino)ferrocene]dichloropalladium(II) catalyst (0.013 g, 0.02 mmol, 5 mol %), sealed with an aluminium crimp cap fitted with a disposable PTFE/silicon septum, and purged with nitrogen. Ethanol (1.6 mL) was added, and the mixture was stirred under nitrogen for some minutes before adding K₃ PO₄ (0.8 mL, 1 M in water, O₂ -free). The mixture was heated to ca. 50° C. and purged with nitrogen for 10 minutes. The resulting mixture was then left stirring at 100° C. for 24 hours, during which time the initial clear orange solution became cloudy. The reaction mixture was then stirred at room temperature and diluted by dropwise addition of water (ca. 8 mL) to give a suspension that was stirred for some hours before being filtered. The filtered solid was washed with water (7 mL×2 ) and hexane (7 mL×2 ). The resulting solid was then resuspended in EtOAc (7 mL×2 ), filtered and evaporated to yield the title compound. (0.110 g, 0.36 mmol, 88 %) ¹H NMR (DMSO-D6 ) 6: 5.52 (s, 2 H), 6.90-6.85 (m, 1 H), 7.16 (dd, J=5.3, 1.4 Hz, 1 H), 7.36 (d, J=8.7 Hz, 1 H), 7.58 (dd, J=8.7, 1.4 Hz, 1 H), 7.70-7.63 (m, 1 H), 7.79 (d, J=8.5 Hz, 1 H), 8.02 (s, 1 H), 8.14 (s, 1 H), 8.25 (d, J=5.2 Hz, 2 H), 9.68 (s, 1 H), 11.57 (s, 1 H).

### Route to SU1254

### N² -(4 -chloropyridin-2 -yl)pyridine-2,6 -diamine

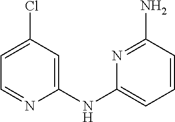 [SMILES: Nc1cccc(Nc2cc(Cl)ccn2)n1]

A 2-5 mL microwave vial was charged with palladium acetate (0.009 g, 0.04 mmol, 2 mol %) and xantphos (0.035 g, 0.06 mmol, 3 mol %), sealed with an aluminium crimp cap fitted with a disposable PTFE/silicon septum, and purged with nitrogen before adding toluene (5 mL, O₂ -free). The resulting solution was stirred under nitrogen for 10 minutes and then transferred to a sealed 10-20 mL microwave vial containing a nitrogen-purged mixture of 2 -bromo-4 -chloropyridine (0.385 g, ca. 221 μL, 2 mmol, 1 eq.), pyridine-2,6 -diamine (0.38 g, 2 mmol, 1 eq.), and anhydrous cesium carbonate (0.912 g, 2.8 mmol, 1.4 eq.) in toluene (5 mL). The resulting mixture was heated to ca. 100° C. under nitrogen for 5 minutes and then left stirring at 100° C. for 17 hours. The reaction mixture was then cooled to room temperature and filtered, the precipitate was washed with dichloromethane (10 mL×3 ) and EtOAc (7 mL). The combined organic filtrates were concentrated onto silica gel and purified by flash chromatography (Biotage SP4, 100 g SiO₄, 31 % EtOAc in hexane) to afford the title compound as a white solid (0.177 g, 0.530 mmol, 40 %). ¹H NMR (400 MHz, DMSO-d₆ ) δ 5.79 (s, 2 H), 5.99 (d, J=7.9 Hz, 1 H), 6.68 (d, J=7.8 Hz, 1 H), 6.88 (dd, J=5.4, 1.8 Hz, 1 H), 7.26 (t, J=7.9 Hz, 1 H), 8.03 (d, J=1.6 Hz, 1 H), 8.13 (d, J=5.4 Hz, 1 H), 9.42 (s, 1 H).

### N² -(4 -(3 -Amino-1 H-indazol-5 -ylpyridine-2 -yl)pyridine-2,6 -diamine SU1254

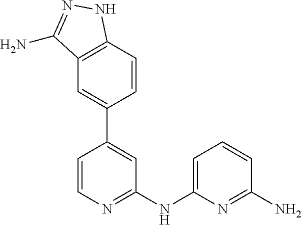 [SMILES: Nc1cccc(Nc2cc(-c3ccc4[nH]nc(N)c4c3)ccn2)n1]

A 2-5 mL microwave vial was charged with N² -(4 -chloropyridin-2 -yl)pyridine-2,6 -diamine (0.091 g, 0.413 mmol, 1 eq), 5 -(4,4,5,5 -tetramethyl-1,3,2 -dioxaborolan-2 -yl)-1 H-indazol-3 -amine (0.150 g, 0.578 mmol, 1.4 eq.), [1,1 ′-bis(di-tert-butylphosphino)ferrocene]dichloropalladium(II) catalyst (0.013 g, 0.0207 mmol, 5 mol %), sealed with an aluminium crimp cap fitted with a disposable PTFE/silicon septum, and purged with nitrogen. Ethanol (1.6 mL) was added, and the mixture was stirred under nitrogen for 10 minutes before adding K₃ PO₄ (0.8 mL, 1 M in water, O₂ -free). The mixture was heated to ca. 50° C. and purged with nitrogen for 10 minutes. The resulting mixture was then heated to 100° C. for 24 hours, during which time the initial clear orange solution became cloudy. The reaction mixture was then stirred at room temperature and diluted by dropwise addition of water (ca. 8 mL) to give a suspension that was stirred for 30 minutes before being filtered. The filtered solid was washed with water (7 mL×2 ) and hexane (7 mL×2 ). The resulting solid was then dissolved in 20 % MeOH in EtOAc (7 mL×2 ), filtered and evaporated to yield the title compound as a beige solid (0.110 g, 0.347 mmol, 84 %). 1 H NMR (400 MHz, DMSO-d6 ) δ 5.52 (s, 2 H), 5.73 (s, 2 H), 5.98 (d, J=7.8 Hz, 1 H), 6.81 (d, J=7.9 Hz, 1 H), 7.10 (d, J=5.3 Hz, 1 H), 7.27 (t, J=7.9 Hz, 1 H), 7.34 (d, J=8.6 Hz, 1 H), 7.63 (d, J=8.8 Hz, 1 H), 8.12 (s, 1 H), 8.17 (s, 1 H), 8.19 (d, J=5.4 Hz, 1 H), 9.13 (s, 1 H), 11.54 (s, 1 H).HRMS: Calculated for C₁₇H₁₆N₇ (M+H⁺)=318.1462 ; Found: 318.1464

### Route to SU1262

### N-Benzyl-6 -bromopyridin-2 -amine

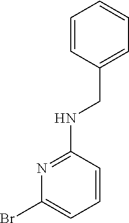 [SMILES: Brc1cccc(NCc2ccccc2)n1]

A 0.5-2 mL microwave vial was charged with 2 -bromo-6 -fluoropyridine (0.352 g, 2 mmol, 1 eq.) phenylmethanamine (0.643 g, 655 μL, 6 mmol, 3 eq.) and dioxane (0.7 mL, anhydrous), sealed with an aluminium crimp cap fitted with a disposable PTFE/silicon septum and purged with nitrogen. The resulting solution was heated to 180° C. under microwave irradiation for 50minutes. The reaction mixture was then cooled to room temperature, diluted with dropwise addition of water (7 mL), sonicated and filtered. The resulting precipitate was washed with water (4 mL×3 ) and hexane (2 mL×3 ), and dried to afford the title compound (0.489 g, 93 %) as white solid. 1 H NMR (400 MHz, DMSO-d6 ) δ 4.41 (d, J=6.0 Hz, 2 H), 6.47 (d, J=8.2 Hz, 1 H), 6.64 (d, J=7.4 Hz, 1 H), 7.16-7.40 (m, 6 H), 7.48 (t, J=5.8 Hz, 1 H).

### N² -Benzyl-N⁶ -(4 -chloropyridin-2 -yl)pyridine-2,6 -diamine

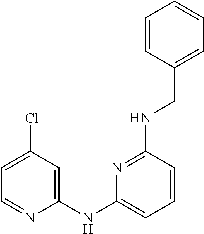 [SMILES: Clc1ccnc(Nc2cccc(NCc3ccccc3)n2)c1]

A 2-5 mL microwave vial was charged with palladium acetate (0.0072 g, 0.032 mmol, 2 mol %) and xantphos (0.028 g, 0.048 mmol, 3 mol %), sealed with an aluminium crimp cap fitted with a disposable PTFE/silicon septum, and purged with nitrogen before adding toluene (4 mL, 02 -free). The resulting solution was stirred under nitrogen for 10 minutes and then transferred to a sealed 10-20 mL microwave vial containing a nitrogen-purged mixture of 4 -chloropyridin-2 -amine (0.309 g, 2.4 mmol, 1.5 eq.), N-benzyl-6 -bromopyridin-2 -amine (0.421 g, 1.6 mmol, 1 eq.), and anhydrous cesium carbonate (1.04 g, 3.2 mmol, 2 eq.) in toluene (4 mL). The resulting mixture was purged with N₂ at ca. 100° C. for 5 minutes and then heated to 110° C. for 17 hours. The reaction mixture was then cooled to room temperature and filtered, the precipitate was washed with dichloromethane (10 mL×3 ) and EtOAc (7 mL). The combined organic filtrates were concentrated onto silica gel and purified by flash chromatography (Biotage SP4, 100 g SiO₄, 14 % MeOH in EtOAc) to afford the title compound as a beige solid (0.176 g, 35 %). 1 H NMR (400 MHz, DMSO-d6 ) δ 4.51 (d, J=6.1 Hz, 2 H), 6.06 (d, J=7.9 Hz, 1 H), 6.51 (d, J=7.7 Hz, 1 H), 6.87 (dd, J=5.4, 2.0 Hz, 1 H), 7.04 (t, J=6.0 Hz, 1 H), 7.20 (t, J=7.2 Hz, 1 H), 7.28 (dt, J=12.5, 7.6 Hz, 3 H), 7.36 (d, J=7.1 Hz, 2 H), 8.12 (d, J=5.4 Hz, 1 H), 8.19 (d, J=1.8 Hz, 1 H), 9.48 (s, 1 H).

### N² -(4 -(3 -Amino-1 H-indazol-5 -ylpyridine-2 -yl)-N-benzylpyridine-2,6 -diamine SU1262

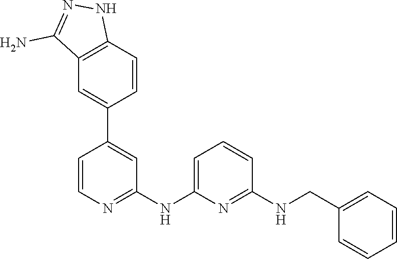 [SMILES: Nc1n[nH]c2ccc(-c3ccnc(Nc4cccc(NCc5ccccc5)n4)c3)cc12]

A 0.5-2 mL microwave vial was charged with N₂ -benzyl-N₆ -(4 -chloropyridin-2 -yl)pyridine-2,6 -diamine (0.086 g, 0.277 mmol, 1 eq.), 5 -(4,4,5,5 -tetramethyl-1,3,2 -dioxaborolan-2 -yl)-1 H-indazol-3 -amine (0.1 g, 0.387 mmol, 1.4 eq.), [1,1 ′-bis(di-tert-butylphosphino)ferrocene]dichloropalladium(II) catalyst (0.009 g, 0.0139 mmol, 5 mol %), sealed with an aluminium crimp cap fitted with a disposable PTFE/silicon septum, and purged with nitrogen. Ethanol (1.1 mL) was added, and the mixture was stirred under nitrogen for 10 minutes before adding K₃ PO₄ (0.55 mL, 1 M in water, 02 -free). The reaction mixture was heated to ca. 50° C., purged with nitrogen for 10 minutes and heated to 100° C. for 24 hours. The reaction mixture was then stirred at room temperature and diluted by dropwise addition of water (ca. 6 mL) to give a suspension that was stirred and filtered. The resulting solid was washed with water (7 mL×2 ) and hexane (7 mL×2 ), dissolved in 20 % MeOH in EtOAc (7 mL), filtered and evaporated to yield the title compound as a beige solid (0.104 g, 0.255 mmol, 92 %). LCMS 1 H NMR (400 MHz, DMSO-d6 ) δ 4.54 (d, J=5.7 Hz, 2 H), 5.50 (s, 2 H), 6.02 (d, J=7.9 Hz, 1 H), 6.67 (d, J=7.9 Hz, 1 H), 6.88 (t, J=5.8 Hz, 1 H), 7.08 (d, J=5.4 Hz, 1 H), 7.15 -7.30 (m, 7 H), 7.53 (d, J=8.8 Hz, 1 H), 8.15 (s, 1 H), 8.19 (d, J=5.3 Hz, 1 H), 8.33 (s, 1 H), 9.21 (s, 1 H), 11.52 (s, 1 H). HRMS: Calculated for C₂₄H₂₂N₇ (M+H*)=408.1931 ; Found: 408.1934

Azaindole Ring Incorporated into a Tricyclic System

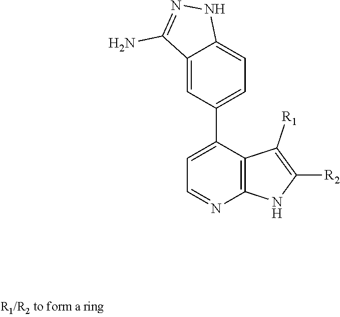

### 5 -(9 H-Pyrido[2,3 -b]indol-4 -yl)-1 H-indazol-3 -amine SU1087

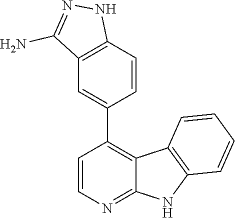 [SMILES: Nc1n[nH]c2ccc(-c3ccnc4[nH]c5ccccc5c34)cc12]

4 -Chloro-α-carboline (0.120 g, 0.59 mmol), 3 -cyano-4 -fluorophenyl boronic acid (0.176 g, 0.71 mmol), [1,1 ′-bis(di-tert-butylphosphino)ferrocene]dichloropalladium(II) catalyst (0.040 g, 0.06 mmol) and a magnetic stirrer bar were added in 2-5 mL MW vial which was capped and purged with N₂ for 10 min. To this was added a mixture of IPA:H₂O (4.5 mL, 2:1 ) and t-butylamine (310 μL, 2.95 mmol) and the vial was flushed again under N₂ for 10 min. The vial was subjected to MW irradiation for 45 min at 160° C. After cooling, the solvent was concentrated under reduced pressure. The suspension of crude 4 -(2 -fluoro-1 -cyanophenyl-5 -yl)-a-carboline in ethanol (4.5 mL) and a magnetic stirrer were added in 2-5 mL MW vial which was capped and purged with N₂ for 10 min. Hydrazine hydrate (100 μL, 2.95 mmol) was added and the vial was subjected to MW irradiation for 30 min at 165° C. After cooling, the reaction mixture was filtered and washed with ethanol (20 mL) and the filtrates were evaporated in vacuo. The solid residue was purified by flash column using a gradient from dichloromethane to dichlorormethane:MeOH 9:1 to afford the title compound as a white powder (0.061 g, 0.20 mmol, 35 %). ¹H NMR (400 MHz, DMSO-d6 ): δ 5.47 (br s, 2 H), 7.02-6.98 (m, 1 H), 7.11 (d, J=5.0 Hz, 1 H), 7.42-7.38 (m, 1 H), 7.44 (d, J=8.5 Hz, 1 H), 7.51 (d, J=8.1 Hz, 1 H), 7.54 (dd, J=8.7, 1.4 Hz, 1 H), 7.57 (d, J=8.1 Hz, 1 H), 8.05 (s, 1 H), 8.44 (d, J=5.0 Hz, 1 H), 11.62 (br s, 1 H), 11.90 (br s, 1 H). HRMS: For C₁₈H₁₃N₅ requires 299.12 found 300.3 (M+H)⁺.

### 5 -(8 -Methyl-9 H-pyrido[2,3 -b]indol-4 -yl)-1 H-indazol-3 -amine SU1170

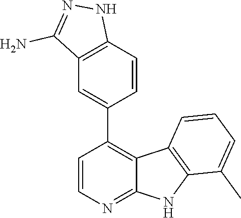 [SMILES: Cc1cccc2c1[nH]c1nccc(-c3ccc4[nH]nc(N)c4c3)c12]

4 -Chloro-8 -methyl-α-carboline (0.09 g, 0.42 mmol), 3 -cyano-4 -fluorophenylboronic acid (0.123 g, 0.50 mmol)41,1 ′-bis(di-tert-butylphosphino)ferrocene]dichloropalladium(II) catalyst (0.028 g, 0.04 mmol) were combined in a 2-5 mL Biotage microwave vial which was capped and purged with N₂ for 10 min. To this a mixture of IPA:H₂O (4.5 mL, 2:1 ) and tert-butylamine (0.22 mL, 2.10 mmol) were added and the vial was flushed again under N₂ for 10 min. The vial was subjected to microwave irradiation for 45 min at 160° C. After cooling, the solvent was concentrated under reduced pressure. The suspension of the crude 4 -(2 -fluoro-1 -cyanophenyl-5 -yl)-8 -methyl-α-carboline in ethanol (4.5 mL) and a magnetic stirrer were introduced in 2-5 mL Biotage microwave vial which was capped and purged with N₂ for 10 min. Hydrazine hydrate (0.1 mL, 2.95 mmol) was added, and the vial was subjected to microwave irradiation for 30 min at 165° C. After cooling, the reaction mixture was filtered and washed with ethanol (20 mL) and the filtrates were evaporated in vacuo. The solid residue was purified by flash chromatography (SiO₂, dichloromethane—dichloromethane:MeOH 90:10 ) to afford the titled compound a white powder (0.014 g, 0.04 mmol, 11 %). ¹H NMR (400 MHz, DMSO-d₆ ) δ 2.56 (s, 3 H, CH₃ ), 5.46 (s, 2 H, NH₂ ), 6.83-6.92 (m, 1 H), 7.09 (d, J=5.0 Hz, 1 H), 7.19 (d, J=7.2 Hz, 1 H), 7.37 (d, J=7.6 Hz, 1 H), 7.44 (d, J=8.5 Hz, 1 H), 7.53 (d, J=8.5 Hz, 1 H), 8.01 (s, 1 H), 8.44 (d, J=5.0 Hz, 1 H), 11.62 (s, 1 H, NH), 11.86 (s, 1 H, NH). HRMS (ESI) found 314.1401 (M+H)⁺calculated for C₁₉H₁₆N₅ 314.1400.

### 5 -(8 -Isopropyl-9 H-pyrido[2,3 -b]indol-5 -yl)-1 H-indazol-3 -amine SU1259

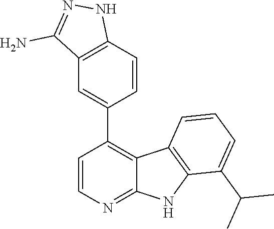 [SMILES: CC(C)c1cccc2c1[nH]c1nccc(-c3ccc4[nH]nc(N)c4c3)c12]

5 -Chloro-8 -isopropyl-9 H-pyrido[2,3 -b]indole (0.020 g, 0.08 mmol), 5 -(4,4,5,5 -tetramethyl-1,3,2 -dioxaborolan-2 -yl)-1 H-indazol-3 -amine (0.021 g, 0.11 mmol), [1,1 ′-bis(di-tert-butylphosphino)ferrocene]dichloropalladium(II) catalyst (0.003 g, 0.005 mmol) and a magnetic stirrer bar were added in a 2-5 mL Biotage microwave vial which was sealed with an aluminium crimp cap fitted with a disposable PTFE/silicon septum, and flushed with nitrogen. Then EtOH (0.3 mL) was added, and the resulting mixture was purged with nitrogen for 10 min before adding 1 M K₃ PO₄ (aqueous solution, 0.16 mL). The mixture was purged again with nitrogen for 10 min and heated to 100° C. for 24 hours. After cooling water was added to the reaction mixture and the solid that precipitated was filtered and dried in vacuo. The solid was solubilised with EtOAc:MeOH 95:5 (10 mL) and filtered, the filtrates were evaporated to obtain the title compound as beige solid (0.021 g, 0.06 mmol, 77 %). ¹H NMR (400 MHz, DMSO-d₆ ) δ ¹H NMR (400 MHz, DMSO-d₆ ) δ 1.32 (d, J=6.7 Hz, 6 H), 3.53-3.65 (m, 1 H), 5.47 (s, 2 H), 6.95-6.99(m, 1 H), 7.09 (d, J=4.8 Hz, 1 H), 7.29 (d, J=7.3 Hz, 1 H), 7.38 (d, J=7.6 Hz, 1 H), 7.42 (d, J=8.0 Hz, 1 H), 7.52 (d, J=7.3 Hz, 1 H), 8.03 (s, 1 H), 8.44 (d, J=4.8 Hz, 1 H), 11.61 (s, 1 H), 11.90 (s, 1 H). HRMS (ESI) found 342.1711 (M+H)⁺calculated for C₁₉H₁₆N₅ 342.1713

### 5 -(6,7,8,9 -Tetrahydro-5 H-pyrido[2,3 -b]indol-4 -yl)-1 H-indazol-3 -amine SU1266

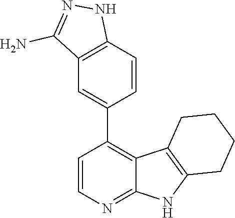 [SMILES: Nc1n[nH]c2ccc(-c3ccnc4[nH]c5c(c34)CCCC5)cc12]

4 -Bromo-6,7,8,9 -tetrahydro-5 H-pyrido[2,3 -b]indole (0.497 mg, 1.98 mmol), 5 -(4,4,5,5 -tetramethyl-1,3,2 -dioxaborolan-2 -yl)-1 H-indazol-3 -amine (0.718 g, 2.77 mmol), [1,1 ′-bis(di-tert-butylphosphino)ferrocene]dichloropalladium(II) catalyst (0.039 g, 0.059 mmol) and a magnetic stirrer bar were introduced in a 10-20 mL Biotage microwave vial which was sealed with an aluminium crimp cap fitted with a disposable PTFE/silicon septum, and flushed with nitrogen. Then EtOH (8 mL) was added, and the resulting mixture was purged with nitrogen for 10 min before adding 1 M K₃ PO₄ (aqueous solution, 4 mL). The mixture was purged again with nitrogen for 10 min and heated to 100° C. overnight. After cooling water was added to the reaction mixture and the solid that precipitated was filtered and dried in vacuo. The crude was purified by flash chromatography (SiO₂, dichloromethane—dichloromethane:MeOH, 90:10 ) to obtain the title compound as a beige solid (0.414 g, 1.36 mmol, 69 %). ¹H NMR (400 MHz, DMSO-d₆ ) δ 1.50-1.66 (m, 2 H), 1.77-1.81 (m, 2 H), 2.20 (t, J=5.2 Hz, 2 H), 2.71 (t, J=5.8 Hz, 2 H), 5.43 (s, 2 H), 6.86 (d, J=4.9 Hz, 1 H), 7.29 (d, J=8.6 Hz, 1 H), 7.33 (d, J=8.6 Hz, 1 H), 7.78 (s, 1 H), 8.07 (d, J=4.9 Hz, 1 H), 11.27 (s, 1 H), 11.46 (s, 1 H). HRMS (ESI) found 304.1557 (M+H)⁴ calculated for C₁₈H₁₈N₅ 304.1552.

### Route to SU1282

### 4 -Bromo-5,6,7,8,9,10 -hexahydrocyclohepta[4,5 ]pyrrolo[2,3 -b]pyridine

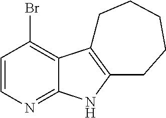 [SMILES: Brc1ccnc2[nH]c3c(c12)CCCCC3]

4 -Bromo-2 -(2 -cycloheptylidenehydrazinyl)pyridine (0.59 g, 2.10 mmol), p-toluenesulfonic acid monohydratate (0.8 g, 4.20 mmol) were introduced in 100 mL round bottom flask and the mixture was heated at 175° C. for 24 h. After cooling, the solution was neutralised with saturated solution of Na₂ CO₃ and it was extracted with EtOAc (3×20 mL). The organic layers were dried under MgSO₄ and the solvent evaporated in vacuo. The crude was purified by flash chromatography (SiO₂, hexane—hexane:EtOAc, 70:30 ) to obtain the title compound as white solid (0.072 g, 0.27 mmol, 13 %). ¹H NMR (400 MHz, DMSO-d₆ ) δ 1.64-1.72 (m, 4 H), 1.80 -1.86 (m, 2 H), 2.78-2.86 (m, 2 H), 3.09-3.18 (m, 2 H), 7.18 (d, J=5.1 Hz, 1 H), 7.87 (d, J=5.1 Hz, 1 H), 11.66 (s, 1 H). HRMS found 265.0331 (M+H)⁺ calculated for C₁₂H₁₄BrN₂ 265.0335.

### 5 -(5,6,7,8,9,10 -Hexahydrocyclohepta[4,5 ]pyrrolo[2,3 -b]pyridin-4 -yl)-1 H-indazol-3 -amine SU1282

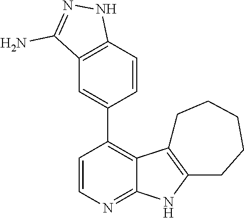 [SMILES: Nc1n[nH]c2ccc(-c3ccnc4[nH]c5c(c34)CCCCC5)cc12]

4 -Bromo-5,6,7,8,9,10 -hexahydrocyclohepta[4,5 ]pyrrolo[2,3 -b]pyridine (0.05 g, 0.19 mmol), 5 -(4,4,5,5 -tetramethyl-1,3,2 -dioxaborolan-2 -yl)-1 H-indazol-3 -amine (0.068 g, 0.264 mmol), [1,1 ′-bis(di-tert-butylphosphino)ferrocene]dichloropalladium(II) catalyst (0.004 g, 0.006 mmol) and a magnetic stirrer bar were added in a 2-5 mL Biotage microwave vial which was sealed with an aluminium crimp cap fitted with a disposable PTFE/silicon septum, and flushed with nitrogen. Then EtOH (0.76 mL) was added, and the resulting mixture was purged with nitrogen for 10 min before adding 1 M K₃ PO₄ (aqueous solution, 0.38 mL). The mixture was purged again with nitrogen for 10 min and heated to 100° C. overnight. After cooling water was added to the reaction mixture and the solid that precipitated was filtered and dried in vacuo. The crude was purified by flash chromatography (SiO₂, dichloromethane—dichloromethane:MeOH, 95:5 ) to obtain the title compound as a beige solid (0.010 g, 0.031 mmol, 63 %). ¹H NMR (400 MHz, DMSO-d₆ ) δ 1.40-1.50 (m, 2 H), 1.65-1.70 (m, 2 H), 1.70-1.74 (m, 2 H), 2.31-2.34 (m, 2 H), 2.81-2.84 (m, 2 H), 5.41 (s, 2 H), 6.81 (d, J=5.0 Hz, 1 H), 7.27-7.30 (m, 2 H), 7.76 (s, 1 H), 8.05 (d, J=5.0 Hz, 1 H), 11.36 (s, 1 H), 11.47 (s, 1 H). HRMS (ESI) found 318.1707 (M+H)⁺calculated for C₁₉H₂₀N₅ 318.1713 .

### tert-Butyl-4 -(3 -amino-1 H-indazol-5 -yl)-5,7,8,9 -tetrahydro-6 H-pyrrolo[2,3 -b:4,5 -c]dipyridine-6 -carboxylate SU1533

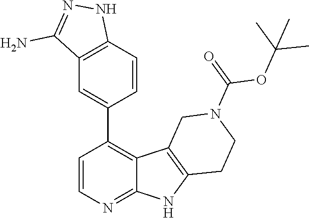 [SMILES: CC(C)(C)OC(=O)N1CCc2[nH]c3nccc(-c4ccc5[nH]nc(N)c5c4)c3c2C1]

A 2-5 mL Biotage MW tube was charged with tert-butyl 4 -chloro-5,7,8,9 -tetrahydro-6 H-pyrrolo[2,3 -b:4,5 -c′]dipyridine-6 -carboxylate (139 mg, 0.45 mmol, 1 eq.), 5 -(4,4,5,5 -tetramethyl-1,3,2 -dioxaborolan-2 -yl)-1 H-indazol-3 -amine (140 mg, 0.54 mmol, 1.2 eq.), [1,1 ′-bis(di-tert-butylphosphino)ferrocene]dichloropalladium(II) catalyst (14.7 mg, 0.0225 mmol, 5 mol %) and ethanol (1.8 mL, oxygen-free). The resulting suspension was purged with nitrogen for 5 minutes before adding 1 M aq. K3 PO4 (0.9 mL). The reaction mixture was heated to 40 degC under a gentle flow of nitrogen for 10 minutes, then to 110 degC for 17 hours. The stirred reaction mixture was then cooled to room temperature, slowly diluted with water (7 mL) and filtered. The filtered solid was then washed with water (3 mL×3 ) and hexane (3 mL×3 ) to give the titled compound as an off-white solid (182 mg, 100 %). ¹H NMR (500 MHz, DMSO) δ 1.18-1.39 (m, 9 H), 2.75-2.82 (m, 2 H), 3.56-3.74 (m, 2 H), 3.98-4.14 (m, 2 H), 5.43 (s, 2 H), 6.94 (d, J=4.9 Hz, 1 H), 7.33 (s, 2 H), 7.79 (s, 1 H), 8.14 (d, J=4.9 Hz, 1 H), 11.43-11.64 (m, 2 H). 2 rotamers observed.LRMS: Calculated for C₂₂H₂₄N₆O₂ 404.20 ; Found: 405.20 [M+H]⁺

### 5 -(6,7,8,9 -Tetrahydro-5 H-pyrrolo[2,3 -b:4,5 -c′]dipyridin-4 -yl)-1 H-indazol-3 -amine SU1541

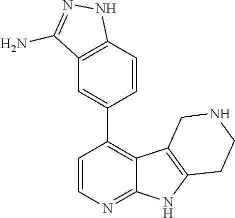 [SMILES: Nc1n[nH]c2ccc(-c3ccnc4[nH]c5c(c34)CNCC5)cc12]

A solution of tert-butyl 4 -(3 -amino-1 H-indazol-5 -yl)-5,7,8,9 -tetrahydro-6 H-pyrrolo[2,3 -b:4,5 -c′]dipyridine-6 -carboxylate (89 mg, 0.22 mmol) in dichloromethane (4 mL) and TFA (168 uL, 2.20 mmol, 10 eq.) was stirred at room temperature overnight. The reaction mixture was then concentrated in vacuo, the residue was treated with 1 M aq. Na2 CO3, extracted with AcOEt and purified by flash chromatography (Silica, 50 g, 1-10 %, MeOH in AcOEt containing 1 % triethylamine) to give the titled compound as a pale yellow solid (16 mg, 24 %). ⁴ H NMR (500 MHz, DMSO) δ 2.70-2.73 (m, 2 H), 2.99-3.05 (m, 2 H), 3.42-3.46 (m, 2 H), 5.44 (s, 2 H), 6.91 (d, J=4.9 Hz, 1 H), 7.29-7.37 (m, 2 H), 7.80 (s, 1 H), 8.11 (d, J=4.9 Hz, 1 H), 11.38 (s, 1 H), 11.48 (s, 1 H). Aliphatic NH not observed LRMS: Calculated for C₁₇H₁₆N₆ 304.14 ; Found: 305.2 [M+H]⁺

### Aryl Substituents at Position 2 of the Azaindole Ring

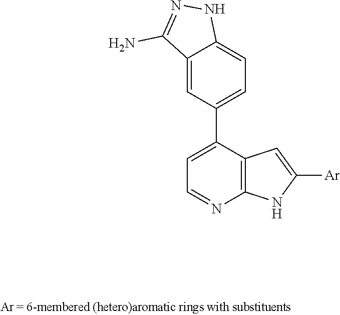

### 5 -(2 -Phenyl-1 H-pyrrolo[2,3 -b]pyridin-4 -yl)-1 H-indazol-3 -amine SU1253

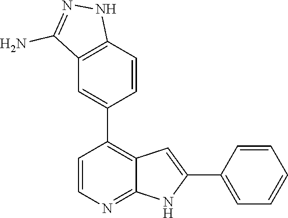 [SMILES: Nc1n[nH]c2ccc(-c3ccnc4[nH]c(-c5ccccc5)cc34)cc12]

To a suspension of 4 -chloro-2 -phenyl-1 H-pyrrolo[2,3 -b]pyridine (0.08 g, 0.35 mmol), 5 -(4,4,5,5 -tetramethyl-1,3,2 -dioxaborolan-2 -yl)-1 H-indazol-3 -amine (0.136 g, 0.525 mmol) and [1,1 ′-bis(di-tert-butylphosphino)ferrocene]dichloropalladium(II) catalyst (0.011 g, 0.0175 mmol) in EtOH/water (1:1; 1.5 mL, degassed under nitrogen) was added K₃ PO₄ (1 M, 0.88 mL) and the reaction mixture was heated to 120° C. for 20 h. The reaction mixture was cooled to room temperature, diluted with EtOAc, washed with water and brine. The organics were concentrated under reduced pressure, diluted into EtOAc, and filtered through a pad of celite. The solution was then concentrated under reduced pressure and purified by column chromatography (100 % Hexane—1/1 Hexane/EtOAc—2/1 EtOAc/Hexane—100 % EtOAc—10 % MeOH/EtOAc) to afford the title compound as an off-white solid (0.05 g, 44 %).¹H NMR (500 MHz, DMSO-D₆ ) δ 5.73 (s, 2 H), 7.17 (d, J=1.9 Hz, 1 H), 7.22 (d, J=8.5 Hz, 1 H), 7.34 -7.40 (m, 3 H), 7.49-7.55 (m, 2 H), 7.74 (s, 1 H), 7.99 (d, J=8.5 Hz, 1 H), 8.25 (m, 2 H), 12.05 (s, 1 H), 12.27 (s, 1 H).

### 5 -(2 -(3 -Ttrifluoromethyl)phenyl)-1 H-pyrrolo[2,3 -b]pyridin-4 -yl)-1 H-indazol-3 -amine SU1297

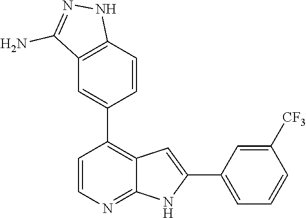 [SMILES: Nc1n[nH]c2ccc(-c3ccnc4[nH]c(-c5cccc(C(F)(F)F)c5)cc34)cc12]

4 -Chloro-2 -(3 -(trifluoromethyl)phenyl)-1 H-pyrrolo[2,3 -b]pyridine (0.100 g, 0.23 mmol), 5 -(4,4,5,5 -tetramethyl-1,3,2 -dioxaborolan-2 -yl)-1 H-indazol-3 -amine (0.128 g, 0.35 mmol), [1,1 ′-bis(di-tert-butylphosphino)ferrocene]dichloropalladium(II) catalyst (0.020 mg, 0.017 mmol) and a magnetic stirrer bar were added in a 2-5 mL MV vial, and flushed with nitrogen. EtOH (4 mL) was added and the resulting mixture was purged with nitrogen for 10 min before adding 1 M K₃ PO₄ (aqueous solution, 0.7 mL) and heated to 100° C. overnight. After cooling, water was added and the solid precipitated was filtered and dried in vacuo. The crude was purified by flash chromatography (SiO₂, hexane:EtOAc 1:1-100 % EtOAc) to obtain the title compound as a beige solid (0.016 g, 0.04 mmol, 17 %). ¹H NMR (500 MHz, DMSO-d₆ ) δ 5.57 (s, 2 H), 7.24 (d, J=5.0 Hz, 1 H), 7.38 (d, J=2.1 Hz, 1 H), 7.45 (d, J=9.0 Hz 2 H), 7.70 (m, 2 H), 7.80 (d, J=9.0 Hz 2 H), 8.30 (m, 3 H), 8.37 (s, 1 H), 12.45 (s, 1 H). LRMS found 394.27 (M+H)⁺calculated for C₂₁H₁₄ F3N₅ 393.12.

### 3 -(4 -(3 -Amino-1 H-indazol-5 -yl)-1 H-pyrrolo[2,3 -b]pyridin-2 -yl)benzonitrile SU1296

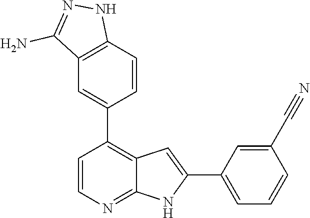 [SMILES: N#Cc1cccc(-c2cc3c(-c4ccc5[nH]nc(N)c5c4)ccnc3[nH]2)c1]

3 -(4 -Chloro-1 H-pyrrolo[2,3 -b]pyridin-2 -yl)benzonitrile (0.100 g, 0.40 mmol), 5 -(4,4,5,5 -tetramethyl-1,3,2 -dioxaborolan-2 -yl)-1 H-indazol-3 -amine (0.155 g, 0.60 mmol), [1,1 ′-bis(di-tert-butylphosphino)ferrocene]dichloropalladium(li) catalyst (0.020 mg, 0.017 mmol) and a magnetic stirrer bar were added in a 2-5 mL MV vial, and flushed with nitrogen. EtOH (4 mL) was added and the resulting mixture was purged with nitrogen for 10 min before adding 1 M K₃ PO₄ (aqueous solution, 0.7 mL) and heated to 100° C. overnight. After cooling water was added and the solid precipitated was filtered and dried in vacuo. The crude was purified by flash chromatography (SiO₂, hexane:EtOAc 1:1-100 % EtOAc) to obtain the title compound as beige solid (0.013 g, 0.04 mmol, 10 %). ¹H NMR (500 MHz, DMSO-d₆ ) δ 5.54 (s, 2 H), 7.22 (d, J=4.5 Hz, 1 H), 7.40 (m, 2 H), 7.67 (t, J=8.0 Hz, 1 H), 7.74 (d, J=8.5 Hz 1 H) 7.80 (d, J=7.0 Hz 1 H), 8.23 (s, 1 H), 8.30 (m, 2 H), 8.48 (s, 1 H), 11.56 (s, 1 H), 12.34 (s, 1 H). LRMS found 351.26 (M+H)+calculated for C₂₁H₁₄N₆ 350.13.

### 3 -(4 -(3 -Amino-1 H-indazol-5 -yl)-1 H-pyrrolo[2,3 -b]pyridin-2 -yl)phenol SU1348

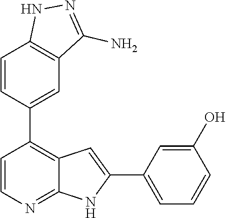 [SMILES: Nc1n[nH]c2ccc(-c3ccnc4[nH]c(-c5cccc(O)c5)cc34)cc12]

3 -(4 -Chloro-1 H-pyrrolo[2,3 -b]pyridin-2 -yl)phenol (0.085 g, 0.35 mmol), 5 -(4,4,5,5 -tetramethyl-1,3,2 -dioxaborolan-2 -yl)-1 H-indazol-3 -amine (0.108 g, 0.42 mmol), [1,1 ′-bis(di-tert-butylphosphino)ferrocene]dichloropalladium(li) catalyst (0.011 mg, 0.017 mmol) and a magnetic stirrer bar were added in a 2-5 mL Biotage microwave vial which was sealed with an aluminium crimp cap fitted with a disposable PTFE/silicon septum, and flushed with nitrogen. Then EtOH (4 mL) was added, and the resulting mixture was purged with nitrogen for 10 min before adding IM K₃ PO₄ (aqueous solution, 0.7 mL). The mixture was purged again with nitrogen for 10 min and heated to 100° C. overnight. After cooling water was added to the reaction mixture and the solid that precipitated was filtered and dried in vacuo. The crude was purified by flash chromatography (SiO₂, hexane:EtOAc 10:90 - EtOAc—EtOAc:MeOH, 90:10 ) to obtain the title compound as brown solid (0.046 g, 39 %). ¹H NMR (400 MHz, DMSO-d₆ ) δ 5.57 (s, 2 H), 7.09 (d, J=2.2 Hz, 1 H), 6.77 (d, J=8.0 Hz, 1 H), 7.19 (d, J=5.3 Hz, 1 H), 7.24 -7.28 (m, 1 H), 7.34 (s, 1 H), 7.38-7.44 (m, 2 H), 7.71 (dd, J=8.5, 2.1 Hz, 1 H), 8.24-8.25 (m, 2 H), 9.56 (s, 1 H), 11.56 (s, 1 H), 12.16 (s, 1 H). HRMS (ESI) found 314.1401 (M+H)+calculated for C₁₉H₁₆N₅ 314.1400.3 -(4 -(3 -Amino-1 H-indazol-5 -yl)-1 H-pyrrolo[2,3 -b]pyridin-2 -yl)benzoic acid SU1303

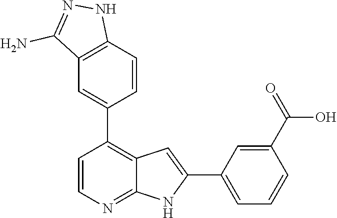 [SMILES: Nc1n[nH]c2ccc(-c3ccnc4[nH]c(-c5cccc(C(=O)O)c5)cc34)cc12]

3 -(4 -Chloro-1 H-pyrrolo[2,3 -b]pyridin-2 -yl)benzoic acid (0.100 g, 0.40 mmol), 5 -(4,4,5,5 -tetramethyl-1,3,2 -dioxaborolan-2 -yl)-1 H-indazol-3 -amine (0.155 g, 0.60 mmol), [1,1 ′-bis(di-tert-butylphosphino)ferrocene]dichloropalladium(II) catalyst (0.020 mg, 0.017 mmol) and a magnetic stirrer bar were added in a 2-5 mL MV vial, and flushed with nitrogen. EtOH (4 mL) was added, and the resulting mixture was purged with nitrogen for 10 min before adding 1 M K₃ PO4 (aqueous solution, 0.7 mL) and heated to 100° C. overnight. After cooling water was added and the solid precipitated was filtered and dried in vacuo. The crude was purified by flash chromatography (SiO₂, hexane:EtOAc 1:1-100 % EtOAc) to obtain the title compound as beige solid (0.037 g, 0.10 mmol, 25 %). ¹H NMR (500 MHz, DMSO-d₆ ) δ 5.54 (s, 2 H), 7.26 (m, 2 H), 7.49 (d, J=8.7 Hz, 1 H), 7.61 (t, J=7.8 Hz, 1 H), 7.85 (d, J=8.5 Hz 1 H), 7.92 (d, J=7.0 Hz, 1 H), 8.23 (d, J=8.1 Hz, 1 H), 8.30 (d, J=5.0 Hz, 1 H), 8.34 (s, 1 H) 8.53 (s, 1 H), 12.48 (s, 1 H). LRMS found 370.2 (M+H)⁺calculated for C₂₁H₁₅N₆O₂ 369.12.

### 3 -(4 -(3 -Amino-1 H-indazol-5 -yl)-1 H-pyrrolo[2,3 -b]pyridin-2 -yl)benzamide SU1294

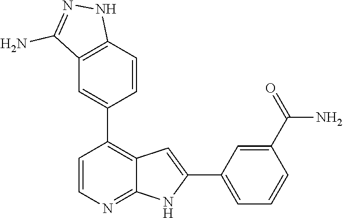 [SMILES: NC(=O)c1cccc(-c2cc3c(-c4ccc5[nH]nc(N)c5c4)ccnc3[nH]2)c1]

3 -(4 -Chloro-1 H-pyrrolo[2,3 -b]pyridin-2 -yl)benzamide (0.100 g, 0.40 mmol), 5 -(4,4,5,5 -tetramethyl-1,3,2 -dioxaborolan-2 -yl)-1 H-indazol-3 -amine (0.155 g, 0.60 mmol), [1,1 ′-bis(di-tert-butylphosphino)ferrocene]dichloropalladium(II) catalyst (0.020 mg, 0.017 mmol) and a magnetic stirrer bar were added in a 2-5 mL MV vial, and flushed with nitrogen. EtOH (4 mL) was added, and the resulting mixture was purged with nitrogen for 10 min before adding 1 M K₃ PO₄ (aqueous solution, 0.7 mL) and heated to 100° C. overnight. After cooling water was added and the solid precipitated was filtered and dried in vacuo. The crude was purified by flash chromatography (SiO₂, hexane:EtOAc 1:1-100 % EtOAc) to obtain the title compound as beige solid (0.026 g, 0.07 mmol, 17 %). ¹H NMR (500 MHz, DMSO-d₆ ) δ 5.54 (s, 2 H), 7.26 (m, 2 H), 7.49 (d, J=8.7 Hz, 1 H), 7.61 (t, J=7.8 Hz, 1 H), 7.85 (d, J=8.5 Hz 1 H), 7.92 (d, J=7.0 Hz, 1 H), 8.23 (d, J=8.1 Hz, 1 H), 8.30 (d, J=5.0 Hz, 1 H), 8.34 (s, 1 H) 8.53 (s, 1 H), 12.48 (s, 1 H). LRMS found 370.2 (M+H)+calculated for C₂₁H₁₅N₆O₂ 369.12.

### 3 -(4 -(3 -Amino-1 H-indazol-5 -yl)-1 H-pyrrolo[2,3 -b]pyridin-2 -yl)-N-(2 -methoxyethyl)benzamide SU1316

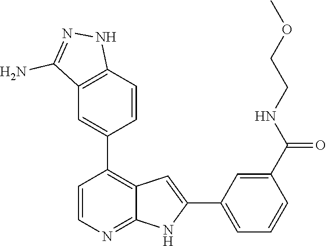 [SMILES: COCCNC(=O)c1cccc(-c2cc3c(-c4ccc5[nH]nc(N)c5c4)ccnc3[nH]2)c1]

3 -(4 -(3 -Amino-1 H-indazol-5 -yl)-1 H-pyrrolo[2,3 -b]pyridin-2 -yl)benzoic acid (SU1303 ) (0.05 g, 0.1 mmol) and 2 -methoxyethan-1 -amine (17 ul, 0.2 mmol) were placed in a 50 ml RBF, DMF (5 ml) was then added and the solution allowed to stir for 5 min, HCTU (123 mg, 0.3 mmol) and trimethylamine (42 ul, 0.3 mmol) were then added and the solution allowed to stir at room temperature overnight. The solvent was then removed under high vacuum and the resulting residue purified by flash chromatography (10 % methanol in EtOAc with 1 % triethylamine) to give the desired product as an off white solid. (105 mg, 88 %), ¹H NMR (500 MHz, DMSO-d₆ ) δ 2.08 (s, 3 H), 3.28 (bs, 2 H), 3.48 (bs, 2 H), 5.54 (s, 2 H), 7.24 (m, 2 H), 7.49 (d, J=8.7 Hz, 1 H), 7.57 (t, J=7.8 Hz, 1 H), 7.79 (d, J=8.5 Hz 1 H), 8.12 (d, J=7.0 Hz, 1 H), 8.31 (d, J=8.1 Hz, 1 H), 8.44 (d, J=5.0 Hz, 1 H), 8.59 (s, 1 H), 12.35 (s, 1 H). LRMS found 427.20 (M+H)+calculated for C₂₄H₂₂N₆O₂ 426.18

### 3 -(4 -(3 -Amino-1 H-indazol-5 -yl)-1 H-pyrrolo[2,3 -b]pyridin-2 -yl)-N-(2 -(piperidin-1 -yl)ethyl)benzamide SU1318

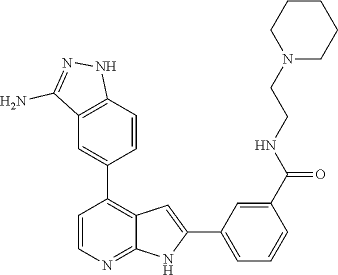 [SMILES: Nc1n[nH]c2ccc(-c3ccnc4[nH]c(-c5cccc(C(=O)NCCN6CCCCC6)c5)cc34)cc12]

3 -(4 -(3 -Amino-1 H-indazol-5 -yl)-1 H-pyrrolo[2,3 -b]pyridin-2 -yl)benzoic acid (SU1303 ) (0.05 g, 0.1 mmol) and 2 -(piperidin-1 -yl)ethan-1 -amine (17 ul, 0.2 mmol) were placed in a 50 ml RBF, DMF (5 ml) was therein added and the solution allowed to stir for 5 min, HCTU (123 mg, 0.3 mmol) and trimethylamine (42 ul, 0.3 mmol) were then added and the solution allowed to stir at room temperature overnight. The solvent was then removed under high vacuum and the resulting residue purified by flash chromatography (10 % methanol in EtOAc with 1 % triethylamine) to give the desired product as a pale yellow solid. (95 mg, 72 %),_H NMR (500 MHz, DMSO-d₆ ) δ 1.65 (m, 4 H), 1.84 (d, J=14.5 Hz, 2 H) 2.92 (m, 2 H), 3.26 (m, 2 H), 3.54 (m, 2 H), 3.67 (m, 2 H), 5.54 (s, 2 H), 7.23 (m, 2 H), 7.47 (d, J=8.7 Hz, 1 H), 7.61 (t, J=7.8 Hz, 1 H), 7.80 (m, 2 H), 8.16 (d, J=7.0 Hz, 1 H), 8.30 (d, J=8.1 Hz, 1 H), 8.42 (s, 1 H), 8.78 (t, J=7.8 Hz, 1 H), 9.01 (s, 1 H), 12.30 (s, 1 H). LRMS found 480.13 (M+H)⁺calculated for C₂₉H₂₉N₇O 479.24

### Route to SU1312

### 3 -(4 -Chloro-1 H-pyrrolo[2,3 -b]pyridin-2 -yl)phenyl)(4 -methylpiperazin-1 -yl)methanone

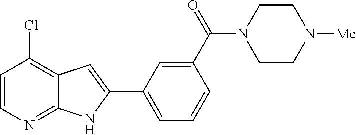 [SMILES: CN1CCN(C(=O)c2cccc(-c3cc4c(Cl)ccnc4[nH]3)c2)CC1]

A suspension of 4 -chloro-2 -iodo-7 -azaindole (0.262 g, 0.94 mmol), (3 -(4 -methylpiperazine-1 -carbonyl)phenylboronic acid (0.353 g, 1.08 mmol), K₂ CO₃ (0.588 g, 4.25 mmol) and Bis(triphenylphosphine) palladium(II) chloride (0.054 g, 0.077 mmol) in 1,4 -dioxane (6 mL) and water (2 mL) was degassed under nitrogen. The reaction mixture was heated to 100° C. for 20 h. The reaction mixture was cooled to room temperature and diluted with EtOAc, washed with water, aqueous saturated NaHCO₃, brine and concentrated under reduced pressure. The resulting solid was triturated with boiling hexane, hot filtered through a pad of celite, concentrated under reduced pressure and dried to afford the title compound as light solid (0.308 g, 0.87 mmol, 93 %). ¹H NMR (DMSO-d₆ ): δ 2.21 (s, 3 H), 2.23 (br s, 2 H), 2.39 (br s, 2 H), 3.36 (br s, 2 H), 3.66 (br s, 2 H), 7.11 (s, 1 H), 7.22 (d, J=4.0 Hz, 1 H), 7.37 (d, J=6.0 Hz, 1 H), 7.55 (t, J=6.2 Hz, 1 H), 8.00 (s, 1 H), 8.08 (d, J=6.8 Hz, 1 H), 8.19 (d, J=4.0 Hz, 1 H), 12.57 (br s, 1 H).

### (3 -(4 -(3 Amino-1 H-indazol-5 -yl)-1 H-pyrrolo[2,3 -b]pyridin-2 -yl)phenyl)(4 -methylpiperazin-1 -y)methanone SU1312

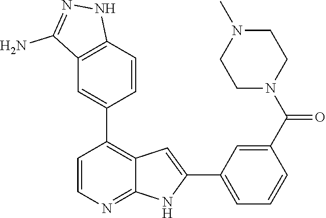 [SMILES: CN1CCN(C(=O)c2cccc(-c3cc4c(-c5ccc6[nH]nc(N)c6c5)ccnc4[nH]3)c2)CC1]

To a suspension of 3 -(4 -chloro-1 H-pyrrolo[2,3 -b]pyridin-2 -yl)phenyl)(4 -methylpiperazin-1 -yl)methanone (0.111 g, 0.31 mmol), 5 -(4,4,5,5 -tetramethyl-1,3,2 -dioxaborolan-2 -yl)-1 H-indazol-3 -amine (0.138 g, 0.53 mmol) and [1,1 ′-bis(di-tert-butylphosphino)ferrocene]dichloropalladium(II) catalyst (0.056 g, 0.086 mmol) in EtOH (3.0 mL, degassed under nitrogen) was added K₃ PO₄ (1 M, 1.2 mL) and the reaction mixture was heated to 100° C. for 16 h. The reaction mixture was cooled to room temperature and the resulting residue was taken up in EtOAc, washed with water, aqueous saturated NaHCO₃ and brine. The organics were combined and concentrated under reduced pressure. The resulting solid was washed with hot dichloromethane to afford the title compound as a brown solid (0.1057 g, 75 %). ¹H NMR (DMSO-d₆ ): δ 2.20 (s, 3 H), 2.30 (m, 2 H), 2.41 (m, 2 H), 3.65 (m, 2 H), 5.58 (s, 2 H), 7.21 (d, J=5.2 Hz, 1 H), 7.28 (d, J=2.0 Hz, 1 H), 7.40 (m, 1 H), 7.54 (t, J=7.8 Hz, 1 H), 7.73 (m, 1 H), 8.00 (s, 1 H), 8.06 (m, 1 H), 8.26 (s, 1 H), 8.28 (d, J=4.8 Hz, 1 H), 11.59 (br s, 1 H), 12.29 (br s, 1 H). HRMS: For C₂₆H₂₆ON₇ requires 452.2193 found 452.2191 .(M+H)⁺

### Route to SU1374

### 4 -Chloro-2 -(3 -(methylsulfonyl)phenyl)-1 H-pyrrolo[2,3 -b]pyridine

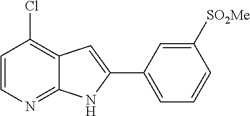 [SMILES: CS(=O)(=O)c1cccc(-c2cc3c(Cl)ccnc3[nH]2)c1]

A suspension of 4 -chloro-2 -iodo-7 -azaindole (0.343 g, 1.23 mmol), (3 -(methylsulfony)phenyl)boronic acid (0.324 g, 1.62 mmol), K₂ CO₃ (0.435 g, 3.15 mmol) and bis(triphenylphosphine) palladium(II) chloride (0.080 g, 0.11 mmol) in 1,4 -dioxane (3 mL) and water (2 mL) was degassed under nitrogen. The reaction mixture was heated to 100° C. for 20 h. The reaction mixture was cooled to room temperature and extracted into EtOAc, washed with water, aqueous saturated NaHCO₃ and brine. Organics were filtered through a pad of celite and concentrated under reduced pressure. The resulting solid was purified column chromatography (100 % Hexane—1/1 Hexane/EtOAc—2/1 EtOAc/Hexane—100 % EtOAc) to afford the title compound as an off-white solid (0.23 g, 0.76 mmol, 62 %). ¹H NMR (DMSO-d₆ ): δ3.33 (s, 3 H), 7.24 (s, 1 H), 7.26 (dd, J=5.0 Hz, 1 H), 7.77 (t, J=7.8 Hz, 1 H), 7.92 (dt, J=1.2, 8.2 Hz, 1 H), 8.24 (d, J=5.3 Hz, 1 H), 8.35 (dt, J=1.2, 8.2 Hz, 1 H), 8.56 (t, J=1.6 Hz, 1 H), 12.73 (br s, 1 H). LRMS: MS (+ve)=307.13.

### 5 -(2 -(3 -(Methylsulfonyl)phenyl)-1 H-pyrrolo[2,3 -b]pyridin-4 -yl)-1 H-indazol-3 -amine SU1374

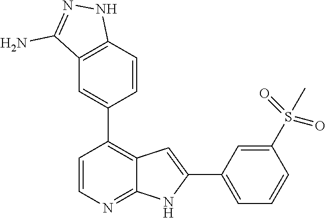 [SMILES: CS(=O)(=O)c1cccc(-c2cc3c(-c4ccc5[nH]nc(N)c5c4)ccnc3[nH]2)c1]

To a suspension of 4 -chloro-2 -(3 -(methylsulfonyl)phenyl)-1 H-pyrrolo[2,3 -b]pyridine (0.12 g, 0.40 mmol), 5 -(4,4,5,5 -tetramethyl-1,3,2 -dioxaborolan-2 -yl)-1 H-indazol-3 -amine (0.170 g, 0.66 mmol) and [1,1 ′-bis(di-tert-butylphosphino)ferrocene]dichloropalladium(II) catalyst (0.029 g, 0.04 mmol) in EtOH (3.0 mL, degassed under nitrogen) was added K₃ PO₄ (1 M, 1.2 mL) and the reaction mixture was heated to 120° C. for 20 h. The reaction mixture was cooled to room temperature, diluted with EtOAc, washed with water and brine. The organics were concentrated under reduced pressure, diluted into EtOAc, and filtered through a pad of celite. The solution was then concentrated under reduced pressure and purified by column chromatography (100 % Hexane—1/1 Hexane/EtOAc—2/1 EtOAc/Hexane—100 % EtOAc—10 % MeCOH/EtOAc) to afford the title compound as an off-white solid (0.038 g, 0.09 mmol, 23 %). ¹H NMR (DMSO-d₆ ): δ 3.31 (s, 3 H), 7.23 (d, J=4.0 Hz, 1 H), 7.37 (d, J=1.6 Hz, 1 H), 7.42 (d, J=6.8 Hz, 1 H), 7.74 (m, 2 H), 7.87 (d, J=6.4 Hz, 1 H), 8.25 (s, 1 H), 8.32 (m, 2 H), 8.27 (t, J=1.2 Hz, 1 H), 11.57 (br s, 1 H), 12.43 (br s, 1 H). HRMS: For C₂₁H₁₈02N₅ S requires 404.1176 found 404.1172 . (M+H)⁺

### Route to SU1311

### 4 -((3 -(4 -Chloro-1 H-pyrrolo[2,3 -b]pyridin-2 -yl)phenyl)sulfonyl)morpholine

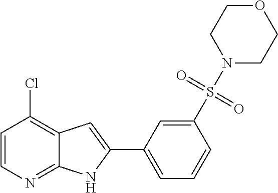 [SMILES: O=S(=O)(c1cccc(-c2cc3c(Cl)ccnc3[nH]2)c1)N1CCOCC1]

A suspension of 4 -chloro-2 -iodo-7 -azaindole (0.292 g, 1.05 mmol), (3 -(morpholinosulfonyl)phenylboronic acid (0.411 g, 1.52 mmol), K₂ COₛ (0.598 g, 4.33 mmol) and Bis(triphenylphosphine) palladium(II) chloride (0.071 g, 0.1 mmol) in 1,4 -dioxane (6 mL) and water (2 mL) was degassed under nitrogen. The reaction mixture was heated to 100° C. for 18 h. The reaction mixture was cooled to room temperature and diluted with EtOAc, washed with water, aqueous saturated NaHCO₃, brine and concentrated under reduced pressure. The resulting solid was triturated with boiling MeOH, hot filtered and dried to afford the title compound as brown solid (0.075 g, 0.20 mmol, 19 %).¹H NMR (DMSO-d₆): 5 2.95 (m, 4 H), 3.65 (m, 4 H), 7.23 (d, J=2.0 Hz, 1 H), 7.25 (d, J=5.2 Hz, 1 H), 7.77 (m, 4 H), 8.23 (d, J=4.8 Hz, 1 H), 8.36 (m, 1 H), 12.83 (br s, 1 H).

### 5 -(2 -(3 -(Morpholinosulfonyl)phenyl)-1 H-pyrrolo[2,3 -b]pyridin-4 -yl)-1 H-indazol-3 -amine SU1311

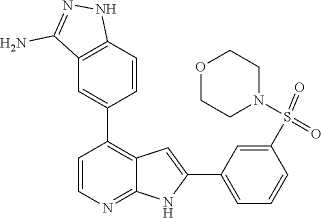 [SMILES: Nc1n[nH]c2ccc(-c3ccnc4[nH]c(-c5cccc(S(=O)(=O)N6CCOCC6)c5)cc34)cc12]

To a suspension of 4 -((3 -(4 -chloro-1 H-pyrrolo[2,3 -b]pyridin-2 -yl)phenyl)sulfonyl)morpholine (0.070 g, 0.19 mmol), 5 -(4,4,5,5 -tetramethyl-1,3,2 -dioxaborolan-2 -yl)-1 H-indazol-3 -amine (0.074 g, 0.29 mmol) and [1,1 ′-bis(di-tert-butylphosphino)ferrocene]dichloropalladium(II) catalyst (0.038 g, 0.058 mmol) in EtOH (3.0 mL, degassed under nitrogen) was added K₃ PO₄ (1 M, 1.2 mL) and the reaction mixture was heated to 100° C. for 18 h. The reaction mixture was cooled to room temperature and the resulting residue was taken up in EtOAc, washed with water, aqueous saturated NaHCO₃ and brine. The organics were combined and concentrated under reduced pressure. The resulting solid was suspended in EtOAc and filtered through a pad of celite and concentrated under reduced pressure to afford the title compound as a pale solid (0.0085 g, 0.018 mmol, 10 %). ¹H NMR (DMSO-d₆ ): δ 2.96 (m, 4 H), 3.65 (m, 4 H), 5.55 (br s, 2 H), 7.23 (d, J=4.8 Hz, 1 H), 7.34 (s, 1 H), 7.41 (d, J=8.8 Hz, 1 H), 7.74 (m, 4 H), 8.26 (s, 1 H), 8.32 (m, 2 H), 11.58 (br s, 1 H), 12.53 (br s, 1 H). HRMS: For C₂₄H₂₃O₃N₆ S requires 475.1547 found 475.1548 . (M+H)⁺

### 5 -(2 -(3 -Aminophenyl)-1 H-pyrrolo[2,3 -b]pyridin-4 -yl)-1 H-indazol-3 -amine SU1304

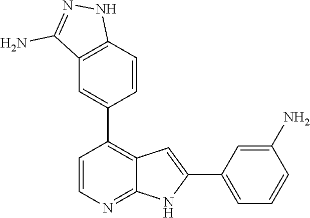 [SMILES: Nc1cccc(-c2cc3c(-c4ccc5[nH]nc(N)c5c4)ccnc3[nH]2)c1]

3 -(4 -Chloro-1 H-pyrrolo[2,3 -b]pyridin-2 -yl)aniline (0.100 g, 0.23 mmol), 5 -(4,4,5,5 -tetramethyl-1,3,2 -dioxaborolan-2 -yl)-1 H-indazol-3 -amine (0.128 g, 0.35 mmol), [1,1 ′-bis(di-tert-butylphosphino)ferrocene]dichloropalladium(II) catalyst (0.020 mg, 0.017 mmol) and a magnetic stirrer bar were added in a 2-5 mL MV vial, and flushed with nitrogen. EtOH (4 mL) was added, and the resulting mixture was purged with nitrogen for 10 min before adding 1 M K₃ PO₄ (aqueous solution, 0.7 mL) and heated to 100° C. overnight. After cooling water was added and the solid precipitated was filtered and dried in vacuo. The crude was purified by flash chromatography (SiO₂, hexane:EtOAc 1:1-100 % EtOAc) to obtain the title compound as beige solid (0.016 g, 0.04 mmol, 17 %). ¹H NMR (500 MHz, DMSO-d₆ ) δ 5.57 (s, 2 H), 7.15 (s, 1 H), 7.26 (d, J=5.0 Hz, 1 H), 7.34 (t, J=8.0 Hz, 1 H), 7.47-7.54 (m, 3 H), 7.84 (d, J=8.0 Hz 2 H), 8.30 (d, J=7.8 Hz, 1 H), 8.37 (s, 1 H), 12.45 (s, 1 H). LRMS found 341.20 (M+H)+calculated for C₂₀H₁₆N₆ 340.14.

### Route to SU1317

### N-(3 -(4 -Chloro-1 H-pyrrolo[2,3 -b]pyridin-2 -yl)phenyl)-3 -methoxypropanamide

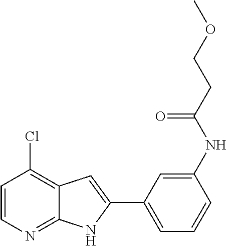 [SMILES: COCCC(=O)Nc1cccc(-c2cc3c(Cl)ccnc3[nH]2)c1]

3 -(4 -Chloro-1 H-pyrrolo[2,3 -b]pyridin-2 -yl)aniline (0.10 g, 0.4 mmol) and 3 -methoxypropanoic acid (37 ul, 0.4 mmol) were placed in a 50 ml RBF, DMF (5 ml) was then added and the solution allowed to stir for 5 min, HCTU (495 mg, 1.2 mmol) and trimethylamine (168 ul, 1.2 mmol) were then added and the solution allowed to stir at room temperature overnight. The solvent was then removed under high vacuum and the resulting residue purified by flash chromatography (10 % methanol in EtOAc with 1 % triethylamine) to give the desired product N-(3 -(4 -chloro-1 H-pyrrolo[2,3 -b]pyridin-2 -yl)phenyl)-3 -methoxypropanamide as a pale yellow solid. (122 mg, 72 %),_¹H NMR (500 MHz, DMSO-d₆ ) δ 2.04 (t, J=8.0 Hz, 2 H), 3.27 (s, 3 H), 3.65 (t, J=8.0 Hz, 2 H), 6.81 (d, J=1.8 Hz, 1 H), 7.22 (d, J=6.5 Hz, 1 H), 7.47 (t, J=10.0 Hz, 1 H), 7.57 (d, J=7.8 Hz, 1 H), 7.66 (d, J=7.8 Hz, 1 H) 7.89 (m, 2 H), 10.04 (s, 1 H), 12.54 (s, 1 H).

LRMS found 244.20 (M+H)+calculated for C₁₃H₁₀ ClN₃ 243.06.

### N-(3 -(4 -(3 -Amino-1 H-indazol-5 -yl)-1 H-pyrrolo[2,3 -b]pyridin-2 -yl)phenyl)-3 -methoxypropanamide SU1317

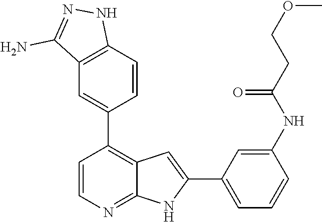 [SMILES: COCCC(=O)Nc1cccc(-c2cc3c(-c4ccc5[nH]nc(N)c5c4)ccnc3[nH]2)c1]

N-(3 -(4 -Chloro-1 H-pyrrolo[2,3 -b]pyridin-2 -yl)phenyl)-3 -methoxypropanamide (0.100 g, 0.23 mmol), 5 -(4,4,5,5 -tetramethyl-1,3,2 -dioxaborolan-2 -yl)-1 H-indazol-3 -amine (0.128 g, 0.35 mmol), [1,1 ′-bis(di-tert-butylphosphino)ferrocene]dichloropalladium(II) catalyst (0.020 mg, 0.017 mmol) and a magnetic stirrer bar were added in a 2-5 mL MV vial, and flushed with nitrogen. EtOH (4 mL) was added, and the resulting mixture was purged with nitrogen for 10 min before adding 1 M K₃ PO₄ (aqueous solution, 0.7 mL) and heated to 100° C. overnight. After cooling water was added and the solid precipitated was filtered and dried in vacuo. The crude was purified by flash chromatography (SiO₂, hexane:EtOAc 1:1-100 % EtOAc) to obtain the title compound as beige solid (0.016 g, 0.04 mmol, 17 %). ¹H NMR (500 MHz, DMSO-d₆ ) δ 2.58 (t, J=8.0 Hz, 2 H), 3.25 (s, 3 H), 3.63 (t, J=8.0 Hz, 2 H), 7.06 (d, J=1.8 Hz, 1 H), 7.25 (d, J=6.5 Hz, 1 H), 7.43 (t, J=10.0 Hz, 1 H), 7.54 (d, J=8.0 Hz, 1 H), 7.60 (d, J=7.8 Hz, 1 H), 7.67 (d, J=7.8 Hz, 1 H), 7.87 (m, 1 H), 8.15 (s, 1 H), 8.32 (d, J=6.50 Hz, 1 H), 10.08 (s, 1 H), 12.45 (s, 1 H).

LRMS found 427.20 (M+H)+calculated for C₂₀H₁₆N₆ 426,18.

### Route to SU1319

### N-(3 -(4 -Chloro-1 H-pyrrolo[2,3 -b]pyridin-2 -yl)phenyl)-3 -(piperidin-1 -yl)propanamide

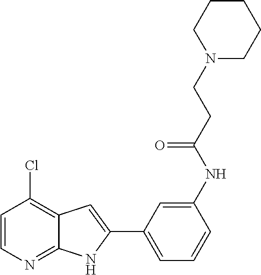 [SMILES: O=C(CCN1CCCCC1)Nc1cccc(-c2cc3c(Cl)ccnc3[nH]2)c1]

3 -(4 -Chloro-1 H-pyrrolo[2,3 -b]pyridin-2 -yl)aniline (0.10 g, 0.4 mmol) and 3 -(piperidin-1 -yl)propanoic acid (37 ul, 0.4 mmol) were placed in a 50 ml RBF, DMF (5 ml) was then added and the solution allowed to stir for 5 min, HCTU (495 mg, 1.2 mmol) and trimethylamine (168 ul, 1.2 mmol) were then added and the solution allowed to stir at room temperature overnight. The solvent was then removed under high vacuum and the resulting residue purified by flash chromatography (10 % methanol in EtOAc with 1 % triethylamine) to give the desired product N-(3 -(4 -chloro-1 H-pyrrolo[2,3 -b]pyridin-2 -yl)phenyl)-3 -(piperidin-1 -yl)propanamide as a pale yellow solid. (122 mg, 72 %) LRMS found 383.20 (M+H)⁺calculated for C₂₁H₂₃ ClN₄O 382.16.

### N-(3 -(4 -(3 -Amino-1 H-indazol-5 -yl)-1 H-pyrrolo[2,3 -b]pyridin-2 -yl)phenyl)-3 -(piperidin-1 -yl)propanamide SU1319

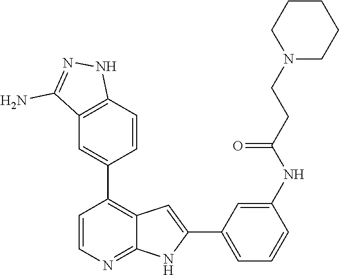 [SMILES: Nc1n[nH]c2ccc(-c3ccnc4[nH]c(-c5cccc(NC(=O)CCN6CCCCC6)c5)cc34)cc12]

N-(3 -(4 -Chloro-1 H-pyrrolo[2,3 -b]pyridin-2 -yl)phenyl)-3 -(piperidin-1 -yl)propanamide (0.100 g, 0.23 mmol), 5 -(4,4,5,5 -tetramethyl-1,3,2 -dioxaborolan-2 -yl)-1 H-indazol-3 -amine (0.128 g, 0.35 mmol), [1,1 ′-bis(di-tert-butylphosphino)ferrocene]dichloropalladium(II) catalyst (0.020 mg, 0.017 mmol) and a magnetic stirrer bar were added in a 2-5 mL MV vial, and flushed with nitrogen. EtOH (4 mL) was added, and the resulting mixture was purged with nitrogen for 10 min before adding 1 M K₃ PO₄ (aqueous solution, 0.7 mL) and heated to 100° C. overnight. After cooling water was added and the solid precipitated was filtered and dried in vacuo. The crude was purified by flash chromatography (SiO₂, hexane:EtOAc 1:1-100 % EtOAc) to obtain the title compound as beige solid (0.016 g, 0.04 mmol, 17 %). ¹H NMR (500 MHz, DMSO-d₆ ) δ 1.65 (m, 4 H), 1.81 (d, J=14.5 Hz, 2 H) 2.84 (m, 4 H), 3.26 (m, 2 H), 3.54 (m, 2 H), 7.10 (d, J=1.8 Hz, 1 H), 7.21 (d, J=6.5 Hz, 1 H), 7.43 (t, J=10.0 Hz, 1 H), 7.57 (d, J=8.0 Hz, 1 H), 7.69 (d, J=7.8Hz, 1 H), 7.67 (d, J=7.8 Hz, 1 H), 8.09 (s, 1 H), 8.29 (m, 2 H), 10.29 (s, 1 H), 12.36 (s, 1 H). LRMS found 479.30 (M+H)⁺calculated for C₂₈ H,₉N₇O 479.24.

### Route to SU 1288

### N-(3 -(4 -Chloro-1 H-pyrrolo[2,3 -b]pyridin-2 -yl)phenyl)methanesulfonamide

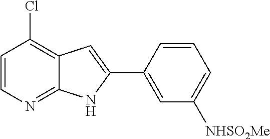

A suspension of 4 -chloro-2 -iodo-7 -azaindole (0.288 g, 1.04 mmol), 3 -(methylsulfonamido)phenylboronic acid (0.254 g, 1.18 mmol), K₂ CO₃ (0.52 g, 3.76 mmol) and Bis(triphenylphosphine) palladium(II) chloride (0.051 g, 0.07 mmol) in 1,4 -dioxane (2 mL) and water (2 mL) was degassed under nitrogen. The reaction mixture was heated to 100° C. for 20 h. The reaction mixture was cooled to room temperature and diluted with EtOAc, washed with water, and concentrated under reduced pressure. Purification by column chromatography (100 % Hexane—1/1 Hexane/EtOAc—100 % EtOAc) afforded the title compound as a pale yellow solid (0.216 g, 0.67 mmol, 64.7 %). ¹H NMR (DMSO-d₆ ): δ 3.10 (s, 3 H), 6.88 (d, J=2.0 Hz, 1 H), 7.21 (m, 2 H), 7.45 (t, J=16.0 Hz, 1 H), 7.73 (m, 2 H), 8.19 (d, J=5.2 Hz, 1 H), 9.88 (br s, 1 H), 12.62 (br s, 1 H). HRMS: For C₁₄H₁₃02N₃ CIS requires 322.0412 found 322.0410.

### N-(3 -(4 -(3 -Amino-1 H-indazol-5 -yl)-1 H-pyrrolo[2,3 -b]pyridin-2 -yl)phenyl)methanesulfonamide SU1288

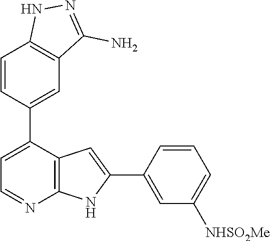

To a suspension of N-(3 -(4 -chloro-1 H-pyrrolo[2,3 -b]pyridin-2 -yl)phenyl)methanesulfonamide (0.185 g, 0.57 mmol), 5 -(4,4,5,5 -tetramethyl-1,3,2 -dioxaborolan-2 -yl)-1 H-indazol-3 -amine (0.197 g, 0.76 mmol) and [1,1 ′-bis(di-tert-butylphosphino)ferrocene]dichloropalladium(II) catalyst (0.048 g, 0.074 mmol) in EtOH (3.0 mL, degassed under nitrogen) was added K₃ PO₄ (1 M, 1.2 mL) and the reaction mixture was heated to 80° C. for 24 h. The reaction mixture was cooled to room temperature and the reaction mixture was diluted with EtOAc, washed with water, aqueous saturated NaHCO₃ and brine. Organics were concentrated under reduced pressure and purified by column chromatography (100 % Hexane—2/1 Hexane/EtOAc—100 % EtOAc—10 % MeOH/EtOAc) and triturated with Et₂O to afford the title compound as an off-white solid (0.022 g, 0.053 mmol, 9.1 %). ¹H NMR (DMSO-d₆): 6 3.09 (s, 3 H), 5.56 (br s, 2 H), 7.07 (d, J=2.0 Hz, 1 H), 7.19 (m, 2 H), 7.42 (q, J=8.3 Hz, 2 H), 7.70 (m, 3 H), 8.23 (s, 1 H), 8.27 (d, J=5.2 Hz, 1 H), 9.83 (br s, 1 H), 11.57 (br s, 1 H), 12.32 (br s, 1 H). HRMS: For C₂₁H₁₉02N₆ S requires 419.1285 found 419.1287 . (M+H)+

### Route to SU1278

### N-(3 -(4 -Chloro-1 H-pyrrolo[2,3 -b]pyridin-2 -yl)phenyl)-4 -methylbenzenesulfonamide

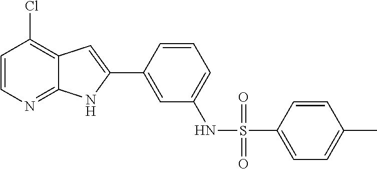 [SMILES: Cc1ccc(S(=O)(=O)Nc2cccc(-c3cc4c(Cl)ccnc4[nH]3)c2)cc1]

A suspension of 4 -chloro-2 -iodo-7 -azaindole (0.289 g, 1.04 mmol), 3 -(p-toluenesulfonamino)phenylboronic acid, pinacol ester (0.432 g, 1.16 mmol), K₂ CO₃ (0.55 g, 3.98 mmol) and Bis(triphenylphosphine) palladium(II) chloride (0.036 g, 0.051 mmol) in 1,4 -dioxane (2 mL) and water (2 mL) was degassed under nitrogen. The reaction mixture was heated to 100° C. for 20 h. The reaction mixture was cooled to room temperature and diluted with EtOAc, washed with water and concentrated under reduced pressure. Purification by column chromatography (100 % Hexane—1/1 Hexane/EtOAc—100 % EtOAc) afforded the title compound as a pale yellow solid (0.3025 g, 0.76 mmol, 73.2 %). ¹H NMR (DMSO-d₆ ): δ 2.33 (s, 3 H), 6.74 (d, J=2.0 Hz, 1 H), 7.07 (m, 1 H), 7.22 (d, J=5.2 Hz, 1 H), 7.35 (m, 3 H), 7.64 (m, 2 H), 7.71 (d, J=8.4 Hz, 2 H), 8.19 (d, J=5.2 Hz, 1 H), 10.36 (br s, 1 H), 12.57 (br s, 1 H).

### N-(3 -(4 -(3 -Amino-1 H-indazol-5 -yl)-1 H-pyrrolo[2,3 -b]pyridin-2 -yl)phenyl)-4 -methylbenzensulfonamide SU1278

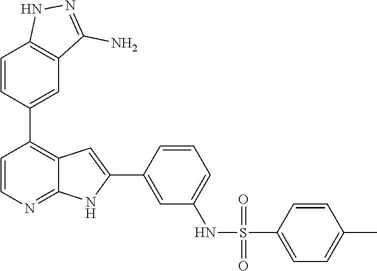 [SMILES: Cc1ccc(S(=O)(=O)Nc2cccc(-c3cc4c(-c5ccc6[nH]nc(N)c6c5)ccnc4[nH]3)c2)cc1]

To a suspension of N-(3 -(4 -chloro-1 H-pyrrolo[2,3 -b]pyridin-2 -yl)phenyl)-4 -methylbenzenesulfonamide (0.224 g, 0.56 mmol), 5 -(4,4,5,5 -tetramethyl-1,3,2 -dioxaborolan-2 -yl)-1 H-indazol-3 -amine (0.158 g, 0.61 mmol) and [1,1 ′-bis(di-tert-butylphosphino)ferrocene]dichloropalladium(II) catalyst (0.041 g, 0.063 mmol) in EtOH (3.0 mL, degassed under nitrogen) was added K₃ PO₄ (1 M, 1.2 mL) and the reaction mixture was heated to 80° C. for 24 h. The reaction mixture was cooled to room temperature and the reaction mixture was diluted with EtOAc, washed with water, aqueous saturated NaHCO₃ and brine. Organics were concentrated under reduced pressure and purified by column chromatography (100 % Hexane—2/1 Hexane/EtOAc—100 % EtOAc—10 % MeOH/EtOAc—10 % MeOH/EtOAc) and triturated Et₂O to afford the title compound as an off-white solid (0.143 g, 0.11 mmol, 28.9 %). ¹H NMR (DMSO-d₆ ): δ 3.33 (s, 3 H), 5.57 (br s, 2 H), 6.92 (d, J=2.0 Hz, 1 H), 7.05 (dd, J=1.2, 8.0 Hz, 1 H), 7.18 (d, J=4.8 Hz, 1 H), 7.32 (t, J=8.0 Hz, 3 H), 7.42 (d, J=8.4 Hz, 1 H), 7.60 (m, 2 H), 7.68 (m, 3 H), 8.22 (s, 1 H), 8.27 (d, J=5.2 Hz, 1 H), 10.32 (s, 1 H), 11.58 (br s, 1 H), 12.25 (br s, 1 H). HRMS: For C₂₇H₂₃02N₆ requires 495.1598 found 495.1592 . (M+H)⁺

### Route to SU1283

### N-(4 -(4 -Chloro-1 H-pyrrolo[2,3 -b]pyridin-2 -yl)phenyl)-4 -methylbenzenesulfonamide

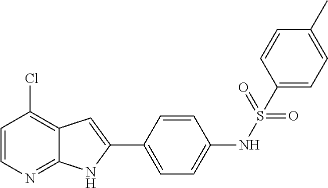 [SMILES: Cc1ccc(S(=O)(=O)Nc2ccc(-c3cc4c(Cl)ccnc4[nH]3)cc2)cc1]

A suspension of 4 -chloro-2 -iodo-7 -azaindole (0.290 g, 1.04 mmol), ⁴ -(p-toluenesulfonamino)phenylboronic acid, pinacol ester (0.450 g, 1.20 mmol), K₂ CO₃ (0.54 g, 3.93 mmol) and Bis(triphenylphosphine) palladium(II) chloride (0.039 g, 0.056 mmol) in 1,4 -dioxane (2 mL) and water (2 mL) was degassed under nitrogen. The reaction mixture was heated to 100° C. for 20 h. The reaction mixture was cooled to room temperature and diluted with EtOAc, washed with water and concentrated under reduced pressure. Purification by column chromatography (100 % Hexane—1/1 Hexane/EtOAc—100 % EtOAc) afforded the title compound as an off-white solid (0.207 g, 0.50 mmol, 50.0 %).¹H NMR (DMSO-d₆ ): δ 2.33 (s, 3 H), 6.87 (d, J=2.4 Hz, 1 H), 7.17 (dd, J=0.8, 6.4 Hz, 3 H), 7.36 (d, J=8.0 Hz, 2 H), 7.69 (dd, J=1.6, 6.4 Hz, 2 H), 7.85 (dd, J=2.0, 6.8 Hz, 2 H), 8.13 (d, J=5.2 Hz, 1 H), 10.47 (br s, 1 H), 12.39 (br s, 1 H).

### N-(4 -(4 -(3 -Amino-1 H-indazol-5 -yl)-1 H-pyrrolo[2,3 -b]pyridin-2 -yl)phenyl)-4 -methylbenzenesulfonamide SU1283

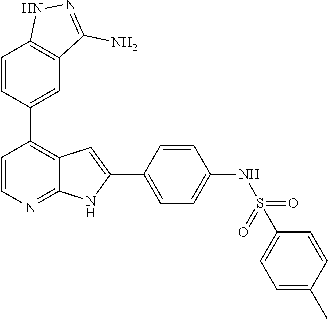 [SMILES: Cc1ccc(S(=O)(=O)Nc2ccc(-c3cc4c(-c5ccc6[nH]nc(N)c6c5)ccnc4[nH]3)cc2)cc1]

To a suspension of N-(4 -(4 -chloro-1 H-pyrrolo[2,3 -b]pyridin-2 -yl)phenyl)-4 -methylbenzenesulfonamide (0.181 g, 0.46 mmol), 5 -(4,4,5,5 -tetramethyl-1,3,2 -dioxaborolan-2 -yl)-1 H-indazol-3 -amine (0.157 g, 0.60 mmol) and [1,1 ′-bis(di-tert-butylphosphino)ferrocene]dichloropalladium(II) catalyst (0.043 g, 0.066 mmol) in EtOH (3.0 mL, degassed under nitrogen) was added K₃ PO₄ (1 M, 1.2 mL) and the reaction mixture was heated to 80° C. for 24 h. The reaction mixture was cooled to room temperature and the reaction mixture was diluted with EtOAc, washed with water, aqueous saturated NaHCO₃ and brine. Organics were concentrated under reduced pressure and purified by column chromatography (100 % Hexane—2/1 Hexane/EtOAc—100 % EtOAc—10 % MeOH/EtOAc) and triturated with Et₂O to afford the title compound as an off-white solid (0.0629 g, 0.13 mmol, 27.9 %). ¹H NMR (DMSO-d₆): a 2.32 (s, 3 H), 5.54 (br s, 2 H), 7.06 (d, J=2.0 Hz, 1 H), 7.15 (d, J=4.0 Hz, 1 H), 7.17 (s, 1 H), 7.37 (t, J=9.2 Hz, 3 H), 7.68 (dd, J=2.0, 8.8 Hz, 3 H), 7.83 (d, J=8.8 Hz, 2 H), 8.20 (s, 1 H), 8.22 (d, J=4.8 Hz, 1 H), 10.40 (br s, 1 H), 11.55 (br s, 1 H), 12.10 (br s, 1 H).

HRMS: For C₂₇H₂₃02N₆ S requires 495.1598 found 495.1592 . (M+H)+

### 2 -(4 -(3 -Amino-1 H-indazol-5 -yl)-1 H-pyrrolo[2,3 -b]pyridin-2 -yl)phenol SU1354

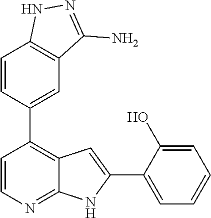 [SMILES: Nc1n[nH]c2ccc(-c3ccnc4[nH]c(-c5ccccc5O)cc34)cc12]

This compound was obtained during the purification process of SU1353 as an off-white solid (0.012 g, 0.034 mmol, 8 %). ¹H NMR (DMSO-d₆ ): δ 5.50 (br s, 2 H), 6.92 (m, 1 H), 6.98 (m, 1 H), 7.17 (m, 2 H), 7.28 (s, 1 H), 7.40 (d, J=8.8 Hz, 1 H), 7.69 (dd, J=1.6, 8.4 Hz, 1 H), 7.85 (dd, J=1.6, 7.6 Hz, 1 H), 8.19 (s, 1 H), 8.23 (d, J=5.2 Hz, 1 H), 10.17 (br s, 1 H), 11.54 (br s, 1 H), 11.67 (br s, 1 H). HRMS: For C₂₀H₁₆ON₅ requires 342.1349 found 342.1346 (M+H)⁺.

### Route to SU1368

### 4 -Chloro-2 -(2 -ethoxyphenyl)-1 H-pyrrolo[2,3 -b]pyridine

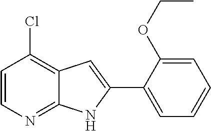 [SMILES: CCOc1ccccc1-c1cc2c(Cl)ccnc2[nH]1]

A suspension of 4 -chloro-2 -iodo-7 -azaindole (0.346 g, 1.24 mmol), (2 -ethoxyphenyl)boronic acid (0.267 g, 1.61 mmol), K₂ CO₃ (0.474 g, 3.43 mmol) and Bis(triphenylphosphine) palladium(II) chloride (0.070 g, 0.10 mmol) in 1,4 -dioxane (3 mL) and water (2 mL) was degassed under nitrogen. The reaction mixture was heated to 100° C. for 20 h. The reaction mixture was cooled to room temperature and extracted into EtOAc, washed with water, aqueous saturated NaHCO₃ and brine. Organics were filtered through a pad of celite and concentrated under reduced pressure. The resulting solid purified by recrystallization using dichloromethane and hexane to afford the title compound as an orange solid (0.21 g, 0.77 mmol, 62 %). ¹H NMR (DMSO-d₆ ): δ 1.46 (t, J=7.0 Hz, 3 H), 4.21 (q, J=6.9 Hz, 2 H), 7.06 (m, 2 H), 7.16 (s, 1 H), 7.19 (d, J=5.2 Hz, 1 H), 7.37 (m, 1 H), 7.89 (dd, J=1.6, 0.6 Hz, 1 H), 8.17 (d, J=5.2 Hz, 1 H), 12.13 (br s, 1 H).

### 5 -(2 -(2 -Ethoxyphenyl)-1 H-pyrrolo[2,3 -b]pyridin-4 -yl)-1 H-indazol-3 -amine SU1368

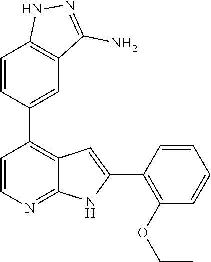 [SMILES: CCOc1ccccc1-c1cc2c(-c3ccc4[nH]nc(N)c4c3)ccnc2[nH]1]

To a suspension of 4 -chloro-2 -(2 -ethoxyphenyl)-1 H-pyrrolo[2,3 -b]pyridine (0.10 g, 0.37 mmol), 5 -(4,4,5,5 -tetramethyl-1,3,2 -dioxaborolan-2 -yl)-1 H-indazol-3 -amine (0.146 g, 0.56 mmol) and [1,1 ′-bis(di-tert-butylphosphino)ferrocene]dichloropalladium(II) catalyst (0.028 g, 0.04 mmol) in EtOH (3.0 mL, degassed under nitrogen) was added K₃ PO₄ (1 M, 1.2 mL) and the reaction mixture was heated to 120° C. for 20 h. The reaction mixture was cooled to room temperature, diluted with EtOAc, washed with water and brine. The organics were concentrated under reduced pressure, diluted into EtOAc, and filtered through a pad of celite. The solution was then concentrated under reduced pressure and purified by column chromatography (100 % Hexane—1/1 Hexane/EtOAc—2/1 EtOAc/Hexane—100 % EtOAc) to afford the title compound as an off-white solid (0.056 g, 0.15 mmol, 41 %). ¹H NMR (DMSO-d₆ ): δ 1.44 (t, J=7.0 Hz, 3 H), 4.18 (q, J=5.5 Hz, 2 H), 5.50 (br s, 2 H), 7.05 (t, J=5.8 Hz, 1 H), 7.14 (d, J=6.4 Hz, 1 H), 7.18 (d, J=5.0 Hz, 1 H), 7.34 (m, 1 H), 7.38 (m, 2 H), 7.72 (dd, J=1.5, 9.0 Hz, 1 H), 7.92 (dd, J=2.0, 8.0 Hz, 1 H), 8.22 (s, 1 H), 8.25 (d, J=5.0 Hz, 1 H), 11.54 (br s, 1 H), 11.81 (br s, 1 H). HRMS: For C₂₂H₂ON₅ requires 370.1662 found 370.1660 . (M+H)⁺.

### 5 -(2 -(2 -Ethylphenyl)-1 H-pyrrolo[2,3 -b]pyridin-4 -yl)-1 H-indazol-3 -amine SU1351

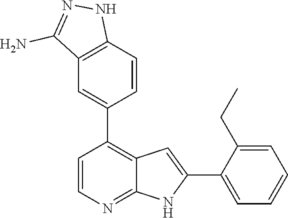 [SMILES: CCc1ccccc1-c1cc2c(-c3ccc4[nH]nc(N)c4c3)ccnc2[nH]1]

A 2-5 mL MW tube was charged with a mixture of 4 -chloro-2 -(2 -ethylphenyl)-1 H-pyrrolo[2,3 -b]pyridine (128 mg, 0.500 mmol, 1 eq.), 5 -(4,4,5,5 -tetramethyl-1,3,2 -dioxaborolan-2 -yl)-1 H-indazol-3 -amine (168 mg, 0.65 mmol, 1.3 eq.), [1,1 ′-bis(di-tert-butylphosphino)ferrocene]dichloropalladium(II) catalyst (16 mg, 0.025 mmol, 5 mol %) in EtOH (2 mL) was stirred under nitrogen before adding 1 M aq. K₃ PO₄ (1 mL). The reaction was heated to 40° C. under a gentle flow of argon for 10 minutes, then to 110° C. for 24 hours. The stirred reaction mixture was then cooled to room temperature, slowly diluted with water (7 mL) and filtered. The filtered solid was then washed with water (7 mL×3 ) and hexane (7 mL×2 ), dissolved with AcOEt (7 mL×2 ) and filtered. The combined filtrates were concentrated in vacuo to give the title compound. ¹H NMR (500 MHz, DMSO) δ 1.11 (t, J=7.5 Hz, 3 H), 2.82 (q, J=7.5 Hz, 2 H), 5.50 (s, 2 H), 6.79 (d, J=1.9 Hz, 1 H), 7.22 (d, J=5.0 Hz, 1 H), 7.28-7.35 (m, 1 H), 7.38 (d, J=7.9 Hz, 3 H), 7.50 (d, J=7.3 Hz, 1 H), 7.70 (dd, J=8.7, 1.5 Hz, 1 H), 8.22 (s, 1 H), 8.26 (d, J=5.0 Hz, 1 H), 11.53 (s, 1 H), 11.94 (s, 1 H).

### Route to SU1330

### 2 -(Benzo[dj[1,3 ]dioxol-5 -yl)-4 -chloro-1 H-pyrrolo[2,3 -b]pyridine

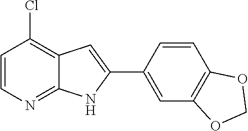 [SMILES: Clc1ccnc2[nH]c(-c3ccc4c(c3)OCO4)cc12]

A suspension of 4 -chloro-2 -iodo-7 -azaindole (0.270 g, 0.97 mmol), benzo[d][1,3 ]dioxol-5 -ylboronic acid (0.175 g, 1.05 mmol), K₂ CO₃ (0.565 g, 4.09 mmol) and Bis(triphenylphosphine) palladium(II) chloride (0.067 g, 0.095 mmol) in 1,4 -dioxane (3 mL) and water (2 mL) was degassed under nitrogen. The reaction mixture was heated to 100° C. for 20 h. The reaction mixture was cooled to room temperature and the resulting precipitate was filtered under reduced pressure, washed with MeOH to afford the title compound as a brown solid (0.214 g, 0.78 mmol, 81 %). ¹H NMR (DMSO-d₆ ): δ 6.08 (s, 2 H), 6.88 (s, 1 H), 7.03 (d, J=8.0 Hz, 1 H), 7.13 (d, J=4.8 Hz, 1 H), 7.51 (dd, J=1.6, 8.0 Hz, 1 H), 7.59 (d, J=2.0 Hz, 1 H), 8.10 (d, J=4.8 Hz, 1 H).

### 5 -(2 -(Benzo[d][1,3 ]dioxol-5 -yl)-1 H-pyrrolo[2,3 -b]pyridine-4 -yl)-1 H-indazol-3 -amine SU1330

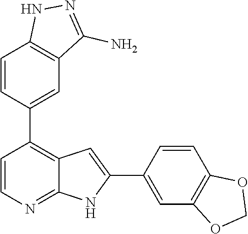 [SMILES: Nc1n[nH]c2ccc(-c3ccnc4[nH]c(-c5ccc6c(c5)OCO6)cc34)cc12]

To a suspension of 2 -(benzo[d][1,3 ]dioxol-5 -yl)-4 -chloro-1 H-pyrrolo[2,3 -b]pyridine (0.187 g, 0.69 mmol), 5 -(4,4,5,5 -tetramethyl-1,3,2 -dioxaborolan-2 -yl)-1 H-indazol-3 -amine (0.239 g, 0.92 mmol) and [1,1 ′-bis(di-tert-butylphosphino)ferrocene]dichloropalladium(II) catalyst (0.047 g, 0.072 mmol) in EtOH (3.0 mL, degassed under nitrogen) was added K₃ PO₄ (1 M, 1.2 mL) and the reaction mixture was heated to 100° C. for 20 h. The reaction mixture was cooled to room temperature and diluted with EtOAc, washed with water, aqueous saturated NaHCO₃ and brine. The organics were concentrated under reduced pressure and the resulting residue and purified by column chromatography (100 % EtOAc—10 % MeOH/EtOAc) and triturated with Et₂O to afford the title compound as a white solid (0.109 g, 0.30 mmol, 43 %). ¹H NMR (DMSO-d₆ ): δ 5.56 (br s, 2 H), 6.08 (br s, 2 H), 7.03 (d, J=8.0 Hz, 1 H), 7.11 (d, J=2.4 Hz, 1 H), 7.18 (d, J=4.8 Hz, 1 H), 7.39 (d, J=8.8 Hz, 1 H), 7.52 (dd, J=1.6, 8.0 Hz, 1 H), 7.59 (d, J=2.0 Hz, 1 H), 7.72 (dd, J=1.6, 8.4 Hz, 1 H), 8.23 (m, 2 H), 11.56 (br s, 2 H), 12.10 (br s, 2 H). HRMS: For C₂₁H₁₆O₂N₅ requires 370.1299 found 370.1293 (M+H)⁺..

### Route to SU1310

### 5 -(4 -Chloro-1 H-pyrrolo[2,3 -b]pyridin-2 -yl)-2 -fluorobenzonitrile

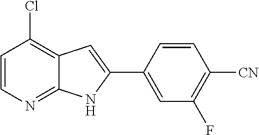 [SMILES: N#Cc1ccc(-c2cc3c(Cl)ccnc3[nH]2)cc1F]

A suspension of 4 -chloro-2 -iodo-7 -azaindole (0.32 g, 1.15 mmol), 4 -cyano-3 -fluorophenylboronic acid (0.219 g, 1.33 mmol), K₂ CO₃ (0.62 g, 4.49 mmol) and Bis(triphenylphosphine) palladium(II) chloride (0.053 g, 0.76 mmol) in 1,4 -dioxane (2 mL) and water (2 mL) was degassed under nitrogen. The reaction mixture was heated to 100° C. for 20 h. The reaction mixture was cooled to room temperature and the resulting precipitate was filtered under reduced pressure and washed with hexane to afford the title compound as an orange solid (0.185 g, 0.68 mmol, 59.3 %). ¹H NMR (DMSO-d₆ ): δ 7.06 (br s, 1 H), 7.21 (s, 1 H), 7.94 (t, J=6.0 Hz, 1 H), 8.03 (dd, J=1.2, 6.8 Hz, 1 H), 8.14 (m, 2 H).

### 5 -(4 -(3 -Amino-1 H-indazol-5 -yl)-1 H-pyrrolo[2,3 -b]pyridin-2 -yl)-2 -fluorobenzontrile SU1310

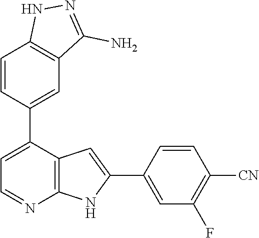 [SMILES: N#Cc1ccc(-c2cc3c(-c4ccc5[nH]nc(N)c5c4)ccnc3[nH]2)cc1F]

A suspension of 5 -(4 -chloro-1 H-pyrrolo[2,3 -b]pyridin-2 -yl)-2 -fluorobenzonitrile (0.11 g, 0.41 mmol), 5 -(4,4,5,5 -tetramethyl-1,3,2 -dioxaborolan-2 -yl)-1 H-indazol-3 -amine (0.181 g, 0.70 mmol) and [1,1 ′-bis(di-tert-butylphosphino)ferrocene]dichloropalladium(II) catalyst (0.065 g, 0.099 mmol) in EtOH (3.0 mL, degassed under nitrogen) was added K₃ PO₄ (1 M, 1.2 mL) and the reaction mixture was heated to 100° C. for 16 h. The reaction mixture was cooled to room temperature and the resulting precipitate was washed with hot MeOH (100 mL) and Et₂O (40 mL) to afford the title compound as an off-brown solid (0.152 g, 0.413 mmol, 100 %). ¹H NMR (DMSO-d₆ ): δ 5.55 (br s, 2 H), 7.26 (d, J=3.6 Hz, 1 H), 7.41 (d, J=7.2 Hz, 1 H), 7.55 (s, 1 H), 7.74 (d, J=6.8 Hz, 1 H), 8.03 (m, 2 H), 8.17 (d, J=9.2 Hz, 1 H), 8.25 (s, 1 H), 8.35 (d, J=4.0 Hz, 1 H), 11.58 (br s, 1 H), 12.48 (br s, 1 H). HRMS: For C₂₁H₁₄N₆ F requires 369.1258 found 369.1258 (M+H)⁺.

### 5 -(4 -(3 -Amino-1 H-indazol-5 -yl)-1 H-pyrrolo[2,3 b]pyridin-2 -yl)-2 -fluorobenzonitrile SU1301

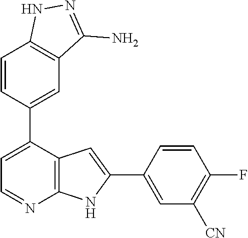 [SMILES: N#Cc1cc(-c2cc3c(-c4ccc5[nH]nc(N)c5c4)ccnc3[nH]2)ccc1F]

To a suspension of 5 -(4 -chloro-1 H-pyrrolo[2,3 -b]pyridin-2 -yl)-2 -fluorobenzonitrile (0.108 g, 0.40 mmol), 5 -(4,4,5,5 -tetramethyl-1,3,2 -dioxaborolan-2 -yl)-1 H-indazol-3 -amine (0.153 g, 0.59 mmol) and [1,1 ′-bis(di-tert-butylphosphino)ferrocene]dichloropalladium(II) catalyst (0.046 g, 0.071 mmol) in EtOH (3.0 mL, degassed under nitrogen) was added K₃ PO₄ (1 M, 1.2 mL) and the reaction mixture was heated to 80° C. for 24 h. The reaction mixture was cooled to room temperature and the reaction mixture was diluted with EtOAc, washed with water, aqueous saturated NaHCO₃ and brine. Organics were concentrated under reduced pressure and the resulting solid was washed with MeOH, hot MeOH and Et2O to afford the title compound with impurity (0.16 g, 0.44 mmol, 110 %). ¹H NMR (DMSO-d₆ ): δ 5.54 (br s, 2 H), 7.23 (d, J=4.0 Hz, 1 H), 7.38 (s, 1 H), 7.40 (d, J=6.8 Hz, 1 H), 7.67 (t, J=7.2 Hz, 1 H), 7.45 (d, J=8.0 Hz, 1 H), 8.23 (s, 1 H), 8.30 (d, J=4.0 Hz, 1 H), 8.39 (m, 1 H), 8.57 (m, 1 H), 11.57 (br s, 1 H), 12.33 (br s, 1 H).

### Route to SU1307

### 2 -(Pyrrolidin-1 -yl)-⁵ -(⁴,4,5,5 -tetramethyl-1,3,2 -dioxaborolan-2 -yl)benzonitrile

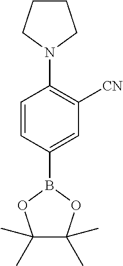 [SMILES: CC1(C)OB(c2ccc(N3CCCC3)c(C#N)c2)OC1(C)C]

2 -Fluoro-5 -(4,4,5,5 -tetramethyl-1,3,2 -dioxaborolan-2 -yl)benzonitrile (0.279 g, 1.13 mmol) was dissolved in 1,4 -dioxane (4 mL) to which pyrrolidine (0.44 mL, 5.27 mmol) was added. The reaction mixture was irradiated with microwaves at 140° C. for 10 mins. This process was repeated with a second batch as above. The reaction mixtures were combined, concentrated under reduced pressure and purified by column chromatography (100 % Hexane—9/1 Hexane/EtOAc) to afford the title compound as a white solid (0.3192 g, 1.07 mmol, 46.8 %).¹H NMR (DMSO-d₆ ): δ 1.27 (s, 12 H), 1.95 (m, 4 H), 3.55 (m, 4 H), 6.75 (d, J=8.0 Hz, 1 H), 7.59 (dd, J=8.0 Hz, 1 H), 7.65 (s, 1 H).

### 5 -(4 -Chloro-1 H-pyrrolo[2,3 -b]pyridin-2 -yl)-2 -(pyrrolindin-1 -yl)benzonitrile

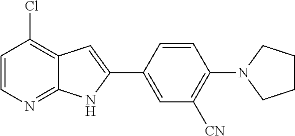 [SMILES: N#Cc1cc(-c2cc3c(Cl)ccnc3[nH]2)ccc1N1CCCC1]

A suspension of 4 -chloro-2 -iodo-7 -azaindole (0.251 g, 0.90 mmol), 2 -(pyrrolidin-1 -yl)-5 -(4,4,5,5 -tetramethyl-1,3,2 -dioxaborolan-2 -yl)benzonitrile (0.324 g, 1.09 mmol), K₂ CO₃ (0.45 g, 3.26 mmol) and Bis(triphenylphosphine) palladium(li) chloride (0.064 g, 0.09 mmol) in 1,4 -dioxane (2 mL) and water (2 mL) was degassed under nitrogen. The reaction mixture was heated to 100° C. for 20 h. The reaction mixture was cooled to room temperature and the resulting solid was filtered under reduced pressure, washed with water, hot MeOH and Et2O to afford the title compound as an off-white solid (0.26 g, 0.812 mmol, 74.7 %). ¹H NMR (DMSO-d₆ ): 5 1.97 (m, 4 H), 3.60 (m, 4 H), 6.86 (d, J=9.2 Hz, 1 H), 6.90 (d, J=2.0 Hz, 1 H), 7.16 (d, J=5.2 Hz, 1 H), 8.02 (dd, J=2.4, 9.2 Hz, 1 H), 8.11 (d, J=5.2 Hz, 1 H), 8.17 (d, J=2.0 Hz, 1 H), 12.32 (br s, 1 H).

### 5 -(4 -(3 -Amino-1 H-indazol-5 -yl)-1 H-pyrrolo[2,3 -b]pyridin-2 -yl)-2 -(pyrrolidin-1 -yl)benzonitrile SU1307

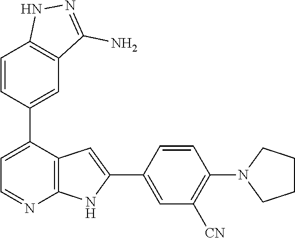 [SMILES: N#Cc1cc(-c2cc3c(-c4ccc5[nH]nc(N)c5c4)ccnc3[nH]2)ccc1N1CCCC1]

To a suspension of 5 -(4 -chloro-1 H-pyrrolo[2,3 -b]pyridin-2 -yl)-2 -(pyrrolindin-1 -yl)benzonitrile (0.109 g, 0.34 mmol), 5 -(4,4,5,5 -tetramethyl-1,3,2 -dioxaborolan-2 -yl)-1 H-indazol-3 -amine (0.146 g, 0.56 mmol) and [1,1 ′-bis(di-tert-butylphosphino)ferrocene]dichloropalladium(II) catalyst (0.049 g, 0.075 mmol) in EtOH (3.0 mL, degassed under nitrogen) was added K₃ PO₄ (1 M, 1.2 mL) and the reaction mixture was heated to 100° C. for 24 h. The reaction mixture was cooled to room temperature and the reaction mixture was diluted with EtOAc, washed with water, aqueous saturated NaHCO₃ and brine. Organics were concentrated under reduced pressure and the resulting solid was washed with water, MeOH, hot MeOH and Et₂O to afford the title compound as a brown solid (0.09 g, 0.21 mmol, 63.1 %). ¹H NMR (DMSO-d₆ ): δ 1.97 (m, 4 H), 3.59 (m, 4 H), 5.53 (br s, 2 H), 6.87 (d, J=7.2 Hz, 1 H), 7.11 (s, 1 H), 7.17 (d, J=3.6 Hz, 1 H), 7.39 (d, J=6.8 Hz, 1 H), 7.72 (d, J=7.6 Hz, 1 H), 8.02 (d, J=6.8 Hz, 1 H), 8.17 (s, 1 H), 8.20 (d, J=4.0 Hz, 1 H), 8.22 (s, 1 H), 11.54 (br s, 1 H), 12.06 (br s, 1 H). HRMS: For C₂₅H₂₂N₇ requires 420.1931 found 420.1932 . (M+H)⁺.

### Route to SU1329

### 5 -(4 -Chloro-1 H-pyrrolo[2,3 -b]pyridin-2 -yl)-2 -(methylpiperazin--yil)benzonitrile

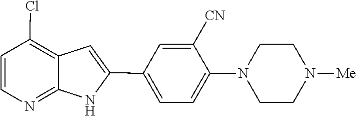 [SMILES: CN1CCN(c2ccc(-c3cc4c(Cl)ccnc4[nH]3)cc2C#N)CC1]

A suspension of 2 -(4 -methylpiperazin-1 -yl)-5 -(4,4,5,5 -tetramethyl-1,3,2 -dioxaborolan-2 -yl)benzonitrile (0.23 g, 0.73 mmol), 4 -chloro-2 -iodo-7 -azaindole (0.196 g, 0.79 mmol), K₂ CO₃ (0.412 g, 2.98 mmol) and Bis(triphenylphosphine) palladium(II) chloride (0.051 g, 0.073 mmol) in 1,4 -dioxane (3 mL) and water (2 mL) was degassed under nitrogen. The reaction mixture was heated to 100° C. for 20 h. The reaction mixture was cooled to room temperature and diluted with EtOAc, washed with water, aqueous saturated NaHCO₃, brine and concentrated under reduced pressure. The resulting solid was recrystallized from MeOH and hexane to afford the title compound as an off-white solid with some impurity present. This material was used crude at the next stage (0.157 g, 0.45 mmol, 50 %). ¹H NMR (DMSO-d₆ ): δ 2.24 (s, 3 H), 3.25 (m, 4 H), 7.05 (d, J=2.0 Hz, 1 H), 7.19 (d, J=4.4 Hz, 1 H), 7.23 (d, J=7.2 Hz, 1 H), 8.16 (d, J=4.0 Hz, 1 H), 8.18 (dd, J=2.0, 7.2 Hz, 1 H), 8.35 (d, J=1.6 Hz, 1 H), 12.46 (br s, 1 H).

### 5 -(4 -(3 -Amino-1 H-indazol-5 -yl)-1 H-pyrrolo[2,3 -b]pyridin-2 -yl)-2 -(4 -methylpiperazin-1 -yl)benzonitrile SU1329

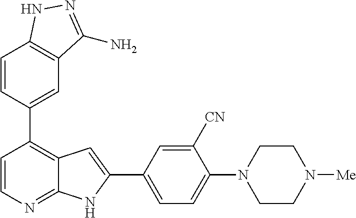 [SMILES: CN1CCN(c2ccc(-c3cc4c(-c5ccc6[nH]nc(N)c6c5)ccnc4[nH]3)cc2C#N)CC1]

To a suspension of 5 -(4 -chloro-1 H-pyrrolo[2,3 -b]pyridin-2 -yl)-2 -(methylpiperazin-1 -yl)benzonitrile (0.115 g, 0.33 mmol), 5 -(4,4,5,5 -tetramethyl-1,3,2 -dioxaborolan-2 -yl)-1 H-indazol-3 -amine (0.140 g, 0.54 mmol) and [1,1 ′-bis(di-tert-butylphosphino)ferrocene]dichloropalladium(II) catalyst (0.066 g, 0.10 mmol) in EtOH (3.0 mL, degassed under nitrogen) was added K₃ PO₄ (1 M, 1.2 mL) and the reaction mixture was heated to 80° C. for 20 h. The reaction mixture was cooled to room temperature and the resulting precipitate was filtered and washed with EtOAc and MeOH. The organics were combined, washed with water, aqueous saturated NaHCO₃ and brine. The organics were concentrated under reduced pressure and the resulting residue was partially dissolved in MeOH. To the filtrate was added hexane and the solvent concentrated to 1/3 total volume and the resulting precipitate was filter under reduced pressure and washed with Et₂O to afford the title compound as an off-white solid (0.040 g, 0.09 mmol, 27 %). ¹H NMR (DMSO-d₆ ): δ 2.55 (s, 3 H), 3.24 (m, 4 H), 5.53 (br s, 2 H), 7.20 (d, J=5.2 Hz, 1 H), 7.25 (q, J=2.9 Hz, 2 H), 7.40 (d, J=8.8 Hz, 1 H), 7.74 (dd, J=1.2, 8.4 Hz, 1 H), 8.19 (dd, J=2.4, 8.8 Hz, 1 H), 8.23 (s, 1 H), 8.26 (d, J=4.8 Hz, 1 H), 8.36 (d, J=2.0 Hz, 1 H), 11.56 (br s, 1 H), 12.19 (br s, 1 H). HRMS: For C₂₆H₂₆N₈ requires 449.2197 found 449.2195 . (M+H)⁺. 5,5 ′-(1 H-Pyrrolo[2,3 -b]pyridine-2,4 -diyl)bis(1 H-indazol-3 -amine) SU1302

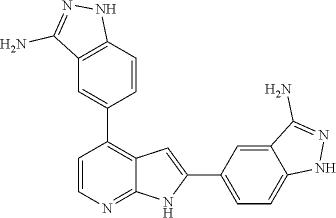 [SMILES: Nc1n[nH]c2ccc(-c3cc4c(-c5ccc6[nH]nc(N)c6c5)ccnc4[nH]3)cc12]

4 -Chloro-2 -iodo-1 H-pyrrolo[2,3 -b]pyridine (0.100 g, 0.23 mmol), 5 -(4,4,5,5 -tetramethyl-1,3,2 -dioxaborolan-2 -yl)-1 H-indazol-3 -amine (0.128 g, 0.35 mmol), [1,1 ′-bis(di-tert-butylphosphino)ferrocene]dichloropalladium(II) catalyst (0.020 mg, 0.017 mmol) and a magnetic stirrer bar were added in a 2-5 mL MV vial, and flushed with nitrogen. EtOH (4 mL) was added, and the resulting mixture was purged with nitrogen for 10 min before adding 1 M K₃ PO₄ (aqueous solution, 0.7 mL) and heated to 100° C. overnight. After cooling water was added and the solid precipitated was filtered and dried in vacuo. The crude was purified by flash chromatography (SiO₂, hexane:EtOAc 1:1-100 % EtOAc) to obtain the title compound as beige solid (0.016 g, 0.04 mmol, 17 %). ¹H NMR (500 MHz, DMSO-d₆ ) δ 6 7.08 (s, 1 H), 7.26 (d, J=5.2 Hz, 1 H), 7.42 (d, J=8.8 Hz, 1 H), 7.52 (d, J=8.8 Hz, 1 H), 7.84 (dd, J=5.2, 1.8 Hz, 1 H), 7.95 (dd, J=5.0, 1.8 Hz, 1 H), 8.29 (d, J=5.2 Hz, 2 H), 12.43 (s, 1 H). LRMS found 381.20 (M+H)⁺calculated for C₂₁H₁₆N₈ 380.15.

### 5 -(2 -(3,5 -Difluorophenyl)-1 H-pyrrolo[2,3 -b]pyridin-4 -yl)-1 H-indazol-3 -amine SU1372

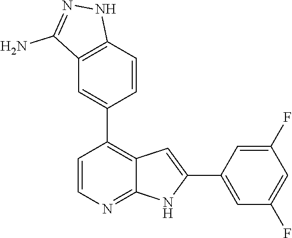 [SMILES: Nc1n[nH]c2ccc(-c3ccnc4[nH]c(-c5cc(F)cc(F)c5)cc34)cc12]

A 2-5 mL Biotage MW tube was charged with 4 -chloro-2 -(3 -fluoro-5 -methoxyphenyl)-1 H-pyrrolo[2,3 -b]pyridine (132 mg, 0.5 mmol, 1 eq.), 5 -(4,4,5,5 -tetramethyl-1,3,2 -dioxaborolan-2 -yl)-1 H-indazol-3 -amine (168 mg, 0.65 mmol, 1.3 eq.), [1,1 ′-bis(di-tert-butylphosphino)ferrocene]dichloropalladium(II) catalyst (16 mg, 0.025 mmol, 5 mol %) and 1,4 -dioxane (2.5 mL, oxygen free). The resulting suspension was purged with nitrogen for 5 minutes before adding 2 M aq. K₂ CO₃ (0.63 mL, oxygen free). The reaction mixture was heated to 50° C. under a gentle flow of nitrogen for 10 minutes, then to 110° C. for 24 hours. The stirred reaction mixture was then cooled to room temperature, slowly diluted with water (6 mL) and filtered. The filtered solid was then washed with water (3 mL×3 ) and hexane (3 mL×3 ).

Purification by flash chromatography gave the title compound as a yellow solid (70 mg, 39 %).

¹H NMR (400 MHz, DMSO-D₆ ) δ 5.56 (s, 2 H), 7.17-7.27 (m, 2 H), 7.37-7.44 (m, 2 H), 7.71 -7.76 (m, 1 H), 7.76-7.82 (m, 2 H), 8.23 (s, 1 H), 8.31 (d, J=4.9 Hz, 1 H), 11.58 (s, 1 H), 12.35 (s, 1 H). 5 -(2 -(2,3,5 -Trifluorophenyl)-1 H-pyrrolo[2,3 -b]pyridin-4 -yl)-1 H-indazol-3 -amine SU1392

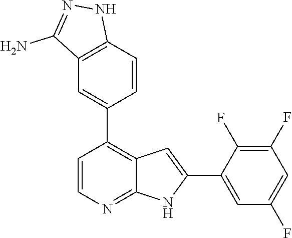 [SMILES: Nc1n[nH]c2ccc(-c3ccnc4[nH]c(-c5cc(F)cc(F)c5F)cc34)cc12]

A 2-5 mL MW tube was charged with 4 -chloro-2 -(2,3,5 -trifluorophenyl)-1 H-pyrrolo[2,3 -b]pyridine (75 mg, 0.265 mmol, 1 eq.), 5 -(4,4,5,5 -tetramethyl-1,3,2 -dioxaborolan-2 -yl)-1 H-indazol-3 -amine (89 mg, 0.345 mmol, 1.3 eq.), [1,1 ′-bis(di-tert-butylphosphino)ferrocene]dichloropalladium(li) catalyst (8.6 mg, 0.0133 mmol, 5 mol %) in 1,4 -dioxane (1.6 mL). The resulting suspension was stirred under nitrogen before adding 1 M aq. K₃ PO₄ (530 μL). The reaction mixture was heated to 50° C. under a gentle flow of nitrogen for 10 minutes, then to 110° C. for 19 hours. The stirred reaction mixture was then cooled to room temperature, slowly diluted with water (9 mL) and filtered. The filtered solid was then washed with water (3 mL×3 ) and hexane (3 mL×2 ) to give a crude, which was purified by flash chromatography (SiO₂, 50 g, 0-3 % MeOH in AcOEt) to afford the title compound as yellow solid (22 mg, 22 %). ¹H NMR (400 MHz, DMSO-D6) 6 5.55 (s, 2 H), 7.25 (d, J=5.0 Hz, 2 H), 7.41 (d, J=8.7 Hz, 1 H), 7.56 (s, 1 H), 7.69 (dd, J=8.7, 1.6 Hz, 1 H), 7.74-7.87 (m, 1 H), 8.20 (s, 1 H), 8.37 (d, J=4.9 Hz, 1 H), 11.59 (s, 1 H), 12.35 (s, 1 H).

### 5 -(2 -(Pyridin-2 -yl)-1 H-pyrrolo[2,3 -b]pyridin-4 -yl)-1 H-indazol-3 -amine SU1293

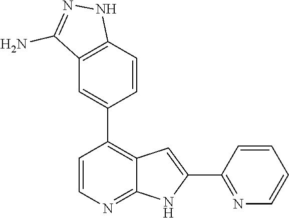 [SMILES: Nc1n[nH]c2ccc(-c3ccnc4[nH]c(-c5ccccn5)cc34)cc12]

A 2-5 mL MW vial was charged with of 4 -chloro-2 -iodo-1 H-pyrrolo[2,3 -b]pyridine (0.278 g, 1 mmol, 1 eq.) 2 -pyridylboronic acid MIDA ester (0.328 g, 1.4 mmol, 1.4 eq.), [1,1 ′-Bis(diphenylphosphino)ferrocene]dichloropalladium(II) catalyst (0.029 g, 0.004 mmol, 4 mol %), K₂ CO₃ (0.691 g, 5 mmol, 5 eq.), CuCl (0.099 g, 1 mmol, 1 eq.) in 4:1 DMF/i-propanol (5 mL).The resulting mixture was purged with N₂ at 50° C. for 10 minutes and then heated to 100° C. After 16 hours the reaction mixture was cooled to room temperature and diluted with EtOAc (5 mL), transferred to a 250 mL separating funnel with saturated aqueous NH₄ Cl (20 mL) and EtOAc (150 mL). The organic layer was separated and washed with water (50 mL×2 ), dried (MgSO₄ ) and filtered to give a grey solid (0.095 g). This intermediate was used in the next step without further purification.

A 2-5 mL MW vial was charged with 4 -chloro-2 -(pyridin-2 -yl)-1 H-pyrrolo[2,3 -b]pyridine (0.085 g, 0.370 mmol, 1 eq.) 5 -(4,4,5,5 -tetramethyl-1,3,2 -dioxaborolan-2 -yl)-1 H-indazol-3 -amine (0.115 g, 0.444 mmol, 1.2 eq.), [1,1 ′-bis(di-tert-butylphosphino)ferrocene]dichloropalladium(II) catalyst (0.0121 g, 0.0185 mmol, 5 mol %), sealed with an aluminium crimp cap fitted with a disposable PTFE/silicon septum, and purged with nitrogen. Ethanol (1.5 mL, O₂ -free) was added, and the mixture was purged with nitrogen at for 10 minutes before adding K₃ PO₄ (0.74 mL, 1 M in water, 02 -free). The mixture was heated to ca. 50° C. and purged with nitrogen for 10 minutes and then heated to 110° C. for 24 hours. The reaction mixture was then stirred at room temperature, diluted by dropwise addition of water (ca. 7 mL) and filtered to give a dark orange solid (0.124 g), which was purified by flash chromatography (Biotage SP4, 75 g SiO₄, 4 % MeOH in EtOAc) to afford the title compound as a yellow solid (0.0075 g, 0.023 mmol, 2 %). 1 H NMR (400 MHz, DMSO-d6 ) δ 5.56 (s, 2 H), 7.21 (d, J=4.9 Hz, 1 H), 7.31-7.36 (m, 1 H), 7.39 -7.44 (m, 2 H), 7.72 (d, J=8.9 Hz, 1 H), 7.87-7.92 (m, 2 H), 8.11 (d, J=8.0 Hz, 1 H), 8.24 (s, 1 H), 8.31 (d, J=5.0 Hz, 1 H), 8.66 (d, J=4.8 Hz, 1 H), 11.58 (s, 1 H), 12.30 (s, 1 H). HRMS: Calculated for C₁₉H₁₅N₆ (M+H*)=327.1353 ; Found: 327.1354 5 -(2 -(pyridin-3 -yl)-1 H-pyrrolo[2,3 -b]pyridin-4 -yl)-1 H-indazol-3 -amine SU1322

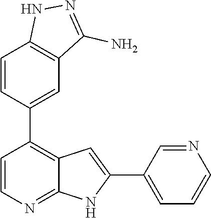 [SMILES: Nc1n[nH]c2ccc(-c3ccnc4[nH]c(-c5cccnc5)cc34)cc12]

A suspension of 4 -chloro-2 -(pyridin-3 -yl)-1 H-pyrrolo[2,3 -b]pyridine (0.237 g, 1.036 mmol), 5 -(4,4,5,5 -tetramethyl-1,3,2 -dioxaborolan-2 -yl)-1 H-indazol-3 -amine (0.375 g, 1.45 mmol), [1,1 ′-bis(di-tert-butylphosphino)ferrocene]dichloropalladium(II) catalyst (0.034 g, 0.05 mmol) and a magnetic stirrer bar were added in a 10-20 mL Biotage microwave vial which was sealed with an aluminium crimp cap fitted with a disposable PTFE/silicon septum, and flushed with nitrogen. Then EtOH (4 mL) was added, and the resulting mixture was purged with nitrogen for 10 min before adding 1 M K₃ PO₄ (aqueous solution, 2 mL). The mixture was purged again with nitrogen for 10 min and heated to 100° C. overnight. After cooling water was added to the reaction mixture and the solid that precipitated was filtered and dried in vacuo. The crude was purified by flash chromatography (SiO₂, hexane:EtOAc 40:60 - hexane:EtOAc 20:80 ) to obtain the title compound as brown solid (0.030 g, 0.092 mmol, 9 %). ¹H NMR (400 MHz, DMSO-d₆ ) δ 5.57 (s, 2 H), 7.23 (d, J=5.0 Hz, 1 H), 7.35 (d, J=1.9 Hz, 1 H), 7.40 (d, J=8.5 Hz, 1 H), 7.50 (dd, J=7.9, 4.8 Hz, 1 H), 7.74 (d, J=8.5 Hz, 1 H), 8.25 (s, 1 H), 8.30 (d, J=5.0 Hz, 1 H), 8.35 (d, J=7.9 Hz, 1 H), 8.54 (d, J=4.8 Hz 1 H), 9.22 (s, 1 H), 11.58 (s, 1 H), 12.39 (s, 1 H). HRMS found 327.13 (M+H)⁺calculated for C₉H₁₅N₅ 327.14 5 -(2 -(Pyridin-4 -yl)-1 H-pyrrolo[2,3 -b]pyridin-4 -yl)-1 H-indazol-3 -amine SU1331

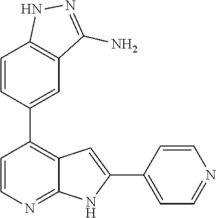 [SMILES: Nc1n[nH]c2ccc(-c3ccnc4[nH]c(-c5ccncc5)cc34)cc12]

4 -Chloro-2 -(pyridin-4 -yl)-1 H-pyrrolo[2,3 -b]pyridine (0.093 g, 0.28 mmol), 5 -(4,4,5,5 -tetramethyl-1,3,2 -dioxaborolan-2 -yl)-1 H-indazol-3 -amine (0.087 g, 0.33 mmol), [1,1 ′-bis(di-tert-butylphosphino)ferrocene]dichloropalladium(II) catalyst (0.009 g, 0.014 mmol) and a magnetic stirrer bar were added in a 2-5 mL Biotage microwave vial which was sealed with an aluminium crimp cap fitted with a disposable PTFE/silicon septum, and flushed with nitrogen. Then EtOH (3 mL) was added, and the resulting mixture was purged with nitrogen for 10 min before adding 1 M K₃ PO₄ (aqueous solution, 0.5 mL). The mixture was purged again with nitrogen for 10 min and heated to 100° C. for 24 hours. After cooling water was added to the reaction mixture and the solid that precipitated was filtered and dried in vacuo. The crude was purified by flash chromatography (SiO₂, EtOAc—EtOAc:MeOH 90:10 ) to obtain the title compound as brown solid (0.021 g, 0.05 mmol, 16 %). ¹H NMR (400 MHz, DMSO-d₆) 6 5.58 (s, 2 H), 7.25 (d, J=4.8 Hz, 1 H), 7.41 (d, J=8.0 Hz, 1 H), 7.49 (s, 1 H), 7.73 (d, J=8.0 Hz, 1 H), 7.97 (d, J=4.2 Hz, 2 H), 8.25 (s, 1 H), 8.34 (d, J=4.8 Hz, 1 H), 8.64 (d, J=4.2 Hz, 2 H), 11.60 (s, 1 H), 12.51 (s, 1 H).

LRMS found 183.13 (M+H)⁺⁺ calculated for C₁₂H₁₁N₂ 183.09 5 -(2 -(Pyrimidin-5 -yl)-1 H-pyrrolo[2,3 -b]pyridin-4 -yl)-1 H-indazol-3 -amine SU1347

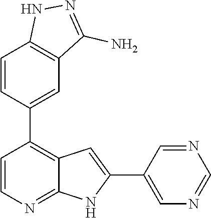 [SMILES: Nc1n[nH]c2ccc(-c3ccnc4[nH]c(-c5cncnc5)cc34)cc12]

4 -Chloro-2 -(pyrimidin-5 -yl)-1 H-pyrrolo[2,3 -b]pyridine (0.045 g, 0.20 mmol), 5 -(4,4,5,5 -tetramethyl-1,3,2 -dioxaborolan-2 -yl)-1 H-indazol-3 -amine (0.061 g, 0.23 mmol), [1, 1 ′-bis(di-tert-butylphosphino)ferrocene]dichloropalladium(li) catalyst (0.006 g, 0.098 mmol) and a magnetic stirrer bar were added in a 2-5 mL Biotage microwave vial which was sealed with an aluminium crimp cap fitted with a disposable PTFE/silicon septum, and flushed with nitrogen. Then EtOH (2 mL) was added, and the resulting mixture was purged with nitrogen for 10 min before adding 1 M K₃ PO₄ (aqueous solution, 0.4 mL). The mixture was purged again with nitrogen for 10 min and heated to 100° C. for 24 hours. After cooling water was added to the reaction mixture and the solid that precipitated was filtered and dried in vacuo. The crude was purified by flash chromatography (SiO₂, dichloromethane—dichloromethane: MeOH 85:15 ) to obtain the title compound as beige solid (0.018 g, 0.06 mmol, 27 %). ¹H NMR (400 MHz, DMSO-d₆ ) δ 5.56 (s, 2 H), 7.26 (d, J=5.0 Hz, 1 H), 7.41 (d, J=8.7 Hz, 1 H), 7.50 (s, 1 H), 7.74 (d, J=8.7 Hz, 1 H), 8.24 (s, 1 H), 8.34 (d, J=5.0 Hz, 1 H), 9.14 (s, 1 H), 9.41 (s, 2 H), 11.59 (s, 1 H), 12.52 (s, 1 H). HRMS (ESI) found 314.1401 (M+H)⁺calculated for C₁₉H₁₆N₅ 314.1400.

### 4-(4-(3-Amino-1H-indazol-5-yl)-1H-pyrrolo[2,3-b]pyridin-2-yl)pyridin-2(1H)-one SU1610

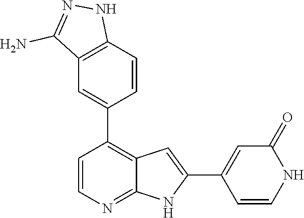 [SMILES: Nc1n[nH]c2ccc(-c3ccnc4[nH]c(-c5cc[nH]c(=O)c5)cc34)cc12]

To a stirred suspension of 5 -(2 -(2 -fluoropyridin-4 -yl)-1 H-pyrrolo[2,3 -b]pyridin-4 -yl)-1 H-indazol-3 -amine (SU1339 ) (69 mg, 0.2 mmol, 1 eq.) in 1,4 -dioxane (1.5 mL) was added 1 M aq. HCl (1 mL). The reaction mixture was sealed under an argon atmosphere and heated to 110° C. for 4 hours under microwave irradiation. The reaction mixture was concentrated in vacuo and the residue was suspended in AcOEt (20 mL), washed with sat. aq. NaHCO₃ (20 mL), dried (MgSO₄ ) and concentrated in vacuo to give the title compound as a yellow solid (64 mg). ¹H NMR (400 MHz, DMSO) δ 5.54 (s, 2 H), 6.75-6.81 (m, 1 H), 6.94 (s, 1 H), 7.18-7.24 (m, 1 H), 7.32 (s, 1 H), 7.36-7.44 (m, 2 H), 7.72 (dd, J=8.7, 1.7 Hz, 1 H), 8.24 (s, 1 H), 8.31 (d, J=4.8 Hz, 1 H). NH peaks not observed. 5 -(2 -(2 -Fluoropyridin-4 -yl)-1 H-pyrrolo[2,3 -b]pyridin-4 -yl)-1 H-indazol-3 -amine SU1339

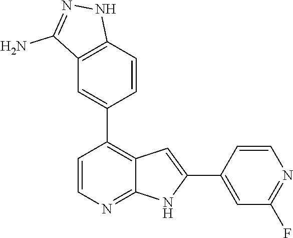 [SMILES: Nc1n[nH]c2ccc(-c3ccnc4[nH]c(-c5ccnc(F)c5)cc34)cc12]

A 2-5 mL microwave vial was charged with 4 -chloro-2 -(2 -fluoropyridin-4 -yl)-1 H-pyrrolo[2,3 -b]pyridine (0.110 g, 0.444 mmol, 1 eq), 5 -(4,4,5,5 -tetramethyl-1,3,2 -dioxaborolan-2 -yl)-1 H-indazol-3 -amine (0.150 g, 0.577 mmol, 1.3 eq.), [1,1 ′-bis(di-tert-butylphosphino)ferrocene]dichloropalladium(II) catalyst (0.0145 g, 5 mol %), sealed with an aluminium crimp cap fitted with a disposable PTFE/silicon septum, and purged with nitrogen.

Ethanol (1.8 mL) was added, and the mixture was stirred under nitrogen for 10 minutes before adding K₃ PO₄ (0.89 mL, 1 M in water, O₂ -free). The mixture was heated to ca. 50° C. and purged with nitrogen for 10 minutes. The resulting mixture was then heated to 110° C. for 26 hours.

The reaction mixture was then cooled to room temperature and diluted with water (ca. 7 mL) to give a precipitate, which was filtered and purified by flash chromatography (Biotage SP4, 50 g SiO₄, 5-6 % and then 20-25 % MeOH in EtOAc) to afford 5 -(2 -(2 -fluoropyridin-4 -yl)-1 H-pyrrolo[2,3 -b]pyridin-4 -yl)-1 H-indazol-3 -amine as a yellow solid (0.042 g, 0.122 mmol, 27 %) 1 H NMR (400 MHz, DMSO-d6 ) δ 2.29 (s, 3 H), 5.47 (s, 2 H), 5.83 (s, 2 H), 6.50 (s, 1 H), 6.66 (s, 1 H), 7.28 (d, J=8.7 Hz, 1 H), 7.49 (d, J=8.7 Hz, 1 H), 8.04 (s, 1 H), 11.49 (s, 1 H) 5 -(2 -(2 -Fluoro-6 -methylpyridin-4 -yl)-1 H-pyrrolo[2,3 -b]pyridin-4 -yl)-1 H-indazol-3 -amine SU1378

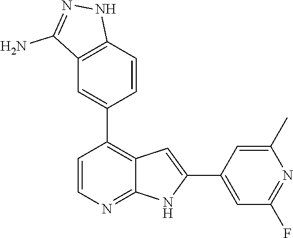 [SMILES: Cc1cc(-c2cc3c(-c4ccc5[nH]nc(N)c5c4)ccnc3[nH]2)cc(F)n1]

4 -Chloro-2 -(2 -fluoro-6 -methylpyridin-4 -yl)-1 H-pyrrolo[2,3 -b]pyridine (0.1 g, 0.4 mmol), 5 -(4,4,5,5 -tetramethyl-1,3,2 -dioxaborolan-2 -yl)-1 H-indazol-3 -amine (0.155 g, 0.6 mmol), [1,1 ′-bis(di-tert-butylphosphino)ferrocene]dichloropalladium(II) catalyst (0.02 mg, 0.017 mmol) and a magnetic stirrer bar were added in a 2-5 mL MV vial, and flushed with nitrogen. EtOH (4 mL) was added, and the resulting mixture was purged with nitrogen for 10 min before adding 1 M K₃ PO₄ (aqueous solution, 0.7 mL) and heated to 100° C. overnight. After cooling water was added and the solid precipitated was filtered and dried in vacuo. The crude was purified by flash chromatography (SiO₂, hexane:EtOAc 1:1-100 % EtOAc) to obtain the title compound as beige solid (0.037 g, 0.10 mmol, 25 %). ¹H NMR (500 MHz, DMSO-d₆ ) δ 2.48 (s, 3 H), 5.54 (s, 2 H), 7.27 (d, J=1.8 Hz, 1 H), 7.49 (d, J=10 Hz, 1 H), 7.55 (m, 2 H), 7.85 (m, 3 H), 8.31 (s, 1 H), 8.37 (d, J=7.5 Hz, 1 H), 12.53 (s, 1 H). LRMS found 359.20 (M+H)⁺calculated for C₂₀H₁₅FNs 358.13.

### 5 -(2 -(2,6 -Difluoropyridin-4 -yl)-1 H-pyrrolo[2,3 -b]pyridin-4 -yl)-1 H-indazol-3 -amine SU1361

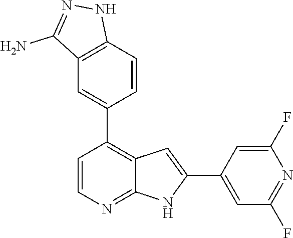 [SMILES: Nc1n[nH]c2ccc(-c3ccnc4[nH]c(-c5cc(F)nc(F)c5)cc34)cc12]

A 2-5 mL MW tube was charged with 4 -chloro-2 -(2,6 -difluoropyridin-4 -yl)-1 H-pyrrolo[2,3 -b]pyridine (213 mg, 0.8 mmol, 1 eq.), 5 -(4,4,5,5 -tetramethyl-1,3,2 -dioxaborolan-2 -yl)-1 H-indazol-3 -amine (269 mg, 1.04 mmol, 1.3 eq.), [1,1 ′-bis(di-tert-butylphosphino)ferrocene]dichloropalladium(II) catalyst (42 mg, 8 mol %) in 1,4 -dioxane (4 mL). The resulting suspension was stirred under nitrogen before adding 2 M aq. K₂ CO₃ (1 mL, 2.5 eq.). The reaction mixture was heated to 50° C. under a gentle flow of nitrogen for 10 minutes, then to 100° C. for 24 hours. The stirred reaction mixture was then cooled to room temperature, slowly diluted with water (40 mL) and filtered. The filtered solid was then washed with water (5 mL×2 ) and hexane (10 mL×3 ) to give a crude (390 mg), which was purified by flash chromatography (SiO₂, 75 g, 2 % MeOH in AcOEt) to afford the title compound as a yellow solid (34 mg, 12 %). ¹H NMR (400 MHz, DMSO-D₆ ) δ 5.57 (s, 2 H), 7.29 (d, J=4.7 Hz, 1 H), 7.41 (d, J=8.6 Hz, 1 H), 7.69-7.77 (m, 2 H), 7.77-7.83 (m, 2 H), 8.24 (s, 1 H), 8.40 (d, J=4.6 Hz, 1 H), 11.61 (s, 1 H), 12.60 (s, 1 H).

### 5 -(2 -(2 -(Piperazin-1 -yl)pyridin-4 -yl)-1 H-pyrrolo[2,3 -b]pyridin-4 -yl)-1 H-indazol-3 -amine SU1617

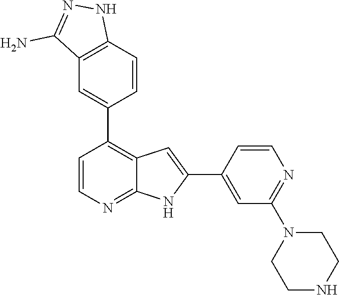 [SMILES: Nc1n[nH]c2ccc(-c3ccnc4[nH]c(-c5ccnc(N6CCNCC6)c5)cc34)cc12]

A 0.2-0.5 mL Biotage tube was charged with 5 -(2 -(2 -fluoropyridin-4 -yl)-1 H-pyrrolo[2,3 -b]pyridin-4 -yl)-1 H-indazol-3 -amine (SU1339 ) (20.7 mg, 0.06 mmol, 1 eq.) and piperazine (100 μL) in anhydrous DMSO (400 μL) and sealed under an argon atmosphere. The reaction mixture was heated to 210° C. under microwave irradiation for 20 minutes and then purified by HPLC to give the title compound as a yellow solid. ¹H NMR (400 MHz, DMSO) δ 3.18-3.29 (m, 4 H), 3.79 -3.86 (m, 4 H), 7.25 (d, J=5.0 Hz, 1 H), 7.37 (dd, J=5.4, 1.3 Hz, 1 H), 7.42-7.49 (m, 2 H), 7.51 (s, 1 H), 7.78 (dd, J=8.7, 1.7 Hz, 1 H), 8.21 (d, J=5.4 Hz, 1 H), 8.27 (d, J=1.6 Hz, 1 H), 8.35 (d, J=5.0 Hz, 1 H), 8.80 (bs, 2 H), 11.84 (s, 1 H), 12.41 (s, 1 H). NH₂ peak not observed.

### 5 -(2 -(2 -Morpholinopyridin-4 -yl)-1 H-pyrrolo[2,3-b]pyridin-4 -yl)-1 H-indazol-3 -amine SU1618

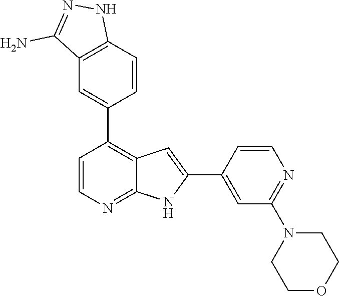 [SMILES: Nc1n[nH]c2ccc(-c3ccnc4[nH]c(-c5ccnc(N6CCOCC6)c5)cc34)cc12]

A 0.2-0.5 mL Biotage tube was charged with 5 -(2 -(2 -fluoropyridin-4 -yl)-1 H-pyrrolo[2,3 -b]pyridin-4 -yl)-1 H-indazol-3 -amine (SU1339 ) (20.7 mg, 0.06 mmol, 1 eq.) and morpholine (100 μL) in anhydrous DMSO (400 μL) and sealed under an argon atmosphere. The reaction mixture was heated to 210° C. under microwave irradiation for 20 minutes and then purified by HPLC to give the title compound as a yellow solid. ¹H NMR (400 MHz, DMSO) δ 3.59-3.67 (m, 4 H), 3.74 -3.81 (m, 4 H), 7.27 (d, J=5.0 Hz, 1 H), 7.42 (d, J=5.9 Hz, 1 H), 7.47 (d, J=8.7 Hz, 1 H), 7.53 -7.62 (m, 2 H), 7.80 (dd, J=8.7, 1.7 Hz, 1 H), 8.14 (d, J=5.9 Hz, 1 H), 8.30 (d, J=1.5 Hz, 1 H), 8.39 (d, J=5.0 Hz, 1 H), 11.75-12.11 (bs, 1 H), 12.53 (s, 1 H). NH₂ peak not observed. 5 -(2 -(2 -(4 -(tert-Butyl)piperazin-1 -yl)pyridin-4 -yl)-1 H-pyrrolo[2,3 -b]pyridin-4 -yl)-1 H-indazol-3 -amine SU1619

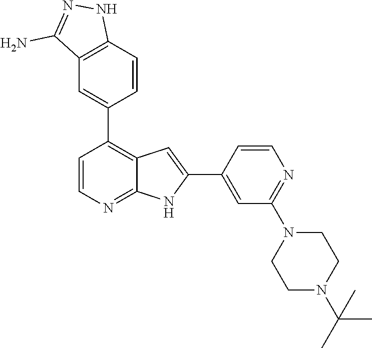 [SMILES: CC(C)(C)N1CCN(c2cc(-c3cc4c(-c5ccc6[nH]nc(N)c6c5)ccnc4[nH]3)ccn2)CC1]

A 0.2-0.5 mL Biotage tube was charged with 5 -(2 -(2 -fluoropyridin-4 -yl)-1 H-pyrrolo[2,3 -b]pyridin-4 -yl)-1 H-indazol-3 -amine (SU1339 ) (20.7 mg, 0.06 mmol, 1 eq.), N-(tert-butyl)piperazine (100 μL) and anhydrous DMSO (400 μL) and sealed under an argon atmosphere. The reaction mixture was heated to 210° C. under microwave irradiation for 20 minutes and then purified by HPLC to give the title compound as a yellow solid. ¹H NMR (400 MHz, DMSO) δ 1.38 (s, 9 H), 3.05-3.27 (m, 4 H), 3.66 (d, J=11.3 Hz, 2 H), 4.59 (d, J=13.2 Hz, 2 H), 7.25 (d, J=5.0 Hz, 1 H), 7.38 (dd, J=5.3, 1.3 Hz, 1 H), 7.42-7.49 (m, 2 H), 7.54 (s, 1 H), 7.77 (dd, J=8.7, 1.7 Hz, 1 H), 8.22 (d, J=5.4 Hz, 1 H), 8.25-8.29 (m, 1 H), 8.35 (d, J=5.0 Hz, 1 H), 9.20-9.35 (m, 1 H), 11.73-11.92 (bs, 1 H), 12.43 (s, 1 H). NH₂ peak not observed. 4 -(4 -(4 -(3 -Amino-1 H-indazol-5 -yl)-1 H-pyrrolo[2,3 -b]pyridin-2 -yl)pyridin-2 -yl)thiomorpholine 1,1 -dioxide SU1629

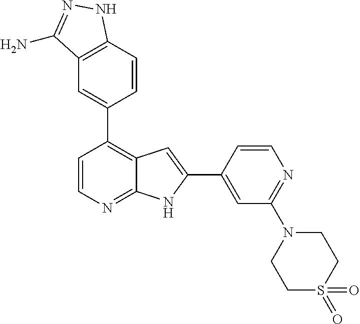 [SMILES: Nc1n[nH]c2ccc(-c3ccnc4[nH]c(-c5ccnc(N6CCS(=O)(=O)CC6)c5)cc34)cc12]

A 0.2-0.5 mL Biotage tube was charged with 5 -(2 -(2 -fluoropyridin-4 -yl)-1 H-pyrrolo[2,3 -b]pyridin-4 -yl)-1 H-indazol-3 -amine (SU1339 ) (20.7 mg, 0.06 mmol, 1 eq.), thiomorpholine 1,1 -dioxide (100 μL) and anhydrous 1,3 -dimethyl-2 -imidazolidinone (400 μL) and sealed under an argon atmosphere. The reaction mixture was heated to 210° C. under microwave irradiation for 20 minutes and then purified by HPLC to give the title compound as a yellow solid. ¹H NMR (500 MHz, DMSO) δ 3.15-3.22 (m, 4 H), 4.12-4.20 (m, 4 H), 7.25 (d, J=5.0 Hz, 1 H), 7.36 (d, J=5.4 Hz, 1 H), 7.42-7.50 (m, 2 H), 7.58 (s, 1 H), 7.80 (dd, J=8.6, 1.7 Hz, 1 H), 8.21 (d, J=5.4 Hz, 1 H), 8.29 (s, 1 H), 8.36 (d, J=5.0 Hz, 1 H), 11.76-12.03 (bs, 1 H), 12.42 (s, 1 H). NH₂ peak not observed. 5 -(2 -(2,6 -Dimorpholinopyridin-4 -yl)-1 H-pyrrolo[2,3 -b]pyridin-4 -yl)-1 H-indazol-3 -amine SU1630

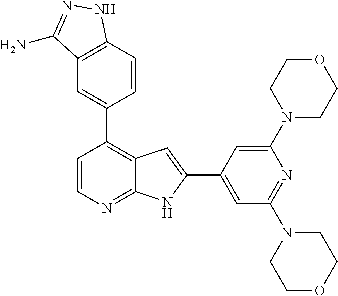 [SMILES: Nc1n[nH]c2ccc(-c3ccnc4[nH]c(-c5cc(N6CCOCC6)nc(N6CCOCC6)c5)cc34)cc12]

A 0.5-2 mL Biotage tube was charged with 5 -(2 -(2,6 -difluoropyridin-4 -yl)-1 H-pyrrolo[2,3 -b]pyridin-4 -yl)-1 H-indazol-3 -amine (SU1361 ) (29 mg, 0.08 mmol, 1 eq.), phenylmethanethiol (10.9 mg, 10.3 μL, 0.088 mmol, 1.1 eq.) sodium methoxide (4.75 mg, 0.088 mmol, 1.1 eq.) and anhydrous 1,3 -dimethyl-2 -imidazolidinone (1 mL) and sealed under an argon atmosphere. The reaction mixture was heated to 90° C. for 19 hours and then cooled to room temperature, diluted with morpholine (250 μL) and heated to 210° C. under argon for 20 minutes under microwave irradiation. The reaction mixture was purified by HPLC to give the title compound as a side product. ¹H NMR (500 MHz, DMSO) δ 3.46-3.51 (m, 8 H), 3.69-3.74 (m, 8 H), 6.75 (s, 2 H), 7.22 (d, J=5.0 Hz, 1 H), 7.36 (d, J=2.1 Hz, 1 H), 7.49 (d, J=8.7 Hz, 1 H), 7.81 (dd, J=8.7, 1.7 Hz, 1 H), 8.29 (s, 1 H), 8.32 (d, J=5.0 Hz, 1 H), 11.83-12.15 (bs, 1 H), 12.28 (s, 1 H). NH2 peak not observed.

### 4 -(4 -(3 -Amino-1 H-indazol-5 -yl)-1 H-pyrrolo[2,3 -b]pyridin-2 -yl)-1 -(4 -(piperidin-1 -ylmethyl)benzyl)pyridin-2 (1 H)-one SU1611

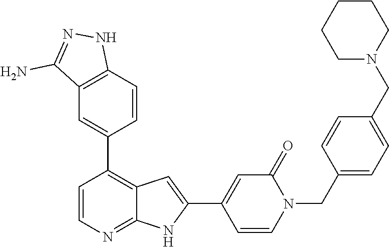 [SMILES: Nc1n[nH]c2ccc(-c3ccnc4[nH]c(-c5ccn(Cc6ccc(CN7CCCCC7)cc6)c(=O)c5)cc34)cc12]

A stirred solution of 4 -(4 -(3 -amino-1 H-indazol-5 -yl)-1 H-pyrrolo[2,3 -b]pyridin-2 -yl)pyridin-2 (1 H)-one (SU1610 ) (20.5 mg, 0.06 mmol,1 eq.) in DMSO (1 mL) was added to a vial containing potassium tert-butoxide (6.72 mg). To the resulting mixture was added 1,4 -bis(chloromethyl)benzene (42 mg, 0.24 mmol, 4 eq.). The resulting dark brown solution was stirred at room temperature for 1.5 hours. Then piperidine (189.7 μL 1.92 mmol, 32 eq.) was added and the reaction mixture was stirred at room temperature for 16 hours. Purification by HPLC gave the title compound as a yellow solid (13.4 mg). ¹H NMR (400 MHz, DMSO) δ 1.25 -1.43 (m, 1 H), 1.48-1.71 (m, 3 H), 1.76-1.84 (m, 2 H), 2.76-2.92 (m, 2 H), 3.26-3.35 (m, 2 H), 4.25 (d, J=5.3 Hz, 2 H), 5.16 (s, 2 H), 6.92 (dd, J=7.2, 2.1 Hz, 1 H), 7.09 (d, J=2.1 Hz, 1 H), 7.26 (d, J=5.0 Hz, 1 H), 7.38-7.51 (m, 6 H), 7.77 (dd, J=8.8, 1.7 Hz, 1 H), 7.89 (d, J=7.2 Hz, 1 H), 8.26 (d, J=1.7 Hz, 1 H), 8.36 (d, J=4.9 Hz, 1 H), 9.22 (bs, 1 H), 11.79 (s, 1 H), 12.37 (s, 1 H). NH₂ peak not observed.

### 4 -(4 -(3 -Amino-1 H-indazol-5 -yl)-1 H-pyrrolo[2,3 -b]pyridin-2 -yl)-1 -(3 -(piperidin-1 -ylmethyl)benzyl)pyridin-2 (1 H)-one SU1612

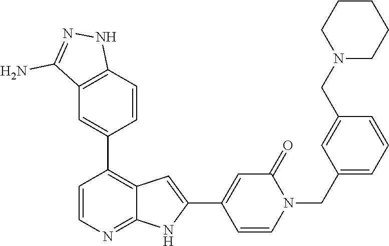 [SMILES: Nc1n[nH]c2ccc(-c3ccnc4[nH]c(-c5ccn(Cc6cccc(CN7CCCCC7)c6)c(=O)c5)cc34)cc12]

A stirred solution of 4 -(4 -(3 -amino-1 H-indazol-5 -yl)-1 H-pyrrolo[2,3 -b]pyridin-2 -yl)pyridin-2 (1 H)-one (SU1610 ) (17.1 mg, 0.05 mmol, 1 eq.) in DMSO (1 mL) was added to a vial containing potassium tert-butoxide (5.61 mg). To the resulting mixture was added 1,3 -bis(chloromethyl)benzene (48 mg, 0.25 mmol, 5 eq.). The resulting dark brown solution was stirred at room temperature for 1.5 hours. Then piperidine (123.4 μL, 1.25 mmol, 25 eq.) was added and the reaction mixture was stirred at room temperature for 16 hours. Purification by HPLC gave the title compound as a yellow solid. ¹H NMR (400 MHz, DMSO) δ 1.27-1.44 (m, 1 H), 1.49-1.72 (m, 3 H), 1.73-1.86 (m, 2 H), 2.80-2.94 (m, 2 H), 3.24-3.36 (m, 2 H), 4.26 (d, J=5.2 Hz, 2 H), 5.16 (s, 2 H), 6.92 (dd, J=7.3, 2.0 Hz, 1 H), 7.09 (d, J=2.0 Hz, 1 H), 7.25 (d, J=4.9 Hz, 1 H), 7.35-7.52 (m, 6 H), 7.77 (dd, J=8.6, 1.7 Hz, 1 H), 7.86 (d, J=7.2 Hz, 1 H), 8.27 (d, J=1.6 Hz, 1 H), 8.35 (d, J=5.0 Hz, 1 H), 9.30 (bs, 1 H), 11.85 (s, 1 H), 12.38 (s, 1 H). NH₂ peak not observed.

### 4 -(4 -(3 -Amino-1 H-indazol-5 -yl)-1 H-pyrrolo[2,3 -b]pyridin-2 -yl)-1 -(2 -(piperidin-1 -ylmethyl)benzyl)pyridin-2 (1 H)-one SU1613

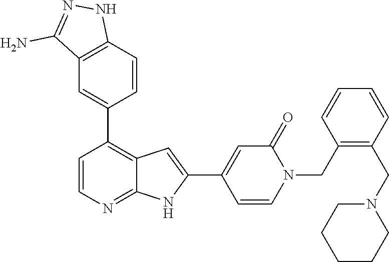 [SMILES: Nc1n[nH]c2ccc(-c3ccnc4[nH]c(-c5ccn(Cc6ccccc6CN6CCCCC6)c(=O)c5)cc34)cc12]

A stirred solution of 4 -(4 -(3 -amino-1 H-indazol-5 -yl)-1 H-pyrrolo[2,3 -b]pyridin-2 -yl)pyridin-2 (1 H)-one (SU1610 ) (17.1 mg, 0.05 mmol, 1 eq.) in DMSO (1 mL) was added to vial containing potassium tert-butoxide (5.61 mg). To the resulting mixture was added 1,2 -bis(chloromethyl)benzene (48 mg, 0.25 mmol, 5 eq.). The resulting dark brown solution was stirred at room temperature for 1.5 hours. Then piperidine (123.4 μL, 1.25 mmol, 25 eq.) was added and the reaction mixture was stirred at room temperature for 16 hours. Purification by HPLC gave the title compound as a yellow solid. ¹H NMR (400 MHz, DMSO) δ 1.36-1.53 (m, 1 H), 1.66-1.83 (m, 3 H), 1.86-1.94 (m, 2 H), 2.99-3.11 (m, 2 H), 3.47 (d, J=11.8 Hz, 2 H), 4.54 (d, J=5.1 Hz, 2 H), 5.29 (s, 2 H), 7.19 (d, J=2.0 Hz, 1 H), 7.22 (dd, J=7.1, 2.0 Hz, 1 H), 7.27 (d, J=5.0 Hz, 1 H), 7.28-7.33 (m, 1 H), 7.37-7.54 (m, 5 H), 7.77 (dd, J=8.7, 1.7 Hz, 1 H), 8.26 (d, J=1.6 Hz, 1 H), 8.30 (d, J=7.2 Hz, 1 H), 8.37 (d, J=5.0 Hz, 1 H), 9.92-10.07 (bs, 1 H), 11.67-11.89 (bs, 1 H), 12.37 (s, 1 H). NH₂ peak not observed.

### Route to SU1342

### 4 -Chloro-2 -(5 -methoxypyridin-3 -yl)-1 H-pyrolo[2,3 -b]pyridine

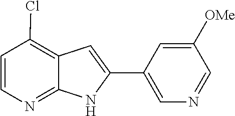 [SMILES: COc1cncc(-c2cc3c(Cl)ccnc3[nH]2)c1]

A suspension of 4 -chloro-2 -iodo-7 -azaindole (0.316 g, 1.13 mmol), 3 -methoxy-5 -(4,4,5,5 -tetramethyl-1,3,2 -dioxaborolan-2 -yl)pyridine (0.33 g, 1.40 mmol), K₂ CO₃ (0.63 g, 4.56 mmol) and Bis(triphenylphosphine) palladium(II) chloride (0.075 g, 0.107 mmol) in 1,4 -dioxane (3 mL) and water (2 mL) was degassed under nitrogen. The reaction mixture was heated to 100° C. for 20 h. The reaction mixture was cooled to room temperature and extracted into EtOAc, washed with water, aqueous saturated NaHCO₃ and brine. Organics were concentrated under reduced pressure and purified by column chromatography (100 % Hexane—1/1 Hexane/EtOAc—100 % EtOAc—10 % MeOH/EtOAc) to afford the title compound as an off-white solid (0.053 g, 0.21 mmol, 29 %). ¹H NMR (DMSO-d₆ ): δ 3.93 (s, 3 H), 7.23 (m, 2 H), 7.98 (t, J=2.2 Hz, 1 H), 8.22 (d, J=5.0 Hz, 1 H), 8.28 (d, J=3.0 Hz, 1 H) 8.30 (d, J=1.2 Hz, 1 H), 12.65 (br s, 1 H).

### 5 -(2 -(5 -Methoxypyridin-3 -yl)-1 H-pyrrolo[2,3 -b]pyridin-4 -yl)-1 H-indazol-3 -amine SU1342

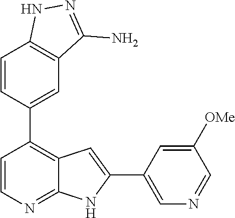 [SMILES: COc1cncc(-c2cc3c(-c4ccc5[nH]nc(N)c5c4)ccnc3[nH]2)c1]

To a suspension of 4 -chloro-2 -(5 -methoxypyridin-3 -yl)-1 H-pyrolo[2,3 -b]pyridine (0.049 g, 0.19 mmol), 5 -(4,4,5,5 -tetramethyl-1,3,2 -dioxaborolan-2 -yl)-1 H-indazol-3 -amine (0.062 g, 0.24 mmol) and [1,1 ′-bis(di-tert-butylphosphino)ferrocene]dichloropalladium(II) catalyst (0.015 g, 0.023 mmol) in EtOH (3.0 mL, degassed under nitrogen) was added K₃ PO₄ (1 M, 1.2 mL) and the reaction mixture was heated to 80° C. for 15 h. The reaction mixture was cooled to room temperature and diluted with EtOAc, washed with water, aqueous saturated NaHCO₃ and brine. The organics were concentrated under reduced pressure and the resulting residue and purified by column chromatography (100 % EtOAc—10 % MeOH/EtOAc—15 % MeOH/EtOAc) to afford the title compound as a light brown solid (0.008 g, 0.023 mmol, 12 %). ¹H NMR (DMSO-d₆ ): δ 3.93 (s, 3 H), 5.54 (br s, 2 H), 7,23 (d, J=5.0 Hz, 1 H), 7.38 (d, J=2.0 Hz, 1 H), 7.41 (d, J=6.8 Hz, 1 H), 7.74 (dd, J=1.5, 8.5 Hz, 1 H), 7.97 (t, J=2.2 Hz, 1 H), 8.25 (m, 2 H), 8.30 (d, J=5.0 Hz, 1 H), 8.84 (d, J=1.5 Hz, 1 H), 11.57 (br s, 1 H), 12.36 (br s, 1 H). HRMS: For C₂₀H₁₇0N₆ requires 357.1458 found 357.1453 . (M+H)⁺.

### Route to SU1343

### 4 -(5 -(4 -Chloro-1 H-pyrrolo[2,3 -b]pyridin-2 -yl)pyridin-2 -yl)morpholine

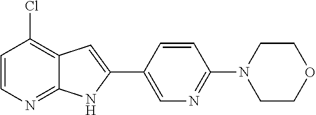 [SMILES: Clc1ccnc2[nH]c(-c3ccc(N4CCOCC4)nc3)cc12]

A suspension of 4 -chloro-2 -iodo-7 -azaindole (0.328 g, 1.18 mmol), 4 -(5 -(4,4,5,5 -tetramethyl-1,3,2 -dioxaborolan-2 -yl)pyridin-2 -yl)morpholine (0.40 g, 1.38 mmol), K₂ CO₃ (0.52 g, 3.76 mmol) and bis(triphenylphosphine) palladium(II) chloride (0.078 g, 0.11 mmol) in 1,4 -dioxane (3 mL) and water (2 mL) was degassed under nitrogen. The reaction mixture was heated to 100° C. for 17 h. The reaction mixture was cooled to room temperature and extracted into EtOAc, washed with water, aqueous saturated NaHCO₃ and brine. Organics were concentrated under reduced pressure and purified by column chromatography (100 % Hexane—1/1 Hexane/EtOAc 2/1 EtOAc/Hexane—4/1 EtOAc/Hexane—100 % EtOAc—10 % MeOH/EtOAc) to afford the title compound as an off-white solid (0.083 g). This material contained a slight trace of impurity and hence was used crude in the next reaction. ¹H NMR (DMSO-d₆ ): δ 3.54 (m, 4 H), 3.71 (m, 4 H), 6.87 (d, J=2.0 Hz, 1 H), 6.95 (d, J=9.2 Hz, 1 H), 7.17 (d, J=5.2 Hz, 1 H), 8.11 (d, J=5.2 Hz, 1 H), 8.14 (dd, J=2.8, 9.2 Hz, 1 H), 8.77 (d, J=2.4 Hz, 1 H), 12.38 (br s, 1 H).

### 5 -(2 -(6 -Morpholinopyridin-3 -yl)-1 H-pyrrolo[2,3 -b]pyridin-4 -yl)-1 H-indazol-3 -amine SU1343

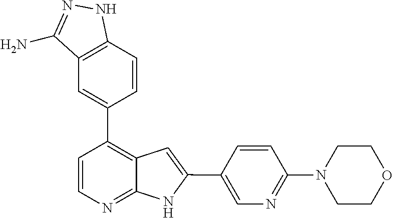 [SMILES: Nc1n[nH]c2ccc(-c3ccnc4[nH]c(-c5ccc(N6CCOCC6)nc5)cc34)cc12]

To a suspension of 4 -(5 -(4 -chloro-1 H-pyrrolo[2,3 -b]pyridin-2 -yl)pyridin-2 -yl)morpholine (0.077 g, 0.245 mmol), 5 -(4,4,5,5 -tetramethyl-1,3,2 -dioxaborolan-2 -yl)-1 H-indazol-3 -amine (0.099 g, 0.38 mmol) and [1,1 ′-bis(di-tert-butylphosphino)ferrocene]dichloropalladium(II) catalyst (0.025 g, 0.038 mmol) in EtOH (3.0 mL, degassed under nitrogen) was added K₃ PO₄ (1 M, 1.2 mL) and the reaction mixture was heated to 120° C. for 16 h. The reaction mixture was cooled to room temperature and diluted with EtOAc, washed with water, aqueous saturated NaHCO₃ and brine. The organics were concentrated under reduced pressure and the resulting residue and purified by column chromatography (100 % Hexane—1/1 Hexane/EtOAc—100 % EtOAc—10 % MeOH/EtOAc). The resulting solid was triturated with Et₂O to afford the title compound as a pale yellow solid (0.025 g, 0.06 mmol, 25 %). ¹H NMR (DMSO-d₆ ): δ 3.53 (t, J=4.8 Hz, 4 H), 3.72 (t, J=4.8 Hz, 4 H), 5.52 (br s, 2 H), 6.95 (d, J=9.2 Hz, 1 H), 7.07 (d, J=2.0 Hz, 1 H), 7.18 (d, J=4.8 Hz, 1 H), 7.39 (d, J=8.4 Hz, 1 H), 7.71 (dd, J=1.6, 8.8 Hz, 1 H), 8.14 (dd, J=2.4, 8.8 Hz, 1 H), 8.21 (d, J=5.2 Hz, 1 H), 8.22 (s, 1 H), 8.77 (d, J=2.0 Hz, 1 H), 11.53 (br s, 1 H), 12.10 (br s, 1 H). HRMS: For C₂₃H₂₂ON₇ requires 412.1876 found 412.1880 (M+H)⁺..

### Route to SU1343

### 4 -Chloro-2 -(6 -(4 -methylpiperazin-1 -yl)pyridin-3 -yl)-1 H-pyrrolo[2,3 -b]pyridine

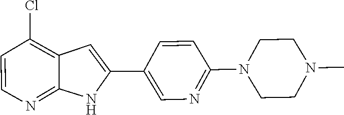 [SMILES: CN1CCN(c2ccc(-c3cc4c(Cl)ccnc4[nH]3)cn2)CC1]

A suspension of 4 -chloro-2 -iodo-7 -azaindole (0.328 g, 1.18 mmol), 1 -methyl-4 -(5 -(4,4,5,5 -tetramethyl-1,3,2 -dioxaborolan-2 -yl)pyridin-2 -yl)piperazine (0.43 g, 1.38 mmol), K₂ CO₃ (0.52 g, 3.76 mmol) and bis(triphenylphosphine) palladium(II) chloride (0.078 g, 0.11 mmol) in 1,4 -dioxane (3 mL) and water (2 mL) was degassed under nitrogen. The reaction mixture was heated to 100° C. for 17 h. The reaction mixture was cooled to room temperature and extracted into EtOAc, washed with water, aqueous saturated NaHCO₃ and brine. Organics were concentrated under reduced pressure and purified by column chromatography (100 % Hexane -1/1 Hexane/EtOAc 2/1 EtOAc/Hexane—4/1 EtOAc/Hexane—100 % EtOAc—10 % MeOH/EtOAc) to give 4 -chloro-2 -(6 -(4 -methylpiperazin-1 -yl)pyridin-3 -yl)-1 H-pyrrolo[2,3 -b]pyridine as an off-white solid (0.083 g). ¹H NMR (DMSO-d₆ ): δ 2.21 (S, 3 H), 2.39 (m, 4 H), 3.56 (m, 4 H), 6.83 (s, 1 H), 6.94 (d, J=9.2 Hz, 1 H), 7.15 (d, J=5.2 Hz, 1 H), 8.11 (m, 1 H), 8.73 (d, J=2.8 Hz, 1 H), 12.35 (br s, 1 H). LRMS found 238.20 (M+H)⁺calculated for C₁₇H₁₁ ClN₅ 327.13.

### 5 -(2 -(6 -(4 -Methylpiperazin-1 -yl)pyridin-3 -yl)-1 H-pyrrolo[2,3 -b]pyridin-4 -yl)-1 H-indazol-3 -amine SU1345

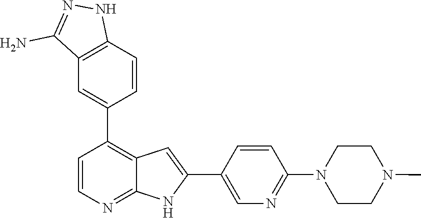 [SMILES: CN1CCN(c2ccc(-c3cc4c(-c5ccc6[nH]nc(N)c6c5)ccnc4[nH]3)cn2)CC1]

To a suspension of 4 -chloro-2 -(6 -(4 -methylpiperazin-1 -yl)pyridin-3 -yl)-1 H-pyrrolo[2,3 -b]pyridine (0.077 g, 0.245 mmol), 5 -(4,4,5,5 -tetramethyl-1,3,2 -dioxaborolan-2 -yl)-1 H-indazol-3 -amine (0.099 g, 0.38 mmol) and [1,1 ′-bis(di-tert-butylphosphino)ferrocene]dichloropalladium(II) catalyst (0.025 g, 0.038 mmol) in EtOH (3.0 mL, degassed under nitrogen) was added K₃ PO₄ (1 M, 1.2 mL) and the reaction mixture was heated to 120° C. for 16 h. The reaction mixture was cooled to room temperature and diluted with EtOAc, washed with water, aqueous saturated NaHCO₃ and brine. The organics were concentrated under reduced pressure and the resulting residue and purified by column chromatography (100 % Hexane—1/1 Hexane/EtOAc—100 % EtOAc—10 % MeOH/EtOAc). The resulting solid was triturated with Et₂O to afford the title compound as a pale yellow solid (0.018 g, 0.04 mmol, 13 %). ¹H NMR (DMSO-d₆ ): δ 2.85 (s, 3 H), 3.11 (m, 4 H), 3.51 (m, 2 H), 4.51 (m, 2 H) 7.07 (d, J=9.0 Hz, 1 H), 7.12 (s, 1 H), 7.46 (d, J=9.2 Hz, 1 H), 7.81 (d, J=5.2 Hz, 1 H), 8.12 (m, 1 H), 8.73 (d, J=4.8 Hz, 1 H), 8.28 (s, 1 H), 8.81 (d, J=2.8 Hz, 1 H), 12.28 (br s, 1 H), HRMS: For C₂₄H₂₄Nₐ requires 424.2124 found 425.2199 . (M+H)⁺.

### Route to SU1344

### 5 -(4 -Chloro-1 H-pyrrolo[2,3 -b]pyridin-2 -yl)-N-(2 -morpholinoethyl)pyridin-2 -amine

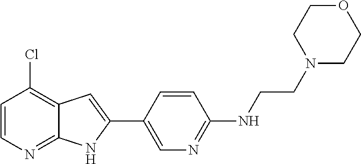 [SMILES: Clc1ccnc2[nH]c(-c3ccc(NCCN4CCOCC4)nc3)cc12]

A suspension of 4 -chloro-2 -iodo-7 -azaindole (0.337 g, 1.21 mmol), N-(2 -morpholinoethyl)-(5 -(4,4,5,5 -tetramethyl-1,3,2 -dioxaborolan-2 -yl)pyridin-2 -amine (0.456 g, 1.37 mmol), K₂ CO₃ (0.512 g, 3.70 mmol) and Bis(triphenylphosphine) palladium(II) chloride (0.074 g, 0.105 mmol) in 1,4 -dioxane (3 mL) and water (2 mL) was degassed under nitrogen. The reaction mixture was heated to 100° C. for 20 h. The reaction mixture was cooled to room temperature and extracted into EtOAc, washed with water, aqueous saturated NaHCO₃ and brine. Organics were concentrated under reduced pressure and the resulting solid triturated with hexane and Et₂O to afford the title compound. This material was used crude in the next reaction (0.512 g).

¹H NMR (DMSO-d₆ ): δ 2.42 (m, 4 H), 3.42 (q, J=6.3 Hz, 2 H), 3.59 (m, 4 H), 6.59 (d, J=8.8 Hz, 1 H), 6.75 (s, 1 H), 6.77 (t, J=5.4 Hz, 1 H), 7.14 (d, J=5.2 Hz, 1 H), 7.95 (dd, J=2.4, 8.8 Hz, 1 H), 8.08 (d, J=5.2 Hz, 1 H), 8.62 (d, J=2.0 Hz, 1 H), 12.27 (br s, 1 H).

### 5 -(2 -(6 -((2 -Morpholinoethyl)amino)pyridin-3 -yl)-1 H-pyrrolo[2,3 -b]pyridin-4 -yl)-1 H-indazol-3 -amine SU1344

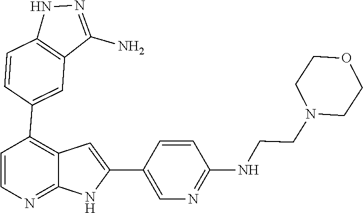 [SMILES: Nc1n[nH]c2ccc(-c3ccnc4[nH]c(-c5ccc(NCCN6CCOCC6)nc5)cc34)cc12]

To a suspension of 5 -(4 -chloro-1 H-pyrrolo[2,3 -b]pyridin-2 -yl)-N-(2 -morpholinoethyl)pyridin-2 -amine (0.106 g, 0.3 mmol), 5 -(4,4,5,5 -tetramethyl-1,3,2 -dioxaborolan-2 -yl)-1 H-indazol-3 -amine (0.103 g, 0.40 mmol) and [1,1 ′-bis(di-tert-butylphosphino)ferrocene]dichloropalladium(II) catalyst (0.025 g, 0.038 mmol) in EtOH (3.0 mL, degassed under nitrogen) was added K₃ PO₄ (1 M, 1.2 mL) and the reaction mixture was heated to 120° C. for 18 h. The reaction mixture was cooled to room temperature and diluted with EtOAc, washed with water, aqueous saturated NaHCO₃ and brine. The organics were then filtered through a pad of celite and concentrated under reduced pressure. The resulting residue was suspended in hexane EtOAc and dichloromethane and concentrated to 1/3 total volume to give a solid which was filtered under reduced pressure and dried to afford the title compound as an off white solid (0.014 g, 0.032 mmol, 11 %). ¹H NMR (DMSO-d₆ ): δ 2.43 (m, 4 H), 3.43 (q, J=6.4 Hz, 2 H), 3.60 (m, 4 H), 5.52 (br s, 2 H), 6.60 (d, J=8.8 Hz, 1 H), 6.68 (t, J=5.2 Hz, 1 H), 6.98 (d, J=2.0 Hz, 1 H), 7.17 (d, J=5.2 Hz, 1 H), 7.39 (d, J=8.4 Hz, 1 H), 7.72 (m, 1 H), 7.94 (dd, J=2.4, 8.8 Hz, 1 H), 8.19 (d, J=5.2 Hz, 1 H), 8.22 (s, 1 H), 8.63 (d, J=2.4 Hz, 1 H), 11.53 (br s, 1 H), 11.99 (br s, 1 H). HRMS: For C₂₅H₂₇ONs requires 455.2302 found 455.2299 . (M+H)⁺.

### 5 -(2 -(2 -Fluoropyridin-3 -yl)-1 H-pyrrolo[2,3 -b]pyridin-4 -yl)-1 H-indazol-3 -amine SU1356

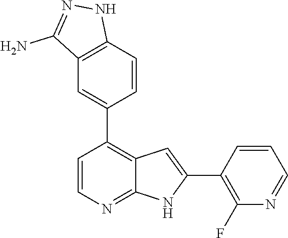 [SMILES: Nc1n[nH]c2ccc(-c3ccnc4[nH]c(-c5cccnc5F)cc34)cc12]

A 2-5 mL MW tube was charged with 4 -chloro-2 -(2 -fluoropyridin-3 -yl)-1 H-pyrrolo[2,3 -b]pyridine (196 mg, 0.8 mmol, 1 eq.), 5 -(4,4,5,5 -tetramethyl-1,3,2 -dioxaborolan-2 -yl)-1 H-indazol-3 -amine (269 mg, 1.04 mmol, 1.3 eq.), [1,1 ′-bis(di-tert-butylphosphino)ferrocene]dichloropalladium(II) catalyst (41.7 mg, 0.064 mmol, 8 mol %) in 1,4 -dioxane (4 mL). The resulting suspension was purged with nitrogen for 5 minutes before adding 2 M aq. K2 CO3 (1 mL). The reaction mixture was heated to 50° C. under a gentle flow of nitrogen for 10 minutes, then to 80° C. for 30 hours.

The stirred reaction mixture was then cooled to room temperature, slowly diluted with water (20 mL) and filtered. The filtered solid was then washed with water (10 mL×3 ) and hexane (10 mL×2 ) to give a crude (277 mg), which was purified by flash chromatography (SiO2, 75 g, 2-3 % MeOH in AcOEt) to afford the title compound as a yellow solid (138 mg, 50 %) ¹H NMR (400 MHz, DMSO-D₆ ) δ 5.55 (s, 2 H), 7.19-7.26 (m, 2 H), 7.42 (d, J=8.8 Hz, 1 H), 7.50-7.58 (m, 1 H), 7.69 (dd, J=8.7, 1.7 Hz, 1 H), 8.18-8.25 (m, 2 H), 8.35 (d, J=5.0 Hz, 1 H), 8.51-8.60 (m, 1 H), 11.59 (s, 1 H), 12.38 (s, 1 H).

### Route to SU1362

### 4 -(3 -(4 -Chloro-1 H-pyrrolo[2,3 -b]pyridin-2 -yl)pyridin-2 -yl)morpholine

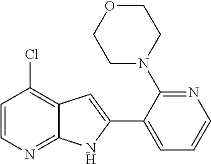 [SMILES: Clc1ccnc2[nH]c(-c3cccnc3N3CCOCC3)cc12]

A suspension of 4 -chloro-2 -iodo-7 -azaindole (0.276 g, 1.10 mmol), (2 -morpholinopyridin-3 -yl)boronic acid (0.227 g, 1.0 mmol), K₂ CO₃ (0.407 g, 2.94 mmol) and Bis(triphenylphosphine) palladium(II) chloride (0.069 g, 0.098 mmol) in 1,4 -dioxane (3 mL) and water (2 mL) was degassed under nitrogen. The reaction mixture was heated to 100° C. for 20 h. The reaction mixture was cooled to room temperature and extracted into EtOAc, washed with water, aqueous saturated NaHCO₃ and brine. Organics were concentrated under reduced pressure and the resulting solid purified by column chromatography (100 % Hexane—1/1 Hexane/EtOAc - 2/1 EtOAc/Hexane—100 % EtOAc) to afford the title compound as a colourless oil (0.127 g, 0.41 mmol, 31 %). ¹H NMR (DMSO-d₆ ): δ 3.04 (m, 4 H), 3.66 (m, 4 H), 6.95 (d, J=2.0 Hz, 1 H), 7.08 (m, 1 H), 7.21 (d, J=4.8 Hz, 1 H), 7.95 (dd, J=2.0, 7.6 Hz, 1 H), 8.18 (d, J=5.2 Hz, 1 H), 8.27 (dd, J=1.6, 4.8 Hz, 1 H), 12.30 (br s, 1 H).

### 5 -(2 -(2 -Morpholinopyridin-3 -yl)-1 H-pyrrolo[2,3 -b]pyridine-4 -yl)-1 H-indazol-3 -amine SU1362

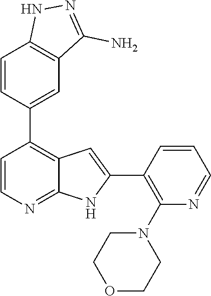 [SMILES: Nc1n[nH]c2ccc(-c3ccnc4[nH]c(-c5cccnc5N5CCOCC5)cc34)cc12]

To a suspension of 4 -(3 -(4 -chloro-1 H-pyrrolo[2,3 -b]pyridin-2 -yl)pyridin-2 -yl)morpholine (0.105 g, 0.33 mmol), 5 -(4,4,5,5 -tetramethyl-1,3,2 -dioxaborolan-2 -yl)-1 H-indazol-3 -amine (0.104 g, 0.40 mmol) and [1,1 ′-bis(di-tert-butylphosphino)ferrocene]dichloropalladium(II) catalyst (0.022 g, 0.033 mmol) in EtOH (3.0 mL, degassed under nitrogen) was added K₃ PO₄ (1 M, 1.2 mL) and the reaction mixture was heated to 120° C. for 20 h. The reaction mixture was cooled to room temperature, diluted with EtOAc, washed with water and brine. The organics were concentrated under reduced pressure and purified by column chromatography (100 % Hexane -100 % EtOAc—10 % MeOH/EtOAc) and trituration with Et₂O to afford the title compound as an off-white solid (0.035 g, 0.085 mmol, 26 %). ¹H NMR (DMSO-d₆ ): 5 3.06 (m, 4 H), 3.64 (m, 4 H), 5.51 (br s, 2 H), 7.08 (m, 1 H), 7.21 (t, J=4.0 Hz, 2 H), 7.40 (d, J=6.8 Hz, 1 H), 7.72 (dd, J=1.5, 8.5 Hz, 1 H), 8.00 (dd, J=2.0, 7.5 Hz, 1 H), 8.22 (s, 1 H), 8.25 (dd, J=1.5, 6.5 Hz, 1 H), 8.26 (d, J=5.0 Hz, 1 H), 11.55 (br s, 1 H), 11.98 (br s, 1 H). HRMS: For C₂₃H₂₂0N₇ requires 412.1880 found 412.1881 . (M+H)⁺.

(Hetero)Aromatic Moiety with Different Ether Substituents at Position 2 of the Azaindole Ring

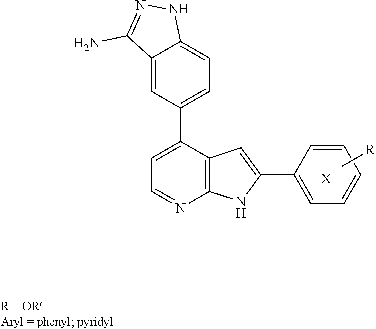

### Route to SU1367

### 4 -Chloro-2 -(3 -isobutoxyphenyl)-1 H-pyrrolo[2,3 -b]pyridine

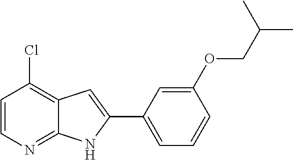 [SMILES: CC(C)COc1cccc(-c2cc3c(Cl)ccnc3[nH]2)c1]

A suspension of 4 -chloro-2 -iodo-7 -azaindole (0.291 g, 1.04 mmol), (3 -isobutylphenyl)boronic acid (0.259 g, 1.33 mmol), K₂ CO₃ (0.404 g, 2.92 mmol) and Bis(triphenylphosphine) palladium(II) chloride (0.064 g, 0.09 mmol) in 1,4 -dioxane (3 mL) and water (2 mL) was degassed under nitrogen. The reaction mixture was heated to 100° C. for 20 h. The reaction mixture was cooled to room temperature and extracted into EtOAc, washed with water, aqueous saturated NaHCO₃ and brine. Organics were filtered through a pad of celite and concentrated under reduced pressure. The resulting solid purified by recrystallization using dichloromethane and hexane to afford the title compound as an orange solid (0.14 g, 0.47 mmol, 45 %). ¹H NMR (DMSO-d₆ ): δ 1.02 (d, J=6.4 Hz, 6 H), 2.06 (m, 1 H), 3.85 (d, J=6.4 Hz, 2 H), 6.93 (m, 1 H), 7.04 (d, J=2.4 Hz, 1 H), 7.20 (d, J=5.2 Hz, 1 H), 7.37 (t, J=8.0 Hz, 1 H), 7.56 (s, 1 H), 7.58 (s, 1 H), 8.17 (d, J=5.2 Hz, 1 H), 12.48 (br s, 1 H).

### 5 -(2 -(3 -Isobutoxyphenyl)-1 H-pyrrolo[2,3 -b]pyridin-4 -yl)-1 H-indazol-3 -amine SU1367

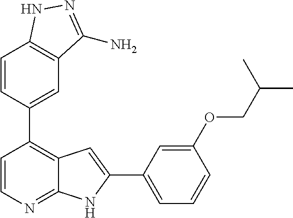 [SMILES: CC(C)COc1cccc(-c2cc3c(-c4ccc5[nH]nc(N)c5c4)ccnc3[nH]2)c1]

To a suspension of 4 -chloro-2 -(3 -isobutoxyphenyl)-1 H-pyrrolo[2,3 -b]pyridine (0.103 g, 0.34 mmol), 5 -(4,4,5,5 -tetramethyl-1,3,2 -dioxaborolan-2 -yl)-1 H-indazol-3 -amine (0.127 g, 0.49 mmol) and [1,1 ′-bis(di-tert-butylphosphino)ferrocene]dichloropalladium(II) catalyst (0.029 g, 0.05 mmol) in EtOH (3.0 mL, degassed under nitrogen) was added K₃ PO₄ (1 M, 1.2 mL) and the reaction mixture was heated to 120° C. for 21 h. The reaction mixture was cooled to room temperature, diluted with EtOAc, washed with water and brine. The organics were concentrated under reduced pressure, diluted into EtOAc, and filtered through a pad of celite. The solution was then concentrated under reduced pressure and purified by column chromatography (100 % Hexane—1/1 Hexane/EtOAc—2/1 EtOAc/Hexane—100 % EtOAc) to afford the title compound as an off-white solid (0.026 g, 0.06 mmol, 19 %). ¹H NMR (DMSO-d₆ ): δ 1.02 (d, J=6.8 Hz, 6 H), 2.06 (m, 1 H), 3.85 (d, J=6.4 Hz, 2 H), 5.54 (br s, 2 H), 6.91 (m, 1 H), 7.20 (m, 2 H), 7.39 (m, 2 H), 7.57 (m, 2 H), 7.72 (dd, J=1.6, 8.8 Hz, 1 H), 8.24 (s, 1 H), 8.26 (d, J=4.8 Hz, 1 H), 11.55 (br s, 1 H), 12.19 (br s, 1 H). HRMS: For C₂₄H₂₄0N₅ requires 398.1975 found 398.1970 . (M+H)⁺.

### Route to SU1335

### 2 -(3 -((Tetrahydro-2 H-pyran-4 -yl)methoxy)phenyl)-4 -chloro-1 H-pyrrolo[2,3 -b]pyridine

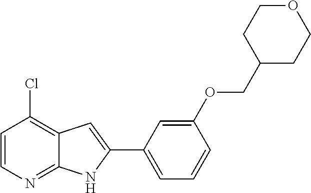 [SMILES: Clc1ccnc2[nH]c(-c3cccc(OCC4CCOCC4)c3)cc12]

4 -Chloro-2 -iodo-1 H-pyrrolo[2,3 -b]pyridine (0.24 g, 0.88 mmol), 3 -(tetrahydro-2 H-pyran-4 -yl)methoxyphenylboronic acid (0.250 g, 1.06 mmol), [1,1 ′-bis(diphenylphosphino)ferrocene]dichloropalladium (II) catalyst (0.036 g, 0.044 mmol) and a magnetic stirrer bar were added in a 10-20 mL microwave vial which was sealed with an aluminium crimp cap fitted with a disposable PTFE/silicon septum, and flushed with nitrogen. Then dioxane (10 mL) was added and the resulting mixture was purged with nitrogen for 10 min before adding 1.5 M K₂ CO₃ (aqueous solution, 1.6 mL). The mixture was purged again with nitrogen for 10 min and heated to 100° C. overnight. After cooling, the resulting mixture was filtered through celite and washed with EtOAc (20 mL). The filtrates were concentrated in vacuo to obtain the title compound as brown solid (0.241 g, 0.704 mmol, 80 %). ¹H NMR (400 MHz, DMSO-d₆ ) δ 1.31-1.42 (m, 2 H), 1.72 (d, J=11.6 Hz, 2 H), 2.00-2.08 (m, 1 H), 3.37 (dd, J=12.4, 1.6 Hz, 2 H), 3.89 (dd, J=11.2, 2.8 Hz, 2 H), 3.93 (d, J=6.8 Hz, 2 H), 6.94 (d, J=7.2 Hz, 1 H), 7.05 (d, J=2.0 Hz, 1 H), 7.20 (d, J=5.2 Hz, 1 H), 7.35-7.39 (m, 1 H), 7.56-7.58 (m, 2 H), 8.17 (d, J=5.2 Hz, 1 H), 12.51 (s, 1 H). LRMS found 183.13 (M+H)⁺calculated for C₁₂H₁₁N₂ 183.09

### 5 -(2 -(3 -((Tetrahydro-2 H-pyran-4 -yl)methoxy)phenyl)-1 H-pyrrolo[2,3 -b]pyridin-4 -yl)-1 H-indazol-3 -amine SU1335

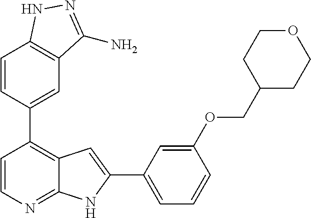 [SMILES: Nc1n[nH]c2ccc(-c3ccnc4[nH]c(-c5cccc(OCC6CCOCC6)c5)cc34)cc12]

2 -(3 -((tetrahydro-2 H-pyran-4 -yl)methoxy)phenyl)-4 -chloro-1 H-pyrrolo[2,3 -b]pyridine (0.24 g, 0.70 mmol), 5 -(4,4,5,5 -tetramethyl-1,3,2 -dioxaborolan-2 -yl)-1 H-indazol-3 -amine (0.22 g, 0.85 mmol), [1,1 ′-bis(di-tert-butylphosphino)ferrocene]dichloropalladium(II) catalyst (0.023 g, 0.04 mmol) and a magnetic stirrer bar were added in a 2-5 mL Biotage microwave vial which was sealed with an aluminium crimp cap fitted with a disposable PTFE/silicon septum, and flushed with nitrogen. Then EtOH (2.8 mL) was added, and the resulting mixture was purged with nitrogen for 10 min before adding 1 M K₃ PO₄ (aqueous solution, 1.4 mL). The mixture was purged again with nitrogen for 10 min and heated to 100° C. overnight. After cooling water was added to the reaction mixture and the solid that precipitated was filtered and dried in vacuo. The crude was purified by flash chromatography (SiO₂, EtOAc—EtOAc:MeOH 90:10 ) to obtain the title compound as white solid (0.050 g, 0.114 mmol, 16 %). ¹H NMR (400 MHz, DMSO-d₆ ) δ 1.31-1.42 (m, 2 H), 1.72 (d, J=11.2 Hz, 2 H), 1.95-2.12 (m, 1 H), 3.34-3.42 (m, 2 H), 3.90(dd, J=11.5, 3.4 Hz, 2 H), 3.94 (d, J=6.4 Hz, 2 H), 5.56 (s, 2 H), 6.92 (d, J=7.1 Hz, 1 H), 7.18 -7.21 (m, 2 H), 7.34-7.38 (m, 1 H), 7.40 (d, J=8.8 Hz, 1 H), 7.56-7.58 (m, 2 H), 7.72 (dd, J=8.8, 1.2 Hz, 1 H), 8.24 (s, 1 H), 8.26 (d, J=5.0 Hz, 1 H), 11.57 (s, 1 H), 12.22 (s, 1 H). HRMS (ESI) found 440.3 (MH)⁺calculated for C₂₆H₂₅N_{50 439.20.}

### Route to SU1336

### 4 -(2 -(3 -(4 -Chloro-1 H-pyrrolo[2,3 -b]pyridin-2 -yl)phenoxy)ethyl)morpholine

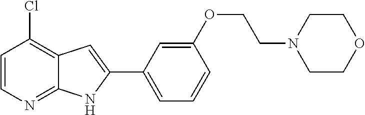 [SMILES: Clc1ccnc2[nH]c(-c3cccc(OCCN4CCOCC4)c3)cc12]

A suspension of 4 -chloro-2 -iodo-1 H-pyrrolo[2,3 -b]pyridine (0.25 g, 0.90 mmol), 3 -(2 -morpholinoethoxy)phenylboronic acid pinacol ester (0.359 g, 1.077 mmol), [1,1 ′-bis(diphenylphosphino)ferrocene]dichloropalladium II (0.037 g, 0.045 mmol) and a magnetic stirrer bar were added in a 10-20 mL microwave vial which was sealed with an aluminium crimp cap fitted with a disposable PTFE/silicon septum, and flushed with nitrogen. Then dioxane (10 mL) was added and the resulting mixture was purged with nitrogen for 10 min before adding 1.5 M K₂ CO₃ (aqueous solution, 1.7 mL). The mixture was purged again with nitrogen for 10 min and heated to 100° C. overnight. After cooling, the resulting mixture was filtered through celite and washed with EtOAc (30 mL). The filtrates were concentrated in vacuo and the residue was purified by flash chromatography (SiO₂, 1 % triethylamine in EtOAc—1 % triethylamine in EtOAc:MeOH 90:10 ) to obtain the title compound as beige solid (0.098 g, 0.275 mmol, 31 %). ¹H NMR (400 MHz, DMSO-d₆ ) 2.48-2.51 (m, 4 H), 2.73 (t, J=5.8 Hz, 2 H), 3.58-3.61 (m, 4 H), 4.19 (t, J=5.8 Hz, 2 H), 6.95 (dd, J=8.1, 2.0 Hz, 1 H), 7.06 (d, J=2.0 Hz, 1 H), 7.20 (d, J=5.2 Hz, 1 H), 7.35-7.39 (m, 1 H), 7.58 (d, J=8.0 Hz, 1 H), 7.60 (s, 1 H), 8.17 (d, J=5.2 Hz, 1 H), 12.49 (s, 1 H). LRMS found 358.1 (M+H)⁺ calculated for C₁₉H₂₁N₃O₂ CI 358.1

### 5 -(2 -(3 -(2 -Morpholinoethoxy)phenyl)-1 H-pyrrolo[2,3 -b]pyridin-4 -yl)-1 H-indazol-3 -amine SU1336

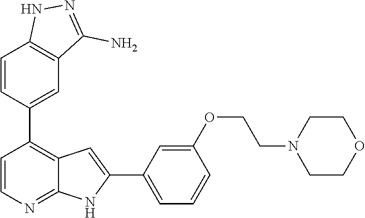 [SMILES: Nc1n[nH]c2ccc(-c3ccnc4[nH]c(-c5cccc(OCCN6CCOCC6)c5)cc34)cc12]

4 -(2 -(3 -(4 -Chloro-1 H-pyrrolo[2,3 -b]pyridin-2 -yl)phenoxy)ethyl)morpholine (0.08 g, 0.22 mmol), 5 -(4,4,5,5 -tetramethyl-1,3,2 -dioxaborolan-2 -yl)-1 H-indazol-3 -amine (0.070 g, 0.27 mmol), [1,1 ′-bis(di-tert-butylphosphino)ferrocene]dichloropalladium(II) catalyst (0.007 g, 0.011 mmol) and a magnetic stirrer bar were added in a 2-5 mL Biotage microwave vial which was sealed with an aluminium crimp cap fitted with a disposable PTFE/silicon septum, and flushed with nitrogen. Then EtOH (3 mL) was added, and the resulting mixture was purged with nitrogen for 10 min before adding 1 M K₃ PO₄ (aqueous solution, 0.5 mL). The mixture was purged again with nitrogen for 10 min and heated to 100° C. for 24 hours. After cooling water was added to the reaction mixture and the solid that precipitated was filtered and dried in vacuo. The crude was purified by flash chromatography (SiO₂, dichloromethane - dichlorormethane:MeOH 90:10 ) to obtain the title compound as white solid (0.074 g, 0.16 mmol, 73 %). ¹H NMR (400 MHz, DMSO-d₆ ) δ 2.48-2.51 (m, 4 H), 2.73 (t, J=5.7 Hz, 2 H), 3.56-3.63 (m, 4 H), 4.19 (t, J=5.7 Hz, 2 H), 5.56 (s, 2 H), 6.92 (d, J=8.4 Hz, 1 H), 7.20 (d, J=4.9 Hz, 1 H), 7.22 (d, J=2.1 Hz, 1 H), 7.34 -7.38 (m, 1 H), 7.40 (d, J=8.4 Hz, 1 H), 7.56-7.59 (m, 2 H), 7.73 (d, J=8.8 Hz, 1 H), 8.24 (s, 1 H), 8.26 (d, J=5.0 Hz, 1 H), 11.57 (s, 1 H), 12.20 (s, 1 H). HRMS (ESI) found 455.3 (MH)⁺ calculated for C₂₆H₂₆N₆O 454.21.

### 5 -(2 -(3 -(benzyloxy)phenyl)-1 H-pyrrolo[2,3 -b]pyridin-4 -yl)-1 H-indazol-3 -amine SU1261

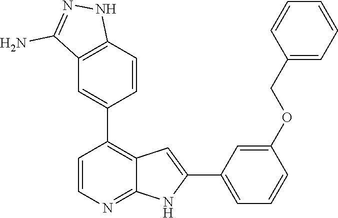 [SMILES: Nc1n[nH]c2ccc(-c3ccnc4[nH]c(-c5cccc(OCc6ccccc6)c5)cc34)cc12]

To a suspension of 2 -(3 -benzyloxy)phenyl)-4 -chloro-1 H-pyrrolo[2,3 -b]pyridine (0.084 g, 0.25 mmol), 5 -(4,4,5,5 -tetramethyl-1,3,2 -dioxaborolan-2 -yl)-1 H-indazol-3 -amine (0.097 g, 0.375 mmol) and [1,1 ′-bis(di-tert-butylphosphino)ferrocene]dichloropalladium(II) catalyst (0.008 g, 0.0125 mmol) in EtOH/water (1:1: 1.0 mL, degassed under nitrogen) was added K₃ PO₄ (1 M, 0.63 mL) and the reaction mixture was heated to 120° C. for 20 h. The reaction mixture was cooled to room temperature, diluted with EtOAc, washed with water and brine. The organics were concentrated under reduced pressure, diluted into EtOAc, and filtered through a pad of celite. The solution was then concentrated under reduced pressure and purified by column chromatography (100 % Hexane—1/1 Hexane/EtOAc—2/1 EtOAc/Hexane—100 % EtOAc) to afford the title compound as an off-white solid (0.095 g, 58 %). ¹H NMR (500 MHz, DMSO-d₆ ) δ 12.23 (s, 1 H), 11.59 (s, 1 H), 8.31-8.25 (m, 2 H), 7.75 (dd, J=8.6, 1.6 Hz, 1 H), 7.71 (t, J=2.0 Hz, 1 H), 7.61 (d, J=7.8 Hz, 1 H), 7.52 (d, J=7.5 Hz, 2 H), 7.41 (dt, J=14.4, 8.0 Hz, 4 H), 7.35 (t, J=7.4 Hz, 1 H), 7.28-7.18 (m, 2 H), 7.01 (dd, J=8.2, 2.4 Hz, 1 H), 5.58 (s, 2 H), 5.22 (s, 2 H). ¹³ C NMR (126 MHz, DMSO-d₆ ) δ 159.35, 150.94, 143.82, 141.71, 138.56, 137.53, 133.50, 130.46, 128.36, 127.20, 120.86, 118.9, 114.99, 112.13, 110.47, 97.79, 69.86 . LC-MS: exact mass calculated for C₂₇H₂₁N₅O: 431.17, found 432.3 (M+H)⁺..

### Route to SU1363

### 4 -Chloro-2 -(3 -((4 -methoxybenzyl)oxy)phenyl)-1 H-pyrrolo[2,3 -b]pyridine

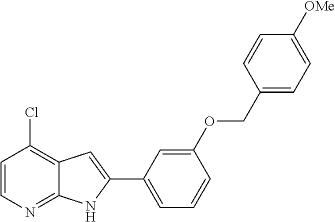 [SMILES: COc1ccc(COc2cccc(-c3cc4c(Cl)ccnc4[nH]3)c2)cc1]

A suspension of 4 -chloro-2 -iodo-7 -azaindole (0.358 g, 1.29 mmol), (3 -((4 -methoxybenzyl)oxy)phenyl)boronic acid (0.384 g, 1.49 mmol), K₂ CO₃ (0.537 g, 3.89 mmol) and Bis(triphenylphosphine) palladium(II) chloride (0.082 g, 0.12 mmol) in 1,4 -dioxane (3 mL) and water (2 mL) was degassed under nitrogen. The reaction mixture was heated to 100° C. for 20 h. The reaction mixture was cooled to room temperature and extracted into EtOAc, washed with water, aqueous saturated NaHCO₃ and brine. Organics were concentrated under reduced pressure and the resulting solid purified by column chromatography (100 % Hexane—3/1 Hexane/EtOAc—2/1 Hexane/EtOAc—1/1 Hexane/EtOAc—100 % EtOAc) and triturated with Et₂O to afford the title compound as an orange solid (0.237 g, 0.65 mmol, 51 %). ¹H NMR (DMSO-d₆ ): δ 3.75 (s, 3 H), 5.13 (s, 2 H), 6.98 (m, 3 H), 7.05 (s, 1 H), 7.21 (d, J=5.2 Hz, 1 H), 7.42 (m, 3 H), 7.58 (d, J=8.0 Hz, 1 H), 7.68 (t, J=2.0 Hz, 1 H), 8.17 (d, J=5.2 Hz, 1 H), 12.48 (br s, 1 H). MS (ESI) m/z (M+H)⁺.

### 5 -(2 -(3 -((4 -Methoxybenzyl)oxy)phenyl)-1 H-pyrrolo[2,3 -b]pyridin-4 -yl)-1 H-indazol-3 -amine SU1363

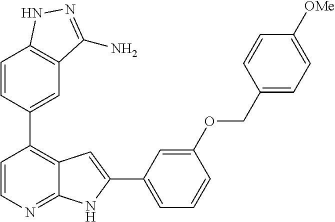 [SMILES: COc1ccc(COc2cccc(-c3cc4c(-c5ccc6[nH]nc(N)c6c5)ccnc4[nH]3)c2)cc1]

To a suspension of 4 -chloro-2 -(3 -((4 -methoxybenzyl)oxy)phenyl)-1 H-pyrrolo[2,3 -b]pyridine (0.10 g, 0.274 mmol), 5 -(4,4,5,5 -tetramethyl-1,3,2 -dioxaborolan-2 -yl)-1 H-indazol-3 -amine (0.111 g, 0.43 mmol) and [1,1 ′-bis(di-tert-butylphosphino)ferrocene]dichloropalladium(II) catalyst (0.018 g, 0.027 mmol) in EtOH (3.0 mL, degassed under nitrogen) was added K₃ PO₄ (1 M, 1.2 mL) and the reaction mixture was heated to 120° C. for 18 h. The reaction mixture was cooled to room temperature, diluted with EtOAc, washed with water and brine. The organics were concentrated under reduced pressure and purified by recrystallization using MeOH/DCM and hexane to afford the title compound as an off-white solid (0.081 g, 0.18 mmol, 64 %). ¹H NMR (DMSO-d₆ ): δ 3.75 (s, 3 H), 5.11 (s, 2 H), 5.54 (br s, 2 H), 6.98 (m, 3 H), 7.20 (m, 2 H), 7.39 (m, 4 H), 7.58 (d, J=8.0 Hz, 1 H), 7.66 (s, 1 H), 7.72 (dd, J=1.2, 8.4 Hz, 1 H), 8.24 (s, 1 H), 8.26 (d, J=5.2 Hz, 1 H), 11.55 (br s, 1 H), 12.19 (br s, 1 H). HRMS: For C₂₈H₂₄02N₅ requires 462.1925 found 462.1922 (M+H)⁺.

### Route to SU1364

### 4 -Chloro-2 -(3 -((3,5 -dimethoxybenzyl)oxy)phenyl)-1 H-pyrrolo[2,3 -b]pyridine

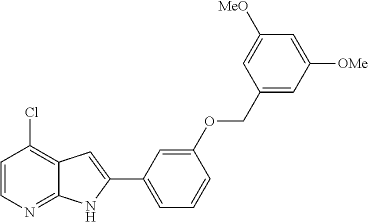 [SMILES: COc1cc(COc2cccc(-c3cc4c(Cl)ccnc4[nH]3)c2)cc(OC)c1]

A suspension of 4 -chloro-2 -iodo-7 -azaindole (0.309 g, 1.11 mmol), (3 -((3,5 -dimethoxybenzyl)oxy)phenyl)boronic acid (0.363 g, 1.26 mmol), K₂ CO₃ (0.455 g, 3.29 mmol) and Bis(triphenylphosphine) palladium(II) chloride (0.081 g, 0.11 mmol) in 1,4 -dioxane (3 mL) and water (2 mL) was degassed under nitrogen. The reaction mixture was heated to 100° C. for 20 h. The reaction mixture was cooled to room temperature and extracted into EtOAc, washed with water, aqueous saturated NaHCO₃ and brine. Organics were concentrated under reduced pressure and the resulting solid purified by column chromatography (100 % Hexane—3/1 Hexane/EtOAc—2/1 Hexane/EtOAc—1/1 Hexane/EtOAc—100 % EtOAc) and triturated with Et₂O to afford the title compound as an orange solid (0.215 g, 0.54 mmol, 49 %). ¹H NMR (DMSO-d₆ ): 5 3.75 (s, 6 H), 5.15 (s, 2 H), 6.46 (t, J=4.4 Hz, 1 H), 6.66 (d, J=2.4 Hz, 2 H), 7.01 (dd, J=2.0, 8.0 Hz, 1 H), 7.04 (d, J=2.0 Hz, 1 H), 7.21 (d, J=5.2 Hz, 1 H), 7.39 (t, J=8.0 Hz, 1 H), 7.59 (d, J=8.0 Hz, 1 H), 7.68 (t, J=3.6 Hz, 1 H), 8.18 (d, J=5.2 Hz, 1 H), 12.49 (br s, 1 H).

### 5 -(2 -(3 -((3,5 -dimethoxybenzyl)oxy)phenyl)-1 H-pyrrolo[2,3 -b]pyridin-4 -yl)-1 H-indazol-3 -amine SU 1364

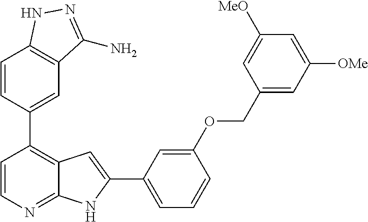 [SMILES: COc1cc(COc2cccc(-c3cc4c(-c5ccc6[nH]nc(N)c6c5)ccnc4[nH]3)c2)cc(OC)c1]

To a suspension of 4 -chloro-2 -(3 -((3,5 -dimethoxybenzyl)oxy)phenyl)-1 H-pyrrolo[2,3 -b]pyridine (0.104 g, 0.26 mmol), 5 -(4,4,5,5 -tetramethyl-1,3,2 -dioxaborolan-2 -yl)-1 H-indazol-3 -amine (0.118 g, 0.46 mmol) and [1,1 ′-bis(di-tert-butylphosphino)ferrocene]dichloropalladium(II) catalyst (0.018 g, 0.027 mmol) in EtOH (3.0 mL, degassed under nitrogen) was added K₃ PO₄ (1 M, 1.2 mL) and the reaction mixture was heated to 120° C. for 18 h. The reaction mixture was cooled to room temperature, diluted with EtOAc, washed with water and brine. The organics were concentrated under reduced pressure, diluted into EtOAc, and filtered through a pad of celite. The solution was then concentrated under reduced pressure and purified by recrystallization using MeOH/DCM and hexane to afford the title compound as a brown solid (0.084 g, 0.17 mmol, 65 %). ¹H NMR (DMSO-d₆ ): δ 3.74 (s, 6 H), 5.14 (s, 2 H), 5.53 (br s, 2 H), 6.45 (t, J=2.2 Hz, 1 H), 6.66 (d, J=2.4 Hz, 2 H), 6.98 (dd, J=2.0, 8.0 Hz, 1 H), 7.21 (t, J=3.6 Hz, 1 H), 7.38 (m, 2 H), 7.58 (d, J=8.0 Hz, 1 H), 7.67 (t, J=2.0 Hz, 1 H), 7.72 (dd, J=1.6, 0.8 Hz, 1 H), 8.23 (s, 1 H), 8.26 (d, J=5.2 Hz, 1 H), 11.55 (br s, 1 H), 12.20 (br s, 1 H). HRMS: For C₂₉H₂₆03N₅ requires 492.2030 found 492.2028.

### Route to SU1366

### 4 -Chloro-2 -(3 -((4 -fluorobenzyl)oxy)phenyl)-1 H-pyrrolo[2,3 -b]pyridine

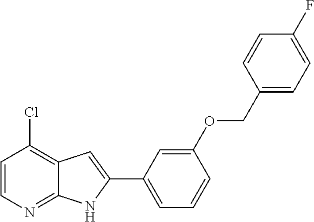 [SMILES: Fc1ccc(COc2cccc(-c3cc4c(Cl)ccnc4[nH]3)c2)cc1]

A suspension of 4 -chloro-2 -iodo-7 -azaindole (0.336 g, 1.21 mmol), (3 -((4 -fluorobenzyl)oxy)phenyl)boronic acid (0.341 g, 1.39 mmol), K₂ CO₃ (0.474 g, 3.43 mmol) and Bis(triphenylphosphine) palladium(II) chloride (0.084 g, 0.12 mmol) in 1,4 -dioxane (3 mL) and water (2 mL) was degassed under nitrogen. The reaction mixture was heated to 100° C. for 20 h. The reaction mixture was cooled to room temperature and extracted into EtOAc, washed with water, aqueous saturated NaHCO₃ and brine. Organics were filtered through a pad of celite and concentrated under reduced pressure. The resulting solid purified by recrystallization using dichloromethane and hexane to afford the title compound as an orange solid (0.28 g, 0.79 mmol, 66 %). ¹H NMR (DMSO-d₆ ): δ 5.20 (s, 2 H), 7.02 (dd, J=2.0, 8.0 Hz, 1 H), 7.05 (d, J=2.0 Hz, 1 H), 7.20 (d, J=5.2 Hz, 1 H), 7.34 (m, 2 H), 7.39 (t, J=8.0 Hz, 1 H), 7.56 (m, 2 H), 7.60 (d, J=7.6 Hz, 1 H), 7.70 (m, 1 H), 8.17 (d, J=5.2 Hz, 1 H), 12.51 (br s, 1 H).

### 5 -(2 -(3 -((4 -Fluorobenzyl)oxy)phenyl)-1 H-pyrrolo[2,3 -b]pyridin-4 -yl)-1 H-indazol-3 -amine SU1366

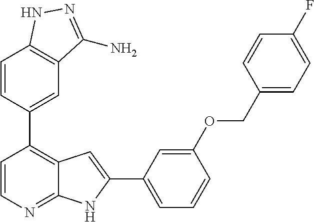 [SMILES: Nc1n[nH]c2ccc(-c3ccnc4[nH]c(-c5cccc(OCc6ccc(F)cc6)c5)cc34)cc12]

To a suspension of 4 -chloro-2 -(3 -((4 -fluorobenzyl)oxy)phenyl)-1 H-pyrrolo[2,3 -b]pyridine (0.103 g, 0.29 mmol), 5 -(4,4,5,5 -tetramethyl-1,3,2 -dioxaborolan-2 -yl)-1 H-indazol-3 -amine (0.116 g, 0.45 mmol) and [1,1 ′-bis(di-tert-butylphosphino)ferrocene]dichloropalladium(II) catalyst (0.022 g, 0.03 mmol) in EtOH (3.0 mL, degassed under nitrogen) was added K₃ PO₄ (1 M, 1.2 mL) and the reaction mixture was heated to 120° C. for 21 h. The reaction mixture was cooled to room temperature, diluted with EtOAc, washed with water and brine. The organics were concentrated under reduced pressure, diluted into EtOAc, and filtered through a pad of celite. The solution was then concentrated under reduced pressure and purified by column chromatography (100 % Hexane—1/1 Hexane/EtOAc—2/1 EtOAc/Hexane—100 % EtOAc) to afford the title compound as an off-white solid (0.014 g, 0.03 mmol, 11 %). ¹H NMR (DMSO-d₆ ): 8 5.19 (s, 2 H), 5.53 (br s, 2 H), 7.00 (dd, J=1.6, 7.6 Hz, 1 H), 7.24 (m, 4 H), 7.40 (m, 2 H), 7.56 (m, 3 H), 7.68 (m, 1 H), 7.72 (dd, J=1.2, 8.4 Hz, 1 H), 8.24 (s, 1 H), 8.27 (d, J=4.8 Hz, 1 H), 11.55 (br s, 1 H), 12.20 (br s, 1 H) HRMS: For C₂₇H₂₁0N₅ F requires 450.1725 found 450.1724

### Route to SU1369

### 4 -Chloro-2 -(3 -((2 -Fluorobenzyl)oxy)phenyl)-1 H-pyrrolo[2,3 -b]pyridine

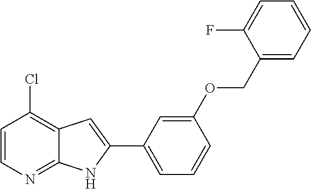 [SMILES: Fc1ccccc1COc1cccc(-c2cc3c(Cl)ccnc3[nH]2)c1]

A suspension of 4 -chloro-2 -iodo-7 -azaindole (0.312 g, 1.12 mmol), (3 -((2 -fluorobenzyl)oxy)phenyl)boronic acid (0.348 g, 1.42 mmol), K₂ CO₃ (0.464 g, 3.36 mmol) and Bis(triphenylphosphine) palladium(II) chloride (0.067 g, 0.10 mmol) in 1,4 -dioxane (3 mL) and water (2 mL) was degassed under nitrogen. The reaction mixture was heated to 100° C. for 20 h. The reaction mixture was cooled to room temperature and extracted into EtOAc, washed with water, aqueous saturated NaHCO₃ and brine. Organics were filtered through a pad of celite and concentrated under reduced pressure. The resulting solid was purified column chromatography (100 % Hexane—1/1 Hexane/EtOAc—2/1 EtOAc/Hexane—100 % EtOAc) to afford the title compound as an off-white solid (0.081 g, 0.23 mmol, 20 %). ¹H NMR (DMSO-d₆ ): δ 5.23 (s, 2 H), 7.04 (d, J=2.0, 8.0 Hz, 1 H), 7.07 (d, J=2.4 Hz, 1 H), 7.21 (d, J=5.2 Hz, 1 H), 7.28 )m, 2 H), 7.44 (M, 2 h), 7.63 (m, 2 H), 7.72 (t, J=2.0 Hz, 1 H), 8.18 (d, J=5.2 Hz, 1 H), 12.49 (br s, 1 H).

### 5 -(2 (3 -((2 -Fluorobenzyl)oxy)phenyl)-1 H-pyrrolo[2,3 -b]pyridin-4 -yl)-1 H-indazol-3 -amine SU1369

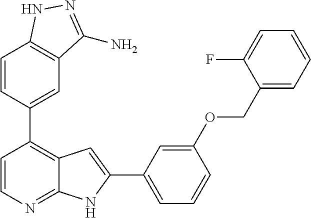 [SMILES: Nc1n[nH]c2ccc(-c3ccnc4[nH]c(-c5cccc(OCc6ccccc6F)c5)cc34)cc12]

To a suspension of 4 -chloro-2 -(3 -((2 -fluorobenzyl)oxy)phenyl)-1 H-pyrrolo[2,3 -b]pyridine (0.076 g, 0.22 mmol), 5 -(4,4,5,5 -tetramethyl-1,3,2 -dioxaborolan-2 -yl)-1 H-indazol-3 -amine (0.102 g, 0.39 mmol) and [1,1 ′-bis(di-tert-butylphosphino)ferrocene]dichloropalladium(li) catalyst (0.015 g, 0.02 mmol) in EtOH (3.0 mL, degassed under nitrogen) was added K₃ PO₄ (1 M, 1.2 mL) and the reaction mixture was heated to 120° C. for 20 h. The reaction mixture was cooled to room temperature, diluted with EtOAc, washed with water and brine. The organics were concentrated under reduced pressure, diluted into EtOAc, and filtered through a pad of celite. The solution was then concentrated under reduced pressure and purified by recrystallization from dichloromethane and hexane to afford the title compound as a brown solid (0.063 g, 0.14 mmol, 65 %). ¹H NMR (DMSO-d₆ ): δ 5.23 (s, 2 H), 5.53 (br s, 2 H), 7.01 (dd, J=2.0, 8.0 Hz, 1 H), 7.23 (m, 4 H), 7.43 (m, 3 H), 7.61 (d, J=6.8 Hz, 2 H), 7.72 (m, 2 H), 8.24 (s, 1 H), 8.26 (d, J=5.2 Hz, 1 H), 11.54 (br s, 1 H), 12.21 (br s, 1 H). HRMS: For C₂₇H₂₁0N₅ F requires 450.1725 found 450.1725.

### Route to SU1324

### 4 -Chloro-2 -(3 -(pyridin-2 -ylmethoxy)phenyl)-1 H-pyrrolo[2,3 -b]pyridine

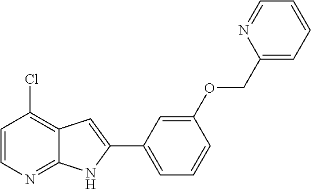 [SMILES: Clc1ccnc2[nH]c(-c3cccc(OCc4ccccn4)c3)cc12]

4 -Chloro-2 -iodo-1 H-pyrrolo[2,3 -b]pyridine (0.245 g, 0.88 mmol), 3 -(pyridin-2 -ylmethoxy)phenylboronic acid (0.24 g, 1.048 mmol), [1,1 ′-bis(diphenylphosphino)ferrocene]dichloropalladium II (0.036 g, 0.043 mmol) and a magnetic stirrer bar were added in a 10-20 mL microwave vial which was sealed with an aluminium crimp cap fitted with a disposable PTFE/silicon septum, and flushed with nitrogen. Then dioxane (10 mL) was added and the resulting mixture was purged with nitrogen for 10 min before adding 1.5 M K₂ CO₃ (aqueous solution, 1.6 mL). The mixture was purged again with nitrogen for 10 min and heated to 100° C. overnight. After cooling, the resulting mixture was filtered through celite and washed with EtOAc (20 mL). The filtrates were concentrated in vacuo to obtain the title compound as brown solid (0.270 g, 0.805 mmol, 92 %). ¹H NMR (400 MHz, DMSO-d₆ ) δ 5.30 (s, 2 H), 7.03-7.06 (m, 2 H), 7.21 (d, J=5.2 Hz, 1 H), 7.36 (d, J=7.2 Hz, 1 H), 7.37-7.45 (m, 1 H), 7.58 (d, J=7.8 Hz, 1 H), 7.61 (d, J=7.7 Hz, 1 H), 7.73 (s, 1 H), 7.84-7.88 (m, 1 H), 8.18 (d, J=5.2 Hz, 1 H), 8.60 (d, J=4.6 Hz, 1 H), 12.52 (s, 1 H). LRMS found 336.1 (M+H)⁺calculated for C₁₉H₁₅ ClN₃0 336.1

### 5 -(2 -(3 -(pyridin-2 -ylmethoxy)phenyl)-1 H-pyrrolo[2,3 -b]pyridin-4 -yl)-1 H-indazol-3 -amine SU1324

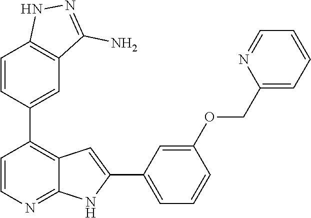 [SMILES: Nc1n[nH]c2ccc(-c3ccnc4[nH]c(-c5cccc(OCc6ccccn6)c5)cc34)cc12]

4 -Chloro-2 -(3 -(pyridin-2 -ylmethoxy)phenyl)-1 H-pyrrolo[2,3 -b]pyridine (0.268 g, 0.80 mmol), 5 -(4,4,5,5 -tetramethyl-1,3,2 -dioxaborolan-2 -yl)-1 H-indazol-3 -amine (0.290 g, 1.12 mmol), [1,1 ′-bis(di-tert-butylphosphino)ferrocene]dichloropalladium(II) catalyst (0.026 g, 0.04 mmol) and a magnetic stirrer bar were added in a 2-5 mL Biotage microwave vial which was sealed with an aluminium crimp cap fitted with a disposable PTFE/silicon septum, and flushed with nitrogen. Then EtOH (3.2 mL) was added, and the resulting mixture was purged with nitrogen for 10 min before adding 1 M K₃ PO₄ (aqueous solution, 1.6 mL). The mixture was purged again with nitrogen for 10 min and heated to 100° C. overnight. After cooling water was added to the reaction mixture and the solid that precipitated was filtered and dried in vacuo. The crude was purified by flash chromatography (SiO₂, EtOAc—EtOAc:MeOH 95:5 ) to obtain the title compound as beige solid (0.126 g, 0.291 mmol, 36 %). ¹H NMR (400 MHz, DMSO-d₆ ) δ 5.29 (s, 2 H), 5.57 (s, 2 H), 7.02 (d, J=8.0 Hz, 1 H), 7.20 (d, J=4.9 Hz, 1 H), 7.22 (d, J=1.9 Hz, 1 H), 7.34-7.42 (m, 3 H), 7.57-7.62 (m, 2 H), 7.75-7.69 (m, 2 H), 7.88-7.84 (m, 1 H), 8.24 (s, 1 H), 8.26 (d, J=5.0 Hz, 1 H), 8.60 (d, J=4.3 Hz, 1 H), 11.57 (s, 1 H), 12.24 (s, 1 H). HRMS found 433.1767 (M+H)⁺calculated for C₂₆H₂₁N₆O 433.1777

### Route to SU1384

### 3 -(4 -Chloro-1 H-pyrrolo[2,3 -b]pyridin-2 -yl)-5 -fluorophenol

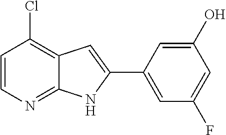 [SMILES: Oc1cc(F)cc(-c2cc3c(Cl)ccnc3[nH]2)c1]

A suspension of 4 -chloro-2 -iodo-7 -azaindole (0.305 g, 1.3 mmol), 3 -fluoro-5 -(4,4,5,5 -tetramethyl-1,3,2 -dioxaborolan-2 -yl)phenol (0.312 g, 1.3 mmol), ), K₂ CO₃ (0.53 g, 3.8 mmol) and Bis(triphenylphosphine) palladium(II) chloride (0.094 g, 0.134 mmol) in 1,4 -dioxane (3 mL) and water (2 mL) was degassed under nitrogen. The reaction mixture was heated to 100° C. for 18 h. The reaction mixture was cooled to room temperature and extracted into EtOAc, washed with water, aqueous saturated NaHCO₃ and brine. Organics were filtered through a pad of celite and concentrated under reduced pressure. The resulting solid was purified column chromatography (100 % Petrol - 2/1 Petrol/EtOAc—1/1 Petrol/EtOAc 100 % EtOAc) to afford the title compound as a yellow solid (0.0618 g, 0.24 mmol, 21.5 %). ¹H NMR (DMSO-d₆ ): δ 6.59 (dt, J=2.2, 10.4 Hz, 1 H), 6.98 (d, J=2.4 Hz, 1 H), 7.21 (m, 2 H), 7.30 (dt, J=1.8, 10.0 Hz, 1 H), 8.19 (d, J=4.8 Hz, 1 H), 10.13 (s, 1 H), 12.50 (br s, 1 H). LRMS: MS (+ve)=263.13.

### 3 -(4 -(3 -Amino-1 H-indazol-5 -yl)-1 H-pyrrolo[2,3 -b]pyridin-2 -yl)-5 -fluorophenol SU1384

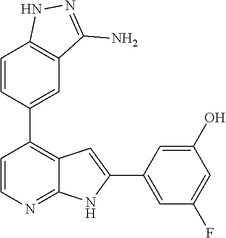 [SMILES: Nc1n[nH]c2ccc(-c3ccnc4[nH]c(-c5cc(O)cc(F)c5)cc34)cc12]

To a suspension of 3 -(4 -chloro-1 H-pyrrolo[2,3 -b]pyridin-2 -yl)-5 -fluorophenol (0.057 g, 0.22 mmol), ), 5 -(4,4,5,5 -tetramethyl-1,3,2 -dioxaborolan-2 -yl)-1 H-indazol-3 -amine (0.10 g, 0.39 mmol) and [1,1 ′-bis(di-tert-butylphosphino)ferrocene]dichloropalladium(II) catalyst (0.025 g, 0.038 mmol) in EtOH (3.0 mL, degassed under nitrogen) was added K₃ PO₄ (1 M, 1.2 mL) and the reaction mixture was heated to 120° C. for 22 h. The reaction mixture was cooled to room temperature, diluted with EtOAc, washed with water and brine. The organics were concentrated under reduced pressure, diluted into EtOAc, and filtered through a pad of celite. The solution was then concentrated under reduced pressure and the resulting residue was dissolved in DMF (2 mL) and filtered through cotton wool prior to purification by HPLC. Purification by HPLC (t_{R}=14 min) afforded the title compound as the TFA salt (0.0225 g, 0.048 mmol, 21.9 %) ¹H NMR (DMSO-d₆ ): δ 7.20 (d, J=2.0 Hz, 1 H), 7.24 (t, J=1.6 Hz, 1 H), 7.26 (d, J=4.8 Hz, 1 H), 7.29 (m, 1 H), 7.49 (d, J=8.4 Hz, 1 H), 7.85 (dd, J=1.6, 8.8 Hz, 1 H), 8.32 (d, J=4.8 Hz, 1 H), 8.35 (s, 1 H), 10.11 (s, 1 H), 12.35 (br s, 1 H). HRMS: For C₂₀H₁₅ON₅ F requires 360.1255 found 360.1252.

### 5 -(2 -(3 -Fluoro-5 -methoxyphenyl)-1 H-pyrrolo[2,3 -b]pyridin-4 -yl)-1 H-indazol-3 -amine SU1373

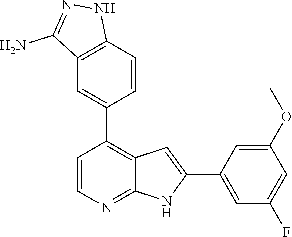 [SMILES: COc1cc(F)cc(-c2cc3c(-c4ccc5[nH]nc(N)c5c4)ccnc3[nH]2)c1]

A 2 -5 mL Biotage MW tube was charged with 4 -chloro-2 -(3 -fluoro-5 -methoxyphenyl)-1 H-pyrrolo[2,3 -b]pyridine (138 mg, 0.5 mmol, 1 eq.), 5 -(4,4,5,5 -tetramethyl-1,3,2 -dioxaborolan-2 -yl)-1 H-indazol-3 -amine (168 mg, 0.65 mmol, 1.3 eq.), [1,1 ′-bis(di-tert-butylphosphino)ferrocene]dichloropalladium(li) catalyst (16 mg, 0.025 mmol, 5 mol %) and 1,4 -dioxane (2.5 mL, oxygen free). The resulting suspension was purged with nitrogen for 5 minutes before adding 2 M aq. K₂ CO₃ (0.63 mL, oxygen free). The reaction mixture was heated to 50° C. under a gentle flow of nitrogen for 10 minutes, then to 110° C. for 24 hours. The stirred reaction mixture was then cooled to room temperature, slowly diluted with water (6 mL) and filtered. The filtered solid was then washed with water (3 mL×3 ) and hexane (3 mL×3 ).

Purification by flash chromatography gave the title compound as a yellow solid (84 mg, 45 %).

¹H NMR (400 MHz, DMSO-D₆ ) δ 3.86 (s, 3 H), 5.58 (s, 2 H), 6.77 -6.85 (m, 1 H), 7.22 (d, J=5.0 Hz, 1 H), 7.32 (s, 1 H), 7.40 (d, J=8.7 Hz, 1 H), 7.43 -7.50 (m, 2 H), 7.73 (d, J=8.7 Hz, 1 H), 8.24 (s, 1 H), 8.29 (d, J=4.9 Hz, 1 H), 11.59 (s, 1 H), 12.31 (s, 1 H). LRMS: Calculated for C₂₁H₁₆FN₅O 373.1339 ; Found:374.3 (M+H)⁺.

### Route to SU1385

### 4 -Chloro-2 -(3 -fluoro-5 -(2 -methoxyethoxy)pheny)-1 H-pyrrolo[2,3 -b]pyridine

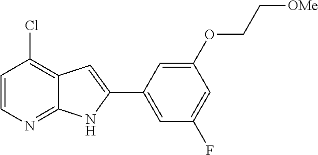 [SMILES: COCCOc1cc(F)cc(-c2cc3c(Cl)ccnc3[nH]2)c1]

A suspension of 2 -(3 -fluoro-5 -(2 -methoxyethoxy)phenyl)-4,4,5,5 -tetramethyl-1,3,2 -dioxaborolane (0.37 g, 1.26 mmol), 4 -chloro-2 -iodo-7 -azaindole (0.301 g, 10.8 mmol), K2 CO₃ (0.513 g, 3.71 mmol) and Bis(triphenylphosphine) palladium(II) chloride (0.096 g, 0.14 mmol) in 1,4 -dioxane (3 mL) and water (2 mL) was degassed under nitrogen. The reaction mixture was heated to 100° C. for 18 h. The reaction mixture was cooled to room temperature and extracted into EtOAc, washed with water, aqueous saturated NaHCO₃ and brine. Organics were filtered through a pad of celite and concentrated under reduced pressure. The resulting solid was purified column chromatography (100 % Petrol - 2/1 Petrol/EtOAc—100 % EtOAc) to afford the title compound as an off-white solid which was used in the next step without further purification (0.745 g). ¹H NMR (DMSO-d₆ ): 3.33 (s, 1 H), 3.67 (m, 2 H), 4.23 (m, 2 H), 6.85 (dt, J=2.2, 10.8 Hz, 1 H), 7.18 (d, J=2.4 Hz, 1 H), 7.22 (d, J=5.2 Hz, 1 H), 7.34 (m, 1 H), 7.49 (m, 2 H), 8.20 (d, J=5.2 Hz, 1 H), 12.53 (br s, 1 H). LRMS: MS (+ve)=321.13.

### 5 -(2 -(3 -Fluoro-5 -(2 -methoxyethoxy)phenyl)-1 H-pyrrolo[2,3 -b]pyridin-4 -yl)-1 H-indazol-3 -amine SU1385

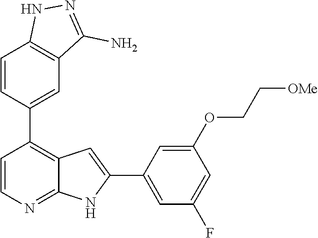 [SMILES: COCCOc1cc(F)cc(-c2cc3c(-c4ccc5[nH]nc(N)c5c4)ccnc3[nH]2)c1]

To a suspension of 4 -chloro-2 -(3 -fluoro-5 -(2 -methoxyethoxy)pheny)-1 H-pyrrolo[2,3 -b]pyridine (0.066 g, 0.21 mmol), 5 -(4,4,5,5 -tetramethyl-1,3,2 -dioxaborolan-2 -yl)-1 H-indazol-3 -amine (0.096 g, 0.37 mmol) and [1,1 ′-bis(di-tert-butylphosphino)ferrocene]dichloropalladium(II) catalyst (0.022 g, 0.034 mmol) in EtOH (3.0 mL, degassed under nitrogen) was added K₃ PO₄ (1 M, 1.2 mL) and the reaction mixture was heated to 120° C. for 20 h. The reaction mixture was cooled to room temperature, diluted with EtOAc, washed with water and brine. The organics were concentrated under reduced pressure, diluted into EtOAc, and filtered through a pad of celite. The solution was then concentrated under reduced pressure and the resulting residue was dissolved in DMF (2 mL) and filtered through cotton wool prior to purification by HPLC. Purification by HPLC (t_{R}=17 min) afforded the title compound as the double TFA salt (0.0325 g, 0.05 mmol, 24.4 %). ¹H NMR (DMSO-d₆ ): δ 3.30 (s, 3 H), 3.67 (m, 2 H), 4.21 (m, 2 H), 6.84 (dt, J=2.2, 11.2 Hz, 1 H), 7.26 (d, J=5.2 Hz, 1 H), 7.34 (d, J=2.0 Hz, 1 H), 7.48 (m, 3 H), 7.86 (dd, J=1.2, 8.8 Hz, 1 H), 8.32 (d, J=4.8 Hz, 1 H), 8.34 (s, 1 H), 12.36 (br s, 1 H). HRMS: For C₂₃H₂₁O₂N₅ F requires 418.1674 found 418.1671 . (M+H)⁺.

### Route to SU1389

### 4 -Chloro-2 -(3 -fluoro-5 -((tetrahydro-2 H-pyran-4 -yl)methoxy)phenyl)-1 H-pyrrolo[2,3 -b]pyridine

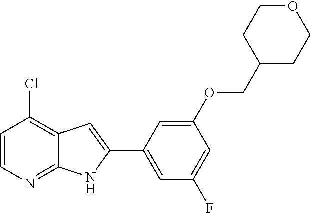 [SMILES: Fc1cc(OCC2CCOCC2)cc(-c2cc3c(Cl)ccnc3[nH]2)c1]

A suspension of 2 -(3 -fluoro-5 -((tetrahydro-2 H-pyran-4 -yl)methoxy)phenyl)-4,4,5,5 -tetramethyl-1,3,2 -dioxaborolane (0.425 g, 1.26 mmol), 4 -chloro-2 -iodo-7 -azaindole (0.331 g, 1.19 mmol), ), K₂ CO₃ (0.51 g, 3.7 mmol) and Bis(triphenylphosphine) palladium(II) chloride (0.098 g, 0.14 mmol) in 1,4 -dioxane (3 mL) and water (2 mL) was degassed under nitrogen. The reaction mixture was heated to 100° C. for 20 h. The reaction mixture was cooled to room temperature and extracted into EtOAc, washed with water, aqueous saturated NaHCO₃ and brine. Organics were filtered through a pad of celite and concentrated under reduced pressure. The resulting solid was purified column chromatography (100 % Petrol - 2/1 Petrol/EtOAc—1/1 Petrol/EtOAc - 100 % EtOAc) to afford the title compound as an orange solid (0.101 g, 0.28 mmol, 18 %). ¹H NMR (DMSO-d₆ ): δ 1.35 (m, 2 H), 1.70 (m, 2 H), 2.04 (m, 1 H), 3.38 (m, 2 H), 3.89 (m, 2 H), 3.97 (d, J=5.2 Hz, 2 H), 6.82 (dt, J=1.8, 8.4 Hz, 1 H), 7.16 (d, J=2.0 Hz, 1 H), 7.22 (d, J=3.2 Hz, 1 H), 7.48 (m, 2 H), 8.20 (d, J=4.4 Hz, 1 H), 12.53 (br s, 1 H).

### 5 -(2 -(3 -Fluoro-5 -((tetrahydro-2 H-pyran-4 -yl)methoxy)phenyl)-1 H-pyrrolo[2,3 -b]pyridine-4 -yl)-1 H-indazol-3 -amine SU1389

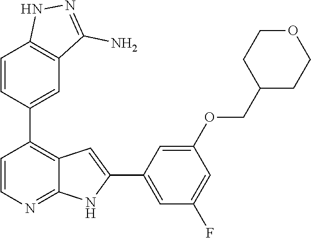 [SMILES: Nc1n[nH]c2ccc(-c3ccnc4[nH]c(-c5cc(F)cc(OCC6CCOCC6)c5)cc34)cc12]

To a suspension of 4 -chloro-2 -(3 -fluoro-5 -((tetrahydro-2 H-pyran-4 -yl)methoxy)phenyl)-1 H-pyrrolo[2,3 -b]pyridine (0.093 g, 0.26 mmol), 5 -(4,4,5,5 -tetramethyl-1,3,2 -dioxaborolan-2 -yl)-1 H-indazol-3 -amine (0.10 g, 0.36 mmol) and [1,1 ′-bis(di-tert-butylphosphino)ferrocene]dichloropalladium(II) catalyst (0.026 g, 0.040 mmol) in EtOH (3.0 mL, degassed under nitrogen) was added K₃ PO₄ (1 M, 1.2 mL) and the reaction mixture was heated to 120° C. for 18 h. The reaction mixture was cooled to room temperature, diluted with EtOAc, washed with water and brine. The organics were concentrated under reduced pressure, diluted into EtOAc, and filtered through a pad of celite. The solution was then concentrated under reduced pressure and the resulting residue was purified using column chromatography (100 % Petrol - 1/1 Petrol/EtOAc—1/2 Petrol/EtOAc—100 % EtOAc—5 % MeOH/EtOAc—10 % MeOH/EtOAc). The fractions containing the product were concentrated under reduced pressure and the resulting residue was suspended in MeOH to which petrol was added. The solution was then concentrated to 1/3 total volume and the resulting solid was filtered and dried to afford the title compound as a pale solid (0.009 g, 0.020 mmol, 8 %). This was confirmed by NMR to be a 1:1 mixture of desired product and corresponding phenol. Analysis of desired product only: ¹H NMR (DMSO-d₆ ): δ 0.85 (m, 2 H), 1.35 (m, 2 H), 1.71 (m, 1 H), 2.04 (m, 1 H), 3.36 (m, 1 H), 3.89 (m, 1 H), 3.96 (d, J=6.8 Hz, 1 H), 6.60 (m, 1 H), 7.24 (m, 2 H), 7.30 (d, J=2.0 Hz, 1 H), 7.41 (m, 2 H), 7.72 (m, 1 H), 8.24 (d, J=3.2 Hz, 1 H), 8.28 (t, J=4.6 Hz, 1 H), 11.55 (br s, 1 H).HRMS: For C₂₆H₂₅02N₅ F requires 458.1987 found 458.1986 . (M+H)⁺.

### 5 -(2 -(3 -(Benzyloxy)-5 -fluorophenyl)-1 H-pyrrolo[2,3 -b]pyridin-4 -yl)-1 H-indazol-3 -amine SU1371

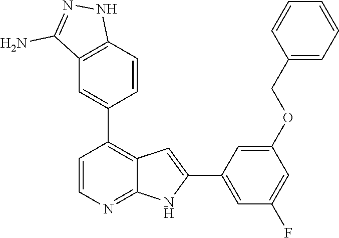 [SMILES: Nc1n[nH]c2ccc(-c3ccnc4[nH]c(-c5cc(F)cc(OCc6ccccc6)c5)cc34)cc12]

A 2 -5 mL Biotage MW tube was charged with 2 -(3 -(benzyloxy)-5 -fluorophenyl)-4 -chloro-1 H-pyrrolo[2,3 -b]pyridine (106 mg, 0.3 mmol, 1 eq.), 5 -(4,4,5,5 -tetramethyl-1,3,2 -dioxaborolan-2 -yl)-1 H-indazol-3 -amine (101 mg, 0.101 mmol, 1.3 eq.), [1,1 ′-bis(di-tert-butylphosphino)ferrocene]dichloropalladium(II) catalyst (9.8 mg, 0.015 mmol, 5 mol %) and ethanol (1.8 mL, oxygen free). The resulting suspension was purged with nitrogen for 5 minutes before adding 1 M aq. K₃ PO₄ (1.8 mL). The reaction mixture was heated to 40° C. under a gentle flow of nitrogen for 10 minutes, then to 110° C. for 24 hours. The stirred reaction mixture was then cooled to room temperature, slowly diluted with water (7 mL) and filtered. The filtered solid was then washed with water (3 mL×3 ) and hexane (3 mL×3 ). The crude was recrystallized from AcOEt to give the title compound as a beige solid (71 mg). ¹H NMR (400 MHz, DMSO-D₆ ) δ 5.22 (s, 2 H), 5.56 (s, 2 H), 6.90 (d, J=10.7 Hz, 1 H), 7.22 (d, J=4.9 Hz, 1 H), 7.31 -7.61 (m, 9 H), 7.73 (d, J=8.4 Hz, 1 H), 8.23 (s, 1 H), 8.29 (d, J=4.9 Hz, 1 H), 11.58 (s, 1 H), 12.27 (s, 1 H). HRMS: Calculated for C₂₇H₂₀FN₅0 449.1652 ; Found:450.3 (M+H)⁺.

### 5 -(2 -(3 -(Benzyloxy)-5 -(trifluoromethoxy)phenyl)-1 H-pyrrolo[2,3 -b]pyridin-4 -yl)-1 H-indazol-3 -amine SU1355

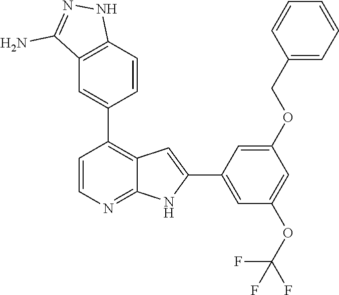 [SMILES: Nc1n[nH]c2ccc(-c3ccnc4[nH]c(-c5cc(OCc6ccccc6)cc(OC(F)(F)F)c5)cc34)cc12]

A 2 -5 mL Biotage MW tube was charged with 2 -(3 -(benzyloxy)-5 -(trifluoromethoxy)phenyl)-4 -chloro-1 H-pyrrolo[2,3 -b]pyridine (100 mg, 0.3 mmol), 5 -(4,4,5,5 -tetramethyl-1,3,2 -dioxaborolan-2 -yl)-1 H-indazol-3 -amine (101 mg, 0.4 mmol, 1.3 eq.), [1,1 ′-bis(di-tert-butylphosphino)ferrocene]dichloropalladium(II) catalyst (9.8 mg, 0.015 mmol, 5 mol %) and dioxane (1.8 mL). The resulting suspension was purged with nitrogen for 5 minutes before adding 1 M aq. K₃ PO₄ (1.8 mL). The reaction mixture was heated to 110° C. for 16 hrs. The reaction mixture was then allowed to cool to room temperature, before diluted with water (10 mL) and extracted with EtOAc (3×20 mL). The combined organic fractions were then dried over anhydrous magnesium sulfate, filtered and the solvent removed under reduced pressure. The crude product was then purified by flash chromatography (100 % EtOAc to 10 % MeOH in EtOAc) to give the desired product as a pale yellow solid (21 mg). ¹H NMR (500 MHz, DMSO-D₆ ) δ 5.24 (s, 2 H), 5.53 (s, 2 H), 6.99 (s, 1 H), 7.22 (d, J=4.9 Hz, 1 H), 7.31 -7.41 (m, 5 H), 7.51 (d, J=8.4 Hz, 1 H), 7.61 (s, 1 H), 7.73 (m, 2 H), 8.22 (s, 1 H), 8.29 (d, J=4.9 Hz, 1 H), 11.55 (s, 1 H), 12.32 (s, 1 H). HRMS: Calculated for C₂₈H₂₀ F3N₅O₂ 515.1569 ; Found:516.1642 (M+H)⁺.

### Methyl 3 -(4 -(3 -amino-1 H-indazol-5 -yl)-1 H-pyrrolo[2,3 -b]pyridin-2 -yl)-5 -(benzyloxy)benzoate SU1360

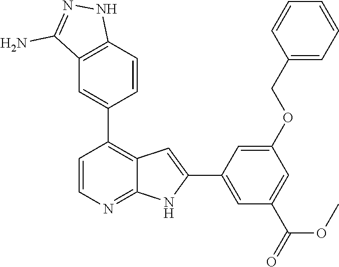 [SMILES: COC(=O)c1cc(OCc2ccccc2)cc(-c2cc3c(-c4ccc5[nH]nc(N)c5c4)ccnc3[nH]2)c1]

A 2 -5 mL Biotage MW tube was charged with methyl 3 -(benzyloxy)-5 -(4 -chloro-1 H-pyrrolo[2,3 -b]pyridin-2 -yl)benzoate (100 mg, 0.3 mmol), 5 -(4,4,5,5 -tetramethyl-1,3,2 -dioxaborolan-2 -yl)-1 H-indazol-3 -amine (103 mg, 0.4 mmol, 1.3 eq.), [1 ,1 ′-bis(di-tert-butylphosphino)ferrocene]dichloropalladium(II) catalyst (9.8 mg, 0.015 mmol, 5 mol %) and dioxane (1.8 mL). The resulting suspension was purged with nitrogen for 5 minutes before adding 1 M aq. K₃ PO₄ (1.8 mL). The reaction mixture was heated to 110 00 for 16 hrs. The reaction mixture was then allowed to cool to room temperature, diluted with water (10 mL) and extracted with EtOAc (3×20 mL). The combined organic fractions were then dried over anhydrous magnesium sulfate, filtered and the solvent removed under reduced pressure. The crude product was then purified by HPLC to give the desired product as a pale yellow solid (21 mg). ¹H NMR (500 MHz, DMSO-D₆ ) δ 3.89 (s, 2 H), 5.28 (s, 2 H), 5.42 (s, 2 H), 7.15 (s, 1 H), 7.21 (d, J=4.9 Hz, 1 H), 7.35 -7.45 (m, 6 H), 7.51 (d, J=8.4 Hz, 1 H), 7.61 (s, 1 H), 7.73 (m, 2 H), 8.22 (s, 1 H), 8.29 (d, J=4.9 Hz, 1 H), 11.55 (s, 1 H), 12.32 (s, 1 H). LRMS: Calculated for C₂₉H₂₃N₅O₃ 489.18 ; Found:490.20 (M+H)⁺.

### 5 -(2 -(3 -(Benzyloxy)-5 -((2 -methoxyethoxy)methyl)phenyl)-1 H-pyrrolo[2,3 -b]pyridin-4 -yl)-1 H-indazol-3 -amine SU1358

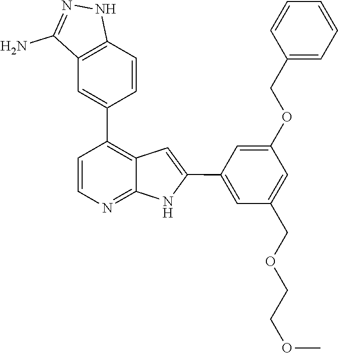 [SMILES: COCCOCc1cc(OCc2ccccc2)cc(-c2cc3c(-c4ccc5[nH]nc(N)c5c4)ccnc3[nH]2)c1]

A 0.5 -2 mL Biotage MW tube was charged with 2 -(3 -(benzyloxy)-5 -((2 -methoxyethoxy)methyl)phenyl)-4 -chloro-1 H-pyrrolo[2,3 -b]pyridine (33 mg, 0.078 mmol, 1 eq.), 5 -(4,4,5,5 -tetramethyl-1,3,2 -dioxaborolan-2 -yl)-1 H-indazol-3 -amine (26 mg, 0.101 mmol, 1.3 eq.), [1,1 ′-bis(di-tert-butylphosphino)ferrocene]dichloropalladium(II) catalyst (2.5 mg, 0.0039 mmol, 5 mol %) and 1,4 -dioxane (624 μL, oxygen free). The resulting suspension was purged with nitrogen for 5 minutes before adding 1 M aq. K₃ PO₄ (624 μL, oxygen free). The reaction mixture was heated to 50° C. under a gentle flow of nitrogen for 10 minutes, then to 110° C. for 24 hours. The stirred reaction mixture was then cooled to room temperature, slowly diluted with water (6 mL) and filtered. The filtered solid was then washed with water (3 mL×3 ) and hexane (3 mL×3 ) to give the title compound as an off-white solid (31 mg, 76 %). ¹H NMR (400 MHz, DMSO-D₆ ) δ 3.25 (s, 3 H), 3.46 -3.53 (m, 2 H), 3.55 -3.60 (m, 2 H), 4.52 (s, 2 H), 5.20 (s, 2 H), 5.57 (s, 2 H), 6.97 (s, 1 H), 7.17 -7.22 (m, 2 H), 7.32 -7.38 (m, 1 H), 7.38 -7.46 (m, 3 H), 7.51 (d, J=7.5 Hz, 2 H), 7.55 (s, 1 H), 7.61 (s, 1 H), 7.72 (d, J=8.8 Hz, 1 H), 8.23 (s, 1 H), 8.26 (d, J=4.9 Hz, 1 H), 11.57 (s, 1 H), 12.23 (s, 1 H).

### 5 -(2 -(3 -(Benzyloxy)-5 -((2 -methoxyethyl)amino)phenyl)-1 H-pyrrolo[2,3 -b]pyridin-4 -yl)-1 H-indazol-3 -amine SU1365

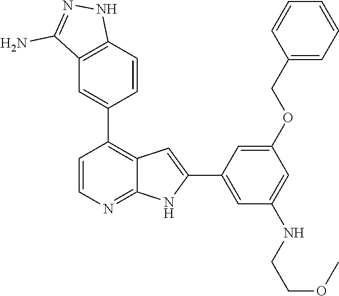 [SMILES: COCCNc1cc(OCc2ccccc2)cc(-c2cc3c(-c4ccc5[nH]nc(N)c5c4)ccnc3[nH]2)c1]

A 2 -5 mL Biotage MW tube was charged with methyl 3 -(benzyloxy)-5 -(4 -chloro-1 H-pyrrolo[2,3 -b]pyridin-2 -yl)-N-(2 -methoxyethyl)aniline (100 mg, 0.3 mmol), 5 -(4,4,5,5 -tetramethyl-1,3,2 -dioxaborolan-2 -yl)-1 H-indazol-3 -amine (103 mg, 0.4 mmol, 1.3 eq.), [1,1 ′-bis(di-tert-butylphosphino)ferrocene]dichloropalladium(II) catalyst (9.8 mg, 0.015 mmol, 5 mol %) and dioxane (1.8 mL). The resulting suspension was purged with nitrogen for 5 minutes before adding 1 M aq. K₃ PO₄ (1.8 mL). The reaction mixture was heated to 110° C. for 16 hrs. The reaction mixture was then allowed to cool to room temperature, diluted with water (10 mL) and extracted with EtOAc (3×20 mL). The combined organic fractions were then dried over anhydrous magnesium sulfate, filtered and the solvent removed under reduced pressure. The crude product was then purified by HPLC to give the desired product as a pale yellow solid (21 mg). ¹H NMR (500 MHz, DMSO-D₆ ) δ 3.27 (m, 7 H), 3.50 (m, 2 H), 5.11 (s, 2 H), 6.83 (s, 1 H), 6.90 (s, 1 H), 7.07 (d, J=1.7 Hz, 1 H), 7.21 (d, J=4.9 Hz, 1 H), 7.32 -7.49 (m, 6 H), 7.82 (d, J=8.4 Hz, 1 H), 8.27 (d, J=4.9 Hz, 1 H), 8.31 (s, 1 H), 12.23 (s, 1 H). LRMS: Calculated for C₂₀H₂₈N₆O₂ 504.23 ; Found:505.30 (M+H)⁺.

### 5 -(2 -(3 -(benzyloxy)-5 -((2 -morpholinoethyl)amino)phenyl)-1 H-pyrrolo[2,3 -b]pyridin-4 -yl)-1 H-indazol-3 -amine SU1359

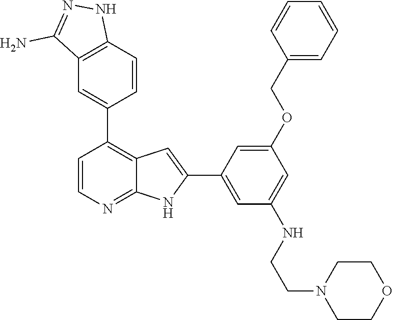 [SMILES: Nc1n[nH]c2ccc(-c3ccnc4[nH]c(-c5cc(NCCN6CCOCC6)cc(OCc6ccccc6)c5)cc34)cc12]

A 2 -5 mL Biotage MW tube was charged with methyl 3 -(benzyloxy)-5 -(4 -chloro-1 H-pyrrolo[2,3 -b]pyridin-2 -yl)-N-(2 -morpholinoethyl)aniline (100 mg, 0.3 mmol), 5 -(4,4,5,5 -tetramethyl-1,3,2 -dioxaborolan-2 -yl)-1 H-indazol-3 -amine (103 mg, 0.4 mmol, 1.3 eq.), [1,1 ′-bis(di-tert-butylphosphino)ferrocene]dichloropalladium(II) catalyst (9.8 mg, 0.015 mmol, 5 mol %) and dioxane (1.8 mL). The resulting suspension was purged with nitrogen for 5 minutes before adding 1 M aq. K₃ PO₄ (1.8 mL). The reaction mixture was heated to 110° C. for 16 hrs. The reaction mixture was then allowed to cool to room temperature, diluted with water (10 mL) and extracted with EtOAc (3×20 mL). The combined organic fractions were then dried over anhydrous magnesium sulfate, filtered and the solvent removed under reduced pressure. The crude product was then purified by HPLC to give the desired product as a pale yellow solid (21 mg). ¹H NMR (500 MHz, DMSO-D₆ ) δ 3.31 (m, 6 H), 3.50 (m, 6 H), 5.11 (s, 2 H), 6.28 (s, 1 H), 6.81 (s, 1 H), 7.00 (s, 1 H), 7.10 (d, J=1.7 Hz, 1 H), 7.21 (d, J=4.9 Hz, 1 H), 7.32 -7.49 (m, 6 H), 7.77 (d, J=8.4 Hz, 1 H), 8.28 (m, 2 H), 12.21 (s, 1 H). LRMS: Calculated for C₃₃H₃₃N₇O₂ 559.27 ; Found:560.40 (M+H)⁺.

### Route to SU1353

### 2 -(2 -(Benzyloxyphenyl)-4 -chloro-1 H-pyrrolo[2,3 -b]pyridine

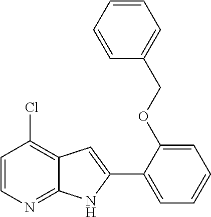 [SMILES: Clc1ccnc2[nH]c(-c3ccccc3OCc3ccccc3)cc12]

A suspension of 4 -chloro-2 -iodo-7 -azaindole (0.343 g, 1.23 mmol), (2 -(benzyloxy)phenyl)boronic acid (0.347 g, 1.52 mmol), K₂ CO₃ (0.483 g, 3.49 mmol) and Bis(triphenylphosphine) palladium(II) chloride (0.074 g, 0.105 mmol) in 1,4 -dioxane (3 mL) and water (2 mL) was degassed under nitrogen. The reaction mixture was heated to 100° C. for 20 h. The reaction mixture was cooled to room temperature and extracted into EtOAc, washed with water, aqueous saturated NaHCO₃ and brine. Organics were concentrated under reduced pressure and the resulting solid triturated with hexane and Et₂O to afford the title compound as an light solid (0.378 g, 1.13 mmol, 92 %). ¹H NMR (DMSO-d₆ ): δ 5.32 (s, 2 H), 7.06 (s, 1 H), 7.09 (t, J=7.5 Hz, 1 H), 7.17 (d, J=5.0 Hz, 1 H), 7.28 (d, J=8.5 Hz, 1 H), 7.36 (m, 2 H), 7.42 (t, J=7.0 Hz, 1 H), 7.54 (d, J=7.5 Hz, 1 H), 7.92 (dd, J=2.0, 8.8 Hz, 1 H), 8.18 (d, J=7.5 Hz, 1 H), 12.19 (br s, 1 H).

### 5 -(2 -(2 -(Benzyloxy)phenyl)-1 H-pyrrolo[2,3 -b]pyridin-4 -yl)-1 H-indazol-3 -amine SU1353

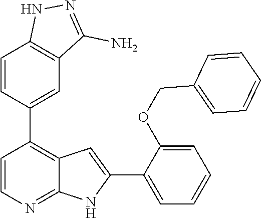 [SMILES: Nc1n[nH]c2ccc(-c3ccnc4[nH]c(-c5ccccc5OCc5ccccc5)cc34)cc12]

To a suspension of 2 -(2 -(benzyloxyphenyl)-4 -chloro-1 H-pyrrolo[2,3 -b]pyridine (0.143 g, 0.43 mmol), 5 -(4,4,5,5 -tetramethyl-1,3,2 -dioxaborolan-2 -yl)-1 H-indazol-3 -amine (0.142 g, 0.55 mmol) and [1,1 ′-bis(di-tert-butylphosphino)ferrocene]dichloropalladium(II) catalyst (0.031 g, 0.048 mmol) in EtOH (3.0 mL, degassed under nitrogen) was added K₃ PO₄ (1 M, 1.2 mL) and the reaction mixture was heated to 120° C. for 20 h. The reaction mixture was cooled to room temperature, diluted with EtOAc, washed with water and brine. The organics were concentrated under reduced pressure and purified by column chromatography (100 % EtOAc—10 % MeOH/EtOAc) and trituration with Et₂O to afford the title compound as an off-white solid (0.053 g, 1.23 mmol, 29 %), contains one molecule of EtOAc. ¹H NMR (DMSO-d₆ ): δ 1.18 (t, J=7.0 Hz, 1 H), 1.99 (s, 3 H), 4.03 (q, J=7.2 Hz, 1 H), 5.24 (s, 2 H), 5.50 (br s, 2 H), 7.11 (m, 1 H), 7.22 (d, J=4.8 Hz, 1 H), 7.27 (m, 6 H), 7.35 (m, 1 H), 7.53 (m, 2 H), 7.57 (dd, J=1.6, 8.8 Hz, 1 H), 7.92 (dd, J=1.6, 7.6 Hz, 1 H), 8.16 (s, 1 H), 8.24 (d, J=4.8 Hz, 1 H), 11.54 (br s, 1 H), 11.85 (br s, 1 H). HRMS: found 432.1814 (M+H)⁺calculated for C₂₇H₂₂N₅O 432.1819

### Route to SU1334

### 2 -(4 -(Benzyloxy)phenyl)-4 -chloro-1 H-pyrrolo[2,3 -b]pyridine

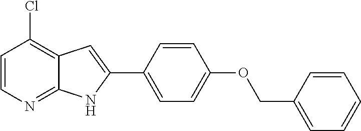 [SMILES: Clc1ccnc2[nH]c(-c3ccc(OCc4ccccc4)cc3)cc12]

A suspension of 4 -chloro-2 -iodo-7 -azaindole (0.364 g, 1.31 mmol), 2 -(4 (benzyloxy)phenyl-4,4,5,5 -tetramethyl-1,3,2 -dioxaborolane (0.35 g, 1.53 mmol), K₂ CO₃ (0.59 g, 3.83 mmol) and Bis(triphenylphosphine) palladium(II) chloride (0.081 g, 0.12 mmol) in 1,4 -dioxane (3 mL) and water (2 mL) was degassed under nitrogen. The reaction mixture was heated to 100° C. for 17 h. The reaction mixture was cooled to room temperature and extracted into EtOAc, washed with water, aqueous saturated NaHCO₃ and brine. Organics were concentrated under reduced pressure and purified by column chromatography (100 % Hexane—3/1 Hexane/EtOAc—1/1 Hexane/EtOAc—100 %). Trituration of the resulting solid with Hexane/Et2O afforded the title compound as an off-white solid (0.189 g, 0.56 mmol, 43 %). ¹H NMR (DMSO-d₆ ): δ 5.19 (s, 2 H), 6.87 (d, J=2.0 Hz, 1 H), 7.12 (d, J=8.5 Hz, 1 H), 7.18 (d, J=5.0 Hz, 1 H), 7.35 (d, J=7.5 Hz, 1 H), 7.41 (t, J=7.5 Hz, 2 H), 7.47 (d, J=7.0 Hz, 2 H), 7.94 (d, J=8.5 Hz, 1 H), 8.13 (d, J=5.5 Hz, 1 H), 12.38 (br s, 1 H).

### 5 -(2 -(4 -Benzyloxy)phenyl)-1 H-pyrrolo[2,3 -b]pyridin-4 -yl)-1 H-indazol-3 -amine SU1334

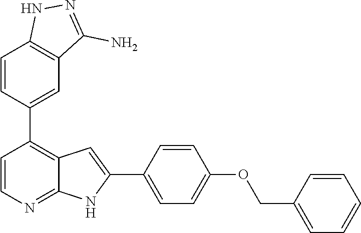 [SMILES: Nc1n[nH]c2ccc(-c3ccnc4[nH]c(-c5ccc(OCc6ccccc6)cc5)cc34)cc12]

To a suspension of 2 -(4 -(benzyloxy)phenyl)-4 -chloro-1 H-pyrrolo[2,3 -b]pyridine (0.112 g, 0.33 mmol), 5 -(4,4,5,5 -tetramethyl-1,3,2 -dioxaborolan-2 -yl)-1 H-indazol-3 -amine (0.11 g, 0.42 mmol) and [1,1 ′-bis(di-tert-butylphosphino)ferrocene]dichloropalladium(II) catalyst (0.023 g, 0.035 mmol) in EtOH (3.0 mL, degassed under nitrogen) was added K₃ PO₄ (1 M, 1.2 mL) and the reaction mixture was heated to 120° C. for 18 h. The reaction mixture was cooled to room temperature and diluted with EtOAc, washed with water, aqueous saturated NaHCO₃ and brine. The organics were concentrated under reduced pressure and the resulting residue and purified by column chromatography (100 % Hexane—1/1 Hexane/EtOAc—100 % EtOAc—10 % MeOH/EtOAc). The resulting solid was triturated with hexane and Et₂O to afford the title compound as a pale yellow solid (0.068 g, 0.16 mmol, 47 %). ¹H NMR (DMSO-d₆ ): δ 5.18 (s, 2 H), 5.53 (br s, 2 H), 7.07 (d, J=2.4 Hz, 1 H), 7.12 (d, J=8.8 Hz, 2 H), 7.18 (d, J=4.8 Hz, 1 H), 7.34 (m, 1 H), 7.41 (m, 3 H), 7.49 (m, 2 H), 7.71 (d, J=1.6, 8.8 Hz, 1 H), 7.92 (d, J=8.8 Hz, 1 H), 8.22 (m, 2 H), 11.53 (br s, 1 H), 12.09 (br s, 1 H). HRMS: found 432.1813 (M+H)'⁰ calculated for C₂₇H₂₂N₅O 432.1819.

### Route to SU1337

### 2 -(5 -(Benzyloxy)pyridin-3 -yl)-4 -chloro-1 H-pyrrolo[2,3 -b]pyridine

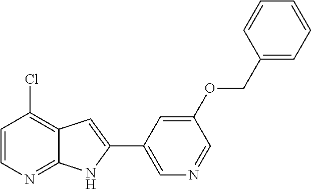 [SMILES: Clc1ccnc2[nH]c(-c3cncc(OCc4ccccc4)c3)cc12]

4 -Chloro-2 -iodo-1 H-pyrrolo[2,3 -b]pyridine (0.082 g, 0.30 mmol), 3 -(benzyloxy)-5 -(4,4,5,5 -tetramethyl-1,3,2 -dioxaborolan-2 -yl)pyridine (0.11 g, 0.353 mmol), [1,1 ′-bis(diphenylphosphino)ferrocene]dichloropalladium II (0.012 g, 0.015 mmol) and a magnetic stirrer bar were added in a 10 -20 mL microwave vial which was sealed with an aluminium crimp cap fitted with a disposable PTFE/silicon septum, and flushed with nitrogen. Then dioxane (4 mL) was added and the resulting mixture was purged with nitrogen for 10 min before adding 1.5 M K₂ CO₃ (aqueous solution, 0.5 mL). The mixture was purged again with nitrogen for 10 min and heated to 100° C. overnight. After cooling, the resulting mixture was filtered through celite and washed with EtOAc (15 mL). The filtrates were concentrated in vacuo to obtain the title compound as brown solid (0.093 g, 0.28 mmol, 93 %). ¹H NMR (400 MHz, DMSO-d₆ ) δ 5.29 (s, 2 H), 7.24 -7.25 (m, 2 H), 7.35 -7.39 (m, 1 H), 7.41 -7.43 (m, 2 H), 7.52 (d, J=7.1 Hz, 2 H), 8.11 (dd, J=2.6, 1.6 Hz, 1 H), 8.22 (d, J=5.2 Hz, 1 H), 8.34 (d, J=2.6 Hz, 1 H), 8.84 (d, J=1.6 Hz, 1 H), 12.67 (s, 1 H). LRMS found 336.1 (M+H)⁺calculated for C₁₉H₁₄N₃OCl 336.1

### 5 -(2 -(5 -(Benzyloxy)pyridin-3 -yl)-1 H-pyrrolo[2,3 -b]pyridin-4 -yl)-1 H-indazol-3 -amine SU1337

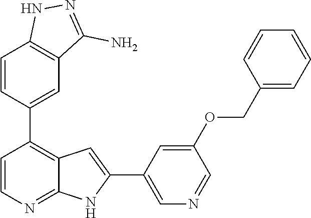 [SMILES: Nc1n[nH]c2ccc(-c3ccnc4[nH]c(-c5cncc(OCc6ccccc6)c5)cc34)cc12]

2 -(5 -(Benzyloxy)pyridin-3 -yl)-4 -chloro-1 H-pyrrolo[2,3 -b]pyridine (0.093 g, 0.28 mmol), 5 -(4,4,5,5 -tetramethyl-1,3,2 -dioxaborolan-2 -yl)-1 H-indazol-3 -amine (0.087 g, 0.33 mmol), [1,1 ′-bis(di-tert-butylphosphino)ferrocene]dichloropalladium(II) catalyst (0.009 g, 0.014 mmol) and a magnetic stirrer bar were added in a 2 -5 mL Biotage microwave vial which was sealed with an aluminium crimp cap fitted with a disposable PTFE/silicon septum, and flushed with nitrogen. Then EtOH (3 mL) was added, and the resulting mixture was purged with nitrogen for 10 min before adding IM K₃ PO₄ (aqueous solution, 0.5 mL). The mixture was purged again with nitrogen for 10 min and heated to 100° C. for 24 hours. After cooling water was added to the reaction mixture and the solid that precipitated was filtered and dried in vacuo. The crude was purified by flash chromatography (SiO₂, 1 % triethylamine in EtOAc—1 % triethylamine in EtOAc:MeOH 90:10 ) to obtain the title compound as beige (0.065 g, 0.15 mmol, 54 %) ¹ H NMR (400 MHz, DMSO-d₆ ) δ 5.28 (s, 2 H), 5.57 (s, 2 H), 7.23 (d, J=5.0 Hz, 1 H), 7.35 -7.47 (m, 5 H), 7.52 (d, J=7.5 Hz, 2 H), 7.74 (d, J=8.6 Hz, 1 H), 8.08 (s, 1 H), 8.24 (s, 1 H), 8.27 -8.34 (m, 2 H), 8.85 (d, J=1.6 Hz, 1 H), 11.58 (s, 1 H), 12.37 (s, 1 H). HRMS found 433.1772 (M+H)⁺calculated for C₂₆H₂₁N₆O 433.1777

### Route to SU1349

### 2 -(2 -(Benzyloxy)pyridin-4 -yl)-4 -chloro-1 H-pyrrolo[2,3 -b]pyridine

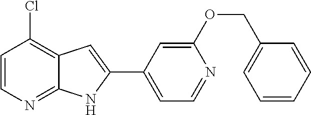 [SMILES: Clc1ccnc2[nH]c(-c3ccnc(OCc4ccccc4)c3)cc12]

4 -Chloro-2 -iodo-1 H-pyrrolo[2,3 -b]pyridine (0.23 g, 0.83 mmol), 2 -(benzyloxy)pyridine-4 -boronic (0.240 g, 0.99 mmol), 1,1 ′-[bis(diphenylphosphino)ferrocene]dichloropalladium II (0.068 g, 0.083 mmol) and a magnetic stirrer bar were added in a 10 -20 mL microwave vial which was sealed with an aluminium crimp cap fitted with a disposable PTFE/silicon septum, and flushed with nitrogen. Then dioxane (8 mL) was added and the resulting mixture was purged with nitrogen for 10 min before adding 1.5 M K₂ CO₃ (aqueous solution, 1.7 mL). The mixture was purged again with nitrogen for 10 min and heated to 100° C. for 24 hours. After cooling, the resulting mixture was filtered through celite and washed with EtOAc (20 mL). The filtrates were concentrated in vacuo to obtain the title compound as brown solid (0.30 g, 0.89 mmol, 90 %). ¹ H NMR (400 MHz, DMSO-d₆ ) δ 5.41 (s, 2 H), 7.26 (d, J=5.3 Hz, 1 H), 7.33 (d, J=1.8 Hz, 1 H), 7.35 (d, J=7.5 Hz, 1 H), 7.38 -7.42 (m, 2 H), 7.52 -7.47 (m, 3 H), 7.63 (d, J=5.7 Hz, 1 H), 8.26 -8.24 (m, 2 H), 12.72 (s, 1 H). LRMS found 336.1 (M+H)⁺calculated for C₁₉H₁₅ ClN₃0 336.1

### 5 -(2 -(2 -(Benzyloxy)pyridin-4 -yl)-1 H-pyrrolo[2,3 -b]pyridin-4 -yl)-1 H-indazol-3 -amine SU1349

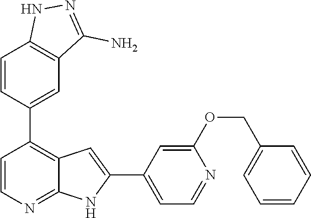 [SMILES: Nc1n[nH]c2ccc(-c3ccnc4[nH]c(-c5ccnc(OCc6ccccc6)c5)cc34)cc12]

2 -(2 -(Benzyloxy)pyridin-4 -yl)-4 -chloro-1 H-pyrrolo[2,3 -b]pyridine (0.140 g, 0.42 mmol), 5 -(4,4,5,5 -tetramethyl-1,3,2 -dioxaborolan-2 -yl)-1 H-indazol-3 -amine (0.118 g, 0.46 mmol), [1,1 ′-bis(di-tert-butylphosphino)ferrocene]dichloropalladium(II) catalyst (0.014 mg, 0.021 mmol) and a magnetic stirrer bar were added in a 2 -5 mL Biotage microwave vial which was sealed with an aluminium crimp cap fitted with a disposable PTFE/silicon septum, and flushed with nitrogen. Then EtOH (4 mL) was added, and the resulting mixture was purged with nitrogen for 10 min before adding 1 M K₃ PO₄ (aqueous solution, 0.83 mL). The mixture was purged again with nitrogen for 10 min and heated to 120° C. for 24 hours. After cooling water was added to the reaction mixture and the solid that precipitated was filtered and dried in vacuo. The crude was purified by flash chromatography (SiO₂, EtOAc-EtOAc:MeOH, 90:10 ) to obtain the title compound as white solid (0.089 g, 0.20 mmol, 49 %). ¹H NMR (400 MHz, DMSO-d₆ ) δ 5.41 (s, 2 H), 5.57 (s, 2 H), 7.25 (d, J=5.0 Hz, 1 H), 7.34 (d, J=7.3 Hz, 1 H), 7.38 -7.42 (m, 3 H), 7.45 -7.53 (m, 4 H), 7.62 (dd, J=5.4, 1.6 Hz, 1 H), 7.73 (dd, J=8.7, 1.6 Hz, 1 H), 8.20 -8.27 (m, 2 H), 8.33 (d, J=5.0 Hz, 1 H), 11.58 (s, 1 H), 12.43 (s, 1 H). HRMS found 433.1769 (M+H)⁺calculated for C₂₆H₂₁N₆O 433.1777

### Route to SU1350

### 2 -(6 -(Benzyloxy)pyridin-3 -yl)-4 -chloro-1 H-pyrrolo[2,3 -b]pyridine

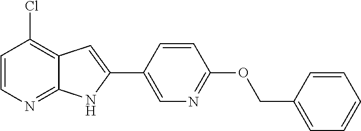 [SMILES: Clc1ccnc2[nH]c(-c3ccc(OCc4ccccc4)nc3)cc12]

4 -Chloro-2 -iodo-1 H-pyrrolo[3 -b]pyridine (0.2 g, 0.90 mmol), -benzyloxy-5 -pyridineboronic acid pinacol ester (0.335 g, 1.08 mmol), [1,1 ′-bis(diphenylphosphino)ferrocene]dichloropalladium II (0.073 g, 0.089 mmol) and a magnetic stirrer bar were added in a 10 -20 mL microwave vial which was sealed with an aluminium crimp cap fitted with a disposable PTFE/silicon septum, and flushed with nitrogen. Then dioxane (8 mL) was added and the resulting mixture was purged with nitrogen for 10 min before adding 1.5 M K₂ CO₃ (aqueous solution, 1.8 mL). The mixture was purged again with nitrogen for 10 min and heated to 100° C. for 24 hours. After cooling, the resulting mixture was filtered through celite and washed with EtOAc (20 mL). The filtrates were concentrated in vacuo and the residue was purified by flash chromatography (SiO₂, hexane:EtOAc, 80:20 - hexane:EtOAc, 30:70 ) to obtain the title compound as white solid (0.134 g, 0.40 mmol, 44 %). ¹H NMR (400 MHz, DMSO-d₆ ) δ 5.42 (s, 2 H), 6.99 -7.05 (m, 2 H), 7.21 (d, J=5.1 Hz, 1 H), 7.35 (d, J=7.1 Hz, 1 H), 7.42 -7.38 (m, 2 H), 7.48 (d, J=7.0 Hz, 2 H), 8.17 (d, J=5.2 Hz, 1 H), 8.33 (dd, J=8.7, 2.3 Hz, 1 H), 8.82 (d, J=2.3 Hz, 1 H), 12.55 (s, 1 H). LRMS found 336.1 (M+H)⁺ calculated for C₁₉H₁₅N₃O 336.1

### 5 -(2 -(6 -(Benzyloxy)pyridin-3 -yl)-1 H-pyrrolo[2,3 -b]pyridin-4 -yl)-1 H-indazol-3 -amine SU1350

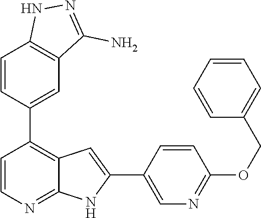 [SMILES: Nc1n[nH]c2ccc(-c3ccnc4[nH]c(-c5ccc(OCc6ccccc6)nc5)cc34)cc12]

2 -(6 -(Benzyloxy)pyridin-3 -yl)-4 -chloro-1 H-pyrrolo[2,3 -b]pyridine (0.100 g, 0.30 mmol), 5 -(4,4,5,5 -tetramethyl-1,3,2 -dioxaborolan-2 -yl)-1 H-indazol-3 -amine (0.092 g, 0.36 mmol), [1,1 ′-bis(di-tert-butylphosphino)ferrocene]dichloropalladium(II) catalyst (0.010 mg, 0.015 mmol) and a magnetic stirrer bar were added in a 2 -5 mL Biotage microwave vial which was sealed with an aluminium crimp cap fitted with a disposable PTFE/silicon septum, and flushed with nitrogen. Then EtOH (4 mL) was added, and the resulting mixture was purged with nitrogen for 10 min before adding 1 M K₃ PO₄ (aqueous solution, 0.60 mL). The mixture was purged again with nitrogen for 10 min and heated to 100° C. for 24 hours. After cooling water was added to the reaction mixture and the solid that precipitated was filtered and dried in vacuo. The crude was purified by flash chromatography (SiO₂, EtOAc-EtOAc:MeOH, 90:10 ) to obtain the title compound as beige solid (0.035 g, 0.82 mmol, 27 %). ¹H NMR (400 MHz, DMSO-d₆ ) δ 5.42 (s, 2 H), 5.56 (s, 2 H), 7.01 (d, J=8.9 Hz, 1 H), 7.19 -7.21 (m, 2 H), 7.34 (d, J=7.4 Hz, 1 H), 7.36 -7.42 (m, 3 H), 7.48 (m, 2 H), 7.72 (d, J=8.9 Hz, 1 H), 8.23 (s, 1 H), 8.25 (d, J=5.0 Hz, 1 H), 8.32 (dd, J=8.4, 2.3 Hz, 1 H), 8.81 (d, J=2.3 Hz, 1 H), 11.57 (s, 1 H), 12.27 (s, 1 H). HRMS found 433.1771 (M+H)⁺calculated for C₂₆H₂₁N₆O 433.1777

### Route to SU1602

### 4 -Chloro-1 -(methoxymethyl)-1 H pyrrolo[2,3 -b]pyridine

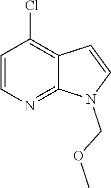

To a solution of 4 -chloro-1 H-pyrrolo[2,3 -b]pyridine (5.0 g, 32.77 mmol) and potassium carbonate (6.793 g, 49.15 mmol) in DMF (20 mL) cooled to 0° C. was added methoxymethyl chloride (MOMCl) (3.1659 g, 39.33 mmol). The resulting mixture was slowly allowed to warm up to room temperature and stirred for 18 hours. The reaction was then quenched by pouring into a mixture of ice/water and extracted with EtOAc (3×50 mL). The combined organic layers were washed with brine (3×20 mL) and purified by flash column chromatography on silica gel eluting with a gradient of petroleum ether: EtOAc (50 to 100 %) to afford the target compound as a cream coloured solid (4.62 g, 72 %). ¹H NMR (500 MHz, DMSO-d₆ ) δ 8.26 (d, J=5.2 Hz, 1 H), 7.78 (d, J=3.6 Hz, 1 H), 7.29 (d, J=5.2 Hz, 1 H), 6.61 (d, J=3.6 Hz, 1 H), 5.62 (s, 2 H), 3.22 (s, 3 H). LRMS (ESI) m/z [M]⁺ For C₉H₉ ClN₂0 Molecular Weight: 196.6340 found 197.3.

### 4 -Chloro-2 -iodo-1 -(methoxymethyl)-1 H-pyrrolo[2,3 -b]pyridine

To a solution of 4 -chloro-1 -(methoxymethyl)-1 H pyrrolo[2,3 -b]pyridine (1.422 g, 7.253 mmol) in anhydrous THF cooled to −78° C. under an atmosphere of argon was added n-butyllithium (8.7 mL, 8.70 mmol, 1 M solution in THF) slowly and the resulting reaction mixture allowed to age for 30 minutes at 0° C. The reaction was then cooled back to −78° C., iodine (1.0125 g, 7.978 mmol) was slowly added as a solution in THF (10 mL) and the reaction allowed to warm up to room temperature and stirred for a further 2 hours. The reaction was then quenched by pouring into a mixture of ice/H₂O and extracted with EtOAc (3×50 mL). The combined organic phase was then washed with saturated ammonium chloride (20 mL), H₂O (20 mL) and brine (20 mL), concentrated under reduced pressure and purified by flash column chromatography on silica gel eluting with a gradient of petroleum ether: EtOAc (20 to 100 %) to afford the target compound as a light brown solid (1.0 g, 43 %). ¹H NMR (500 MHz, DMSO-d₆ ) δ 8.21 (d, J=5.2 Hz, 1 H), 7.29 (d, J=5.2 Hz, 1 H), 7.01 (s, 1 H), 5.61 (s, 2 H), 3.23 (s, 3 H). LRMS (ESI) m/z [M]⁺ For C₉H₈ ClIN₂O Molecular Weight: 322.5305 found 323.0

### 4 -Chloro-1 -(methoxymethyl)-2 -(4,4,5,5 -tetramethyl-1,3,2 -dioxaborolan-2 -yl)-1 H-pyrrolo[2,3 -b]pyridine

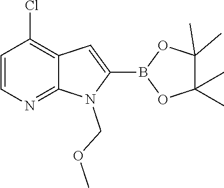 [SMILES: COCn1c(B2OC(C)(C)C(C)(C)O2)cc2c(Cl)ccnc21]

To a freshly degassed solution of 4 -chloro-2 -iodo-1 -(methoxymethyl)-1 H-pyrrolo[2,3 -b]pyridine(1.0 g, 3.106 mmol) in a mixture of dioxane and triethylamine(triethylamine) (4:1, 20 mL) under a blanket of argon was added (BPin)₂ (0.477 g, 0.541 mL, 3.727 mmol) and Tetrakis(triphenylphosphine)palladium(0 ) (0.108 g, 0.0931 mmol) and the resultant mixture stirred at 80° C. overnight. The reaction was then concentrated under reduced pressure and and purified by flash column chromatography on silica gel eluting with a gradient (0 to 50 %) EtOAc in petroleum ether to afford the target compound as a light brown solid (0.5 g, 50 %). ¹ H NMR (500 MHz, DMSO-d₆ ) δ 8.35 (d, J=5.1 Hz, 1 H), 7.34 (d, J=5.1 Hz, 1 H), 7.05 (s, 1 H), 5.79 (s, 2 H), 3.19 (s, 3 H), 1.34 (s, 12 H). For C₁₅H₂₀BClN₂O₃ Molecular Weight: 322.5960 found 323.3.

### 4 -(Benzyloxy)-2 -bromopyridine

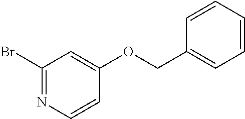 [SMILES: Brc1cc(OCc2ccccc2)ccn1]

To a mixture of 2 -bromopyridin-4 -ol (0.376 g, 2.16 mmol) and cesium carbonate (1.037 g, 3.182 mmol in DMF (5 mL) under a blanket argon cooled to 0° C. was added benzyl bromide (0.436 g, 0.303 mL) and the resulting mixture allowed to warm up to room temperature and stirred for a further 4 hours. The reaction was then quenched by the pouring over ice/water (20 mL) and extractedcesium carbonate with EtOAc (3×50 mL). The combined organic phase was washed with brine (3×20 mL), concentrated under reduced pressure and purified by flash column chromatography on silica gel eluting with a gradient from (0 to 50 %) EtOAc in petroleum ether to afford the target compound as a light brown solid (0.47 g, 82 %).¹ H NMR (400 MHz, DMSO-d₆ ) δ 8.20 (d, J=5.8 Hz, 1 H), 7.54 -7.34 (m, 6 H), 7.11 (dd, J=5.8, 2.3 Hz, 1 H), 5.24 (s, 2 H).

LRMS (ESI) m/z [M]⁺ For C₁₂H₁₀BrNO Molecular Weight: 264.1220 found 264.0, 265.0. 2 -(4 -(Benzyloxy)pyridin-2 -yl)-4 -chloro-1 -(methoxymethyl)-1 H-pyrrolo[2,3 -b]pyridine

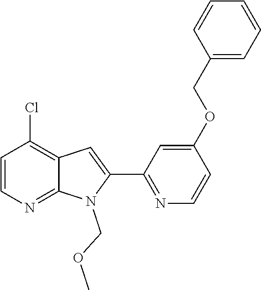 [SMILES: COCn1c(-c2cc(OCc3ccccc3)ccn2)cc2c(Cl)ccnc21]

To a mixture of 4 -chloro-1 -(methoxymethyl)-2 -(4,4,5,5 -tetramethyl-1,3,2 -dioxaborolan-2 -yl)-1 H-pyrrolo[2,3 -b]pyridine (0.366, 1.34 mmol), 4 -(benzyloxy)-2 -bromopyridine (0.328 g, 1.247 mmol), potassium hydroxide (KOH) (0.317 g, 5.66 mmol) and [1,1 ′-bis(diphenylphosphino)ferrocene]dichloropalladium(II) catalyst (0.0414 g, 0.0566 mmol) in a sealed tube under an atmosphere of argon was added a freshly degassed mixture of dioxane:water (18:2, 10 mL) and the resulting mixture stirred at 110° C. overnight. The reaction was then cooled and diluted with EtOAc and washed with water (3×20 mL). The organic layer was concentrated under reduced pressure and purified by flash column chromatography on silica gel eluting with a gradient (0 to 50 %) EtOAc in petroleum ether +1 % triethylamine to afford the target compound as a brown solid (0.376 g, 99 %).¹ H NMR (500 MHz, DMSO-d₆ ) δ 8.54 (d, J=5.7 Hz, 1 H), 8.34 (d, J=5.2 Hz, 1 H), 7.73 (d, J=2.4 Hz, 1 H), 7.54 -7.48 (m, 2 H), 7.44 (td, J=7.3, 6.3, 1.5 Hz, 2 H), 7.38 (dd, J=8.0, 6.2 Hz, 2 H), 7.25 (s, 1 H), 7.10 (dd, J=5.8, 2.4 Hz, 1 H), 6.21 (s, 2 H), 5.33 (s, 2 H), 3.08 (s, 3 H). LRMS (ESI) m/z [M]⁺ For C₂₁H₁₈ ClN₃02 Molecular Weight: 379.8440 found 380.3

### 5 -(2 -(4 -(Benzyloxy)pyridin-2 -yl)-1 H-pyrrolo[2,3 -b]pyridin-4 -yl)-1 H-indazol-3 -amine SU1602

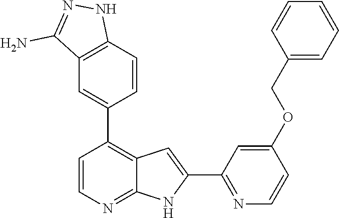 [SMILES: Nc1n[nH]c2ccc(-c3ccnc4[nH]c(-c5cc(OCc6ccccc6)ccn5)cc34)cc12]

To a mixture of 2 -(4 -(benzyloxy)pyridin-2 -yl)-4 -chloro-1 -(methoxymethyl)-1 H-pyrrolo[2,3 -b]pyridine (0.09 g, 2.373 mmol), 5 -(4,4,5,5 -tetramethyl-1,3,2 -dioxaborolan-2 -yl)-1 H-indazol-3 -amine (0.0676 g, 0.261 mmol), [1,1 ′-bis(di-tert-butylphosphino)ferrocene]dichloropalladium(II) catalyst (0.0154 g, 0.0236 mmol) and cesium carbonate (0.232 g, 0.712 mmol) in a sealed tube flushed with argon was added a freshly degassed mixture of dioxane:water (18:2, 10 mL) and the resulting mixture stirred at 110° C. overnight. The reaction was then cooled and diluted with EtOAc and washed with water (3×20 mL). The organic layer was concentrated under reduced pressure and purified by flash column chromatography on silica gel eluting with a gradient (0 to 100 %) EtOAc in petroleum ether+1 % triethylamine to afford 5 -(2 -(4 -(benzyloxy)pyridin-2 -yl)-1 -(methoxymethyl)-1 H-pyrrolo[2,3 -b]pyridin-4 -yl)-1 H-indazol-3 -amine as an orange solid (0.063 g, 56 %). ¹H NMR (500 MHz, DMSO-d₆ ) δ 11.59 (s, 1 H), 8.54 (d, J=5.7 Hz, 1 H), 8.41 (d, J=5.0 Hz, 1 H), 8.27 (d, J=1.7 Hz, 1 H), 7.74 (dd, J=8.6, 1.7 Hz, 1 H), 7.68 (d, J=2.5 Hz, 1 H), 7.54 -7.49 (m, 2 H), 7.42 (dd, J=8.2, 6.3 Hz, 3 H), 7.39 -7.33 (m, 3 H), 7.09 (dd, J=5.7, 2.4 Hz, 1 H), 6.24 (s, 2 H), 5.56 (s, 2 H), 5.30 (s, 2 H), 3.11 (s, 3 H).

The obtained compound was then taken up in a mixture of concentrated HCl:MeOH (4:1, 5 mL) and stirred at 85° C. for 4 hours, cooled, concentrated under reduced pressure and purified by HPLC to afford the target compound as an orange solid (0.018 g, 31 %) ¹H NMR (500 MHz, DMSO-d₆ ) δ 12.60 (s, 2 H), 8.57 (d, J=5.9 Hz, 1 H), 8.41 (d, J=5.1 Hz, 1 H), 8.35 (d, J=1.5 Hz, 1 H), 7.95 (d, J=2.4 Hz, 1 H), 7.85 (dd, J=8.7, 1.7 Hz, 1 H), 7.63 (s, 1 H), 7.57 -7.51 (m, 4 H), 7.48 -7.44 (m, 2 H), 7.43 -7.38 (m, 1 H), 7.32 (d, J=5.0 Hz, 1 H), 7.17 (dd, J=6.2, 2.4 Hz, 1 H), 5.37 (s, 2 H). LRMS (ESI) m/z for Chemical Formula: C₂₆H₂₀N₆O Molecular Weight: 432.4870 found 433.3 (M+H)⁺.

### Route to SU1627

### 4 -Chloro-2 -iodo-1 H-pyrrolo[2,3 -b]pyridine

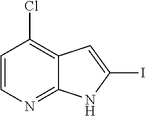 [SMILES: Clc1ccnc2[nH]c(I)cc12]

To a solution of 4 -chloro-2 -iodo-1 -(methoxymethyl)-1 H-pyrrolo[2,3 -b]pyridine (3.39 g, 10.53 mmol) in anhydrous dichloromethane (30 mL) under a stream of argon and cooled to 0° C. was added a solution of bromo-chatecolborane (4.19 g, 21.056 mmol) in dichloromethane (10 mL) slowly and the resulting mixture allowed to warm up to room temperature and stirred for 4 hrs. The reaction was quenched by the slow addition of potassium acetate (5.0 g, 50.942 mmol) in ethanol (10 mL) and stirred for a further 24 hours at room temperature. The solvent was removed under reduced pressure and triturated with water for 30 minutes at 0° C. The resultant suspension was filtered off and washed with saturated sodium bicarbonate followed by water to afford the target compound as a cream coloured solid (2.99 g, 100 %). ¹H NMR (400 MHz, DMSO-d₆ ) δ 12.62 (s, 1 H), 8.11 (d, J=5.2 Hz, 1 H), 7.17 (d, J=5.2 Hz, 1 H), 6.76 (s, 1 H).

LRMS (ESI) m/z [M]⁺ For C₇H₄ ClIN₂ Molecular Weight: 278.4775 found 279.3

### tert-Butyl 4 -chloro-1 H-pyrrolo[2,3 -b]pyridine-I-carboxylate

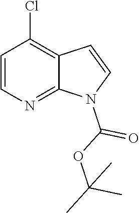 [SMILES: CC(C)(C)OC(=O)n1ccc2c(Cl)ccnc21]

To a solution of 4 -chloro-1 H-pyrrolo[2,3 -b]pyridine (9.6 g, 62.92 mmol) in DMF (10 mL), 4 -dimethylaminopyridine (DMAP) (11.59 g, 94.85 mmol) and di-tert-butyl dicarbonate (BOC20 ) (20.70 g, 21.81 mL, 94.85 mmol) were added under a stream of argon and the resulting solution stirred a room temperature overnight. The reaction was then quenched by pouring into ice/H₂O (60 mL) and extracted with EtOAc. The combined organic layers were washed with brine (3×20 mL) and concentrated under reduced pressure. Purification by flash column chromatography on silica gel elution with a gradient from 0 to 50 % EtOAc in petroleum ether afforded the desired product as a cream colored solid (15.86 g, 99 %). ¹H NMR (500 MHz, DMSO-d₆ ) δ 8.36 (d, J=5.2 Hz, 1 H), 7.89 (d, J=4.0 Hz, 1 H), 7.44 (dd, J=5.2, 1.1 Hz, 1 H), 6.74 (dd, J=4.1, 1.2 Hz, 1 H), 1.62 (s, 9 H). LRMS (ESI) m/z [M]⁺ For C₁₂H₁₃ ClN₂O₂ Molecular Weight: 252.6980 found 253.3 .

### tert-Butyl 4 -chloro-2 -iodo-1 H-pyrrolo[2,3 -b]pyridine-I-carboxylate

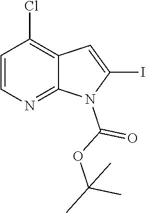 [SMILES: CC(C)(C)OC(=O)n1c(I)cc2c(Cl)ccnc21]

Method 1 : to a solution of 4 -chloro-2 -iodo-1 H-pyrrolo[2,3 -b]pyridine (2.0 g, 7.19 mmol) DMF(10 mL), DMAP (1.318 g, 10.795 mmol) and BOC₂O (1.884 g, 8.63 mmol) were added under a stream of argon and the resulting solution stirred a room temperature overnight. The reaction was then quenched by pouring into ice/H₂O 60 mL and extracted with EtOAc. The combined organic layers were washed with brine (3×20 mL) and concentrated under reduced pressure. Purification by flash column chromatography on silica gel elution with a gradient from 0 to 50 % EtOAc in petroleum ether afforded the desired product as a cream colored solid in quantitative yield. ⁴ H NMR (400 MHz, DMSO-d₆ ) δ 8.24 (d, J=5.2 Hz, 1 H), 7.34 (d, J=5.3 Hz, 1 H), 7.07 (s, 1 H), 1.65 (s, 9 H). LRMS (ESI) m/z [M]⁺ For C₁₂H₁₂ ClIN₂O₂ Molecular Weight: 378.5945 found 379.4

Method 2 : to a solution of tert-butyl 4 -chloro-1 H-pyrrolo[2,3 -b]pyridine-1 -carboxylate (4.65 g, 18.44 mmol) in anhydrous THF (40 mL) cooled to −78° C. under an atmosphere of argon was added LDA 18.44 mL, 36.88 mmol, 2 M solution in THF) slowly and the resulting reaction mixture allowed to age for 30 minutes at 0° C. The reaction was then cooled back to −78° C. and Iodine (Iodine 3.511 g, 27.67 mmol) slowly added as a solution in THF (10 mL) and the reaction allowed to warm up to room temperature and stirred for a further 2 hours. The reaction was then quenched by pouring into a mixture of ice/water and extracted with EtOAc (3×50 mL). The combined organic phase was then washed with saturated Ammonium chloride (20 mL), H₂O (20 mL) and brine (20 mL), concentrated under reduced pressure and purified by flash column chromatography on silica gel eluting with a gradient of petroleum ether: EtOAc (0 to 50 %) to afford the target compound as a light brown solid (4.54 g, 65 %). ¹H NMR (500 MHz, DMSO-d₆ ) δ 8.19 (t, J=1.0 Hz, 1 H), 7.53 (t, J=1.4 Hz, 1 H), 7.05 (dd, J=1.7, 0.9 Hz, 1 H), 1.58 (s, 9 H). LRMS (ESI) m/z [M]” For Chemical Formula: C₁₂H₁₂ ClIN₂O₂ Molecular Weight: 378.5945 found 379.4

### 5 -(((4 -Bromopyridin-2 -yl)oxy)methyl)pyrimidine

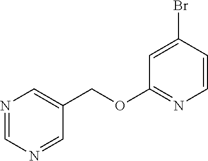 [SMILES: Brc1ccnc(OCc2cncnc2)c1]

To 4 -bromo-2 -fluoropyridine (0.2 g, 1.136 mmol) in anhydrous THF cooled to 0° C. potassium tert-butoxide (0.58 g g, 1.36 mmol in anhydrous THF (10 mL) was added under a blanket of argon. Pyrimidin-5 -ylmethanol (0.15017 g, 1.36 mmol) was then added and the resulting mixture allowed to warm up to room temperature and stirred for a further 4 hours. The reaction was then quenched by the pouring over ice/water (20 mL) and extracted with EtOAc (3×50mL). The combined organic phase was washed with brine (3×20 mL), concentrated under reduced pressure and purified by flash column chromatography on silica gel eluting with a gradient from (0 to 50 %) EtOAc in petroleum ether to afford the target compound as a light brown oil in quantitative yield. ¹H NMR (400 MHz, DMSO-d₆ ) δ 9.17 (s, 1 H), 8.93 (s, 2 H), 8.12 (d, J=5.5 Hz, 1 H), 7.29 (dd, J=5.5, 1.7 Hz, 1 H), 7.26 (d, J=1.7 Hz, 1 H), 5.43 (s, 2 H).LRMS (ESI) m/z [M]⁺ C₁₀H₈BrN₃0 Molecular Weight: 266.0980 found 266.0, 267.0 5 -(((4 -(4,4,5,5 -Tetramethyl-1,3,2 -dioxaborolan-2 -yi)pyridin-2 -yl)oxy)methyl)pyrimidine

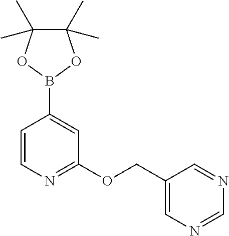 [SMILES: CC1(C)OB(c2ccnc(OCc3cncnc3)c2)OC1(C)C]

To a mixture of 5 -(((4 -bromopyridin-2 -yl)oxy)methyl)pyrimidine (0.15 g, 0.56 mmol), [1,1 ′-bis(diphenylphosphino)ferrocene]dichloropalladium(II) catalyst (0.0412 g, 0.056 mmol), potassium acetate (KOAc) (0.166 g, 1.69 mmol), and bis(pinacolato)diboron ((Bpin)₂ ) (0.186 g, 0.73 mmol) in a sealed tube under an atmosphere of argon was added a freshly degassed mixture of Dioaxane:water (18:2, 10 mL) and the resulting mixture stirred at 110° C. overnight. The reaction was then cooled and diluted with EtOAc (30 mL) and washed with water (3×20 mL). The organic layer was concentrated under reduced pressure and purified by flash column chromatography on silica gel eluting with a gradient (0 to 50 %) EtOAc in petroleum ether to afford the target compound as a brown solid in quantitative yield. ¹H NMR (400 MHz, DMSO-d₆ ) δ 9.17 (s, 1 H), 8.93 (s, 2 H), 8.12 (dd, J=5.5, 0.5 Hz, 1 H), 7.29 (dd, J=5.5, 1.7 Hz, 1 H), 7.26 (dd, J=1.7, 0.6 Hz, 1 H), 5.43 (d, J=0.6 Hz, 2 H), 1.41 (s, 12 H). LRMS (ESI) m/z [M]⁺ .For C₁₆H₂₁BN₄O₂ Molecular Weight: 312.1800 found 313.1

### tert-Butyl-4 -chloro-2 -(2 -(pyrimidin-5 -ylmethoxy)pyridin-4 -yl)-1 H-pyrrolo[2,3 -b]pyridine-1 -carboxylate

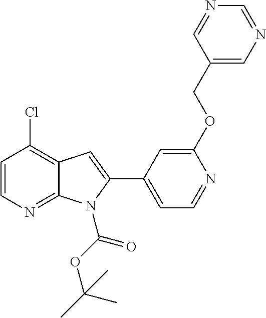 [SMILES: CC(C)(C)OC(=O)n1c(-c2ccnc(OCc3cncnc3)c2)cc2c(Cl)ccnc21]

To a mixture of tert-butyl 4 -chloro-2 -iodo-1 H-pyrrolo[2,3 -b]pyridine-1 -carboxylate (0.3785 g, 0.945 mmol), 5 -(((4 -bromopyridin-2 -yl)oxy)methyl)pyrimidine (0.302 g, 1.135 mmol), potassium hydroxide (KOH) (0.158 g, 2.83 mmol) and [1,1 ′-bis(diphenylphosphino)ferrocene]dichloropalladium(II) catalyst (0.0345 g, 0.04725 mmol in a sealed tube under an atmosphere of argon was added a freshly degassed mixture of dioxane:water (18:2, 10 mL) and the resulting mixture stirred at 110° C. overnight. The reaction was then cooled and diluted with EtOAc and washed with water (3×20 mL). The organic layer was concentrated under reduced pressure and purified by flash column chromatography on silica gel eluting with a gradient (0 to 50 %) EtOAc in petroleum ether+1 % triethylamine to afford the target compound as a crude product (0.14 g, 34 %) that was progressed directly to the next stage.

### 5 -(2 -(2 -(Pyrimidin-5 -ylmethoxy)pyridin-4 -yl)-1 H-pyrrolo[2,3 -b]pyridin-4 -yl)-1 H-indazol-3 -amine SU1627

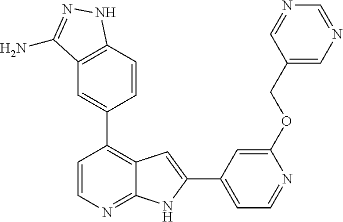 [SMILES: Nc1n[nH]c2ccc(-c3ccnc4[nH]c(-c5ccnc(OCc6cncnc6)c5)cc34)cc12]

To tert-butyl 4 -chloro-2 -(2 -(pyrimidin-5 -ylmethoxy)pyridin-4 -yl)-1 H-pyrrolo[2,3 -b]pyridine-1 -carboxylate (0.135 g, 0.3088 mmol), 5 -(4,4,5,5 -tetramethyl-1,3,2 -dioxaborolan-2 -yl)-1 H-indazol-3 -amine (0.096 g, 0.3706 mmol), cesium carbonate (0.3018 g, 0.9264 mmol), and [1,1 ′-bis(di-tert-butylphosphino)ferrocene]dichloropalladium(II) catalyst (0.020 g, 0.03088 mmol) in a sealed tube under an atmosphere of argon was added a freshly degassed mixture of dioxane:water (18:2, 15 mL) and the resulting mixture stirred at 110° C. overnight. The reaction was then cooled and diluted with EtOAc (40 mL) and washed with water (3×20 mL). The organic layer was concentrated under reduced pressure. The crude product was dissolved in THF (20 mL) and TBAF (5 mL, 1 M solution in THF) added and refluxed for 8 hours, cooled, concentrated and purified by HPLC to afford the title compound as a bright yellow solid (0.005 g, 4 %) ¹H NMR (400 MHz, DMSO-d₆ ) 6 12.47 (d, J=2.1 Hz, 1 H), 9.18 (s, 1 H), 8.96 (s, 1 H), 8.36 (d, J=5.0 Hz, 1 H), 8.30 (d, J=1.5 Hz, 1 H), 8.26 (d, J=5.5 Hz, 1 H), 7.80 (dd, J=8.7, 1.7 Hz, 1 H), 7.65 (dd, J=5.5, 1.5 Hz, 1 H), 7.54 (d, J=1.4 Hz, 1 H), 7.49 (d, J=2.1 Hz, 1 H), 7.46 (d, J=8.7 Hz, 1 H), 7.27 (d, J=5.0 Hz, 1 H), 5.48 (s, 2 H). LRMS (ESI) m/z for C₂₄H₁₈N₈₀ Molecular Weight: 434.4630 found 435.2 (M+H)⁺.

### Route to SU1628

### 4 -Bromo-2 -(pyridin-4 -ylmethoxy)pyridine

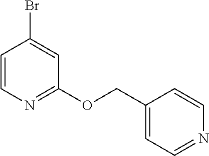 [SMILES: Brc1ccnc(OCc2ccncc2)c1]

To 4 -bromo-2 -fluoropyridine (0.2 g, 1.136 mmol) in anhydrous THF cooled to 0° C., potassium tert-butoxide (0.58 g g, 1.36 mmol) in anhydrous THF (10 mL) was added under a blanket of argon. Pyridin-4 -ylmethanol (0.1488 g, 1.36 mmol) was then added and the resulting mixture allowed to warm up to room temperature and stirred for a further 4 hours. The reaction was then quenched by pouring over ice/water (20 mL) and extracted with EtOAc (3×50 mL). The combined organic phase was washed with brine (3×20 mL), concentrated under reduced pressure, and purified by flash column chromatography on silica gel eluting with a gradient from (0 to 50 %) EtOAc in petroleum ether to afford the target compound as a light brown oil in quantitative yield. ¹H NMR (500 MHz, DMSO-d₆ ) δ 8.56 (ddd, J=4.8, 1.8, 0.9 Hz, 1 H), 8.07 (d, J=5.4 Hz, 1 H), 7.81 (td, J=7.7, 1.8 Hz, 1 H), 7.45 (dt, J=7.9, 1.0 Hz, 1 H), 7.33 (ddd, J=7.6, 4.9, 1.2 Hz, 1 H), 7.29 -7.24 (m, 2 H), 5.44 (s, 2 H). LRMS (ESI) m/z [M]⁺ C₁₁H₉BrN₂O Molecular Weight: 265.1100 found 265.0, 266.0

### 2 -(Pyridin-4 -ylmethoxy)-4 -(4,4,5,5 -tetramethyl-1,3,2 -dioxaborolan-2 -yl)pyridine

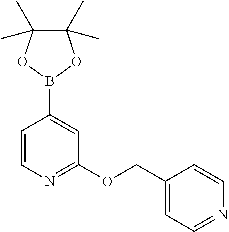 [SMILES: CC1(C)OB(c2ccnc(OCc3ccncc3)c2)OC1(C)C]

To a mixture of 4 -bromo-2 -(pyridin-4 -ylmethoxy)pyridine (1.69 g, 6.3747 mmol), [1,1 ′-bis(diphenylphosphino)ferrocene]dichloropalladium(II) catalyst (0.238 g, 0.325 mmol), potassium acetate (KOAc) (1.918 g, 19.541 mmol), and bis(pinacolato)diboron ((Bpin)₂ ) (2.15 g, 8.466 mmol) in a sealed tube under an atmosphere of argon was added a freshly degassed mixture of dioxane:water (18:2, 10 mL) and the resulting mixture stirred at 110° C. overnight. The reaction was then cooled and diluted with EtOAc (30 mL) and washed with water (3×20 mL). The organic layer was concentrated under reduced pressure and purified by flash column chromatography on silica gel eluting with a gradient (0 to 50 %) EtOAc in petroleum ether to afford the target compound as a brown solid (0.98 g, 49 %). ¹H NMR (500 MHz, DMSO-d₆ ) δ 8.64 (s, 2 H), 8.18 (dd, J=5.0, 0.8 Hz, 1 H), 7.45 (s, 2 H), 7.17 (dd, J=5.0, 0.8 Hz, 1 H), 7.11 (s, 1 H), 5.43 (s, 2 H), 1.30 (s, 12 H). LRMS (ESI) m/z [M]⁺ For C₁₇H₂₁BN₂O₃ Molecular Weight: 312.1760 found 313.0

### tert-Butyl 4 -chloro-2 -(2 -(pyridin-4 -ylmethoxy)pyridin-4 -yl)-1 H-pyrrolo[2,3 -b]pyridine-1 -carboxylate

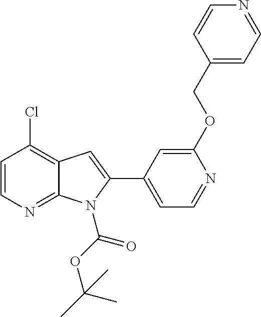 [SMILES: CC(C)(C)OC(=O)n1c(-c2ccnc(OCc3ccncc3)c2)cc2c(Cl)ccnc21]

To a mixture of tert-butyl 4 -chloro-2 -iodo-1 H-pyrrolo[2,3 -b]pyridine-1 -carboxylate (1.0 g, 2.64 mmol), 2 -(pyridin-4 -ylmethoxy)-4 -(4,4,5,5 -tetramethyl-1,3,2 -dioxaborolan-2 -yl)pyridine (0.98 g, 3.139 mmol) bis(triphenylphosphine)palladium(II) dichloride (0.0926 g, 0.132 mmol) and cesium carbonate (2.58 g, 7.9 mmol) in a sealed tube under an atmosphere of argon was added a freshly degassed mixture of dioxane:water (18:2, 10 mL) and the resulting mixture stirred at 80° C. overnight. The reaction was then cooled and diluted with EtOAc and washed with water (3×20 mL). The organic layer was concentrated under reduced pressure and purified by flash column chromatography on silica gel eluting with a gradient (0 to 50 %) EtOAc in petroleum ether+1 % triethylamine to afford the target compound as a crude product (0.81 g, 70 %) that was progressed directly to the next stage.

### 5 -(2 -(2 -(Pyridin-4 -ylmethoxy)pyridin-4 -yl)-1 H-pyrrolo[2,3 -b]pyridin-4 -yl)-1 H-indazol-3 -amine SU1628

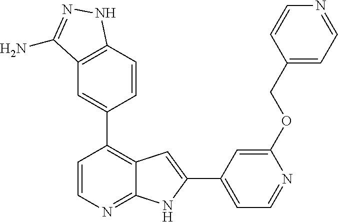 [SMILES: Nc1n[nH]c2ccc(-c3ccnc4[nH]c(-c5ccnc(OCc6ccncc6)c5)cc34)cc12]

To tert-butyl 4 -chloro-2 -(2 -(pyridin-4 -ylmethoxy)pyridin-4 -yl)-1 H-pyrrolo[2,3 -b]pyridine-1 -carboxylate (0.81 g, 1.854 mmol), (3 -cyano-4 -fluorophenyl)boronic acid (0.476 g, 2.884 mmol), cesium carbonate (2.35 g, 7.212 mmol), and [1,1 ′-bis(di-tert-butylphosphino)ferrocene]dichloropalladium(II) catalyst (0.0783 g, 0.120 mmol) in a sealed tube under an atmosphere of argon was added a freshly degassed mixture of dioxane:water (18:2, 15 mL) and the resulting mixture stirred at 90° C. overnight. The reaction was then cooled and diluted with EtOAc (40 mL) and washed with water (3×20 mL). The organic layer was concentrated under reduced pressure to afford crude tert-butyl 4 -(3 -cyano-4 -fluorophenyl)-2 -(2 -(pyridin-4 -ylmethoxy)pyridin-4 -yl)-1 H-pyrrolo[2,3 -b]pyridine-1 -carboxylate which was then taken up in EtOH 10 mL and hydrazine (5 mL, 1 M solution in EtOH) was then added and refluxed overnight to afford the crude tert-butyl 4 -(3 -amino-1 H-indazol-5 -yl)-2 -(2 -(pyridin-4 -ylmethoxy)pyridin-4 -yl)-1 H-pyrrolo[2,3 -b]pyridine-1 -carboxylate. The solvents were removed under reduced pressure and the crude product partitioned between EtOAC and water (100 mL, 50 %). The organic layer was collected, dried over anhydrous magnesium sulfate and concentrated under reduced pressure. The crude product was dissolved in THF (20 mL) and TBAF (5 mL, 1 M solution in THF) added and refluxed for 8 hours cooled concentrated and purified by HPLC to afford the title compound as a bright yellow solid (0.077 g, 10 %). ¹ H NMR (500 MHz, DMSO-d₆ ) δ 12.60 (s, 1 H), 8.83 (d, J=5.7 Hz, 2 H), 8.43 -8.38 (m, 2 H), 8.21 (d, J=5.5 Hz, 1 H), 7.92 -7.88 (m, 3 H), 7.69 -7.65 (m, 2 H), 7.54 (d, J=7.5 Hz, 2 H), 7.31 (d, J=5.0 Hz, 1 H), 5.69 (s, 2 H). LRMS (ESI) m/z for C₂₅H₁₉N₇₀ Molecular Weight: 433.4750 found 434.3 (M+H)⁺.

### Route to SU1634

### 4 -Bromo-2 -(pyridin-3 -ylmethoxy)pyridine

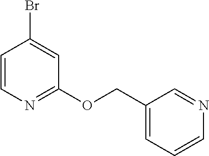 [SMILES: Brc1ccnc(OCc2cccnc2)c1]

To 4 -bromo-2 -fluoropyridine (0.2 g, 1.136 mmol) in anhydrous THF cooled to 0° C., potassium tert-butoxide (0.58 g g, 1.36 mmol in anhydrous THF (10 mL) was added under a blanket of argon. Pyridin-3 -ylmethanol (0.1488 g, 1.36 mmol) was then added and the resulting mixture allowed to warm up to room temperature and stirred for a further 4 hours. The reaction was then quenched by the pouring over ice/water (20 mL) and extracted with EtOAc (3×50 mL). The combined organic phase was washed with brine (3×20 mL), concentrated under reduced pressure and purified by flash column chromatography on silica gel eluting with a gradient from (0 to 50 %) EtOAc in petroleum ether to afford the target compound as a light brown oil in quantitative yield. ¹H NMR (500 MHz, DMSO-d₆ ) δ 8.68 (d, J=2.1 Hz, 1 H), 8.54 (dd, J=4.8, 1.7 Hz, 1 H), 8.10 (d, J=5.5 Hz, 1 H), 7.86 (dt, J=7.9, 2.0 Hz, 1 H), 7.41 (dd, J=7.8, 4.8 Hz, 1 H), 7.26 (dt, J=5.5, 1.5 Hz, 1 H), 7.21 (t, J=1.6 Hz, 1 H), 5.40 (s, 2 H). LRMS (ESI) m/z [M]⁺ C₁₁H₉BrN₂O Molecular Weight: 265.1100 found 265.0

### 2 -(Pyridin-3 -ylmethoxy)-4 -(4,4,5,5 -tetramethyl-1,3,2 -dioxaborolan-2 -yl)pyridine

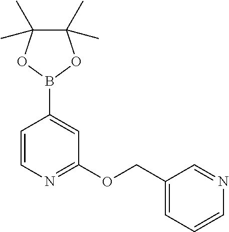 [SMILES: CC1(C)OB(c2ccnc(OCc3cccnc3)c2)OC1(C)C]

To a mixture of 4 -bromo-2 -(pyridin-3 -ylmethoxy)pyridine (1.72 g, 6.4878 mmol), [1,1 ′-bis(diphenylphosphino)ferrocene]dichloropalladium(II) catalyst (0.238 g, 0.325 mmol), potassium acetate (KOAc) (1.918 g, 19.541 mmol), and bis(pinacolato)diboron ((Bpin)₂ ) (2.15 g, 8.466 mmol) in a sealed tube under an atmosphere of argon was added a freshly degassed mixture of dioxane:water (18:2, 10 mL) and the resulting mixture stirred at 110° C. overnight. The reaction was then cooled and diluted with EtOAc (30 mL) and washed with water (3×20 mL). The organic layer was concentrated under reduced pressure and purified by flash column chromatography on silica gel eluting with a gradient (0 to 50 %) EtOAc in petroleum ether to afford the target compound as a brown solid (1.23 g, 61 %). ¹H NMR (500 MHz, DMSO-d₆ ) δ 8.71 (s, 1 H), 8.57 (s, 1 H), 8.22 (dd, J=5.0, 0.9 Hz, 1 H), 7.86 (dt, J=7.6, 1.9 Hz, 1 H), 7.41 (d, J=8.8 Hz, 1 H), 7.17 (dd, J=4.9, 0.9 Hz, 1 H), 7.04 (d, J=1.0 Hz, 1 H), 5.41 (s, 2 H), 1.30 (s, 12 H). LRMS (ESI) m/z for C₁₇H₂₁BN₂O₃ Molecular Weight: 312.1760 found 313.0 (M+H)⁺.

### tert-Butyl=4 -chloro-2 -(2 -(pyridin-3 -ylmethoxy)pyridin-4 -yl)-1 H-pyrrolo[2,3 -b]pyridine-i-carboxylate

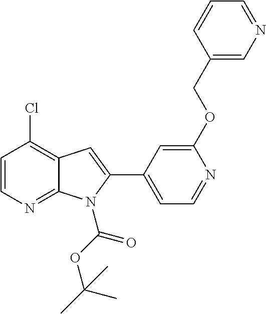 [SMILES: CC(C)(C)OC(=O)n1c(-c2ccnc(OCc3cccnc3)c2)cc2c(Cl)ccnc21]

To a mixture of tert-butyl 4 -chloro-2 -iodo-1 H-pyrrolo[2,3 -b]pyridine-1 -carboxylate (1.0 g, 2.64 mmol), 2 -(pyridin-3 -ylmethoxy)-4 -(4,4,5,5 -tetramethyl-1,3,2 -dioxaborolan-2 -yl)pyridine (1.23 g, 3.968 mmol), bis(triphenylphosphine)palladium(11 ) dichloride (0.0926 g, 0.132 mmol) and cesium carbonate (2.58 g, 7.9 mmol) in a sealed tube under an atmosphere of argon was added a freshly degassed mixture of dioxane:water (18:2, 10 mL) and the resulting mixture stirred at 80° C. overnight. The reaction was then cooled and diluted with EtOAc and washed with water (3×20 mL). The organic layer was concentrated under reduced pressure and purified by flash column chromatography on silica gel eluting with a gradient (0 to 50 %) EtOAc in petroleum ether+1 % triethylamine to afford the target compound as a brown solid (0.917 g, 80 %). ¹H NMR (500 MHz, DMSO-d₆ ) δ 8.68 (s, 1 H), 8.54 (s, 1 H), 8.22 (dd, J=4.9, 0.9 Hz, 1 H), 7.92 (s, 1 H), 7.87 (d, J=7.8 Hz, 1 H), 7.61 -7.35 (m, 3 H), 7.17 (dd, J=5.0, 0.9 Hz, 1 H), 7.04 (d, J=0.9 Hz, IH), 5.41 (s, 2 H), 1.30 (s, 12 H). LRMS (ESI) m/z [M]⁺ For C₂₃H₂₁ ClN₄O 3 Molecular Weight: 436.8960 found 438.0

### 5 -(2 -(2 -(Pyridin-3 -ylmethoxy)pyridin-4 -yl)-1 H-pyrrolo[2,3 -b]pyridin-4 -yl)-1 H-indazol-3 -amine SU1634

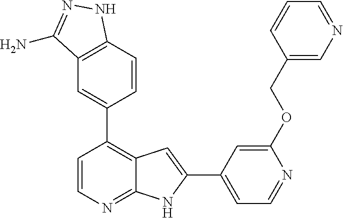 [SMILES: Nc1n[nH]c2ccc(-c3ccnc4[nH]c(-c5ccnc(OCc6cccnc6)c5)cc34)cc12]

To tert-butyl 4 -chloro-2 -(2 -(pyridin-3 -ylmethoxy)pyridin-4 -yl)-1 H-pyrrolo[2,3 -b]pyridine-1 -carboxylate (0.173 g, 0.513 mmol), (3 -cyano-4 -fluorophenyl)boronic acid (0.1396 g, 0.565 mmol), cesium carbonate (0.502 g, 1.541 mmol), and [1,1 ′-bis(di-tert-butylphosphino)ferrocene]dichloropalladium(II) catalyst (0.017 g, 0.0256 mmol) in a sealed tube under an atmosphere of argon was added a freshly degassed mixture of dioxane:water (18:2, 15 mL) and the resulting mixture stirred at 90° C. overnight. The reaction was then cooled and diluted with EtOAc (40 mL) and washed with water (3×20 mL). The organic layer was concentrated under reduced pressure to afford crude tert-butyl 4 -(3 -cyano-4 -fluorophenyl)-2 -(2 -(pyridin-3 -ylmethoxy)pyridin-4 -yl)-1 H-pyrrolo[2,3 -b]pyridine-1 -carboxylate following purification by flash column chromatography on silica gel eluting with a gradient of EtOAc in petroleum ether (20 to 100 %) as a brown solid (0.2679 54%). ¹H NMR (500 MHz, DMSO-d₆ ) δ 8.70 (d, J=2.2 Hz, 1 H), 8.54 (dd, J=4.9, 2.0 Hz, 2 H), 8.36 (dd, J=6.2, 2.4 Hz, 1 H), 8.29 (dd, J=5.3, 0.7 Hz, 1 H), 8.21 (ddd, J=8.7, 5.2, 2.4 Hz, 1 H), 7.89 (dt, J=7.9, 2.0 Hz, 1 H), 7.72 (t, J=9.0 Hz, 1 H), 7.53 (d, J=4.9 Hz, 1 H), 7.42 (ddd, J=7.8, 4.8, 0.9 Hz, 1 H), 7.23 (s, 1 H), 7.19 (dd, J=5.3, 1.5 Hz, 1 H), 7.09 (t, J=1.0 Hz, 1 H), 5.49 (s, 2 H), 1.36 (s, 9 H).

The crude product was then taken up in EtOH 10 mL and hydrazine (5 mL, 1 M solution in EtOH) was then added and refluxed overnight to afford the crude tert-butyl 4 -(3 -amino-1 H-indazol-5 -yl)-2 -(2 -(pyridin-3 -ylmethoxy)pyridin-4 -yl)-1 H-pyrrolo[2,3 -b]pyridine-1 -carboxylate. The solvents were removed under reduced pressure and the crude product partitioned between EtOAC and water (100 mL, 50 %). The organic layer was collected, dried over anhydrous magnesium sulfate and concentrated under reduced pressure. The crude product was dissolved in THF (20 mL), TBAF (5 mL, 1 M solution in THF) added and refluxed for 8 hours, cooled, concentrated and purified by HPLC to afford the title compound as a bright yellow solid (0.012 g, 5 %). ¹ H NMR (500 MHz, DMSO-d₆ ) δ 12.60 (s, 1 H), 8.92 (d, J=2.1 Hz, 1 H), 8.76 (dd, J=5.2, 1.5 Hz, 1 H), 8.50 -8.41 (m, 2 H), 8.35 -8.26 (m, 2 H), 7.94 (dd, J=8.7, 1.7 Hz, 1 H), 7.77 (dd, J=7.9, 5.1 Hz, 1 H), 7.72 (dd, J=5.5, 1.5 Hz, 1 H), 7.61 (d, J=1.4 Hz, 1 H), 7.59 -7.54 (m, 2 H), 7.35 (d, J=5.0 Hz, 1 H), 5.59 (s, 2 H). LRMS (ESI) m/z for C₂₅H₁ Nr₇0 Molecular Weight: 433.4750 found 434.3 (M+H)⁺.

### Route to SU1635

### 4 -Bromo-2 -(pyridin-2 -ylmethoxy)pyridine

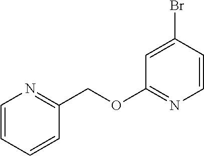 [SMILES: Brc1ccnc(OCc2ccccn2)c1]

To 4 -bromo-2 -fluoropyridine (0.2 g, 1.136 mmol) in anhydrous THF cooled to 0° C., potassium tert-butoxide (0.58 g g, 1.36 mmol in anhydrous THF (10 mL) was added under a blanket argon. Pyridin-2 -ylmethanol (0.1488 g, 1.36 mmol) was then added and the resulting mixture allowed to warm up to room temperature and stirred for a further 4 hours. The reaction was then quenched by the pouring over ice/water (20 mL) and extracted with EtOAc (3×50 mL). The combined organic phase was washed with brine (3×20 mL), concentrated under reduced pressure and purified by flash column chromatography on silica gel eluting with a gradient from (0 to 50 %) EtOAc in petroleum ether to afford the target compound as a light brown oil in quantitative yield. ¹H NMR (500 MHz, DMSO-d₆ ) δ 8.59 -8.53 (m, 2 H), 8.08 (d, J=5.5 Hz, 1 H), 7.43 -7.38 (m, 2 H), 7.30 (d, J=1.6 Hz, 1 H), 7.28 (dd, J=5.4, 1.7 Hz, 1 H), 5.44 (s, 2 H). LRMS (ESI) m/z [M]⁺ C₁₁H₉BrN₂O Molecular Weight: 265.1100 found 265.0, 266.0

### 2 -(Pyridin-2 -ylmethoxy)-4 -(4,4,5,5 -tetramethyl-1,3,2 -dioxaborolan-2 -yl)pyridine

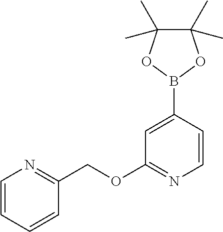 [SMILES: CC1(C)OB(c2ccnc(OCc3ccccn3)c2)OC1(C)C]

To a mixture of 4 -bromo-2 -(pyridin-2 -ylmethoxy)pyridine (2.45 g, 9.25 mmol), [1,1 ′-bis(diphenylphosphino)ferrocene]dichloropalladium(II) catalyst (0.0.335 g, 0.4625 mmol), potassium acetate (KOAc) (2.72 g, 27.7155 mmol) and bis(pinacolato)diboron ((Bpin)₂ ) (2.82 g, 11.1049 mmol) in a sealed tube under an atmosphere of argon was added a freshly degassed mixture of dioxane:water (18:2, 10 mL) and the resulting mixture stirred at 110° C. overnight. The reaction was then cooled and diluted with EtOAc (30 mL) and washed with water (3×20 mL). The organic layer was concentrated under reduced pressure and purified by flash column chromatography on silica gel eluting with a gradient (0 to 50 %) EtOAc in petroleum ether to afford the target compound as a brown solid (1.21 g, 42 %). ¹H NMR (500 MHz, DMSO-d₆ ) δ 8.55 (dt, J=4.8, 1.2 Hz, 1 H), 8.19 (dd, J=4.9, 0.9 Hz, 1 H), 7.92 (s, 1 H), 7.79 (td, J=7.7, 1.8 Hz, 1 H), 7.44 (d, J=7.8 Hz, 1 H), 7.34 -7.28 (m, 1 H), 7.17 (dd, J=4.9, 0.9 Hz, 1 H), 7.10 (s, 1 H), 5.44 (s, 2 H), 1.32 (s, 12 H). LRMS (ESI) m/z [M]⁺ For C₁₇H₂₁BN₂O₃ Molecular Weight: 312.1760 found 313.0

### tert-Butyl-4 -chloro-2 -(2 -(pyridin-2 -ylmethoxy)pyridin-4 -yl)-1 H-pyrrolo[2,3 -b]pyridine-1 -carboxylate

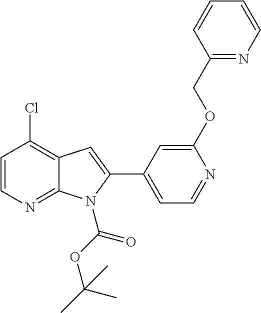 [SMILES: CC(C)(C)OC(=O)n1c(-c2ccnc(OCc3ccccn3)c2)cc2c(Cl)ccnc21]

To a mixture of tert-butyl 4 -chloro-2 -iodo-1 H-pyrrolo[2,3 -b]pyridine-1 -carboxylate (1.0 g, 2.64 mmol), 2 -(pyridin-2 -ylmethoxy)-4 -(4,4,5,5 -tetramethyl-1,3,2 -dioxaborolan-2 -yl)pyridine (1.21 g, 3.885 mmol) bis(triphenylphosphine)palladium(II) dichloride (0.0926 g, 0.132 mmol) and cesium carbonate (2.58 g, 7.9 mmol) in a sealed tube under an atmosphere of argon was added a freshly degassed mixture of dioxane:water (18:2, 10 mL) and the resulting mixture stirred at 80° C. overnight. The reaction was then cooled and diluted with EtOAc and washed with water (3×20 mL). The organic layer was concentrated under reduced pressure and purified by flash column chromatography on silica gel eluting with a gradient (0 to 50 %) EtOAc in petroleum ether+1 % triethylamine to afford the target compound as a crude product (0.32 g, 28 %) that was progressed directly to the next stage.

### 5 -(2 -(2 -(Pyridin-2 -ylmethoxy)pyridin-4 -yl)-1 H-pyrrolo[2,3 -b]pyridin-4 -yl)-1 H-indazol-3 -amine SU1635

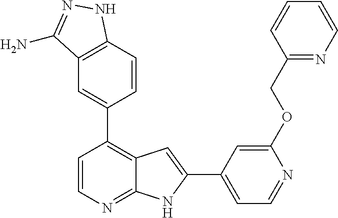 [SMILES: Nc1n[nH]c2ccc(-c3ccnc4[nH]c(-c5ccnc(OCc6ccccn6)c5)cc34)cc12]

To tert-butyl 4 -chloro-2 -(2 -(pyridin-2 -ylmethoxy)pyridin-4 -yl)-1 H-pyrrolo[2,3 -b]pyridine-1 -carboxylate (0.32 g, 0.95 mmol), (3 -cyano-4 -fluorophenyl)boronic acid (0.172 g, 1.045 mmol) Cesium carbonate (0.928 g, 2.85 mmol), and [1,1 ′-bis(di-tert-butylphosphino)ferrocene]dichloropalladium(II) catalyst (0.031 g, 0.0475 mmol) in a sealed tube under an atmosphere of argon was added a freshly degassed mixture of dioxane:water (18:2, 15 mL) and the resulting mixture stirred at 90° C. overnight. The reaction was then cooled and diluted with EtOAc (40 mL) and washed with water (3×20 mL). The organic layer was concentrated under reduced pressure to afford crude tert-butyl 4 -(3 -cyano-4 -fluorophenyl)-2 -(2 -(pyridin-2 -ylmethoxy)pyridin-4 -yl)-1 H-pyrrolo[2,3 -b]pyridine-1 -carboxylate following purification by flash column chromatography on silica gel eluting with a gradient of EtOAc in petroleum ether (20 to 100 %) as a brown solid. (0.253 g, 51 %). The crude product was then taken up in EtOH 10 mL and hydrazine (5 mL, 1 M solution in EtOH) was then added and refluxed overnight to afford the crude tert-butyl 4 -(3 -amino-1 H-indazol-5 -yl)-2 -(2 -(pyridin-2 -ylmethoxy)pyridin-4 -yl)-1 H-pyrrolo[2,3 -b]pyridine-1 -carboxylate. The solvents were removed under reduced pressure and the crude product partitioned between EtOAC and water (100 mL, 50 %). The organic layer was collected, dried over anhydrous magnesium sulfate and concentrated under reduced pressure. The crude product was dissolved in THF (20 mL) and TBAF (5 mL, 1 M solution in THF) added and refluxed for 8 hours cooled concentrated and purified by HPLC to afford the title compound as a bright yellow solid (0.082 g, 20 %). ⁴ H NMR (500 MHz, DMSO-d₆ ) δ 12.56 (s, 1 H), 8.87 (dd, J=5.4, 2.1 Hz, 1 H), 8.72 (td, J=5.0, 1.5 Hz, 1 H), 8.40 -8.37 (m, 1 H), 8.26 (d, J=5.5 Hz, 1 H), 7.89 (dd, J=8.7, 1.7 Hz, 1 H), 7.73 (dt, J=7.8, 4.1 Hz, 1 H), 7.66 (dd, J=5.5, 1.5 Hz, 1 H), 7.56 (d, J=1.4 Hz, 1 H), 7.54 -7.50 (m, 1 H), 7.30 (d, J=4.9 Hz, 1 H), 5.54 (s, 2 H). LRMS (ESI) m/z for C₂₅H₁₉N₇₀ Molecular Weight: 433.4750 found 434.3 (M+H)⁺.

### Benzyl Isosteres of the Benzyloxy Analogues of the 2 -Aryl Azaindole Series

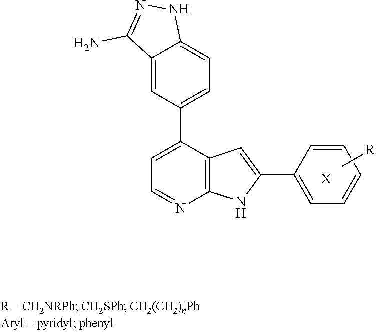

### Route to SU1352

### N-Benzyl-5 -(4 -chloro-1 H-pyrrolo[2,3 -b]pyridin-2 -yl)pyridin-2 -ylamine

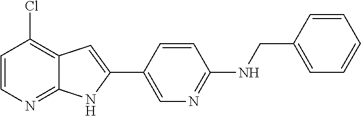 [SMILES: Clc1ccnc2[nH]c(-c3ccc(NCc4ccccc4)nc3)cc12]

A suspension of 4 -chloro-2 -iodo-7 -azaindole (0.174 g, 0.62 mmol), N-benzyl-5 -(4,4,5,5 -tetramethyl-1,3,2 -dioxaborolan-2 -yl)pyridin-2 -amine (0.210 g, 0.68 mmol), K₂ CO₃ (0.305 g, 2.21 mmol) and Bis(triphenylphosphine) palladium(II) chloride (0.048 g, 0.068 mmol) in 1,4 -dioxane (3 mL) and water (2 mL) was degassed under nitrogen. The reaction mixture was heated to 100° C. for 22 h. The reaction mixture was cooled to room temperature and extracted into EtOAc, washed with water, aqueous saturated NaHCO₃ and brine. Organics were concentrated under reduced pressure and the resulting solid triturated with hexane and Et₂O to afford the title compound. This material was used crude in the next reaction (0.023 g). ¹ H NMR (DMSO-d₆ ): δ 4.55 (d, J=5.6 Hz, 1 H), 6.61 (d, J=8.8 Hz, 1 H), 6.74 (s, 1 H), 7.12 (d, J=5.2 Hz, 1 H), 7.23 (m, 1 H), 7.34 (m, 3 H), 7.41 (t, J=6.0 Hz, 1 H), 7.96 (dd, J=2.4, 8.8 Hz, 1 H), 8.07 (d, J=5.2 Hz, 1 H), 8.61 (d, J=2.0 Hz, 1 H), 12.30 (br s, 1 H).

### 5 -(2 -(6 -(Benzylamino)pyridin-3 -yl)-1 H-pyrrolo[2,3 -b]pyridin-4 -yl)-1 H-indazol-3 -amine SU1352

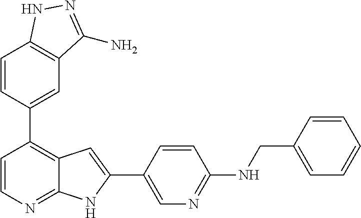 [SMILES: Nc1n[nH]c2ccc(-c3ccnc4[nH]c(-c5ccc(NCc6ccccc6)nc5)cc34)cc12]

To a suspension of N-Benzyl-5 -(4 -chloro-1 H-pyrrolo[2,3 -b]pyridin-2 -yl)pyridin-2 -ylamine (0.020 g, 0.06 mmol), 5 -(4,4,5,5 -tetramethyl-1,3,2 -dioxaborolan-2 -yl)-1 H-indazol-3 -amine (0.026 g, 0.10 mmol) and [1,1 ′-bis(di-tert-butylphosphino)ferrocene]dichloropalladium(II) catalyst (0.009 g, 0.014 mmol) in EtOH (3.0 mL, degassed under nitrogen) was added K₃ PO₄ (1 M, 1.2 mL) and the reaction mixture was heated to 120° C. for 20 h. The reaction mixture was cooled to room temperature, dissolved in DMF (1 mL) and filtered through a pad of cotton wool. Purification was achieved using HPLC (see conditions) to afford the title compounds as yellow solid (0.010 g, 0.023 mmol, 38 %). ¹H NMR (DMSO-d₆ ): δ 4.61 (s, 2 H), 6.94 (br s, 1 H), 7.14 (s, 1 H), 7.23 (d, J=4.0 Hz, 1 H), 7.30 (m, 1 H), 7.37 (m, 4 H), 7.45 (d, J=8.5 Hz, 1 H), 7.79 (d, J=9.0 Hz, 1 H), 8.26 (d, J=5.0 Hz, 1 H), 8.28 (s, 1 H), 8.54 (s, 1 H), 12.20 (br s, 1 H). HRMS: For C₂₆H₂₂N₇ requires 432.1931 found 432.1929 . (M+H)⁺.

### 5 -(2 -(2 -(Benzylamino)pyridin-4 -yl)-1 H-pyrrolo[2,3 -b]pyridin-4 -yl)-1 H-indazol-3 -amine SU1357

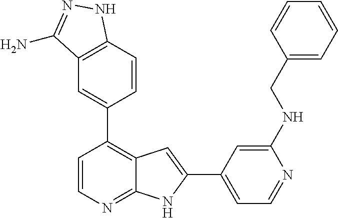 [SMILES: Nc1n[nH]c2ccc(-c3ccnc4[nH]c(-c5ccnc(NCc6ccccc6)c5)cc34)cc12]

A 0-5-2 mL Biotage MW tube was charged with 5-(2-(2 -fluoropyridin-4 -yl)-1 H-pyrrolo[2,3 -b]pyridin-4 -yl)-1 H-indazol-3 -amine (SU1339 ) (17.2 mg, 0.05 mmol, 1 eq.), phenylmethanamine (21.8 μL, 0.2 mmol, 4 eq.) and anhydrous DMSO (600 μL). The reaction mixture was then flushed with N₂ and heated to 120° C. overnight. Purification by reverse phase HPLC gave the title compound as a yellow solid (16 mg). ¹H NMR (400 MHz, DMSO-D₆ ) δ 4.67 (d, J=4.6 Hz, 2 H), 7.29 (d, J=5.0 Hz, 1 H), 7.33 (d, J=7.0 Hz, 1 H), 7.37-7.47 (m, 5 H), 7.49 (d, J=6.4 Hz, 1 H), 7.57 (d, J=13.5 Hz, 2 H), 7.75 (d, J=8.8 Hz, 1 H), 8.04 (d, J=6.6 Hz, 1 H), 8.28 (s, 1 H), 8.42 (d, J=5.0 Hz, 1 H), 12.72 (s, 1 H). NH₂ and 2 NH peaks not observed or very broad.

### 5-(2-(2-(Benzyl(methyl)amino)pyridin-4 -yl)-1 H-pyrrolo[2,3 -b]pyridin-4 -yl)-1 H-indazol-3 -amine SU1614

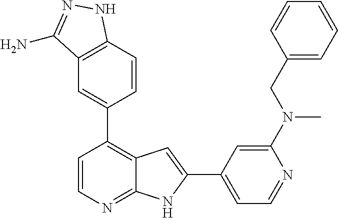 [SMILES: CN(Cc1ccccc1)c1cc(-c2cc3c(-c4ccc5[nH]nc(N)c5c4)ccnc3[nH]2)ccn1]

A 0-5-2 mL Biotage MW tube was charged with 5-(2-(2 -fluoropyridin-4 -yl)-1 H-pyrrolo[2,3 -b]pyridin-4 -yl)-1 H-indazol-3 -amine (SU1339 ) (17.2 mg, 0.05 mmol, 1 eq.), N-methyl-1 -phenylmethanamine (24.2 mg, 25.8 μL, 0.2 mmol, 4 eq.) and anhydrous DMSO (750 μL). The reaction mixture was then flushed with N₂ and heated to 110° C. for 3 days. Purification by reverse phase HPLC gave the title compound as a yellow solid (13.4 mg). ¹H NMR (400 MHz, DMSO) δ 3.23 (s, 3 H), 4.97 (s, 2 H), 7.26-7.35 (m, 4 H), 7.35-7.43 (m, 2 H), 7.44-7.51 (m, 2 H), 7.64 (d, J=15.7 Hz, 2 H), 7.76-7.84 (m, 1 H), 8.07 (d, J=6.4 Hz, 1 H), 8.31 (s, 1 H), 8.41 (d, J=5.0 Hz, 1 H), 11.76-12.04 (bs, 1 H), 12.66 (s, 1 H). NH2 peak not observed. LRMS: Calculated for C₂₇H₂₃N₇ 445.20 ; Found: 446.3 [M+H]⁺

### 5-(2-(2-(Benzylthio)pyridin-4 -yl)-1 H-pyrrolo[2,3 -b]pyridin-4 -yl)-1 H-indazol-3 -amine SU1615

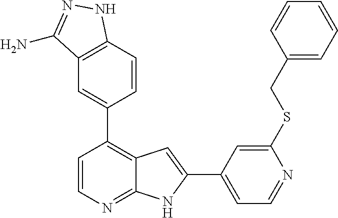 [SMILES: Nc1n[nH]c2ccc(-c3ccnc4[nH]c(-c5ccnc(SCc6ccccc6)c5)cc34)cc12]

A 0-5-2 mL Biotage MW tube was charged with 5-(2-(2 -fluoropyridin-4 -yl)-1 H-pyrrolo[2,3 -b]pyridin-4 -yl)-1 H-indazol-3 -amine (SU1339 ) (17.2 mg, 0.05 mmol, 1 eq.), phenylmethanethiol (44.7 mg, 42.2 μL, 036 mmol, 6 eq.) and anhydrous NMP (0.8 mL) followed by sodium methoxide (16.2 mg, 0.3 mmol, 5 eq.) The reaction mixture was then flushed with N₂ and heated to 60° C. overnight. Purification by reverse phase HPLC gave the title compound as a yellow solid. ¹H NMR (500 MHz, DMSO) δ 4.50 (s, 2 H), 7.21-7.26 (m, 1 H), 7.27 (d, J=5.0 Hz, 1 H), 7.29-7.33 (m, 2 H), 7.43-7.46 (m, 2 H), 7.48 (d, J=8.7 Hz, 1 H), 7.50 (d, J=2.2 Hz, 1 H), 7.72 (dd, J=5.3, 1.6 Hz, 1 H), 7.80-7.85 (m, 1 H), 7.92 (s, 1 H), 8.32 (d, J=1.5 Hz, 1 H), 8.36(d, J=4.9 Hz, 1 H), 8.51 (d, J=5.3 Hz, 1 H), 12.51 (s, 1 H). NH₂ and (1 ) NH peaks not observed.

LRMS: Calculated for C₂₆H₂₀N₆ S 448.15 Found: 449.2 [M+H]⁺

### 5-(2-(2-(Benzylthio)-6 -morpholinopyridin-4 -yl)-1 H-pyrrolo[2,3 -b]pyridin-4 -yl)-1 H-indazol-3 -amine SU1631

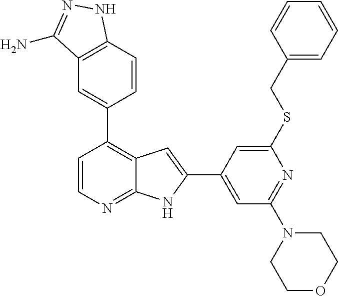 [SMILES: Nc1n[nH]c2ccc(-c3ccnc4[nH]c(-c5cc(SCc6ccccc6)nc(N6CCOCC6)c5)cc34)cc12]

A 0.5-2 mL Biotage tube was charged with 5-(2-(2,6 -difluoropyridin-4 -yl)-1 H-pyrrolo[2,3 -b]pyridin-4 -yl)-1 H-indazol-3 -amine (SU1361 ) (29 mg, 0.08 mmol, 1 eq.), phenylmethanethiol (10.9 mg, 10.3 μL, 0.088 mmol, 1.1 eq.) sodium methoxide (4.75 mg, 0.088 mmol, 1.1 eq.) and anhydrous 1,3 -dimethyl-2 -imidazolidinone (1 mL) and sealed under an argon atmosphere. The reaction mixture was heated to 90° C. for 19 hours and then cooled to room temperature, diluted with morpholine (250 μL) and heated to 210° C. under argon for 20 minutes under microwave irradiation. The reaction mixture was purified by HPLC to give the title compound as a yellow solid (18.3 mg, 30 %). ¹H NMR (500 MHz, DMSO) δ 3.56-3.61 (m, 4 H), 3.70-3.76 (m, 4 H), 4.43 (s, 2 H), 7.14 (s, 1 H), 7.20-7.24 (m, 2 H), 7.25 (d, J=5.0 Hz, 1 H), 7.27-7.33 (m, 2 H), 7.39-7.44 (m, 3 H), 7.50 (d, J=8.7 Hz, 1 H), 7.84 (dd, J=8.7, 1.7 Hz, 1 H), 8.32 (s, 1 H), 8.34 (d, J=5.0 Hz, 1 H), 12.38 (s, 1 H). NH₂ and (1 ) NH peaks not observed.

### 5-(2-(3 -Phenethylphenyl)-1 H-pyrrolo[2,3 -b]pyridin-4 -yl)-1 H-indazol-3 -amine SU1607

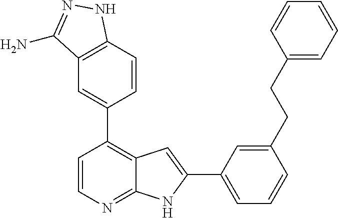 [SMILES: Nc1n[nH]c2ccc(-c3ccnc4[nH]c(-c5cccc(CCc6ccccc6)c5)cc34)cc12]

A 2-5 mL Biotage MW tube was charged with methyl 4 -chloro-2-(3 -phenethylphenyl)-1 H-pyrrolo[2,3 -b]pyridine (100 mg, 0.3 mmol), 5-(4,4,5,5 -tetramethyl-1,3,2 -dioxaborolan-2 -yl)-1 H-indazol-3 -amine (103 mg, 0.4 mmol, 1.3 eq.), [1,1 ′-bis(di-tert-butylphosphino)ferrocene]dichloropalladium(II) catalyst (9.8 mg, 0.015 mmol, 5 mol %) and dioxane (1.8 mL). The resulting suspension was purged with nitrogen for 5 minutes before adding 1 M aq. K₃ PO₄ (1.8 mL). The reaction mixture was heated to 110° C. for 16 hrs. The reaction mixture was then allowed to cool to room temperature, diluted with water (10 mL) and extracted with EtOAc (3×20 mL). The combined organic fractions were then dried over anhydrous magnesium sulfate, filtered and the solvent removed under reduced pressure. The crude product was then purified by HPLC to give the desired product as a pale yellow solid (32 mg). ¹H NMR (500 MHz, DMSO-D₆ ) δ 2.97 (m, 4 H), 7.10-7.30 (m, 8 H), 7.38 (t, J=7.6 Hz, 1 H), 7.51 (d, J=8.7 Hz, 1 H), 7.82 (t, J=10.2 Hz, 1 H), 7.90 (s, 1 H), 8.28 (m, 2 H), 12.32 (s, 1 H).

LRMS: Calculated for C₂₈H₂₃N₅ 429.20 ; Found:430.20 (M+H)⁺.

### Route to SU1621

### 2 -phenethoxy-4-(4,4,5,5 -tetramethyl-1,3,2 -dioxaborolan-2 -yl)pyridine

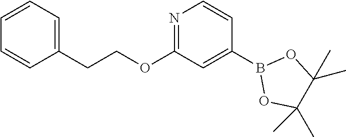 [SMILES: CC1(C)OB(c2ccnc(OCCc3ccccc3)c2)OC1(C)C]

To 4 -bromo-2 -fluoropyridine (0.415 g, 2.372 mmol) in anhydrous THF cooled to 0° C., potassium tert-butoxide (0.291 g, 2.5945 ) mmol in anhydrous THF (10 mL) was added under a blanket of argon. Pyridin-2 -ylmethanol (0.316 g, 2.5945 mmol) was then added and the resulting mixture allowed to warm up to room temperature and stirred for a further 4 hours. The reaction was then quenched by the pouring over ice/water (20 mL) and extracted with EtOAc (3×50 mL). The combined organic phase was washed with brine (3×20 mL), concentrated under reduced pressure and purified by flash column chromatography on silica gel eluting with a gradient from (0 to 50 %) EtOAc in petroleum ether to afford 4 -bromo-2 -phenethoxypyridine as a light brown oil. To the obtained 4 -bromo-2-(pyridin-2 -ylmethoxy)pyridine (0.52 g, 1.87 mmol) was added [1,1 ′-bis(diphenylphosphino)ferrocene]dichloropalladium(II) catalyst (0.1368 g, 0.1869 mmol), potassium acetate (KOAc) (0.55 g, 5.61 mmol), and bis(pinacolato)diboron ((Bpin)₂ ) (0.57 g, 11.1049 mmol) in a sealed tube under an atmosphere of argon followed by a freshly degassed mixture of dioxane:water (18:2, 10 mL) and the resulting mixture stirred at 110° C. overnight. The reaction was then cooled and diluted with EtOAc (30 mL) and washed with water (3×20 mL). The organic layer was concentrated under reduced pressure and purified by flash column chromatography on silica gel eluting with a gradient (0 to 50 %) EtOAc in petroleum ether to afford the target compound as a brown solid (0.307 g, 40 %). ¹H NMR (500 MHz, DMSO-d₆ ) δ 8.19 (dd, J=4.9, 0.9 Hz, 1 H), 7.31 (d, J=4.9 Hz, 4 H), 7.22 (d, J=4.3 Hz, 1 H), 7.12 (dd, J=4.9, 0.9 Hz, 1 H), 6.91 (d, J=1.0 Hz, 1 H), 4.46 (t, J=6.8 Hz, 2 H), 3.02 (t, J=6.8 Hz, 2 H), 1.30 (s, 12 H). LRMS (ESI) m/z [M]⁺ For C₁₉ Hz₄BNO₃ Molecular Weight: 325.2150 found 326.1

### tert-Butyl 4 -chloro-2-(2 -phenethoxypyridin-4 -yl)-1 H-pyrrolo[2,3 -b]pyridine-1 -carboxylate

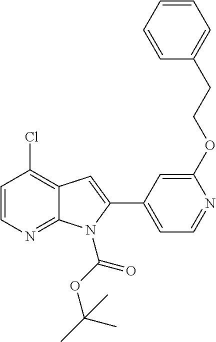 [SMILES: CC(C)(C)OC(=O)n1c(-c2ccnc(OCCc3ccccc3)c2)cc2c(Cl)ccnc21]

To a mixture of tert-butyl 4 -chloro-2 -iodo-1 H-pyrrolo[2,3 -b]pyridine-1 -carboxylate (0.297 g, 0.7857 mmol), 2 -phenethoxy-4-(4,4,5,5 -tetramethyl-1,3,2 -dioxaborolan-2 -yl)pyridine (0.307 g, 9.44 mmol), bis(triphenylphosphine)palladium(II) dichloride (0.0287 g, 0.0392 mmol) and cesium carbonate (0.768 g, 2.357 mmol) in a sealed tube under an atmosphere of argon was added a freshly degassed mixture of dioxane:water (18:2, 10 mL) and the resulting mixture stirred at 80° C. overnight. The reaction was then cooled and diluted with EtOAc and washed with water (3×20 mL). The organic layer was concentrated under reduced pressure and purified by flash column chromatography on silica gel eluting with a gradient (0 to 50 %) EtOAc in petroleum ether+1 % triethylamine to afford the target compound as A crude product (0.23 g, 66 %) that was progressed directly to the next stage.

### 5-(2-(2 -Phenethoxypyridin-4 -yl)-1 H-pyrrolo[2,3 -b]pyridin-4 -yl)-1 H-indazol-3 -amine SU1621

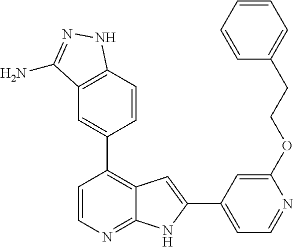 [SMILES: Nc1n[nH]c2ccc(-c3ccnc4[nH]c(-c5ccnc(OCCc6ccccc6)c5)cc34)cc12]

To tert-butyl 4 -chloro-2-(2 -phenethoxypyridin-4 -yl)-1 H-pyrrolo[2,3 -b]pyridine-1 -carboxylate (0.2329 g, 0.5176 mmol), 5-(4,4,5,5 -tetramethyl-1,3,2 -dioxaborolan-2 -yl)-1 H-indazol-3 -amine (0.1609 g, 0.6211 mmol), cesium carbonate (0.768 g, 2.357 mmol), and [1 ,1′-bis(di-tert-butylphosphino)ferrocene]dichloropalladium(II) catalyst (0.0256 g, 0.0392 mmol) in a sealed tube under an atmosphere of argon was added a freshly degassed mixture of dioxane:water (18:2, 15 mL) and the resulting mixture stirred at 110° C. overnight. The reaction was then cooled and diluted with EtOAc (40 mL) and washed with water (3×20 mL). The organic layer was concentrated under reduced pressure. The crude product was dissolved in THF (20 mL) and TBAF (5 mL, 1 M solution in THF) added and refluxed for 8 hours cooled concentrated and purified by HPLC to afford the title compound as a bright yellow solid (0.0265 g, 11 %). ¹ H NMR (400 MHz, DMSO-d₆ ) δ 12.56-12.13 (m, 1 H), 11.58 (s, 1 H), 8.34 (d, J=5.0 Hz, 1 H), 8.27 -8.23 (m, 1 H), 8.22 (d, J=5.5 Hz, 1 H), 7.74 (dd, J=8.7, 1.6 Hz, 1 H), 7.59 (dd, J=5.4, 1.5 Hz, 1 H), 7.47 (d, J=1.9 Hz, 1 H), 7.44-7.39 (m, 2 H), 7.37-7.30 (m, 4 H), 7.25 (dd, J=5.6, 2.1 Hz, 2 H), 5.56 (s, 2 H), 4.54 (t, J=6.9 Hz, 2 H), 3.08 (t, J=6.8 Hz, 2 H). LRMS (ESI) m/z for Chemical Formula: C₂₇H₂₂N₆O Molecular Weight: 446.5140 found 446.3 (M+H)⁺.

(Hetero)Aromatic Moiety with Different Methylamino Substituents at Position 2 of the Azaindole Ring

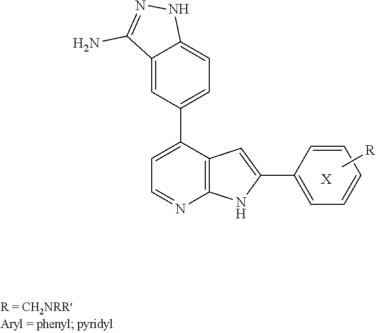

### 3-(4 -Chloro-7 -azaindol-2 -yl)benzaldehyde

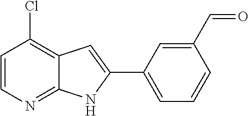 [SMILES: O=Cc1cccc(-c2cc3c(Cl)ccnc3[nH]2)c1]

A suspension of 4 -chloro-2 -iodo-7 -azaindole (0.3 g, 1.08 mmol), (3 -formylphenyl) boronic acid (0.2 g, 1.29 mmol), 2 M potassium carbonate solution (1.08 ml, 2.15 mmol) in 1,4 dioxane (3 ml) was deoxygenated with nitrogen in sealed tube. Then [1,1 ′-bis(diphenylphosphino)ferrocene]dichloropalladium(II) catalyst (0.044 g, 0.054 mmol) was added and the tube was sealed and the mixture allowed to stirred at 80° C. for 18 h. The reaction mixture then poured into water and filtered. The solid participate was triturated with petroleum ether and EtOAc to afford the desired compound as dark brown solid (0.24 g, 86 %), ¹ H NMR (400 MHz, DMSO-d₆ ) δ ppm 7.17 (s, 1 H) 7.24 (d, J=5.27 Hz, 1 H) 7.70-7.77 (m, 1 H) 7.91 (d, J=7.47 Hz, 1 H) 8.21 (d, J=5.27 Hz, 1 H) 8.33 (d, J=7.47 Hz, 1 H) 8.57 (s, 1 H) 10.09 (s., 1 H). ¹³ C NMR (100 MHz, DMSO-dc) 6 ppm 96.80, 116.64, 120.37, 127.54, 129.44, 130.45, 131.78, 132.47, 134.50, 137.50, 137.50, 138.58, 144.54, 150.82, 193.45 . m/z (ESI-MS) [M]⁺ 257.1.

### 4-(4 -Chloro-7 -azaindol-2 -yl)-2, 6 -difluorobenzaldehyde

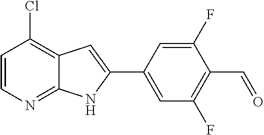 [SMILES: O=Cc1c(F)cc(-c2cc3c(Cl)ccnc3[nH]2)cc1F]

A suspension of 4 -chloro-2 -iodo-7 -azaindole (0.3 g, 1.08 mmol), 3,5 -difluoro-4 -formylphenyl)boronic acid (0.24 g, 1.29 mmol), 2 M potassium carbonate solution (1.08 ml, 2.15 mmol) in 1,4 dioxane (3 ml) was deoxygenated with nitrogen in sealed tube. Then [1,1 ′-bis(diphenylphosphino)ferrocene]dichloropalladium(II) catalyst (0.044 g, 0.054 mmol) was added and the tube was sealed and the mixture allowed to stirred at 80° C. for 18 h. The reaction mixture then poured into water and filtered. The solid participate was triturated with petroleum ether and EtOAc to afford the desired compound as dark brown solid (0.3 g, 95 %), ¹ H NMR (400 MHz, DMSO-d₆ ) δ ppm 7.29 (d, J=4.83 Hz, 1 H) 7.48 (s, 1 H) 7.96 (s, 1 H) 7.99 (s, 1 H) 8.29 (d, J=5.27 Hz, 1 H) 10.21 (s., 1 H). ¹³ C NMR (100 MHz, DMSO-d₆ ) δ ppm 97.68, 100.33, 109.51, 109.78, 117.02, 119.98, 132.83, 135.46, 139.41, 146.04, 150.80, 164.55, 184.77. ¹⁹ F NMR (DMSO-d₆ ) -114.79 . m/z (ESI-MS) [M]⁺ 587.1.

### General Procedure A:

The appropriate aldehyde (1 eq.) and required amine (1.3 eq.) were allowed to react in the presence of sodium triacetoxyborohydride (1.5 eq.) and acetic acid (1.3 eq.) in dimethylacetamide (2 ml) at room temperature for 48 h. The reaction mixture was then poured into 1 M sodium carbonate solution and stirred in an ice bath for 3 h, then filtered. The solid was then collected and purified by column chromatography.

### General Procedure B:

A suspension of the required chloroaryl substrate (1 eq.), 5-(4,4,5,5 -tetramethyl-1,3,2 -dioxaborolan-2 -yl)-1 H-indazol-3 -amine (1.2-1.5 eq.), base (2 eq.) in 1:3 of solvent was deoxygenated with nitrogen in a sealed tube. Then [1,1 ′-bis(di-tert-butylphosphino)ferrocene]dichloropalladium(li) catalyst (0.05 eq.) was added, the tube sealed and the mixture stirred at 90-100° C. for 18 h. After the reaction was cooled to room temperature, EtOAc and water were added. The extracted organic layer was dried over magnesium sulfate and concentrated under reduced pressure and the residue purified by column chromatography.

### Route to SU1549

### N-[3-(4 -Chloro-7 -azaindole)benzyl]-N-phenylamine

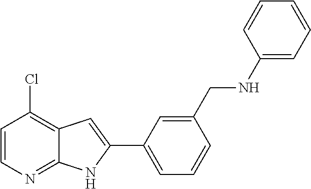 [SMILES: Clc1ccnc2[nH]c(-c3cccc(CNc4ccccc4)c3)cc12]

3-(4 -Chloro-7 -azaindol-2 -yl)benzaldehyde (0.12 g, 0.46 mmol) was reacted with aniline (0.057 ml, 0.6 mmol), sodium triacetoxyborohydride (0.15 g, 0.7 mmol) and acetic acid (0.037 ml, 0.6 mmol) as described in general procedure A and chromatographic purification (10-30 % EtOAc in petroleum ether 60-80 %) afforded the desired compound as a white solid (0.12 g, 77 %), 1 H NMR (400 MHz, DMSO-d6 ) δ ppm 4.32 (d, J=6.15 Hz, 2 H) 6.27 (t, J=5.93 Hz, 1 H) 6.51 (t, J=7.25 Hz, 1 H) 6.62 (d, J=7.47 Hz, 2 H) 6.97 (s, 1 H) 7.02-7.08 (m, 2 H) 7.21 (d, J=5.27 Hz, 1 H) 7.38 (d, J=7.91 Hz, 1 H) 7.43 (t, J=7.69 Hz, 1 H) 7.85 (d, J=7.47 Hz, 1 H) 8.04 (s, 1 H) 8.17 (d, J=5.27 Hz, 1 H) 12.54 (br. s., 1 H). 13 C NMR (100 MHz, DMSO-d6 ) δ ppm 47.05, 95.56, 112.94, 116.43, 120.44, 124.48, 125.38, 128.02, 129.41, 129.53, 131.42, 134.03, 140.04, 141.78, 143.96, 144.36, 149.17, 150.07. 6 ppm. m/z (ESI-MS) [M]+334.1.

### 5-(2-(3-((Phenylamino)methyl)phenyl)-1 H-pyrrolo[2,3 -b]pyridin-4 -yl)-1 H-indazol-3 -amine SU1549

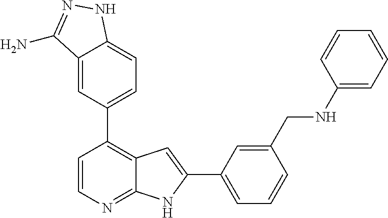 [SMILES: Nc1n[nH]c2ccc(-c3ccnc4[nH]c(-c5cccc(CNc6ccccc6)c5)cc34)cc12]

N-[3-(4 -Chloro-7 -azaindole)benzyl]-N-phenylamine (0.07 g, 0.2 mmol), 5-(4,4,5,5 -tetramethyl-1,3,2 -dioxaborolan-2 -yl)-1 H-indazol-3 -amine (0.06 g, 0.25 mmol), 2 M potassium carbonate solution (0.2 ml, 0.4 mmol), [1,1 ′-bis(di-tert-butylphosphino)ferrocene]dichloropalladium(II) catalyst (0.006 g, 0.01 mmol) in 0.6 ml 1,4 dioxane were reacted as described in general procedure B and chromatographic purification (30-100 % EtOAc in petroleum ether 60-80 %), further purification by HPLC gave the titled compound as a yellow solid (14.4 mg, 16 %), ¹ H NMR (400 MHz, DMSO-d₆ ) δ ppm 4.31 (d, J=5.71 Hz, 2 H) 5.56 (s, 2 H) 6.20-6.26 (m, 1 H) 6.48-6.54 (m, 1 H) 6.61 (d, J=7.91 Hz, 3 H) 7.04 (t, J=7.69 Hz, 3 H) 7.16 (d, J=1.32 Hz, 1 H) 7.19 (d, J=4.83 Hz, 1 H) 7.32-7.36 (m, 1 H) 7.41 (d, J=7.91 Hz, 2 H) 7.71 (d, J=9.67 Hz, 1 H) 7.85 (d, J=7.47 Hz, 1 H) 8.26 (d, J=5.27 Hz, 1 H) 12.22 (br. s., 1 H). m/z (ESI-HRMS) calculated for C₂₇H₂₃N_{6'}₂ 431.179 found=431.1978 . (M+H)⁺.

### Route to SU1547

### N-[3-(4 -Chloro-7 -azaindole)benzyl]-N-(tert-butyl)amine

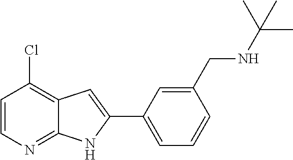 [SMILES: CC(C)(C)NCc1cccc(-c2cc3c(Cl)ccnc3[nH]2)c1]

3-(4 -Chloro-7 -azaindol-2 -yl)benzaldehyde (0.2 g, 0.78 mmol) was reacted with tert-butylamine (0.1 ml, 1.0 mmol), sodium triacetoxyborohydride (0.24 g, 1.17 mmol) and acetic acid (0.057 ml, 1 mmol) as described in general procedure A and chromatographic purification (0-10 % methanol in EtOAc) afforded the desired compound as a white solid (0.15 g, 62 %), ¹ H NMR (400 MHz, CDCl₃ ) δ ppm 1.67 (s, 9 H) 4.09 (br. s., 2 H) 6.45-6.53 (m, 2 H) 6.59 (d, J=7.47 Hz, 1 H) 6.94 (d, J=5.27 Hz, 1 H) 7.22 (d, J=7.03 Hz, 1 H) 7.74 (d, J=5.27 Hz, 1 H) 8.40 (s, 1 H) 12.34 (br. s., 1 H). ¹³ C NMR (100 MHz, CDCl₃ ) δ ppm 26.36, 45.96, 56.82, 95.05, 115.71, 121.63, 125.85, 127.29, 127.62, 128.84, 131.20, 131.46, 135.66, 139.55, 140.06, 148.50 . m/z (ESI-MS) [M-tBu-amine]* 241.1 (100 %) [M]⁺ 314.1 (10 %).

### 5-(2-(3-((tert-Butylamino)methyl)phenyl)-1 H-pyrrolo[2,3 -b]pyridin-4 -yl)-1 H-indazol-3 -amine SU1547

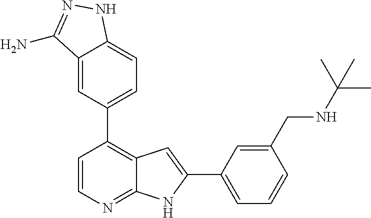 [SMILES: CC(C)(C)NCc1cccc(-c2cc3c(-c4ccc5[nH]nc(N)c5c4)ccnc3[nH]2)c1]

N-[3-(4 -Chloro-7 -azaindole)benzyl]-N-(tert-butyl)amine (0.14 g, 0.46 mmol), 5-(4,4,5,5 -tetramethyl-1,3,2 -dioxaborolan-2 -yl)-1 H-indazol-3 -amine (0.14 g, 0.55 mmol), 2 M potassium carbonate solution (0.46 ml, 0.92 mmol), [1,1 ′-bis(di-tert-butylphosphino)ferrocene]dichloropalladium(II) catalyst (0.015 g, 0.023 mmol) in 2 ml 1,4 dioxane were reacted as described in general procedure B and chromatographic purification (0 -10 % methanol in EtOAc), further purification by HPLC gave the titled compound as a yellow solid (40.8 mg, 21 %). ¹H NMR (400 MHz, DMSO-d₆ ) δ ppm 1.14 (s, 9 H) 3.43 (br. s., 2 H) 5.57 (s, 2 H) 7.13-7.24 (m, 2 H) 7.31-7.46 (m, 3 H) 7.72 (d, J=7.91 Hz, 1 H ) 6.84 (d, J=6.15 Hz, 1H) 7.96 (s, 1 H) 8.12-8.34 (m, 2 H) 12.21 (br. s., 1 H). ¹³ C NMR (100 MHz, DMSO-d₆ ) b ppm 29.06, 46.73, 60.32, 97.35, 109.66, 110.54, 115.07, 115.18, 119.11, 120.90, 124.28, 125.88, 127.27, 128.35, 129.21, 131.99, 139.00, 141.60, 141.75, 143.74, 149.17, 150.43, 151.02 . m/z (ESI-HRMS) calculated for C₂₅H₂₇N_{6=411.2292} found=411.2290 . (M+H)⁺.

### Route to SU1550

### N-[3-(4 -Chloro-7 -azaindole)benzyl]-N-cyclopentylamine

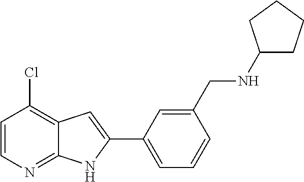 [SMILES: Clc1ccnc2[nH]c(-c3cccc(CNC4CCCC4)c3)cc12]

3-(4 -Chloro-7 -azaindol-2 -yl)benzaldehyde (0.12 g, 0.46 mmol) was reacted with cyclopentylamine (0.06 ml, 0.6 mmol), sodium triacetoxyborohydride (0.14 g, 0.69 mmol) and acetic acid (0.036 ml, 0.59 mmol) as described in general procedure A and chromatographic purification (0-10 % methanol in EtOAc) afforded the desired compound as a white solid (0.1 g, 68 %), ¹H NMR (400 MHz, DMSO-d₆ ) δ ppm 1.37 (m, 2 H) 1.46 (m, 2 H) 1.64 (m, 2 H) 1.73 (m, 2 H) 3.03 (m, 1 H) 3.74 (s, 2 H) 6.99 (s, 1 H) 7.21 (d, J=5.27 Hz, 1 H) 7.35 (d, J=7.91 Hz, 1 H) 7.41 (t, J=7.69 Hz, 1 H) 7.84 (d, J=7.47 Hz, 1 H) 7.97 (s, 1 H) 8.17 (d, J=5.27 Hz, 1 H) 12.49 (br. s., 1 H). m/z (ESI-MS) [M-cyclopentylamine]* 241.1 (100 %) [M]⁺ 326.2 (50 %).

### 5-(2-(3-((cyclopentylamino)methyl)phenyl)-1 H-pyrrolo[2,3 -b]pyridin-4 -yl)-1 H-indazol-3 -amine SU1550

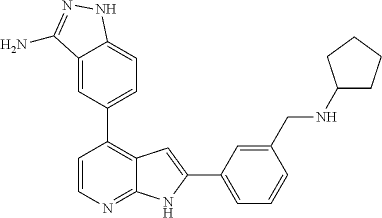 [SMILES: Nc1n[nH]c2ccc(-c3ccnc4[nH]c(-c5cccc(CNC6CCCC6)c5)cc34)cc12]

N-[3-(4 -Chloro-7 -azaindole)benzyl]-N-cyclopentylamine (0.1 g, 0.31 mmol), 5-(4,4,5,5 -tetramethyl-1,3,2 -dioxaborolan-2 -yl)-1 H-indazol-3 -amine (0.1 g, 0.38 mmol), 2 M potassium carbonate solution (0.31 ml, 0.63 mmol), [1,1 ′-bis(di-tert-butylphosphino)ferrocene]dichloropalladium(II) catalyst (0.01 g, 0.015 mmol) in 1 ml 1,4 dioxane were reacted as described in general procedure B and chromatographic purification (0 -18 % methanol in EtOAc and 1 % triethylamine) gave the titled compound as a brown solid (11.2 mg, 8 %), ¹H NMR (400 MHz, DMSO-d₆ ) δ ppm 1.38 (m, 2 H) 1.46 (m, 2 H) 1.64 (m, 2 H) 1.73 (m, 2 H) 3.04 (m, 1 H) 3.74 (s, 2 H) 5.56 (s, 2 H) 7.15-7.21 (m, 2 H) 7.32 (d, J=7.47 Hz, 1 H) 7.37-7.44 (m, 2 H) 7.72 (d, J=8.79 Hz, 1 H) 7.84 (d, J=7.47 Hz, 1 H) 7.94 (s, 1 H) 8.21-8.28 (m, 2 H) 12.20 (br. s., 1 H). ¹³ C NMR (100 MHz, DMSO-d₆ ) δ ppm 24.18, 33.02, 52.20, 59.11, 97.35, 109.70, 110.53, 115.05, 115.07, 119.11, 120.91, 124.17, 125.70, 127.27, 128.35, 129.18, 131.98, 139.06, 141.75, 141.65, 143.71, 147.82, 150.43, 151.02 m/z (ESI-HRMS) calculated for C₂₆H₂₇N_{6=423.2292} found=423.2289 . (M+H)⁺.

### Route to SU1548

### N-[3-(4 -Chloro-7 -azaindole)benzyl]-N-cyclohexylamine

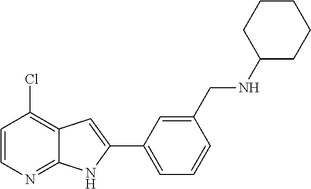 [SMILES: Clc1ccnc2[nH]c(-c3cccc(CNC4CCCCC4)c3)cc12]

3-(4 -Chloro-7 -azaindol-2 -yl)benzaldehyde (0.05 g, 0.19 mmol) was reacted with cyclohexylamine (0.03 ml, 0.25 mmol), sodium triacetoxyborohydride (0.06 g, 0.28 mmol) and acetic acid (0.014 ml, 0.25 mmol) as described in general procedure A and chromatographic purification (0-10 % methanol in EtOAc) afforded the desired compound as a pale yellow solid (0.04 g, 62 %), ¹H NMR (400 MHz, CDCl₃ ) b ppm 1.09-1.30 (m, 4 H) 1.62 (m, 2 H) 1.74 (m, 2 H) 1.91-1.99 (m, 2 H) 2.55 (m, 1 H) 3.89 (s, 2 H) 6.84 (s, 1 H) 7.08 (d, J=5.27 Hz, 1 H) 7.34 (d, J=7.47 Hz, 1 H) 7.42 (t, J=7.69 Hz, 1 H) 7.72 (d, J=7.91 Hz, 1 H) 7.82 (s, 1 H) 8.16 (d, J=5.27 Hz, 1 H). ¹³ C NMR (100 MHz, CDCl₃ ) δ ppm 25.10, 26.20, 33.52, 50.99, 56.64, 96.13, 116.39, 121.64, 124.56, 125.75, 128.60, 129.21, 131.84, 135.84, 140.00, 142.60, 150.21 . m/z (ESI-MS) [M]⁺ 340.1.

### 5-(2-(3-((Cyclohexylamino)methyl)phenyl)-1 H-pyrrolo[2,3 -b]pyridin-4 -yl)-1 H-indazol-3 -amine SU1548

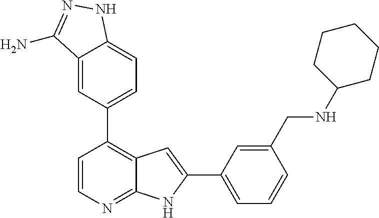 [SMILES: Nc1n[nH]c2ccc(-c3ccnc4[nH]c(-c5cccc(CNC6CCCCC6)c5)cc34)cc12]

N-[3-(4 -Chloro-7 -azaindole)benzyl]-N-cyclohexylamine (0.04 g, 0.11 mmol), 5-(4,4,5,5 -tetramethyl-1,3,2 -dioxaborolan-2 -yl)-1 H-indazol-3 -amine (0.036 g, 0.14 mmol), 2 M potassium carbonate solution (0.11 ml, 0.22 mmol), [1,1 ′-bis(di-tert-butylphosphino)ferrocene]dichloropalladium(II) catalyst (0.0035 g, 0.0055 mmol) in 0.6 ml 1,4 dioxane were reacted as described in general procedure B and chromatographic purification (0 -18 % methanol in EtOAc and 1 % triethylamine) gave the titled compound as a brown solid (19.3 mg, 40 %), ¹H NMR (400 MHz, DMSO-d₆ ) b ppm 1.05-1.25 (m, 4 H) 1.53 (m, 1 H) 1.66 (m, 2 H) 1.87 (m, 2 H) 3.79 (s, 2 H) 5.56 (s, 2 H) 7.15-7.17 (m, 1 H) 7.19 (d, J=5.27 Hz, 1 H) 7.31 -7.7.35 (m, 1 H) 7.37-7.43 (m, 2 H) 7.72 (dd, J=8.57, 1.54 Hz, 1 H) 7.83 (d, J=7.47 Hz, 1 H) 7.83 (d, J=7.47 Hz, 1 H) 7.93 (s, 1 H) 8.23 (s, 1 H) 8.26 (d, J=5.27 Hz, 1 H) 11.57 (s, 1 H) 12.19 (s, 1 H). ¹³ C NMR (100 MHz, DMSO-d₆ ) δ ppm 25.01, 26.49, 33.44, 50.51, 55.89, 97.34, 110.52, 115.05, 115.18, 116.06, 119.11, 120.90, 124.11, 125.57, 127.27, 128.35, 129.18, 131.97, 139.08, 141.57, 141.73, 141.73, 143.71, 143.71 . m/z (ESI-HRMS)[(ESI-HRMS) calculated for C₂₇H₂₉N_{6'}₂ 437.2448 found=437.2445 . (M+H)⁺.

### Route to SU1552

### N-[3-(4 -Chloro-7 -azaindole)benzyl]-N-butylamine

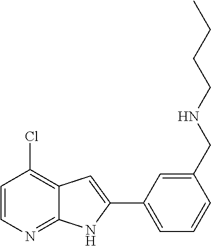 [SMILES: CCCCNCc1cccc(-c2cc3c(Cl)ccnc3[nH]2)c1]

3-(4 -Chloro-7 -azaindol-2 -yl)benzaldehyde (0.12 g, 0.46 mmol) was reacted with n-butylamine (0.06 ml, 0.46 mmol), sodium triacetoxyborohydride (0.14 g, 0.7 mmol) and acetic acid (0.03 ml, 0.6 mmol) as described in general procedure A and chromatographic purification (60-100 % EtOAc in petroleum ether 60-80 %) afforded the desired compound as a white solid (75 mg, 52 %).¹ H NMR (400 MHz, CDCl₃ ) δ ppm 0.91 (t, J=7.25 Hz, 3 H) 1.36 (dd, J=14.94, 7.47 Hz, 2 H) 1.54 (dt, J=14.83, 7.31 Hz, 2 H) 2.70 (t, J=7.25 Hz, 2 H) 3.89 (s, 2 H) 6.86 (s, 1 H) 7.10 (d, J=5.27 Hz, 1 H) 7.33 (d, J=7.47 Hz, 1 H) 7.42 (t, J=7.69 Hz, 1 H) 7.71 (d, J=7.47 Hz, 1 H) 7.82 (s, 1 H) 8.17 (d, J=5.27 Hz, 1 H). m/z (ESI-MS) [M]~314.1.

### 5-(2-(3-((Butylamino)methyl)phenyl)-1 H-pyrrolo[2,3 -b]pyridin-4 -yl)-1 H-indazol-3 -amine SU1552

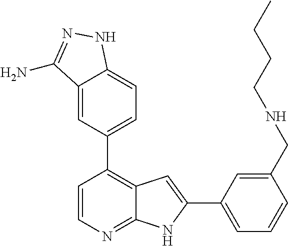 [SMILES: CCCCNCc1cccc(-c2cc3c(-c4ccc5[nH]nc(N)c5c4)ccnc3[nH]2)c1]

N-[3-(4 -Chloro-7 -azaindole)benzyl]-N-butylamine (0.07 g, 0.22 mmol), 5-(4,4,5,5 -tetramethyl-1,3,2 -dioxaborolan-2 -yl)-1 H-indazol-3 -amine (0.07 g, 0.26 mmol), 2 M potassium carbonate solution (0.22 ml, 0.44 mmol), [1,1 ′-bis(di-tert-butylphosphino)ferrocene]dichloropalladium(II) catalyst (0.007 g, 0.011 mmol) in 0.66 ml 1,4 dioxane were reacted as described in general procedure B and chromatographic purification (80-100 % EtOAc and 1 % triethylamine in petroleum ether 60-80 %) gave the titled compound as a dark brown solid (34.5 mg, 38 %), ¹ H NMR (400 MHz, DMSO-dc) 6 ppm 0.82-0.89 (m, 3 H) 1.27-1.36 (m, 2 H) 1.37-1.47 (m, 2 H) 1.89 (s, 1 H) 3.74 (s, 2 H) 5.56 (br. s., 2 H) 7.14-7.22 (m, 2 H) 7.29-7.35 (m, 1 H) 7.37-7.44 (m, 2 H) 7.72 (d, J=8.79 Hz, 1 H) 7.84 (d, J=7.91 Hz, 1 H) 7.93 (s, 1 H) 8.21-8.28 (m, 2 H) 11.57 (br. s., 1 H) 12.20 (br. s., 1 H). ³ C NMR (100 MHz, DMSO-d₆ ) δ ppm 14.41, 20.44, 13.84, 48.72, 53.27, 97.29, 110.44, 114.97, 115.80, 119.01, 120.83, 124.20, 125.66, 127.18, 128.18, 128.25, 129.15, 131.93, 138.92, 141.49, 141.57, 141.65, 143.64, 150.36, 150.93 . m/z (ESI-HRMS) calculated for C₂₅H₂₇N_{6'}₂ 411.2292 found=411.2289 . (M+H)⁺.

### Route to SU1553

### N-[3-(4 -Chloro-7 -azaindole)benzyl]-N-isopentylamine

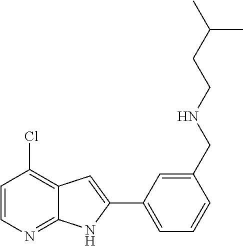 [SMILES: CC(C)CCNCc1cccc(-c2cc3c(Cl)ccnc3[nH]2)c1]

3-(4 -Chloro-7 -azaindol-2 -yl)benzaldehyde (0.07 g, 0.27 mmol) was reacted with isopentylamine (0.04 ml, 0.35 mmol), sodium triacetoxyborohydride (0.084 g, 0.4 mmol) and acetic acid (0.03 ml, 0.35 mmol) as described in general procedure A and chromatographic purification (50 % EtOAc in petroleum ether 60-80 %) afforded the desired compound as a yellowish oil (60.4 mg, 68 %), ¹H NMR (400 MHz, CDCl₃ ) δ ppm 0.89 (d, J=6.59 Hz, 6 H) 1.38-1.51 (m, 1 H), 1.64 (dt, J=13.51, 6.65 Hz, 2 H) 2.65-2.78 (m, 2 H) 3.89 (s, 2 H) 6.85 (s, 1 H) 7.09 (d, J=5.27 Hz, 1 H) 7.32 (d, J=7.47 Hz, 1 H) 7.41 (t, J=7.69 Hz, 1 H) 7.67 (d, J=7.47 Hz, 1 H) 7.80 (s, 1 H) 8.15 (d, J=5.27 Hz, 1 H). ¹³ C NMR (100 MHz, DMSO-d₆ ) δ ppm 21.65, 22.73, 26.25, 35.29, 38.13, 100.00, 116.60, 121.42, 124.59, 127.39, 129.25, 131.73, 134.60, 137.60, 139.81, 141.92, 143.71, 149.99 . .m/z (ESI-MS) [M]⁺ 328.2.

### 5-(2-(3-((Isopentylamino)methyl)phenyl)-1 H-pyrrolo[2,3 -b]pyridin-4 -yl)-1 H-indazol-3 -amine SU1553

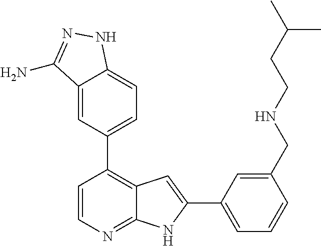 [SMILES: CC(C)CCNCc1cccc(-c2cc3c(-c4ccc5[nH]nc(N)c5c4)ccnc3[nH]2)c1]

N-[3-(4 -Chloro-7 -azaindole)benzyl]-N- isopentylamine (0.026 g, 0.08 mmol), 5-(4,4,5,5 -tetramethyl-1,3,2 -dioxaborolan-2 -yl)-1 H-indazol-3 -amine (0.025 g, 0.097 mmol), 2 M potassium carbonate solution (0.08 ml, 0.16 mmol), [1,1 ′-bis(di-tert-butylphosphino)ferrocene]dichloropalladium(II) catalyst (0.0026 g, 0.004 mmol) in 0.24 ml 1,4 dioxane were reacted as described in general procedure B and chromatographic purification (0 -15 % methanol in EtOAc and 1 % triethylamine), further purification by HPLC gave the titled compound as a yellow solid (15 mg, 44 %), ¹H NMR (500 MHz, DMSO-d6 ) δ ppm 0.84 (d, J=6.59 Hz, 6 H) 1.34 (q, J=7.01 Hz, 2 H) 1.60-1.66 (m, 1 H) 2.51-2.57 (m, 2 H) 3.75 (s, 2 H) 5.53 (s, 2 H) 7.15 (d, J=2.20 Hz, 1 H) 7.18 (d, J=5.02 Hz, 1 H) 7.31 (d, J=7.54 Hz, 1 H) 7.38 -7.42 (m, 2 H) 7.71 (dd, J=8.79, 1.57 Hz, 1 H) 7.83 (d, J=8.48 Hz, 1 H) 7.93 (s, 1 H) 8.22 (s, 1 H) 8.25 (d, J=5.02 Hz, 1 H) 11.54 (s, 1 H) 12.14 (s, 1 H)¹³ C NMR (100 MHz, DMSO-d₆ ) δ ppm 22.62, 25.81, 34.56, 45.77, 50.54, 97.75, 110.85, 114.68, 115.22, 119.81, 121.17, 126.09, 127.53, 128.18, 128.81, 129.71, 129.89, 132.42, 133.28, 138.34, 141.77, 141.89, 143.67, 149.38, 150.53 . m/z (ESI-HRMS) calculated for C₂₆H₂₉N_{6'}₂ 425.2448 found=425.2444 . (M+H)⁺.

### Route to SU1551

### N-[3-(4 -Chloro-7 -azaindole)benzyl]-N-butyl-N-ethylamine

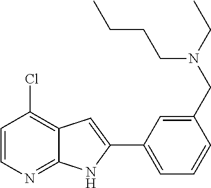 [SMILES: CCCCN(CC)Cc1cccc(-c2cc3c(Cl)ccnc3[nH]2)c1]

3-(4 -Chloro-7 -azaindol-2 -yl)benzaldehyde (0.12 g, 0.46 mmol) was reacted with butylethylamine (0.08 ml, 0.61 mmol), sodium triacetoxyborohydride (0.14 g, 0.7 mmol) and acetic acid (0.03 ml, 0.61 mmol) as described in general procedure A and chromatographic purification (40-60 % EtOAc in petroleum ether 60-80 %) afforded the desired compound as a white solid (78.3 mg, 50 %), ¹H NMR (400 MHz, CDCl₃ ) δ ppm 0.88 (t, J=7.47 Hz, 3 H) 1.06 (t, J=7.03 Hz, 3 H) 1.20-1.34 (m, 2 H) 1.42-152 (m, 2 H), 2.43-2.51 (m, 2 H) 2.57 (m, J=7.03, Hz, 2 H) 3.65 (s, 2 H) 6.88 (s, 1 H) 7.11 (d, J=5.27 Hz, 1 H) 7.39 (d, J=7.91 Hz, 1 H) 7.45 (t, J=7.69 Hz, 1 H) 7.72 (d, J=7.47 Hz, 1 H) 7.82 (s, 1 H) 8.22 (d, J=5.27 Hz, 1 H) 12.20 (br. s., 1 H) ).m/z (ESI-MS) [M]⁺ 342.2.

### 5-(2-(3-((Butyl(ethyl)amino)methyl)phenyl)-1 H-pyrrolo[2,3 -b]pyridin-4 -yl)-1 H-indazol-3 -amine SU1551

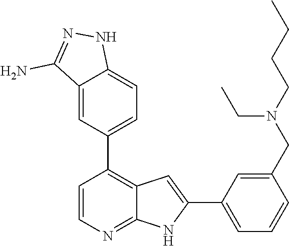 [SMILES: CCCCN(CC)Cc1cccc(-c2cc3c(-c4ccc5[nH]nc(N)c5c4)ccnc3[nH]2)c1]

N-[3-(4 -Chloro-7 -azaindole)benzyl]-N-butyl-N-ethylamine (0.051 g, 0.15 mmol), 5-(4,4,5,5 -tetramethyl-1,3,2 -dioxaborolan-2 -yl)-1 H-indazol-3 -amine (0.046 g, 0.18 mmol), 2 M potassium carbonate solution (0.15 ml, 0.3 mmol), [1,1 ′-bis(di-tert-butylphosphino)ferrocene]dichloropalladium(II) catalyst (0.0048 g, 0.007 mmol) in 0.4 ml 1,4 dioxane were reacted as described in general procedure B and chromatographic purification (80-100 % EtOAc and 1 % triethylamine in petroleum ether 60-80 %) gave the titled compound as a dark brown solid (27 mg, 41 %), ¹H NMR (400 MHz DMSO-d₆ ) δ ppm 0.82 (t, J=7.25 Hz, 3 H) 1.00 (t, J=7.03 Hz, 3 H) 1.13-1.17 (m, 2 H) 1.27 (m, 4 H) 3.58 (s, 2 H) 5.56 (br. s., 2 H) 7.14 (d, J=1.76 Hz, 1 H) 7.19 (d, J=5.27 Hz, 1 H) 7.31 (d, J=7.03 Hz, 1 H) 7.40 (d, J=8.35 Hz, 2 H) 7.72 (dd, J=8.79, 1.76 Hz, 1 H) 7.85 (d, J=7.47 Hz, 1 H) 7.89 (s, 1 H) 8.23 (s, 1 H) 8.26 (d, J=4.83 Hz, 1 H) 11.56 (s., 1 H) 12.23 (s., 1 H). ¹³ C NMR (100 MHz, DMSO-d₆ ) δ ppm 12.02, 14.36, 20.50, 29.08, 47.19, 52.63, 57.88, 97.24, 110.43, 114.96, 115.06, 118.99, 120.82, 124.32, 126.11, 127.18, 128.25, 128.68, 129.13, 131.85, 138.93, 141.19, 141.48, 141.65, 143.64, 150.35, 150.93 . m/z (ESI-HRMS) calculated for C₂₇H₃₁N₆=439.2605 found=439.2601 . (M+H)⁺.

### Route to SU1546

### N-[3-(4 -Chloro-7 -azaindole)benzyl]-N,N-dibutylamine

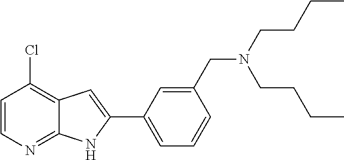 [SMILES: CCCCN(CCCC)Cc1cccc(-c2cc3c(Cl)ccnc3[nH]2)c1]

3-(4 -Chloro-7 -azaindol-2 -yl)benzaldehyde (0.16 g, 0.66 mmol) was reacted with dibutylamine (0.14 ml, 0.85 mmol), sodium triacetoxyborohydride (0.21 g, 0.99 mmol) and acetic acid (0.05 ml, 0.85 mmol) as described in general procedure A and chromatographic purification (50 % EtOAc in petroleum ether 60-80 %) afforded the desired compound as a white solid (0.09 g, 53 %), ¹H NMR (400 MHz, DMSO-d₆ ) δ ppm 0.88 (t, J=7.25 Hz, 6 H) 1.24-1.36 (m, 4 H) 1.43 -1.52 (m, 4 H) 2.42-2.52 (t, J=7.25 Hz, 4 H) 3.64 (s, 2 H) 6.86 (s, 1 H) 7.10 (d, J=5.27 Hz, 1 H) 7.38-7.48 (m, 2 H) 7.75 (d, J=7.47 Hz, 1 H) 7.83 (s, 1 H) 8.20 (d, J=5.27 Hz, 1 H) 13.07 (br. s., 1 H). ¹³ C NMR (100 MHz, DMSO-d₆ ) δ ppm 14.44, 20.57, 21.33, 29.29, 53.45, 58.50, 60.32, 95.40, 116.42, 120.48, 124.54, 126.50, 129.32, 131.16, 133.99, 135.90, 140.11, 141.68, 143.90, 150.70 . m/z (ESI-MS) [M]⁺ 370.1.

### 5-(2-(3-((Dibutylamino)methyl)phenyl)-1 H-pyrrolo[2,3 -b]pyridin-4 -yl)-1 H-indazol-3 -amine SU1546

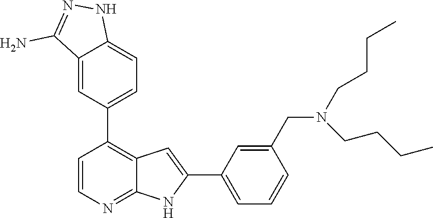 [SMILES: CCCCN(CCCC)Cc1cccc(-c2cc3c(-c4ccc5[nH]nc(N)c5c4)ccnc3[nH]2)c1]

N-[3-(4 -chloro-7 -azaindole)benzyl]-N,N-dibutylamine (0.095 g, 0.24 mmol), 5-(4,4,5,5 -tetramethyl-1,3,2 -dioxaborolan-2 -yl)-1 H-indazol-3 -amine (0.075 g, 0.29 mmol) ), 2 M potassium carbonate solution (0.24 ml, 0.48 mmol), [1,1 ′-bis(di-tert-butylphosphino)ferrocene]dichloropalladium(II) catalyst (0.007 g, 0.012 mmol) in 1 ml 1,4 dioxane were reacted as described in general procedure B and chromatographic purification (30-100 % EtOAc and 1 % triethylamine in petroleum ether 60-80 %), further purification by HPLC gave the titled compound as a yellow solid (38 mg, 34 %), ¹H NMR (400 MHz, DMSO-d₆ ) δ ppm 0.82 (t, J=7.03 Hz, 6 H) 1.21-1.31 (m, 4 H) 1.36-1.47 (m, 4 H) 2.39 (t, J=6.59 Hz, 4 H) 3.57 (s, 2 H) 5.56 (br. s., 2 H) 7.14 (br. s., 1 H) 7.19 (d, J=4.83 Hz, 1 H) 7.30 (d, J=7.03 Hz, 1 H) 7.37-7.44 (m, 2 H) 7.72 (d, J=8.35 Hz, 1 H) 7.82-7.90 (m, 2 H) 8.21-8.28 (m, 2 H) 11.57 (br. s., 1 H) 12.24 (br. s., 1 H). ¹³ C NMR (100 MHz, DMSO-d₆ ) δ ppm 14.46, 20.58, 29.27, 53.45, 58.60, 97.29, 110.48, 115.05, 115.19, 118.07, 119.09, 120.93, 124.36, 126.20, 127.23, 128.34, 128.74, 129.20, 131.92, 139.04, 141.58, 141.75, 143.73, 150.45, 151.05 . m/z (ESI-HRMS) calculated for C₂9H₃₅N₆=467.2918 found=467.2917 . (M+H)⁺.

### 5-(2-(2 -Fluoro-6-(piperidin-1 -ylmethyl)pyridin-4 -yl)-1 H-pyrrolo[2,3 -b]pyridin-4 -yl)-1 H-indazol-3 -amine SU1386

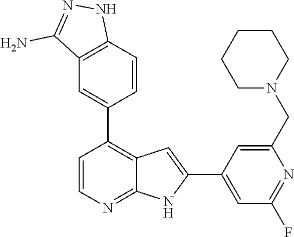 [SMILES: Nc1n[nH]c2ccc(-c3ccnc4[nH]c(-c5cc(F)nc(CN6CCCCC6)c5)cc34)cc12]

A 2-5 mL Biotage MW tube was charged with methyl 4 -chloro-2-(2 -fluoro-6-(piperidin-1 -ylmethyl)pyridin-4 -yl)-1 H-pyrrolo[2,3 -b]pyridine (100 mg, 0.3 mmol), 5-(4,4,5,5 -tetramethyl-1,3,2 -dioxaborolan-2 -yl)-1 H-indazol-3 -amine (103 mg, 0.4 mmol, 1.3 eq.), [1,1 ′-bis(di-tert-butylphosphino)ferrocene]dichloropalladium(II) catalyst (9.8 mg, 0.015 mmol, 5 mol %) and dioxane (1.8 mL). The resulting suspension was purged with nitrogen for 5 minutes before adding 1 M aq. K₃ PO₄ (1.8 mL). The reaction mixture was heated to 110° C. for 16 hrs. The reaction mixture was then allowed to cool to room temperature, diluted with water (10 mL) and extracted with EtOAc (3×20 mL). The combined organic fractions were then dried over anhydrous magnesium sulfate, filtered and the solvent removed under reduced pressure. The crude product was then purified by HPLC to give the desired product as a pale yellow solid (37 mg). ¹H NMR (500 MHz, DMSO-D₆ ) δ 1.39-1.53 (m, 6 H), 2.41 (m, 4 H), 3.52 (s, 2 H), 5.54 (s, 2 H), 7.24 (d, J=4.7 Hz, 1 H), 7.41 (d, J=8.5 Hz, 1 H), 7.52 (s, 1 H), 7.62 (m, 1 H), 7.73 (d, J=8.4 Hz, 1 H), 7.91 (s, 1 H), 8.28 (s, 1 H), 8.36 (d, J=5.6 Hz, 1 H), 12.55 (s, 1 H). LRMS: Calculated for C₂₅H₂₄FN₇ 441.21 ; Found:442.27 (M+H)⁺.

### tert-Butyl 4-((4-(4-(3 -amino-1 H-indazol-5 -yl)-1 H-pyrrolo[2,3 -b]pyridin-2 -yl)-6 -fluoropyridin-2 -yl)methyl)piperazine-1 -carboxylate SU1388

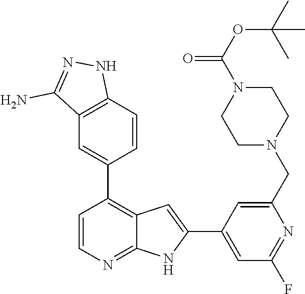 [SMILES: CC(C)(C)OC(=O)N1CCN(Cc2cc(-c3cc4c(-c5ccc6[nH]nc(N)c6c5)ccnc4[nH]3)cc(F)n2)CC1]

A 2-5 mL Biotage MW tube was charged with tert-butyl 4-((4-(4 -chloro-1 H-pyrrolo[2,3 -b]pyridin-2 -yl)-6 -fluoropyridin-2 -yl)methyl)piperazine-1 -carboxylate (100 mg, 0.3 mmol), 5-(4,4,5,5 -tetramethyl-1,3,2 -dioxaborolan-2 -yl)-1 H-indazol-3 -amine (103 mg, 0.4 mmol, 1.3 eq.), [1,1 ′-bis(di-tert-butylphosphino)ferrocene]dichloropalladium(II) catalyst (9.8 mg, 0.015 mmol, 5 mol %) and dioxane (1.8 mL). The resulting suspension was purged with nitrogen for 5 minutes before adding 1 M aq. K₃ PO₄ (1.8 mL). The reaction mixture was heated to 110° C. for 16 hrs. The reaction mixture was then allowed to cool to room temperature, diluted with water (10 mL) and extracted with EtOAc (3×20 mL). The combined organic fractions were then dried over anhydrous magnesium sulfate, filtered and the solvent removed under reduced pressure. The crude product was then purified by HPLC to give the desired product as a pale yellow solid (34 mg). ¹H NMR (500 MHz, DMSO-D₆ ) δ 1.39 (s, 9 H), 2.44 (m, 4 H), 3.37 (m, 4 H), 3.60 (s, 2 H), 5.55 (s, 2 H), 7.27 (d, J=4.7 Hz, 1 H), 7.43 (d, J=8.5 Hz, 1 H), 7.55 (s, 1 H), 7.66 (s, 1 H), 7.73 (d, J=8.4 Hz, 1 H), 7.95 (s, 1 H), 8.25 (s, 1 H), 8.38 (d, J=5.6 Hz, 1 H), 11.59 (s, 1 H), 12.55 (s, 1 H). LRMS: Calculated for C₂₉H₃₁FN₈O₂ 542.26 ; Found:543.04

### 5-(2-(2 -Fluoro-6-(piperazin-1 -ylmethyl)pyridin-4 -yl)-1 H-pyrrolo[2,3 -b]pyridin-4 -yl)-1 H-indazol-3 -amine SU1390

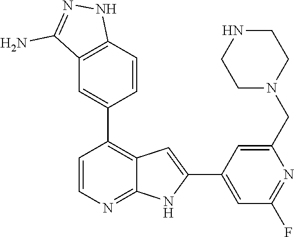 [SMILES: Nc1n[nH]c2ccc(-c3ccnc4[nH]c(-c5cc(F)nc(CN6CCNCC6)c5)cc34)cc12]

A 2-5 mL Biotage MW tube was charged with tert-butyl 4-((4-(4-(3 -amino-1 H-indazol-5 -yl)-1 H-pyrrolo[2,3 -b]pyridin-2 -yl)-6 -fluoropyridin-2 -yl)methyl)piperazine-1 -carboxylate (SU1388 ) (100 mg, 0.3 mmol), and THF (5 mL). The resulting suspension was purged with nitrogen for 5 minutes before adding TFA (0.5 mL). The solution was allowed to stir for 16 hrs before being diluted with water (10 mL) and extracted with EtOAc (3×20 mL). The combined organic fractions were then dried over anhydrous magnesium sulfate, filtered and the solvent removed under reduced pressure. The crude product was then purified by HPLC to give the desired product as a pale yellow solid (18 mg). ¹H NMR (500 MHz, DMSO-D₆ ) δ 3.18 (m, 6 H), 3.80 (m, 4 H), 7.27 (d, J=4.7 Hz, 1 H), 7.46 (d, J=8.5 Hz, 1 H), 7.55 (s, 1 H), 7.70 (s, 1 H), 7.77 (dd, J=8.4, 1.6 Hz, 1 H), 7.96 (s, 1 H), 8.28 (s, 1 H), 8.39 (d, J=5.6 Hz, 1 H), 12.60 (s, 1 H). LRMS: Calculated for C₂₄H₂₃FN₈ 442.20 ; Found:443.27 . (M+H)⁺.

### 5-(2-(2 -Fluoro-6-((4 -methylpiperazin-1 -yl)methyl)pyridin-4 -yl)-1 H-pyrrolo[2,3 -b]pyridin-4 -yl)-1 H-indazol-3 -amine SU1383

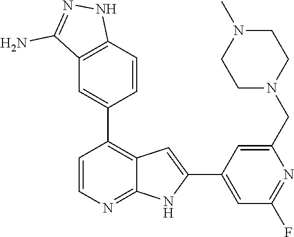 [SMILES: CN1CCN(Cc2cc(-c3cc4c(-c5ccc6[nH]nc(N)c6c5)ccnc4[nH]3)cc(F)n2)CC1]

A 2-5 mL Biotage MW tube was charged with 4 -chloro-2-(2 -fluoro-6-(piperazin-1 -ylmethyl)pyridin-4 -yl)-1 H-pyrrolo[2,3 -b]pyridine (100 mg, 0.3 mmol), 5-(4,4,5,5 -tetramethyl-1,3,2 -dioxaborolan-2 -yl)-1 H-indazol-3 -amine (103 mg, 0.4 mmol, 1.3 eq.), [1,1 -bis(di-tert-butylphosphino)ferrocene]dichloropalladium(II) catalyst (9.8 mg, 0.015 mmol, 5 mol %) and dioxane (1.8 mL). The resulting suspension was purged with nitrogen for 5 minutes before adding 1 M aq. K₃ PO₄ (1.8 mL). The reaction mixture was heated to 110° C. for 16 hrs. The reaction mixture was then allowed to cool to room temperature, diluted with water (10 mL) and extracted with EtOAc (3×20 mL). The combined organic fractions were then dried over anhydrous magnesium sulfate, filtered and the solvent removed under reduced pressure. The crude product was then purified by HPLC to give the desired product as a pale yellow solid (18 mg). ¹H NMR (500 MHz, DMSO-D₆ ) δ 2.79 (s, 3 H), 3.08 (m, 4 H), 3.40 (m, 4 H), 3.70 (m, 4 H), 7.27 (d, J=4.7 Hz, 1 H), 7.46 (d, J=8.5 Hz, 1 H), 7.54 (s, 1 H), 7.68 (s, 1 H), 7.77 (d, J=8.4 Hz, 1 H), 7.93 (s, 1 H), 8.27 (s, 1 H), 8.38 (d, J=5.6 Hz, 1 H), 12.55 (s, 1 H). LRMS: Calculated for C₂₅H₂₅FNₛ 456.22 ; Found:457.20 (M+H)⁺.

### 5-(2-(2-((tert-Butylamino)methyl)-6 -fluoropyridin-4 -yl)-1 H-pyrrolo[2,3 -b]pyridin-4 -yl)-1 H-indazol-3 -amine SU1387

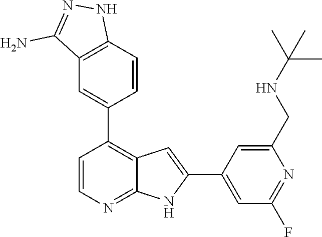 [SMILES: CC(C)(C)NCc1cc(-c2cc3c(-c4ccc5[nH]nc(N)c5c4)ccnc3[nH]2)cc(F)n1]

A 2-5 mL Biotage MW tube was charged with N-((4-(4 -chloro-1 H-pyrrolo[2,3 -b]pyridin-2 -yl)-6 -fluoropyridin-2 -yl)methyl)-2 -methylpropan-2 -amine (100 mg, 0.3 mmol), 5-(4,4,5,5 -tetramethyl-1,3,2 -dioxaborolan-2 -yl)-1 H-indazol-3 -amine (103 mg, 0.4 mmol, 1.3 eq.), [1,1 ′-bis(di-tert-butylphosphino)ferrocene]dichloropalladium(II) catalyst (9.8 mg, 0.015 mmol, 5 mol %) and dioxane (1.8 mL). The resulting suspension was purged with nitrogen for 5 minutes before adding 1 M aq. K₃ PO₄ (1.8 mL). The reaction mixture was heated to 110° C. for 16 hrs. The reaction mixture was then allowed to cool to room temperature, diluted with water (10 mL) and extracted with EtOAc (3×20 mL). The combined organic fractions were then dried over anhydrous magnesium sulfate, filtered and the solvent removed under reduced pressure. The crude product was then purified by HPLC to give the desired product as a pale yellow solid (28 mg). ¹H NMR (500 MHz, DMSO-D₆ ) δ 1.15 (s, 9 H), 3.76 (s, 2 H), 5.55 (s, 2 H), 7.26 (d, J=4.7 Hz, 1 H), 7.43 (d, J=8.5 Hz, 1 H), 7.54 (s, 1 H), 7.61 (s, 1 H), 7.73 (d, J=8.4 Hz, 1 H), 7.95 (s, 1 H), 8.27 (s, 1 H), 8.38 (d, J=5.6 Hz, 1 H), 11.60 (s, 1 H), 12.55 (s, 1 H). LRMS: Calculated for C₂₄H₂₄FN₇ 429.21 ; Found:430.30 (M+H)⁺.

### 5-(2-(2-((Cyclohexylamino)methyl)-6 -fluoropyridin-4 -yl)-1 H-pyrrolo[2,3 -b]pyridin-4 -yl)-1 H-indazol-3 -amine SU1395

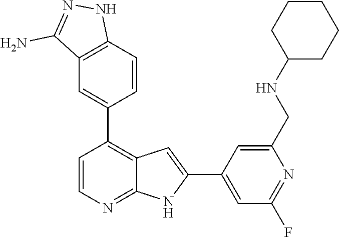 [SMILES: Nc1n[nH]c2ccc(-c3ccnc4[nH]c(-c5cc(F)nc(CNC6CCCCC6)c5)cc34)cc12]

A 2-5 mL Biotage MW tube was charged with N-((4-(4 -chloro-1 H-pyrrolo[2,3 -b]pyridin-2 -yl)-6 -fluoropyridin-2 -yl)methyl)cyclohexanamine (100 mg, 0.3 mmol), 5-(4,4,5,5 -tetramethyl-1,3,2 -dioxaborolan-2 -yl)-1 H-indazol-3 -amine (103 mg, 0.4 mmol, 1.3 eq.), [1,1 ′-bis(di-tert-butylphosphino)ferrocene]dichloropalladium(II) catalyst (9.8 mg, 0.015 mmol, 5 mol %) and dioxane (1.8 mL). The resulting suspension was purged with nitrogen for 5 minutes before adding 1 M aq. K₃ PO₄ (1.8 mL). The reaction mixture was heated to 110° C. for 16 hrs. The reaction mixture was then allowed to cool to room temperature, diluted with water (10 mL) and extracted with EtOAc (3×20 mL). The combined organic fractions were then dried over anhydrous magnesium sulfate, filtered and the solvent removed under reduced pressure. The crude product was then purified by HPLC to give the desired product as a pale yellow solid (23 mg). ¹H NMR (500 MHz, DMSO-D₆ ) δ 1.25 (m, 6 H), 1.65 (s, 1 H), 1.80 (m, 2 H), 2.15 (m, 2 H), 3.15 (m, 1 H), 4.35 (m, 3 H), 7.30 (d, J=4.7 Hz, 1 H), 7.49 (d, J=8.5 Hz, 1 H), 7.58 (s, 1 H), 7.79 (d, J=8.4 Hz, 1 H), 7.86 (s, 1 H), 8.10 (s, 1 H), 8.29 (s, 1 H), 8.42 (d, J=5.6 Hz, 1 H), 9.05 (s, 1 H), 12.55 (s, 1 H). LRMS: Calculated for C₂₆H₂₆FN₇ 455.22 ; Found:456.13 (M+H)⁺.

### 5-(2-(2 -Fluoro-6-((phenylamino)methyl)pyridin-4 -yl)-1 H-pyrrolo[2,3 -b]pyridin-4 -yl)-1 H-indazol-3 -amine SU1394

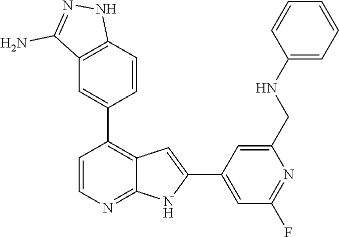 [SMILES: Nc1n[nH]c2ccc(-c3ccnc4[nH]c(-c5cc(F)nc(CNc6ccccc6)c5)cc34)cc12]

A 2-5 mL Biotage MW tube was charged with N-((4-(4 -chloro-1 H-pyrrolo[2,3 -b]pyridin-2 -yl)-6 -fluoropyridin-2 -yl)methyl)aniline (100 mg, 0.3 mmol), 5-(4,4,5,5 -tetramethyl-1,3,2 -dioxaborolan-2 -yl)-1 H-indazol-3 -amine (103 mg, 0.4 mmol, 1.3 eq.), [1,1 ′-bis(di-tert-butylphosphino)ferrocene]dichloropalladium(II) catalyst (9.8 mg, 0.015 mmol, 5 mol %) and dioxane (1.8 mL). The resulting suspension was purged with nitrogen for 5 minutes before adding 1 M aq. K₃ PO₄ (1.8 mL). The reaction mixture was heated to 110° C. for 16 hrs. The reaction mixture was then allowed to cool to room temperature, diluted with water (10 mL) and extracted with EtOAc (3×20 mL). The combined organic fractions were then dried over anhydrous magnesium sulfate, filtered and the solvent removed under reduced pressure. The crude product was then purified by HPLC to give the desired product as a pale yellow solid (52 mg). ¹H NMR (500 MHz, DMSO-D₆ ) δ 4.37 (s, 2 H), 6.59 (m, 3 H), 7.07 (t, J=8.5 Hz, 1 H), 7.17 (s, 1 H), 7.26 (d, J=8.5 Hz, 1 H), 7.49 (d, J=8.5 Hz, 1 H), 7.51 (d, J=8.5 Hz, 1 H), 7.80 (d, J=8.4 Hz, 1 H), 8.29 (s, 1 H), 8.35 (d, J=5.6 Hz, 1 H), 8.49 (t, J=5.6 Hz, 1 H), 12.38 (s, 1 H).

LRMS: Calculated for C₂₆H₂₀FN₇ 449.18 ; Found:450.20 (M+H)⁺.

### 4-(4-(3 -Amino-1 H-indazol-5 -yl)-1 H-pyrrolo[2,3 -b]pyridin-2 -yl)-2,6 -difluorobenzamide SU1370

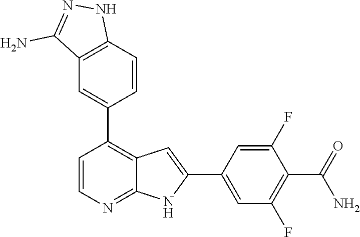 [SMILES: NC(=O)c1c(F)cc(-c2cc3c(-c4ccc5[nH]nc(N)c5c4)ccnc3[nH]2)cc1F]

A 2-5 mL Biotage MW tube was charged with 4-(4 -chloro-1 H-pyrrolo[2,3 -b]pyridin-2 -yl)-2,6 -difluorobenzamide (123 mg, 0.4 mmol, 1 eq.), 5-(4,4,5,5 -tetramethyl-1,3,2 -dioxaborolan-2 -yl)-1 H-indazol-3 -amine (135 mg, 0.52 mmol, 1.3 eq.), [1,1 ′-bis(di-tert-butylphosphino)ferrocene]dichloropalladium(II) catalyst (13 mg, 0.02 mmol, 5 mol %) and ethanol (1.6 mL, oxygen free). The resulting suspension was purged with nitrogen for 10 minutes before adding 1 M aq. K₃ PO₄ (0.8 mL, oxygen free). The reaction was heated to 50° C. under a gentle flow of nitrogen for 10 minutes, then at 100° C. for 24 hours. The stirred reaction mixture was then cooled to room temperature and slowly diluted with water (8 mL) to precipitate a solid, which was filtered and washed with water (8 mL×2 ), and hexane (7 mL×2 ). The filtered solid was sonicated with 30 % MeOH in AcOEt (15 mL) and filtered. The filtrate was then cooled to 4° C. to give the title compounds as a beige solid (16 mg). ¹H NMR (400 MHz, DMSO-D₆ ) δ 5.56 (s, 2 H), 7.25 (d, J=5.0 Hz, 1 H), 7.40 (d, J=8.8 Hz, 1 H), 7.46 (s, 1 H), 7.74 (d, J=8.6 Hz, 1 H), 7.80-7.90 (m, 3 H), 8.16 (s, 1 H), 8.23 (s, 1 H), 8.32 (d, J=5.1 Hz, 1 H), 11.59 (s, 1 H), 12.38 (s, 1 H).

### Route to SU1554

### N-[4-(4 -chloro-7 -azaindole)]-2,6 -difluorobenzyl]-N,N-dimethylamine

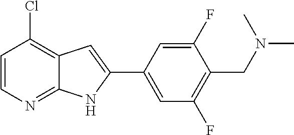 [SMILES: CN(C)Cc1c(F)cc(-c2cc3c(Cl)ccnc3[nH]2)cc1F]

4-(4 -Chloro-7 -azaindol-2 -yl)-2,6 -difluorobenzaldehyde (0.08 g, 0.27 mmol) was reacted with dimethylamine (0.17 ml, 0.35 mmol), sodium triacetoxyborohydride (0.086 g, 0.41 mmol) and acetic acid (0.02 ml, 0.35 mmol) as described in general procedure A and chromatographic purification (0-5 % EtOAc in petroleum ether 60-80 %) afforded the desired compound as a white solid (40 mg, 46 %). ¹H NMR (400 MHz, DMSO-d₆ ) δ ppm 2.17 (s, 6 H) 3.52 (s, 2 H) 7.23-7.25 (m, 2 H) 7.79 (s, 1 H) 7.82 (s, 1 H) 8.22 (d, J=5.27 Hz, 1 H) 12.59 (s, 1 H).

### 5-(2-(4-((Dimethylamino)methyl)-3,5 -difluorophenyl)-1 H-pyrrolo[2,3 -b]pyridin-4 -yl)-1 H-indazol-3 -amine SU1554

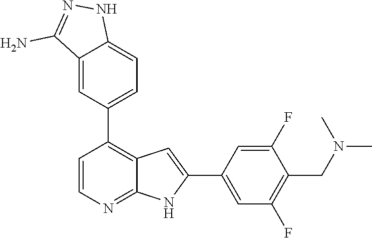 [SMILES: CN(C)Cc1c(F)cc(-c2cc3c(-c4ccc5[nH]nc(N)c5c4)ccnc3[nH]2)cc1F]

N-[4-(4 -Chloro-7 -azaindole)]-2,6 -difluorobenzyl]-N,N-dimethylamine (0.04 g, 0.12 mmol), 5-(4,4,5,5 -tetramethyl-1,3,2 -dioxaborolan-2 -yl)-1 H-indazol-3 -amine (0.038 g, 0.15 mmol), 2 M potassium carbonate solution (0.12 ml, 0.24 mmol), [1,1 ′-bis(di-tert-butylphosphino)ferrocene]dichloropalladium(II) catalyst (0.004 g, 0.006 mmol) in 0.37 ml 1,4 dioxane were reacted as described in general procedure B and chromatographic purification (0-10 % methanol in EtOAc and 1 % triethylamine), further purification by HPLC gave the titled compound as a yellow solid (23.8 mg, 47 %), ¹H NMR (400 MHz, DMSO-d₆ ) δ ppm 2.17 (s, 6 H) 3.52 (s, 2 H) 5.57 (s, 2 H) 7.24 (d, J=5.27 Hz, 1 H) 7.38-7.42 (m, 2 H) 7.74 (dd, J=8.57, 1.54 Hz, 1 H) 7.79 (d, J=9.23 Hz, 2 H) 8.24 (s, 1 H) 8.31 (d, J=5.27 Hz, 1 H) 11.58 (s, 1 H) 12.33 (s, 1 H). ¹³ C NMR 42.62, 47.75, 100.72, 108.80, 109.05, 111.06, 111.66, 112.01, 115.37, 118.68, 121.36, 121.60, 128.67, 129.68, 130.55, 135.67, 138.09, 141.89, 142.32, 145.05, 150.83. ¹⁹ F NMR (DMSO-d₆ ) -110.44 . m/z (ESI-HRMS) calculated for C₂₃H₂₁N₆ F₂=419.1790 found=419.1786 . (M+H)⁺.

### 5-(2-(3,5 -Difluoro-4-(piperidin-1 -ylmethyl)phenyl)-1 H-pyrrolo[2,3 -b]pyridin-4 -yl)-1 H-indazol-3 -amine SU1382

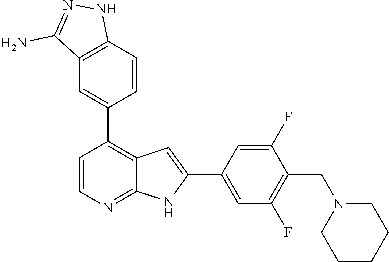 [SMILES: Nc1n[nH]c2ccc(-c3ccnc4[nH]c(-c5cc(F)c(CN6CCCCC6)c(F)c5)cc34)cc12]

A 0-5-2 mL Biotage MW tube was charged with 4 -chloro-2-(3,5 -difluoro-4-(piperidin-1 -ylmethyl)phenyl)-1 H-pyrrolo[2,3 -b]pyridine (69 mg, 0.190 mmol, 1 eq.), 5-(4,4,5,5 -tetramethyl-1,3,2 -dioxaborolan-2 -yl)-1 H-indazol-3 -amine (64 mg, 0.247 mmol, 1.3 eq.), [1,1 ′-bis(di-tert-butylphosphino)ferrocene]dichloropalladium(II) catalyst (6.2 mg, 0.0095 mmol, 5 mol %) and ethanol (0.76 mL, oxygen free). The resulting suspension was purged with nitrogen for 10 minutes before adding 1 M aq. K₃ PO₄ (0.38 mL, oxygen free). The reaction was heated to 50° C. under a gentle flow of nitrogen for 10 minutes, then to 110° C. for 19 hours. The stirred reaction mixture was then cooled to room temperature and slowly diluted with water (7 mL) and filtered. The filtered solid was washed with 1 % aq. NH₃ (3 mL×3 ), and dried in the vacuum oven to give the title compound as a beige solid (73 mg, 84 %). ¹H NMR (400 MHz, DMSO-D₆ ) δ 1.30-1.38 (m, 2 H), 1.44-1.51 (m, 4 H), 2.34-2.41 (m, 4 H), 3.55 (s, 2 H), 5.56 (s, 2 H), 7.23 (d, J=5.0 Hz, 1 H), 7.37-7.43 (m, 2 H), 7.71-7.81 (m, 3 H), 8.23 (s, 1 H), 8.30 (d, J=5.1 Hz, 1 H), 11.58 (s, 1 H), 12.33 (s, 1 H).

### Route to SU1555

### N-[4-(4 -Chloro-7 -azaindole)]-2,6 -difluorobenzyl]-N-isopropyl-N-methylamine

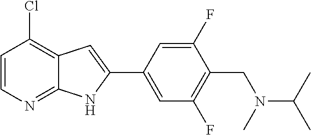 [SMILES: CC(C)N(C)Cc1c(F)cc(-c2cc3c(Cl)ccnc3[nH]2)cc1F]

4-(4 -Chloro-7 -azaindol-2 -yl)-2,6 -difluorobenzaldehyde (0.12 g, 0.41 mmol) was reacted with isopropylmethylamine (0.05 ml, 0.53 mmol), sodium triacetoxyborohydride (0.13 g, 0.61 mmol) and acetic acid (0.03 ml, 0.53 mmol) as described in general procedure A and chromatographic purification (10-32 % EtOAc in petroleum ether 60-80 %) afforded the desired compound as a white solid (110 mg, 76 %), ¹H NMR (400 MHz, DMSO-d₆ ) δ ppm 1.02 (d, J=6.15 Hz, 6 H) 2.09 (s, 3 H) 2.85 (dt, J=12.85, 6.54 Hz, 1 H) 3.33 (s, 2 H) 7.18-7.27 (m, 2 H) 7.77 (d, J=8.35 Hz, 2 H) 8.22 (d, J=5.27 Hz, 1 H) 12.59 (br. s., 1 H). ¹³ C NMR (100 MHz, DMSO-d₆ ) δ ppm 18.09, 36.79, 44.16, 53.58, 97.56, 108.58, 108.95, 115.40, 115.60, 116.62, 132.86, 134.63, 137.28, 144.79, 150.55, 160.98, 161.08, 163.42, 163.52. ¹⁹ F NMR (DMSO-d₆ ) -114.08 . m/z (ESI-MS) [M]⁺ 350.1.

### 5-(2-(3,5 -Difluoro-4-((isopropyl(methyl)amino)methyl)phenyl)-1 H-pyrrolo[2,3 -b]pyridin-4 -yl)-1 H-indazol-3 -amine SU1555

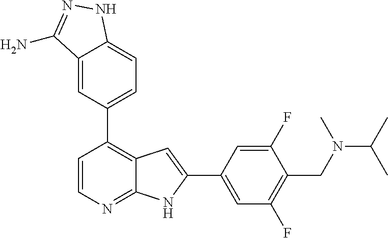 [SMILES: CC(C)N(C)Cc1c(F)cc(-c2cc3c(-c4ccc5[nH]nc(N)c5c4)ccnc3[nH]2)cc1F]

N-[4-(4 -Chloro-7 -azaindole)]-2,6 -difluorobenzyl]- N-isopropyl-N-methylamine (0.05 g, 0.14 mmol), 5-(4,4,5,5 -tetramethyl-1,3,2 -dioxaborolan-2 -yl)-1 H-indazol-3 -amine (0.044 g, 0.17 mmol), 2 M potassium carbonate solution (0.14 ml, 0.28 mmol), [1,1 ′-bis(di-tert-butylphosphino)ferrocene]dichloropalladium(II) catalyst (0.004 g, 0.007 mmol) in 0.4 ml 1,4 dioxane were reacted as described in general procedure B and chromatographic purification (60-98 % EtOAc and 1 % triethylamine in petroleum ether 60-80 %), further purification by HPLC gave the titled compound as a yellow solid (19.5 mg, 31 %), ¹H NMR (500 MHz, DMSO-d₆ ) δ ppm 1.02 (d, J=6.59 Hz, 6 H) 2.10 (s, 3 H) 2.85 (quin, J=6.51 Hz, 1 H) 3.53 (s, 2 H) 5.53 (s, 2 H) 7.22 (d, J=5.02 Hz, 1 H) 7.37 (s, 1 H) 7.40 (d, J=8.48 Hz, 1 H) 7.71-7.76 (m, 3 H) 8.22 (s, 1 H) 8.29 (d, J=5.02 Hz, 1 H) 11.56 (s, 1 H) 12.29 (br. s., 1 H). ¹³ C NMR (100 MHz, DMSO-d₆ ) δ ppm 18.11, 36.75, 44.20, 53.53, 99.47, 108.35, 108.62, 110.46, 109.26, 115.13, 116.62, 118.65, 120.92, 127.20, 127.89, 133.47, 133.62, 135.59, 140.63, 142.10, 144.63, 146.84, 150.97. ⁴⁹ F NMR (DMSO-d₆ ) -110.56 . m/z (ESI-HRMS) calculated for C₂₅H₂₅N₆ F₂=447.2103 found=447.2099 . (M+H)⁺.

### Route to SU1556

### N-[4-(4 -Chloro-7 -azaindole)]-2,6 -difluorobenzyl]-N-butyl-N-ethylamine

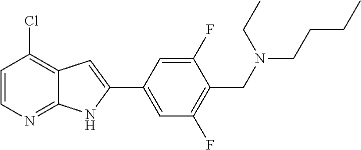 [SMILES: CCCCN(CC)Cc1c(F)cc(-c2cc3c(Cl)ccnc3[nH]2)cc1F]

4-(4 -Chloro-7 -azaindol-2 -yl)-2,6 -difluorobenzaldehyde (0.12 g, 0.41 mmol) was reacted with butylethylamine (0.07 ml, 0.53 mmol), sodium triacetoxyborohydride (0.13 g, 0.61 mmol) and acetic acid (0.03 ml, 0.53 mmol) as described in general procedure A and chromatographic purification (0-10 % EtOAc in petroleum ether 60-80 %) afforded the desired compound as a white solid (30 mg, 19 %). ¹H NMR (400 MHz, DMSO-d₆ ) δ ppm 0.83 (t, J=7.47 Hz, 3 H) 0.99 (t, J=7.03 Hz, 3 H) 1.18-1.30 (m, 2 H) 1.40 (quin, J=7.14 Hz, 2 H) 2.38 (t, J=7.03 Hz, 2 H) 2.45 (d, J=7.47 Hz, 2 H) 3.60 (s, 2 H) 7.20-7.26 (m, 2 H) 7.78 (d, J=8.79 Hz, 2 H) 8.22 (d, J=5.27 Hz, 1 H) 12.60 (br. s., 1 H). ¹³ C NMR (100 MHz, DMSO-d₆ ) δ ppm 11.47, 14.17 20.21, 28.07, 42.39, 46.46, 51.04, 97.72, 108.79, 109.07, 115.07, 116.65, 120.08, 133.33, 134,69, 136.59, 144.87, 150.58. ¹⁹ F NMR (DMSO-d₆ ) -115.34 . m/z (ESI-MS) [M]⁻ 376.0

### 5-(2-(4-((Butyl(ethyl)amino)methyl)-3,5 -difluorophenyl)-1 H-pyrrolo[2,3 -b]pyridin-4 -yl)-1 H-indazol-3 -amine SU1556

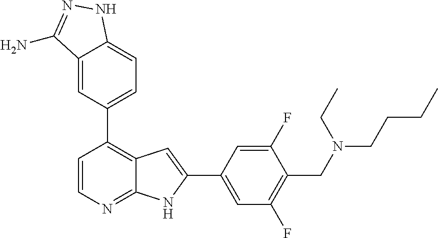 [SMILES: CCCCN(CC)Cc1c(F)cc(-c2cc3c(-c4ccc5[nH]nc(N)c5c4)ccnc3[nH]2)cc1F]

N-[4-(4 -Chloro-7 -azaindole)]-2,6 -difluorobenzyl]-N-butyl-N-ethylamine (0.026 g, 0.068 mmol), 5-(4,4,5,5 -tetramethyl-1,3,2 -dioxaborolan-2 -yl)-1 H-indazol-3 -amine (0.021 g, 0.08 mmol), 2 M potassium carbonate solution (0.068 ml, 0.13 mmol), [1,1 ′-bis(di-tert-butylphosphino)ferrocene]dichloropalladium(II) catalyst (0.002 g, 0.0034 mmol) in 0.2 ml 1,4 dioxane were reacted as described in general procedure B and purification by HPLC gave the titled compound as a yellow solid (20 mg, 36 %), ¹H NMR (400 MHz, DMSO-d,) δ ppm 0.94 (t, J=7.40 Hz, 3 H) 1.30 (t, J=7.15 Hz, 3 H) 1.33-140 (m, 2 H) 1.71 (t, J=7.40 Hz, 2 H) 4.43 (s, 2 H) 7.27 (d, J=5.02 Hz, 1 H) 7.44 (d, J=8.53 Hz, 1 H) 7.52 (d, J=2.01 Hz, 1 H) 7.77 (dd, J=8.78 Hz, 1 H) 7.97 (d, J=9.54 Hz, 2 H) 8.23-8.26 (m, 1 H) 8.36 (d, J=5.02 Hz, 1 H) 11.68 (br. s, 1 H) 12.44 (s., 1 H). ¹⁹ F NMR (DMSO-d₆ ) -110.42 . m/z (ESI-HRMS) calculated for C₂₇H₂₉N₆ F₂=475.2416 found=475.2414 . (M+H)⁺.

### 5-(2-(4-((Dibutylamino)methyl)-3,5 -difluorophenyl)-1 H-pyrrolo[2,3 -b]pyridin-4 -yl)-1 H-indazol-3 -amine SU1393

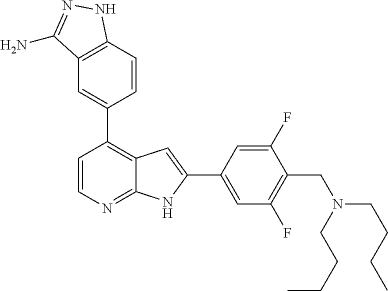 [SMILES: CCCCN(CCCC)Cc1c(F)cc(-c2cc3c(-c4ccc5[nH]nc(N)c5c4)ccnc3[nH]2)cc1F]

A 0-5-2 mL Biotage MW tube was charged with N-butyl-N-(4-(4 -chloro-1 H-pyrrolo[2,3 -b]pyridin-2 -yl)-2,6 -difluorobenzyl)butan-1 -amine (81 mg, 0.200 mmol, 1 eq.), 5-(4,4,5,5 -tetramethyl-1,3,2 -dioxaborolan-2 -yl)-1 H-indazol-3 -amine (67 mg, 0.260 mmol, 1.3 eq.), [1,1 ′-bis(di-tert-butylphosphino)ferrocene]dichloropalladium(li) catalyst (6.5 mg, 0.01 mmol, 5 mol %) and ethanol (0.8 mL, oxygen free). The resulting suspension was purged with nitrogen for 10minutes before adding 1 M aq. K₃ PO₄ (0.4 mL, oxygen free). The reaction was heated to 50° C. under a gentle flow of nitrogen for 10 minutes, then to 110° C. for 19 hours. The stirred reaction mixture was then cooled to room temperature and slowly diluted with water (7 mL) and filtered. The filtered solid was washed with water (3 mL×3 ) and hexane (3 mL×3 ), and purified by flash chromatography (SiO₂, 50 g, 0-4 % MeOH in AcOEt containing 1 % triethylamine) to give the title compound as a yellow solid (39 mg, 39 %).¹ H NMR (400 MHz, DMSO-D₆ ) δ 0.82 (t, J=7.3 Hz, 6 H), 1.25 (h, J=7.3 Hz, 4 H), 1.41 (p, J=7.6, 7.2 Hz, 4 H), 2.37 (t, J=7.0 Hz, 4 H), 3.30 (s, 1 H), 3.60 (s, 2 H), 5.55 (d, J=8.9 Hz, 1 H), 7.23 (d, J=5.0 Hz, 1 H), 7.37-7.44 (m, 2 H), 7.71-7.80 (m, 3 H), 8.23 (s, 1 H), 8.30 (d, J=5.0 Hz, 1 H), 11.58 (s, 1 H), 12.31 (s, 1 H).

### Two Substituents: One in Position 7 of the Aminoindazole Moiety; One in Position 2 of the Azaindole Moiety

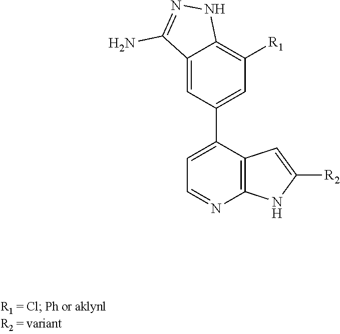

### 3 -Chloro-2 -fluoro-5-(4,4,5,5 -tetramethyl-1,3,2 -dioxaborolan-2 -yl)benzonitrile

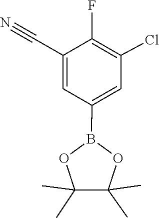 [SMILES: CC1(C)OB(c2cc(Cl)c(F)c(C#N)c2)OC1(C)C]

Preparation of stock solution: [Ir(OMe)cod]₂ ([(1,5 -cyclooctadiene) (methoxy) iridium (1 ) dimer]) (104 mg, 0.312 mmol), dtbpy (di-tert-butyl-2,2 ′dipyridyl) (84 mg, 0.312 mmol) and B₂ pin₂ (bis (pinacolato) diboron) (2644 mg, 10.4 mmol) were mixed in volumetric flask and diluted up to 25 ml with MTBE (tert-butyl-methylether). Then 12.6 ml of stock solution was added to 3 -chloro-2 -fluorobenzonitrile (0.85 g, 5.5 mmol) in microwave vial under nitrogen. The reaction vial then deoxygenated, sealed and heated to 80° C. for 30 min in microwave. Thereafter, the solvent was evaporated, and residue purified by column chromatography (0-20 % EtOAc in petroleum ether 60-80 %) to give the title compound as a white solid (1.3 g, 84 %). ¹H NMR (400 MHz, DMSO-d₆ ) δ ppm 1.31 (s, 12 H) 7.99-8.07 (m, 2 H).¹³ C NMR (100 MHz, DMSO-d₆ ) δ ppm 25.05, 85.32, 102.93, 113.26, 121.36, 136.54, 138.60, 141.51, 159.40.

### 7 -Chloro-5-(4,4,5,5 -tetramethyl-1,3,2 -dioxaborolan-2 -yl)-1 H-indazol-3 -amine

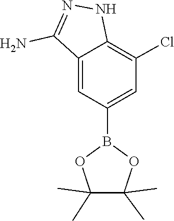 [SMILES: CC1(C)OB(c2cc(Cl)c3[nH]nc(N)c3c2)OC1(C)C]

To a solution of 3 -chloro-2 -fluoro-5-(4,4,5,5 -tetramethyl-1,3,2 -dioxaborolan-2 -yl) benzonitrile (187 ) (0.4 g, 1.4 mmol) in ethanol (20 ml), hydrazine hydrate (0.22 ml, 2.47 g, 6.8 mmol, 50 -60 %) was added and the reaction refluxed for 18 h. The solvent was evaporated, and the residue was triturated with 12 ml EtOAc and petroleum ether (1:1 ) and then filtrated under vacuum and washed with water and petroleum ether 60-80 % to give the desired compound as a yellow solid (320 mg, 78 %). ¹H NMR (400 MHz, DMSO-d₆ ) δ ppm 1.31 (s, 12 H) 5.68 (s, 2 H) 7.45 (s, 1 H) 8.15 (s, 1 H) 12.02 (br. s., 1 H). ¹³ C NMR (100 MHz, DMSO-d₆ ) δ ppm 25.17, 84.07, 102.89, 116.70, 119.82, 125.89, 128.07, 130.59, 146.33.

### General Procedure A:

A suspension of required chloroaryl (1 eq.), 5-(4,4,5,5 -tetramethyl-1,3,2 -dioxaborolan-2 -yl)-1 H-indazol-3 -amine (1.2-1.5 eq.), base (2 eq.) in 1:3 of solvent was deoxygenated with nitrogen in sealed tube. Then [1,1 ′-bis(di-tert-butylphosphino)ferrocene]dichloropalladium(II) catalyst (0.05 eq.) was added then the tube was sealed and the mixture allowed to stirred at 90-100° C. for 18 h. After the reaction was cooled to room temperature, EtOAc and water were added. Extracted organic layer was dried over magnesium sulfate and concentrated under reduced pressure and the residue purified by column chromatography.

### 7 -Chloro-5-(2 -phenyl-1 H-pyrrolo[2,3 -b]pyridin-4 -yl)-1 H-indazol-3 -amine SU1406

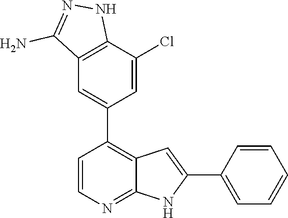 [SMILES: Nc1n[nH]c2c(Cl)cc(-c3ccnc4[nH]c(-c5ccccc5)cc34)cc12]

A 2-5 mL Biotage MW tube was charged with 4 -chloro-2 -phenyl-1 H-pyrrolo[2,3 -b]pyridine (100 mg, 0.3 mmol), 7 -chloro-5-(4,4,5,5 -tetramethyl-1,3,2 -dioxaborolan-2 -yl)-1 H-indazol-3 -amine (111 mg, 0.4 mmol, 1.3 eq.), [1,1 ′-bis(di-tert-butylphosphino)ferrocene]dichloropalladium(II) catalyst (9.8 mg, 0.015 mmol, 5 mol %) and dioxane (1.8 mL). The resulting suspension was purged with nitrogen for 5 minutes before adding 1 M aq. K₃ PO₄ (1.8 mL). The reaction mixture was heated to 110° C. for 16 hrs. The reaction mixture was then allowed to cool to room temperature, diluted with water (10 mL) and extracted with EtOAc (3×20 mL). The combined organic fractions were then dried over anhydrous magnesium sulfate, filtered and the solvent removed under reduced pressure. The crude product was then purified by HPLC to give the desired product as a pale yellow solid (52 mg). ¹H NMR (500 MHz, DMSO-D₆ ) δ 5.73 (s, 2 H), 7.17 (d, J=1.9 Hz, 1 H), 7.22 (d, J=8.5 Hz, 1 H), 7.35 (t, J=7.5 Hz, 1 H), 7.49 (t, J=7.9 Hz, 1 H), 7.74 (s, 1 H), 7.99 (d, J=8.5 Hz, 1 H), 8.25 (m, 2 H), 12.05 (s, 1 H), 12.27 (s, 1 H). LRMS: Calculated for C₂₀H₁₄ ClN₅ 359.09 ; Found:360.10 (M+H)⁺.

### 7 -Chloro-5-(2-(3-(morpholinomethyl)phenyl)-1 H-pyrrolo[2,3 -b]pyridin-4 -yl)-1 H-indazol-3 -amine SU1404

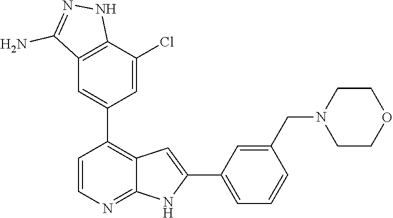 [SMILES: Nc1n[nH]c2c(Cl)cc(-c3ccnc4[nH]c(-c5cccc(CN6CCOCC6)c5)cc34)cc12]

A 2-5 mL Biotage MW tube was charged with 4-(3-(4 -chloro-1 H-pyrrolo[2,3 -b]pyridin-2 -yl)benzyl)morpholine (100 mg, 0.3 mmol), 7 -chloro-5-(4,4,5,5 -tetramethyl-1,3,2 -dioxaborolan-2 -yl)-1 H-indazol-3 -amine (111 mg, 0.4 mmol, 1.3 eq.), [1,1 ′-bis(di-tert-butylphosphino)ferrocene]dichloropalladium(II) catalyst (9.8 mg, 0.015 mmol, 5 mol %) and dioxane (1.8 mL). The resulting suspension was purged with nitrogen for 5 minutes before adding 1 M aq. K₃ PO₄ (1.8 mL). The reaction mixture was heated to 110° C. for 16 hrs. The reaction mixture was then allowed to cool to room temperature, diluted with water (10 mL) and extracted with EtOAc (3×20 mL). The combined organic fractions were then dried over anhydrous magnesium sulfate, filtered and the solvent removed under reduced pressure. The crude product was then purified by HPLC to give the desired product as a pale yellow solid (26 mg). ¹H NMR (500 MHz, DMSO-De) δ 2.39 (m, 4 H), 3.58 (t, J=4.4 Hz, 4 H), 5.73 (s, 2 H), 7.14 (d, J=1.9 Hz, 1 H), 7.21 (d, J=8.5 Hz, 1 H), 7.31 (d, J=7.5 Hz, 1 H), 7.43 (t, J=7.9 Hz, 1 H), 7.73 (s, 1 H), 7.99 (m, 2 H), 8.27 (m, 2 H), 12.05 (s, 1 H), 12.27 (s, 1 H). LRMS: Calculated for C₂₅H₂₃ ClN₆0 458.16 ; Found:459.20 (M+H)⁺.

### 7 -Chloro-5-(2-(4-(morpholinomethyl)phenyl)-1 H-pyrrolo[2,3 -b]pyridin-4 -yl)-1 H-indazol-3 -amine SU1405

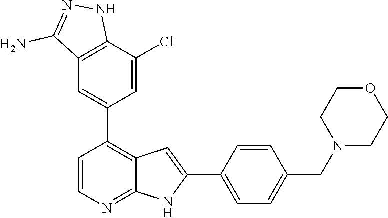 [SMILES: Nc1n[nH]c2c(Cl)cc(-c3ccnc4[nH]c(-c5ccc(CN6CCOCC6)cc5)cc34)cc12]

A 2-5 mL Biotage MW tube was charged with 4-(4-(4 -chloro-1 H-pyrrolo[2,3 -b]pyridin-2 -yl)benzyl)morpholine (100 mg, 0.3 mmol), 7 -chloro-5-(4,4,5,5 -tetramethyl-1,3,2 -dioxaborolan-2 -yl)-1 H-indazol-3 -amine (111 mg, 0.4 mmol, 1.3 eq.), [1,1 ′-bis(di-tert-butylphosphino)ferrocene]dichloropalladium(II) catalyst (9.8 mg, 0.015 mmol, 5 mol %) and dioxane (1.8 mL). The resulting suspension was purged with nitrogen for 5 minutes before adding 1 M aq. K₃ PO₄ (1.8 mL). The reaction mixture was heated to 110° C. for 16 hrs. The reaction mixture was then allowed to cool to room temperature, diluted with water (10 mL) and extracted with EtOAc (3×20 mL). The combined organic fractions were then dried over anhydrous magnesium sulfate, filtered and the solvent removed under reduced pressure. The crude product was then purified by HPLC to give the desired product as a pale yellow solid (18 mg). ¹H NMR (500 MHz, DMSO-De) δ 2.37 (m, 4 H), 3.58 (t, J=4.4 Hz, 4 H), 5.72 (s, 2 H), 7.14 (d, J=1.9 Hz, 1 H), 7.20 (d, J=8.5 Hz, 1 H), 7.41 (m, 2 H), 7.73 (s, 1 H), 7.95 (m, 2 H), 8.24 (m, 2 H), 12.05 (s, 1 H), 12.23 (s, 1 H). LRMS: Calculated for C₂₅H₂₃ ClN₆0 458.16 ; Found:459.20 (M+H)⁺.

### 7 -Chloro-5-(2-(2 -fluoro-6-(piperazin-1 -ylmethyl)pyridin-4 -yl)-1 H-pyrrolo[2,3 -b]pyridin-4 -yl)-1 H-indazol-3 -amine SU1401

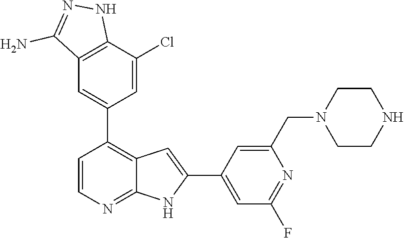 [SMILES: Nc1n[nH]c2c(Cl)cc(-c3ccnc4[nH]c(-c5cc(F)nc(CN6CCNCC6)c5)cc34)cc12]

A 2-5 mL Biotage MW tube was charged with tert-butyl 4-((4-(4 -chloro-1 H-pyrrolo[2,3 -b]pyridin-2 -yl)-6 -fluoropyridin-2 -yl)methyl)piperazine-1 -carboxylate (100 mg, 0.3 mmol), 7 -chloro-5-(4,4,5,5 -tetramethyl-1,3,2 -dioxaborolan-2 -yl)-1 H-indazol-3 -amine (111 mg, 0.4 mmol, 1.3 eq.), [1,1 ′-bis(di-tert-butylphosphino)ferrocene]dichloropalladium(II) catalyst (9.8 mg, 0.015 mmol, 5mol %) and dioxane (1.8 mL). The resulting suspension was purged with nitrogen for 5 minutes before adding 1 M aq. K₃ PO₄ (1.8 mL). The reaction mixture was heated to 110° C. for 16 hrs. The reaction mixture was then allowed to cool to room temperature, diluted with water (10 mL) and extracted with EtOAc (3×20 mL). The combined organic fractions were then dried over anhydrous magnesium sulfate, filtered and the solvent removed under reduced pressure. The crude product was then dissolved in dichloromethane (20 mL) and TFA (1 mL) added and the solution allowed to stir at RT for 16 hrs, the solvent was then removed under reduced pressure and the crude product purified by HPLC to give the desired product as a pale yellow solid (18 mg). ¹H NMR (500 MHz, DMSO-D₆ ) δ 2.80 (m, 4 H), 3.18 (m, 4 H), 3.79 (s, 2 H), 5.72 (s, 2 H), 7.29 (d, J=5.05 Hz, 1 H), 7.54 (d, J=1.7 Hz, 1 H), 7.71 (s, 1 H), 7.75 (d, J=1.25 Hz, 1 H), 7.96 (s, 1 H), 8.23 (d, J=1.30 Hz, 1 H), 8.58 (bs, 1 H), 12.61 (s, 1 H). LRMS: Calculated for C₂₂H₂₄ ClFN₈ 476.16 ; Found:477.27 . (M+H)⁺.

### 7 -Phenyl-5-(2 -phenyl-1 H-pyrrolo[2,3 -b]pyridin-4 -yl)-1 H-indazol-3 -amine SU1410

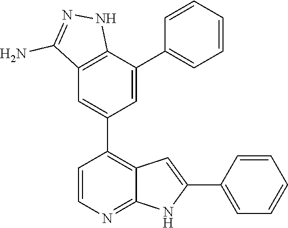 [SMILES: Nc1n[nH]c2c(-c3ccccc3)cc(-c3ccnc4[nH]c(-c5ccccc5)cc34)cc12]

A 2-5 mL Biotage MW tube was charged with 7 -Chloro-5-(2 -phenyl-1 H-pyrrolo[2,3 -b]pyridin-4 -yl)-1 H-indazol-3 -amine (SU1406 ) (100 mg, 0.3 mmol), phenylboronic acid (78 mg, 0.4 mmol, 1.3 eq.), [1,1 ′-bis(di-tert-butylphosphino)ferrocene]dichloropalladium(11 ) catalyst (9.8 mg, 0.015 mmol, 5 mol %) and dioxane (1.8 mL). The resulting suspension was purged with nitrogen for 5 minutes before adding 1 M aq. KaPO₄ (1.8 mL). The reaction mixture was heated to 110° C. for 16 hrs. The reaction mixture was then allowed to cool to room temperature, diluted with water (10 mL) and extracted with EtOAc (3×20 mL). The combined organic fractions were then dried over anhydrous magnesium sulfate, filtered and the solvent removed under reduced pressure. The crude product was then purified by HPLC to give the desired product as a pale yellow solid (52 mg). ¹H NMR (500 MHz, DMSO-D₆ ) δ 5.62 (s, 2 H), 7.20 (d, J=1.9 Hz, 1 H), 7.29 (d, J=8.5 Hz, 1 H), 7.36 (t, J=7.5 Hz, 1 H), 7.44-7.55 (m, 5 H), 7.70 (s, 1 H), 7.80 (d, J=8.5 Hz, 2 H), 7.80 (d, J=8.5 Hz, 2 H), 8.25 (m, 2 H), 11.07 (s, 1 H), 12.24 (s, 1 H). LRMS: Calculated for C₂₆H₁₉N_{5 401.16} ; Found:402.20 (M+H)⁺.

### 5-(2-(3-(Morpholinomethyl)phenyl)-1 H-pyrrolo[2,3 -b]pyridin-4 -yl)-7 -phenyl-1 H-indazol-3 -amine SU1409

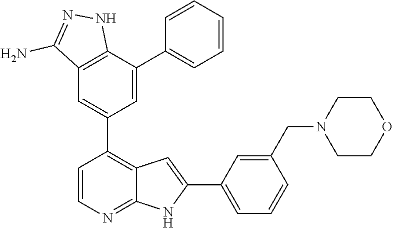 [SMILES: Nc1n[nH]c2c(-c3ccccc3)cc(-c3ccnc4[nH]c(-c5cccc(CN6CCOCC6)c5)cc34)cc12]

A 2-5 mL Biotage MW tube was charged with 7 -chloro-5-(2-(3-(morpholinomethyl)phenyl)-1 H-pyrrolo[2,3 -b]pyridin-4 -yl)-1 H-indazol-3 -amine (SU1404 ) (100 mg, 0.3 mmol), phenylboronic acid (78 mg, 0.4 mmol, 1.3 eq.), [1,1 ′-bis(di-tert-butylphosphino)ferrocene]dichloropalladium(II) catalyst (9.8 mg, 0.015 mmol, 5 mol %) and dioxane (1.8 mL). The resulting suspension was purged with nitrogen for 5 minutes before adding 1 M aq. K₃ PO₄ (1.8 mL). The reaction mixture was heated to 110° C. for 16 hrs. The reaction mixture was then allowed to cool to room temperature, diluted with water (10 mL) and extracted with EtOAc (3×20 mL). The combined organic fractions were then dried over anhydrous magnesium sulfate, filtered and the solvent removed under reduced pressure. The crude product was then purified by HPLC to give the desired product as a pale yellow solid (13 mg). ¹H NMR (500 MHz, DMSO-D₆ ) δ 2.39 (m, 4 H), 3.58 (m, 6 H), 5.63 (s, 2 H), 7.17 (d, J=1.9 Hz, 1 H), 7.29 (m, 2 H), 7.42 (m, 2 H), 7.53 (m, 2 H), 7.70 (s, 1 H), 7.80 (m, 2 H), 7.78 (m, 2 H), 8.27 (m, 2 H), 11.70 (s, 1 H), 12.34 (s, 1 H). LRMS: Calculated for C₃₁H₂₈N₆O 500.23 ; Found:501.20 (M+H)⁺.

### 5-(2-(4-(Morpholinomethyl)phenyl)-1 H-pyrrolo[2,3 -b]pyridin-4 -yl)-7 -phenyl-1 H-indazol-3 -amine

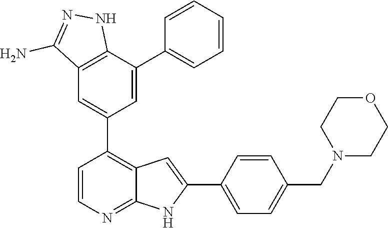 [SMILES: Nc1n[nH]c2c(-c3ccccc3)cc(-c3ccnc4[nH]c(-c5ccc(CN6CCOCC6)cc5)cc34)cc12]

A 2-5 mL Biotage MW tube was charged with 7 -Chloro-5-(2-(4-(morpholinomethyl)phenyl)-1 H-pyrrolo[2,3 -b]pyridin-4 -yl)-1 H-indazol-3 -amine (100 mg, 0.3 mmol), Phenylboronic acid (78 mg, 0.4 mmol, 1.3 eq.), [1,1 ′-bis(di-tert-butylphosphino)ferrocene]dichloropalladium(II) catalyst (9.8 mg, 0.015 mmol, 5 mol %) and dioxane (1.8 mL). The resulting suspension was purged with nitrogen for 5 minutes before adding 1 M aq. K₃ PO₄ (1.8 mL). The reaction mixture was heated to 110° C. for 16 hrs. The reaction mixture was then allowed to cool to room temperature, diluted with water (10 mL) and extracted with EtOAc (3×20 mL). The combined organic fractions were then dried over anhydrous magnesium sulfate, filtered and the solvent removed under reduced pressure the crude product was then purified by HPLC to give the desired product as a pale yellow solid (13 mg). ¹H NMR (500 MHz, DMSO-D₆ ) δ 2.36 (m, 4 H), 3.49 (s, 2 H) 3.57 (m, 4 H), 5.62 (s, 2 H), 7.17 (d, J=1.9 Hz, 1 H), 7.28 (d, J=5.05 Hz, 1 H), 7.42 (m, 3 H), 7.53 (m, 2 H), 7.70 (s, 1 H), 7.80 (m, 2 H), 7.78 (m, 2 H), 8.25 (m, 2 H), 11.70 (s, 1 H), 12.20 (s, 1 H). LRMS: Calculated for C₃₁H₂₈N₆O 500.23 ; Found:501.20 (M+H)⁺.

1. A compound, or a pharmaceutically acceptable salt or solvate thereof, having the structural formula (1 ) shown below: 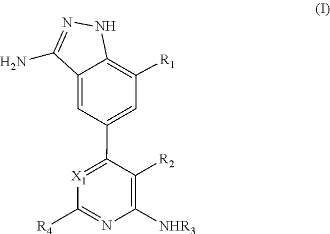
wherein:
R₁ is selected from hydrogen, halogen, (1-6 C)alkyl, (2-6 C)alkenyl, (2-6 C)alkynyl, (3 -7 C)cycloalkyl, aryl, heteroaryl and heterocyclyl,
wherein said (1-6 C)alkyl, (2-6 C)alkenyl, (2-6 C)alkynyl, (3-7 C)cycloalkyl, aryl, heteroaryl and heterocyclyl are optionally substituted by one or more R₁₀₀ substituents;
wherein each R₁₀₀ is independently selected from halo, (1-2 C)haloalkyl (e.g. trifluoromethyl), (1-2 C)haloalkoxy e.g. (trifluoromethoxy), cyano, hydroxyl, (1-4 C)alkyl, (1 -4 C)hydroxyalkyl, (CH₂ )ₓOR_{f}, (CH₂ )ₓ C(O)R_{f}, (CH₂ )ₓ C(O)OR_{f}, (CH₂ )ₓOC(O)R_{f}, (CH₂ )ₓ C(O)N(Rⱼ )Rₕ, (CH₂ )ₓ N(R_{g} )C(O)R_{f}, (CH₂ )ₓ S(O)_{y1} R_{f}, (CH₂ )ₓ SO₂ N(Rⱼ )Rₕ, (CH₂ )ₓ N(R_{g} )SO₂ R_{f}, (CH₂ )ₓ NRⱼ Rₕ, (CH₂ )ₓ (3-7 C)cycloalkyl, (CH₂ )ₓ heterocyclyl, (CH₂ )ₓ heteroaryl or (CH₂ )ₓ aryl;
and wherein:
(i) R_{f} and R_{g} are each independently selected from hydrogen, (1-6 C)alkyl or phenyl; and wherein Rₕ and Rⱼ are each independently selected from hydrogen, (1 -6 C)alkyl or phenyl or Rₕ and Rⱼ together with the nitrogen atom to which they are attached form a 3-7 membered ring which may optionally include further heteroatoms and is optionally further substituted by one or more substituents selected from halo, trifluoromethyl, trifluoromethoxy, cyano, hydroxyl, carboxyl, carbamoyl, sulphamoyl, and (1-2 C)alkyl; and
(ii) any (1-4 C)alkyl, (3-7 C)cycloalkyl, heterocyclyl, heteroaryl or aryl in a R₁₀₀ substituent group is optionally further substituted by one or more substituents selected from halo, trifluoromethyl, trifluoromethoxy, cyano, hydroxyl, (1 -2 C)alkyl, (1-2 C)haloalkyl, (1-2 C)hydroxyalkyl, ORₖ, C(O)Rₖ, C(O)ORₖ, OC(O)Rₖ, C(O)N(Rₗ )Rₖ, N(Rₗ )C(O)Rₖ, S(O)_{y2} Rₖ, SO₂ N(R)Rₖ, N(Rₗ )SO₂ Rₖ, or NRₗ Rₖ, wherein Rₖ and Rₗ are selected from hydrogen or (1-2 C)alkyl;
R₂ is hydrogen;
R₃ is selected from hydrogen, (1-8 C)alkyl, (2-8 C)alkenyl, (2-8 C)alkynyl, (3-7 C)cycloalkyl, aryl, heteroaryl, heterocyclyl, (CH₂ )₀₋₃ (3-7 C)cycloalkyl, (CH₂ )₀₋₃ heterocyclyl, (CH₂ )₀₋₃ heteroaryl, (CH₂ )₀₋₃ aryl, —C(O)-(CH₂ )₀₋₃ (3-7 C)cycloalkyl, —C(O)-(CH₂ )₀₋₃ heterocyclyl, —C(O)-(CH₂ )₀₋₃ heteroaryl, —C(O)-(CH₂ )₀₋₃ aryl or —C(O)O(1-8 C)alkyl, —C(O)NR₃ₐ-(1-8 C)alkyl, —C(O)NR₃ₐ-(CH₂ )₀₋₃ (3-7 C)cycloalkyl, —C(O)NR₃ₐ-(CH₂ )₀₋₃ heterocyclyl, —C(O)NR₃ₐ-(CH₂ )₀₋₃ heteroaryl, —C(O)NR₃ₐ-(CH₂ )₀₋₃ aryl;
wherein R₃ a is hydrogen or (1-2 C)alkyl;
wherein any (1-8 C)alkyl, (2-8 C)alkenyl, (2-8 C)alkynyl, (3-7 C)cycloalkyl, aryl, heteroaryl and heterocyclyl moiety is optionally substituted by one or more R₂₀₀ substituents;
wherein R₂₀₀ is selected from halo, (1-2 C)haloalkyl (e.g. trifluoromethyl), (1 -2 C)haloalkoxy (e.g. trifluoromethoxy), cyano, hydroxyl, nitro, (1-4 C)alkyl, (1-4 C)hydroxyalkyl, (CH₂ )_{z}ORₘ, (CH₂ )_{z} C(O)Rₘ, (CH₂ )_{z} C(O)ORₘ, (CH₂ )_{z}OC(O)Rₘ, (CH₂ )_{z} C(O)N(Rₒ )Rₚ, (CH₂ )_{z} N(Rₙ )C(O)Rₘ, (CH₂ )_{z} N(Rₙ )C(O)ORₘ, (CH₂ )_{z} S(O)_{y3} Rₘ, (CH₂ )_{z} SO₂ N(Rₒ )Rₚ, (CH₂ )_{z} N(Rₙ )SO₂ Rₘ, (CH₂ )_{z} NRₒ Rₚ, (CH₂ )_{z} (3-7 C)cycloalkyl, (CH₂ )_{z} heterocyclyl, (CH₂ )_{z} heteroaryl, (CH₂ )_{z} aryl;
and wherein:
(i) Rₘ and Rₙ are each independently selected from hydrogen, (1-6 C)alkyl or (CH₂ )₀₋₃ phenyl; Rₒ and Rₚ are each independently selected from hydrogen, (1-6 C)alkyl or phenyl or Rₒ and Rₚ together with the nitrogen atom to which they are attached form a 3-7 membered ring which may optionally include further heteroatoms, and wherein any 3-7 membered ring formed Rₒ and Rₚ, and any alkyl or phenyl group present for Rₘ, Rₙ, Rₒ and Rₚ is optionally further substituted by one or more substituents selected from halo, trifluoromethyl, trifluoromethoxy, cyano, hydroxyl, carboxyl, carbamoyl, sulphamoyl, and (1 -2 C)alkyl; and
(ii) any (3-7 C)cycloalkyl, heterocyclyl, heteroaryl or aryl moiety in a R₂₀₀ substituent group is optionally further substituted by one or more substituents selected from halo, trifluoromethyl, trifluoromethoxy, cyano, hydroxyl, (1-2 C)alkyl, (1 -2 C)haloalkyl, (1-2 C)hydroxyalkyl, OR_{q}, C(O)R_{q}, C(O)OR_{q}, OC(O)R_{q}, C(O)N(R_{q} )Rᵣ, N(Rᵣ )C(O)R_{q}, S(O)_{y4} R_{q}, SO₂ N(Rᵣ )R_{q}, N(Rᵣ )SO₂ R_{q}, or NRᵣ R_{q},
wherein R_{q} is hydrogen, (1-2 C)alkyl or phenyl, and R, are selected from hydrogen or (1-2 C)alkyl;
or R₂ and R₃ are linked such that together they form a -X₂ =CQ- group;
X₂ is selected from N and CRₐ ; wherein Rₐ is selected from hydrogen, fluoro, chloro, methyl, cyano, difluoromethyl, and trifluoromethyl; and
Q is hydrogen, halo, cyano or a group of the formula: 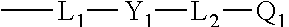
wherein:
L₁ is absent or (1-4 C)alkylene;
Y₁ is absent or O, S, SO, SO₂, N(Ry,), C(O), C(O)O, OC(O), C(O)N(R_{y1} ), or
N(R_{y1} )C(O), wherein R_{y1} is selected from hydrogen or (1-6 C)alkyl;
L₂ is absent or (1-3 C)alkylene; and
Q₁ is hydrogen, (1-6 C)alkyl, (2-6 C)alkenyl, (2-6 C)alkynyl, aryl, (3-8 C)cycloalkyl, heteroaryl or heterocyclyl;
wherein Q is optionally further substituted by one or more substituent groups independently selected from oxo, (1-6 C)alkyl, halo, (1-4 C)haloalkyl, (1 -4 C)haloalkoxy, (1-4 C)aminoalkyl, (1-4 C)hydroxyalkyl, cyano, or by one or more group(s) of the formula: 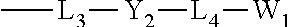
wherein:
L₃ is absent or (1-4 C)alkylene;
Y₂ is absent or selected from or O, S, SO, SO₂, N(R_{y2} ), C(O), C(O)O, OC(O), C(O)N(R_{y2} ), N(R_{y2} )C(O) or S(O)₂ N(R_{y2} ), N(R_{y2} )SO₂ wherein R_{y2} is selected from hydrogen or (1-4 C)alkyl;
L₄ is absent or (1-3 C)alkylene; and
W₁ is hydrogen, (1-6 C)alkyl, aryl, (3-8 C)cycloalkyl, heteroaryl or heterocyclyl;
wherein W₁ is optionally substituted by one or more substituents selected from oxo, (1-4 C)alkyl, halo, (1-4 C)haloalkyl, (1-4 C)haloalkoxy, (1 -4 C)alkoxy, amino, (1-4 C)alkylamino, di[(1-4 C)alkyl]amino C(O)OH, C(O)O(1-4 C)alkyl, (CH₂ )0 -₃ -heterocyclyl or cyano;
R₄ is selected from hydrogen or halo;
X₁ is N or CR₅, wherein Rₛ is selected from hydrogen, halo, cyano, or amino;
x is independently selected from 0, 1, 2 or 3;
y1, y2, y3 and y4 are independently selected from 0, 1 or 2;
z is independently selected from 0, 1, 2 or 3;
with the proviso that:
X₁ and X₂ are only N when Q is hydrogen;
when X₁ and X₂ are CR₅ or CRₐ, Q is not hydrogen;
Q₁ is not hydrogen when L₁, Y₁, and L₂ are all absent;
and at least one of R₁, Q, Rₐ, R₄ or R₅ is a substituent other than hydrogen.

2. A compound according to claim 1, or a pharmaceutically acceptable salt or solvate thereof, wherein the compound is a compound having the structural formula (Ia), (Ib) or (Ic) shown below: 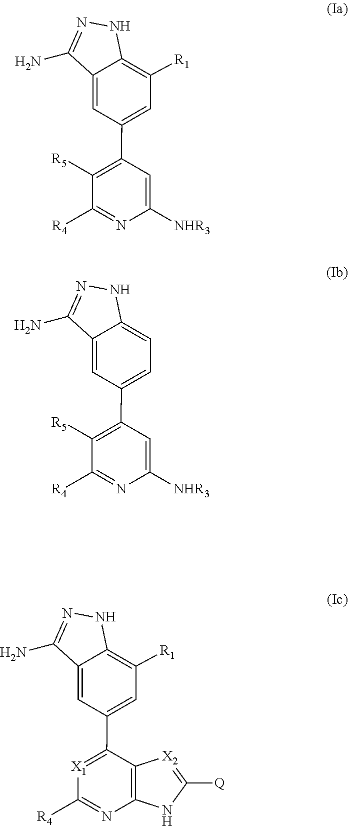
wherein R₁, X₁, X₂, R₃, R₄ and R₅ are each as defined in claim 1.

3. A compound according to claim 1 or claim 2, or a pharmaceutically acceptable salt or solvate thereof, wherein R₁ is selected from hydrogen, halogen, (2-6 C)alkynyl, (3-7 C)cycloalkyl, phenyl, a 5 or 6 -membered heteroaryl or a 4 to 7 -membered heterocyclyl,
wherein said (2-6 C)alkynyl, (3-7 C)cycloalkyl, phenyl, heteroaryl and heterocyclyl are optionally substituted by one or more R₁₀₀ substituents;
and wherein R₁₀₀ is selected from halo, (1-2 C)haloalkyl (e.g. trifluoromethyl) (1 -2 C)haloalkoxy e.g. trifluoromethoxy, cyano, hydroxyl, (1-4 C)alkyl, (1-4 C)hydroxyalkyl, (CH₂ )ₓOR_{f}, (CH₂ )ₓ C(O)R_{f}, (CH₂ )ₓ C(O)OR_{f}, (CH₂ )ₓOC(O)R_{f}, (CH₂ )ₓ C(O)N(Rⱼ )Rₕ, (CH₂ )ₓ N(R_{g} )C(O)R_{f}, (CH₂ )ₓ S(O)_{y1} R_{f}, (CH₂ )ₓ SO₂ N(Rⱼ )Rₕ, (CH₂ )ₓ N(R_{g} )SO₂ R_{f}, (CH₂ )ₓ NRⱼ Rₕ, (CH₂ )ₓ (3-7 C)cycloalkyl, (CH₂ )ₓ heterocyclyl, (CH₂ )ₓ heteroaryl (CH₂ )ₓ aryl;
and wherein:
(i) x and y1 are as defined in claim 1;
(ii) R_{f} and R_{g} are each independently selected from hydrogen or (1-2 C)alkyl; and wherein Rₕ and Rⱼ are each independently selected from hydrogen or (1 -2 C)alkyl or Rₕ and Rⱼ together with the nitrogen atom to which they are attached form a 3-7 membered ring which may optionally include further heteroatoms; and
(iii) any (1-4 C)alkyl, (3-7 C)cycloalkyl, heterocyclyl, heteroaryl or aryl moiety in a R₁₀₀ substituent group is optionally further substituted by one or more substituents selected from halo, trifluoromethyl, trifluoromethoxy, cyano, hydroxyl, (1 -2 C)alkyl, (1-2 C)haloalkyl, (1-2 C)hydroxyalkyl, ORₖ, C(O)Rₖ, C(O)ORₖ, OC(O)Rₖ, C(O)N(Rₗ )Rₖ, N(Rₗ )C(O)Rₖ, S(O)_{y2} Rₖ, SO₂ N(Rl)Rₖ, N(Rₗ )SO₂ Rₖ, or NRₗ Rₖ, wherein Rₖ and Rₗ are selected from hydrogen or (1-2 C)alkyl and y2 is independently selected from 0, 1 or 2.

4. A compound according to any one of the preceding claims, or a pharmaceutically acceptable salt or solvate thereof, wherein R₁ is selected from hydrogen, halogen, (2 -6 C)alkynyl, phenyl or a 5 or 6 -membered heteroaryl,
wherein said (2-6 C)alkynyl, phenyl or heteroaryl are optionally substituted by one or more R₁₀₀ substituents;
and wherein R₁₀₀ is selected from halo, (1-2 C)haloalkyl (e.g. trifluoromethyl), (1 -2 C)haloalkoxy e.g. (trifluoromethoxy), cyano, hydroxyl, (1-4 C)alkyl, (1-4 C)hydroxyalkyl, (CH₂ )ₓOR_{f}, (CH₂ )ₓ C(O)R_{f}, (CH₂ )ₓ C(O)OR_{f}, (CH₂ )ₓOC(O)R_{f}, (CH₂ )ₓ C(O)N(Rⱼ )Rₕ, (CH₂ )ₓ N(R_{g} )C(O)R_{f}, (CH₂ )ₓ S(O)_{y1} R_{f}, (CH₂ )ₓ SO₂ N(Rⱼ )Rₕ, (CH₂ )ₓ N(R₉ )SO₂ R_{f}, (CH₂ )ₓ NRⱼ Rₕ, (CH₂ )ₓ (3-7 C)cycloalkyl, (CH₂ )ₓ -[4-6 membered heterocyclyl], (CH₂ )ₓ -[5 or 6 membered heteroaryl]or (CH₂ )ₓ phenyl;
and wherein:
(i) x and y1 are as defined in claim 1;
(ii) R_{f} and R_{g} are each independently selected from hydrogen or (1-2 C)alkyl; and
wherein Rₕ and Rⱼ are each independently selected from hydrogen or (1 -2 C)alkyl or Rₕ and Rⱼ together with the nitrogen atom to which they are attached form a 3-7 membered ring which may optionally include further heteroatoms; and
(iii) any (1-4 C)alkyl, (3-7 C)cycloalkyl, heterocyclyl, heteroaryl or aryl moiety in a R₁₀₀ substituent group is optionally further substituted by one or more substituents selected from halo, trifluoromethyl, trifluoromethoxy, cyano, hydroxyl, (1 -2 C)alkyl, (1-2 C)haloalkyl, (1-2 C)hydroxyalkyl ORₖ, C(O)Rₖ, C(O)ORₖ, OC(O)Rₖ, C(O)N(Rₗ )Rₖ, N(Rₗ )C(O)Rₖ, S(O)_{y2} Rₖ, SO₂ N(Rₗ )Rₖ, N(Rₗ )SO₂ Rₖ, or NRₗ Rₖ, wherein Rₖ and Rₗ are selected from hydrogen or (1-2 C)alkyl and y2 is independently selected from 0, 1 or 2.

5. A compound according to any one of the preceding claims, or a pharmaceutically acceptable salt or solvate thereof, wherein Rₗ is selected from hydrogen, halogen, (2 -6 C)alkynyl, phenyl or a 5 or 6 -membered heteroaryl,
wherein said (2-6 C)alkynyl, phenyl or heteroaryl are optionally substituted by one or more R₁₀₀ substituents;
and wherein R₁₀ o is selected from halo, trifluoromethyl, trifluoromethoxy, cyano, hydroxyl, (1-4 C)alkyl, (1-4 C)hydroxyalkyl, (CH₂ )ₓOR_{f}, (CH₂ )ₓ C(O)R_{f}, (CH₂ )ₓ C(O)OR_{f}, (CH₂ )ₓOC(O)Re, (CH₂ )ₓ C(O)N(Rⱼ )Rₕ, (CH₂ )ₓ N(R₉ )C(O)R_{f}, (CH₂ )ₓ S(O)y1 R_{f}, (CH₂ )ₓ SO₂ N(Rⱼ )Rₙ, (CH₂ )ₓ N(R_{g} )SO₂ R_{f}, (CH₂ )ₓ NRⱼ Rₕ, (CH₂ )ₓ (3-7 C)cycloalkyl, (CH₂ )ₓ -[4-6 membered heterocyclyl], (CH₂ )ₓ -[5 or 6 membered heteroaryl]or (CH₂ )ₓ phenyl;
and wherein:
(i) x and y1 are as defined in claim 1;
(ii) R_{f} and R_{g} are each independently selected from hydrogen or (1-2 C)alkyl; and
wherein Rₕ and Rⱼ are each independently selected from hydrogen or (1 -2 C)alkyl; and
(iii) any (1-4 C)alkyl, (3-7 C)cycloalkyl, heterocyclyl, heteroaryl or aryl moiety in a R₁₀₀ substituent group is optionally further substituted by one or more substituents selected from halo, trifluoromethyl, trifluoromethoxy, cyano, hydroxyl, (1 -2 C)alkyl, (1-2 C)haloalkyl, (1-2 C)hydroxyalkyl or ORₖ, wherein Rₖ is selected from hydrogen or (1-2 C)alkyl.

6. A compound according to any one of the preceding claims, or a pharmaceutically acceptable salt or solvate thereof, wherein R₁ is selected from hydrogen, halogen, (2 -6 C)alkynyl, phenyl or a 5 or 6 -membered heteroaryl,
wherein said (2-6 C)alkynyl, phenyl or heteroaryl are optionally substituted by one or more R₁₀₀ substituents;
and wherein R₁₀₀ is selected from halo, trifluoromethyl, trifluoromethoxy, cyano, hydroxyl, (1-4 C)alkyl, (1-4 C)hydroxyalkyl, (CH₂ )ₓOR_{f}, C(O)R_{f}, C(O)OR_{f}, OC(O)R_{f}, C(O)N(Rⱼ )Rₕ, N(R_{g} )C(O)R_{f}, S(O)_{y1} R_{f}, SO₂ N(Rⱼ )Rₕ, N(R_{g} )SO₂ R_{f}, NRⱼ Rₕ, (CH₂ )ₓ -[4-6 membered heterocyclyl], or (CH₂ )ₓ phenyl;
and wherein:
(i) x and y1 are as defined in claim 1;
(ii) R_{f} and R_{g} are each independently selected from hydrogen or (1-2 C)alkyl; and
wherein Rₕ and Rⱼ are each independently selected from hydrogen or (1 -2 C)alkyl; and
(iii) any (1-4 C)alkyl, (3-7 C)cycloalkyl, heterocyclyl, heteroaryl or aryl moiety in a R₁ io substituent group is optionally further substituted by one or more substituents selected from halo, trifluoromethyl, trifluoromethoxy, cyano, hydroxyl, (1 -2 C)alkyl, (1-2 C)haloalkyl, (1-2 C)hydroxyalkyl or ORₖ, wherein Rₖ is selected from hydrogen or (1-2 C)alkyl.

7. A compound according to any one of the preceding claims, or a pharmaceutically acceptable salt or solvate thereof, wherein R₁ is selected from:
(i) hydrogen or halogen;
(ii) ethynyl, i.e. 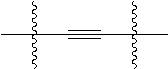 [SMILES: *C#C*]
which is optionally substituted by R₁₀₀ ;
(iv) phenyl, which is optionally substituted by R₁₀₀ ;
(v) a 5 or 6 -membered heteroaryl, which is optionally substituted by R₁₀₀ ;
and wherein R_{1f} o is selected from halo, trifluoromethyl, trifluoromethoxy, cyano, hydroxyl, (1-4 C)alkyl, (1-4 C)hydroxyalkyl, (CH₂ )ₓOR_{f}, C(O)R_{f}, C(O)OR_{f}, OC(O)R_{f}, C(O)N(R,)Rₕ, N(R_{g} )C(O)R_{f}, S(O)_{y1} R_{f}, SO₂ N(Rⱼ )Rₕ, N(R_{g} )SO₂ R_{f}, NRⱼ Rₕ, (CH₂ )ₓ -[4-6 membered heterocyclyl], or (CH₂ )ₓ phenyl;
and wherein:
(i) x is independently selected from 0, 1 or 2;
(ii) y1 is independently selected from 0, 1 or 2;
(iii) R_{f} and R_{g} are each independently selected from hydrogen or (1-2 C)alkyl; and wherein Rₕ and Rⱼ are each independently selected from hydrogen or (1-2 C)alkyl; and
(iv) any (1-4 C)alkyl, (3-7 C)cycloalkyl, heterocyclyl, heteroaryl or aryl moiety in a R₁₀₀ substituent group is optionally further substituted by one or more substituents selected from halo, trifluoromethyl, trifluoromethoxy, cyano, hydroxyl, (1-2 C)alkyl, (1-2 C)haloalkyl, (1-2 C)hydroxyalkyl or ORₖ, wherein Rₖ is selected from hydrogen or (1-2 C)alkyl.

8. A compound according to any one of the preceding claims, or a pharmaceutically acceptable salt or solvate thereof, wherein R₁ is selected from:
(i) hydrogen;
(ii) ethynyl, i.e. 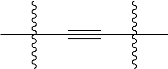 [SMILES: *C#C*]
which is optionally substituted by R₁₀₀ ;
(v) phenyl, which is optionally substituted by R₁₀₀ ;
(vi) a 5 or 6 -membered heteroaryl;
and wherein R₁₀₀ is selected from halo, trifluoromethyl, trifluoromethoxy, cyano, hydroxyl, (1-4 C)alkyl, (1-4 C)hydroxyalkyl, (CH₂ )ₓOR_{f}, C(O)R_{f}, C(O)OR_{f}, OC(O)R_{f}, C(O)N(Rⱼ )Rₕ, N(R_{g} )C(O)R_{f}, S(O)_{y1} Re, SO₂ N(Rⱼ )Rᵣ, N(R_{g} )SO₂ R_{f}, NRⱼ Rₕ, (CH₂ )ₓ -[4-6 membered heterocyclyl], or (CH₂ )ₓ phenyl;
and wherein:
(i) x is independently selected from 0, 1 or 2;
(ii) y1 is independently selected from 0, 1 or 2;
(iii) R_{f} and R_{g} are each independently selected from hydrogen or (1-2 C)alkyl; and wherein Rₕ and Rⱼ are each independently selected from hydrogen or (1 -2 C)alkyl; and
(iv) any (1-4 C)alkyl, (3-7 C)cycloalkyl, heterocyclyl, heteroaryl or aryl moiety in a R₁₀₀ substituent group is optionally further substituted by one or more substituents selected from halo, trifluoromethyl, trifluoromethoxy, cyano, hydroxyl, (1 -2 C)alkyl, (1-2 C)haloalkyl, (1-2 C)hydroxyalkyl or ORₖ, wherein Rₖ is selected from hydrogen or (1-2 C)alkyl.

9. A compound according to any one of the preceding claims, or a pharmaceutically acceptable salt or solvate thereof, wherein R₁ is selected from:
(i) hydrogen or halogen;
(ii) ethynyl, i.e. 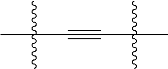 [SMILES: *C#C*]
which is optionally substituted by R₁₀₀ ;
(iii) phenyl, which is optionally substituted by R₁₀₀ ;
and wherein R₁₀₀ is selected from halo, trifluoromethyl, trifluoromethoxy, cyano, hydroxyl or (1-4 C)alkyl.

10. A compound according to any one of the preceding claims, or a pharmaceutically acceptable salt or solvate thereof, wherein R₃ is selected from hydrogen, (1-8 C)alkyl, (2 -8 C)alkenyl, (2-8 C)alkynyl, (3-7 C)cycloalkyl, phenyl, a 5 or 6 -membered heteroaryl, a 4 to 7 -membered heterocyclyl, (CH₂ )₀₋₃ (3-7 C)cycloalkyl, (CH₂ )₀₋₃[4 to 7 -membered heterocyclyl], (CH₂ )₀₋₃ [5 or 6 -membered heteroaryl], (CH₂ )₀₋₃phenyl, —C(O)-(CH₂ )₀₋₃ (3-7 C)cycloalkyl, —C(O)-(CH₂ )₀₋₃[4 to 7 -membered heterocyclyl], —C(O)-(CH₂ )₀₋₃[5 or 6 -membered heteroaryl], —C(O)-(CH₂ )₀₋₃phenyl, —C(O)O(1-8 C)alkyl, —C(O)NR₃ₐ-(1-8 C)alkyl, —C(O)NR₃ₐ-(CH₂ )₀₋₃ (3-7 C)cycloalkyl, —C(O)NR₃ₐ-(CH₂ )₀₋₃[5 to 7 -membered heterocyclyl], —C(O)NR₃ₐ-(CH₂ )₀ -₃[5 or 6 -membered heteroaryl], or —C(O)NR₃ₐ-(CH₂ )₀₋₃phenyl;
wherein R₃ₐ is hydrogen or (1-2 C)alkyl;
wherein any (1-8 C)alkyl, (2-8 C)alkenyl, (2-8 C)alkynyl, (3-7 C)cycloalkyl, phenyl, 5 or 6 -membered heteroaryl or 4 to 7 -membered heterocyclyl moiety is optionally substituted by one or more R₂₀₀ substituents;
wherein R₂₀₀ is selected from halo, (1-2 C)haloalkyl (e.g. trifluoromethyl), (1 -2 C)haloalkoxy (e.g. trifluoromethoxy), cyano, hydroxyl, nitro, (1-4 C)alkyl, (1-4 C)hydroxyalkyl, (CH₂ )_{z}ORₘ, (CH₂ )_{z} C(O)Rₘ, (CH₂ )_{z} C(O)ORₘ, (CH₂ )_{z} C(O)N(Rₒ )Rₚ, (CH₂ )_{z} N(Rₙ )C(O)Rₘ, (CH₂ )₇ N(Rₙ )C(O)ORₘ, (CH₂ )_{z} S(O)_{y3} Rₘ, (CH₂ )_{z} SO₂ N(Rₒ )Rₚ, (CH₂ )_{z} N(Rₙ )SO₂ Rₘ, (CH₂ )_{z} NRₒ Rₚ, (CH₂ )_{z} (3-7 C)cycloalkyl, (CH₂ )_{z} [4 to 7 -membered heterocyclyl], (CH₂ )_{z}[5 or 6 -membered heteroaryl, (CH₂ )_{z} phenyl;
and wherein:
(i) z and y3 are as defined in claim 1;
(ii) Rₘ and Rₙ are each independently selected from hydrogen, (1-6 C)alkyl or (CH₂ )o-₃ phenyl; Rₒ and Rₚ are each independently selected from hydrogen, (1-6 C)alkyl or phenyl or Rₒ and Rₚ together with the nitrogen atom to which they are attached form a 3-7 membered ring which may optionally include further heteroatoms, and wherein any 3-7 membered ring formed Rₒ and Rₚ, and any alkyl or phenyl group present for Rₘ, Rₙ, Rₐ and Rₚ is optionally further substituted by one or more substituents selected from halo, trifluoromethyl, trifluoromethoxy, cyano, hydroxyl, carboxyl, carbamoyl, sulphamoyl, and (1 -2 C)alkyl; and
(iii) any (3-7 C)cycloalkyl, heterocyclyl, heteroaryl or phenyl moiety in a R₂₀₀ substituent group is optionally further substituted by one or more substituents selected from halo, trifluoromethyl, trifluoromethoxy, cyano, hydroxyl, (1 -2 C)alkyl, (1-2 C)haloalkyl, (1-2 C)hydroxyalkyl, OR_{q}, C(O)R_{q}, C(O)OR_{q}, OC(O)R_{q}, C(O)N(R_{q} )Rᵣ, N(Rᵣ )C(O)R_{q}, S(O)_{y4} R_{q}, SO₂ N(Rᵣ )R_{q}, N(Rᵣ )SO₂ R,, or NRᵣ R_{q}, wherein R_{q} is hydrogen, (1-2 C)alkyl or phenyl, Rᵣ are selected from hydrogen or (1-2 C)alkyl; and y4 is independently selected from 0, 1 or 2.

11. A compound according to any one of the preceding claims, or a pharmaceutically acceptable salt or solvate thereof, wherein R₃ is selected from hydrogen, (1-8 C)alkyl, (2 -8 C)alkenyl, (2-8 C)alkynyl, (3-7 C)cycloalkyl, phenyl, a 5 or 6 -membered heteroaryl, a 4 to 7 -membered heterocyclyl, (CH₂ )₀₋₃ (3-7 C)cycloalkyl, (CH₂ )_{0-3[4} to 7 -membered heterocyclyl], (CH₂ )_{0-3[5} or 6 -membered heteroaryl], (CH₂ )₀₋₃phenyl, —C(O)-(CH₂ )₀₋₃ (3-7 C)cycloalkyl, —C(O)-(CH₂ )₀₋₃[4 to 7 -membered heterocyclyl], —C(O)-(CH₂ )₀₋₃[5 or 6 -membered heteroaryl], -C(O)-(CH₂ )₀₋₃phenyl, —C(O)O(1-8 C)alkyl, —C(O)NR₃ₐ-(1-8 C)alkyl, —C(O)NR₃ₐ-(CH₂ )₀₋₃ (3 -7 C)cycloalkyl, —C(O)NR₃ₐ-(CH₂ )_{0-3[5} to 7 -membered heterocyclyl], —C(O)NR₃ₐ-(CH₂ )₀ -₃[5 or 6 -membered heteroaryl], or —C(O)NH—(CH₂ )₀₋₃phenyl;
wherein R₃ₐ is hydrogen or (1-2 C)alkyl;
wherein any (1-8 C)alkyl, (2-8 C)alkenyl, (2-8 C)alkynyl, (3-7 C)cycloalkyl, phenyl, 5 or 6 -membered heteroaryl or 4 to 7 -membered heterocyclyl moiety is optionally substituted by one or more R₂₀₀ substituents;
wherein R₂₀₀ is selected from halo, trifluoromethyl, trifluoromethoxy, cyano, hydroxyl, nitro, (1-4 C)alkyl, (1-4 C)hydroxyalkyl, (CH₂ )_{z}ORₘ, (CH₂ )_{z} C(O)Rₘ, (CH₂ )_{z} C(O)ORₘ, (CH₂ ),C(O)N(Rₒ )Rₚ, (CH₂ )_{z} N(Rₙ )C(O)Rₘ, (CH₂ )_{z} N(Rₙ )C(O)ORₘ, (CH₂ )_{z} S(O)_{y3} Rₘ, (CH₂ )ZSO₂ N(Rₒ )Rₚ, (CH₂ )_{z} N(Rₙ )SO₂ Rₘ, (CH₂ )_{z} NRₒ Rₚ, (CH₂ )_{z} (3-7 C)cycloalkyl, (CH₂ )_{z} [4 to 7 -membered heterocyclyl], (CH₂ )Z[5 or 6 -membered heteroaryl, (CH₂ )_{z} phenyl;
and wherein:
(i) z and y3 are as defined in claim 1; and
(ii) Rₘ and Rₙ are each independently selected from hydrogen, (1-6 C)alkyl or (CH₂ )o-₃ phenyl; Rₒ and Rₚ are each independently selected from hydrogen, (1-6 C)alkyl or phenyl or Rₒ and Rₚ together with the nitrogen atom to which they are attached form a 3-7 membered ring which may optionally include further heteroatoms, and wherein any 3-7 membered ring formed Rₒ and Rₚ, and any alkyl or phenyl group present for Rₘ, Rₙ, Rₗ and Rₚ is optionally further substituted by one or more substituents selected from halo, trifluoromethyl, trifluoromethoxy, cyano, hydroxyl, carboxyl, carbamoyl, sulphamoyl, and (1 -2 C)alkyl.

12. A compound according to any one of the preceding claims, or a pharmaceutically acceptable salt or solvate thereof, wherein R₃ is selected from hydrogen, (1-8 C)alkyl, (2 -8 C)alkenyl, (2-8 C)alkynyl, (3-7 C)cycloalkyl, phenyl, a 5 or 6 -membered heteroaryl, a 4 to 7 -membered heterocyclyl, (CH₂ )₀₋₃ (3-7 C)cycloalkyl, (CH₂ )₀₋₃[4 to 7 -membered heterocyclyl], (CH₂ )₀₋₃[5 or 6 -membered heteroaryl], (CH₂ )₀₋3 phenyl, —C(O)-(CH₂ )₀₋₃ (3-7 C)cycloalkyl, —C(O)-(CH₂ )₀₋₃[4 to 7 -membered heterocyclyl], —C(O)-(CH₂ )₀₋₃[5 or 6 -membered heteroaryl], —C(O)-(CH₂ )₀₋3 phenyl, —C(O)O(1-8 C)alkyl, —C(O)NR₃ₐ-(1-8 C)alkyl, —C(O)NR₃ₐ-(CH₂ )₀₋3 (3-7 C)cycloalkyl, —C(O)NR₃ₐ-(CH₂ )₀₋₃[5 to 7 -membered heterocyclyl], —C(O)NR₃ₐ-(CH₂ )₀₋₃[5 or 6 -membered heteroaryl], or —C(O)NR₃ₐ-(CH₂ )₀₋₃phenyl;
wherein R₃ₐ is hydrogen or (1-2 C)alkyl;
wherein any (1-8 C)alkyl, (2-8 C)alkenyl, (2-8 C)alkynyl, (3-7 C)cycloalkyl, phenyl, 5 or 6 -membered heteroaryl or 4 to 7 -membered heterocyclyl moiety is optionally substituted by one or more R₂₀₀ substituents;
wherein R₂₀₀ is selected from halo, trifluoromethyl, trifluoromethoxy, cyano, hydroxyl, nitro, (1-4 C)alkyl, (1-4 C)hydroxyalkyl, (CH₂ )_{z}ORₘ, (CH₂ )_{z} C(O)Rₘ, (CH₂ )_{z} C(O)ORₘ, (CH₂ )₇ C(O)N(Rₒ )Rₚ, (CH₂ )_{z} N(Rₙ )C(O)Rₘ, (CH₂ )_{z} N(Rₙ )C(O)ORₘ, (CH₂ )_{z} S(O)_{y3} Rₘ, (CH₂ )_{z} SO₂ N(Rₒ )Rₚ, (CH₂ )ZN(Rₙ )SO₂ Rₘ, or (CH₂ )_{z} NRₒ Rₚ ;
and wherein:
Rₘ and Rₒ are each independently selected from hydrogen, (1-6 C)alkyl or phenyl; Rₒ and Rₚ are each independently selected from hydrogen, (1-6 C)alkyl or (CH₂ )₀₋₂ phenyl or Rₒ and Rₚ together with the nitrogen atom to which they are attached form a 3-7 membered ring which may optionally include further heteroatoms, and wherein any 3-7 membered ring formed Rₒ and Rₚ, and any alkyl or phenyl group present for Rₘ, Rₙ, Rₒ and Rₚ is optionally further substituted by one or more substituents selected from halo, trifluoromethyl, trifluoromethoxy, cyano, hydroxyl, carboxyl, carbamoyl, sulphamoyl, and (1-2 C)alkyl; and
y3 is as defined in claim 1.

13. A compound according to any one of the preceding claims, or a pharmaceutically acceptable salt or solvate thereof, wherein R₃ is hydrogen or is selected from; 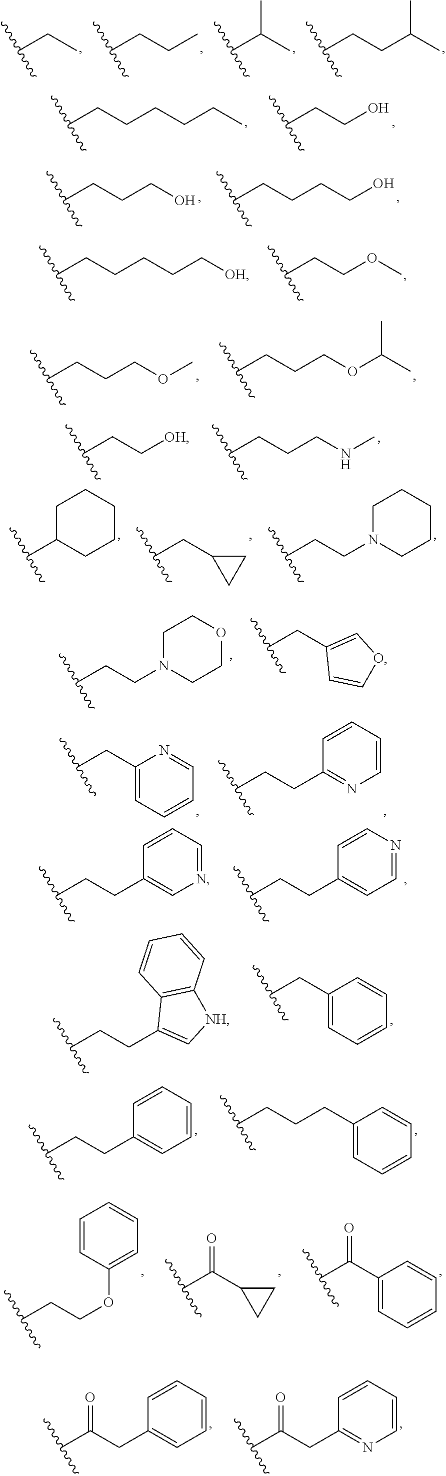 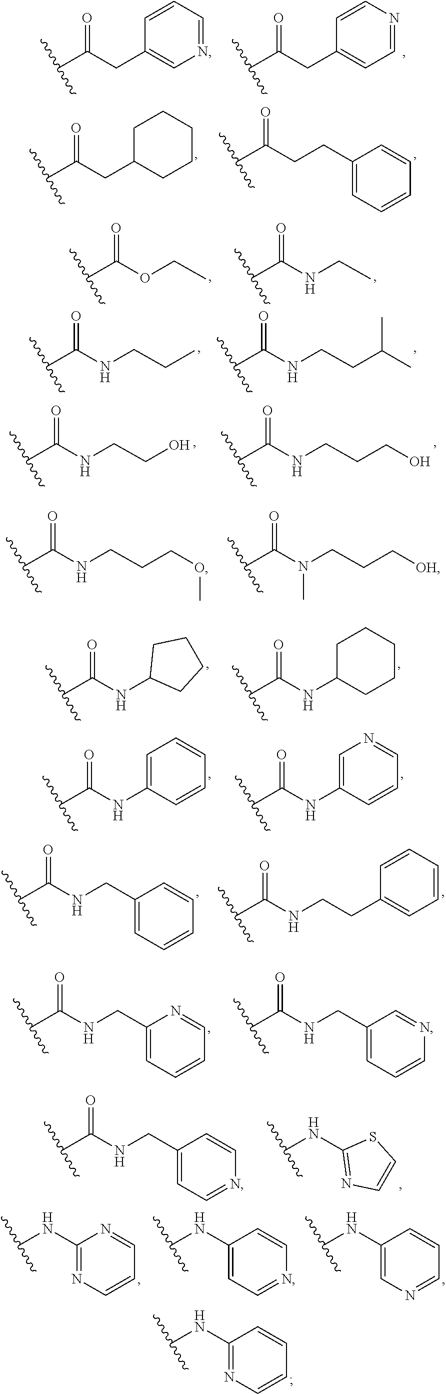
wherein any cycloalkyl, heterocyclyl, aryl or heteroaryl group above is optionally substituted by one or more R₂₀₀ substituents, wherein R₂₀₀ is as defined in any one of the preceding claims.

14. A compound according to any one of the preceding claims, or a pharmaceutically acceptable salt or solvate thereof, wherein the compound is compound of formula (Ia): 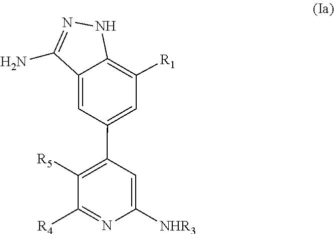
wherein R₁, R₃, R₄ and R₅ are each as defined in any one of claims 1 to 13.

15. A compound according to claim 14, or a pharmaceutically acceptable salt or solvate thereof, wherein R₃ is hydrogen and R₁ is a substituent other than hydrogen as defined in any one of claims 1 and 3 to 9.

16. A compound according to any one of claims 1 to 13, or a pharmaceutically acceptable salt or solvate thereof, wherein the compound is compound of formula (Ib): 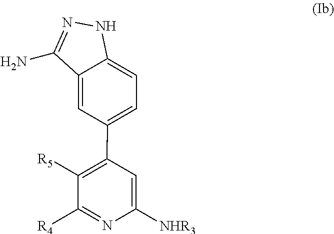
wherein R₃ is a substituent other than hydrogen as defined in in any one of claim 1 or 10 to 13 and R₄ and R₅ are each as defined in claim 1.

17. A compound according to any one of claims 1 to 13, or a pharmaceutically acceptable salt or solvate thereof, wherein the compound is compound of formula (Ic): 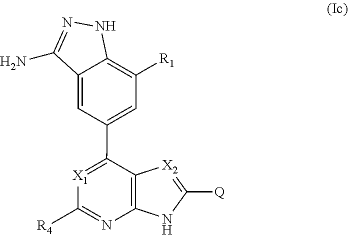
wherein R₁, R₄ X₁, X₂ and Q are each as defined in any one of claims 1 to 13.

18. A compound according to claim 17, or a pharmaceutically acceptable salt or solvate thereof, wherein X₂ is selected from N and CRₐ ; wherein Ra is selected from hydrogen, fluoro, chloro, methyl, or cyano.

19. A compound according to claim 17 or 18, or a pharmaceutically acceptable salt or solvate thereof, wherein Q is hydrogen, halo, cyano or a group of the formula: 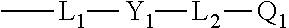
wherein:
L₁ is absent or (1-4 C)alkylene;
Y₁ is absent or O, S, SO, SO₂, N(R_{y1} ), C(O), C(O)O, OC(O), C(O)N(R_{y1} ), or N(R_{y1} )C(O), wherein R_{y1} is selected from hydrogen or (1-6 C)alkyl;
L₂ is absent or (1-3 C)alkylene; and
Q₁ is hydrogen, (1-6 C)alkyl, phenyl, (3-8 C)cycloalkyl, 5 or 6 -membered heteroaryl or 4 to 7 -membered heterocyclyl;
wherein Q is optionally further substituted by one or more substituent groups independently selected from oxo, (1-6 C)alkyl, halo, (1-4 C)haloalkyl, (1 -4 C)haloalkoxy, (1-4 C)aminoalkyl, (1-4 C)hydroxyalkyl, cyano, or by one or more group(s) of the formula: 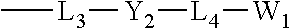
wherein:
L₃ is absent or (1-4 C)alkylene;
Y₂ is absent or selected from or O, S, SO, SO₂, N(R_{y2} ), C(O), C(O)O, OC(O), C(O)N(R_{y2} ), N(R_{y2} )C(O) or S(O)₂ N(R_{y2} ), N(R_{y2} )SO₂ wherein R_{y2} is selected from hydrogen or (1-4 C)alkyl;
L₄ is absent or (1-3 C)alkylene; and
W₁ is hydrogen, (1-6 C)alkyl, aryl, (3-8 C)cycloalkyl, 5 or 6 -membered heteroaryl or 4 to 7 -membered heterocyclyl;
wherein W₁ is optionally substituted by one or more substituents selected from oxo, (1-4 C)alkyl, halo, (1-4 C)haloalkyl, (1-4 C)haloalkoxy, (1 -4 C)alkoxy, amino, (1-4 C)alkylamino, di[(1-4 C)alkyl]amino C(O)OH, C(O)O(1-4 C)alkyl, (CH₂ )₀₋₃ -[4 to 7 -membered heterocyclyl]or cyano.
optionally wherein Q is a group of the formula: 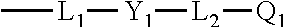
wherein:
L₁ is absent or (1-4 C)alkylene;
Y₁ is absent or O, S, SO, SO₂, N(R_{y1} ), C(O), C(O)O, OC(O), C(O)N(R_{y1} ), or N(Ry,)C(O), wherein R_{y1} is selected from hydrogen or (1-4 C)alkyl;
L₂ is absent or (1-3 C)alkylene; and
Q₁ is hydrogen, (1-6 C)alkyl, phenyl, (3-8 C)cycloalkyl, 5 or 6 -membered heteroaryl or 4 to 7 -membered heterocyclyl;
wherein Q is optionally further substituted by one or more substituent groups independently selected from oxo, (1-6 C)alkyl, halo, (1-4 C)haloalkyl, (1 -4 C)haloalkoxy, (1-4 C)aminoalkyl, (1-4 C)hydroxyalkyl, cyano, or by one or more group(s) of the formula: 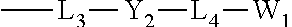
wherein:
L₃ is absent or (1-3 C)alkylene;
Y₂ is absent or selected from or O, S, SO, SO₂, N(R_{y2} ), C(O), C(O)O, OC(O), C(O)N(R_{y2} ), N(R_{y2} )C(O), S(O)₂ N(R_{y2} ) or N(R_{y2} )SO₂ wherein R_{y2} is selected from hydrogen or (1-2 C)alkyl;
L₄ is absent or (1-3 C)alkylene; and
W₁ is hydrogen, (1-6 C)alkyl, aryl, (3-8 C)cycloalkyl, 5 or 6 -membered heteroaryl or 4 to 7 -membered heterocyclyl;
wherein W₁ is optionally substituted by one or more substituents selected from oxo, (1-4 C)alkyl, halo, (1-4 C)haloalkyl, (1-4 C)haloalkoxy, (1 -4 C)alkoxy, amino, (1-4 C)alkylamino, di[(1-4 C)alkyl]amino C(O)OH, C(O)O(1-4 C)alkyl, (CH₂ )₀ -₃ -[4 to 7 -membered heterocyclyl]or cyano;
further optionally wherein:
L₁ is absent or (1-3 C)alkylene;
Y₁ is absent or O, S, N(R_{y1} ), C(O), C(O)O, C(O)N(R_{y1} ), or N(Ry,)C(O), wherein
R_{y1} is selected from hydrogen or (1-4 C)alkyl;
L₂ is absent or methylene; and
Q₁ is hydrogen, (1-6 C)alkyl, phenyl, (3-8 C)cycloalkyl, 5 or 6 -membered heteroaryl or 4 to 7 -membered heterocyclyl;
wherein Q is optionally further substituted by one or more substituent groups independently selected from oxo, (1-6 C)alkyl, halo, (1-2 C)haloalkyl, (1 -2 C)haloalkoxy, cyano, or by one or more group(s) of the formula: 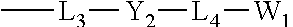
wherein:
L₃ is absent or (1-4 C)alkylene;
Y₂ is absent or selected from or 0, SO₂, N(R_{y2} ), C(O), C(O)O, C(O)N(R_{y2} ) or N(R_{y2} )SO₂ wherein R_{y2} is selected from hydrogen or (1-2 C)alkyl;
L₄ is absent or (1-3 C)alkylene; and
W₁ is hydrogen, (1-6 C)alkyl, aryl, (3-8 C)cycloalkyl, 5 or 6 -membered heteroaryl or 4 to 7 -membered heterocyclyl;
wherein W₁ is optionally substituted by one or more substituents selected from oxo, (1-4 C)alkyl, halo, (1-2 C)haloalkyl, (1-2 C)haloalkoxy, (1-4 C)alkoxy, C(O)OH, C(O)O(1-4 C)alkyl, (CH₂ )₀₋₃ -[4 to 7 -membered heterocyclyl]or cyano.

20. A compound according to any one of claims 17 to 19, or a pharmaceutically acceptable salt or solvate thereof, wherein Q is a group selected from hydrogen, halo, cyano, (1-6 C)alkyl, (1-6 C)alkoxy, (1-6 C)haloalkyl, (1-6 C)haloalkoxy, or a group of with a formula selected from: 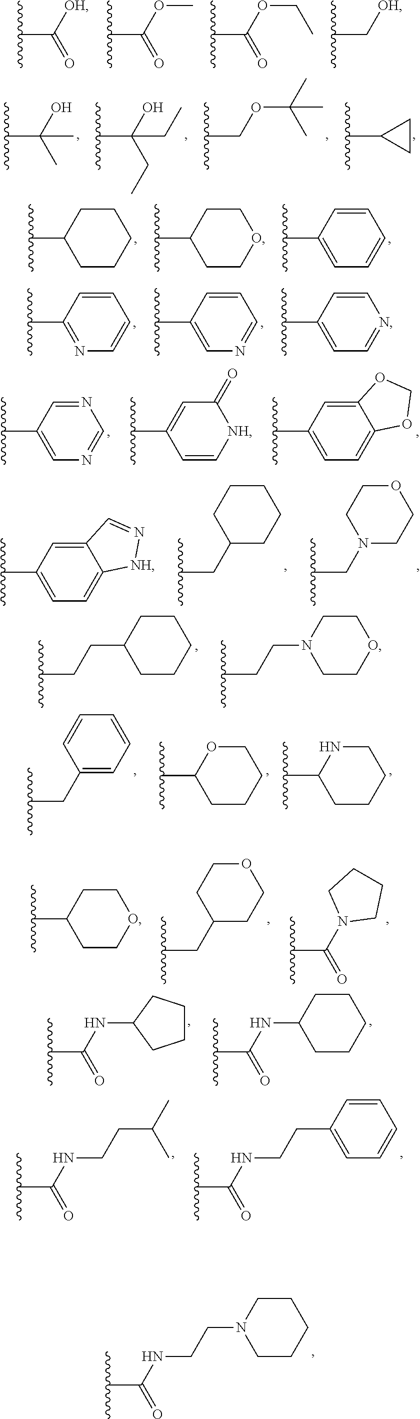 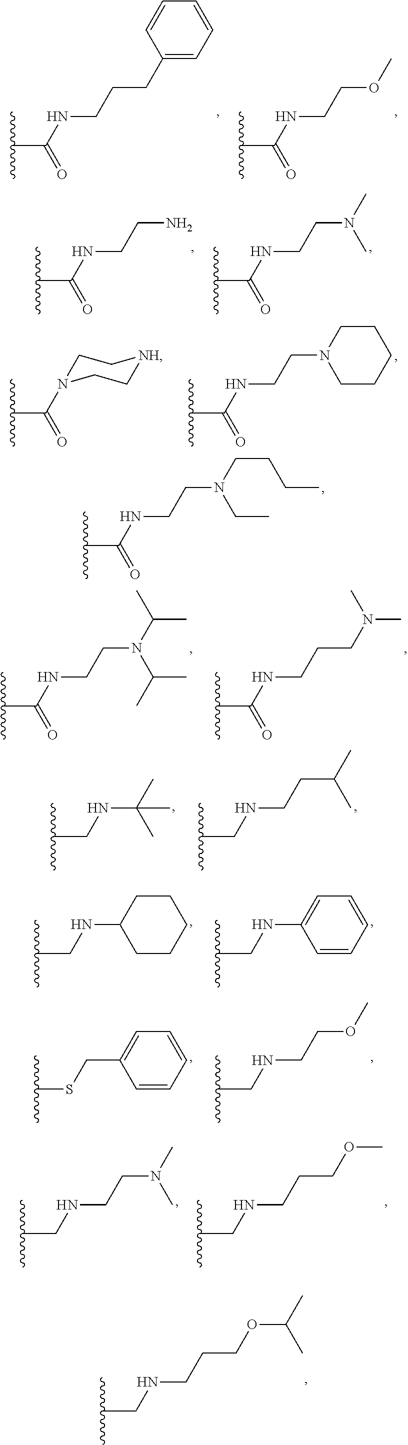 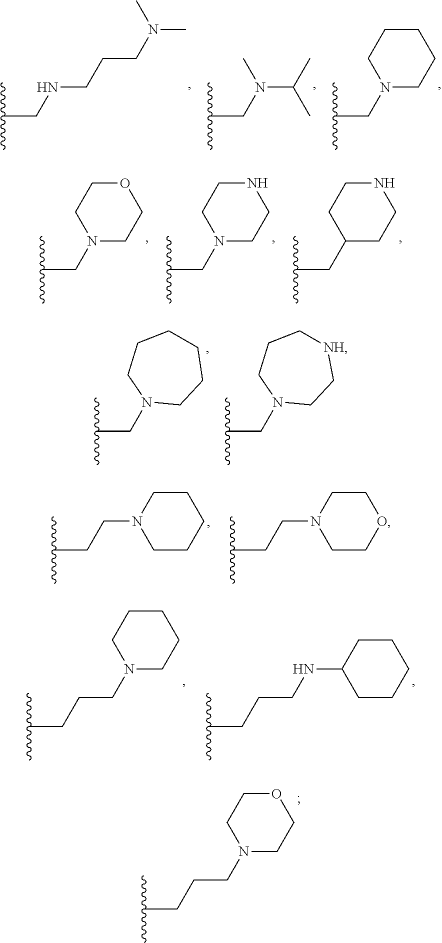
wherein any cycloalkyl, heterocyclyl, aryl or heteroaryl ring above may be optionally substituted by one or more substituents selected from oxo, (1-6 C)alkyl, halo, (1-2 C)haloalkyl, (1 -2 C)haloalkoxy, cyano, or by one or more group(s) of the formula:
-L₃ -Y₂ -L₄ -W ₁
wherein L₃, Y₂, L₄ and W₁ are as defined in any one of the preceding claims.

21. A compound according to any one of claims 17 to 20, or a pharmaceutically acceptable salt or solvate thereof, wherein R₄ is selected from hydrogen or fluoro.

22. A compound according to any one of claims 17 to 21, or a pharmaceutically acceptable salt or solvate thereof, wherein X₁ is N or CRS, wherein Rs is selected from hydrogen, halo, or cyano.

23. A compound according to any one of the preceding claims, or a pharmaceutically acceptable salt or solvate thereof, selected from:
5-(2 -aminopyridin-4 -yl)-7 -chloro-1 H-indazol-3 -amine;
5-(2 -aminopyridin-4 -yl)-7 -methyl-1 H-indazol-3 -amine;
5-(2 -aminopyridin-4 -yl)-7-(trifluoromethyl)-1 H-indazol-3 -amine;
5-(2-(ethylamino)pyridin-4 -yl)-1 H-indazol-3 -amine;
5-(2-(propylamino)pyridin-4 -yl)-1 H-indazol-3 -amine;
5-(2-(isopropylamino)pyridin-4 -yl)-1 H-indazol-3 -amine;
5-(2-((cyclopropylmethyl)amino)pyridin-4 -yl)-1 H-indazol-3 -amine;
5-(2-(isopentylamino)pyridin-4 -yl)-1 H-indazol-3 -amine;
5-(2-(hexylamino)pyridin-4 -yl)-1 H-indazol-3 -amine;
5-(2-(cyclohexylamino)pyridin-4 -yl)-1 H-indazol-3 -amine;
5 -{2 -[(Trans-4 -methylcyclohexyl)amino]pyridin-4 -yl}-1 H-indazol-3 -amine;
2-((4-(3 -amino-1 H-indazol-5 -yl)pyridin-2 -yl)amino)ethan-1 -ol;
3-((4-(3 -amino-1 H-indazol-5 -yl)pyridin-2 -yl)amino)propan-1 -ol;
4-((4-(3 -amino-1 H-indazol-5 -yl)pyridin-2 -yl)amino)butan-1 -ol;
5-((4-(3 -amino-1 H-indazol-5 -yl)pyridin-2 -yl)amino)pentan-1 -ol;
5 -{2 -[(trans-4 -hydroxycyclohexyl)amino]pyridin-4 -yl}-1 H-indazol-3 -amine;
5-(2-((2 -methoxyethyl)amino)pyridin-4 -yl)-1 H-indazol-3 -amine;
5-(2-((3 -methoxypropyl)amino)pyridin-4 -yl)-1 H-indazol-3 -amine;
5-(2-((3 -isopropoxypropyl)amino)pyridin-4 -yl)-1 H-indazol-3 -amine;
3-((4-(3 -amino-1 H-indazol-5 -yl)pyrimidin-2 -yl)amino)propan-1 -ol;
3-((4-(3 -amino-1 H-indazol-5 -yl)pyridin-2 -yl)(methyl)amino)propan-1 -ol;
5-(2-((2 -morpholinoethyl)amino)pyridin-4 -yl)-1 H-indazol-3 -amine;
5-(2-((2-(piperidin-1 -yl)ethyl)amino)pyridin-4 -yl)-1 H-indazol-3 -amine;
N¹-(4-(3 -amino-1 H-indazol-5 -yl)pyridin-2 -yl)-N₃ -methylpropane-1,3 -diamine;
5-(2-(benzylamino)pyridin-4 -yl)-1 H-indazol-3 -amine;
3-(((4-(3 -amino-1 H-indazol-5 -yl)pyridin-2 -yl)amino)methyl)benzonitrile;
5-(2-((3 -methoxybenzyl)amino)pyridin-4 -yl)-1 H-indazol-3 -amine;
2-(3-(((4-(3 -amino-1 H-indazol-5 -yl)pyridin-2 -yl)amino)methyl)phenyl)propan-2 -ol;
5-(2-((4-(trifluoromethyl)benzyl)amino)pyridin-4 -yl)-1 H-indazol-3 -amine;
4-(((4-(3 -amino-1 H-indazol-5 -yl)pyridin-2 -yl)amino)methyl)benzonitrile;
5-(2-((4-(tert-butyl)benzyl)amino)pyridin-4 -yl)-1 H-indazol-3 -amine;
2-(4-(((4-(3 -amino-1 H-indazol-5 -yl)pyridin-2 -yl)amino)methyl)phenyl)propan-2 -ol;
5-(2-((4-(methylsulfonyl)benzyl)amino)pyridin-4 -yl)-1 H-indazol-3 -amine;
5-(2-((furan-3 -ylmethyl)amino)pyridin-4 -yl)-1 H-indazol-3 -amine;
5-(2-((pyridin-2 -ylmethyl)amino)pyridin-4 -yl)-1 H-indazol-3 -amine;
5-(2-(phenethylamino)pyridin-4 -yl)-1 H-indazol-3 -amine;
5-(2-((2-(pyridin-2 -yl)ethyl)amino)pyridin-4 -yl)-1 H-indazol-3 -amine;
5-(2-((2-(pyridin-3 -yl)ethyl)amino)pyridin-4 -yl)-1 H-indazol-3 -amine;
5-(2-((2-(pyridin-4 -yl)ethyl)amino)pyridin-4 -yl)-1 H-indazol-3 -amine;
5-(2-((2-(1 H-indol-3 -yl)ethyl)amino)pyridin-4 -yl)-1 H-indazol-3 -amine;
5-(2-((4 -fluorophenethyl)amino)pyridin-4 -yl)-1 H-indazol-3 -amine;
5-(2-((4 -chlorophenethyl)amino)pyridin-4 -yl)-1 H-indazol-3 -amine;
4-(2-((4-(3 -amino-1 H-indazol-5 -yl)pyridin-2 -yl)amino)ethyl)phenol;
5-(2-((4 -methoxyphenethyl)amino)pyridin-4 -yl)-1 H-indazol-3 -amine;
5-(2-((4-(tert-butyl)phenethyl)amino)pyridin-4 -yl)-1 H-indazol-3 -amine;
4-(2-((4-(3 -amino-1 H-indazol-5 -yl)pyridin-2 -yl)amino)ethyl)benzenesulfonamide;
5-(2-((3 -chlorophenethyl)amino)pyridin-4 -yi)-1 H-indazol-3 -amine;
5-(2-((2-(trifluoromethyl)phenethyl)amino)pyridin-4 -yl)-1 H-indazol-3 -amine;
5-(2-((3 -phenylpropyl)amino)pyridin-4 -yl)-1 H-indazol-3 -amine;
5-(2-((2 -phenoxyethyl)amino)pyridin-4 -yl)-1 H-indazol-3 -amine;
N-(4-(3 -amino-1 H-indazol-5 -yl)pyridin-2 -yl)cyclopropanecarboxamide;
N-(4-(3 -amino-1 H-indazol-5 -yl)pyridin-2 -yl)benzamide;
N-(4-(3 -amino-1 H-indazol-5 -yl)pyridin-2 -yl)-2 -phenylacetamide;
N-(4-(3 -amino-1 H-indazol-5 -yl)pyridin-2 -yl)-2-(3 -fluorophenyl)acetamide;
N-(4-(3 -amino-1 H-indazol-5 -yl)pyridin-2 -yl)-2-(3 -tolyl)acetamide;
N-(4-(3 -amino-1 H-indazol-5 -yl)pyridin-2 -yl)-2-(3-(trifluoromethyl)phenyl)acetamide;
N-(4-(3 -amino-1 H-indazol-5 -yl)pyridin-2 -yl)-2-(3 -chlorophenyl)acetamide;
N-(4-(3 -amino-1 H-indazol-5 -yl)pyridin-2 -yl)-2-(3 -aminophenyl)acetamide;
N-(4-(3 -amino-1 H-indazol-5 -yl)pyridin-2 -yl)-2-(3 -nitrophenyl)acetamide;
N-(4-(3 -amino-1 H-indazol-5 -yl)pyridin-2 -yl)-2-(3-(methylsulfonyl)phenyl)acetamide;
N-(4-(3 -amino-1 H-indazol-5 -yl)pyridin-2 -yl)-2-(3 -methoxyphenyl)acetamide;
N-(4-(3 -amino-1 H-indazol-5 -yl)pyridin-2 -yl)-2-(3-(benzyloxy)phenyl)acetamide; tert-butyl (3-(2-((4-(3 -amino-1 H-indazol-5 -yl)pyridin-2 -yl)amino)-2 -oxoethyl)phenyl)carbamate;
N-(4-(3 -amino-1 H-indazol-5 -yl)pyridin-2 -yl)-2-(4 -fluorophenyl)acetamide;
N-(4-(3 -amino-1 H-indazol-5 -yl)pyridin-2 -yl)-2-(p-tolyl)acetamide;
N-(4-(3 -amino-1 H-indazol-5 -yl)pyridin-2 -yl)-2-(4-(trifluoromethyl)phenyl)acetamide;
N-(4-(3 -amino-1 H-indazol-5 -yl)pyridin-2 -yl)-2-(4 -chlorophenyl)acetamide;
N-(4-(3 -amino-1 H-indazol-5 -yl)pyridin-2 -yl)-2-(4-(methylthio)phenyl)acetamide;
N-(4-(3 -amino-1 H-indazol-5 -yl)pyridin-2 -yl)-2-(4-(methylsulfonyl)phenyl)acetamide;
N-(4-(3 -amino-1 H-indazol-5 -yl)pyridin-2 -yl)-2-(4 -methoxyphenyl)acetamide;
N-(4-(3 -amino-1 H-indazol-5 -yl)pyridin-2 -yl)-2-(4 -aminophenyl)acetamide;
tert-butyl (4-(2-((4-(3 -amino-1 H-indazol-5 -yl)pyridin-2 -yl)amino)-2 -oxoethyl)phenyl)carbamate;
N-(4-(3 -amino-1 H-indazol-5 -yl)pyridin-2 -yl)-2-(2 -fluorophenyl)acetamide;
N-(4-(3 -amino-1 H-indazol-5 -yl)pyridin-2 -yl)-2-(o-tolyl)acetamide;
N-(4-(3 -amino-1 H-indazol-5 -yl)pyridin-2 -yl)-2-(2-(trifluoromethyl)phenyl)acetamide;
N-(4-(3 -amino-1 H-indazol-5 -yl)pyridin-2 -yl)-2-(2 -chlorophenyl)acetamide;
N-(4-(3 -amino-1 H-indazol-5 -yl)pyridin-2 -yl)-2-(2 -methoxyphenyl)acetamide;
N-(4-(3 -amino-1 H-indazol-5 -yl)pyridin-2 -yl)-2-(pyridin-2 -yl)acetamide;
N-(4-(3 -amino-1 H-indazol-5 -yl)pyridin-2 -yl)-2-(pyridin-3 -yl)acetamide;
N-(4-(3 -amino-1 H-indazol-5 -yl)pyridine-2 -yl)-2-(pyridine-4 -yl)acetamide;
N-(4-(3 -amino-1 H-indazol-5 -yl)pyridine-2 -yl)-3 -phenylpropanamide;
ethyl (4-(3 -amino-1 H-indazol-5 -yl)pyridine-2 -yl)carbamate;
1-(4-(3 -amino-1 H-indazol-5 -yl)pyridine-2 -yl)-3 -ethylurea;
1-(4-(3 -amino-1 H-indazol-5 -yl)pyridine-2 -yl)-3 -propylurea;
1-(4-(3 -amino-1 H-indazol-5 -yl)pyridine-2 -yl)-3 -isopentylurea;
1-(4-(3 -amino-1 H-indazol-5 -yl)pyridine-2 -yl)-3 -cyclopentylurea;
1-(4-(3 -amino-1 H-indazol-5 -yl)pyridine-2 -yl)-3 -cyclohexylurea;
1-(4-(3 -amino-1 H-indazol-5 -yl)pyridine-2 -yl)-3-(2 -hydroxyethyl)urea;
1-(4-(3 -amino-1 H-indazol-5 -yl)pyridine-2 -yl)-3-(3 -hydroxypropyl)urea;
1-(4-(3 -amino-1 H-indazol-5 -yl)pyridine-2 -yl)-3-(2 -methoxyethyl)urea;
3-(4-(3 -amino-1 H-indazol-5 -yl)pyridine-2 -yl)-1-(2 -hydroxyethyl)-1 -methylurea;
1-(4-(3 -amino-1 H-indazol-5 -yl)pyridine-2 -yl)-3 -benzylurea;
1-(4-(3 -amino-1 H-indazol-5 -yl)pyridine-2 -yl)-3 -phenethylurea;
1-(4-(3 -amino-1 H-indazol-5 -yl)pyridine-2 -yl)-3-(pyridine-2 -ylmethyl)urea;
1-(4-(3 -amino-1 H-indazol-5 -yl)pyridine-2 -yl)-3-(pyridine-3 -ylmethyl)urea;
1-(4-(3 -amino-1 H-indazol-5 -yl)pyridine-2 -yl)-3-(pyridine-4 -ylmethyl)urea;
1-(4-(3 -amino-1 H-indazol-5 -yl)pyridine-2 -yl)-3 -phenylurea;
1-(4-(3 -amino-1 H-indazol-5 -yl)pyridine-2 -yl)-3-(3 -fluorophenyl)urea;
1-(4-(3 -amino-1 H-indazol-5 -yl)pyridine-2 -yl)-3-(3 -chlorophenyl)urea;
1-(4-(3 -amino-1 H-indazol-5 -yl)pyridine-2 -yl)-3-(3 -isopropylphenyl)urea;
1-(4-(3 -amino-1 H-indazol-5 -yl)pyridine-2 -yl)-3-(3-(hydroxymethyl)phenyl)urea;
3-(3-(4-(3 -amino-1 H-indazol-5 -yl)pyridine-2 -yl)ureido)benzamide;
1-(4-(3 -amino-1 H-indazol-5 -yl)pyridine-2 -yl)-3-(3 -phenoxyphenyl)urea;
1-(4-(3 -amino-1 H-indazol-5 -yl)pyridine-2 -yl)-3-(3-(benzyloxy)phenyl)urea;
1-(4-(3 -amino-1 H-indazol-5 -yl)pyridine-2 -yl)-3-(3-((4 -fluorobenzyl)oxy)phenyl)urea;
1-(4-(3 -amino-1 H-indazol-5 -yl)pyridine-2 -yl)-3-(3-((3 -fluorobenzyl)oxy)phenyl)urea;
1-(4-(3 -amino-1 H-indazol-5 -yl)pyridine-2 -yl)-3-(3-((2 -fluorobenzyl)oxy)phenyl)urea;
3-(3-(4-(3 -amino-1 H-indazol-5 -yl)pyridine-2 -yl)ureido)-N-phenylbenzamide;
1-(4-(3 -amino-1 H-indazol-5 -yl)pyridine-2 -yl)-3-(4 -fluorophenyl)urea;
1-(4-(3 -amino-1 H-indazol-5 -yl)pyridine-2 -yl)-3-(4 -chlorophenyl)urea;
1-(4-(3 -amino-1 H-indazol-5 -yl)pyridine-2 -yl)-3-(4-(tert-butyl)phenyl)urea;
1-(4-(3 -amino-1 H-indazol-5 -yl)pyridine-2 -yl)-3-(4-(methylsulfonyl)phenyl)urea;
1-(4-(3 -amino-1 H-indazol-5 -yl)pyridin-2 -yl)-3-(o-tolyl)urea;
1-(4-(3 -amino-1 H-indazol-5 -yl)pyridine-2 -yl)-3-(2 -ethylphenyl)urea;
1-(4-(3 -amino-1 H-indazol-5 -yl)pyridine-2 -yl)-3-(2 -isopropylphenyl)urea;
1-(4-(3 -amino-1 H-indazol-5 -yl)pyridine-2 -yl)-3-(pyridine-3 -yl)urea;
5-(2-(phenylamino)pyridine-4 -yl)-1 H-indazol-3 -amine;
5-(2-((3 -isopropylphenyl)amino)pyridine-4 -yl)-1 H-indazol-3 -amine;
3-((4-(3 -amino-1 H-indazol-5 -yl)pyridine-2 -yl)amino)phenol;
(3-((4-(3 -amino-1 H-indazol-5 -yl)pyridine-2 -yl)amino)phenyl)methanol;
3-((4-(3 -amino-1 H-indazol-5 -yl)pyridine-2 -yl)amino)benzoic acid;
ethyl 3-((4-(3 -amino-1 H-indazol-5 -yl)pyridine-2 -yl)amino)benzoate;
3-((4-(3 -amino-1 H-indazol-5 -yl)pyridine-2 -yl)amino)benzamide;
3-((4-(3 -amino-1 H-indazol-5 -yl)pyridine-2 -yl)amino)-N-(2 -hydroxyethyl)benzamide;
N1-(4-(3 -amino-1 H-indazol-5 -yl)pyridine-2 -yl)benzene-1,3 -diamine;
N-(3-((4-(3 -amino-1 H-indazol-5 -yl)pyridin-2 -yl)amino)phenyl)acetamide;
N-(3-((4-(3 -amino-1 H-indazol-5 -yl)pyridine-2 -yl)amino)phenyl)benzamide;
5-(2-((3 -phenoxyphenyl)amino)pyridine-4 -yl)-1 H-indazol-3 -amine;
5-(2-((3-(benzyloxy)phenyl)amino)pyridine-4 -yl)-1 H-indazol-3 -amine;
5-(2-((4 -fluorophenyl)amino)pyridine-4 -yl)-1 H-indazol-3 -amine;
5-(2-((4 -chlorophenyl)amino)pyridine-4 -yl)-1 H-indazol-3 -amine;
4-((4-(3 -amino-1 H-indazol-5 -yl)pyridine-2 -yl)amino)phenol;
5-(2-((4 -methoxyphenyl)amino)pyridine-4 -yl)-1 H-indazol-3 -amine;
5-(2-((4-(trifluoromethoxy)phenyl)amino)pyridine-4 -yl)-1 H-indazol-3 -amine;
5-(2-((4 -propoxyphenyl)amino)pyridine-4 -yl)-1 H-indazol-3 -amine;
2-((4-(3 -amino-1 H-indazol-5 -yl)pyridine-2 -yl)amino)phenol;
4-((4-(3 -amino-1 H-indazol-5 -yl)pyridine-2 -yl)amino)-2 -methylphenol;
5-((4-(3 -amino-1 H-indazol-5 -yl)pyridine-2 -yl)amino)-2 -methylphenol;
4-((4-(3 -amino-1 H-indazol-5 -yl)pyridine-2 -yl)amino)-3 -methylphenol;
5-(2-((3,4 -dichlorophenyl)amino)pyridine-4 -yl)-1 H-indazol-3 -amine;
N-(4-(3 -amino-1 H-indazol-5 -yl)pyridin-2 -yl)thiazol-2 -amine;
5-(2-(pyrimidin-2 -ylamino)pyridine-4 -yl)-1 H-indazol-3 -amine;
5-(2-(pyridine-4 -ylamino)pyridine-4 -yl)-1 H-indazol-3 -amine;
5-(2-(pyridine-3 -ylamino)pyridine-4 -yl)-1 H-indazol-3 -amine;
5-(2-(pyridine-2 -ylamino)pyridine-4 -yl)-1 H-indazol-3 -amine;
N2-(4-(3 -amino-1 H-indazol-5 -yl)pyridine-2 -yl)pyridine-2,6 -diamine;
N2-(4-(3 -amino-1 H-indazol-5 -yl)pyridine-2 -yl)-N6 -benzylpyridine-2,6 -diamine;
5-(7 H-pyrrolo[2,3 -b]pyrimidin-4 -yl)-1 H-indazol-3 -amine;
2 -amino-4-(3 -amino-1 H-indazol-5 -yl)-7 H-pyrrolo[2,3 -b]pyrimidine-5 -carbonitrile;
5-(3 H-imidazo[4,5 -b]pyridin-7 -yl)-1 H-indazol-3 -amine;
5-(3 -methyl-1 H-pyrrolo[2,3 -b]pyridin-4 -yl)-1 H-indazol-3 -amine;
5-(3 -chloro-1 H-pyrrolo[2,3 -b]pyridin-4 -yl)-1 H-indazol-3 -amine;
4-(3 -amino-1 H-indazol-5 -yl)-1 H-pyrrolo[2,3 -b]pyridine-3 -carbonitrile;
5-(2 -methyl-1 H-pyrrolo[2,3 -b]pyridin-4 -yl)-1 H-indazol-3 -amine;
5-(2-(tert-butyl)-1 H-pyrrolo[2,3 -b]pyridin-4 -yl)-1 H-indazol-3 -amine;
5-(2 -phenyl-1 H-pyrrolo[2,3 -b]pyridin-4 -yl)-1 H-indazol-3 -amine;
4-(3 -amino-1 H-indazol-5 -yl)-1 H-pyrrolo[2,3 -b]pyridine-2 -carboxylic acid;
5-(5 -fluoro-1 H-pyrrolo[2,3 -b]pyridin-4 -yl)-1 H-indazol-3 -amine;
5-(5 -chloro-1 H-pyrrolo[2,3 -b]pyridin-4 -yl)-1 H-indazol-3 -amine;
4-(3 -amino-1 H-indazol-5 -yl)-1 H-pyrrolo[2,3 -b]pyridine-5 -carbonitrile;
5-(7 H-pyrrolo[2,3 -b]pyrimidin-4 -yl)-1 H-indazol-3 -amine;
7 -bromo-5-(7 H-pyrrolo[2,3 -b]pyrimidin-4 -yl)-1 H-indazol-3 -amine;
5-(2 -methyl-1 H-pyrrolo[2,3 -b]pyridin-4 -yl)-1 H-indazol-3 -amine;
5-(2-(tert-butyl)-1 H-pyrrolo[2,3 -b]pyridin-4 -yl)-1 H-indazol-3 -amine;
5-(2 -cyclopropyl-1 H-pyrrolo[2,3 -b]pyridin-4 -yl)-1 H-indazol-3 -amine;
5-(2 -cyclohexyl-1 H-pyrrolo[2,3 -b]pyridin-4 -yl)-1 H-indazol-3 -amine;
5-(2 -neopentyl-1 H-pyrrolo[2,3 -b]pyridin-4 -yl)-1 H-indazol-3 -amine;
5-(2-(cyclohexylmethyl)-1 H-pyrrolo[2,3 -b]pyridin-4 -yl)-1 H-indazol-3 -amine;
5-(2-(2 -cyclohexylethyl)-1 H-pyrrolo[2,3 -b]pyridin-4 -yl)-1 H-indazol-3 -amine;
5-(2 -benzyl-1 H-pyrrolo[2,3 -b]pyridin-4 -yl)-1 H-indazol-3 -amine;
(4-(3 -amino-1 H-indazol-5 -yl)-1 H-pyrrolo[2,3 -b]pyridin-2 -yl)methanol;
2-(4-(3 -amino-1 H-indazol-5 -yl)-1 H-pyrrolo[2,3 -b]pyridin-2 -yl)propan-2 -ol;
3-(4-(3 -amino-1 H-indazol-5 -yl)-1 H-pyrrolo[2,3 -b]pyridin-2 -yl)pentan-3 -ol;
5-(2-(tert-butoxymethyl)-1 H-pyrrolo[2,3 -b]pyridin-4 -yl)-1 H-indazol-3 -amine;
5-(2-(tetrahydro-2 H-pyran-4 -yl)-1 H-pyrrolo[2,3 -b]pyridin-4 -yl)-1 H-indazol-3 -amine;
5-(2-(tetrahydro-2 H-pyran-2 -yl)-1 H-pyrrolo[2,3 -b]pyridin-4 -yl)-1 H-indazol-3 -amine;
5-(2-((tetrahydro-2 H-pyran-4 -yl)methyl)-1 H-pyrrolo[2,3 -b]pyridin-4 -yl)-1 H-indazol-3 -amine;
4-(3 -amino-1 H-indazol-5 -yl)-1 H-pyrrolo[2,3 -b]pyridine-2 -carboxylic acid;
methyl 4-(3 -amino-1 H-indazol-5 -yl)-1 H-pyrrolo[2,3 -b]pyridine-2 -carboxylate;
ethyl 4-(3 -amino-1 H-indazol-5 -yl)-1 H-pyrrolo[2,3 -b]pyridine-2 -carboxylate;
(4-(3 -amino-1 H-indazol-5 -yl)-1 H-pyrrolo[2,3 -b]pyridin-2 -yl)(pyrrolidin-1 -yl)methanone;
4-(3 -amino-1 H-indazol-5 -yl)-N-cyclopentyl-1 H-pyrrolo[2,3 -b]pyridine-2 -carboxamide;
4-(3 -amino-1 H-indazol-5 -yl)-N-cyclohexyl-1 H-pyrrolo[2,3 -b]pyridine-2 -carboxamide;
4-(3 -amino-1 H-indazol-5 -yl)-N-isopentyl-1 H-pyrrolo[2,3 -b]pyridine-2 -carboxamide;
4-(3 -amino-1 H-indazol-5 -yl)-N-phenethyl-1 H-pyrrolo[2,3 -b]pyridine-2 -carboxamide;
4-(3 -amino-1 H-indazol-5 -yl)-N-(3 -phenylpropyl)-1 H-pyrrolo[2,3 -b]pyridine-2 -carboxamide;
4-(3 -amino-1 H-indazol-5 -yl)-N-(2 -methoxyethyl)-1 H-pyrrolo[2,3 -b]pyridine-2 -carboxamide;
4-(3 -amino-1 H-indazol-5 -yl)-N-(2 -aminoethyl)-1 H-pyrrolo[2,3 -b]pyridine-2 -carboxamide;
4-(3 -amino-1 H-indazol-5 -yl)-N-(2-(dimethylamino)ethyl)-1 H-pyrrolo[2,3 -b]pyridine-2 -carboxamide;
(4-(3 -amino-1 H-indazol-5 -yl)-1 H-pyrrolo[2,3 -b]pyridin-2 -yl)(4 -methylpiperazin-1 -yl)methanone;
4-(3 -amino-1 H-indazol-5 -yl)-N-(2-(piperidin-1 -yl)ethyl)-1 H-pyrrolo[2,3 -b]pyridine-2 -carboxamide;
4-(3 -amino-1 H-indazol-5 -yl)-N-(2-(butyl(ethyl)amino)ethyl)-1 H-pyrrolo[2,3 -b]pyridine-2 -carboxamide;
4-(3 -amino-1 H-indazol-5 -yl)-N-(2-(diisopropylamino)ethyl)-1 H-pyrrolo[2,3 -b]pyridine-2 -carboxamide;
4-(3 -amino-1 H-indazol-5 -yl)-N-(3-(dimethylamino)propyl)-1 H-pyrrolo[2,3 -b]pyridine-2 -carboxamide;
5-(2-((tert-butylamino)methyl)-1 H-pyrrolo[2,3 -b]pyridine-4 -yl)-1 H-indazol-3 -amine;
5-(2-((isopentylamino)methyl)-1 H-pyrrolo[2,3 -b]pyridine-4 -yl)-1 H-indazol-3 -amine;
5-(2-(piperidin-2 -yl)-1 H-pyrrolo[2,3 -b]pyridine-4 -yl)-1 H-indazol-3 -amine;
5-(2-((cyclohexylamino)methyl)-1 H-pyrrolo[2,3 -b]pyridine-4 -yl)-1 H-indazol-3 -amine;
5-(2-((phenylamino)methyl)-1 H-pyrrolo[2,3 -b]pyridine-4 -yl)-1 H-indazol-3 -amine;
5-(2-(((2-(benzyloxy)phenyl)amino)methyl)-1 H-pyrrolo[2,3 -b]pyridine-4 -yl)-1 H-indazol-3 -amine;
5-(2-(((2 -methoxyethyl)amino)methyl)-1 H-pyrrolo[2,3 -b]pyridine-4 -yl)-1 H-indazol-3 -amine;
N1-((4-(3 -amino-1 H-indazol-5 -yl)-1 H-pyrrolo[2,3 -b]pyridin-2 -yl)methyl)-N2,N2 -dimethylethane-1,2 -diamine;
5-(2-(((3 -methoxypropyl)amino)methyl)-1 H-pyrrolo[2,3 -b]pyridine-4 -yl)-1 H-indazol-3 -amine;
5-(2-(((3 -isopropoxypropyl)amino)methyl)-1 H-pyrrolo[2,3 -b]pyridine-4 -yl)-1 H-indazol-3 -amine;
N1-((4-(3 -amino-1 H-indazol-5 -yl)-1 H-pyrrolo[2,3 -b]pyridin-2 -yl)methyl)-N3,N3 -dimethylpropane-1,3 -diamine;
5-(2-((isopropyl(methyl)amino)methyl)-1 H-pyrrolo[2,3 -b]pyridine-4 -yl)-1 H-indazol-3 -amine
5-(2-(piperidin-1 -ylmethyl)-1 H-pyrrolo[2,3 -b]pyridine-4 -yl)-1 H-indazol-3 -amine;
5-(2-((4,4 -difluoropiperidin-1 -yl)methyl)-1 H-pyrrolo[2,3 -b]pyridine-4 -yl)-1 H-indazol-3 -amine
5-(2-(morpholinomethyl)-1 H-pyrrolo[2,3 -b]pyridine-4 -yl)-1 H-indazol-3 -amine;
5-(2-((4 -methylpiperazin-1 -yl)methyl)-1 H-pyrrolo[2,3 -b]pyridine-4 -yl)-1 H-indazol-3 -amine;
5-(2-((4-(tert-butyl)piperazin-1 -yl)methyl)-1 H-pyrrolo[2,3 -b]pyridin-4 -yl)-1 H-indazol-3 -amine;
5-(2-(azepan-1 -ylmethyl)-1 H-pyrrolo[2,3 -b]pyridine-4 -yl)-1 H-indazol-3 -amine;
5-(2-((4 -methyl-1,4 -diazepan-1 -yl)methyl)-1 H-pyrrolo[2,3 -b]pyridin-4 -yl)-1 H-indazol-3 -amine;
5-(2-(2-(piperidin-1 -yl)ethyl)-1 H-pyrrolo[2,3 -b]pyridine-4 -yl)-1 H-indazol-3 -amine;
5-(2-(2 -morpholinoethyl)-1 H-pyrrolo[2,3 -b]pyridin-4 -yl)-1 H-indazol-3 -amine;
5-(2-(3-(piperidin-1 -yl)propyl)-1 H-pyrrolo[2,3 -b]pyridin-4 -yl)-1 H-indazol-3 -amine;
5-(2-(3-(cyclohexylamino)propyl)-1 H-pyrrolo[2,3 -b]pyridin-4 -yl)-1 H-indazol-3 -amine;
5-(2-(3 -morpholinopropyl)-1 H-pyrrolo[2,3 -b]pyridin-4 -yl)-1 H-indazol-3 -amine;
5-(2-(piperidin-4 -ylmethyl)-1 H-pyrrolo[2,3 -b]pyridin-4 -yl)-1 H-indazol-3 -amine;
5-(2-((1 -benzylpiperidin-4 -yl)methyl)-1 H-pyrrolo[2,3 -b]pyridin-4 -yl)-1 H-indazol-3 -amine
5-(2 -phenyl-1 H-pyrrolo[2,3 -b]pyridin-4 -yl)-1 H-indazol-3 -amine;
5-(2-(3-(trifluoromethyl)phenyl)-1 H-pyrrolo[2,3 -b]pyridin-4 -yl)-1 H-indazol-3 -amine;
3-(4-(3 -amino-1 H-indazol-5 -yl)-1 H-pyrrolo[2,3 -b]pyridin-2 -yl)benzonitrile;
3-(4-(3 -amino-1 H-indazol-5 -yl)-1 H-pyrrolo[2,3 -b]pyridin-2 -yl)phenol;
5-(2-(3 -methoxyphenyl)-1 H-pyrrolo[2,3 -b]pyridin-4 -yl)-1 H-indazol-3 -amine;
3-(4-(3 -amino-1 H-indazol-5 -yl)-1 H-pyrrolo[2,3 -b]pyridin-2 -yl)benzoic acid;
3-(4-(3 -amino-1 H-indazol-5 -yl)-1 H-pyrrolo[2,3 -b]pyridin-2 -yl)benzamide;
3-(4-(3 -amino-1 H-indazol-5 -yl)-1 H-pyrrolo[2,3 -b]pyridin-2 -yl)-N-(2 -methoxyethyl)benzamide;
3-(4-(3 -amino-1 H-indazol-5 -yl)-1 H-pyrrolo[2,3 -b]pyridin-2 -yl)-N-(2-(piperidin-1 -yl)ethyl)benzamide;
(3-(4-(3 -amino-1 H-indazol-5 -yl)-1 H-pyrrolo[2,3 -b]pyridin-2 -yl)phenyl)(4 -methylpiperazin-1 -yl)methanone;
5-(2-(3-(morpholinomethyl)phenyl)-1 H-pyrrolo[2,3 -b]pyridin-4 -yl)-1 H-indazol-3 -amine;
5-(2-(3-(methylsulfonyl)phenyl)-1 H-pyrrolo[2,3 -b]pyridin-4 -yl)-1 H-indazol-3 -amine;
5-(2-(3-(morpholinosulfonyl)phenyl)-1 H-pyrrolo[2,3 -b]pyridin-4 -yl)-1 H-indazol-3 -amine;
5-(2-(3 -aminophenyl)-1 H-pyrrolo[2,3 -b]pyridin-4 -yl)-1 H-indazol-3 -amine;
N-(3-(4-(3 -amino-1 H-indazol-5 -yl)-1 H-pyrrolo[2,3 -b]pyridin-2 -yl)phenyl)-3 -methoxypropanamide;
N-(3-(4-(3 -amino-1 H-indazol-5 -yl)-1 H-pyrrolo[2,3 -b]pyridin-2 -yl)phenyl)-3-(piperidin-1 -yl)propanamide;
4-((3-(4-(3 -amino-1 H-indazol-5 -yl)-1 H-pyrrolo[2,3 -b]pyridin-2 -yl)phenyl)amino)-4 -oxobutanoic acid;
N-(3-(4-(3 -amino-1 H-indazol-5 -yl)-1 H-pyrrolo[2,3 -b]pyridin-2 -yl)phenyl)methanesulfonamide
N-(3-(4-(3 -amino-1 H-indazol-5 -yl)-1 H-pyrrolo[2,3 -b]pyridin-2 -yl)phenyl)-4 -methylbenzenesulfonamide;
5-(2-(4-(morpholinomethyl)phenyl)-1 H-pyrrolo[2,3 -b]pyridin-4 -yl)-1 H-indazol-3 -amine
N-(4-(4-(3 -amino-1 H-indazol-5 -yl)-1 H-pyrrolo[2,3 -b]pyridin-2 -yl)phenyl)-4 -methylbenzenesulfonamide;
2-(4-(3 -amino-1 H-indazol-5 -yl)-1 H-pyrrolo[2,3 -b]pyridin-2 -yl)phenol;
5-(2-(2 -ethoxyphenyl)-1 H-pyrrolo[2,3 -b]pyridin-4 -yl)-1 H-indazol-3 -amine;
5-(2-(2 -ethylphenyl)-1 H-pyrrolo[2,3 -b]pyridin-4 -yl)-1 H-indazol-3 -amine;
5-(2-(benzo[d][1,3 ]dioxol-5 -yl)-1 H-pyrrolo[2,3 -b]pyridin-4 -yl)-1 H-indazol-3 -amine;
4-(4-(3 -amino-1 H-indazol-5 -yl)-1 H-pyrrolo[2,3 -b]pyridin-2 -yl)-2 -fluorobenzonitrile;
5-(4-(3 -amino-1 H-indazol-5 -yl)-1 H-pyrrolo[2,3 -b]pyridin-2 -yl)-2-(pyrrolidin-1 -yl)benzonitrile;
5-(4-(3 -amino-1 H-indazol-5 -yl)-1 H-pyrrolo[2,3 -b]pyridin-2 -yl)-2-(4 -methylpiperazin-1 -yl)benzonitrile;
5,5 ′-(1 H-pyrrolo[2,3 -b]pyridine-2,4 -diyl)bis(1 H-indazol-3 -amine);
5-(2-(3,5 -difluorophenyl)-1 H-pyrrolo[2,3 -b]pyridin-4 -yl)-1 H-indazol-3 -amine;
5-(2-(2,3,5 -trifluorophenyl)-1 H-pyrrolo[2,3 -b]pyridin-4 -yl)-1 H-indazol-3 -amine;
5-(2-(pyridin-2 -yl)-1 H-pyrrolo[2,3 -b]pyridin-4 -yl)-1 H-indazol-3 -amine;
5-(2-(pyridin-3 -yl)-1 H-pyrrolo[2,3 -b]pyridin-4 -yl)-1 H-indazol-3 -amine;
5-(2-(pyridin-4 -yl)-1 H-pyrrolo[2,3 -b]pyridin-4 -yl)-1 H-indazol-3 -amine;
5-(2-(pyrimidin-5 -yl)-1 H-pyrrolo[2,3 -b]pyridin-4 -yl)-1 H-indazol-3 -amine;
4-(4-(3 -amino-1 H-indazol-5 -yl)-1 H-pyrrolo[2,3 -b]pyridin-2 -yl)pyridin-2 (1 H)-one;
5-(2-(2 -fluoropyridin-4 -yl)-1 H-pyrrolo[2,3 -b]pyridin-4 -yl)-1 H-indazol-3 -amine;
5-(2-(2 -fluoro-6 -methylpyridin-4 -yl)-1 H-pyrrolo[2,3 -b]pyridin-4 -yl)-1 H-indazol-3 -amine;
5-(2-(2,6 -difluoropyridin-4 -yl)-1 H-pyrrolo[2,3 -b]pyridin-4 -yl)-1 H-indazol-3 -amine;
5-(2-(2-(piperidin-1 -yl)pyridin-4 -yl)-1 H-pyrrolo[2,3 -b]pyridin-4 -yl)-1 H-indazol-3 -amine;
5-(2-(2-(piperazin-1 -yl)pyridin-4 -yl)-1 H-pyrrolo[2,3 -b]pyridin-4 -yl)-1 H-indazol-3 -amine;
5-(2-(2 -morpholinopyridin-4 -yl)-1 H-pyrrolo[2,3 -b]pyridin-4 -yl)-1 H-indazol-3 -amine;
5-(2-(2-(4-(tert-butyl)piperazin-1 -yl)pyridin-4 -yl)-1 H-pyrrolo[2,3 -b]pyridin-4 -yl)-1 H-indazol-3 -amine;
4-(4-(4-(3 -amino-1 H-indazol-5 -yl)-1 H-pyrrolo[2,3 -b]pyridin-2 -yl)pyridin-2 -yl)thiomorpholine 1,1 -dioxide;
5-(2-(2,6 -dimorpholinopyridin-4 -yl)-1 H-pyrrolo[2,3 -b]pyridin-4 -yl)-1 H-indazol-3 -amine;
4-(4-(3 -amino-1 H-indazol-5 -yl)-1 H-pyrrolo[2,3 -b]pyridin-2 -yl)-1-(4-(piperidin-1 -ylmethyl)benzyl)pyridin-2 (1 H)-one;
4-(4-(3 -amino-1 H-indazol-5 -yl)-1 H-pyrrolo[2,3 -b]pyridin-2 -yl)-1-(3-(piperidin-1 -ylmethyl)benzyl)pyridin-2 (1 H)-one;
4-(4-(3 -amino-1 H-indazol-5 -yl)-1 H-pyrrolo[2,3 -b]pyridin-2 -yl)-1-(2-(piperidin-1 -ylmethyl)benzyl)pyridin-2 (1 H)-one;
5-(2-(5 -methoxypyridin-3 -yl)-1 H-pyrrolo[2,3 -b]pyridin-4 -yl)-1 H-indazol-3 -amine;
5-(2-(6 -morpholinopyridin-3 -yl)-1 H-pyrrolo[2,3 -b]pyridin-4 -yl)-1 H-indazol-3 -amine;
5-(2-(6-(4 -methylpiperazin-1 -yl)pyridin-3 -yl)-1 H-pyrrolo[2,3 -b]pyridin-4 -yl)-1 H-indazol-3 -amine;
5-(2-(6-((2 -morpholinoethyl)amino)pyridin-3 -y)-1 H-pyrrolo[2,3 -b]pyridin-4 -yl)-1 H-indazol-3 -amine;
5-(2-(2 -fluoropyridin-3 -yl)-1 H-pyrrolo[2,3 -b]pyridin-4 -yl)-1 H-indazol-3 -amine;
5-(2-(2 -morpholinopyridin-3 -yl)-1 H-pyrrolo[2,3 -b]pyridin-4 -yl)-1 H-indazol-3 -amine;
5-(2-(3 -methoxyphenyl)-1 H-pyrrolo[2,3 -b]pyridin-4 -yl)-1 H-indazol-3 -amine;
5-(2-(3 -isobutoxyphenyl)-1 H-pyrrolo[2,3 -b]pyridin-4 -yl)-1 H-indazol-3 -amine;
5-(2-(3-((tetrahydro-2 H-pyran-4 -yl)methoxy)phenyl)-1 H-pyrrolo[2,3 -b]pyridin-4 -yl)-1 H-indazol-3 -amine;
5-(2-(3-(2 -morpholinoethoxy)phenyl)-1 H-pyrrolo[2,3 -b]pyridin-4 -yl)-1 H-indazol-3 -amine;
5-(2-(3-(benzyloxy)phenyl)-1 H-pyrrolo[2,3 -b]pyridin-4 -yl)-1 H-indazol-3 -amine;
5-(2-(3-((3,5 -dimethoxybenzyl)oxy)phenyl)-1 H-pyrrolo[2,3 -b]pyridin-4 -yl)-1 H-indazol-3 -amine;
5-(2-(3-((4 -fluorobenzyl)oxy)phenyl)-1 H-pyrrolo[2,3 -b]pyridin-4 -yl)-1 H-indazol-3 -amine;
5-(2-(3-((2 -fluorobenzyl)oxy)phenyl)-1 H-pyrrolo[2,3 -b]pyridin-4 -yl)-1 H-indazol-3 -amine;
5-(2-(3-(pyridin-2 -ylmethoxy)phenyl)-1 H-pyrrolo[2,3 -b]pyridin-4 -yl)-1 H-indazol-3 -amine;
3-(4-(3 -amino-1 H-indazol-5 -yl)-1 H-pyrrolo[2,3 -b]pyridin-2 -yl)-5 -fluorophenol;
5-(2-(3 -fluoro-5 -methoxyphenyl)-1 H-pyrrolo[2,3 -b]pyridin-4 -yl)-1 H-indazol-3 -amine;
5-(2-(3 -fluoro-5-(2 -methoxyethoxy)phenyl)-1 H-pyrrolo[2,3 -b]pyridine-4 -yl)-1 H-indazol-3 -amine;
5-(2-(3 -fluoro-5-((tetrahydro-2 H-pyran-4 -yl)methoxy)phenyl)-1 H-pyrrolo[2,3 -b]pyridine-4 -yl)-1 H-indazol-3 -amine;
5-(2-(3-(benzyloxy)-5 -fluorophenyl)-1 H-pyrrolo[2,3 -b]pyridin-4 -yl)-1 H-indazol-3 -amine;
5-(2-(3-(benzyloxy)-5-(trifluoromethoxy)phenyl)-1 H-pyrrolo[2,3 -b]pyridin-4 -yl)-1 H-indazol-3 -amine;
methyl 3-(4-(3 -amino-1 H-indazol-5 -yl)-1 H-pyrrolo[2,3 -b]pyridin-2 -yl)-5-(benzyloxy)benzoate
5-(2-(3-(benzyloxy)-5-((2 -methoxyethoxy)methyl)phenyl)-1 H-pyrrolo[2,3 -b]pyridin-4 -yl)-1 H-indazol-3 -amine;
5-(2-(3-(benzyloxy)-5-((2 -methoxyethyl)amino)phenyl)-1 H-pyrrolo[2,3 -b]pyridin-4 -yl)-1 H-indazol-3 -amine;
5-(2-(3-(benzyloxy)-5-((2 -morpholinoethyl)amino)phenyl)-1 H-pyrrolo[2,3 -b]pyridin-4 -yl)-1 H-indazol-3 -amine;
5-(2-(2-(benzyloxy)phenyl)-1 H-pyrrolo[2,3 -b]pyridin-4 -yl)-1 H-indazol-3 -amine;
5-(2-(4-(benzyloxy)phenyl)-1 H-pyrrolo[2,3 -b]pyridin-4 -yl)-1 H-indazol-3 -amine;
5-(2-(5-(benzyloxy)pyridin-3 -yl)-1 H-pyrrolo[2,3 -b]pyridin-4 -yl)-1 H-indazol-3 -amine;
5-(2-(2-(benzyloxy)pyridin-4 -yl)-1 H-pyrrolo[2,3 -b]pyridin-4 -yl)-1 H-indazol-3 -amine;
5-(2-(6-(benzyloxy)pyridin-3 -yl)-1 H-pyrrolo[2,3 -b]pyridin-4 -yl)-1 H-indazol-3 -amine;
5-(2-(4-(benzyloxy)pyridin-2 -yl)-1 H-pyrrolo[2,3 -b]pyridin-4 -yl)-1 H-indazol-3 -amine;
5-(2-(2-(pyrimidin-5 -ylmethoxy)pyridin-4 -yl)-1 H-pyrrolo[2,3 -b]pyridin-4 -yl)-1 H-indazol-3 -amine;
5-(2-(2-(pyridin-4 -ylmethoxy)pyridin-4 -yl)-1 H-pyrrolo[2,3 -b]pyridin-4 -yl)-1 H-indazol-3 -amine;
5-(2-(2-(pyridin-3 -ylmethoxy)pyridin-4 -yl)-1 H-pyrrolo[2,3 -b]pyridin-4 -yl)-1 H-indazol-3 -amine;
5-(2-(2-(pyridin-2 -ylmethoxy)pyridin-4 -yl)-1 H-pyrrolo[2,3 -b]pyridin-4 -yl)-1 H-indazol-3 -amine;
5-(2-(6-(benzylamino)pyridin-3 -yl)-1 H-pyrrolo[2,3 -b]pyridin-4 -yl)-1 H-indazol-3 -amine;
5-(2-(2-(benzylamino)pyridin-4 -yl)-1 H-pyrrolo[2,3 -b]pyridin-4 -yl)-1 H-indazol-3 -amine;
5-(2-(2-(benzyl(methyl)amino)pyridin-4 -yl)-1 H-pyrrolo[2,3 -b]pyridin-4 -yl)-1 H-indazol-3 -amine;
5-(2-(2-(benzylthio)pyridin-4 -yl)-1 H-pyrrolo[2,3 -b]pyridin-4 -yl)-1 H-indazol-3 -amine;
5-(2-(2-(benzylthio)-6 -morpholinopyridin-4 -yl)-1 H-pyrrolo[2,3 -b]pyridin-4 -yl)-1 H-indazol-3 -amine;
5-(2-(3 -phenethylphenyl)-1 H-pyrrolo[2,3 -b]pyridin-4 -yl)-1 H-indazol-3 -amine;
5-(2-(2 -phenethoxypyridin-4 -yl)-1 H-pyrrolo[2,3 -b]pyridin-4 -yl)-1 H-indazol-3 -amine;
5-(2-(3-((phenylamino)methyl)phenyl)-1 H-pyrrolo[2,3 -b]pyridin-4 -yl)-1 H-indazol-3 -amine;
5-(2-(3-((tert-butylamino)methyl)phenyl)-1 H-pyrrolo[2,3 -b]pyridin-4 -yl)-1 H-indazol-3 -amine;
5-(2-(3-((cyclopentylamino)methyl)phenyl)-1 H-pyrrolo[2,3 -b]pyridin-4 -yl)-1 H-indazol-3 -amine;
5-(2-(3-((cyclohexylamino)methyl)phenyl)-1 H-pyrrolo[2,3 -b]pyridin-4 -yl)-1 H-indazol-3 -amine;
5-(2-(3-((butylamino)methyl)phenyl)-1 H-pyrrolo[2,3 -b]pyridin-4 -yl)-1 H-indazol-3 -amine;
5-(2-(3-((isopentylamino)methyl)phenyl)-1 H-pyrrolo[2,3 -b]pyridin-4 -yl)-1 H-indazol-3 -amine
5-(2-(3-((butyl(ethyl)amino)methyl)phenyl)-1 H-pyrrolo[2,3 -b]pyridin-4 -yl)-1 H-indazol-3 -amine;
5-(2-(3-((dibutylamino)methyl)phenyl)-1 H-pyrrolo[2,3 -b]pyridin-4 -yl)-1 H-indazol-3 -amine;
5-(2-(2 -fluoro-6-(piperidin-1 -ylmethyl)pyridin-4 -yl)-1 H-pyrrolo[2,3 -b]pyridin-4 -yl)-1 H-indazol-3 -amine;
5-(2-(2 -fluoro-6-(piperazin-1 -ylmethyl)pyridin-4 -yl)-1 H-pyrrolo[2,3 -b]pyridin-4 -yl)-1 H-indazol-3 -amine;
5-(2-(2 -fluoro-6-((4 -methylpiperazin-1 -yl)methyl)pyridin-4 -yl)-1 H-pyrrolo[2,3 -b]pyridin-4 -yl)-1 H-indazol-3 -amine;
tert-butyl 4-((4-(4-(3 -amino-1 H-indazol-5 -yl)-1 H-pyrrolo[2,3 -b]pyridin-2 -yl)-6 -fluoropyridin-2 -yl)methyl)piperazine-1 -carboxylate;
5-(2-(2-((tert-butylamino)methyl)-6 -fluoropyridin-4 -yl)-1 H-pyrrolo[2,3 -b]pyridin-4 -yl)-1 H-indazol-3 -amine;
5-(2-(2-((cyclohexylamino)methyl)-6 -fluoropyridin-4 -yl)-1 H-pyrrolo[2,3 -b]pyridin-4 -yl)-1 H-indazol-3 -amine;
5-(2-(2 -fluoro-6-((phenylamino)methyl)pyridin-4 -yl)-1 H-pyrrolo[2,3 -b]pyridin-4 -yl)-1 H-indazol-3 -amine;
4-(4-(3 -amino-1 H-indazol-5 -yl)-1 H-pyrrolo[2,3 -b]pyridin-2 -yl)-2,6 -difluorobenzamide
5-(2-(4-((dimethylamino)methyl)-3,5 -difluorophenyl)-1 H-pyrrolo[2,3 -b]pyridin-4 -yl)-1 H-indazol-3 -amine;
5-(2-(3,5 -difluoro-4-(piperidin-1 -ylmethyl)phenyl)-1 H-pyrrolo[2,3 -b]pyridin-4 -yl)-1 H-indazol-3 -amine;
5-(2-(3,5 -difluoro-4-((isopropyl(methyl)amino)methyl)phenyl)-1 H-pyrrolo[2,3 -b]pyridin-4 -yl)-1 H-indazol-3 -amine
5-(2-(4-((butyl(ethyl)amino)methyl)-3,5 -difluorophenyl)-1 H-pyrrolo[2,3 -b]pyridin-4 -yl)-1 H-indazol-3 -amine;
5-(2-(4-((dibutylamino)methyl)-3,5 -difluorophenyl)-1 H-pyrrolo[2,3 -b]pyridin-4 -yl)-1 H-indazol-3 -amine;
5-(2-(3,5 -difluoro-4-(3 -morpholinopropyl)phenyl)-1 H-pyrrolo[2,3 -b]pyridin-4 -yl)-1 H-indazol-3 -amine;
5-(2-(3,5 -difluoro-4-(3-(piperidin-1 -yl)propyl)phenyl)-1 H-pyrrolo[2,3 -b]pyridin-4 -yl)-1 H-indazol-3 -amine;
5-(2-(4-(3-(diethylamino)propyl)-3,5 -difluorophenyl)-1 H-pyrrolo[2,3 -b]pyridin-4 -yl)-1 H-indazol-3 -amine;
5-(2-(4-(3-(dibutylamino)propyl)-3,5 -difluorophenyl)-1 H-pyrrolo[2,3 -b]pyridin-4 -yl)-1 H-indazol-3 -amine;
7 -chloro-5-(2 -phenyl-1 H-pyrrolo[2,3 -b]pyridin-4 -yl)-1 H-indazol-3 -amine;
7 -chloro-5-(2-(3-(morpholinomethyl)phenyl)-1 H-pyrrolo[2,3 -b]pyridin-4 -yl)-1 H-indazol-3 -amine;
7 -chloro-5-(2-(4-(morpholinomethyl)phenyl)-1 H-pyrrolo[2,3 -b]pyridin-4 -yl)-1 H-indazol-3 -amine;
7 -chloro-5-(2-(2 -fluoro-6-(piperazin-1 -ylmethyl)pyridin-4 -yl)-1 H-pyrrolo[2,3 -b]pyridin-4 -yl)-1 H-indazol-3 -amine;
4-(3 -amino-7 -chloro-1 H-indazol-5 -yl)-N-(2-(piperidin-1 -yl)ethyl)-1 H-pyrrolo[2,3 -b]pyridine-2 -carboxamide;
7 -chloro-5-(2-(cyclohexylmethyl)-1 H-pyrrolo[2,3 -b]pyridin-4 -yl)-1 H-indazol-3 -amine;
7 -chloro-5-(2-(morpholinomethyl)-1 H-pyrrolo[2,3 -b]pyridin-4 -yl)-1 H-indazol-3 -amine;
7 -chloro-5-(2-(2 -morpholinoethyl)-1 H-pyrrolo[2,3 -b]pyridin-4 -yl)-1 H-indazol-3 -amine;
7 -phenyl-5-(2 -phenyl-1 H-pyrrolo[2,3 -b]pyridin-4 -yl)-1 H-indazol-3 -amine;
5-(2-(3-(morpholinomethyl)phenyl)-1 H-pyrrolo[2,3 -b]pyridin-4 -yl)-7 -phenyl-1 H-indazol-3 -amine;
5-(2-(4-(morpholinomethyl)phenyl)-1 H-pyrrolo[2,3 -b]pyridin-4 -yl)-7 -phenyl-1 H-indazol-3 -amine;
5-(2-(cyclohexylmethyl)-1 H-pyrrolo[2,3 -b]pyridin-4 -yl)-7-(3,3 -dimethylbut-1 -yn-1 -yl)-1 H-indazol-3 -amine; or
5-(2-(2-(Benzylthio)-6 -fluoropyridin-4 -yl)-1 H-pyrrolo[2,3 -b]pyridin-4 -yl)-1 H-indazol-3 -amine.

24. A pharmaceutical composition comprising a compound according in any anyone of claims 1 to 23, or a pharmaceutically acceptable salt or solvate thereof, and a pharmaceutically acceptable excipient.

25. A compound according to any one of claims 1 to 23, or a pharmaceutically acceptable salt or solvate thereof, or a pharmaceutical formulation according to claim 24:
(i) for use in therapy;
(ii) for use in the treatment of a disease or condition responsive to IKKalpha modulation;
(ii) for use in the treatment of a proliferative disorder (e.g. cancer); or
(iii) for use in the treatment of inflammation.
